(19) United States
(12) Patent Application Publication
(10) Pub. No.: US 2024/0246950 A1
(43) Pub. Date: Jul. 25, 2024

(54) Anilino-Pyrazole Derivatives, Compositions and Methods Thereof

(71) Applicant: Ensem Therapeutics, Waltham, MA (US)

(72) Inventor: Tao Liu, Waltham, MA (US)

(73) Assignee: Ensem Therapeutics, Waltham, MA (US)

(21) Appl. No.: 18/600,161

(22) Filed: Mar. 8, 2024

Related U.S. Application Data
(63) Continuation of application No. PCT/US2023/025368, filed on Jun. 15, 2023.
(60) Provisional application No. 63/407,247, filed on Sep. 16, 2022.
(60) Provisional application No. 63/352,872, filed on Jun. 16, 2022.

(51) Int. Cl. C07D 417/12; A61P 35/00; C07D 231/38; C07D 401/12; C07D 403/12; C07D 409/12; C07D 413/12; C07D 419/12; C07D 495/04

(52) U.S. Cl. CPC C07D 417/12; A61P 35/00; C07D 231/38; C07D 401/12; C07D 403/12; C07D 409/12; C07D 413/12; C07D 419/12; C07D 495/04

(57) ABSTRACT

The invention provides novel anilino-pyrazole derivatives as a cyclin-dependent kinase 2 (CDK2) inhibitors. The invention also provides pharmaceutical compositions comprising the compounds and methods thereof for treating various conditions, diseases and disorders associated with or related to CDK2 activities, or associated with abnormal cell growth, such as tumor growth and cancer.

### PRIORITY CLAIMS AND RELATED APPLICATIONS

This application claims the benefit of priority to U.S. Provisional Application Nos. 63/352,872, filed Jun. 16, 2022, and 63/407,247, filed Sep. 16, 2022, the entire content of each of which is incorporated herein by reference for all purposes.

### TECHNICAL FIELDS OF THE INVENTION

The invention generally relates to novel compounds and therapeutic uses thereof. More particularly, the invention provides novel anilino-pyrazole derivatives as a cyclin-dependent kinase 2 (CDK2) inhibitors. The invention also provides pharmaceutical compositions comprising the compounds and methods thereof for treating various conditions, diseases and disorders associated with or related to CDK2 activities, or associated with abnormal cell growth, such as tumor growth and cancer.

### BACKGROUND OF THE INVENTION

A broad range of aggressive cancers overexpress and/or amplify cyclin E gene (CCNE). Amplification of CCNE is associated with poor survival in cancer patients. (McDonald; et al. 2017 Cell 170(3):577-592; Etemadmoghadam, et al. 2010 PLoS One. 2010; 5(11): e15498.) CDK2, a.k.a. cell division protein kinase 2, is a member of the cyclin-dependent kinase family of serine/threonine protein kinases. CDK2 is an essential part of the abnormal growth processes of cancer cells. There is increasing evidence that CDK2 plays a critical role in tumorigenesis. Over expression of CDK2 causes abnormal regulation of cell-cycle, leading to hyperproliferation in cancer cells. Selective CDK2 inhibition thus may provide a therapeutic benefit against certain cancers. (Tadesse, et al. 2019 J Med. Chem. 62(9):4233-4251; Chohan, et al. 2015 Curr. Med. Chem. 22(2):237-63; Meijer, et al. 1999 Pharm. & Ther. 82 (2-3):279-284.)

Although several small-molecule CDK2 inhibitors have progressed to clinical trials, CDK2-selectivity is highly desired. Identifying selective CDK2inhibitors has been challenging due to the extreme similarity between the active sites of CDK2 and other CDKs, especially CDK1. Since CDK1 is an essential cyclin dependent kinase in the cell cycle, inhibition of CDK1 can lead to severe side effects. (Wood, et al. 2018 Cell Chem. Biol. 26(1): 121-130.e5; Brown, et al. 2015 Nature Comm. 6: 6769.)

There is an urgent need for potent and selective CDK2 inhibitors, in particular, compounds that are useful in treating diseases and conditions associated with overexpression of CCNE and CDK2 such as various types of cancer (e.g., gynecological cancer, breast cancer and gastric cancer).

### SUMMARY OF THE INVENTION

The invention provides novel CDK2 inhibitors that have been shown to exhibit favorable potency and selectivity profiles over known CDK2 inhibitors. The novel compounds selectively target, bind to and inhibit the activity of CDK2 and lead to cell cycle arrest, the induction of apoptosis, and the inhibition of tumor cell proliferation. The compounds are also orally available with pharmacokinetic profiles suitable for development into an orally administered therapeutic agent for treating cancer, e.g., breast cancer, ovarian cancer, gastric cancer or lung cancer.

In one aspect, the invention generally relates to a compound having the structural Formula I:

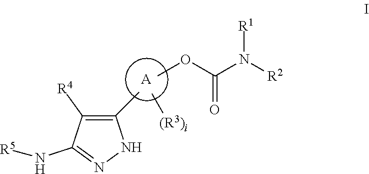

or a pharmaceutically acceptable form or an isotope derivative thereof,
wherein
Ring A is a 4- to 7-membered (e.g., 4-, 5-, 6- or 7-membered) carbocyclic ring;
each of R¹ and R² is independently selected from the group consisting of H, unsubstituted or substituted C₁-C₆ alkyl, and C₃-C₆ unsubstituted or substituted carbocyclic ring; or R¹ and R², together with the N atom they are bound to, are joined to form a 4- to 7-membered (e.g., 4-, 5-, 6- or 7-membered), unsubstituted or substituted heterocyclic ring;
each R³ is independently selected from the group consisting of halogen, OH, CN, unsubstituted or substituted C₁₋₆ alkyl, unsubstituted or substituted C₁₋₆ alkoxy, NO₂, NRR′, or two R³'s, along with the carbon atom(s) they are bound to, form a 3- to 5-membered (e.g., 3-, 4-, or 5-membered) unsubstituted or substituted carbocyclic ring;
R⁴ is H, halogen, C₁₋₄ alkyl, CN, NRR′, or C(O)NRR′;
R⁵ is

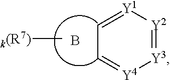

wherein
Ring B is a 4- to 7-membered (e.g., 4-, 5-, 6- or 7-membered) carbocyclic or heterocyclic ring optionally comprising a —S(O)₂—, —C(O)—, —S(O)₂NR—, —S(O)₂NRC(O)—, —C(O)NR—, —OC(O)NR—, or —C(O)NRC(O)— group;
each of Y¹ and Y² is CR⁶ or N, and each of Y³ and Y⁴ is C, CR⁶ or N; provided that not more than one of Y¹, Y², Y³ and Y⁴ is N, and either Y³ or Y⁴ is C and being the positions where R⁵ may be bonded to the rest of the compound;
each R⁶ is independently selected from the group consisting of halogen, OH, CN, unsubstituted or substituted C₁₋₆ alkyl, unsubstituted or substituted C₁₋₄ alkoxy, and C(O)NRR′;
each R⁷ is independently selected from the group consisting of halogen, OR, CN, unsubstituted or substituted C₁₋₆ alkyl, and unsubstituted or substituted C₁₋₄ alkoxy, or two R⁷'s, along with the carbon atom(s) they are bound to, form a 3- to 6-membered (e.g., 3-, 4-, 5- or 6-membered) unsubstituted or substituted carbocyclic or heterocyclic ring;
each of R and R′ is independently selected from H, unsubstituted or substituted C₁₋₆ alkyl, or unsubstituted or substituted 4- to 6-membered (e.g., 4-, 5- or 6-membered) carbocyclic ring, or where R and R′ are attached to the same N atom, together form an unsubstituted or substituted 4- to 6-membered (e.g., 4-, 5- or 6-membered) heterocyclic ring;
i is 0, 1, 2, 3, 4 or 5; and
k is 0, 1, 2, 3, 4 or 5.

In another aspect, the invention generally relates to a pharmaceutical composition comprising a compound disclosed herein and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing cancer, or a related disease or disorder, comprising administering to a subject in need thereof a therapeutically effective amount of a compound disclosed herein.

In yet another aspect, the invention generally relates to use of a compound disclosed herein, and a pharmaceutically acceptable excipient, carrier, or diluent, in preparation of a medicament for treating a disease or disorder.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

In this specification and the appended claims, the singular forms “a,” “an,” and “the” include plural reference, unless the context clearly dictates otherwise.

As used herein, the terms “administration” of or “administering” a disclosed compound encompasses the delivery to a subject of a compound as described herein, or a prodrug or other pharmaceutically acceptable form thereof, using any suitable formulation or route of administration, as discussed herein.

As used herein, the term “co-administer” refers to the presence of two pharmacological agents in a subject's body (e.g., in the blood) at the same time. The two pharmacological agents can be administered concurrently or sequentially.

The terms “disease”, “disorder” and “condition” are used interchangeably unless indicated otherwise.

As used herein, the terms “effective amount” or “therapeutically effective amount” refer to that amount of a compound or pharmaceutical composition described herein that is sufficient to affect the intended application including, but not limited to, disease treatment, as illustrated below.

In some embodiments, the amount is that effective for stop the progression or effect reduction of an inflammatory disease or disorder. In some embodiments, the amount is that effective for stop the progression or effect reduction of an immune system disorders. In some embodiments, the amount is that effective to stop the progression or effect reduction of an autoimmune disease or disorder. In some embodiments, the amount is that effective for stop the progression or effect reduction of a cardiovascular disease or disorder. In some embodiments, the amount is that effective for detectable killing or inhibition of the growth or spread of cancer cells; the size or number of tumors; or other measure of the level, stage, progression or severity of the cancer. In some embodiments, the amount is that effective for stop the progression or effect reduction of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, or neurodegenerative diseases. In some embodiments, the amount is that effective for stop the progression or effect reduction of Parkinson's disease (PD). In some embodiments, the amount is that effective for stop the progression or effect reduction of Alzheimer's disease (AD).

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. Such amount may be administered as a single dosage or according to a regimen. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “unsubstituted or substituted” and “optionally substituted” are used interchangeably and refer to where a given chemical moiety (e.g., an alkyl group) can (but is not required to) be bonded to other substituents (e.g., heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, CN, —COOH, —CH₂CN, —O—C₁-C₆ alkyl, C₁-C₆ alkyl, —OC₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —NHC(O)C₁-C₆ alkyl, —C(O)NHC₁-C₆ alkyl, —S(O)₂—C₁-C₆ alkyl, —S(O)NHC₁-C₆ alkyl, and S(O)N(C₁-C₆ alkyl)₂.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

As used herein, the term an “isolated” or “substantially isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “purified” when used in reference to a molecule, it means that the concentration of the molecule being purified has been increased relative to molecules associated with it in its natural environment, or environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

Definitions of specific functional groups and chemical terms are described in more detail below. When a range of values is listed, it is intended to encompass each value and sub-range within the range. By way of example, “C₁₋₆ alkyl” is intended to encompass, C₁, C₂, C₃, C₄, C₅, C₆, C₁₋₄, C₁₋₃, C₁₋₂, C₂₋₅, C₂₋₄, C₃₋₆, C₃₋₅ and C₄₋₆ alkyl groups.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ (e.g., C₁, C₂, C₃, C₄, C₅ or C₆) alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{X})₃, —OR^{X}, —SR^{X}, —OC(O)—R^{X}, —N(R^{X})₂, —C(O)R^{X}, —C(O)OR^{X}, —OC(O)N(R^{X})₂, —C(O)N(R^{X})₂, —N(R^{X})C(O)OR^{X}, —N(R^{X})C(O)R^{X}, —N(R^{X})C(O)N(R^{X})₂, —N(R^{X})C(NR^{X})N(R^{X})₂, —N(R^{X})S(O)ₜN(R^{X})₂ (where t is 1 or 2), —P(═O)(R^{X})(R^{X}), or —O—P(═O)(OR^{X})₂ wherein each R^{X} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

Unless otherwise specifically defined, the term “aromatic” or “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, H, halogen, —O—C₁-C₆ alkyl, C₁-C₆ alkyl, —C₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆alkyl, NH₂, NH(C₁-C₆ alkyl), N(C₁-C₆ alkyl)₂, —S(O)₂—C₁-C₆ alkyl, —S(O)NHC₁-C₆alkyl, and S(O)N(C₁-C₆ alkyl)₂. The substituents can themselves be optionally substituted. Furthermore, when containing two fused rings the aryl groups herein defined may have an unsaturated or partially saturated ring fused with a fully unsaturated ring. Exemplary ring systems of these aryl groups include indanyl, indenyl, tetrahydronaphthalenyl, and tetrahydrobenzoannulenyl.

The term “halogen” or “halo” refers to fluorine (F), chlorine (Cl), bromine (Br) and iodine (I).

As used herein, the terms “heteroaryl” or “hetero-aromatic” refer to groups having 5 to 14 ring atoms, preferably 5, 6, 9, or 10 ring atoms; having 6, 10, or 14 p electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

Heteroaryl groups may be substituted with 0, 1, 2, 3, or 4 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂, and (NZ₁Z₂)carbonyl. The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, and formyl. Representative examples of NZ₁Z₂ include, but are not limited to, amino, methylamino, acetylamino, and acetylmethylamino.

As used herein, the term “alkoxy” refers to an —O-alkyl radical.

As used herein, the terms “cycloalkyl” and “carbocyclyl” each refers to a monocyclic or polycyclic radical that contains only carbon and hydrogen, and can be saturated or partially unsaturated. Unless stated otherwise in the specification, the term is intended to include both substituted and unsubstituted cycloalkyl groups. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₅), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O),N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “heterocycloalkyl” refers to a cycloalkyl radical, which have one or more skeletal chain atoms selected from an atom other than carbon, e.g., O, N, S, P or combinations thereof. Unless stated otherwise in the specification, the term is intended to include both substituted and unsubstituted heterocycloalkyl groups. Illustrative examples of heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, 1-cyclopropyl-4-methyl-piperazin-2-yl. 2-pyrrolinyl, 3-pyrrolinyl, dihydro-2H-pyranyl, 1,2,3,4-tetrahydropyridine, 3,4-dihydro-2H-[1,4]oxazine, etc.

As used herein, the terms “heterocycle”, “heterocyclic” or “heterocyclo” refer to fully saturated or partially unsaturated cyclic groups, for example, 3- to 7-membered monocyclic, 7 to 12 membered bicyclic, or 10 to 15 membered spirocyclic or tricyclic ring systems, which have at least one heteroatom (selected from the group consisting of N, O, and S) in at least one ring, wherein 0, 1, 2 or 3 atoms of each ring may be substituted by a substituent. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. A heterocyclic group is optionally substituted. Examples of heterocyclic groups include, but not limited to, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1, 1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide.

### DETAILED DESCRIPTION OF THE INVENTION

The invention is based in part on the discovery of novel CDK2 inhibitors that selectively target, bind to and inhibit the activity of CDK2. The novel compound have been shown to exhibit favorable potency and selectivity profiles over known CDK2 inhibitors, such as PF-07104091. The compounds exhibit superior DMPK profiles suitable for development into an orally administered therapeutic agent for treating cancer, e.g., breast cancer, ovarian cancer, gastric cancer or lung cancer.

In one aspect, the invention generally relates to a compound having the structural Formula I:

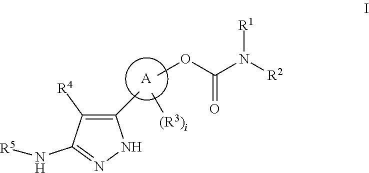

or a pharmaceutically acceptable form or an isotope derivative thereof,
wherein
Ring A is a 4- to 7-membered (e.g., 4-, 5-, 6- or 7-membered) carbocyclic ring;
each of R¹ and R² is independently selected from the group consisting of H, unsubstituted or substituted C₁-C₆ alkyl, and C₃-C₆ unsubstituted or substituted carbocyclic ring; or R¹ and R², together with the N atom they are bound to, are joined to form a 4- to 7-membered (e.g., 4-, 5-, 6- or 7-membered), unsubstituted or substituted heterocyclic ring;
each R³ is independently selected from the group consisting of halogen, OH, CN, unsubstituted or substituted C₁₋₆ alkyl, unsubstituted or substituted C₁₋₆ alkoxy, NO₂, NRR′, or two R³'s, along with the carbon atom(s) they are bound to, form a 3- to 5-membered (e.g., 3-, 4-, or 5-membered) unsubstituted or substituted carbocyclic ring;
R⁴ is H, halogen, C₁₋₄ alkyl, CN, NRR′, or C(O)NRR′;
R⁵ is

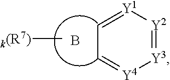

wherein
Ring B is a 4- to 7-membered (e.g., 4-, 5-, 6- or 7-membered) carbocyclic or heterocyclic ring optionally comprising a —S(O)₂—, —C(O)—, —S(O)₂NR—, —S(O)₂NRC(O)—, —C(O)NR—, —OC(O)NR—, or —C(O)NRC(O)— group;
each of Y¹ and Y² is CR⁶ or N, and each of Y³ and Y⁴ is C, CR⁶ or N; provided that not more than one of Y¹, Y², Y³ and Y⁴ is N, and either Y³ or Y⁴ is C and being the positions where R⁵ may be bonded to the rest of the compound;
each R⁶ is independently selected from the group consisting of halogen, OH, CN, unsubstituted or substituted C₁₋₆ alkyl, unsubstituted or substituted C₁₋₄ alkoxy, and C(O)NRR′;
each R⁷ is independently selected from the group consisting of halogen, OR, CN, unsubstituted or substituted C₁₋₆ alkyl, and unsubstituted or substituted C₁₋₄ alkoxy, or two R⁷'s, along with the carbon atom(s) they are bound to, form a 3- to 6-membered (e.g., 3-, 4-, 5- or 6-membered) unsubstituted or substituted carbocyclic or heterocyclic ring;
each of R and R′ is independently selected from H, unsubstituted or substituted C₁₋₆ alkyl, or unsubstituted or substituted 4- to 6-membered (e.g., 4-, 5- or 6-membered) carbocyclic ring, or where R and R′ are attached to the same N atom, together form an unsubstituted or substituted 4- to 6-membered (e.g., 4-, 5- or 6-membered) heterocyclic ring;
i is 0, 1, 2, 3, 4 or 5; and
k is 0, 1, 2, 3, 4 or 5.

In certain embodiments of (I), none of Y¹, Y², Y³ and Y⁴ is N and R⁵ is

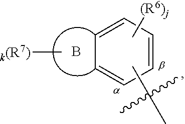

wherein
each R⁶ is independently selected from the group consisting of halogen, OH, CN, unsubstituted or substituted C₁₋₆ alkyl, unsubstituted or substituted C₁₋₄ alkoxy, and C(O)NRR′;
α and β refer to positions where R⁵ may be bonded to the rest of the compound; and j is 0, 1, 2 or 3.

In certain embodiments, R⁵ is connected to the rest of the compound at the β position:

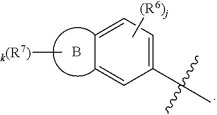

In certain embodiments, R⁵ is connected to the rest of the compound at the α position:

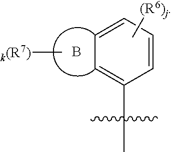

In certain embodiments of (I), at least one of Y¹, Y², Y³ and Y⁴ is N.

In certain embodiments of (I), only one of Y¹, Y², Y³ and Y⁴ is N.

In certain embodiments of (I), Y³ is C and R⁵ is connected to the rest of the compound at the Y³ position:

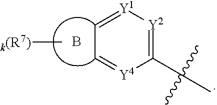

In certain embodiments, R⁵ is selected from:

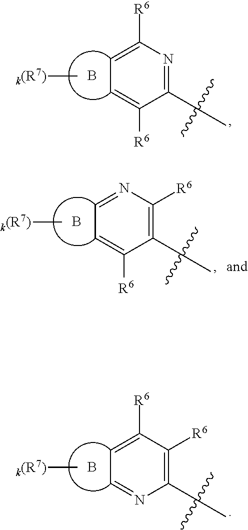

In certain embodiments of (I), Y⁴ is C and R⁵ is connected to the rest of the compound at the Y⁴ position:

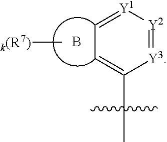

In certain embodiments, R⁵ is selected from:

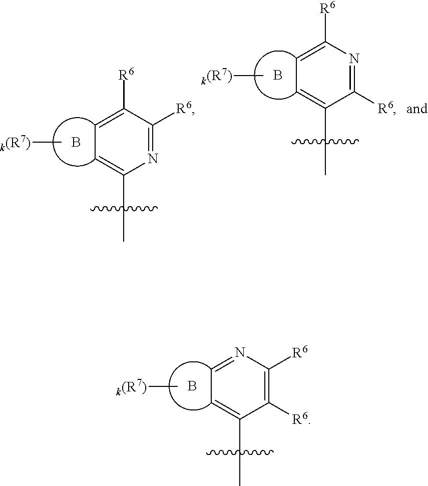

Ring B may be selected from:

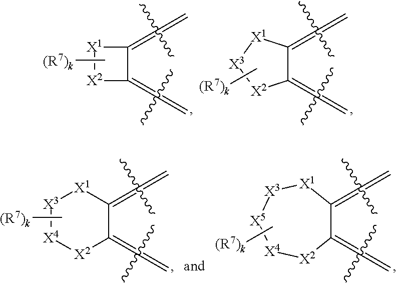

wherein
each of X¹, X², X³, X⁴ and X⁵ is independently selected from S(O)₂, C(O), NH, CH₂ and O, provided that
two neighboring X¹, X², X³, X⁴ or X⁵ are not each selected from S(O)₂ and C(O); and
two neighboring X¹, X², X³, X⁴ or X⁵ are not each selected from NH and O.

In certain embodiments, one or both of X₁ and X₂ is selected from S(O)₂ and C(O); and none of X₃, X₄ and X₅ is S(O)₂ or C(O).

In certain embodiments, neither of X₁ and X₂ is S(O)₂ or C(O); and one of X₃, X₄ and X₅ is S(O)₂ or C(O).

In certain embodiments, none of X₁, X₂, X₃, X₄ and X₅ is S(O)₂ or C(O); and at least one of X₁, X₂, X₃, X₄ and X₅ is CH₂, wherein
one or both H's in the CH₂ are substituted with F;
one of H's is substituted with CN and the other H is unsubstituted; or
one of H's is substituted with CN and the other H is substituted with OR.

In certain embodiments, R is selected from:

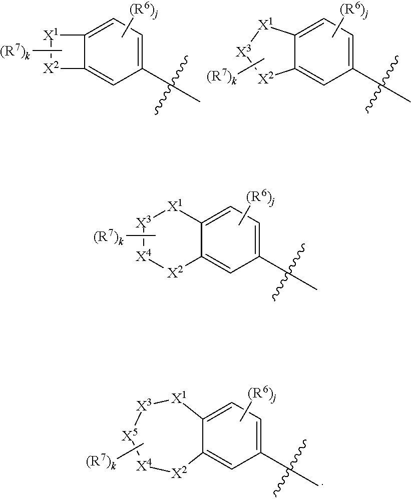

In certain embodiments, R⁵ is selected from:

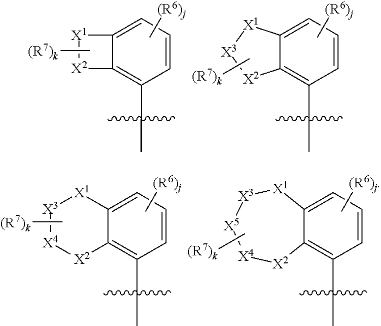

In certain embodiments, R⁵ is selected from:

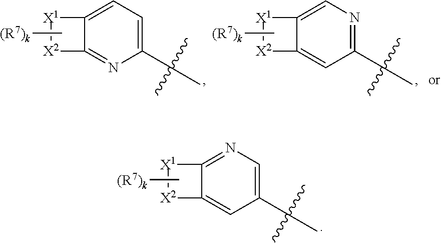

In certain embodiments, R⁵ is selected from:

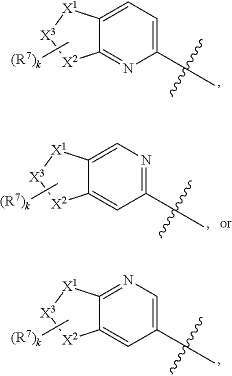

In certain embodiments, R⁵ is selected from:

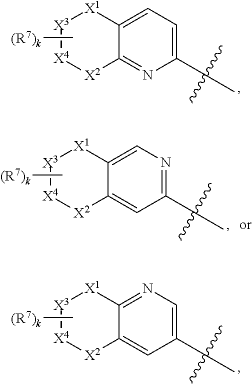

In certain embodiments, R⁵ is selected from:

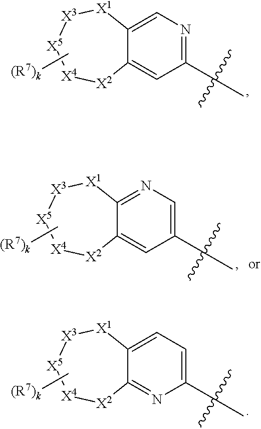

In certain embodiments, R⁵ is selected from:

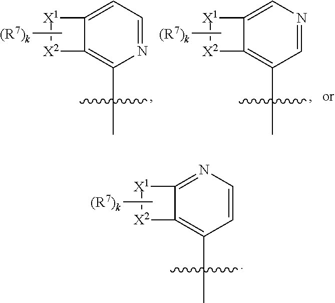

In certain embodiments, R⁵ is selected from:

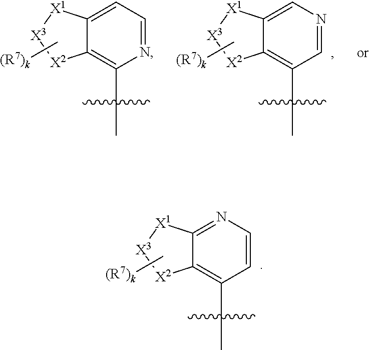

In certain embodiments, R⁵ is selected from:

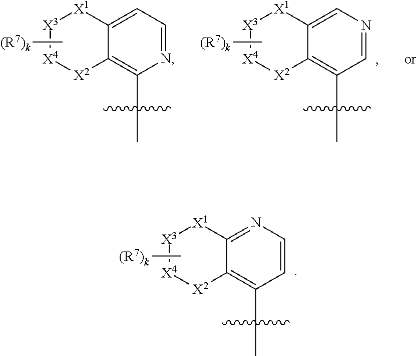

In certain embodiments, R⁵ is selected from:

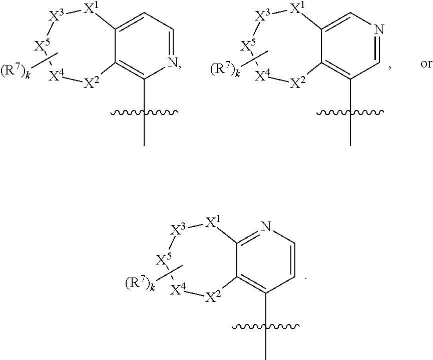

In certain embodiments, two R⁷'s, along with the carbon atom they are bound to, form a 3- to 6-membered (e.g., 3-, 4-, 5- or 6-membered) unsubstituted or substituted carbocyclic ring.

In certain embodiments, two R⁷'s, along with the carbon atom they are bound to, form a 3- to 6-membered (e.g., 3-, 4-, 5- or 6-membered) unsubstituted or substituted heterocyclic ring comprising one or more ring heteroatoms selected from O, S and N.

In certain embodiments, R⁵ is selected from:

TABLE 1

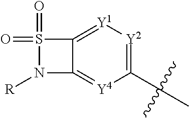

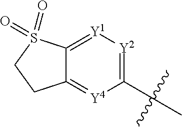

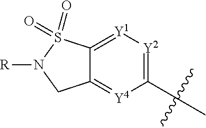

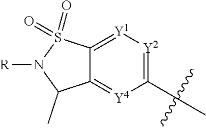

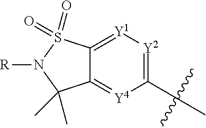

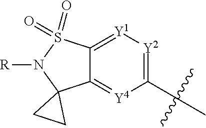

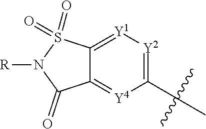

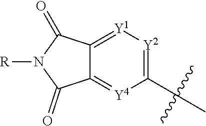

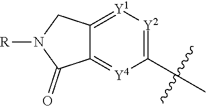

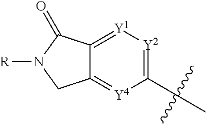

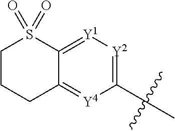

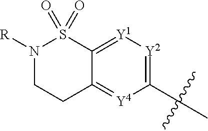

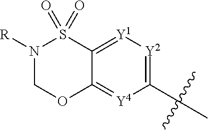

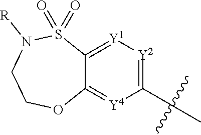

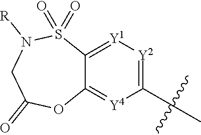

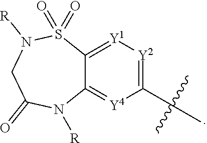

In certain embodiments, R⁵ is selected from:

TABLE 2

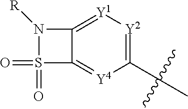

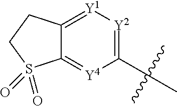

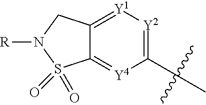

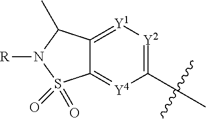

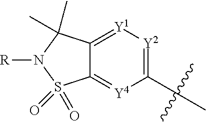

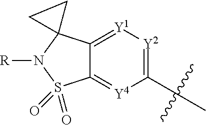

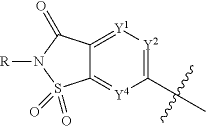

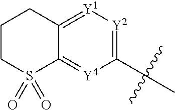

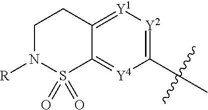

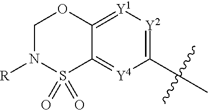

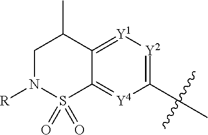

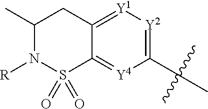

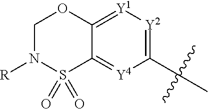

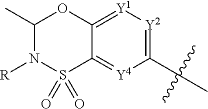

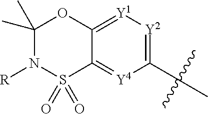

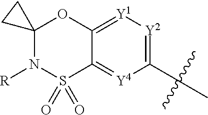

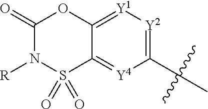

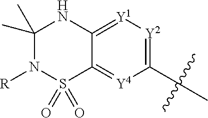

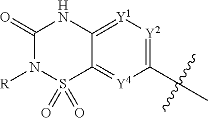

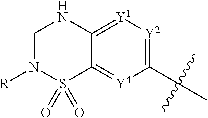

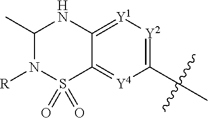

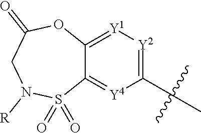

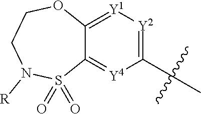

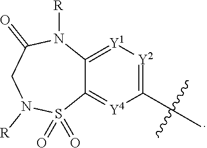

In certain embodiments, R⁵ is selected from:

TABLE 3

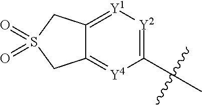

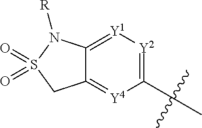

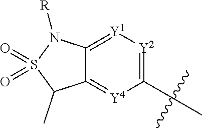

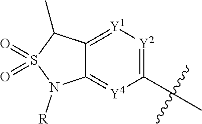

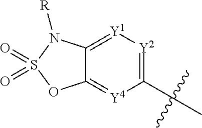

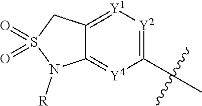

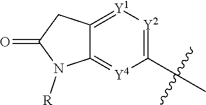

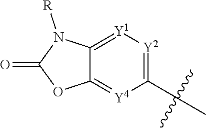

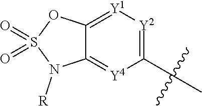

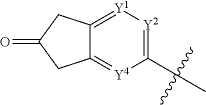

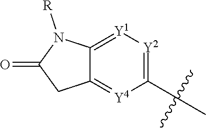

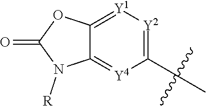

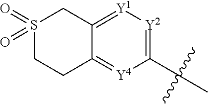

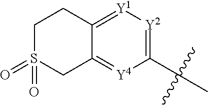

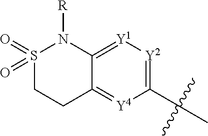

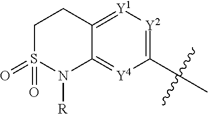

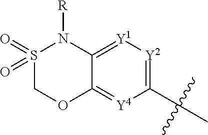

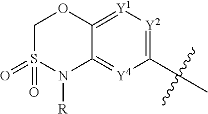

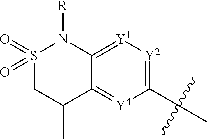

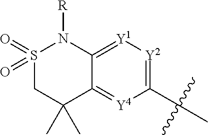

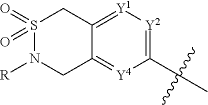

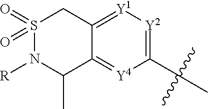

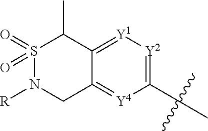

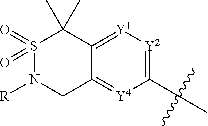

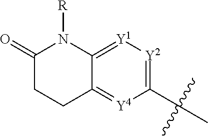

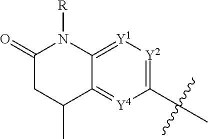

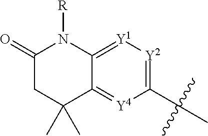

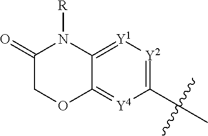

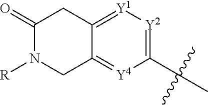

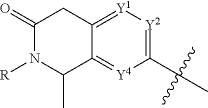

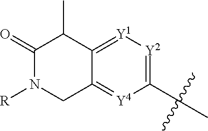

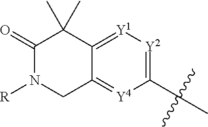

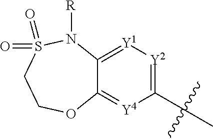

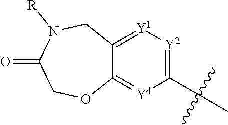

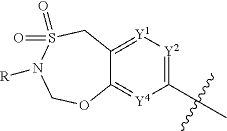

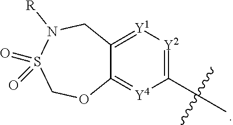

In certain embodiments of Tables 1-3, Y¹ is N, Y² is CH and Y⁴ is CH.

In certain embodiments of Tables 1-3, Y¹ is CH, Y² is N and Y⁴ is CH.

In certain embodiments of Tables 1-3, Y¹ is CH, Y² is CH and Y⁴ is N.

In certain embodiments of Tables 1-3, each of Y¹, Y² and Y⁴ is CH.

In certain embodiments, R⁵ is selected from:

TABLE 4

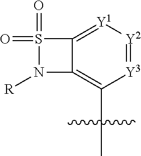

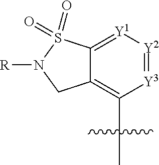

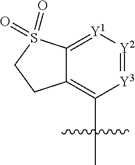

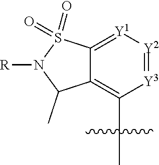

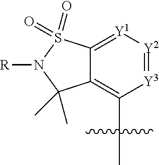

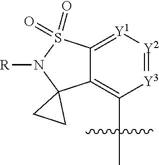

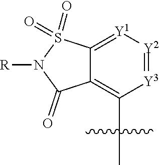

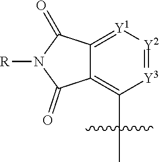

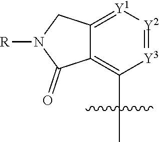

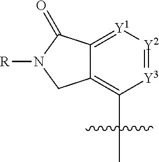

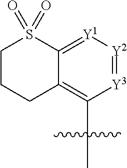

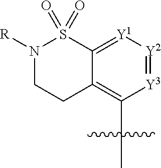

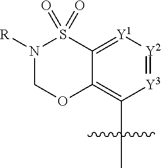

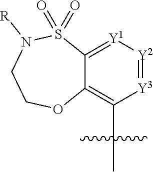

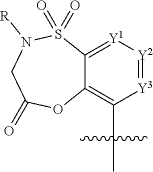

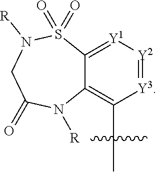

In certain embodiments, R⁵ is selected from:

TABLE 5

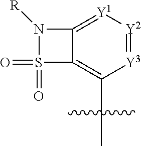

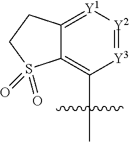

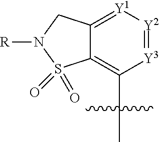

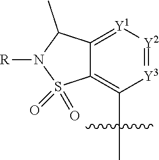

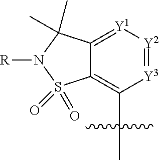

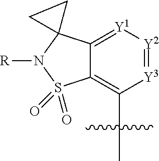

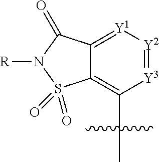

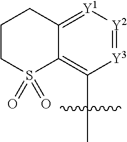

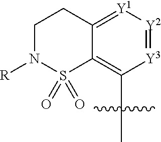

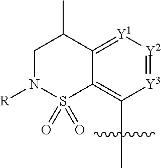

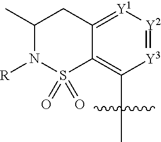

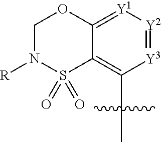

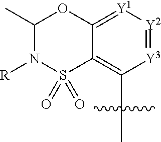

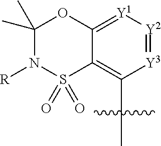

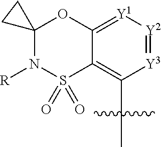

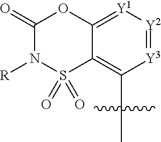

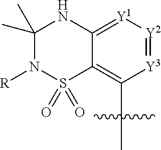

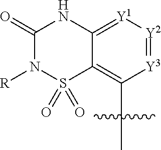

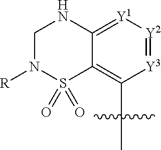

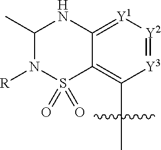

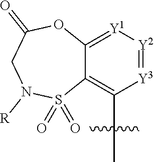

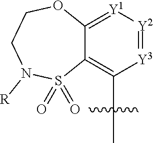

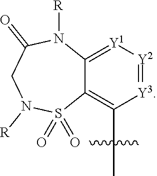

In certain embodiments, R⁵ is selected from:

TABLE 6

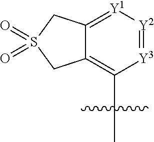

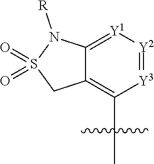

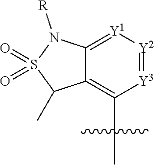

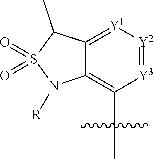

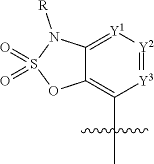

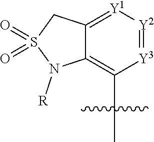

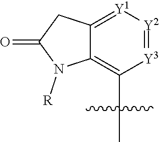

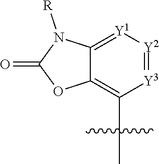

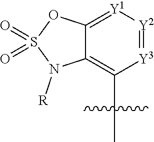

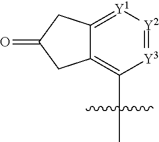

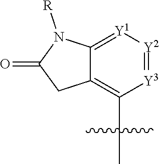

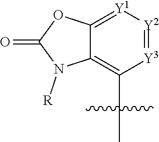

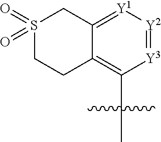

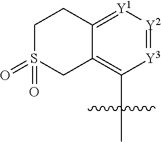

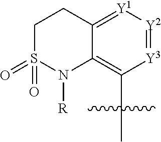

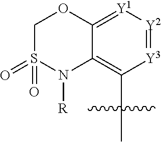

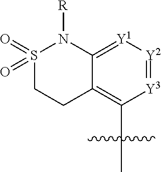

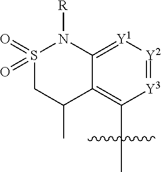

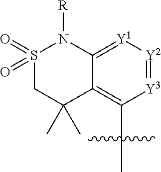

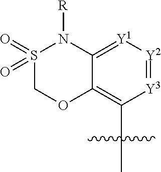

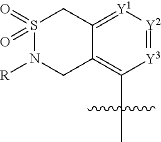

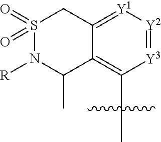

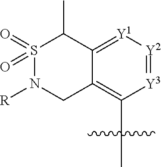

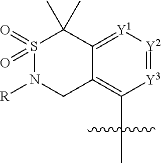

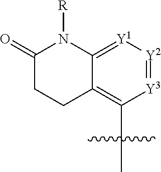

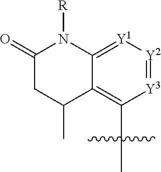

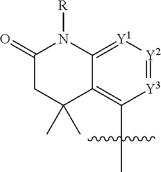

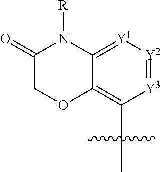

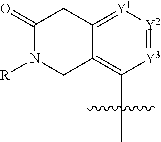

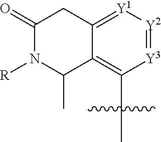

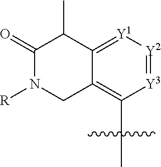

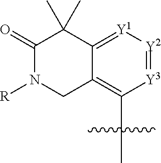

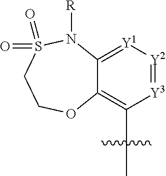

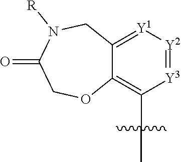

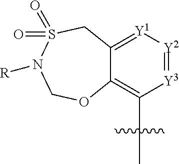

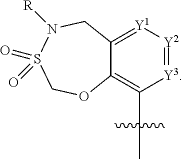

In certain embodiments of Tables 4-6, Y¹ is N, Y² is CH and Y³ is CH.

In certain embodiments of Tables 4-6, Y¹ is CH, Y² is N and Y³ is CH.

In certain embodiments of Tables 4-6, Y¹ is CH, Y² is CH and Y³ is N.

In certain embodiments of Tables 4-6, each of Y¹, Y² and Y³ is CH.

In certain embodiments of R⁵, R is H.

In certain embodiments of R⁵, R is an unsubstituted or substituted C₁₋₆ alkyl.

In certain embodiments, R is methyl.

In certain embodiments, R¹ is H and R² is an unsubstituted or substituted linear or branched C₁-C₆ alkyl.

In certain embodiments, each of R¹ and R² is independently an unsubstituted or substituted linear or branched C₁-C₆ alkyl.

In certain embodiments, R¹ and R², together with the N atom they are bound to, are joined to form a 4- to 7-membered (e.g., 4-, 5-, 6- or 7-membered), unsubstituted or substituted heterocyclic ring.

In certain embodiments, Ring A is a 4- to 7-membered (e.g., 4-, 5-, 6- or 7-membered) carbocyclic ring.

In certain embodiments, Ring A is selected from:

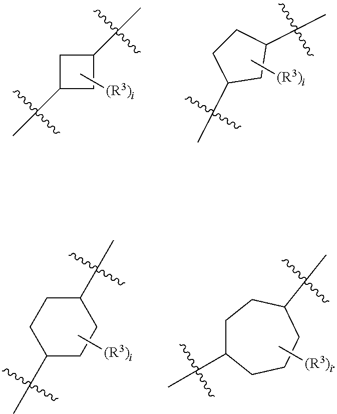

In certain embodiments, Ring A is selected from:

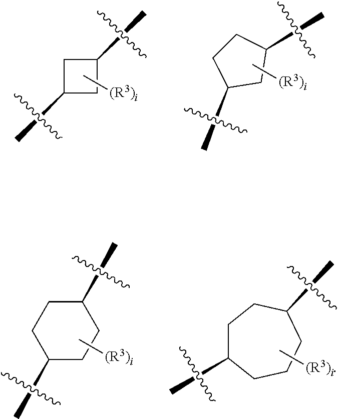

In certain embodiments, Ring A is a 5-membered carbocyclic ring.

In certain embodiments, Ring A is:

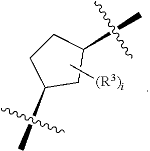

In certain embodiments, i is 0.

In certain embodiments, compounds of the invention have the structural formula:

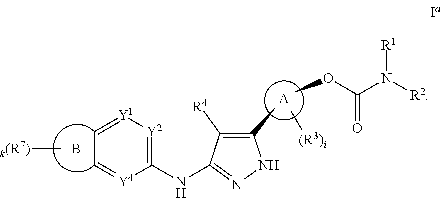

In certain embodiments of (I^{a}), Y¹ is N, Y² is CH and Y⁴ is CH.

In certain embodiments of (I^{a}), Y¹ is CH, Y² is N and Y⁴ is CH.

In certain embodiments of (I^{a}), Y¹ is CH, Y² is CH and Y⁴ is N.

In certain embodiments, compounds of the invention have the structural formula:

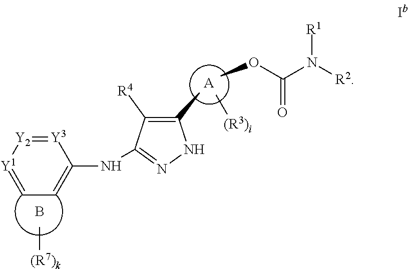

In certain embodiments of (I^{b}), Y¹ is N, Y² is CH and Y³ is CH.

In certain embodiments of (I^{b}), Y¹ is CH, Y² is N and Y³ is CH.

In certain embodiments of (I^{b}), Y¹ is CH, Y² is CH and Y³ is N.

In certain embodiments of (I^{a}) and (I^{b}), Ring B is 5-membered carbocyclic ring.

In certain embodiments of (I^{a}) and (I^{b}), Ring B is 5-membered heterocyclic ring.

In certain embodiments of (I^{a}) and (I^{b}), Ring B is 6-membered carbocyclic ring.

In certain embodiments of (I^{a}) and (I^{b}), Ring B is 6-membered heterocyclic ring.

In certain embodiments of (I^{a}) and (I^{b}), Ring B comprises a —S(O)₂NH— group.

In certain embodiments of (I^{a}) and (I^{b}), Ring B comprises a —S(O)₂— group.

In certain embodiments of (I^{a}) and (I^{b}), Ring B comprises a —C(O)NH— group.

In certain embodiments of (I^{a}) and (I^{b}), Ring B comprises a CH₂ group.

In certain embodiments of (I^{a}) and (Ie), Ring B comprises a C(CN)R″ group, wherein R″ is H, OH, C₁₋₄ alkyl or O—C₁₋₄ alkyl.

In certain embodiments of (I^{a}) and (I^{b}), j is 0.

In certain embodiments of (I^{a}) and (Ie), j is 1.

In certain embodiments of (I^{a}) and (I^{b}), k is 0.

In certain embodiments of (I^{a}) and (I¹), k is 1.

In certain embodiments of (I^{a}) and (I¹), k is 2.

In certain embodiments of (I^{a}) and (I′), R⁷ is an unsubstituted or substituted C₁₋₆ alkyl.

Exemplary compounds of the invention include:

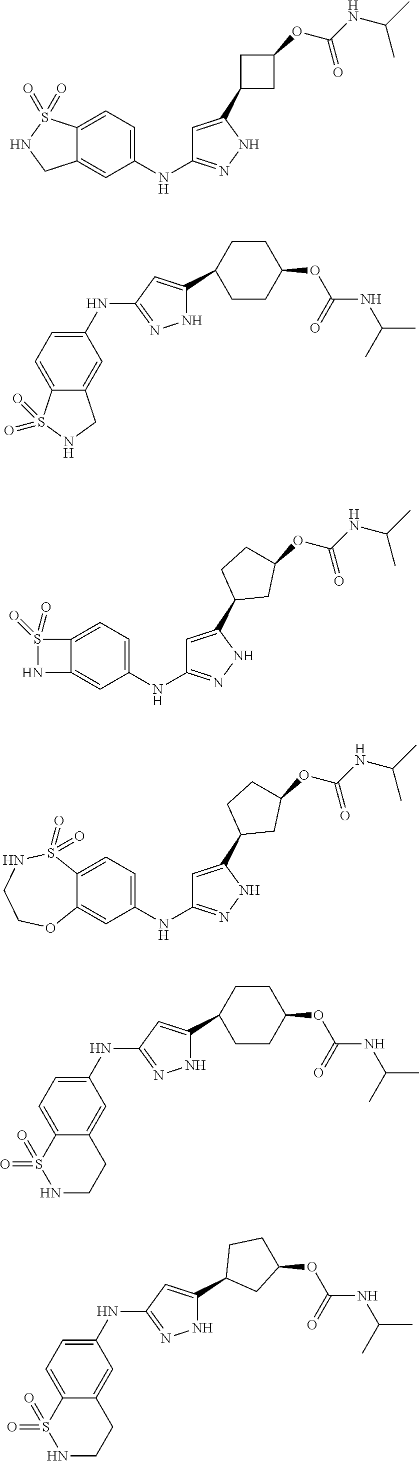 [SMILES: CC(C)NC(=O)O[C@H]1C[C@@H](c2cc(Nc3ccc4c(c3)CNS4(=O)=O)n[nH]2)C1 ; CC(C)NC(=O)O[C@H]1CC[C@@H](c2cc(Nc3ccc4c(c3)CNS4(=O)=O)n[nH]2)CC1 ; CC(C)NC(=O)O[C@@H]1CC[C@H](c2cc(Nc3ccc4c(c3)NS4(=O)=O)n[nH]2)C1 ; CC(C)NC(=O)O[C@@H]1CC[C@H](c2cc(Nc3ccc4c(c3)OCCNS4(=O)=O)n[nH]2)C1 ; CC(C)NC(=O)O[C@H]1CC[C@@H](c2cc(Nc3ccc4c(c3)CCNS4(=O)=O)n[nH]2)CC1 ; CC(C)NC(=O)O[C@@H]1CC[C@H](c2cc(Nc3ccc4c(c3)CCNS4(=O)=O)n[nH]2)C1]

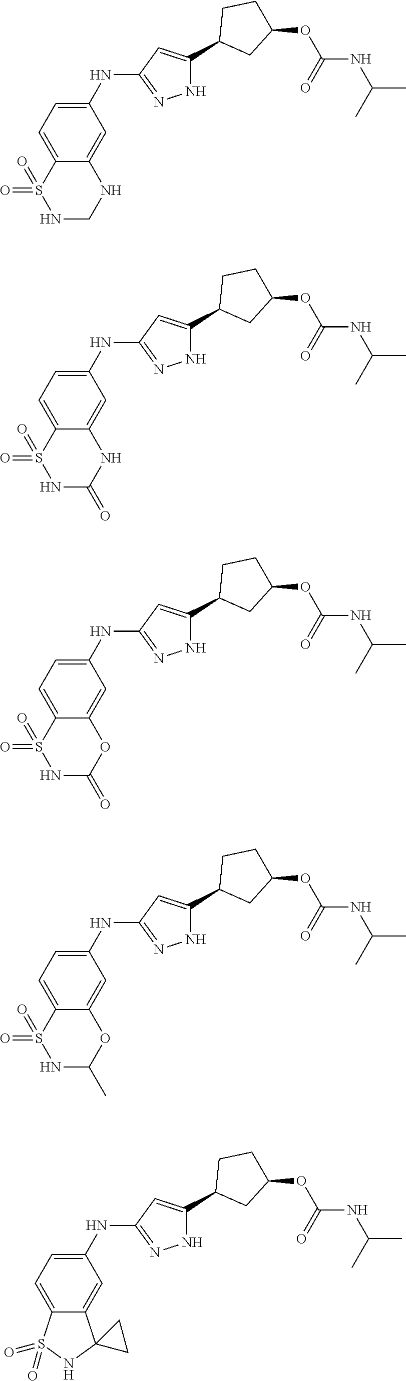 [SMILES: CC(C)NC(=O)O[C@@H]1CC[C@H](c2cc(Nc3ccc4c(c3)NCNS4(=O)=O)n[nH]2)C1 ; CC(C)NC(=O)O[C@@H]1CC[C@H](c2cc(Nc3ccc4c(c3)NC(=O)NS4(=O)=O)n[nH]2)C1 ; CC(C)NC(=O)O[C@@H]1CC[C@H](c2cc(Nc3ccc4c(c3)OC(=O)NS4(=O)=O)n[nH]2)C1 ; CC(C)NC(=O)O[C@@H]1CC[C@H](c2cc(Nc3ccc4c(c3)OC(C)NS4(=O)=O)n[nH]2)C1 ; CC(C)NC(=O)O[C@@H]1CC[C@H](c2cc(Nc3ccc4c(c3)C3(CC3)NS4(=O)=O)n[nH]2)C1]

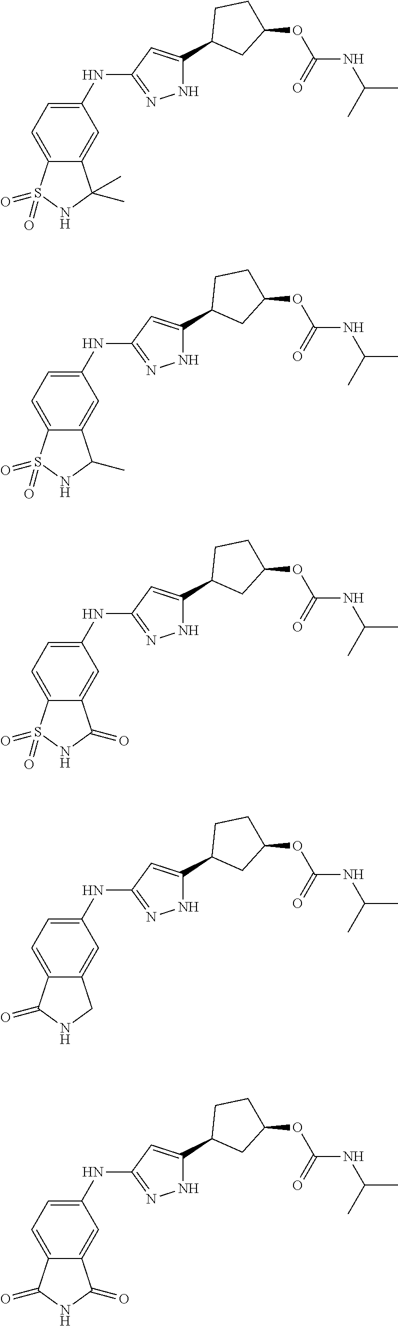 [SMILES: CC(C)NC(=O)O[C@@H]1CC[C@H](c2cc(Nc3ccc4c(c3)C(C)(C)NS4(=O)=O)n[nH]2)C1 ; CC(C)NC(=O)O[C@@H]1CC[C@H](c2cc(Nc3ccc4c(c3)C(C)NS4(=O)=O)n[nH]2)C1 ; CC(C)NC(=O)O[C@@H]1CC[C@H](c2cc(Nc3ccc4c(c3)C(=O)NS4(=O)=O)n[nH]2)C1 ; CC(C)NC(=O)O[C@@H]1CC[C@H](c2cc(Nc3ccc4c(c3)CNC4=O)n[nH]2)C1 ; CC(C)NC(=O)O[C@@H]1CC[C@H](c2cc(Nc3ccc4c(c3)C(=O)NC4=O)n[nH]2)C1]

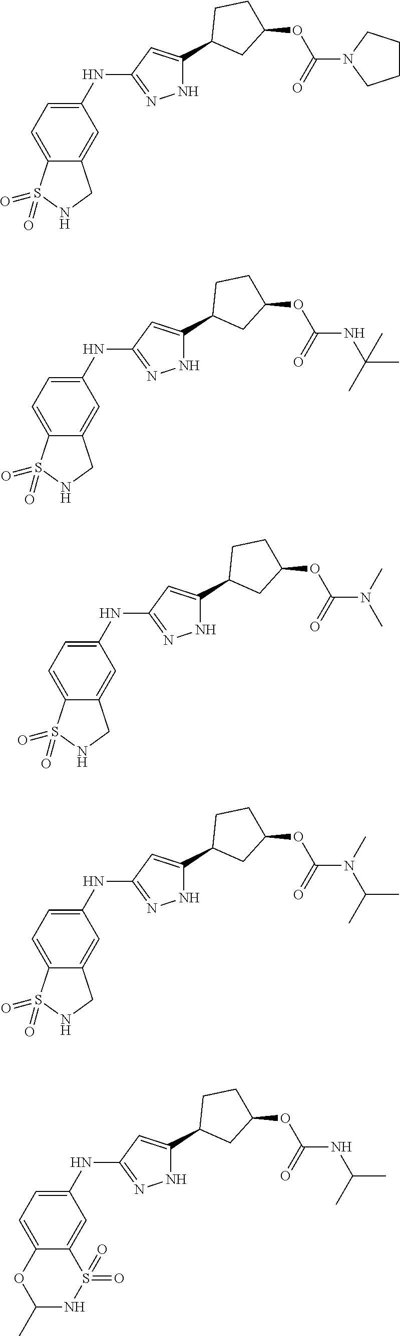 [SMILES: O=C(O[C@@H]1CC[C@H](c2cc(Nc3ccc4c(c3)CNS4(=O)=O)n[nH]2)C1)N1CCCC1 ; CC(C)(C)NC(=O)O[C@@H]1CC[C@H](c2cc(Nc3ccc4c(c3)CNS4(=O)=O)n[nH]2)C1 ; CN(C)C(=O)O[C@@H]1CC[C@H](c2cc(Nc3ccc4c(c3)CNS4(=O)=O)n[nH]2)C1 ; CC(C)N(C)C(=O)O[C@@H]1CC[C@H](c2cc(Nc3ccc4c(c3)CNS4(=O)=O)n[nH]2)C1 ; CC(C)NC(=O)O[C@@H]1CC[C@H](c2cc(Nc3ccc4c(c3)S(=O)(=O)NC(C)O4)n[nH]2)C1]

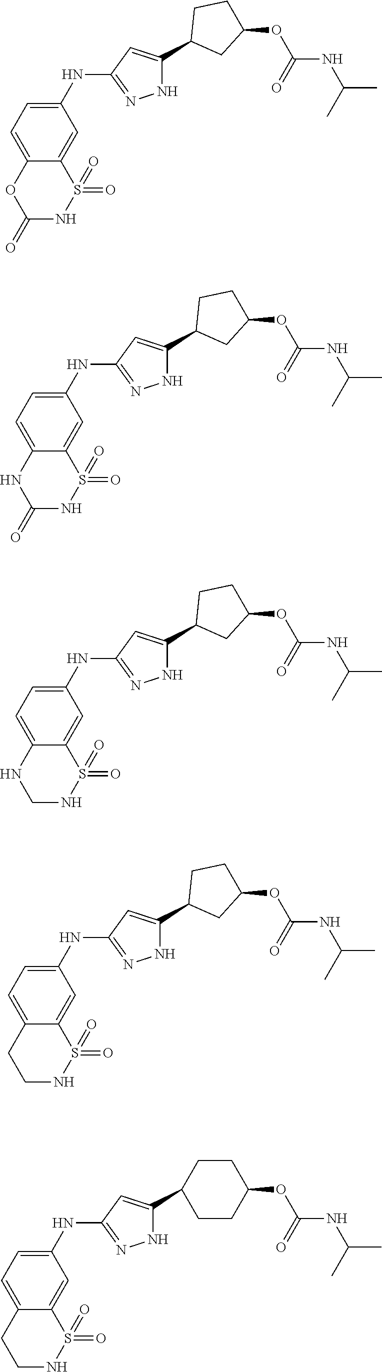 [SMILES: CC(C)NC(=O)O[C@@H]1CC[C@H](c2cc(Nc3ccc4c(c3)S(=O)(=O)NC(=O)O4)n[nH]2)C1 ; CC(C)NC(=O)O[C@@H]1CC[C@H](c2cc(Nc3ccc4c(c3)S(=O)(=O)NC(=O)N4)n[nH]2)C1 ; CC(C)NC(=O)O[C@@H]1CC[C@H](c2cc(Nc3ccc4c(c3)S(=O)(=O)NCN4)n[nH]2)C1 ; CC(C)NC(=O)O[C@@H]1CC[C@H](c2cc(Nc3ccc4c(c3)S(=O)(=O)NCC4)n[nH]2)C1 ; CC(C)NC(=O)O[C@H]1CC[C@@H](c2cc(Nc3ccc4c(c3)S(=O)(=O)NCC4)n[nH]2)CC1]

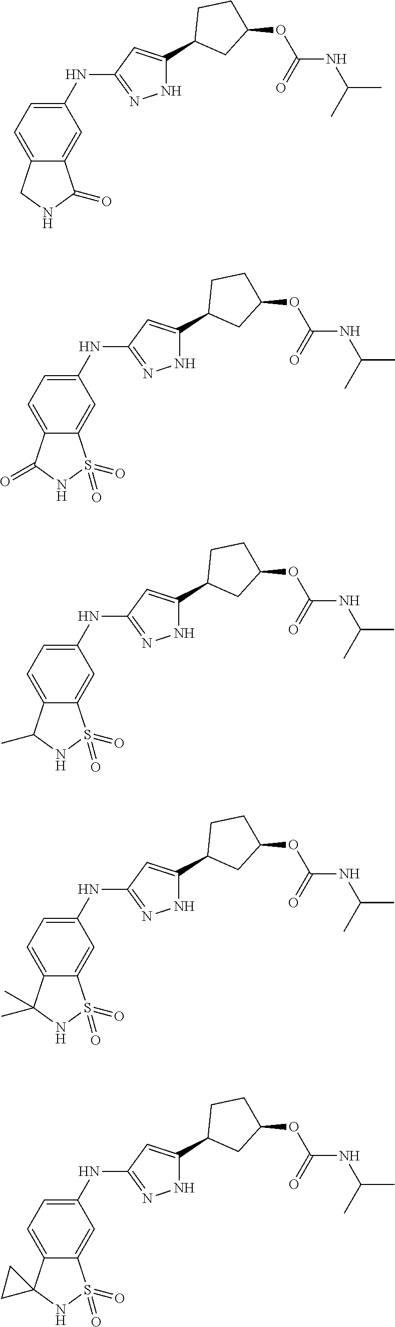

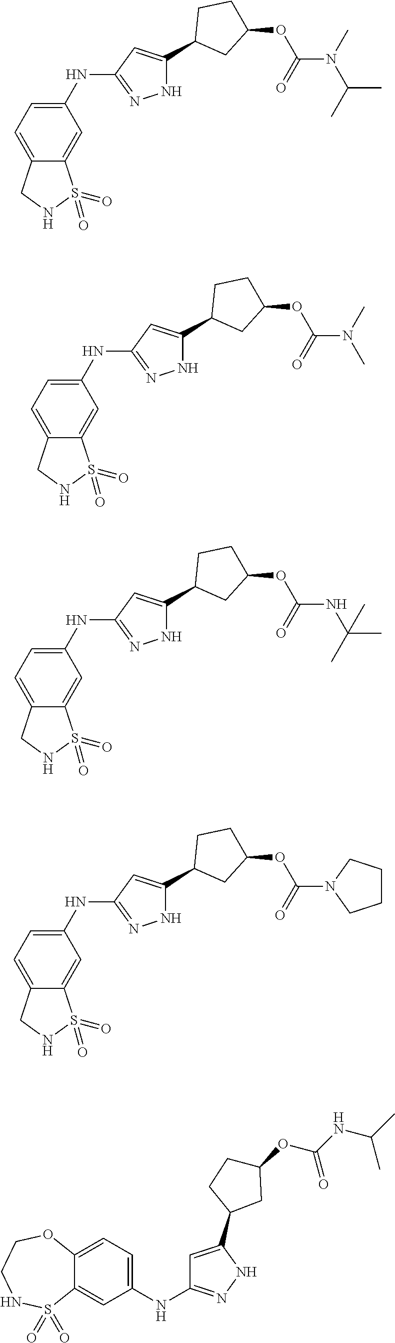 [SMILES: CC(C)N(C)C(=O)O[C@@H]1CC[C@H](c2cc(Nc3ccc4c(c3)S(=O)(=O)NC4)n[nH]2)C1 ; CN(C)C(=O)O[C@@H]1CC[C@H](c2cc(Nc3ccc4c(c3)S(=O)(=O)NC4)n[nH]2)C1 ; CC(C)(C)NC(=O)O[C@@H]1CC[C@H](c2cc(Nc3ccc4c(c3)S(=O)(=O)NC4)n[nH]2)C1 ; O=C(O[C@@H]1CC[C@H](c2cc(Nc3ccc4c(c3)S(=O)(=O)NC4)n[nH]2)C1)N1CCCC1 ; CC(C)NC(=O)O[C@@H]1CC[C@H](c2cc(Nc3ccc4c(c3)S(=O)(=O)NCCO4)n[nH]2)C1]

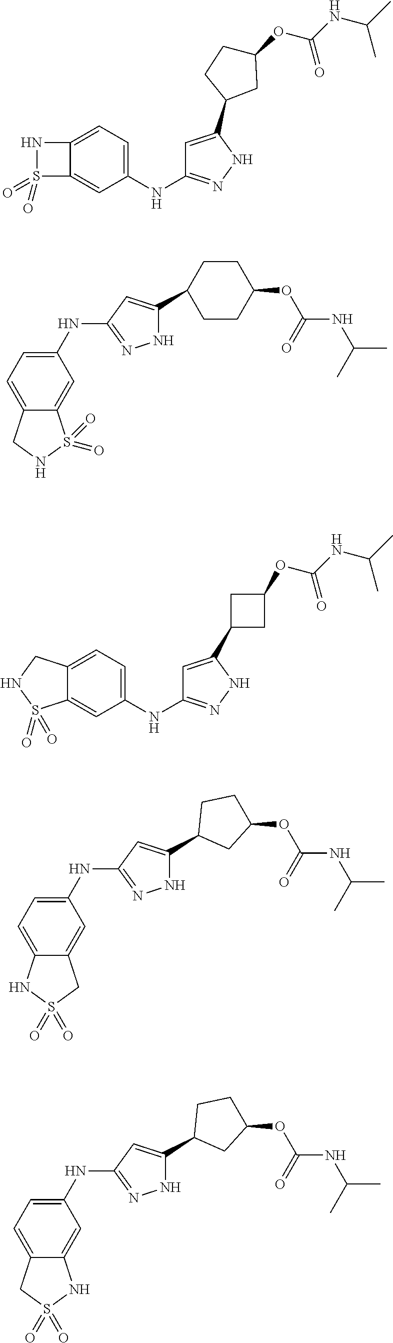 [SMILES: CC(C)NC(=O)O[C@@H]1CC[C@H](c2cc(Nc3ccc4c(c3)S(=O)(=O)N4)n[nH]2)C1 ; CC(C)NC(=O)O[C@H]1CC[C@@H](c2cc(Nc3ccc4c(c3)S(=O)(=O)NC4)n[nH]2)CC1 ; CC(C)NC(=O)O[C@H]1C[C@@H](c2cc(Nc3ccc4c(c3)S(=O)(=O)NC4)n[nH]2)C1 ; CC(C)NC(=O)O[C@@H]1CC[C@H](c2cc(Nc3ccc4c(c3)CS(=O)(=O)N4)n[nH]2)C1 ; CC(C)NC(=O)O[C@@H]1CC[C@H](c2cc(Nc3ccc4c(c3)NS(=O)(=O)C4)n[nH]2)C1]

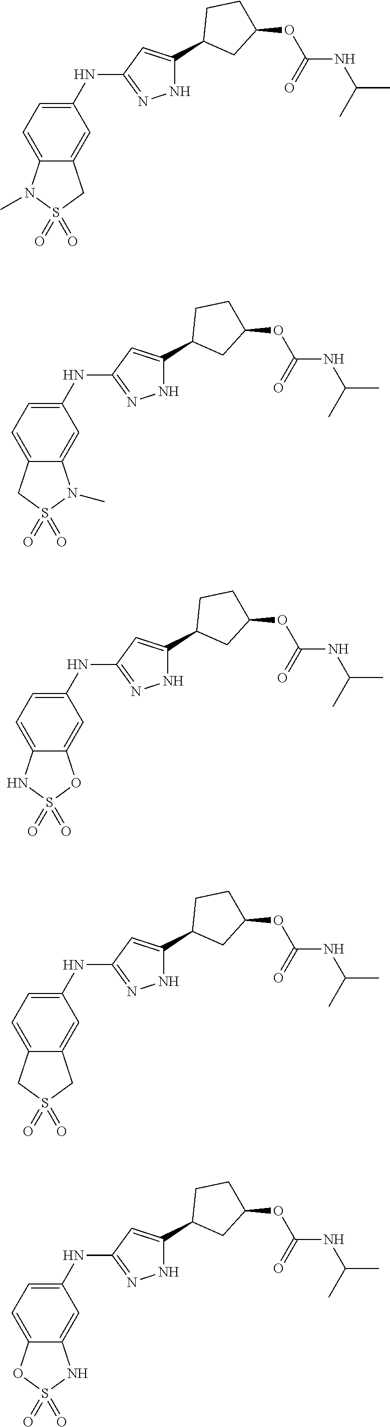 [SMILES: CC(C)NC(=O)O[C@@H]1CC[C@H](c2cc(Nc3ccc4c(c3)CS(=O)(=O)N4C)n[nH]2)C1 ; CC(C)NC(=O)O[C@@H]1CC[C@H](c2cc(Nc3ccc4c(c3)N(C)S(=O)(=O)C4)n[nH]2)C1 ; CC(C)NC(=O)O[C@@H]1CC[C@H](c2cc(Nc3ccc4c(c3)OS(=O)(=O)N4)n[nH]2)C1 ; CC(C)NC(=O)O[C@@H]1CC[C@H](c2cc(Nc3ccc4c(c3)CS(=O)(=O)C4)n[nH]2)C1 ; CC(C)NC(=O)O[C@@H]1CC[C@H](c2cc(Nc3ccc4c(c3)NS(=O)(=O)O4)n[nH]2)C1]

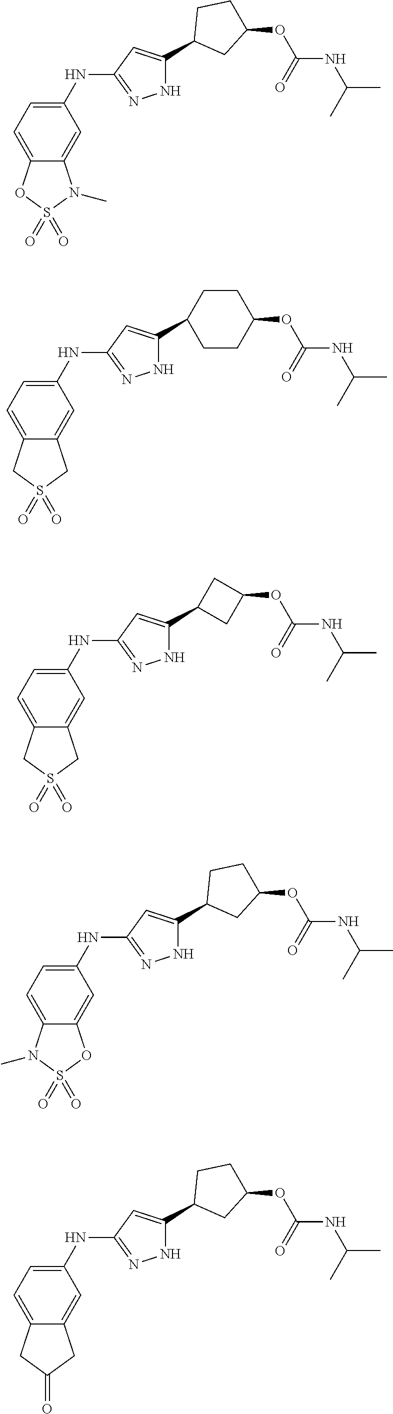

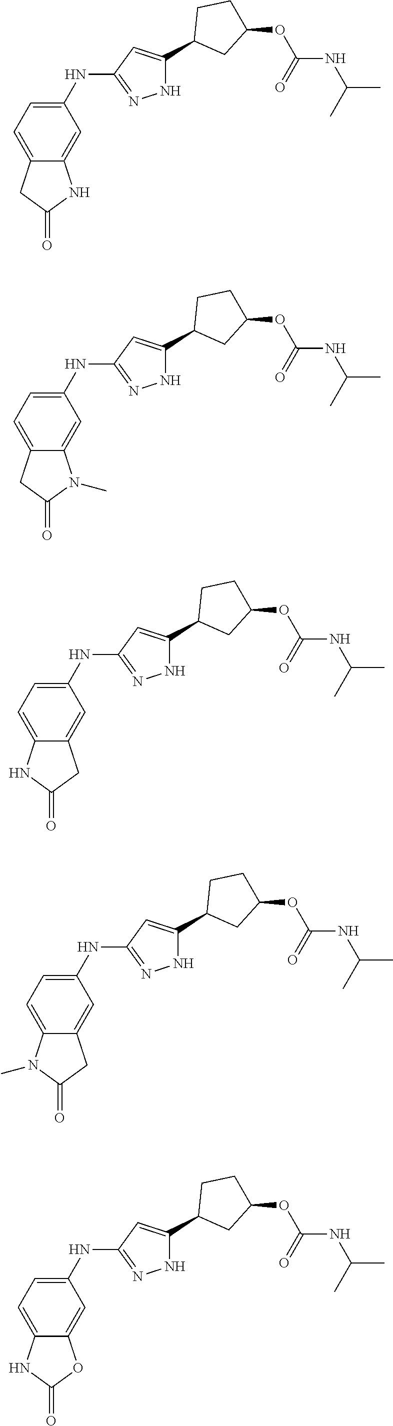

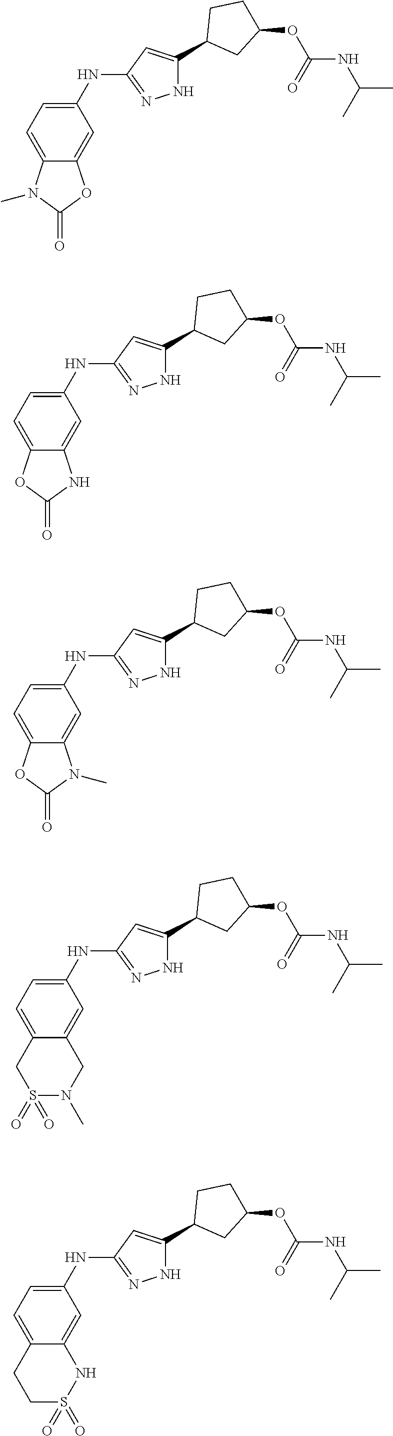 [SMILES: CC(C)NC(=O)O[C@@H]1CC[C@H](c2cc(Nc3ccc4c(c3)oc(=O)n4C)n[nH]2)C1 ; CC(C)NC(=O)O[C@@H]1CC[C@H](c2cc(Nc3ccc4oc(=O)[nH]c4c3)n[nH]2)C1 ; CC(C)NC(=O)O[C@@H]1CC[C@H](c2cc(Nc3ccc4oc(=O)n(C)c4c3)n[nH]2)C1 ; CC(C)NC(=O)O[C@@H]1CC[C@H](c2cc(Nc3ccc4c(c3)CN(C)S(=O)(=O)C4)n[nH]2)C1 ; CC(C)NC(=O)O[C@@H]1CC[C@H](c2cc(Nc3ccc4c(c3)NS(=O)(=O)CC4)n[nH]2)C1]

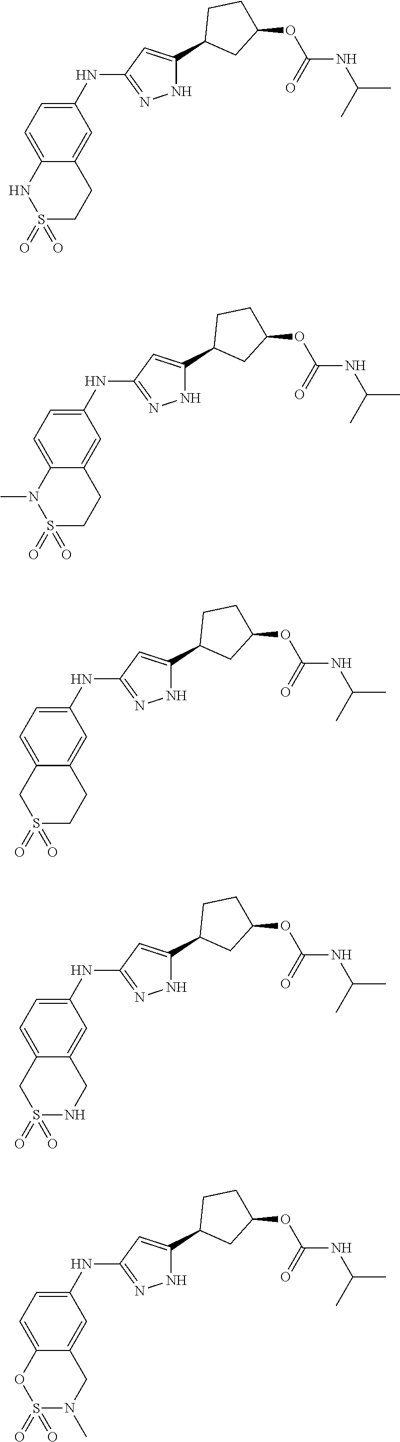

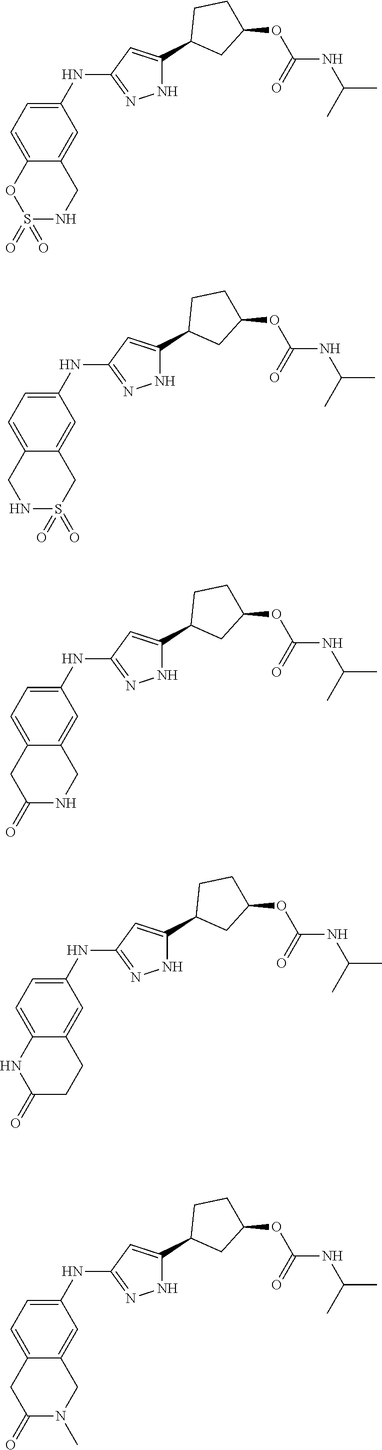 [SMILES: CC(C)NC(=O)O[C@@H]1CC[C@H](c2cc(Nc3ccc4c(c3)CNS(=O)(=O)O4)n[nH]2)C1 ; CC(C)NC(=O)O[C@@H]1CC[C@H](c2cc(Nc3ccc4c(c3)CS(=O)(=O)NC4)n[nH]2)C1 ; CC(C)NC(=O)O[C@@H]1CC[C@H](c2cc(Nc3ccc4c(c3)CNC(=O)C4)n[nH]2)C1 ; CC(C)NC(=O)O[C@@H]1CC[C@H](c2cc(Nc3ccc4c(c3)CCC(=O)N4)n[nH]2)C1 ; CC(C)NC(=O)O[C@@H]1CC[C@H](c2cc(Nc3ccc4c(c3)CN(C)C(=O)C4)n[nH]2)C1]

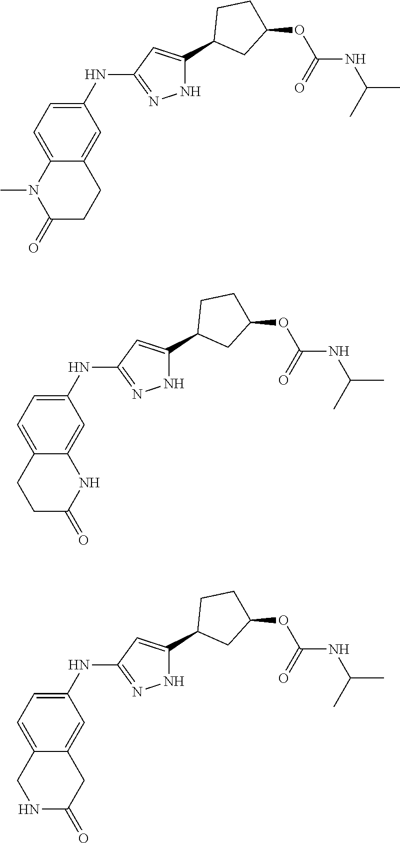 [SMILES: CC(C)NC(=O)O[C@@H]1CC[C@H](c2cc(Nc3ccc4c(c3)CCC(=O)N4C)n[nH]2)C1 ; CC(C)NC(=O)O[C@@H]1CC[C@H](c2cc(Nc3ccc4c(c3)NC(=O)CC4)n[nH]2)C1 ; CC(C)NC(=O)O[C@@H]1CC[C@H](c2cc(Nc3ccc4c(c3)CC(=O)NC4)n[nH]2)C1]

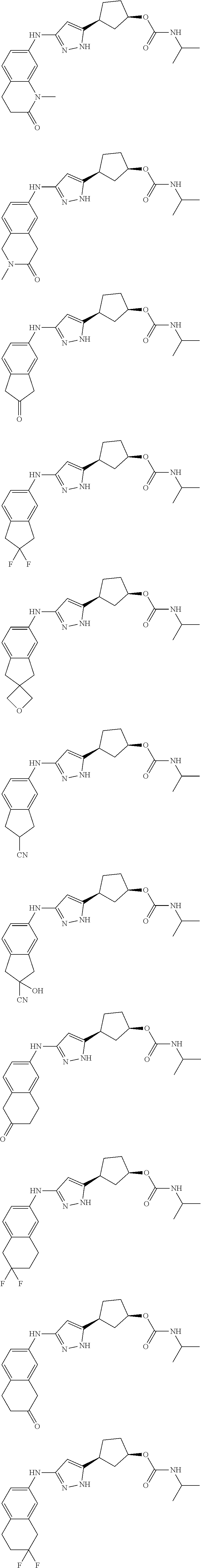

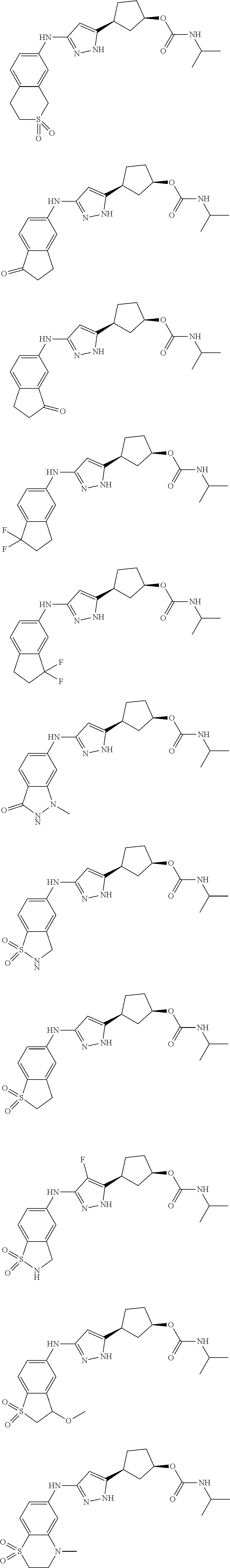

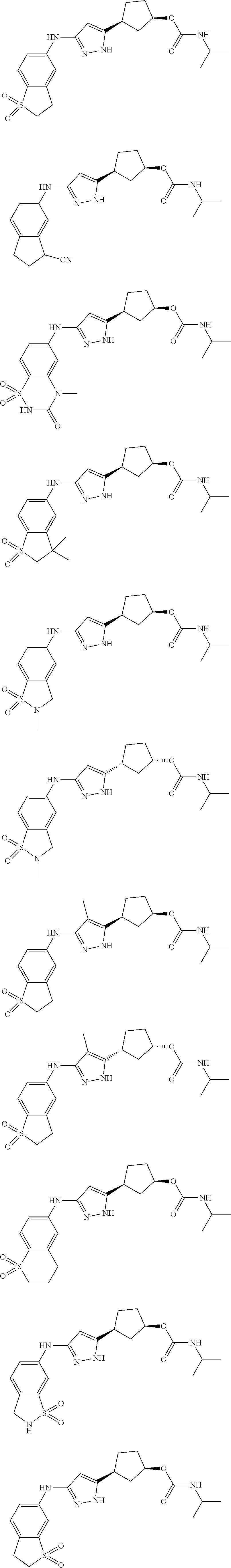

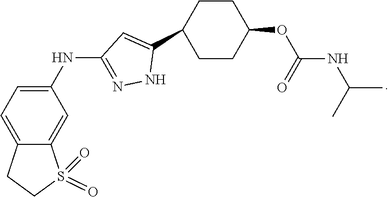 [SMILES: CC(C)NC(=O)O[C@H]1CC[C@@H](c2cc(Nc3ccc4c(c3)S(=O)(=O)CC4)n[nH]2)CC1]

Exemplary compounds of the invention include:

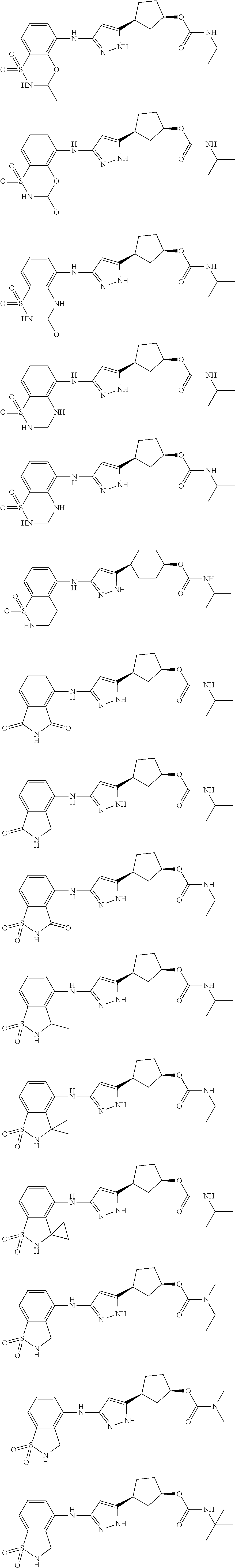

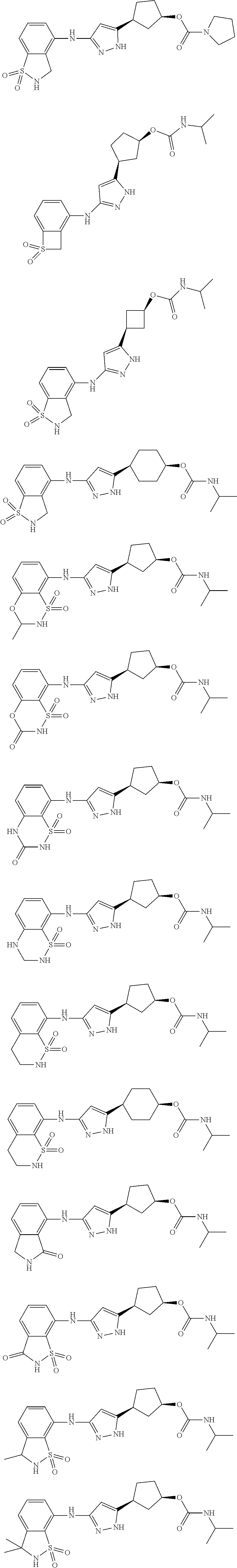

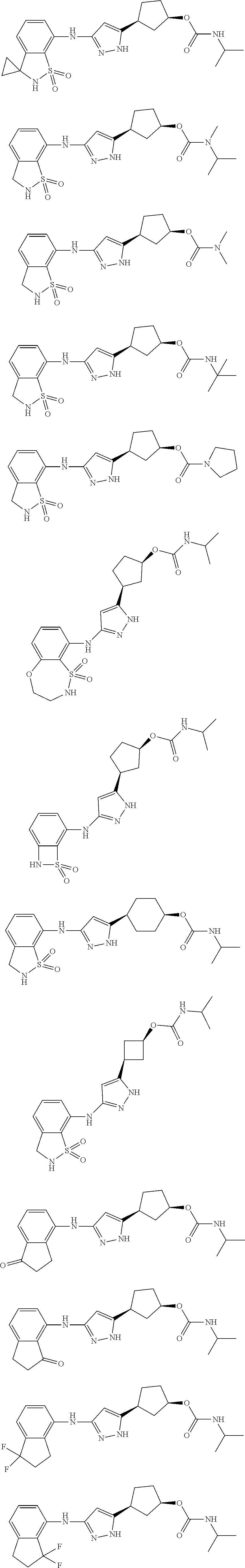

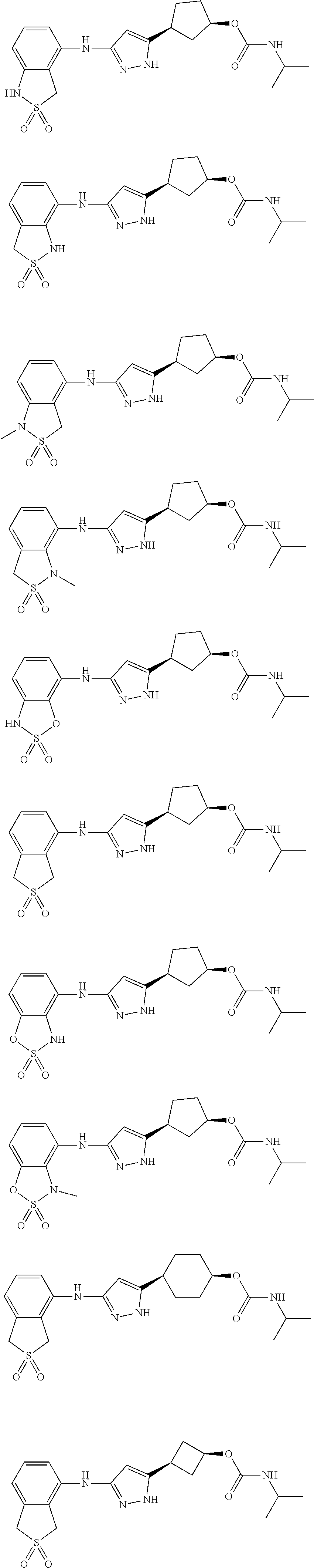

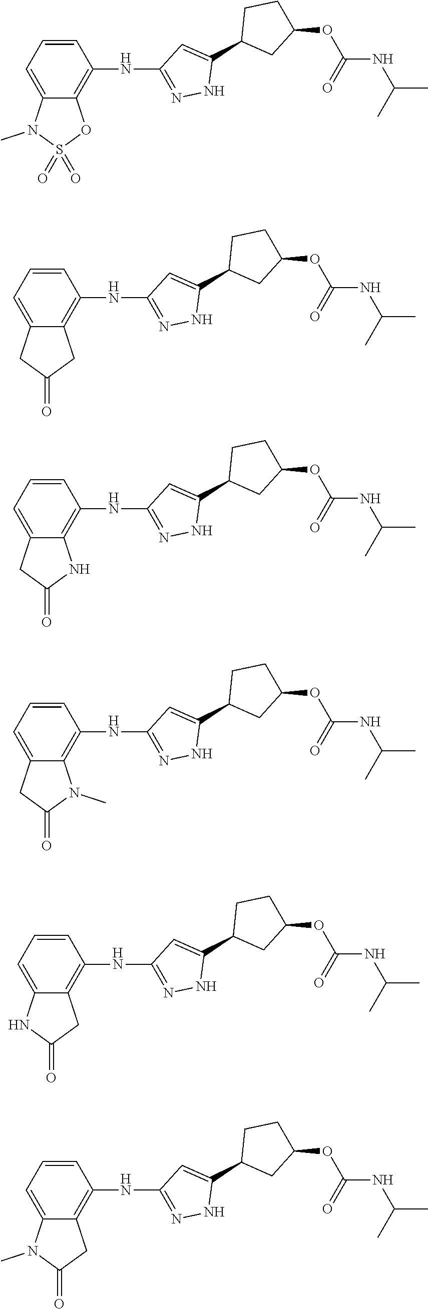

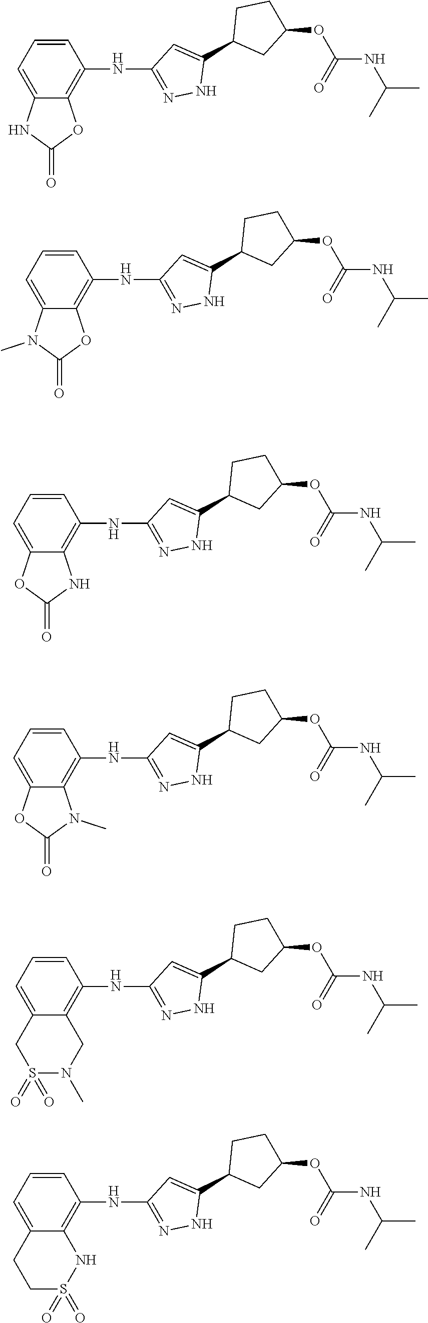

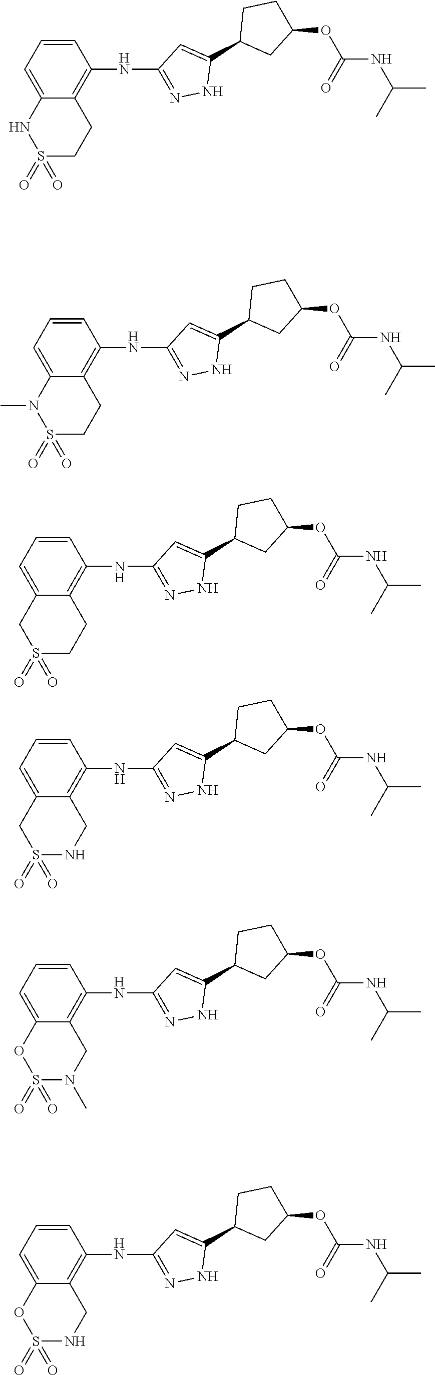

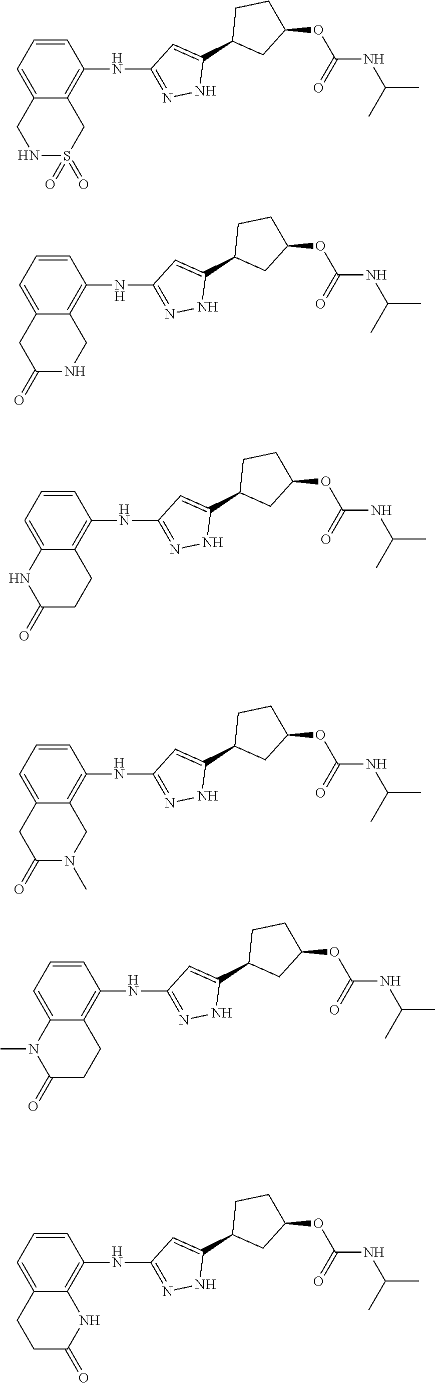

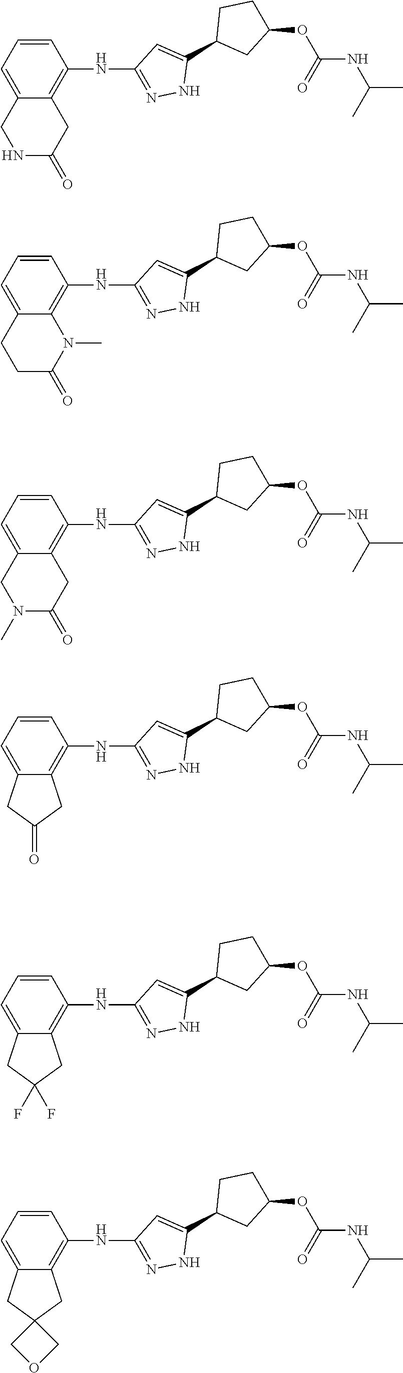 [SMILES: CC(C)NC(=O)O[C@@H]1CC[C@H](c2cc(Nc3cccc4c3CC(=O)NC4)n[nH]2)C1 ; CC(C)NC(=O)O[C@@H]1CC[C@H](c2cc(Nc3cccc4c3N(C)C(=O)CC4)n[nH]2)C1 ; CC(C)NC(=O)O[C@@H]1CC[C@H](c2cc(Nc3cccc4c3CC(=O)N(C)C4)n[nH]2)C1 ; CC(C)NC(=O)O[C@@H]1CC[C@H](c2cc(Nc3cccc4c3CC(=O)C4)n[nH]2)C1 ; CC(C)NC(=O)O[C@@H]1CC[C@H](c2cc(Nc3cccc4c3CC(F)(F)C4)n[nH]2)C1 ; CC(C)NC(=O)O[C@@H]1CC[C@H](c2cc(Nc3cccc4c3CC3(COC3)C4)n[nH]2)C1]

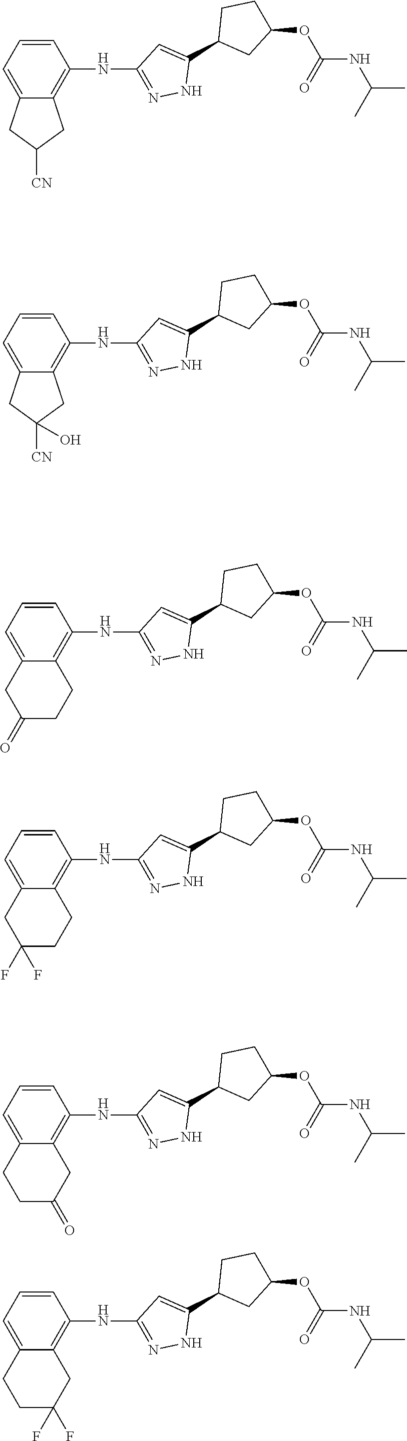 [SMILES: CC(C)NC(=O)O[C@@H]1CC[C@H](c2cc(Nc3cccc4c3CC(C#N)C4)n[nH]2)C1 ; CC(C)NC(=O)O[C@@H]1CC[C@H](c2cc(Nc3cccc4c3CC(O)(C#N)C4)n[nH]2)C1 ; CC(C)NC(=O)O[C@@H]1CC[C@H](c2cc(Nc3cccc4c3CCC(=O)C4)n[nH]2)C1 ; CC(C)NC(=O)O[C@@H]1CC[C@H](c2cc(Nc3cccc4c3CCC(F)(F)C4)n[nH]2)C1 ; CC(C)NC(=O)O[C@@H]1CC[C@H](c2cc(Nc3cccc4c3CC(=O)CC4)n[nH]2)C1 ; CC(C)NC(=O)O[C@@H]1CC[C@H](c2cc(Nc3cccc4c3CC(F)(F)CC4)n[nH]2)C1]

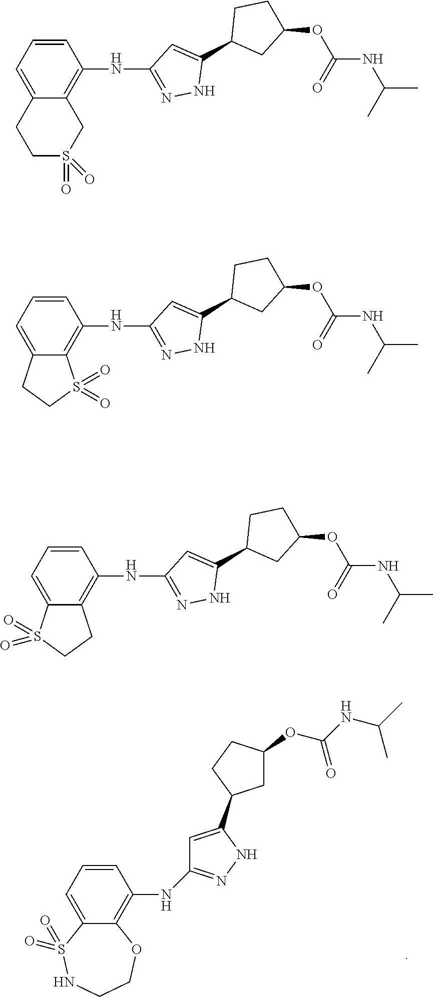 [SMILES: CC(C)NC(=O)O[C@@H]1CC[C@H](c2cc(Nc3cccc4c3CS(=O)(=O)CC4)n[nH]2)C1 ; CC(C)NC(=O)O[C@@H]1CC[C@H](c2cc(Nc3cccc4c3S(=O)(=O)CC4)n[nH]2)C1 ; CC(C)NC(=O)O[C@@H]1CC[C@H](c2cc(Nc3cccc4c3CCS4(=O)=O)n[nH]2)C1 ; CC(C)NC(=O)O[C@@H]1CC[C@H](c2cc(Nc3cccc4c3OCCNS4(=O)=O)n[nH]2)C1]

Exemplary compounds of the invention include:

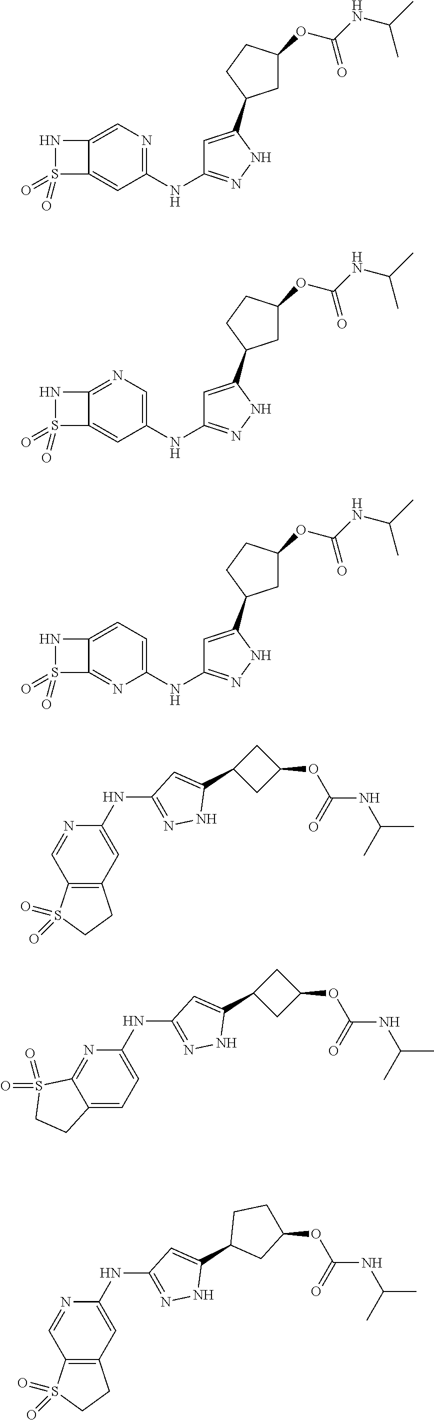 [SMILES: CC(C)NC(=O)O[C@@H]1CC[C@H](c2cc(Nc3cc4c(cn3)NS4(=O)=O)n[nH]2)C1 ; CC(C)NC(=O)O[C@@H]1CC[C@H](c2cc(Nc3cnc4c(c3)S(=O)(=O)N4)n[nH]2)C1 ; CC(C)NC(=O)O[C@@H]1CC[C@H](c2cc(Nc3ccc4c(n3)S(=O)(=O)N4)n[nH]2)C1 ; CC(C)NC(=O)O[C@H]1C[C@@H](c2cc(Nc3cc4c(cn3)S(=O)(=O)CC4)n[nH]2)C1 ; CC(C)NC(=O)O[C@H]1C[C@@H](c2cc(Nc3ccc4c(n3)S(=O)(=O)CC4)n[nH]2)C1 ; CC(C)NC(=O)O[C@@H]1CC[C@H](c2cc(Nc3cc4c(cn3)S(=O)(=O)CC4)n[nH]2)C1]

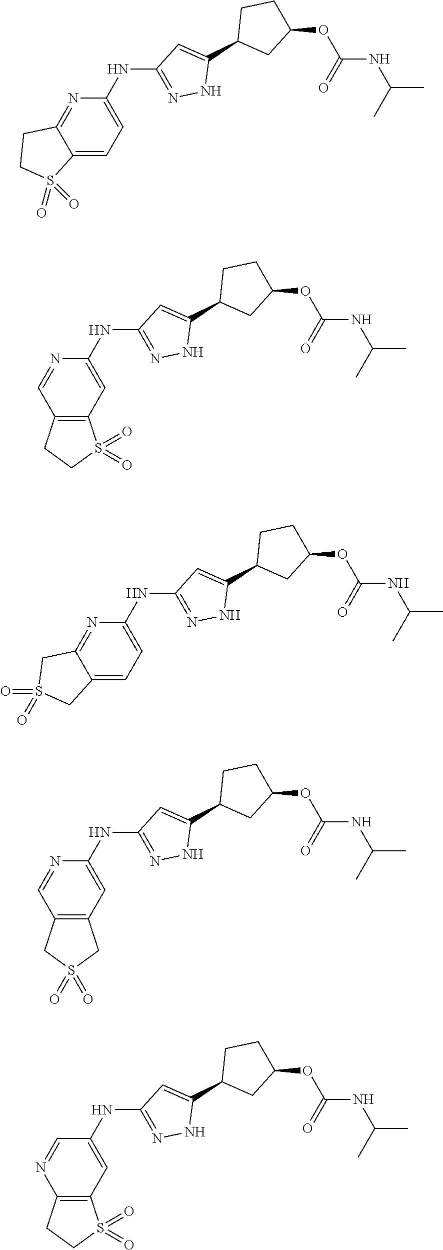 [SMILES: CC(C)NC(=O)O[C@@H]1CC[C@H](c2cc(Nc3ccc4c(n3)CCS4(=O)=O)n[nH]2)C1 ; CC(C)NC(=O)O[C@@H]1CC[C@H](c2cc(Nc3cc4c(cn3)CCS4(=O)=O)n[nH]2)C1 ; CC(C)NC(=O)O[C@@H]1CC[C@H](c2cc(Nc3ccc4c(n3)CS(=O)(=O)C4)n[nH]2)C1 ; CC(C)NC(=O)O[C@@H]1CC[C@H](c2cc(Nc3cc4c(cn3)CS(=O)(=O)C4)n[nH]2)C1 ; CC(C)NC(=O)O[C@@H]1CC[C@H](c2cc(Nc3cnc4c(c3)S(=O)(=O)CC4)n[nH]2)C1]

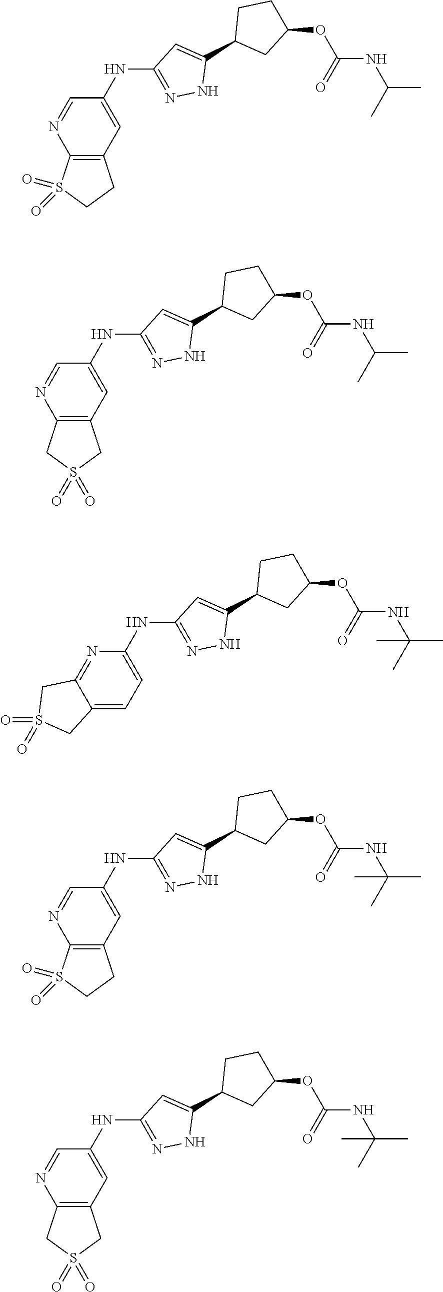 [SMILES: CC(C)NC(=O)O[C@@H]1CC[C@H](c2cc(Nc3cnc4c(c3)CCS4(=O)=O)n[nH]2)C1 ; CC(C)NC(=O)O[C@@H]1CC[C@H](c2cc(Nc3cnc4c(c3)CS(=O)(=O)C4)n[nH]2)C1 ; CC(C)(C)NC(=O)O[C@@H]1CC[C@H](c2cc(Nc3ccc4c(n3)CS(=O)(=O)C4)n[nH]2)C1 ; CC(C)(C)NC(=O)O[C@@H]1CC[C@H](c2cc(Nc3cnc4c(c3)CCS4(=O)=O)n[nH]2)C1 ; CC(C)(C)NC(=O)O[C@@H]1CC[C@H](c2cc(Nc3cnc4c(c3)CS(=O)(=O)C4)n[nH]2)C1]

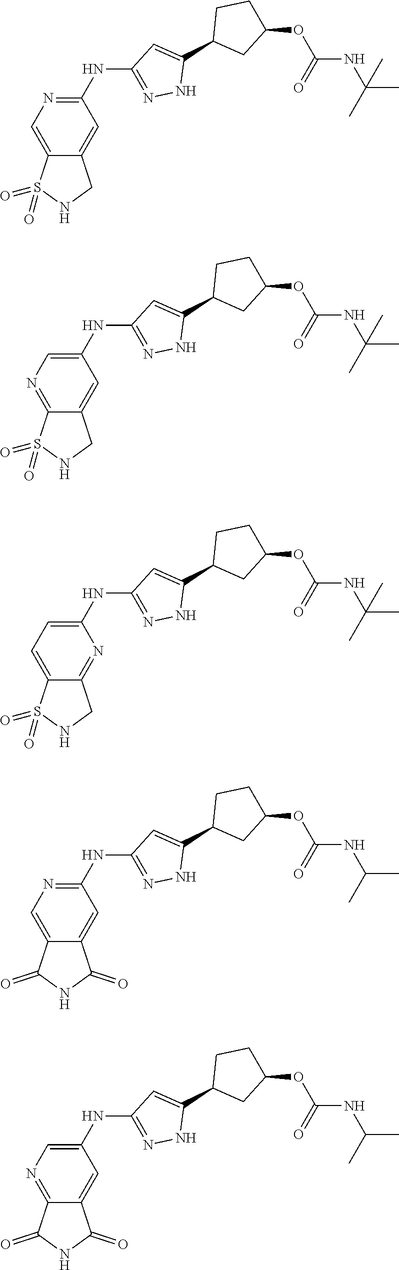 [SMILES: CC(C)(C)NC(=O)O[C@@H]1CC[C@H](c2cc(Nc3cc4c(cn3)S(=O)(=O)NC4)n[nH]2)C1 ; CC(C)(C)NC(=O)O[C@@H]1CC[C@H](c2cc(Nc3cnc4c(c3)CNS4(=O)=O)n[nH]2)C1 ; CC(C)(C)NC(=O)O[C@@H]1CC[C@H](c2cc(Nc3ccc4c(n3)CNS4(=O)=O)n[nH]2)C1 ; CC(C)NC(=O)O[C@@H]1CC[C@H](c2cc(Nc3cc4c(cn3)C(=O)NC4=O)n[nH]2)C1 ; CC(C)NC(=O)O[C@@H]1CC[C@H](c2cc(Nc3cnc4c(c3)C(=O)NC4=O)n[nH]2)C1]

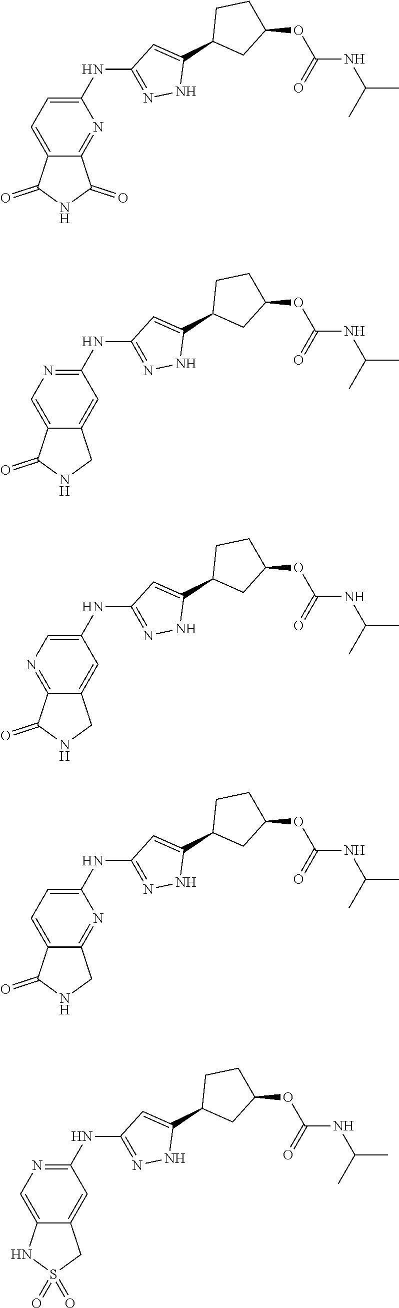 [SMILES: CC(C)NC(=O)O[C@@H]1CC[C@H](c2cc(Nc3ccc4c(n3)C(=O)NC4=O)n[nH]2)C1 ; CC(C)NC(=O)O[C@@H]1CC[C@H](c2cc(Nc3cc4c(cn3)C(=O)NC4)n[nH]2)C1 ; CC(C)NC(=O)O[C@@H]1CC[C@H](c2cc(Nc3cnc4c(c3)CNC4=O)n[nH]2)C1 ; CC(C)NC(=O)O[C@@H]1CC[C@H](c2cc(Nc3ccc4c(n3)CNC4=O)n[nH]2)C1 ; CC(C)NC(=O)O[C@@H]1CC[C@H](c2cc(Nc3cc4c(cn3)NS(=O)(=O)C4)n[nH]2)C1]

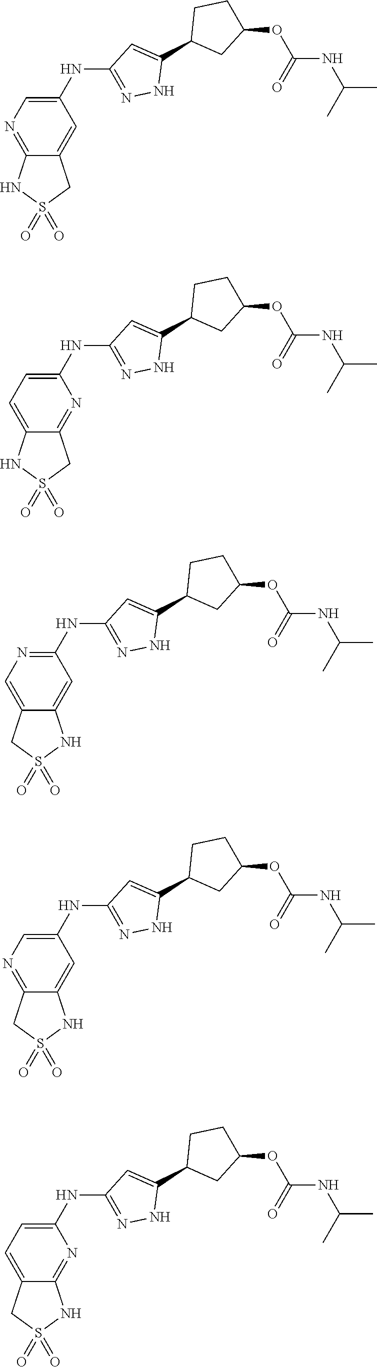 [SMILES: CC(C)NC(=O)O[C@@H]1CC[C@H](c2cc(Nc3cnc4c(c3)CS(=O)(=O)N4)n[nH]2)C1 ; CC(C)NC(=O)O[C@@H]1CC[C@H](c2cc(Nc3ccc4c(n3)CS(=O)(=O)N4)n[nH]2)C1 ; CC(C)NC(=O)O[C@@H]1CC[C@H](c2cc(Nc3cc4c(cn3)CS(=O)(=O)N4)n[nH]2)C1 ; CC(C)NC(=O)O[C@@H]1CC[C@H](c2cc(Nc3cnc4c(c3)NS(=O)(=O)C4)n[nH]2)C1 ; CC(C)NC(=O)O[C@@H]1CC[C@H](c2cc(Nc3ccc4c(n3)NS(=O)(=O)C4)n[nH]2)C1]

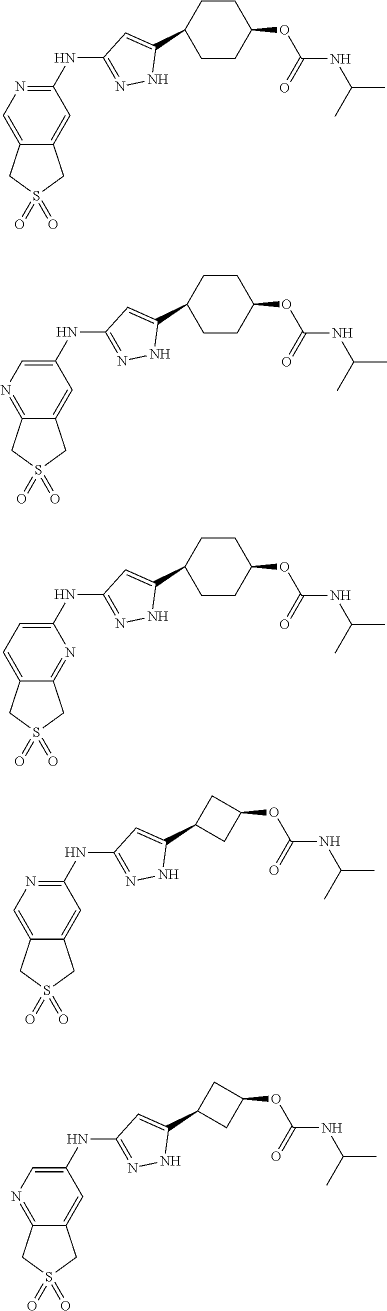 [SMILES: CC(C)NC(=O)O[C@H]1CC[C@@H](c2cc(Nc3cc4c(cn3)CS(=O)(=O)C4)n[nH]2)CC1 ; CC(C)NC(=O)O[C@H]1CC[C@@H](c2cc(Nc3cnc4c(c3)CS(=O)(=O)C4)n[nH]2)CC1 ; CC(C)NC(=O)O[C@H]1CC[C@@H](c2cc(Nc3ccc4c(n3)CS(=O)(=O)C4)n[nH]2)CC1 ; CC(C)NC(=O)O[C@H]1C[C@@H](c2cc(Nc3cc4c(cn3)CS(=O)(=O)C4)n[nH]2)C1 ; CC(C)NC(=O)O[C@H]1C[C@@H](c2cc(Nc3cnc4c(c3)CS(=O)(=O)C4)n[nH]2)C1]

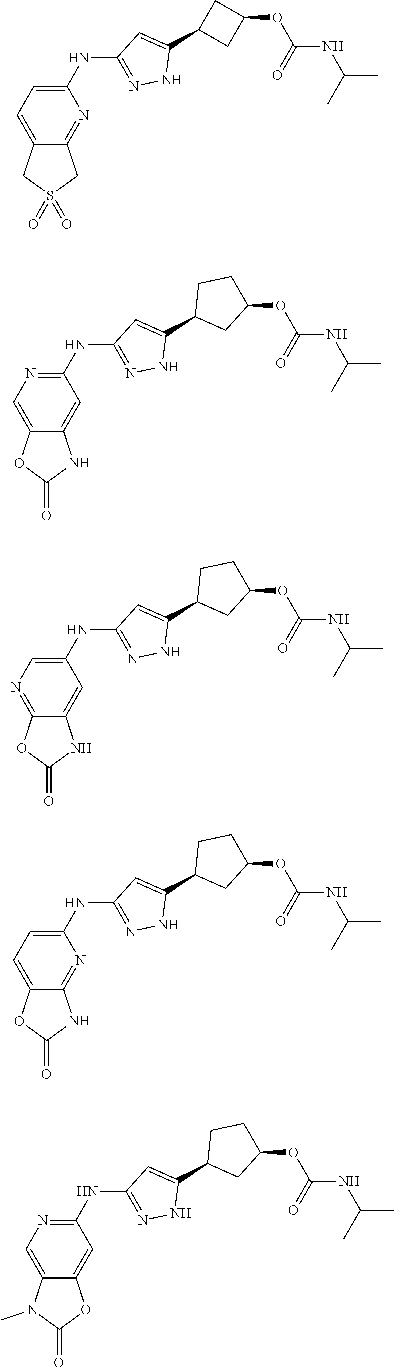

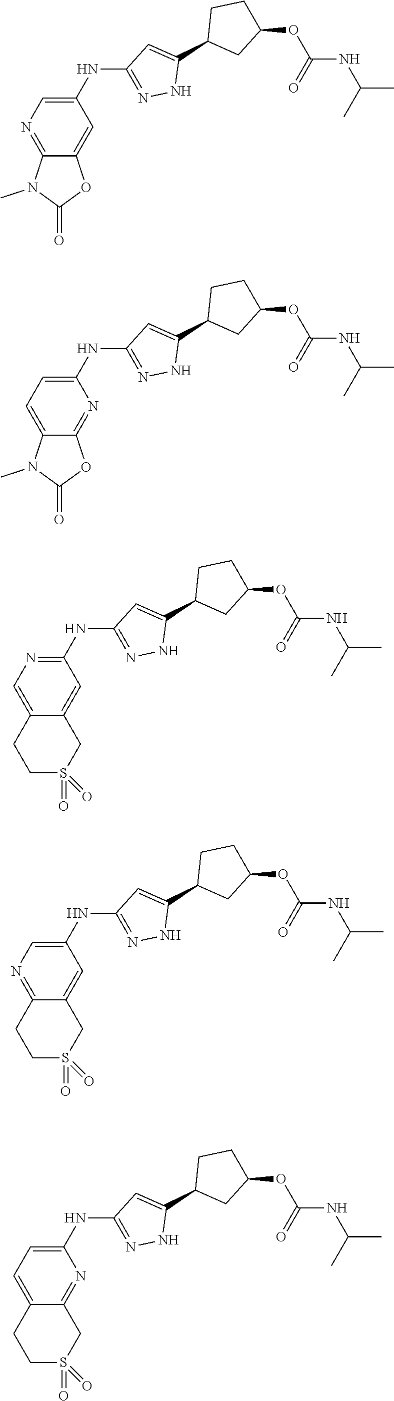

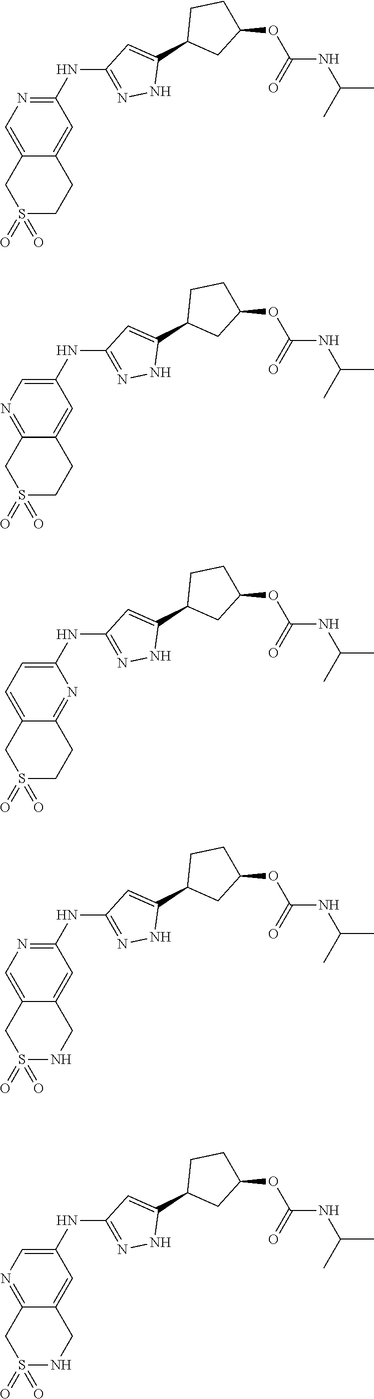 [SMILES: CC(C)NC(=O)O[C@@H]1CC[C@H](c2cc(Nc3cc4c(cn3)CS(=O)(=O)CC4)n[nH]2)C1 ; CC(C)NC(=O)O[C@@H]1CC[C@H](c2cc(Nc3cnc4c(c3)CCS(=O)(=O)C4)n[nH]2)C1 ; CC(C)NC(=O)O[C@@H]1CC[C@H](c2cc(Nc3ccc4c(n3)CCS(=O)(=O)C4)n[nH]2)C1 ; CC(C)NC(=O)O[C@@H]1CC[C@H](c2cc(Nc3cc4c(cn3)CS(=O)(=O)NC4)n[nH]2)C1 ; CC(C)NC(=O)O[C@@H]1CC[C@H](c2cc(Nc3cnc4c(c3)CNS(=O)(=O)C4)n[nH]2)C1]

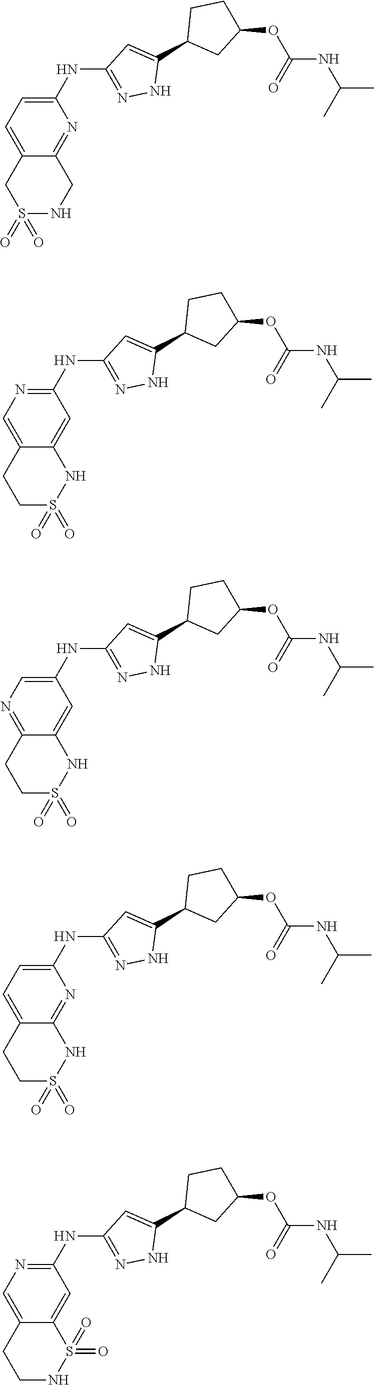 [SMILES: CC(C)NC(=O)O[C@@H]1CC[C@H](c2cc(Nc3ccc4c(n3)CNS(=O)(=O)C4)n[nH]2)C1 ; CC(C)NC(=O)O[C@@H]1CC[C@H](c2cc(Nc3cc4c(cn3)CCS(=O)(=O)N4)n[nH]2)C1 ; CC(C)NC(=O)O[C@@H]1CC[C@H](c2cc(Nc3cnc4c(c3)NS(=O)(=O)CC4)n[nH]2)C1 ; CC(C)NC(=O)O[C@@H]1CC[C@H](c2cc(Nc3ccc4c(n3)NS(=O)(=O)CC4)n[nH]2)C1 ; CC(C)NC(=O)O[C@@H]1CC[C@H](c2cc(Nc3cc4c(cn3)CCNS4(=O)=O)n[nH]2)C1]

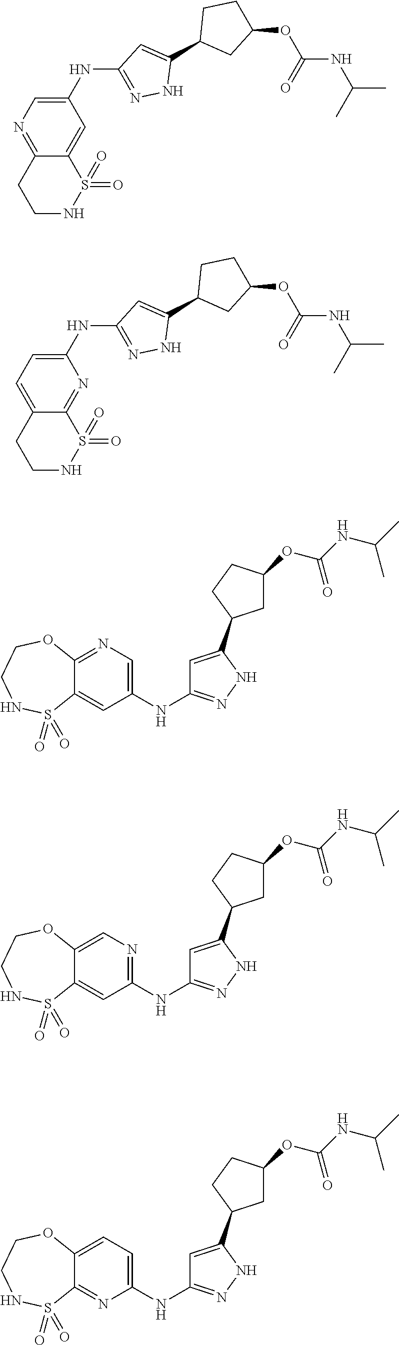 [SMILES: CC(C)NC(=O)O[C@@H]1CC[C@H](c2cc(Nc3cnc4c(c3)S(=O)(=O)NCC4)n[nH]2)C1 ; CC(C)NC(=O)O[C@@H]1CC[C@H](c2cc(Nc3ccc4c(n3)S(=O)(=O)NCC4)n[nH]2)C1 ; CC(C)NC(=O)O[C@@H]1CC[C@H](c2cc(Nc3cnc4c(c3)S(=O)(=O)NCCO4)n[nH]2)C1 ; CC(C)NC(=O)O[C@@H]1CC[C@H](c2cc(Nc3cc4c(cn3)OCCNS4(=O)=O)n[nH]2)C1 ; CC(C)NC(=O)O[C@@H]1CC[C@H](c2cc(Nc3ccc4c(n3)S(=O)(=O)NCCO4)n[nH]2)C1]

Exemplary compounds of the invention include:

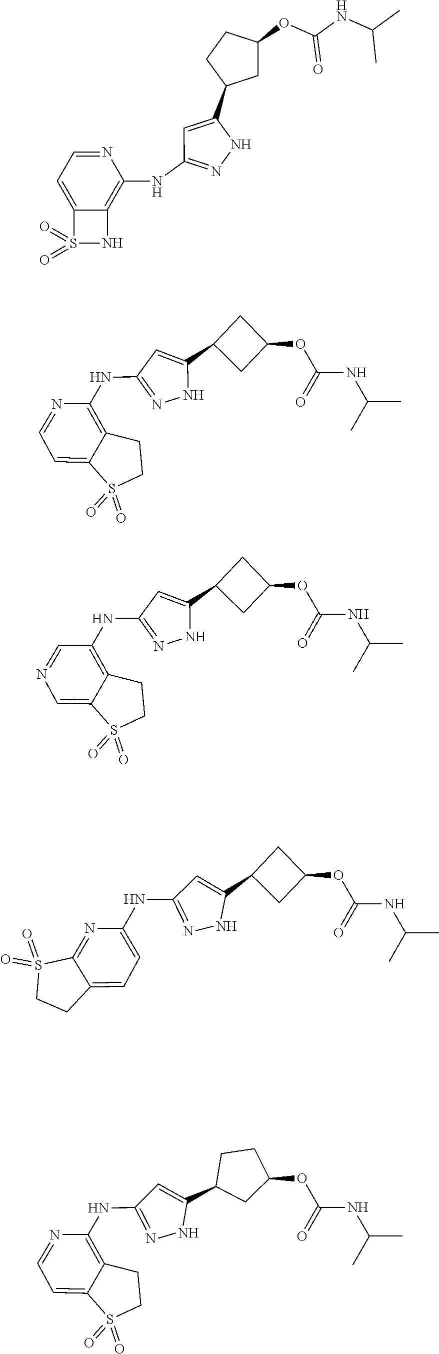 [SMILES: CC(C)NC(=O)O[C@@H]1CC[C@H](c2cc(Nc3nccc4c3NS4(=O)=O)n[nH]2)C1 ; CC(C)NC(=O)O[C@H]1C[C@@H](c2cc(Nc3nccc4c3CCS4(=O)=O)n[nH]2)C1 ; CC(C)NC(=O)O[C@H]1C[C@@H](c2cc(Nc3cncc4c3CCS4(=O)=O)n[nH]2)C1 ; CC(C)NC(=O)O[C@H]1C[C@@H](c2cc(Nc3ccc4c(n3)S(=O)(=O)CC4)n[nH]2)C1 ; CC(C)NC(=O)O[C@@H]1CC[C@H](c2cc(Nc3nccc4c3CCS4(=O)=O)n[nH]2)C1]

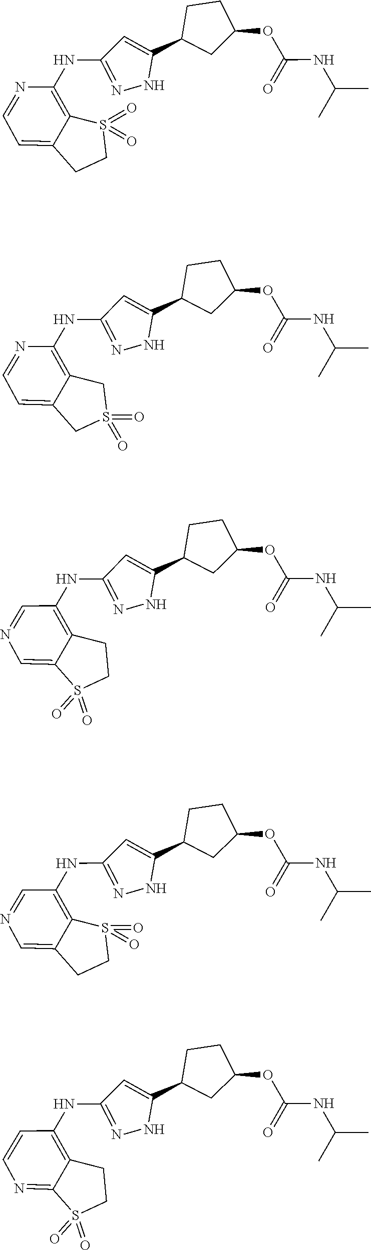 [SMILES: CC(C)NC(=O)O[C@@H]1CC[C@H](c2cc(Nc3nccc4c3S(=O)(=O)CC4)n[nH]2)C1 ; CC(C)NC(=O)O[C@@H]1CC[C@H](c2cc(Nc3nccc4c3CS(=O)(=O)C4)n[nH]2)C1 ; CC(C)NC(=O)O[C@@H]1CC[C@H](c2cc(Nc3cncc4c3CCS4(=O)=O)n[nH]2)C1 ; CC(C)NC(=O)O[C@@H]1CC[C@H](c2cc(Nc3cncc4c3S(=O)(=O)CC4)n[nH]2)C1 ; CC(C)NC(=O)O[C@@H]1CC[C@H](c2cc(Nc3ccnc4c3CCS4(=O)=O)n[nH]2)C1]

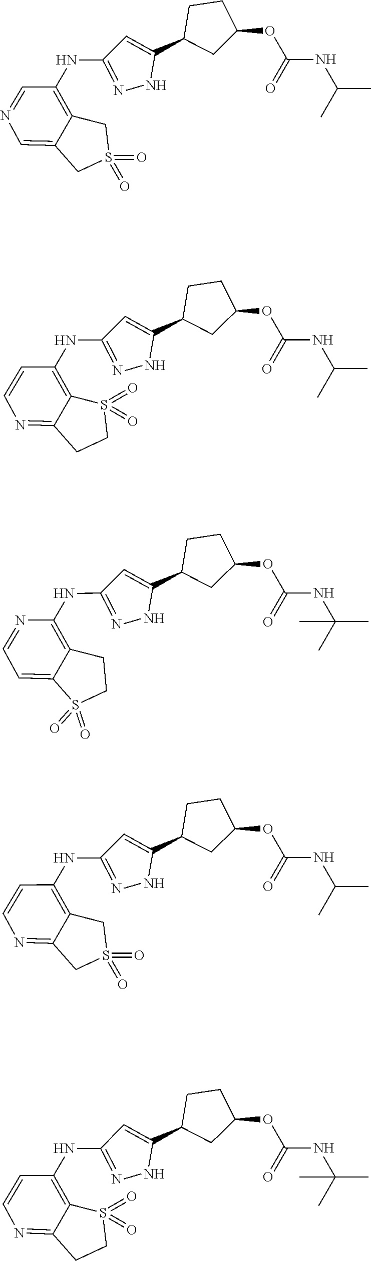 [SMILES: CC(C)NC(=O)O[C@@H]1CC[C@H](c2cc(Nc3cncc4c3CS(=O)(=O)C4)n[nH]2)C1 ; CC(C)NC(=O)O[C@@H]1CC[C@H](c2cc(Nc3ccnc4c3S(=O)(=O)CC4)n[nH]2)C1 ; CC(C)(C)NC(=O)O[C@@H]1CC[C@H](c2cc(Nc3nccc4c3CCS4(=O)=O)n[nH]2)C1 ; CC(C)NC(=O)O[C@@H]1CC[C@H](c2cc(Nc3ccnc4c3CS(=O)(=O)C4)n[nH]2)C1 ; CC(C)(C)NC(=O)O[C@@H]1CC[C@H](c2cc(Nc3ccnc4c3S(=O)(=O)CC4)n[nH]2)C1]

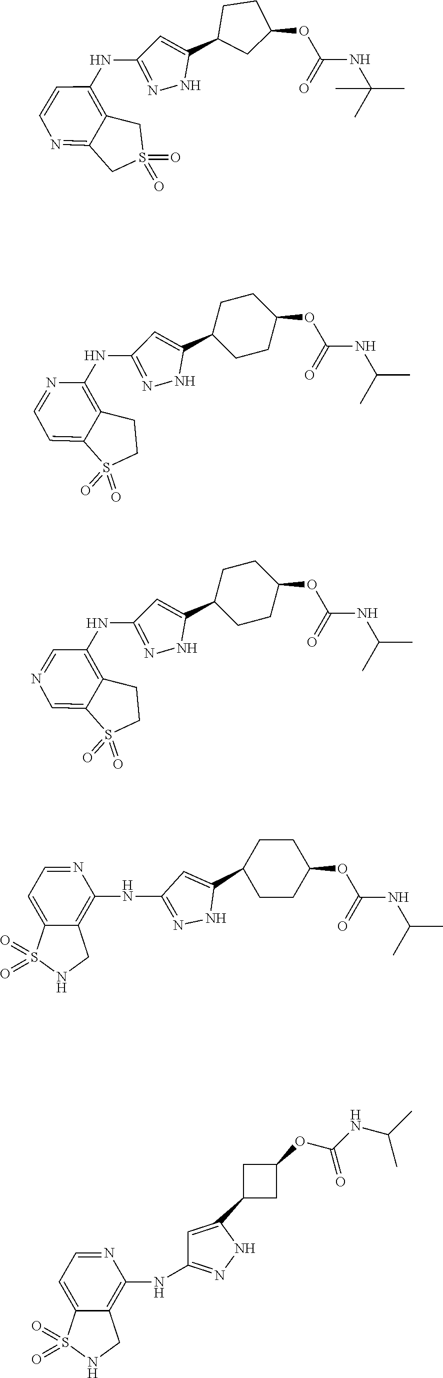 [SMILES: CC(C)(C)NC(=O)O[C@@H]1CC[C@H](c2cc(Nc3ccnc4c3CS(=O)(=O)C4)n[nH]2)C1 ; CC(C)NC(=O)O[C@H]1CC[C@@H](c2cc(Nc3nccc4c3CCS4(=O)=O)n[nH]2)CC1 ; CC(C)NC(=O)O[C@H]1CC[C@@H](c2cc(Nc3cncc4c3CCS4(=O)=O)n[nH]2)CC1 ; CC(C)NC(=O)O[C@H]1CC[C@@H](c2cc(Nc3nccc4c3CNS4(=O)=O)n[nH]2)CC1 ; CC(C)NC(=O)O[C@H]1C[C@@H](c2cc(Nc3nccc4c3CNS4(=O)=O)n[nH]2)C1]

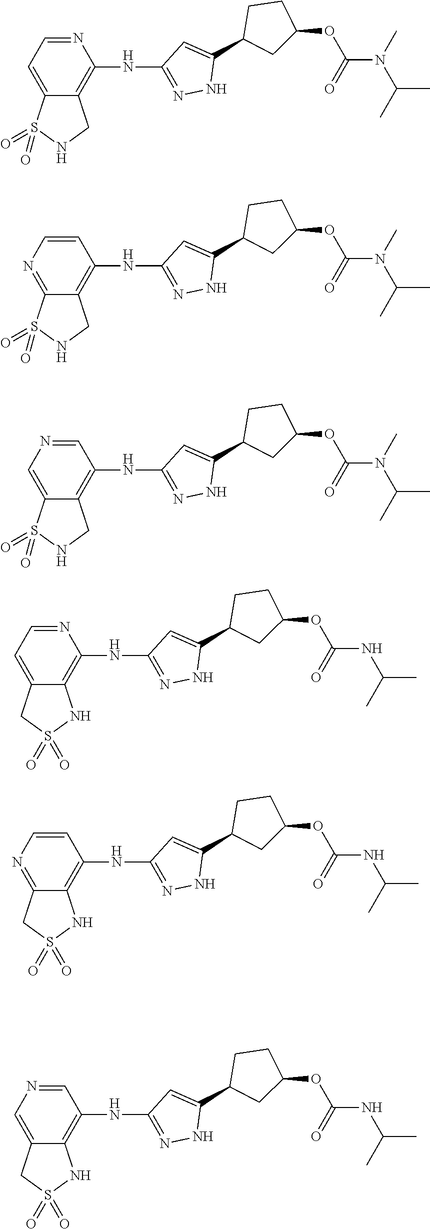 [SMILES: CC(C)N(C)C(=O)O[C@@H]1CC[C@H](c2cc(Nc3nccc4c3CNS4(=O)=O)n[nH]2)C1 ; CC(C)N(C)C(=O)O[C@@H]1CC[C@H](c2cc(Nc3ccnc4c3CNS4(=O)=O)n[nH]2)C1 ; CC(C)N(C)C(=O)O[C@@H]1CC[C@H](c2cc(Nc3cncc4c3CNS4(=O)=O)n[nH]2)C1 ; CC(C)NC(=O)O[C@@H]1CC[C@H](c2cc(Nc3nccc4c3NS(=O)(=O)C4)n[nH]2)C1 ; CC(C)NC(=O)O[C@@H]1CC[C@H](c2cc(Nc3ccnc4c3NS(=O)(=O)C4)n[nH]2)C1 ; CC(C)NC(=O)O[C@@H]1CC[C@H](c2cc(Nc3cncc4c3NS(=O)(=O)C4)n[nH]2)C1]

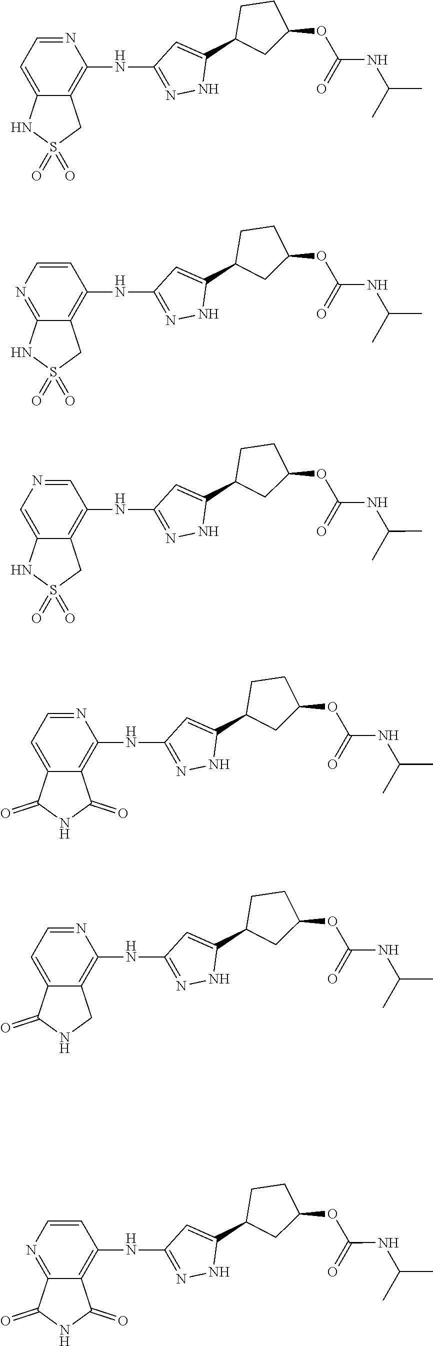 [SMILES: CC(C)NC(=O)O[C@@H]1CC[C@H](c2cc(Nc3nccc4c3CS(=O)(=O)N4)n[nH]2)C1 ; CC(C)NC(=O)O[C@@H]1CC[C@H](c2cc(Nc3ccnc4c3CS(=O)(=O)N4)n[nH]2)C1 ; CC(C)NC(=O)O[C@@H]1CC[C@H](c2cc(Nc3cncc4c3CS(=O)(=O)N4)n[nH]2)C1 ; CC(C)NC(=O)O[C@@H]1CC[C@H](c2cc(Nc3nccc4c3C(=O)NC4=O)n[nH]2)C1 ; CC(C)NC(=O)O[C@@H]1CC[C@H](c2cc(Nc3nccc4c3CNC4=O)n[nH]2)C1 ; CC(C)NC(=O)O[C@@H]1CC[C@H](c2cc(Nc3ccnc4c3C(=O)NC4=O)n[nH]2)C1]

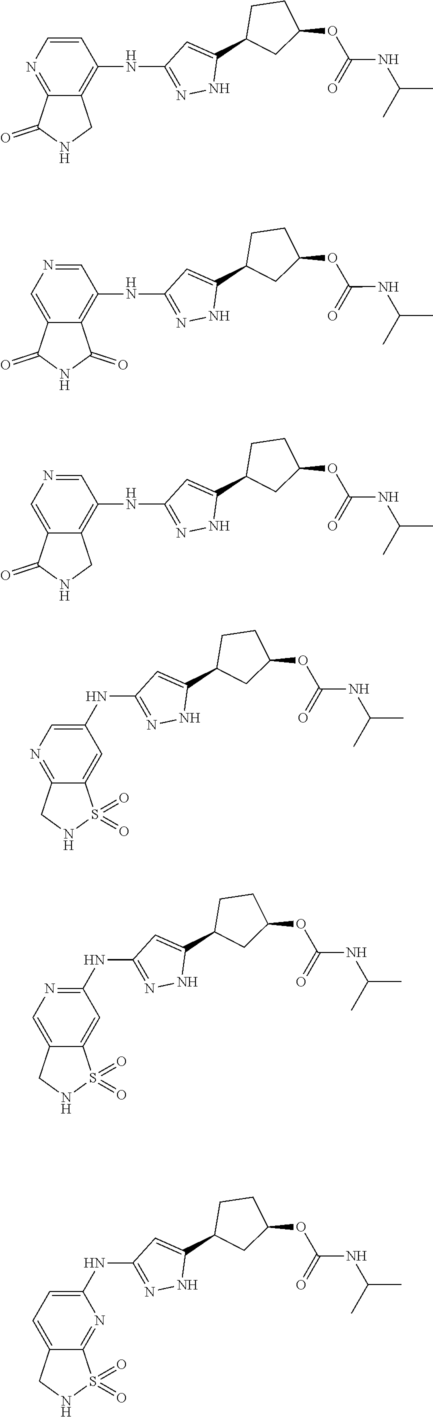 [SMILES: CC(C)NC(=O)O[C@@H]1CC[C@H](c2cc(Nc3ccnc4c3CNC4=O)n[nH]2)C1 ; CC(C)NC(=O)O[C@@H]1CC[C@H](c2cc(Nc3cncc4c3C(=O)NC4=O)n[nH]2)C1 ; CC(C)NC(=O)O[C@@H]1CC[C@H](c2cc(Nc3cncc4c3CNC4=O)n[nH]2)C1 ; CC(C)NC(=O)O[C@@H]1CC[C@H](c2cc(Nc3cnc4c(c3)S(=O)(=O)NC4)n[nH]2)C1 ; CC(C)NC(=O)O[C@@H]1CC[C@H](c2cc(Nc3cc4c(cn3)CNS4(=O)=O)n[nH]2)C1 ; CC(C)NC(=O)O[C@@H]1CC[C@H](c2cc(Nc3ccc4c(n3)S(=O)(=O)NC4)n[nH]2)C1]

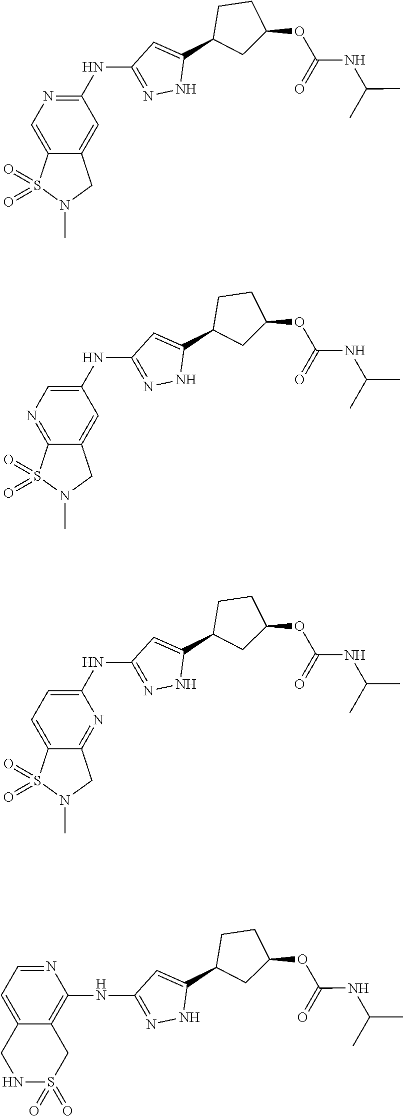 [SMILES: CC(C)NC(=O)O[C@@H]1CC[C@H](c2cc(Nc3cc4c(cn3)S(=O)(=O)N(C)C4)n[nH]2)C1 ; CC(C)NC(=O)O[C@@H]1CC[C@H](c2cc(Nc3cnc4c(c3)CN(C)S4(=O)=O)n[nH]2)C1 ; CC(C)NC(=O)O[C@@H]1CC[C@H](c2cc(Nc3ccc4c(n3)CN(C)S4(=O)=O)n[nH]2)C1 ; CC(C)NC(=O)O[C@@H]1CC[C@H](c2cc(Nc3nccc4c3CS(=O)(=O)NC4)n[nH]2)C1]

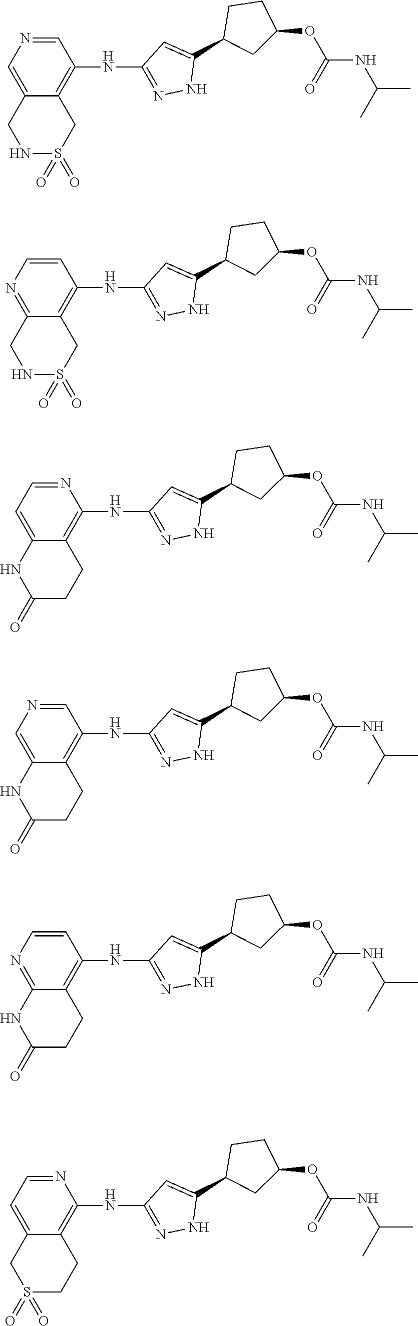 [SMILES: CC(C)NC(=O)O[C@@H]1CC[C@H](c2cc(Nc3cncc4c3CS(=O)(=O)NC4)n[nH]2)C1 ; CC(C)NC(=O)O[C@@H]1CC[C@H](c2cc(Nc3ccnc4c3CS(=O)(=O)NC4)n[nH]2)C1 ; CC(C)NC(=O)O[C@@H]1CC[C@H](c2cc(Nc3nccc4c3CCC(=O)N4)n[nH]2)C1 ; CC(C)NC(=O)O[C@@H]1CC[C@H](c2cc(Nc3cncc4c3CCC(=O)N4)n[nH]2)C1 ; CC(C)NC(=O)O[C@@H]1CC[C@H](c2cc(Nc3ccnc4c3CCC(=O)N4)n[nH]2)C1 ; CC(C)NC(=O)O[C@@H]1CC[C@H](c2cc(Nc3nccc4c3CCS(=O)(=O)C4)n[nH]2)C1]

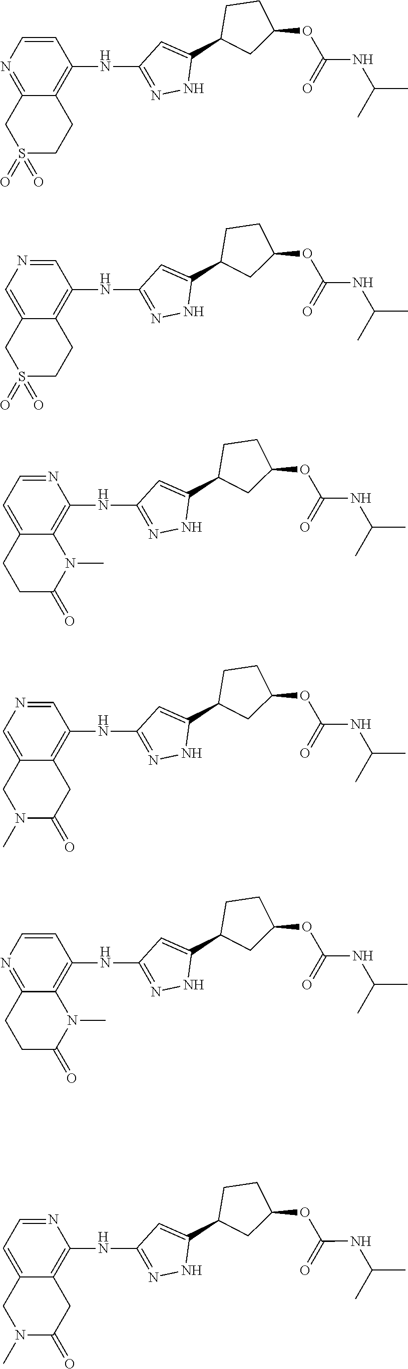 [SMILES: CC(C)NC(=O)O[C@@H]1CC[C@H](c2cc(Nc3ccnc4c3CCS(=O)(=O)C4)n[nH]2)C1 ; CC(C)NC(=O)O[C@@H]1CC[C@H](c2cc(Nc3cncc4c3CCS(=O)(=O)C4)n[nH]2)C1 ; CC(C)NC(=O)O[C@@H]1CC[C@H](c2cc(Nc3nccc4c3N(C)C(=O)CC4)n[nH]2)C1 ; CC(C)NC(=O)O[C@@H]1CC[C@H](c2cc(Nc3cncc4c3CC(=O)N(C)C4)n[nH]2)C1 ; CC(C)NC(=O)O[C@@H]1CC[C@H](c2cc(Nc3ccnc4c3N(C)C(=O)CC4)n[nH]2)C1 ; CC(C)NC(=O)O[C@@H]1CC[C@H](c2cc(Nc3nccc4c3CC(=O)N(C)C4)n[nH]2)C1]

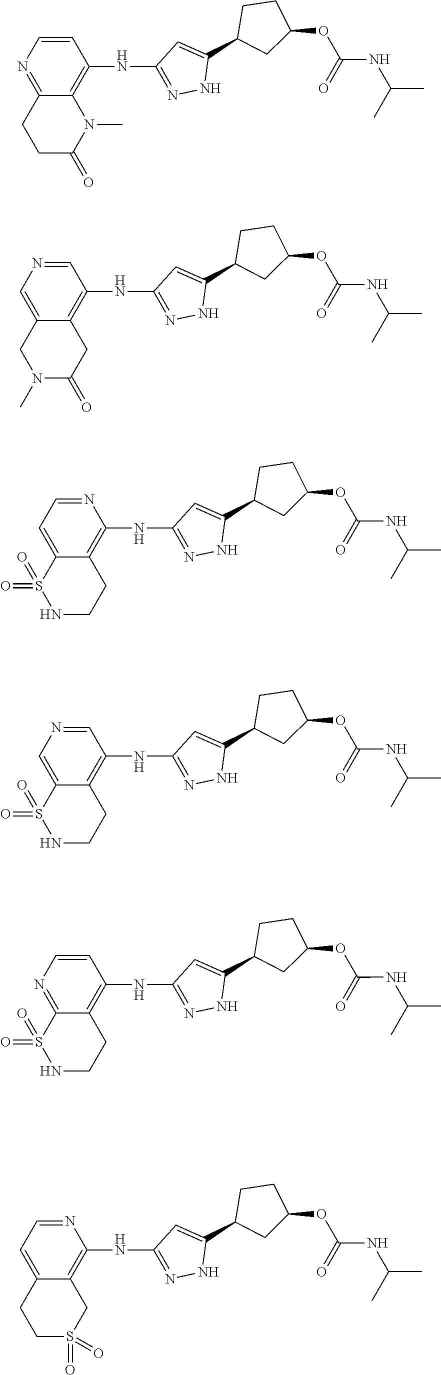 [SMILES: CC(C)NC(=O)O[C@@H]1CC[C@H](c2cc(Nc3ccnc4c3N(C)C(=O)CC4)n[nH]2)C1 ; CC(C)NC(=O)O[C@@H]1CC[C@H](c2cc(Nc3cncc4c3CC(=O)N(C)C4)n[nH]2)C1 ; CC(C)NC(=O)O[C@@H]1CC[C@H](c2cc(Nc3nccc4c3CCNS4(=O)=O)n[nH]2)C1 ; CC(C)NC(=O)O[C@@H]1CC[C@H](c2cc(Nc3cncc4c3CCNS4(=O)=O)n[nH]2)C1 ; CC(C)NC(=O)O[C@@H]1CC[C@H](c2cc(Nc3ccnc4c3CCNS4(=O)=O)n[nH]2)C1 ; CC(C)NC(=O)O[C@@H]1CC[C@H](c2cc(Nc3nccc4c3CS(=O)(=O)CC4)n[nH]2)C1]

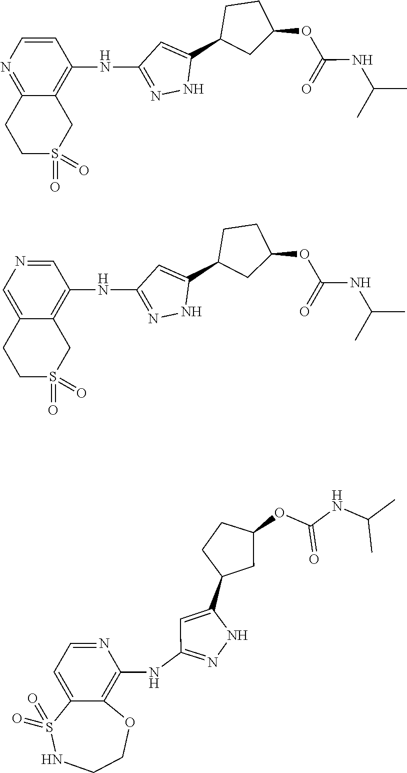 [SMILES: CC(C)NC(=O)O[C@@H]1CC[C@H](c2cc(Nc3ccnc4c3CS(=O)(=O)CC4)n[nH]2)C1 ; CC(C)NC(=O)O[C@@H]1CC[C@H](c2cc(Nc3cncc4c3CS(=O)(=O)CC4)n[nH]2)C1 ; CC(C)NC(=O)O[C@@H]1CC[C@H](c2cc(Nc3nccc4c3OCCNS4(=O)=O)n[nH]2)C1]

Compounds of the invention include deuterated versions of the disclosed compounds, for example, having one or more deuterium atoms in place of hydrogen.

In certain embodiments, the compound has one deuterium atom in place of a hydrogen atom.

In another aspect, the invention generally relates to a pharmaceutical composition comprising a compound disclosed herein and a pharmaceutically acceptable excipient, carrier, or diluent.

In certain embodiments, the pharmaceutical composition is suitable for oral administration.

In yet another aspect, the invention generally relates to a unit dosage form comprising a pharmaceutical composition disclosed herein.

In certain embodiments, is in the form of a tablet. In certain embodiments, is in the form of a capsule.

In yet another aspect, the invention generally relates to a method for treating or reducing cancer, or a related disease or disorder, comprising administering to a subject in need thereof a therapeutically effective amount of a compound disclosed herein.

In certain embodiments, the subject being treated is further administered one or more of chemotherapy, radiotherapy, targeted therapy, immunotherapy and hormonal therapy.

In certain embodiments, the method leads to one or more of: inhibiting cancer cell proliferation; inhibiting cancer cell invasiveness; inducing apoptosis of cancer cells; inhibiting cancer cell metastasis; and inhibiting angiogenesis.

In certain embodiments, the invention relates to a method of inhibiting cancer cell proliferation in a subject, comprising administering to the subject a compound of the invention, or a pharmaceutically acceptable salt thereof, in an amount effective to inhibit cell proliferation.

In certain embodiments, the invention relates to a method of inhibiting cancer cell invasiveness in a subject, comprising administering to the subject a compound of the invention, or a pharmaceutically acceptable salt thereof, in an amount effective to inhibit cell invasiveness.

In certain embodiments, the invention relates to a method of inducing apoptosis in cancer cells in a subject, comprising administering to the subject a compound of the invention, or a pharmaceutically acceptable salt thereof, in an amount effective to induce apoptosis.

In certain embodiments, the invention relates to a method of inhibiting cancer cell metastasis in a subject, comprising administering to the subject a compound of the invention, or a pharmaceutically acceptable salt thereof, in an amount effective to inhibit cell metastasis.

In certain embodiments, the invention relates to a method of inhibiting angiogenesis in a subject, comprising administering to the subject a compound of the invention, or a pharmaceutically acceptable salt thereof, in an amount effective to inhibit angiogenesis.

Examples of diseases or disorders that may be treated or reduced by compositions or methods of the invention include, but are not limited to, tumors, cancers, inflammatory diseases, autoimmune diseases, and the like.

In certain embodiments, the compound of Formula I is administered to treat one or more of breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer or thyroid cancer.

In certain embodiments, the cancer is breast cancer, ovarian cancer, bladder cancer, uterine cancer, prostate cancer, lung cancer, esophageal cancer, liver cancer, pancreatic cancer or stomach cancer.

In certain embodiments, the compound of Formula I is administered to treat breast cancer, including, e.g., ER-positive/HR-positive, HER2-negative breast cancer; ER-positive/HR-positive, HER2-positive breast cancer; triple negative breast cancer (TNBC); or inflammatory breast cancer.

In certain embodiments, the breast cancer is endocrine resistant breast cancer, trastuzumab resistant breast cancer, or breast cancer demonstrating primary or acquired resistance to CDK4/CDK6 inhibition.

In certain embodiments, the breast cancer is advanced or metastatic breast cancer.

In certain embodiments, the breast cancer is characterized by amplification or overexpression of CCNE1 and/or CCNE2.

In certain embodiments, the abnormal cell growth is cancer characterized by amplification or overexpression of CCNE1 and/or CCNE2.

In certain embodiments of the methods provided herein, the subject is identified as having a cancer characterized by amplification or overexpression of CCNE1 and/or CCNE2.

In certain embodiments, the cancer is selected from the group consisting of breast cancer and ovarian cancer.

In certain such embodiments, the cancer is breast cancer or ovarian cancer characterized by amplification or overexpression of CCNE1 and/or CCNE2.

In certain embodiments, the cancer is (a) breast cancer or ovarian cancer; (b) characterized by amplification or overexpression of cyclin E1 (CCNE1) or cyclin E2 (CCNE2); or (c) both (a) and (b). In some embodiments, the cancer is ovarian cancer.

In yet another aspect, the invention generally relates to use of a compound disclosed herein, and a pharmaceutically acceptable excipient, carrier, or diluent, in preparation of a medicament for treating a disease or disorder.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, PA (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary, shaping the product.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

The amount of the active compound administered will be dependent on the subject being treated, the severity of the disorder or condition, the route of administration, the disposition of the compound and the discretion of the prescribing physician. In some instances, dosage levels below the lower limit of the aforesaid range may be more than adequate, while in other cases still larger doses may be used without causing any harmful side effect, with such larger doses typically divided into several smaller doses for administration throughout the day.

Any appropriate route of administration can be employed, for example, oral, intramuscular, intravenous, transdermal, subcutaneous, sublingual, parenteral, nasal, pulmonary, inhalational, buccal, intraperitoneal, rectal, intrapleural, and intrathecal administration. Most suitable means of administration for a particular patient will depend on the nature and severity of the disease or condition being treated or the nature of the therapy being used and on the nature of the active compound.

In certain preferred embodiments, the compound is administered orally. Pharmaceutical compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued US Patents, some of which include, but are not limited to, U.S. Pat. Nos. 4,369,172; and 4,842,866, and references cited therein. Coatings can be used for delivery of compounds to the intestine (see, e.g., U.S. Pat. Nos. 6,638,534, 5,217,720, and 6,569,457, 6,461,631, 6,528,080, 6,800,663, and references cited therein). A useful formulation for the compounds of this invention is the form of enteric pellets of which the enteric layer comprises hydroxypropylmethylcellulose acetate succinate.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Methods of treatment disclosed herein may be employed in combination with or in addition to other therapies. In certain embodiments, the subject being treated is further administered one or more of chemotherapy, radiotherapy, targeted therapy, immunotherapy, and hormonal therapy.

Exemplary additional therapeutically active agents include, but are not limited to, small organic molecules such as drug compounds, e.g., compounds approved by the U.S. Food and Drug Administration (FDA) as provided in the Code of Federal Regulations (CFR), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, a compound of the invention may be administered in combination with endocrine therapy, e.g., agents such as letrozole, fulvestrant, tamoxifen, exemestane, or anastrozole.

In some embodiments, a compound of the invention may be administered in combination with a chemotherapeutic agent, e.g., docetaxel, paclitaxel, cisplatin, carboplatin, capecitabine, gemcitabine or vinorelbine. In other embodiments, a compound of the invention may be administered in combination with an anti-HER2 agent, e.g., trastuzumab or pertuzumab.

In certain embodiments, the method disclosed herein is in combination with one or more of immune check point blockade, co-signaling of T cells, and tumor targeting antibody therapies.

In certain embodiments, the method further comprises administering a chemotherapeutic agent to the subject.

In certain embodiments, the method further comprises administering a radiotherapy to the subject. In certain embodiments, the method further comprises administering a targeted therapy to the subject. In certain embodiments, the method further comprises administering an immunotherapy to the subject. In certain embodiments, the method further comprises administering hormonal therapy to the subject.

As used herein, the term “chemotherapeutic agent” refers to a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (Angew Chem. Intl. Ed. Engl. (1994) 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esonibicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP— 16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

Examples of the second (or further) agent or therapy may include, but are not limited to, immunotherapies (e.g. PD-1 inhibitors (pembrolizumab, nivolumab, cemiplimab), PD-L1 inhibitors (atezolizumab, avelumab, durvalumab), CTLA4 antagonist, cell signal transduction inhibitors (e.g., imatinib, gefitinib, bortezomib, erlotinib, sorafenib, sunitinib, dasatinib, vorinostat, lapatinib, temsirolimus, nilotinib, everolimus, pazopanib, trastuzumab, bevacizumab, cetuximab, ranibizumab, pegaptanib, panitumumab and the like), mitosis inhibitors (e.g., paclitaxel, vincristine, vinblastine and the like), alkylating agents (e.g., cisplatin, cyclophosphamide, chromabucil, carmustine and the like), anti-metabolites (e.g., methotrexate, 5-FU and the like), intercalating anticancer agents, (e.g., actinomycin, anthracycline, bleomycin, mitomycin-C and the like), topoisomerase inhibitors (e.g., irinotecan, topotecan, teniposide and the like), immunotherapy agents (e.g., interleukin, interferon and the like) and antihormonal agents (e.g., tamoxifen, raloxifene and the like).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (3H) and carbon-14 (¹⁴C) labeled compounds are particularly preferred for their ease of preparation and detectability. Further, substitution with heavier isotopes such as deuterium (2H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservatives, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are given for the purpose of illustrating the invention, but not for limiting the scope or spirit of the invention.

Compounds of the invention, including those specifically disclosed herein above and herein below, may be prepared as described in the following schemes. Although the present invention has been described in detail with preferred embodiments, those of ordinary skill in the art should understand that modifications, variations, and equivalent replacements made to the present invention within the scope of the present invention belong to the protection of the present invention.

### Abbreviations

| Abbreviation | Name |
|---|---|
| DCM | Dichloromethane |
| DMAP | 4-Dimethylaminopyridine |
| DMSO | Dimethyl sulfoxide |
| EDTA | Ethylenediaminetetraacetic acid |
| EA or tOAc | Ethyl acetate |
| HPLC | High-performance liquid chromatography |
| LCMS | Liquid chromatography-mass spectrometry |
| O/N | Overnight |
| PBS | Phosphate-buffered saline |
| Pd2(dba)3 | Tris(dibenzylideneacetone)dipalladium |
| PE | Petroleum ether |
| PMB | p-Methoxybenzyl |
| RT or rt | Room temperature |
| SFC | Supercritical fluid chromatography |
| TBS | tert-Butyldimethylsilyl |
| THF | Tetrahydrofuran |
| TLC | Thin-layer chromatography |
| Xantphos | (9,9-Dimethyl-9H-xanthene-4,5- |
| | diyl)bis(diphenylphosphane) |

| Name | Source |
|---|---|
| CDK1/cyclinB | BPS |
| CDK2/CycA2 | Carna |
| CDK2/CycE1 | Carna |
| Peptide18 | GL |
| EDTA | Gibco |
| HEPES, pH7.5 | Gibco |
| Brij-35 solution | Sigma |
| MgCl2 | Sigma |
| DTT | Sigma |
| EGTA | Sigma |
| 96 well plate | Corning |
| 384well plate | Corning |

### LC-MS Method:

Shimadzu LCMS2020, Reverse-phase column (Shim-Pack Scepter C18, 33×3.0 mm, 3 um), elution with A:H2O/MeCN/FA=90/10/0.05; B:MeCN; Detection: MS, ELS, UV (100 L split to MS with in-line UV detector); MS ionization method: Electrospray (positive and negative ion). ES-API=electrospray-atmospheric pressure ionization.

### HPLC Purification Method:

Instrument=Shimadzu FRC-40; Shimadzu LH-40; Shimadzu LC-8A; GX-281. Column=YMC-Triart C18, 250*20 mm, 5 um; Welch Ultimate XB-C18, 250*21.2 mm, 5 um. Detection wavelength=220, 254 nM. Flow rate=15 ml/min-20 ml/min; Run time=8 min; Column temperature=25° C.

### Compound Synthesis

### General Procedure 1

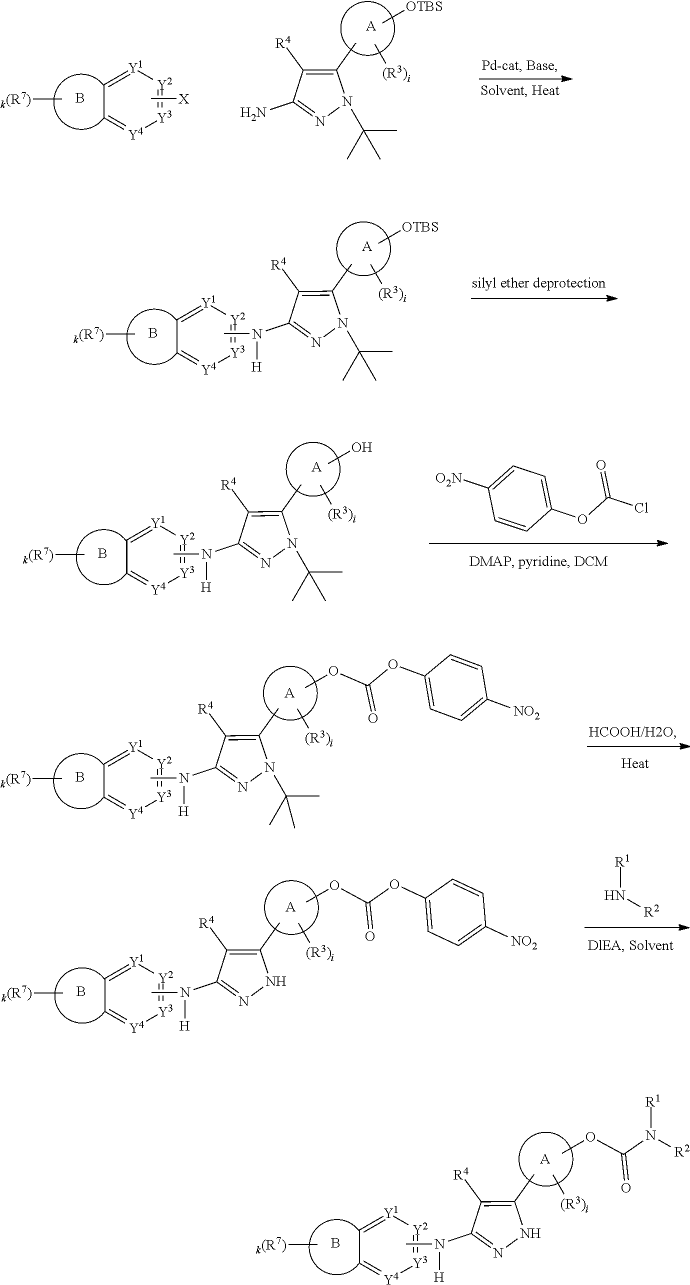

### General Procedure 2

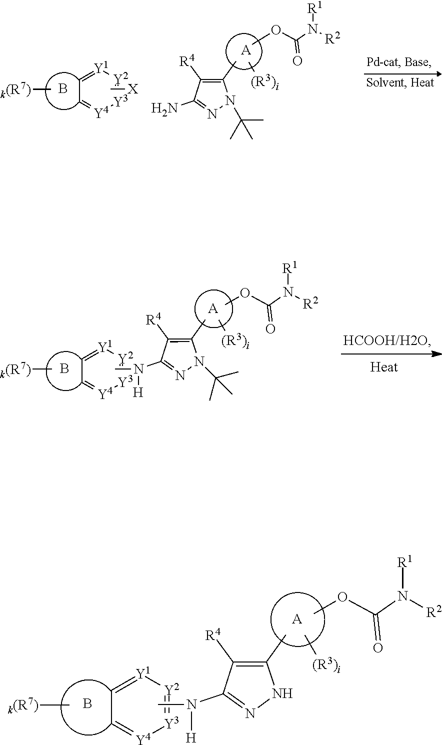

### Synthesis of Common Intermediate 1: 1-(Tert-Butyl)-3-((1S,3R)-3-((Tert-Butyldimethylsilyl)Oxy)Cyclopentyl)-1H-Pyrazol-5-Amine

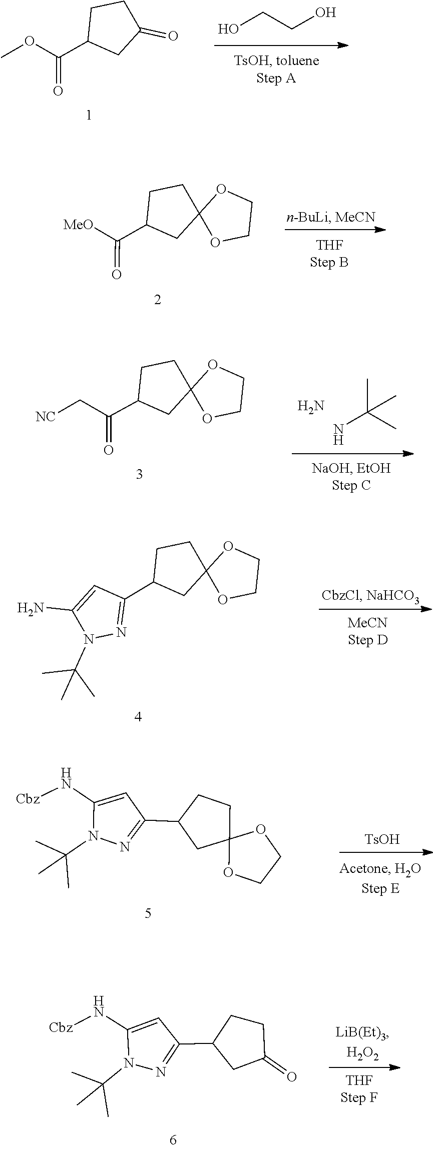

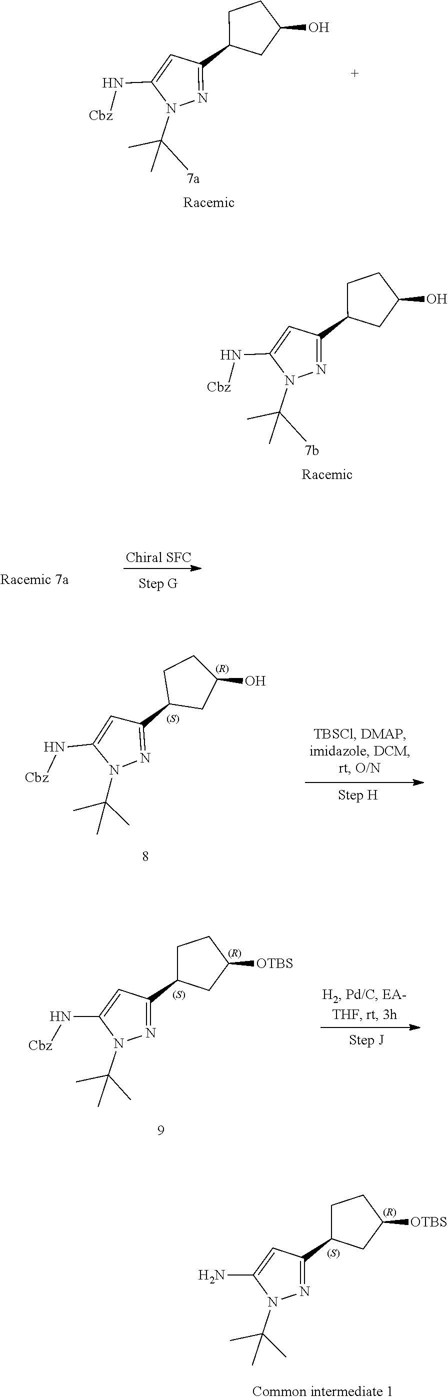

Step A. methyl 1,4-dioxaspiro[4.4]nonane-7-carboxylate. To a stirred solution of methyl 3-oxocyclopentane-1-carboxylate (70 g, 492.9 mmol) in toluene (600 mL) was added ethane-1,2-diol (61.1 g, 985.8 mmol), molecular sieve (15 g) and TsOH (8.5 g, 49.3 mmol) at rt. After stirring at 110° C. O/N, the cooled mixture was concentrated. The residue was purified by chromatography (silica gel, 0-20%, EtOAc in PE) to give methyl 1,4-dioxaspiro[4.4]nonane-7-carboxylate (34.8 g, 187.1 mmol, 31%) as a colorless oil.

Step B. 3-{1,4-dioxaspiro[4.4]nonan-7-yl}-3-oxopropanenitrile. To a stirred solution of n-BuLi (90 mL, 224.5 mmol, 2.5 M in hexane) in THF (200 mL) was added dropwise MeCN (15.3 g, 374.2 mmol) at −60° C. After stirring at −70° C. for 1 h. A solution of methyl 1,4-dioxaspiro[4.4]nonane-7-carboxylate (34.8 g, 187.1 mmol) in THF (50 mL) was added dropwise at −60° C. After stirring at −70° C. for 2 h, the reaction mixture was quenched with water (100 mL), adjust pH=7 with 1N aqueous HCl, extracted with ethyl acetate (50 mL*3). The combined organic layers were washed with brine (30 mL), dried over Na₂SO₄ and concentrated under reduced pressure to give crude 3-{1,4-dioxaspiro[4.4]nonan-7-yl}-3-oxopropanenitrile (32.8 g, 168.2 mmol, 90%) as a yellow oil which was used in the next step directly.

Step C. 1-tert-butyl-3-{1,4-dioxaspiro[4.4]nonan-7-yl}-1H-pyrazol-5-amine. To a stirred solution of sodium hydroxide (13.5 g, 336.4 mmol) in EtOH (300 mL) was added tert-butylhydrazine (31.3 g, 252.3 mmol) at rt. After stirring at rt for 1 h, 3-{1,4-dioxaspiro[4.4]nonan-7-yl}-3-oxopropanenitrile (32.8 g, 168.2 mmol) was added. After stirring at 80° C. O/N, the mixture was filtered. The filtrate was poured into water, adjusted PH to 6-7 with 1M aqueous HCl and extracted with EtOAc (80 mL×2). The combined organic phase was washed by water and brine, dried over Na₂SO₄, filtered and concentrated. The residue was purified by chromatography (silica gel, 0-50%, EtOAc in PE) to give 1-tert-butyl-3-{1,4-dioxaspiro[4.4]nonan-7-yl}-1H-pyrazol-5-amine (30 g, 113 mmol, 67%) as a yellow oil. LCMS: m/z 266 [M+H]⁺.

Step D. Benzyl N-(1-tert-butyl-3-{1,4-dioxaspiro[4.4]nonan-7-yl}-1H-pyrazol-5-yl)carbamate. To a stirred solution of 1-tert-butyl-3-{1,4-dioxaspiro[4.4]nonan-7-yl}-1H-pyrazol-5-amine (30 g, 113 mmol) in MeCN (350 mL) was added NaHCO₃ (47.5 g, 565.3 mmol) and Cbz-Cl (29 g, 169.6 mmol) at 0° C. After stirring at rt O/N, the mixture was filtered. The filtrate was concentrated and purified by chromatography (silica gel, 0-40%, EtOAc in PE) to give benzyl N-(1-tert-butyl-3-{1,4-dioxaspiro[4.4]nonan-7-yl}-1H-pyrazol-5-yl)carbamate (28 g, 70 mmol, 62%) as a yellow oil. LCMS: m/z 400 [M+H]⁺.

Step E. Benzyl N-[1-tert-butyl-3-(3-oxocyclopentyl)-1H-pyrazol-5-yl]carbamate. To a stirred solution of benzyl N-(1-tert-butyl-3-{1,4-dioxaspiro[4.4]nonan-7-yl}-1H-pyrazol-5-yl)carbamate (28 g, 70 mmol) in acetone (150 mL) was added H₂O (150 mL) and TsOH (6 g, 35 mmol) at rt. After stirred at 60° C. O/N, the cooled mixture was poured into water (150 ml) and extracted with EtOAc (50 ml×2). The combined organic phase was washed with brine, dried over Na₂SO₄, filtered and concentrated. The residue was purified by chromatography (silica gel, 0-50%, EtOAc in PE) to give benzyl N-[1-tert-butyl-3-(3-oxocyclopentyl)-1H-pyrazol-5-yl]carbamate (21 g, 59 mmol, 84%) as a white solid. LCMS: m/z 356 [M+H]⁺. 1H NMR (400 MHz, DMSO) δ 9.07 (s, 1H), 7.36-7.25 (m, 5H), 5.97 (s, 1H), 5.06 (s, 2H), 3.30-3.23 (m, 1H), 2.43-2.33 (m, 1H), 2.27-2.20 (m, 1H), 2.19-2.10 (m, 3H), 1.90-1.81 (m, 1H), 1.42 (s, 9H).

Step F. Benzyl N-[1-tert-butyl-3-(3-hydroxycyclopentyl)-1H-pyrazol-5-yl]carbamate. To a stirred solution of benzyl N-[1-tert-butyl-3-(3-oxocyclopentyl)-1H-pyrazol-5 -yl]carbamate (10 g, 28.134 mmol) in THF (100 mL) was added slowly LiBH(Et)₃ (56 mL, 56 mmol, 1 M in THF) at −65° C. After stirred at −65° C. for 1.5 h, NaHCO₃(aq, 40 mL) was added to the mixture at −30° C. (the mixture turned slowly into a glassy solid), then H₂O₂(19 g, 562 mmol) was slowly added to the mixture from −10° C. to rt (the glassy solid slowly backed into a normal mixture that can be easily stirred). After stirring at rt for further 1.5 h, the following mixture was poured slowly into saturated Na₂S₂O₃ (aq, 100 mL) and extracted with EtOAc (30 mL×2). The combined organic phase was washed with brine, dried over Na₂SO₄, filtered and concentrated. TLC (DCM: MeOH=10:1) showed a low polarity spot, which was the desired cis product. The residue was purified by chromatography (silica gel, 0-3%, MeOH in DCM) to give the cis isomer of benzyl N-[1-tert-butyl-3-(3-hydroxycyclopentyl)-1H-pyrazol-5-yl]carbamate (6.6 g, 18.5 mmol, 65%, less polar spot) as a white solid. LCMS: m/z 358 [M+H]+. 1H NMR (400 MHz, DMSO) δ 9.07 (s, 1H), 7.49-7.21 (m, 5H), 5.93 (s, 1H), 5.12 (s, 2H), 4.59 (d, J=4.4 Hz, 1H), 4.15 (dd, J=10.4, 4.5 Hz, 1H), 2.96-2.77 (m, 1H), 2.30-2.10 (m, 1H), 1.90-1.81 (m, 1H), 1.78-1.65 (m, 2H), 1.63-1.55 (m, 1H), 1.48 (s, 9H).

Step G. Benzyl N-{1-tert-butyl-3-[(1S,3R)-3-hydroxycyclopentyl]-1H-pyrazol-5-yl}carbamate. Benzyl N-[1-tert-butyl-3-(3-hydroxycyclopentyl)-1H-pyrazol-5-yl]carbamate (6.6 g, 18.5 mmol) was purified by prep-SFC to give benzyl N-{1-tert-butyl-3-[(1S,3R)-3-hydroxycyclopentyl]-1H-pyrazol-5-yl}carbamate (3.1 g, 8.6 mmol, 46.7%) as a white solid. LCMS: m/z 358 [M+H]⁺.

Step H. Benzyl (1-(tert-butyl)-3-((1S,3R)-3-((tert-butyldimethylsilyl)oxy)cyclopentyl)-1H-pyrazol-5-yl)carbamate. To a solution of benzyl N-{1-tert-butyl-3-[(1S,3R)-3-hydroxycyclopentyl]-1H-pyrazol-5-yl}carbamate (6.2 g, 17.3 mmol) in DCM (100 mL) were added DMAP (0.21 g, 1.73 mmol), 1H-Imidazol (2.36 g, 34.7 mmol), and TBSCl (2.61 g, 17.3 mmol) at rt. The reaction mixture was stirred at room temperature O/N. The reaction was concentrated in vacuo. The residue was purified using silica gel column chromatography eluting with EA in PE [gradient: 0-50%] to afford the title compound benzyl N-{1-tert-butyl-3-[(1S,3R)-3-[(tert-butyldimethylsilyl)oxy]cyclopentyl]-1H-pyrazol-5-yl}carbamate (7 g, 14.8 mmol, 85.7%) as a colorless oil. LCMS: m/z 472 [M+H]⁺.

Step I. 1-(tert-butyl)-3-((1S,3R)-3-((tert-butyldimethylsilyl)oxy)cyclopentyl)-1H-pyrazol-5-amine. To a solution of benzyl N-{1-tert-butyl-3-[(1S,3R)-3-[(tert-butyldimethylsilyl)oxy]cyclopentyl]-1H-pyrazol-5-yl}carbamate (7 g, 14.8 mmol) in THF (50 mL) and ethyl acetate (50 mL) was added Pd/C 10% (0.20 g, 1.86 mmol) at rt. The reaction mixture was stirred at rt for 3 h under H₂ using a H₂ balloon. The reaction mixture was filtered and concentrated to afford the title compound 1-tert-butyl-3-[(1S,3R)-3-[(tert-butyldimethylsilyl)oxy]cyclopentyl]-1H-pyrazol-5-amine (4.8 g, 14.2 mmol, 96%) as a colorless oil. LCMS: m/z 338 [M+H]⁺. ¹H NMR (400 MHz, CDCl3) δ 5.47 (s, 1H), 4.32-4.08 (m, 1H), 3.47 (s, 2H), 2.98-2.80 (m, 1H), 2.34-2.19 (m, 1H), 1.98-1.89 (m, 1H), 1.85-1.75 (m, 2H), 1.72-1.64 (m, 1H), 1.63-1.59 (m, 10H), 0.90-0.88 (m, 9H), 0.05 (d, J=2 Hz, 6H).

### Synthesis Common Intermediate 2: (1R,3S)-3-[5-Amino-1-(2-Methylprop-2-yl)Pyrazol-3-yl]Cyclopentyl (Prop-2-ylamino)Methanoate

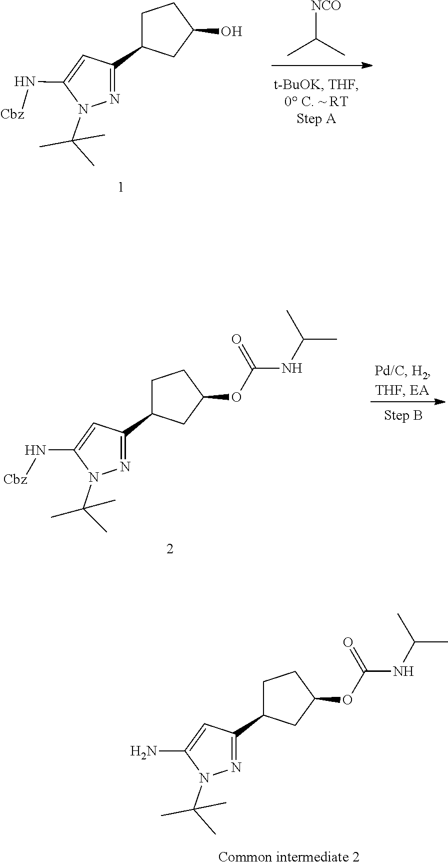

Step A: bezl {[2-(2-methylprop-2-yl)-5-[(1s,3R)-3-{[prop-2-ylamino)carbonyl]oxy}cyclopentyl]pyrazol-3-yl]amino}methanoate To a solution of benzyl ({5-[(1S,3R)-3-hydroxycyclopentyl]-2-(2-methylprop-2-yl)pyrazol-3-yl}amino)methanoate (1.0 g, 2.8 mmol) in THF (20 mL) were added t-BuOK (4.2 mL, 1M in THF) at 0° C. and the reaction was stirred at 0° C. for 30 min. A solution of 2-Isocyanatopropane (290 mg, 3.6 mmol) in 5 mL THF was added dropwise into the reaction at 0° C. The reaction mixture was allowed to warm to room temperature and stirred for 1 h, TLC and LCMS indicated the reaction was over. The reaction was poured into ice-water and extracted with EA (30 mL*3). The combined organic layer was separated, washed with brine, dried over Na₂SO4, filtered and concentrated. The residue was purified by silica gel column chromatography eluted with methanol in dichloromethane. (Gradient: 0-3%) to afford the title compound benzyl {[2-(2-methylprop-2-yl)-5-[(1S,3R)-3-{[(prop-2-ylamino)carbonyl]oxy}cyclopentyl]pyrazol-3-yl]amino}methanoate (1.2 g, 2.7 mmol, 96.9%) as a white solid. LCMS: m/z 443 [M+H]⁺. ¹H NMR (400 MHz, DMSO) δ 9.07 (s, 1H), 7.45-7.33 (m, 5H), 6.92 (d, J=7.6 Hz, 1H), 5.93 (s, 1H), 5.11 (s, 2H), 4.97 (s, 1H), 3.60-3.50 (m, 1H), 3.01-2.88 (m, 1H), 2.40-2.29 (m, 1H), 1.98-1.77 (m, 2H), 1.75-1.56 (m, 3H), 1.47 (s, 9H), 1.02 (d, J=6.4 Hz, 6H).

Step B: (1R,3S)-3-[5-amino-1-(2-methylprop-2-yl)pyrazol-3-yl]cyclopentyl (prop-2-ylamino)methanoate A suspension of benzyl {[2-(2-methylprop-2-yl)-5-[(1S,3R)-3-{[(prop-2-ylamino)carbonyl]oxy}cyclopentyl]pyrazol-3-yl]amino}methanoate (1.2 g, 2.7 mmol) and Pd/C 10% (290 mg) in THF (10 mL) and EA (10 mL) was stirred at room temperature under H₂ atmosphere using a balloon for 18 h. The reaction mixture was filtered and concentrated. The residue was purified by silica gel column chromatography eluting with methanol in dichloroform (gradient: 0-6%) to afford the title compound (1R,3S)-3-[5-amino-1-(2-methylprop-2-yl)pyrazol-3-yl]cyclopentyl (prop-2-ylamino)methanoate (700 mg, 2.2 mmol, 83.7%) as syrupy solid. LCMS: m/z 309 [M+H]⁺. ¹H NMR (400 MHz, DMSO) δ 6.88 (d, J=7.6 Hz, 1H), 5.22 (s, 1H), 4.94 (s, 1H), 4.70 (s, 2H), 3.62-3.53 (m, 1H), 2.84-2.75 (m, 1H), 2.34-2.24 (m, 1H), 1.86-1.76 (m, 2H), 1.68-1.53 (m, 3H), 1.48 (s, 9H), 1.03 (d, J=6.4 Hz, 6H).

### Synthesis Common Intermediate 3: 1-(tert-butyl)-3-((1s,4s)-4-((tert-butyldiphenylsilyl)oxy)cyclohexyl)-1H-pyrazol-5-amine

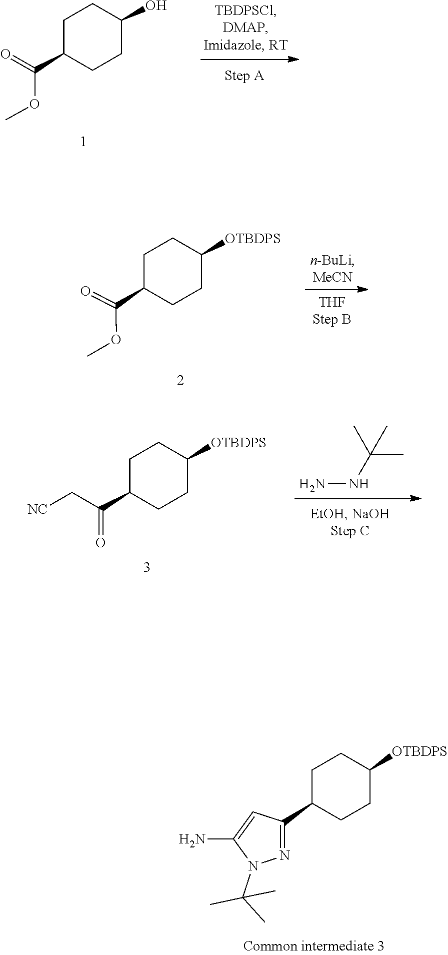

Step A: methyl (1s,4s)-4-((tert-butyldiphenylsilyl)oxy)cyclohexane-1-carboxylate To a solution of methyl (1s,4s)-4-hydroxycyclohexane-1-carboxylate (5 g, 31.6 mmol) in DCM (30 mL) were added DMAP (0.39 g, 3.16 mmol), Imidazole (6.46 g, 94.8 mmol), and TBDPSCl (9.83 mL, 37.9 mmol), and the reaction was stirred at room temperature for overnight. The reaction was concentrated in vacuo. The residue was purified using silica gel column chromatography eluting with EA in PE [gradient: 0-50%] to afford the title compound methyl (1s,4s)-4-[(tert-butyldiphenylsilyl)oxy]cyclohexane-1-carboxylate (10.1 g, 25.4 mmol, 80.4%) as a colorless oil. LCMS: ESI m/z 397 [M+H]⁺

Step B: 3-((1s,4s)-4-((tert-butyldiphenylsilyl)oxy)cyclohexyl)-3-oxopropanenitrile A solution of n-BuLi (20.0 mL, 50.0 mmol) in THF (60 mL) was cooled to −65° C. ACN (1.48 g, 31.5 mmol) was added dropwise at −65° C. The reaction mixture was stirred at −65° C. for 1 hr. A solution of methyl (1s,4s)-4-((tert-butyldiphenylsilyl)oxy)cyclohexane-1-carboxylate (5.00 g, 12.6 mmol) in THF (10 mL) was added dropwise at −65° C. The resulting mixture was stirred at −65° C. for 1 hr. After TLC showed completion, the reaction mixture was quenched with water (50 mL), adjust pH=7 with 1N HCl, extracted with EA (50 mL*3). The combined organic layers were washed with brine (30 mL), dried over Na₂SO₄ and concentrated under reduced pressure to give 3-((1s,4s)-4-((tert-butyldiphenylsilyl)oxy)cyclohexyl)-3-oxopropanenitrile (5.10 g, 12.6 mmol, 99.8%) as an oil which was used for next step directly.

Step C: 1-(tert-butyl)-3-((1s,4s)-4-((tert-butyldiphenylsilyl)oxy)cyclohexyl)-1H-pyrazol-5-amine To a solution of N-tert-butylhydroxylamine (1.91 g, 21.4 mmol) in EtOH (50 mL) was added NaOH (0.6 g, 15.1 mmol). The reaction mixture was stirred at room temperature for 1 hr, then 3-((1s,4s)-4-((tert-butyldiphenylsilyl)oxy)cyclohexyl)-3-oxopropanenitrile (5.10 g, 12.6 mmol) was added. The following mixture was stirred at 80° C. overnight. LCMS showed the reaction was complete. The cooled reaction mixture was filtered and concentrated. The residue was purified via a FC on silica gel eluting with PE/EA 1:1 as a yellow solid 1-(tert-butyl)-3-((1s,4s)-4-((tert-butyldiphenylsilyl)oxy)cyclohexyl)-1H-pyrazol-5-amine (4.20 g, 8.83 mmol, 70%). LC-MS: ESI m/z 476.75 [M+H]⁺. ¹H NMR (400 MHz, DMSO) δ 7.65-7.57 (m, 4H), 7.48-7.39 (m, 6H), 5.25 (s, 1H), 4.71 (s, 2H), 4.00 (brs, 1H), 2.40-2.31 (m, 1H), 1.89-1.78 (m, 2H), 1.67-1.59 (m, 2H), 1.58-1.49 (m, 2H), 1.49 (s, 9H), 1.45-1.37 (m, 2H), 1.04 (s, 9H).

### Synthesis Common Intermediate 4: (1s,3s)-3-(5-amino-1-(tert-butyl)-1H-pyrazol-3-yl)cyclobutyl isopropylcarbamate

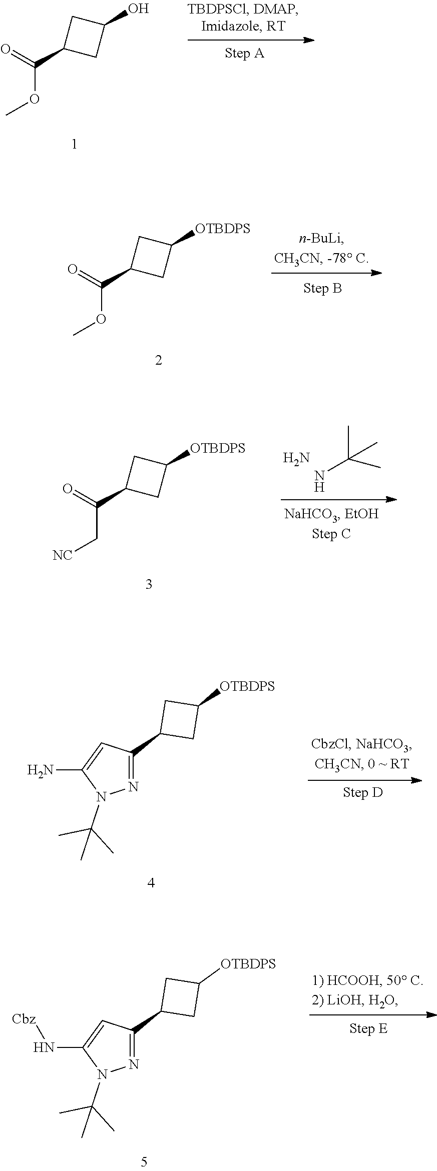

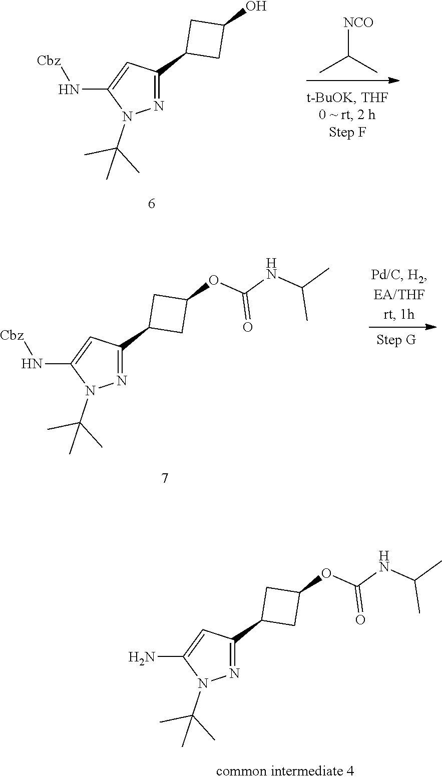

Step A: methyl (1s,3s)-3-((tert-butyldiphenylsilyl)oxy)cyclobutane-1-carboxylate To a solution of methyl (1s,3s)-3-hydroxycyclobutane-1-carboxylate (5 g, 31.6 mmol) in DCM (30 mL) were added DMAP (0.39 g, 3.16 mmol), Imidazole (6.46 g, 94.8 mmol), and TBDPSCl (9.83 mL, 37.9 mmol), and the reaction was stirred at room temperature overnight. The reaction was concentrated in vacuo. The residue was purified using silica gel column chromatography eluting with EA in PE [gradient: 0-50%] to afford the title compound methyl (1s,3s)-3-((tert-butyldiphenylsilyl)oxy)cyclobutane-1-carboxylate (14 g, 25.4 mmol, 80.4%) as a colorless oil. LCMS: ESI m/z 369.35 [M+H]⁺

Step B: 3-((1s,3s)-3-((tert-butyldiphenylsilyl)oxy)cyclobutyl)-3-oxopropanenitrile To a stirred slurry of n-BuLi (10.0 mL, 25.2 mmol) in dry THF (150 mL) containing CH₃CN (1.98 mL, 37.8 mmol), at −45° C. under N2, was added methyl methyl (1s,3s)-3-((tert-butyldiphenylsilyl)oxy)cyclobutane-1-carboxylate (5 g, 12.6 mmol) The reaction mixture was stirred at −45° C. for 2 h. The reaction was diluted with H₂O. The aqueous layer was extracted with EA. Combined the EA layer and washed with saturated NaCl solution. The EA layer was dried by Na₂SO₄, filtered and concentrated in vacuo. The compound 3-((1s,3s)-3-((tert-butyldiphenylsilyl)oxy)cyclobutyl)-3-oxopropanenitrile (5 g, 8.62 mmol, 68%) was obtained as a yellow solid and used into next step without further purification. LCMS: ESI m/z 378.20 [M+H]+

Step C: 1-(tert-butyl)-3-((1s,3s)-3-((tert-butyldiphenylsilyl)oxy)cyclobutyl)-1H-pyrazol-5-amine To a solution of 3-((1s,3s)-3-((tert-butyldiphenylsilyl)oxy)cyclobutyl)-3-oxopropanenitrile (5 g, 12.3 mmol) in EtOH (100 mL) was added tert-butylhydrazine (2.17 g, 24.6 mmol) and NaHCO₃(2.07 g, 24.6 mmol), the reaction mixture was stirred at 80° C. for 18 hours. The residue was purified using silica gel column chromatography eluting with EA in PE [gradient: 0-50%] to afford 1-(tert-butyl)-3-((1s,3s)-3-((tert-butyldiphenylsilyl)oxy)cyclobutyl)-1H-pyrazol-5-amine (2.1 g, 3.73 mmol, 30%) as white solid. LCMS: ESI m/z 448.3 [M+H]

Step D: benzyl (1-(tert-butyl)-3-((1s,3s)-3-((tert-butyldiphenylsilyl)oxy)cyclobutyl)-1H-pyrazol-5-yl)carbamate To a solution of 1-(tert-butyl)-3-((1s,3s)-3-((tert-butyldiphenylsilyl)oxy)cyclobutyl)-1H-pyrazol-5-amine (1.50 g, 3.35 mmol) in ACN (15 mL) was added CbzCl (0.57 g, 3.35 mmol) and NaHCO₃(0.28 g, 3.35 mmol) at 0° C. for 30 min. The mixture was stirred at room temperature overnight. TLC (PE/EA=5:1) showed starting material was consumed and new spot was observed. The reaction mixture was diluted with water 50 mL, extracted with EA (50 mL×3). The organic phase was washed with brine, dried over Na₂SO₄ and concentrated. The residue was purified by silica gel chromatography (15 g column) using 0-20% EtOAc/hexane to afford benzyl (1-(tert-butyl)-3-((1s,3s)-3-((tert-butyldiphenylsilyl)oxy)cyclobutyl)-1H-pyrazol-5-yl)carbamate (1.2 g, 2.06 mmol, 61%) as a yellow oil. LCMS: 582 [M+H]⁺.

Step E: benzyl (1-(tert-butyl)-3-((1s,3s)-3-hydroxycyclobutyl)-1H-pyrazol-5-yl)carbamate A solution of benzyl (1-(tert-butyl)-3-((1s,3s)-3-((tert-butyldiphenylsilyl)oxy)cyclobutyl)-1H-pyrazol-5-yl)carbamate (1.2 g, 2.06 mmol) in HCOOH (10 mL) was stirred at 50° C. for 5 hr. Spin the cooled reaction liquid dry. The residue was dissolved in MeOH (5 mL) and H₂O (5 mL), LiOH (0.4 g, 10.3 mmol) was added. The following mixture was stirred at room temperature for 1 h. TLC (PE/EA=1:1) showed starting material was consumed and new spot was observed. The reaction mixture was diluted with water 50 mL, extracted with EA (50 mL×3). The organic phase was washed with brine, dried over Na₂SO₄ and concentrated. The residue was purified by silica gel chromatography (15 g column) using 0-50% EtOAc/hexane to afford benzyl (1-(tert-butyl)-3-((1s,3s)-3-hydroxycyclobutyl)-1H-pyrazol-5-yl)carbamate (670 mg, 1.95 mmol, 95%) as a colorless oil. LCMS: 344 [M+H]⁺. ¹H NMR (400 MHz, DMSO) δ 9.12 (s, 2H), 7.46-7.28 (m, 5H), 5.96 (s, 1H), 5.12 (s, 2H), 5.02 (d, J=6.8 Hz, 1H), 4.01-3.92 (m, 1H), 3.17 (d, J=5.2 Hz, 1H), 2.81-2.60 (m, 1H), 2.51-2.42 (m, 1H), 1.95-1.80 (m, 2H), 1.47 (s, 9H).

Step F: (1s,3s)-3-(5-(((benzyloxy)carbonyl)amino)-1-(tert-butyl)-1H-pyrazol-3-yl)cyclobutyl isopropylcarbamate To a solution of benzyl (1-(tert-butyl)-3-((1s,3s)-3-hydroxycyclobutyl)-1H-pyrazol-5-yl)carbamate (670 mg, 1.95 mmol) in THF (10 mL) was added potassium 2-methylpropan-2-olate (328 mg, 2.92 mmol) and 1-oxo-N-(prop-2-yl)methanimine (249 mg, 2.92 mmol) at 0° C. for 30 min. The reaction mixture was stirred at room temperature for 1 h. TLC (PE/EA=1:1) showed starting material was consumed and new spot was observed. The reaction mixture was diluted with water 50 mL, extracted with EA (50 mL×3). The organic phase was washed with brine, dried over Na₂SO₄ and concentrated. The residue was purified by silica gel chromatography (15.0 g column) using 0-50% EtOAc/hexane to afford (1s,3s)-3-(5-(((benzyloxy)carbonyl)amino)-1-(tert-butyl)-1H-pyrazol-3-yl)cyclobutyl isopropylcarbamate (530 mg, 1.24 mmol, 63.4%) as a white solid. LCMS: 429 [M+H]⁺. ¹H NMR (400 MHz, DMSO) δ 9.11 (s, 1H), 7.47-7.28 (m, 5H), 7.04 (d, J=7.2 Hz, 1H), 5.99 (s, 1H), 5.13 (s, 2H), 4.81-4.72 (m, 1H), 3.61-3.53 (m, 1H), 2.99-2.91 (m, 1H), 2.67-2.53 (m, 2H), 2.13-1.93 (m, 2H), 1.49 (d, J=4.4 Hz, 9H), 1.04 (d, J=6.4 Hz, 6H).

Step G: (1s,3s)-3-(5-amino-1-(tert-butyl)-1H-pyrazol-3-yl)cyclobutyl isopropylcarbamate To a solution of (1s,3s)-3-(5-(((benzyloxy)carbonyl)amino)-1-(tert-butyl)-1H-pyrazol-3-yl)cyclobutyl isopropylcarbamate (530 mg, 1.23 mmol) in THF (2.5 mL) and EA (2.5 mL) was added Pd/C (100 mg, 0.940 mmol) under H₂ protect. The mixture was stirred at room temperature for 1 h. TLC (PE/EA=1:1) showed starting material was consumed and new spot was observed. The reaction mixture was diluted with water 50 mL, extracted with EA (50 mL×3). The organic phase was washed with brine, dried over Na₂SO₄ and concentrated to afford (1s,3s)-3-(5-amino-1-(tert-butyl)-1H-pyrazol-3-yl)cyclobutyl isopropylcarbamate (350 mg, 1.19 mmol, 96%) as a colorless oil. LCMS: 295 [M+H]⁺. ¹H NMR (400 MHz, CDCl₃) δ 5.50 (s, 1H), 4.97-4.84 (m, 1H), 4.49 (s, 1H), 3.84-3.76 (m, 1H), 3.48 (s, 1H), 3.02-2.87 (m, 1H), 2.79-2.62 (m, 2H), 2.18-2.03 (m, 2H), 1.61 (s, 9H), 1.15 (d, J=6.4 Hz, 6H).

### Example 1: (1R,3S)-3-(3-((1-methyl-3-oxo-2,3-dihydro-1H-indazol-6-yl)amino)-1H-pyrazol-5-yl)cyclopentyl isopropylcarbamate

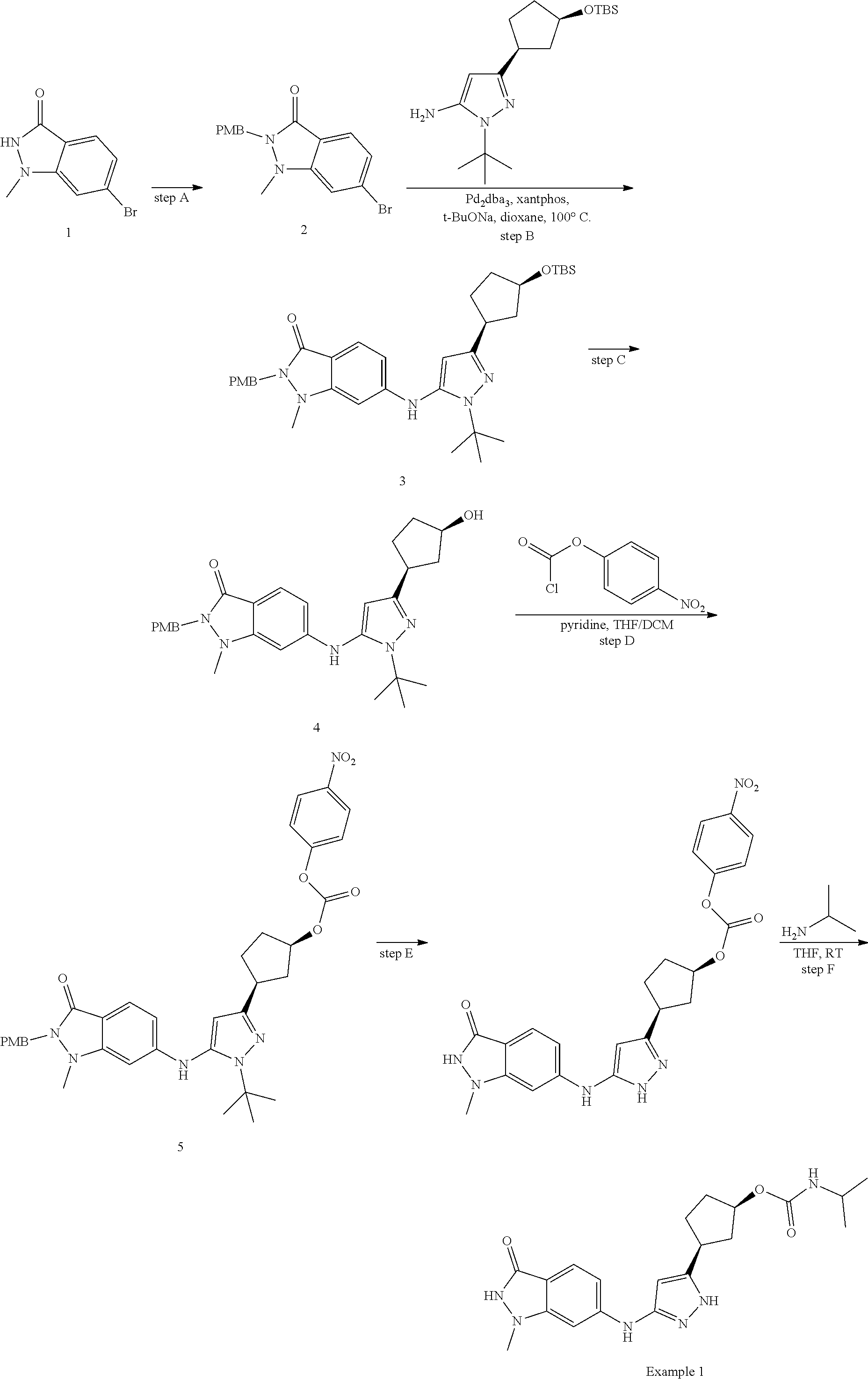

Step A: 6-bromo-2-(4-methoxybenzyl)-1-methyl-1,2-dihydro-3H-indazol-3-one To a solution of 6-bromo-1-methyl-2,3-dihydro-1H-indazol-3-one (0.5 g, 2.20 mmol) in DMF (10 mL) were added NaH (0.26 g, 6.60 mmol, 60% in mineral oil), and the reaction was stirred at room temperature for 30 min. After that 1-(chloromethyl)-4-methoxybenzene (0.360 mL, 2.642 mmol) was added. The reaction was stirred at room temperature for extra 2 hours. The reaction was diluted with EA and saturated NH₄Cl solution. The organic layer was separated, washed with further brine, and concentrated in vacuo. The residue was purified using silica gel column chromatography eluting with ethyl acetate in petroleum ether (½). The organic layer was collected, concentrated in vacuo, and dried to afford the title compound 6-bromo-2-[(4-methoxyphenyl)methyl]-1-methyl-2,3-dihydro-1H-indazol-3-one (300 mg, 0.864 mmol, 39.2%) as a yellow solid. LCMS: ESI m/z 347, 349 [M+H]⁺

Step B: 6-((1-(tert-butyl)-3-((1S,3R)-3-((tert-butyldimethylsilyl)oxy)cyclopentyl)-1H-pyrazol-5-yl)amino)-2-(4-methoxybenzyl)-1-methyl-1,2-dihydro-3H-indazol-3-one To a solution of 1-tert-butyl-3-[(1S,3R)-3-[(tert-butyldimethylsilyl)oxy]cyclopentyl]-1H-pyrazol-5-amine (320.85 mg, 0.950 mmol) in dioxane (5 mL) were added 6-bromo-2-[(4-methoxyphenyl)methyl]-1-methyl-2,3-dihydro-1H-indazol-3-one (300 mg, 0.864 mmol), [5-(diphenylphosphanyl)-9,9-dimethyl-9H-xanthen-4-yl]diphenylphosphane (99.99 mg, 0.173 mmol), sodium 2-methylpropan-2-olate (1.72 mL, 1.72 mmol) and Pd₂(dba)₃ (79.1 mg, 0.086 mmol), and the reaction was stirred at 90° C. under N₂ for 18 hr. The reaction was diluted with EA and water. The organic layer was separated, washed with further brine, and concentrated in vacuo. The residue was purified using silica gel column chromatography eluting with ethyl acetate in petroleum ether (3/1) The organic layer was collected, concentrated in vacuo, and dried to afford the title compound 6-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-1-methyl-2,3-dihydro-1H-indazol-3-one (200 mg, 0.331 mmol, 38.3%) as a yellow oil. LCMS: ESI m/z 604 [M+H]⁺

Step C: 6-((1-(tert-butyl)-3-((1S,3R)-3-hydroxycyclopentyl)-1H-pyrazol-5-yl)amino)-2-(4-methoxybenzyl)-1-methyl-1,2-dihydro-3H-indazol-3-one To a flask containing 6-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-1-methyl-2,3-dihydro-1H-indazol-3-one (200 mg, 0.331 mmol) was added formic acid (10 mL) The mixture was stirred at rt for 18 hr. The mixture was concentrated in vacuo to give a residue, which was purified by using silica gel column chromatography eluting with (DCM/MeOH=3:1) to afford the title compound 6-({1-tert-butyl-3-[(1S,3R)-3-hydroxycyclopentyl]-1H-pyrazol-5-yl}amino)-2-[(4-methoxyphenyl)methyl]-1-methyl-2,3-dihydro-1H-indazol-3-one (100 mg, 0.204 mmol, 61.7%) as a yellow oil. LC/MS (ESI) (m/z): 490 [M+H]⁺.

Step D: (1R,3S)-3-(1-(tert-butyl)-5-((2-(4-methoxybenzyl)-1-methyl-3-oxo-2,3-dihydro-1H-indazol-6-yl)amino)-1H-pyrazol-3-yl)cyclopentyl (4-nitrophenyl) carbonate To a flask containing 6-({1-tert-butyl-3-[(1S,3R)-3-hydroxycyclopentyl]-1H-pyrazol-5-yl}amino)-2-[(4-methoxyphenyl)methyl]-1-methyl-2,3-dihydro-1H-indazol-3-one (100 mg, 0.204 mmol) was added THF (10 mL) and DCM (10 mL) followed by the addition of 4-nitrophenyl chloroformate (82.33 mg, 0.408 mmol) and Pyridine (0.050 mL, 0.613 mmol). The mixture was stirred at rt for 2 hr. The mixture was concentrated in vacuo to give a residue, which was purified using silica gel column chromatography eluting with (PE/EA=1:3) to afford the title compound (1R,3S)-3-[1-tert-butyl-5-({2-[(4-methoxyphenyl)methyl]-1-methyl-3-oxo-2,3-dihydro-1H-indazol-6-yl}amino)-1H-pyrazol-3-yl]cyclopentyl 4-nitrophenyl carbonate (100 mg, 0.153 mmol, 74.8%) as a yellow oil. LCMS: ESI m/z 655 [M+H]⁺

Step E: (1R,3S)-3-(5-((1-methyl-3-oxo-2,3-dihydro-1H-indazol-6-yl)amino)-1H-pyrazol-3-yl)cyclopentyl (4-nitrophenyl) carbonate To a flask containing (1R,3S)-3-[1-tert-butyl-5-({2-[(4-methoxyphenyl)methyl]-1-methyl-3-oxo-2,3-dihydro-1H-indazol-6-yl}amino)-1H-pyrazol-3-yl]cyclopentyl 4-nitrophenyl carbonate (100 mg, 0.153 mmol) was added formic acid (5 mL). The mixture was stirred at 80° C. for 18 hr. After completion the solvent was removed on vacuum to give (1R,3S)-3-{5-[(1-methyl-3-oxo-2,3-dihydro-1H-indazol-6-yl)amino]-1H-pyrazol-3-yl}cyclopentyl 4-nitrophenyl carbonate (70 mg, 0.146 mmol, 96%) as a yellow oil, which was used into next step without further purification. LCMS: ESI m/z 479 [M+H]+

Step F: (1R,3S)-3-(3-((1-methyl-3-oxo-2,3-dihydro-1H-indazol-6-yl)amino)-1H-pyrazol-5-yl)cyclopentyl isopropylcarbamate To a solution of (1R,3S)-3-{5-[(1-methyl-3-oxo-2,3-dihydro-1H-indazol-6-yl)amino]-1H-pyrazol-3-yl}cyclopentyl 4-nitrophenyl carbonate (70 mg, 0.146 mmol) in propan-2-amine (5 mL), the reaction mixture was stirred at rt for 2 hours. The solvent was removed on vacuum to give a residue, which was purified by prep-HPLC (C18, FA condition, 56% ACN) to afford (1R,3S)-3-{5-[(1-methyl-3-oxo-2,3-dihydro-1H-indazol-6-yl)amino]-1H-pyrazol-3-yl}cyclopentyl N-(propan-2-yl)carbamate (3.7 mg) a white solid. LCMS: ESI m/z 399 [M+H]⁺. ¹H NMR (400 MHz, DMSO-d₆) δ 8.59 (s, 1H), 7.48 (s, 1H), 7.34 (d, J=8.4 Hz, 1H), 6.95 (d, J=7.6 Hz, 1H), 6.73 (d, J=8.4 Hz, 1H), 5.66 (s, 1H), 4.99 (s, 1H), 3.62-3.50 (m, 1H), 3.47 (s, 3H), 3.13-2.99 (m, 1H), 2.55-2.42 (m, 1H), 2.10-1.83 (m, 2H), 1.79-1.52 (m, 3H), 1.03 (d, J=6.4 Hz, 6H).

### Example 2: (1R,3S)-3-(3-((1,1-dioxido-3,4-dihydro-2H-benzo[e][1,2]thiazin-6-yl)amino)-1H-pyrazol-5-yl)cyclopentyl isopropylcarbamate

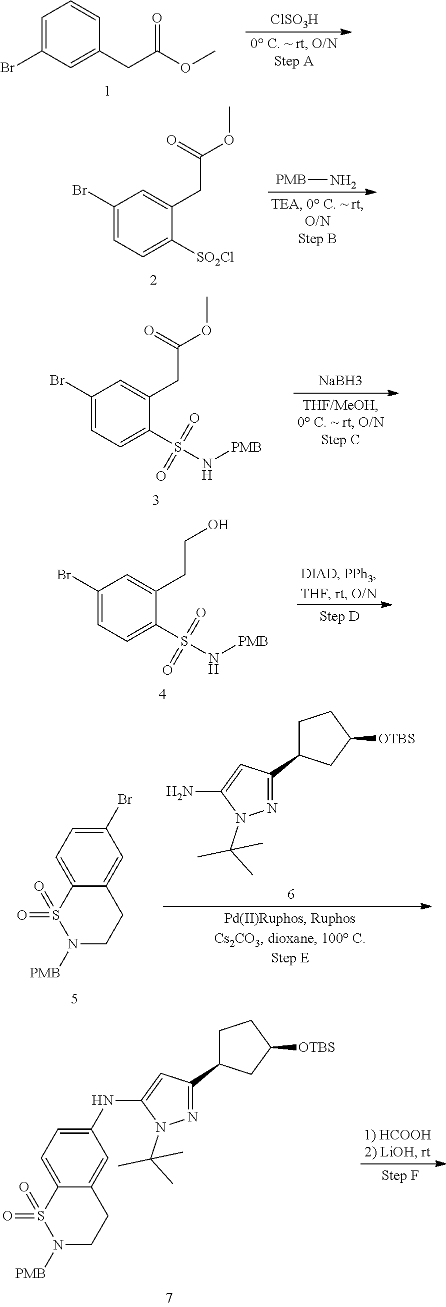

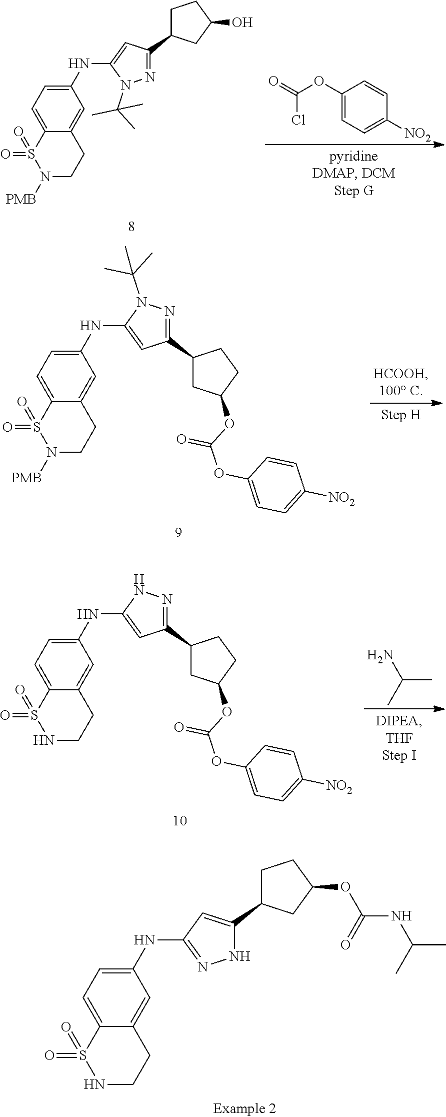

Step A. methyl 2-(5-bromo-2-(chlorosulfonyl)phenyl)acetate A mixture of methyl 2-(3-bromophenyl)acetate (3 g, 13.1 mmol) in HSO₃Cl (1.31 g, 13.1 mmol) was stirred at rt overnight. The mixture was poured into ice-water (50 ml) slowly and extracted with EtOAc (20 mL*3). The combined organic phase was washed with brine, dried over Na₂SO₄, filtered and concentrated to give crude methyl 2-[5-bromo-2-(chlorosulfonyl)phenyl]acetate (1.8 g, 5.52 mmol, 42.2%) as a yellow oil. LCMS: m/z 327 [M+H]⁺.

Step B. methyl 2-(5-bromo-2-(N-(4-methoxybenzyl)sulfamoyl)phenyl)acetate To a stirred mixture of methyl 2-[5-bromo-2-(chlorosulfonyl)phenyl]acetate (1.8 g, 5.49 mmol) in DCM (15 mL) was added (4-methoxyphenyl)methanamine (0.75 g, 5.49 mmol) and TEA (1.11 g, 10.9 mmol) slowly at 0° C. After stirred at rt for 2 h, the mixture was poured into water (30 mL) and extracted with DCM (20 mL*2). The combined organic phase was washed with brine, dried with Na₂SO₄, filtered and concentrated. The residue was purified by chromatography (silica gel, 0-35%, EtOAc in PE) to give methyl 2-(5-bromo-2-{[(4-methoxyphenyl)methyl]sulfamoyl}phenyl)acetate (800 mg, 1.86 mmol, 33.9%) as a brown solid. LCMS: m/z 429 [M+H]⁺.

Step C. 4-bromo-2-(2-hydroxyethyl)-N-(4-methoxybenzyl)benzenesulfonamide To a stirred mixture of methyl 2-(5-bromo-2-{[(4-methoxyphenyl)methyl]sulfamoyl}phenyl)acetate (800 mg, 1.86 mmol) in THF (10 mL)/MeOH (2 mL) was added NaBH₄ (189 mg, 5.60 mmol) slowly at 0° C. After stirred at rt for 1 h, the mixture was poured into ice-water (30 mL) and extracted with EtOAc (20 mL*2). The combined organic phase was washed with brine, dried with Na₂SO₄, filtered and concentrated to give crude 4-bromo-2-(2-hydroxyethyl)-N-[(4-methoxyphenyl)methyl]benzene-1-sulfonamide (700 mg, 1.74 mmol, 93.6%) as a yellow solid. LCMS: m/z 401 [M+H]⁺.

Step D. 6-bromo-2-(4-methoxybenzyl)-3,4-dihydro-2H-benzo[e][1,2]thiazine 1,1-dioxide To a stirred mixture of 4-bromo-2-(2-hydroxyethyl)-N-[(4-methoxyphenyl)methyl]benzene-1-sulfonamide (700 mg, 1.74 mmol) in THF (20 mL) was added DIAD (0.693 mL, 3.49 mmol) and PPh₃ (917 mg, 3.49 mmol) at rt. After stirred at rt overnight, the mixture was poured into water (30 mL) and extracted with EtOAc (20 mL*2). The combined organic phase was washed with brine, dried with Na₂SO₄, filtered and concentrated. The residue was purified by chromatography (silica gel, 0-50%, EtOAc in PE) to give 6-bromo-2-[(4-methoxyphenyl)methyl]-3,4-dihydro-2H-1λ\hat{ }6,2-benzothiazine-1,1-dione (400 mg, 1.04 mmol, 59.8%) as a yellow solid. LCMS: m/z 383 [M+H]⁺.

Step E. 6-((1-(tert-butyl)-3-((1S,3R)-3-((tert-butyldimethylsilyl)oxy)cyclopentyl)-1H-pyrazol-5-yl)amino)-2-(4-methoxybenzyl)-3,4-dihydro-2H-benzo[e][1,2]thiazine 1,1-dioxide To a stirred mixture of 6-bromo-2-[(4-methoxyphenyl)methyl]-3,4-dihydro-2H-1λ\hat{ }6, 2-benzothiazine-1,1-dione (400 mg, 1.04 mmol) in dioxane (15 mL) was added 1-tert-butyl-3-[(1S,3R)-3-[(tert-butyldimethylsilyl)oxy]cyclopentyl]-1H-pyrazol-5-amine (353 mg, 1.04 mmol), RuPhos Pd G2 (81.2 mg, 0.105 mmol), RuPhos (97.6 mg, 0.209 mmol) and Cs₂CO₃ (852. mg, 2.61 mmol) at rt. After stirred at 100° C. overnight, the mixture was poured into water (30 mL) and extracted with EtOAc (20 mL*2). The combined organic phase was washed with brine, dried with Na₂SO₄, filtered and concentrated. The residue was purified by chromatography (silica gel, 0-60%, EtOAc in PE) to give 6-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-3,4-dihydro-2H-1λ\hat{ }6,2-benzothiazine-1,1-dione (400 mg, 0.626 mmol, 60%) as a brown solid. LCMS: m/z 640 [M+H]⁺.

Step F. 6-((1-(tert-butyl)-3-((1S,3R)-3-hydroxycyclopentyl)-1H-pyrazol-5-yl)amino)-2-(4-methoxybenzyl)-3,4-dihydro-2H-benzo[e][1,2]thiazine 1,1-dioxide A mixture of 6-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-3,4-dihydro-2H-1λ\hat{ }6,2-benzothiazine-1,1-dione (400 mg, 0.626 mmol) in HCOOH (10 mL) was stirred at RT for 1 h, then concentrated under reduced pressure. The residue was treated with a mixture of LiOH (0.174 mL, 6.26 mmol) in EtOH (5 mL)/H₂O (5 mL). The resulting mixture was stirred at rt for 2 h, the mixture was concentrated under reduced pressure. The mixture was poured into water (30 mL) and extracted with EtOAc (20 mL*2). The combined organic phase was washed with brine, dried with Na₂SO₄, filtered and concentrated. The residue was purified by chromatography (silica gel, 0-8%, MeOH in DCM) to give 6-({1-tert-butyl-3-[(1S,3R)-3-hydroxycyclopentyl]-1H-pyrazol-5-yl}amino)-2-[(4-methoxyphenyl)methyl]-3,4-dihydro-2H-1λ\hat{ }6,2-benzothiazine-1,1-dione (300 mg, 0.572 mmol, 91.3%) as a white solid. LCMS: m/z 526 [M+H]⁺.

Step G. (1R,3S)-3-(1-(tert-butyl)-5-((2-(4-methoxybenzyl)-1,1-dioxido-3,4-dihydro-2H-benzo[e][1,2]thiazin-6-yl)amino)-1H-pyrazol-3-yl)cyclopentyl (4-nitrophenyl) carbonate To a stirred solution of 6-({1-tert-butyl-3-[(1S,3R)-3-hydroxycyclopentyl]-1H-pyrazol-5-yl}amino)-2-[(4-methoxyphenyl)methyl]-3,4-dihydro-2H-1λ\hat{ }6,2-benzothiazine-1,1-dione (180 mg, 0.343 mmol) in DCM (5 mL)/THF (5 mL) was added 4-nitrophenyl chloroformate (138 mg, 0.686 mmol), Pyridine (0.083 mL, 1.02 mmol) and DMAP (12.5 mg, 0.103 mmol) at rt. After stirred at rt for 3 h, the mixture was poured into water (30 ml) and extracted with EtOAc (20 ml*3). The combined organic phase was washed with brine, dried over Na₂SO₄, filtered and concentrated. The residue was purified by chromatography (silica gel, 0-10%, MeOH in DCM) to give (1R,3S)-3-[1-tert-butyl-5-({2-[(4-methoxyphenyl)methyl]-1,1-dioxo-3,4-dihydro-2H-1λ\hat{ }-1λ\hat{ }6,2-benzothiazin-6-yl}amino)-1H-pyrazol-3-yl]cyclopentyl 4-nitrophenyl carbonate (100 mg, 0.145 mmol, 42.2%) as a yellow solid. LCMS: m/z 691 [M+H]⁺.

Step H. (1R,3S)-3-(5-((1,1-dioxido-3,4-dihydro-2H-benzo[e][1,2]thiazin-6-yl)amino)-1H-pyrazol-3-yl)cyclopentyl (4-nitrophenyl) carbonate A mixture of (1R,3S)-3-[1-tert-butyl-5-({2-[(4-methoxyphenyl)methyl]-1,1-dioxo-3,4-dihydro-2H-1λ\hat{ }-1λ\hat{ }6,2-benzothiazin-6-yl}amino)-1H-pyrazol-3-yl]cyclopentyl 4-nitrophenyl carbonate (100 mg, 0.145 mmol) in HCOOH (5 mL) was stirred at 100° C. overnight, then concentrated under reduced pressure to give crude (1R,3S)-3-{5-[(1,1-dioxo-3,4-dihydro-2H-1λ\hat{ }6,2-benzothiazin-6-yl)amino]-1H-pyrazol-3-yl}cyclopentyl 4-nitrophenyl carbonate (70 mg, 0.136 mmol, 94.0%) as a brown oil. LCMS: m/z 515 [M+H]⁺.

Step I. (1R,3S)-3-(3-((1,1-dioxido-3,4-dihydro-2H-benzo[e][1,2]thiazin-6-yl)amino)-1H-pyrazol-5-yl)cyclopentyl isopropylcarbamate To a stirred solution of (1R,3S)-3-{5-[(1,1-dioxo-3,4-dihydro-2H-1λ\hat{ }-1λ\hat{ }6,2-benzothiazin-6-yl)amino]-1H-pyrazol-3-yl}cyclopentyl 4-nitrophenyl carbonate (70 mg, 0.136 mmol) in THF (5 mL) was added DIPEA (35.1 mg, 0.272 mmol) and propan-2-amine (0.058 mL, 0.680 mmol) slowly, the reaction mixture was stirred at room temperature for 2 h. The reaction was concentrated. The residue was purified by prep-HPLC (C18, 40˜ 90% MeCN in H₂O with 0.1% HCOOH) to give (1R,3S)-3-{3-[(1,1-dioxo-3,4-dihydro-2H-11λ\hat{ }6,2-benzothiazin-6-yl)amino]-1H-pyrazol-5-yl}cyclopentyl N-(propan-2-yl)carbamate (22.8 mg, 0.053 mmol, 38.9%) as a white solid. LCMS: m/z 434 [M+H]⁺. ¹H NMR (400 MHz, DMSO) δ 8.82 (s, 1H), 7.48 (d, J=8.4 Hz, 1H), 7.26-7.14 (m, 2H), 7.15 (brs, 1H), 6.95 (d, J=7.2 Hz, 1H), 5.69 (s, 1H), 4.99 (brs, 1H), 3.65-3.50 (m, 1H), 3.50 (d, J=4.8 Hz, 2H), 3.11-2.99 (m, 1H), 2.81 (t, J=6.0 Hz, 2H), 2.49-2.33 (m, 1H), 2.12-1.98 (m, 1H), 1.94-1.85 (m, 1H), 1.83-1.67 (m, 2H), 1.65-1.54 (m, 1H), 1.03 (d, J=6.4 Hz, 6H).

### Example 3: (1R,3S)-3-(3-((1,1-dioxido-2,3-dihydrobenzo[d]isothiazol-5-yl)amino)-1H-pyrazol-5-yl)cyclopentyl isopropylcarbamate

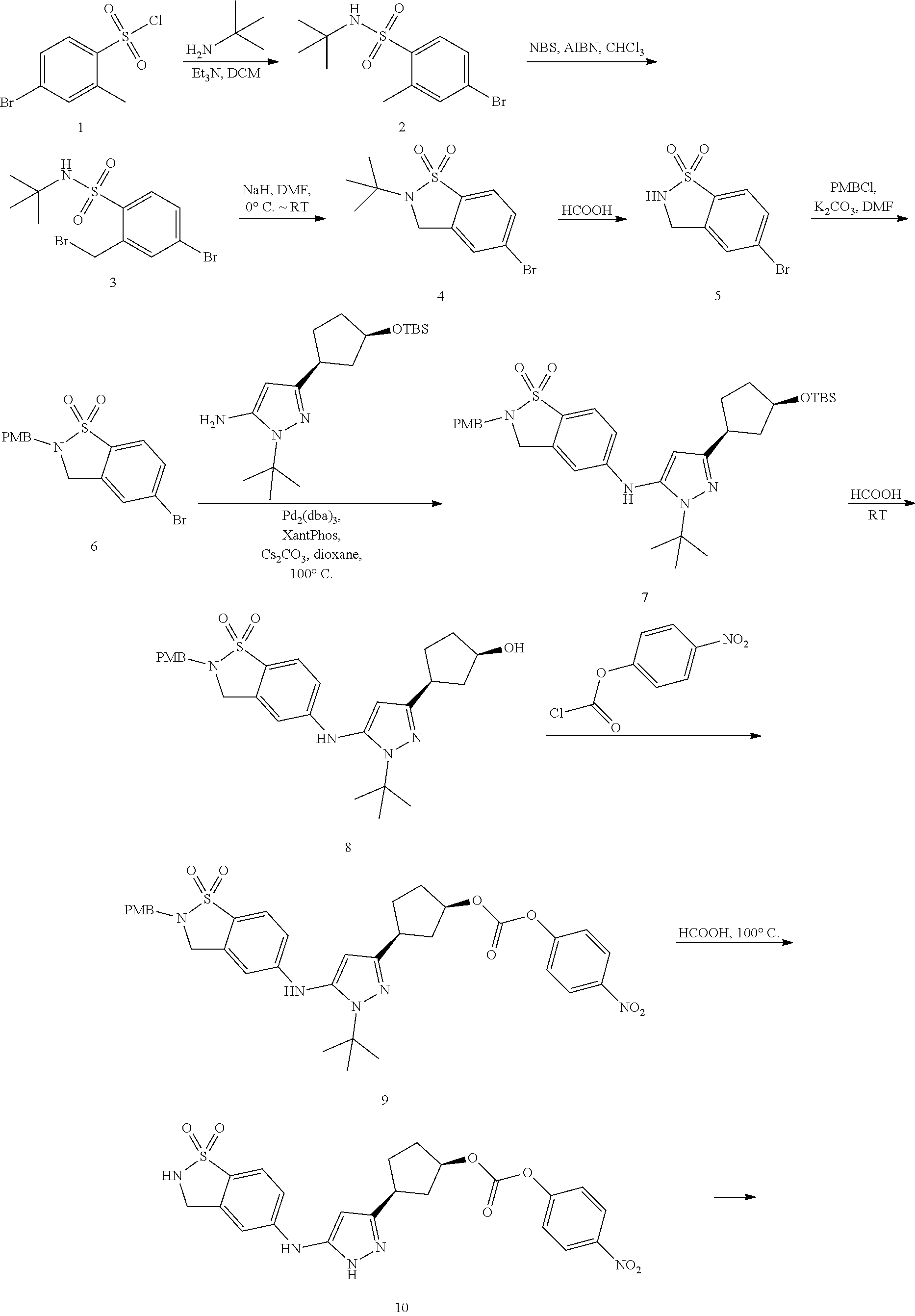

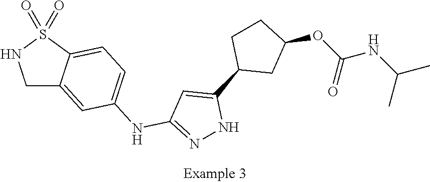 [SMILES: CC(C)NC(=O)O[C@@H]1CC[C@H](c2cc(Nc3ccc4c(c3)CNS4(=O)=O)n[nH]2)C1]

Step A: 4-bromo-N-(tert-butyl)-2-methylbenzenesulfonamide To a solution of 4-bromo-2-methylbenzene-1-sulfonyl chloride (5 g, 18.5 mmol) in DCM (20 mL) were added triethylamine (7.73 mL, 55.6 mmol), 2-methylpropan-2-amine (3.93 mL, 37.1 mmol) and the reaction was stirred at room temperature for overnight. The reaction was concentrated in vacuo. The residue was purified using silica gel column chromatography eluting with [gradient: 0-50%] to afford the title compound 4-bromo-N-tert-butyl-2-methylbenzene-1-sulfonamide (5 g, 16.3 mmol, 88.0%) as a white solid. LCMS: ESI m/z 306 [M+H]⁺. ¹H NMR (400 MHz, DMSO) δ 7.75 (d, J=8.4 Hz, 1H), 7.65-7.49 (m, 3H), 2.54 (s, 3H), 1.06 (s, 9H).

Step B: 4-bromo-2-(bromomethyl)-N-(tert-butyl)benzenesulfonamide To a solution of 4-bromo-N-tert-butyl-2-methylbenzene-1-sulfonamide (5 g, 16.3 mmol) in CHCl₃ (10 mL) were added AIBN (268 mg, 1.63 mmol), DIEA (0.448 mL, 2.71 mmol), and NBS (2.91 g, 16.3 mmol), and the reaction was stirred at 60° C. overnight. The reaction was concentrated. The residue was purified using silica gel column chromatography eluting with [gradient: 0-30%] to afford the title compound 4-bromo-2-(bromomethyl)-N-tert-butylbenzene-1-sulfonamide (1.9 g, 4.9 mmol, 30.2%) as a white solid. LCMS: ESI m/z 384 [M+H]⁺. ¹H NMR (400 MHz, DMSO) δ 7.91-7.86 (m, 2H), 7.82 (d, J=8.4 Hz, 1H), 7.74-7.68 (m, 1H), 5.03 (s, 2H), 1.13 (s, 9H).

Step C: 5-bromo-2-(tert-butyl)-2,3-dihydrobenzo[d]isothiazole 1,1-dioxide To a solution of 4-bromo-2-(bromomethyl)-N-tert-butylbenzene-1-sulfonamide (1.4 g, 3.63 mmol) in MeOH (20 mL) and H₂O (5 mL) was added NaOH (0.44 g, 10.9 mmol) and the reaction was stirred at room temperature for 3 hr. The reaction was concentrated in vacuo. The residue was purified using silica gel column chromatography eluting with EA in PE [gradient: 0-80%] to afford the title compound 5-bromo-2-tert-butyl-2,3-dihydro-λ^{A}6,2-benzothiazole-1,1-dione (900 mg, 2.95 mmol, 81.4%) as a white solid. LCMS: ESI m/z 304 [M+H]⁺. ¹H NMR (400 MHz, DMSO) δ 7.83 (d, J=0.8 Hz, 1H), 7.77 (dt, J=14.4, 4.8 Hz, 2H), 4.55 (s, 2H), 1.46 (s, 9H).

Step D: 5-bromo-2,3-dihydrobenzo[d]isothiazole 1,1-dioxide To a solution of 5-bromo-2-tert-butyl-2,3-dihydro-1λ\hat{ }6,2-benzothiazole-1,1-dione (900 mg, 2.95 mmol) in HCOOH (10 mL) and the reaction was stirred at room temperature for 1 hr. The reaction was concentrated and were added MeOH (10 mL), and LiOH (496 mg, 11.8 mmol), and the reaction was stirred at room temperature for 30 min. The reaction was diluted with H₂O. The aqueous layer was extracted with EA. Combined the EA layer and washed with saturated NaCl solution. The EA layer was dried by Na₂SO₄, filtered and concentrated. The residue was purified using silica gel column chromatography eluting with [gradient: 0-100%] to afford the title compound 5-bromo-2,3-dihydro-1λ\hat{ }6,2-benzothiazole-1,1-dione (600 mg, 2.41 mmol, 81.7%) as a white solid. LCMS: ESI m/z 248 [M+H]⁺. ¹H NMR (400 MHz, DMSO) δ 7.92 (s, 1H), 7.85-7.73 (m, 3H), 4.40 (s, 2H).

Step E: 5-bromo-2-(4-methoxybenzyl)-2,3-dihydrobenzo[d]isothiazole 1,1-dioxide To a solution of 5-bromo-2,3-dihydro-1λ\hat{ }6,2-benzothiazole-1,1-dione (500 mg, 2.01 mmol) in DMF (10 mL) were added 1-(chloromethyl)-4-methoxybenzene (0.412 mL, 3.023 mmol), and Cs₂CO₃ (1.97 g, 6.04 mmol), and the reaction was stirred at room temperature for overnight. The reaction was diluted with H₂O. The aqueous layer was extracted with EA. Combined the EA layer and washed with saturated NaCl solution. The EA layer was dried by Na₂SO₄, filtered and concentrated. The residue was purified using silica gel column chromatography eluting with [gradient: 0-30%] to afford the title compound 5-bromo-2-[(4-methoxyphenyl)methyl]-2,3-dihydro-1λ\hat{ }6,2-benzothiazole-1,1-dione (350 mg, 0.950 mmol, 47.1%) as a white solid. LCMS: ESI m/z 368 [M+H]⁺. ¹H NMR (400 MHz, DMSO) δ 7.90-7.82 (m, 2H), 7.80 (dd, J=8.4, 1.6 Hz, 1H), 7.42-7.32 (m, 2H), 6.99-6.91 (m, 2H), 4.33 (s, 2H), 4.27 (s, 2H), 3.76 (s, 3H).

Step F: 5-((1-(tert-butyl)-3-((1S,3R)-3-((tert-butyldimethylsilyl)oxy)cyclopentyl)-1H-pyrazol-5-yl)amino)-2-(4-methoxybenzyl)-2,3-dihydrobenzo[d]isothiazole 1,1-dioxide To a solution of 5-bromo-2-[(4-methoxyphenyl)methyl]-2,3-dihydro-1λ\hat{ }6,2-benzothiazole-1,1-dione (350 mg, 0.950 mmol) in dioxane (10 mL) were added 1-tert-butyl-3-[(1S,3R)-3-[(tert-butyldimethylsilyl)oxy]cyclopentyl]-1H-pyrazol-5-amine (353 mg, 1.04 mmol), Pd₂(dba)₃ (87.1 mg, 95.1 μmol), Xantphos (110 mg, 0.190 mmol) and Cs₂CO₃ (619 mg, 1.90 mmol). The reaction was stirred at 100° C. for 6 h. The reaction was diluted with EA and water. The organic layer was separated, washed with saturated NaCl solution and concentrated in vacuo. The residue was purified using silica gel column chromatography elutied with ethyl acetate in petroleum ether (gradient:0-50%) to afford the title compound 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-1λ\hat{ }6,2-benzothiazole-1,1-dione (500 mg, 0.800 mmol, 84.2%) as a white solid. LCMS: ESI m/z 625 [M+H]⁺

Step G: 5-((1-(tert-butyl)-3-((1S,3R)-3-hydroxycyclopentyl)-1H-pyrazol-5-yl)amino)-2-(4-methoxybenzyl)-2,3-dihydrobenzo[d]isothiazole 1,1-dioxide A solution of 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-11λ\hat{ }6,2-benzothiazole-1,1-dione (500 mg, 0.800 mmol) in HCOOH (10 mL) was stirred at rt for 1 h. LCMS showed the reaction had been completed. The solvent was removed under vacuum. The residue was purified by silica gel chromatography (eluting with 50% ethyl acetate in petroleum ether) to give 5-({1-tert-butyl-3-[(1S,3R)-3-hydroxycyclopentyl]-1H-pyrazol-5-yl}amino)-2-[(4-methoxyphenyl)methyl]-2,3-dihydro-1λ\hat{ }6,2-benzothiazole-1,1-dione (400 mg, 0.697 mmol, 87.1%) as a yellow solid. LCMS: ESI m/z 511 [M+H]⁺

Step H: (1R,3S)-3-(1-(tert-butyl)-5-((2-(4-methoxybenzyl)-1,1-dioxido-2,3-dihydrobenzo[d]isothiazol-5-yl)amino)-1H-pyrazol-3-yl)cyclopentyl (4-nitrophenyl) carbonate To a solution of 5-({1-tert-butyl-3-[(1S,3R)-3-hydroxycyclopentyl]-1H-pyrazol-5-yl}amino)-2-[(4-methoxyphenyl)methyl]-2,3-dihydro-1λ\hat{ }6,2-benzothiazole-1,1-dione (400 mg, 0.783 mmol) in THF/DCM(1:1) (12 mL) was added Pyridine (0.190 mL, 2.35 mmol), DMAP (9.57 mg, 78.1 μmmol) and 4-nitrophenyl chloroformate (316 mg, 1.57 mmol). The mixture was stirred at room temperature for 4 h. The solvents were removed under vacuum. The residue was dissolved in ethyl acetate (5 mL*3), washed with sat. aq. NaCl, dried over sodium sulfate, filtered, concentrated, and purified by silica gel chromatography (eluting with 50% ethyl acetate in petroleum ether) to give(1R,3S)-3-[1-tert-butyl-5-({2-[(4-methoxyphenyl)methyl]-1,1-dioxo-2,3-dihydro-1λ\hat{ }6,2-benzothiazol-5-yl}amino)-1H-pyrazol-3-yl]cyclopentyl 4-nitrophenyl carbonate (300 mg, 0.444 mmol, 56.7%) as a yellow solid. LCMS: ESI m/z 676 [M+H]⁺

Step I: (1R,3S)-3-(5-((1,1-dioxido-2,3-dihydrobenzo[d]isothiazol-5-yl)amino)-1H-pyrazol-3-yl)cyclopentyl (4-nitrophenyl) carbonate A solution of (1R,3S)-3-[1-tert-butyl-5-({2-[(4-methoxyphenyl)methyl]-1,1-dioxo-2,3-dihydro-1λ\hat{ }6,2-benzothiazol-5-yl}amino)-1H-pyrazol-3-yl]cyclopentyl 4-nitrophenyl carbonate (300 mg, 0.444 mmol) in HCOOH (8 mL) was stirred at 90° C. overnight. The reaction was complete detected by LCMS. The residue was concentrated to afford (1R,3S)-3-{5-[(1,1-dioxo-2,3-dihydro-1λ\hat{ }6,2-benzothiazol-5-yl)amino]-1H-pyrazol-3-yl}cyclopentyl 4-nitrophenyl carbonate (150 mg, 0.300 mmol, 67.6%) as a yellow oil. LCMS: ESI m/z 500 [M+H]⁺

Step J: (1R,3S)-3-(3-((1,1-dioxido-2,3-dihydrobenzo[d]isothiazol-5-yl)amino)-1H-pyrazol-5-yl)cyclopentyl isopropylcarbamate A solution of of (1R,3S)-3-{5-[(1,1-dioxo-2,3-dihydro-11λ\hat{ }6,2-benzothiazol-5-yl)amino]-1H-pyrazol-3-yl}cyclopentyl 4-nitrophenyl carbonate (150 mg, 0.300 mmol) in propan-2-amine (10 mL, 117 mmol) was stirred at rt for 3 h. The mixture was concentrated under vacuum. The residue was purified by prep-HPLC to give(1R,3S)-3-{3-[(1,1-dioxo-2,3-dihydro-1λ\hat{ }6,2-benzothiazol-5-yl)amino]-1H-pyrazol-5-yl}cyclopentyl N-(propan-2-yl)carbamate (30 mg, 72.2 μmmol, 23.8%) as a yellow solid. LCMS: ESI m/z 420 [M+H]⁺. ¹H NMR (400 MHz, DMSO) δ 9.02 (s, 1H), 7.53 (d, J=8.4 Hz, 1H), 7.45 (s, 1H), 7.30 (dd, J=8.6, 1.8 Hz, 1H), 6.95 (d, J=7.6 Hz, 1H), 5.72 (s, 1H), 4.99 (brs, 1H), 4.29 (s, 1H), 3.62-3.48 (m, 1H), 3.15-2.96 (m, 1H), 2.48-2.40 (m, 1H), 2.08-1.94 (m, 1H), 1.93-1.81 (m, 1H), 1.78-1.66 (m, 2H), 1.65-1.52 (m, 1H), 1.03 (d, J=6.4 Hz, 6H).

### Example 4: (1R,3S)-3-(3-((1,3-dioxoisoindolin-5-yl)amino)-1H-pyrazol-5-yl)cyclopentyl isopropylcarbamate

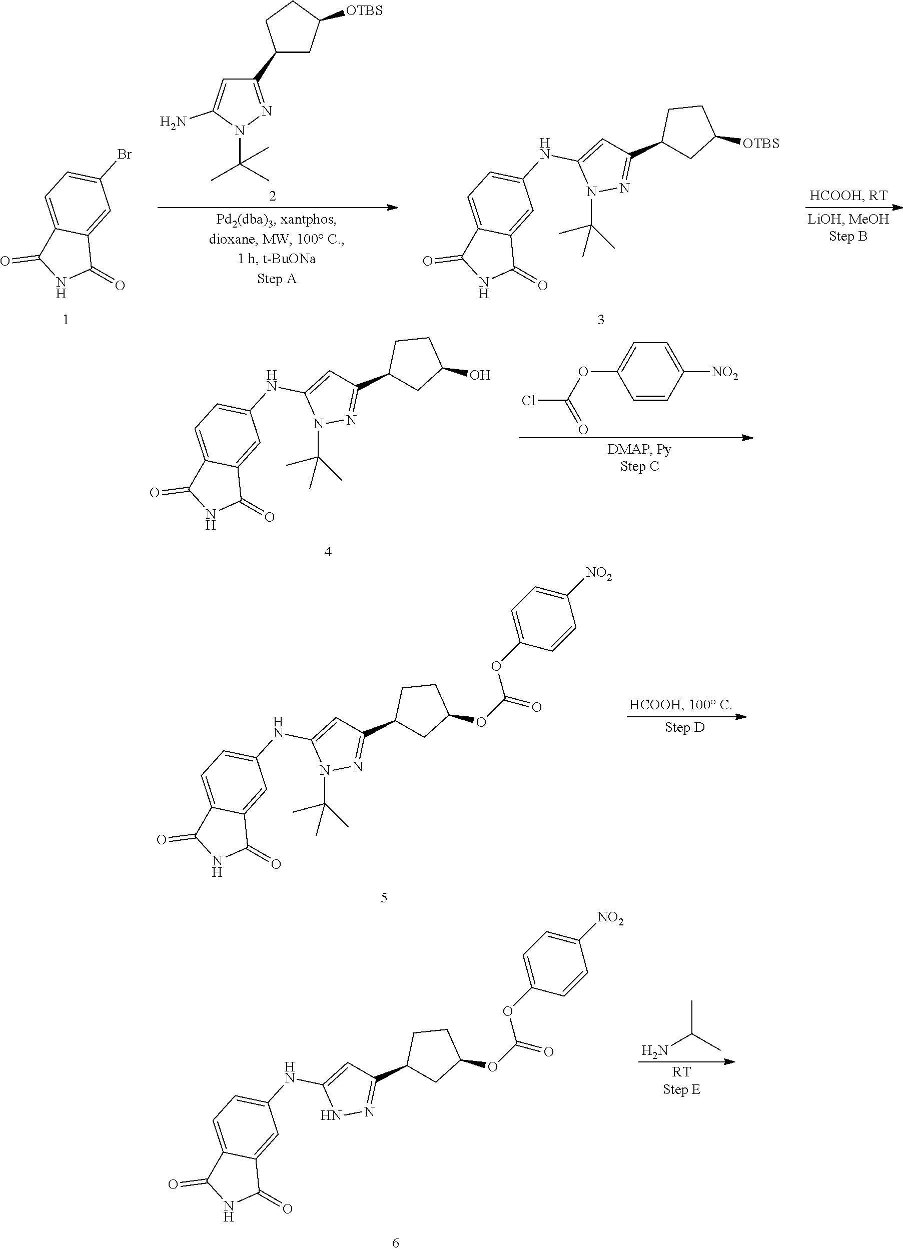

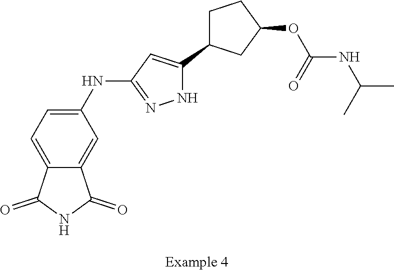 [SMILES: CC(C)NC(=O)O[C@@H]1CC[C@H](c2cc(Nc3ccc4c(c3)C(=O)NC4=O)n[nH]2)C1]

Step A: 5-((1-(tert-butyl)-3-((1S,3R)-3-((tert-butyldimethylsilyl)oxy)cyclopentyl)-1H-pyrazol-5-yl)amino)isoindoline-1,3-dione To a solution of 1-tert-butyl-3-[(1S,3R)-3-[(tert-butyldimethylsilyl)oxy]cyclopentyl]-1H-pyrazol-5-amine (300 mg, 0.889 mmol) in dioxane (2 mL) were added 5-bromo-2,3-dihydro-1H-isoindole-1,3-dione (200 mg, 0.889 mmol), Xant-PHOS (102 mg, 0.178 mmol), sodium 2-methylpropan-2-olate (1.77 mL, 1.778 mmol) and Pd₂(dba)₃ (81.4 mg, 0.089 mmol). The reaction was stirred at 100° C. under N₂ using M.W for 1 hr. The cooled reaction mixture was concentrated in vacuo. The residue was purified using silica gel column chromatography eluting with EA in PE [gradient: 0-100%]. The organic layer was collected, concentrated in vacuo, and dried to afford the title compound 5-({1-tert-butyl-3-[(1S,3R)-3-[(tert-butyldimethylsilyl)oxy]cyclopentyl]-1H-pyrazol-5-yl}amino)-2,3-dihydro-1H-isoindole-1,3-dione (240 mg, 0.497 mmol, 55.9%) as a white solid. LCMS: ESI m/z 483 [M+H]⁺

Step B: 5-((1-(tert-butyl)-3-((1S,3R)-3-hydroxycyclopentyl)-1H-pyrazol-5-yl)amino)isoindoline-1,3-dione A solution of 5-({1-tert-butyl-3-[(1S,3R)-3-[(tert-butyldimethylsilyl)oxy]cyclopentyl]-1H-pyrazol-5-yl}amino)-2,3-dihydro-1H-isoindole-1,3-dione (240 mg, 0.497 mmol) in HCOOH (3 mL) was stirred at room temperature for 1 hr. The reaction was concentrated and were added MeOH (3 mL), and LiOH (41.7 mg, 0.994 mmol). The following reaction mixture was stirred at room temperature for 30 min. The reaction was diluted with H₂O. The aqueous layer was extracted with EA. Combined the EA layer and washed with saturated NaCl solution. The EA layer was dried by Na₂SO₄, filtered and concentrated. The residue was purified using silica gel column chromatography eluting with [gradient: 0-100%] to afford the title compound 5-({1-tert-butyl-3-[(1S,3R)-3-hydroxycyclopentyl]-1H-pyrazol-5-yl}amino)-2,3-dihydro-1H-isoindole-1,3-dione (50 mg, 0.136 mmol, 27.2%) as a white solid. LCMS: ESI m/z 387 [M+H]⁺

Step C: (1R,3S)-3-(1-(tert-butyl)-5-((1,3-dioxoisoindolin-5-yl)amino)-1H-pyrazol-3-yl)cyclopentyl (4-nitrophenyl) carbonate To a solution of 5-({1-tert-butyl-3-[(1S,3R)-3-hydroxycyclopentyl]-1H-pyrazol-5-yl}amino)-2,3-dihydro-1H-isoindole-1,3-dione (50 mg, 0.136 mmol) in DCM (2 mL) were added 4-nitrophenyl chloroformate (41. mg, 0.204 mmol), DMAP (1.66 mg, 0.014 mmol), pyridine (0.022 mL, 0.271 mmol). The reaction was stirred at 40° C. for 3 hr. The reaction was and concentrated in vacuo. The residue was purified using silica gel column chromatography eluting with EA inPE [Gradient: 0-100%] to afford the title compound (1R,3S)-3-{1-tert-butyl-5-[(1,3-dioxo-2,3-dihydro-1H-isoindol-5-yl)amino]-1H-pyrazol-3-yl}cyclopentyl 4-nitrophenyl carbonate (27 mg, 0.051 mmol, 37.2%) as a white solid. LCMS: ESI m/z 534 [M+H]⁺

Step D: (1R,3S)-3-(5-((1,3-dioxoisoindolin-5-yl)amino)-1H-pyrazol-3-yl)cyclopentyl (4-nitrophenyl) carbonate A solution of (1R,3S)-3-{1-tert-butyl-5-[(1,3-dioxo-2,3-dihydro-1H-isoindol-5-yl)amino]-1H-pyrazol-3-yl}cyclopentyl 4-nitrophenyl carbonate (27 mg, 0.051 mmol) in HCOOH (3 mL) was stirred at 100° C. overnight. The cooled reaction mixture was concentrated in vacuo to afford the title compound (1R,3S)-3-{5-[(1,3-dioxo-2,3-dihydro-1H-isoindol-5-yl)amino]-1H-pyrazol-3-yl}cyclopentyl 4-nitrophenyl carbonate (20 mg, 0.042 mmol, 82.8%) as a white solid. LCMS: ESI m/z 478 [M+H]⁺

Step E: (1R,3S)-3-(3-((1,3-dioxoisoindolin-5-yl)amino)-1H-pyrazol-5-yl)cyclopentyl isopropylcarbamate A solution of (1R,3S)-3-{5-[(1,3-dioxo-2,3-dihydro-1H-isoindol-5-yl)amino]-1H-pyrazol-3-yl}cyclopentyl 4-nitrophenyl carbonate (20 mg, 0.042 mmol) in propan-2-amine (2 mL) was stirred at RT for 2 hr. The reaction concentrated in vacuo. The residue was purified by prep-HPLC to afford the title compound 5-({3-[(1S,3R)-3-{[(propan-2-yl)amino]oxy}cyclopentyl]-1H-pyrazol-5-yl}amino)-2,3-dihydro-1H-isoindole-1,3-dione (3.2 mg, 0.009 mmol, 20.7%) as a yellow solid. LCMS: ESI m/z 398 [M+H]⁺. ¹H NMR (400 MHz, DMSO) δ 12.01 (s, 1H), 10.86 (s, 1H), 9.34 (s, 1H), 7.87 (s, 1H), 7.56 (dd, J=29.2, 8.4 Hz, 2H), 6.95 (d, J=7.2 Hz, 1H), 5.72 (s, 1H), 5.01 (s, 1H), 3.75-3.53 (m, 1H), 3.12-3.04 (m, 1H), 2.09-2.00 (m, 1H), 1.96-1.86 (m, 1H), 1.82-1.64 (m, 2H), 1.64-1.53 (m, 1H), 1.24 (s, 1H), 1.04 (d, J=6.4 Hz, 6H).

### Example 5: (1R,3S)-3-(5-((1,1-dioxido-3-oxo-2,3-dihydrobenzo[d]isothiazol-5-yl)amino)-1H-pyrazol-3-yl)cyclopentyl isopropylcarbamate

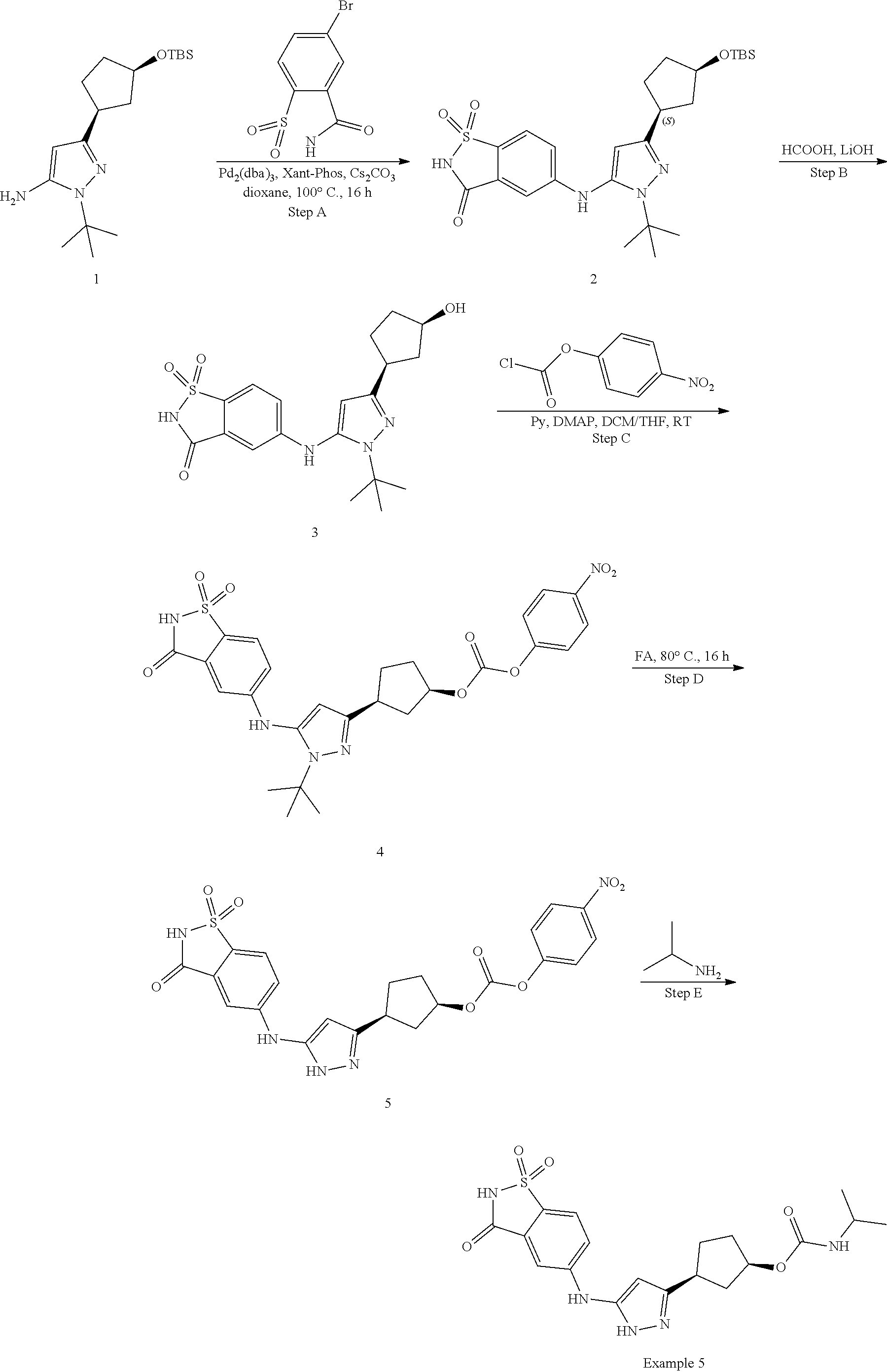

Step A: 5-((1-(tert-butyl)-3-((1S,3R)-3-((tert-butyldimethylsilyl)oxy)cyclopentyl)-1H-pyrazol-5-yl)amino)benzo[d]isothiazol-3(2H)-one 1,1-dioxide To a solution of 1-tert-butyl-3-[(1S,3R)-3-[(tert-butyldimethylsilyl)oxy]cyclopentyl]-1H-pyrazol-5-amine (500 mg, 1.48 mmol) in dioxane (2 mL) were added 5-bromo-2,3-dihydro-1λ\hat{ }6,2-benzothiazole-1,1,3-trione (582 mg, 2.22 mmol), Xant-PHOS (171 mg, 0.296 mmol), Cs₂CO₃ (482 mg, 1.481 mmol) and Pd₂(dba)₃ (135 mg, 0.148 mmol). The reaction mixture was stirred at 100° C. under N₂ for 16 hr. The cooled reaction mixture was diluted with EA and water. The organic layer was separated, washed with further brine, dried over Na₂SO₄ and concentrated. The residue was purified using silica gel column chromatography eluting with ethyl acetate in petroleum ether (3/1) The organic layer was collected, concentrated in vacuo, and dried to afford the title compound 5-({1-tert-butyl-3-[(1S,3R)-3-[(tert-butyldimethylsilyl)oxy]cyclopentyl]-1H-pyrazol-5-yl}amino)-2,3-dihydro-1λ\hat{ }6,2-benzothiazole-1,1,3-trione (500 mg, 0.964 mmol, 65.0%) as a yellow solid. LCMS: ESI 519 [M+H]+

Step B: 5-((1-(tert-butyl)-3-((1S,3R)-3-hydroxycyclopentyl)-1H-pyrazol-5-yl)amino)benzo[d]isothiazol-3(2H)-one 1,1-dioxide A solution of 5-({1-tert-butyl-3-[(1S,3R)-3-[(tert-butyldimethylsilyl)oxy]cyclopentyl]-1H-pyrazol-5-yl}amino)-2,3-dihydro-1λ\hat{ }6,2-benzothiazole-1,1,3-trione (500 mg, 0.964 mmol) in HCOOH (5 mL) was stirred at room temperature for 1 hr. The reaction was concentrated and were added MeOH (50 mL), and LiOH (161 mg, 3.85 mmol), and the reaction was stirred at room temperature for 30 min. The reaction was diluted with H₂O. The aqueous layer was extracted with EA. Combined the EA layer and washed with brine. The EA layer was dried by anhydrous Na₂SO₄, filtered and concentrated. The residue was purified using silica gel column chromatography eluting with [gradient: 0-100%] to afford the title compound 5-({1-tert-butyl-3-[(1S,3R)-3-hydroxycyclopentyl]-1H-pyrazol-5-yl}amino)-2,3-dihydro-1λ\hat{ }6,2-benzothiazole-1,1,3-trione (300 mg, 0.742 mmol, 76.9%) as a white solid. LCMS: ESI m/z 405 [M+H]⁺

Step C: (1R,3S)-3-(1-(tert-butyl)-5-((1,1-dioxido-3-oxo-2,3-dihydrobenzo[d]isothiazol-5-yl)amino)-1H-pyrazol-3-yl)cyclopentyl (4-nitrophenyl) carbonate To a solution of 5-({1-tert-butyl-3-[(1S,3R)-3-hydroxycyclopentyl]-1H-pyrazol-5-yl}amino)-2,3-dihydro-1λ\hat{ }6,2-benzothiazole-1,1,3-trione (300 mg) in THF (10 mL) and DCM (10 mL) was added 4-nitrophenyl chloroformate (568 mg, 2.82 mmol) and pyridine (0.456 mL, 5.64 mmol). The reaction was stirred at 50° C. for 30 min. The reaction was concentrated in vacuo. The residue was purified using silica gel column chromatography eluting with EA in PE [gradient: 80-100%] to afford the title compound (1R,3S)-3-{1-tert-butyl-5-[(1,1,3-trioxo-2,3-dihydro-11λ\hat{ }6,2-benzothiazol-5-yl)amino]-1H-pyrazol-3-yl}cyclopentyl 4-nitrophenyl carbonate (200 mg, 0.351 mmol, 18.7%) as a white solid. LCMS: ESI m/z 570 [M+H]⁺

Step D: (1R,3S)-3-(5-((1,1-dioxido-3-oxo-2,3-dihydrobenzo[d]isothiazol-5-yl)amino)-1H-pyrazol-3-yl)cyclopentyl (4-nitrophenyl) carbonate A solution of (1R,3S)-3-{1-tert-butyl-5-[(1,1,3-trioxo-2,3-dihydro-1λ\hat{ }6,2-benzothiazol-5-yl)amino]-1H-pyrazol-3-yl}cyclopentyl 4-nitrophenyl carbonate (200 mg) in HCOOH (5 mL) was stirred at 80° C. overnight. The reaction was concentrated in vacuo to afford the title compound 4-nitrophenyl (1R,3S)-3-(5-((1,1-dioxido-3-oxo-2,3-dihydrobenzo[d]isothiazol-5-yl)amino)-1H-pyrazol-3-yl)cyclopentyl (4-nitrophenyl) carbonate (100 mg) as a white solid, which was used into next step without further purification. LCMS: ESI m/z 514 [M+H]⁺

Step E: (1R,3S)-3-(5-((1,1-dioxido-3-oxo-2,3-dihydrobenzo[d]isothiazol-5-yl)amino)-1H-pyrazol-3-yl)cyclopentyl isopropylcarbamate To a solution of 4-nitrophenyl (1R,3S)-3-{5-[(1,1,3-trioxo-2,3-dihydro-1λ\hat{ }6,2-benzothiazol-5-yl)amino]-1H-pyrazol-3-yl}cyclopentyl carbonate (100 mg, 0.195 mmol) in THF (2 mL) was added propan-2-amine (0.167 mL, 1.95 mmol) and the reaction was stirred at 50° C. for 2 hr. The reaction concentrated in vacuo. The residue was purified by prep-HPLC (0.1% TFA) to afford the title compound (1R,3S)-3-{5-[(1,1,3-trioxo-2,3-dihydro-1λ\hat{ }6,2-benzothiazol-5-yl)amino]-1H-pyrazol-3-yl}cyclopentyl N-(propan-2-yl)carbamate (10.4 mg) as a white solid. LCMS: ESI m/z 434 [M+H]⁺. ¹H NMR (400 MHz, DMSO-d₆) δ 12.05 (s, 1H), 9.46 (s, 1H), 8.03 (d, J=2.0 Hz, 1H), 7.86 (d, J=8.8 Hz, 1H), 7.65 (dd, J=8.8, 2.0 Hz, 1H), 6.95 (d, J=7.6 Hz, 1H), 5.72 (s, 1H), 5.00 (s, 1H), 3.11-3.04 (m, 1H), 2.13-1.55 (m, 6H), 1.03 (d, J=8.0 Hz, 6H).

### Example 6: (1R,3S)-3-(3-((1,1-dioxido-3,4-dihydro-2H-benzo[b][1,4,5]oxathiazepin-7-yl)amino)-1H-pyrazol-5-yl)cyclopentyl isopropylcarbamate

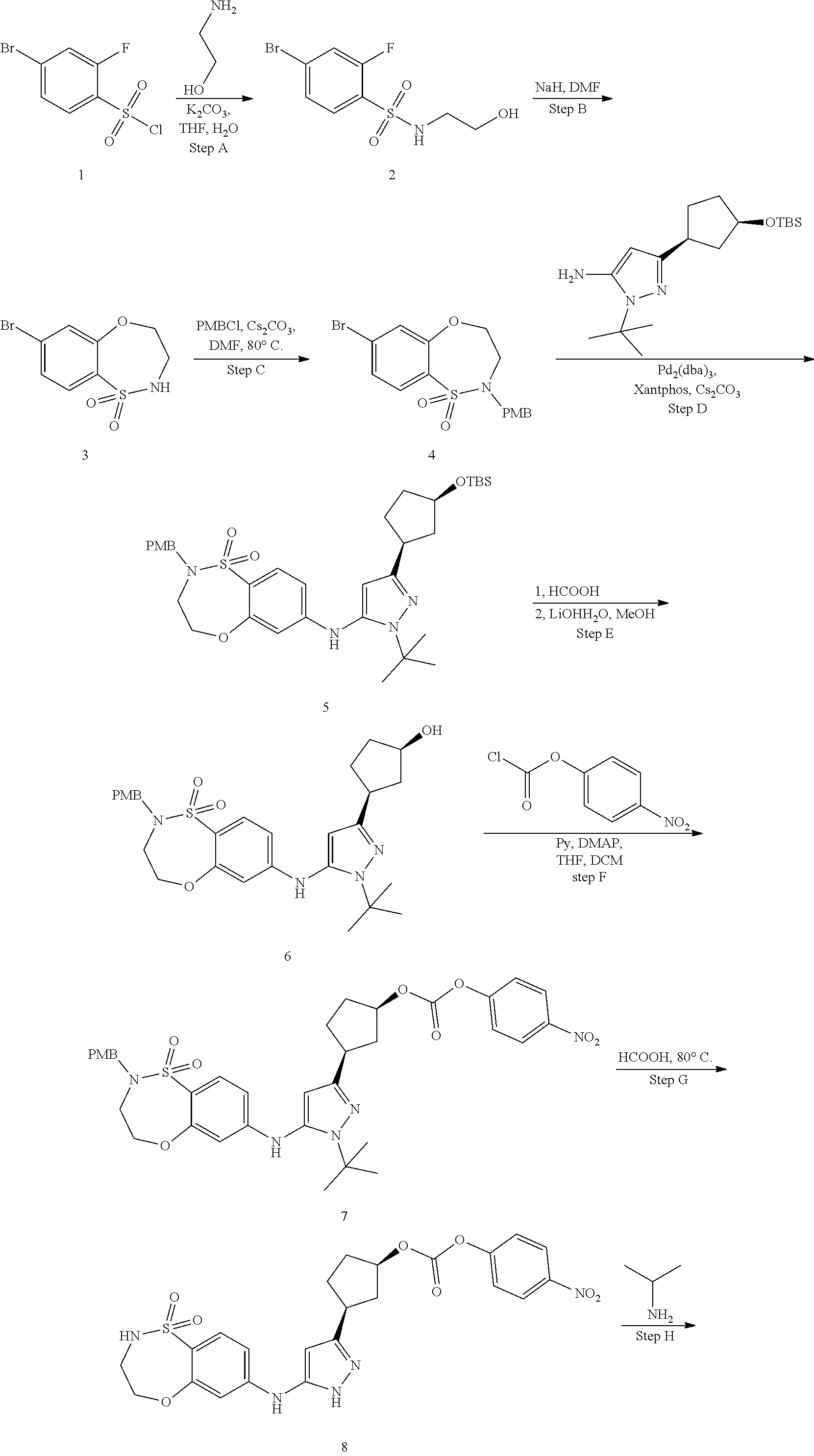

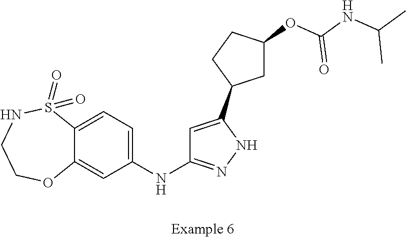 [SMILES: CC(C)NC(=O)O[C@@H]1CC[C@H](c2cc(Nc3ccc4c(c3)OCCNS4(=O)=O)n[nH]2)C1]

Step A: 4-bromo-2-fluoro-N-(2-hydroxyethyl)benzenesulfonamide To a solution of 2-aminoethan-1-ol (2.2 mL, 36.6 mmol), K₂CO₃ (5.05 g, 36.5 mmol) in THF (200 mL) and H₂O (50 mL) was added 4-bromo-2-fluorobenzene-1-sulfonyl chloride (10 g, 36.6 mmol) in portion. The mixture was stirred at rt for 2 h, then diluted with EA and aq·NaCl. The organic layer was separated, washed with further brine, and concentrated in vacuo to afford the title compound S-(4-bromo-2-fluorophenyl)-2-hydroxyethane-1-sulfonamido (10 g, 33.5 mmol, 91.7%) as a white solid.

Step B: 7-bromo-3,4-dihydro-2H-benzo[b][1,4,5]oxathiazepine 1,1-dioxide To a solution of S-(4-bromo-2-fluorophenyl)-2-hydroxyethane-1-sulfonamido (5 g, 16.8 mmol) in DMF (50 mL) was added NaH (1.48 g, 0.370 mmol, 60% in mineral oil) at 0° C. The reaction was stirred at RT overnight. The reaction was diluted with EA and H₂O, adjusted pH=6 with 1M HCl. The organic layer was separated, washed with further brine, and concentrated in vacuo. The residue was purified using silica gel column chromatography eluting with EA in PE [Gradient: 0-50%] to afford the title compound 7-bromo-3,4-dihydro-2H-5, 1λ\hat{ }6,2-benzoxathiazepine-1,1-dione (1.3 g, 4.67 mmol, 27.9%) as a colorless oil. LCMS: ESI m/z 280 [M+2+H]⁺

Step C: 7-bromo-2-(4-methoxybenzyl)-3,4-dihydro-2H-benzo[b][1,4,5]oxathiazepine 1,1-dioxide To a solution of 7-bromo-3,4-dihydro-2H-5,1λ\hat{ }6,2-benzoxathiazepine-1,1-dione (690 mg, 2.48 mmol) and Cs₂CO₃ (1.62 g, 4.96 mmol) in DMF (10 mL) was added 1-(chloromethyl)-4-methoxybenzene (0.5 mL, 3.72 mmol) at 0° C. The reaction was stirred at 80° C. for 3 h. The reaction was diluted with EA and H₂O. The organic layer was separated, washed with further brine, and concentrated in vacuo. The residue was purified using silica gel column chromatography eluting with EA in PE [Gradient: 0-50%] to afford the title compound 7-bromo-2-[(4-methoxyphenyl)methyl]-3,4-dihydro-2H-5,1λ\hat{ }6,2-benzoxathiazepine-1,1-dione (900 mg, 2.26 mmol, 91.1%) as a colorless oil.

Step D: 7-((1-(tert-butyl)-3-((1S,3R)-3-((tert-butyldimethylsilyl)oxy)cyclopentyl)-1H-pyrazol-5-yl)amino)-2-(4-methoxybenzyl)-3,4-dihydro-2H-benzo[b][1,4,5]oxathiazepine 1,1-dioxide To a solution of 7-bromo-2-[(4-methoxyphenyl)methyl]-3,4-dihydro-2H-5,1λ\hat{ }6,2-benzoxathiazepine-1,1-dione (650 mg, 1.63 mmol) in dioxane (15 mL) were added 1-tert-butyl-3-[(3R)-3-[(tert-butyldimethylsilyl)oxy]cyclopentyl]-1H-pyrazol-5-amine (551 mg, 1.63 mmol), Pd₂(dba)₃ (149 mg, 0.163 mmol), Xantphos (189 mg, 0.326 mmol) and Cs₂CO₃ (1.60 g, 4.90 mmol). The reaction was stirred at 100° C. under N₂ overnight. The reaction was concentrated in vacuo. The residue was purified using silica gel column chromatography eluting with EA in PE [Gradient: 0-50%] to afford the compound 7-({1-tert-butyl-3-[(3R)-3-[(tert-butyldimethylsilyl)oxy]cyclopentyl]-1H-pyrazol-5-yl}amino)-2-[(4-methoxyphenyl)methyl]-3,4-dihydro-2H-5,1λ\hat{ }6,2-benzoxathiazepine-1,1-dione (900 mg, 1.37 mmol, 84.2%) as a brown solid. LCMS: ESI m/z 655 [M+H]⁺

Step E: 7-((1-(tert-butyl)-3-((1S,3R)-3-hydroxycyclopentyl)-1H-pyrazol-5-yl)amino)-2-(4-methoxybenzyl)-3,4-dihydro-2H-benzo[b][1,4,5]oxathiazepine 1,1-dioxide A solution of 7-[(1-tert-butyl-3-{3-[(tert-butyldimethylsilyl)oxy]cyclopentyl}-1H-pyrazol-5-yl)amino]-2-[(4-methoxyphenyl)methyl]-3,4-dihydro-2H-5,1λ^{A}6,2-benzoxathiazepine-1,1-dione (900 mg, 1.37 mmol) in HCOOH (6 mL) was stirred at rt for 1 h. The reaction was concentrated and added MeOH (10 mL) and LiOH (2 mL, 4.12 mmol), and the reaction was stirred at rt for 30 min. The reaction was diluted with H₂O, the aqueous layer was extracted with EA. Combined the EA layer and washed with saturated NaCl solution. The EA layer was dried by Na₂SO₄, filtered and concentrated. The residue was purified using silica gel column chromatography eluting with [gradient: 0-100%] to afford the title compound 7-{[1-tert-butyl-3-(3-hydroxycyclopentyl)-1H-pyrazol-5-yl]amino}-2-[(4-methoxyphenyl)methyl]-3,4-dihydro-2H-5,1λ\hat{ }6,2-benzoxathiazepine-1,1-dione (700 mg, 1.30 mmol, 94.2%) as a white solid.

Step F: (1R,3S)-3-(1-(tert-butyl)-5-((2-(4-methoxybenzyl)-1,1-dioxido-3,4-dihydro-2H-benzo[b][1,4,5]oxathiazepin-7-yl)amino)-1H-pyrazol-3-yl)cyclopentyl (4-nitrophenyl) carbonate To a solution of 7-{[1-tert-butyl-3-(3-hydroxycyclopentyl)-1H-pyrazol-5-yl]amino}-2-[(4-methoxyphenyl)methyl]-3,4-dihydro-2H-5,1λ^{A}6,2-benzoxathiazepine-1,1-dione (400 mg, 0.740 mmol) in THF (5 mL) and DCM (5 mL) were added Py (117 mg, 1.48 mmol), DMAP (0.900 mg, 7 μmol) and 4-nitrophenyl chloroformate (194 mg, 0.962 mmol). The reaction was stirred at rt for 3 h. The reaction was concentrated in vacuo. The residue was purified using silica gel column chromatography eluting with EA in PE [gradient: 0-100%] to afford the title compound 3-[1-tert-butyl-5-({2-[(4-methoxyphenyl)methyl]-1,1-dioxo-3,4-dihydro-2H-5,1λ\hat{ }6,2-benzoxathiazepin-7-yl}amino)-1H-pyrazol-3-yl]cyclopentyl 4-nitrophenyl carbonate (450 mg, 0.638 mmol, 86.2%) as a white solid. LCMS: ESI m/z 706 [M+H]⁺

Step G: (1R,3S)-3-(5-((1,1-dioxido-3,4-dihydro-2H-benzo[b][1,4,5]oxathiazepin-7-yl)amino)-1H-pyrazol-3-yl)cyclopentyl (4-nitrophenyl) carbonate A solution of 7-{[1-tert-butyl-3-(3-hydroxycyclopentyl)-1H-pyrazol-5-yl]amino}-2-[(4-methoxyphenyl)methyl]-3,4-dihydro-2H-5,1λ\hat{ }6,2-benzoxathiazepine-1,1-dione (400 mg, 0.740 mmol) in HCOOH (10 mL) was stirred at 100° C. overnight. The cooled reaction mixture was concentrated in vacuo to afford the title compound 3-{5-[(1,1-dioxo-3,4-dihydro-2H-5,11λ\hat{ }6,2-benzoxathiazepin-7-yl)amino]-1H-pyrazol-3-yl}cyclopentyl 4-nitrophenyl carbonate (250 mg, 0.472 mmol, 83.3%) as a brown solid.

Step H: (1R,3S)-3-(3-((1,1-dioxido-3,4-dihydro-2H-benzo[b][1,4,5]oxathiazepin-7-yl)amino)-1H-pyrazol-5-yl)cyclopentyl isopropylcarbamate A solution of 3-{5-[(1,1-dioxo-3,4-dihydro-2H-5,1λ\hat{ }6,2-benzoxathiazepin-7-yl)amino]-1H-pyrazol-3-yl}cyclopentyl 4-nitrophenyl carbonate (250 mg, 0.472 mmol) in isopropylamine (5 mL) was stirred at 50° C. for 2 h. The reaction concentrated in vacuo. The residue was purified by prep-HPLC to afford the title compound 3-{5-[(1,1-dioxo-3,4-dihydro-2H-5,11λ\hat{ }6,2-benzoxathiazepin-7-yl)amino]-1H-pyrazol-3-yl}cyclopentyl N-(propan-2-yl)carbamate (100 mg, 0.222 mmol, 47.1%) as a white solid. LCMS: ESI m/z 450 [M+H]⁺

¹H NMR (400 MHz, DMSO) δ 11.89 (s, 1H), 8.94 (s, 1H), 7.49 (d, J=8.8 Hz, 1H), 7.35 (s, 1H), 7.23 (s, 1H), 7.02 (d, J=8.8 Hz, 1H), 6.95 (s, 1H), 5.67 (s, 1H), 5.00 (s, 1H), 4.04 (s, 2H), 3.57 (s, 1H), 3.37 (s, 2H), 3.05 (s, 1H), 2.48-2.37 (m, 1H), 2.25-1.80 (m, 2H), 1.71 (s, 2H), 1.60 (s, 1H), 1.03 (d, J=4.8 Hz, 6H).

### Example 7: (1s,4s)-4-{3-[(1,1-dioxo-2,3-dihydro-1λ\hat{ }6, 2-benzothiazol-5-yl)amino]-1H-pyrazol-5-yl}cyclohexyl N-(propan-2-yl)carbamate

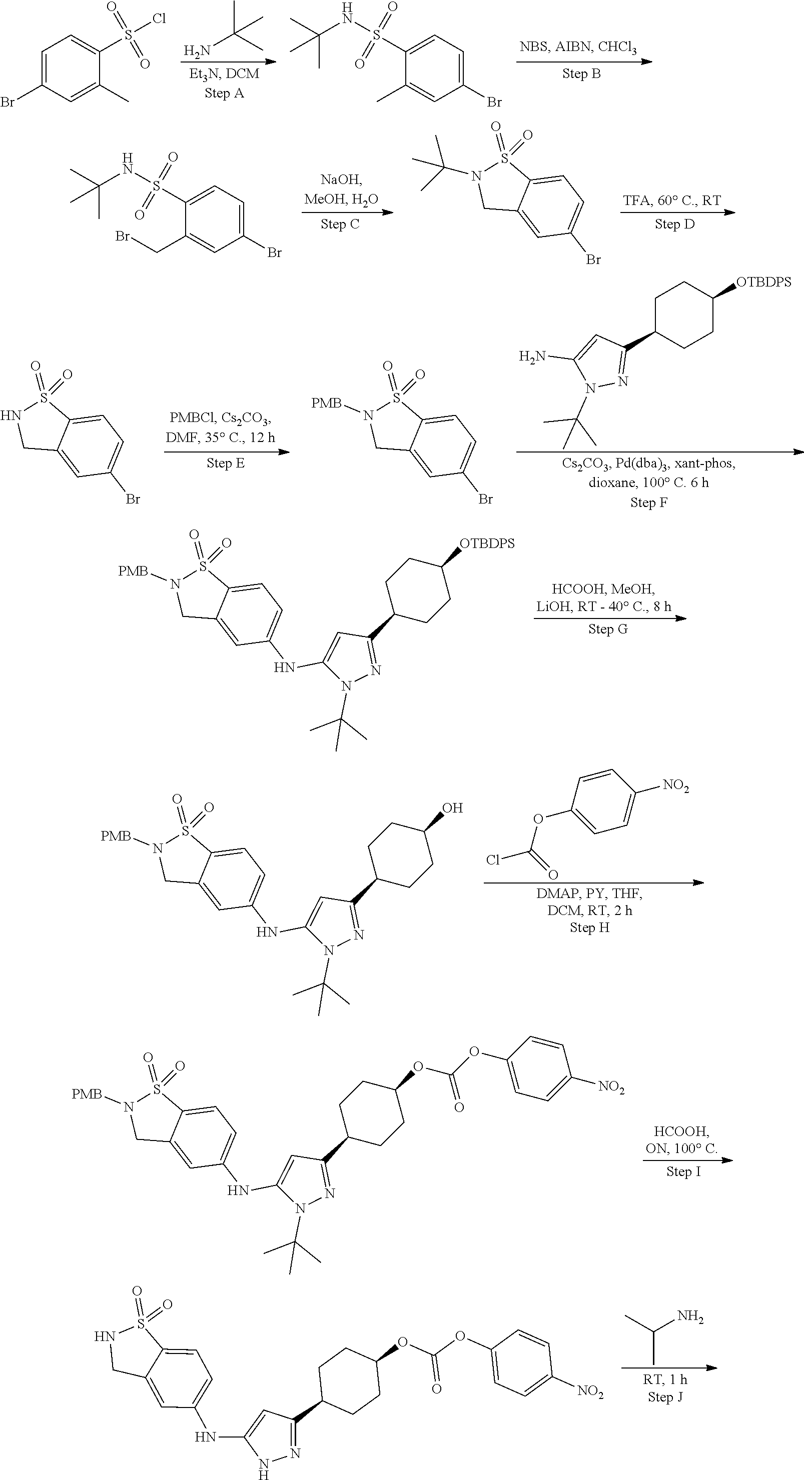

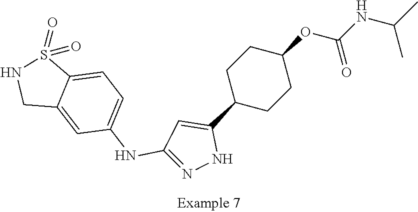 [SMILES: CC(C)NC(=O)O[C@H]1CC[C@@H](c2cc(Nc3ccc4c(c3)CNS4(=O)=O)n[nH]2)CC1]

Step A: 4-bromo-N-(tert-butyl)-2-methylbenzenesulfonamide To a solution of 4-bromo-2-methylbenzene-1-sulfonyl chloride (5 g, 18.5 mmol) in DCM (20 mL) were added triethylamine (7.73 mL, 55.6 mmol), 2-methylpropan-2-amine (3.93 mL, 37.1 mmol). The reaction was stirred at room temperature overnight. The reaction was concentrated in vacuo. The residue was purified using silica gel column chromatography eluting with [gradient: 0-50%] to afford the title compound 4-bromo-N-tert-butyl-2-methylbenzene-1-sulfonamide (5 g, 16.3 mmol, 88.0%) as a white solid. LCMS: ESI m/z 306 [M+H]⁺. ¹H NMR (400 MHz, DMSO) δ 7.75 (d, J=8.4 Hz, 1H), 7.65-7.49 (m, 3H), 2.54 (s, 3H), 1.06 (s, 9H).

Step B: 4-bromo-2-(bromomethyl)-N-(tert-butyl)benzenesulfonamide To a solution of 4-bromo-N-tert-butyl-2-methylbenzene-1-sulfonamide (5 g, 16.3 mmol) in CHCl₃ (10 mL) were added AIBN (268 mg, 1.63 mmol), DIEA (0.448 mL, 2.71 mmol), and NBS (2.91 g, 16.3 mmol). The reaction was stirred at 60° C. overnight. The reaction was concentrated. The residue was purified using silica gel column chromatography eluting with [gradient: 0-30%] to afford the title compound 4-bromo-2-(bromomethyl)-N-tert-butylbenzene-1-sulfonamide (1.9 g, 4.9 mmol, 30.2%) as a white solid. LCMS: ESI m/z 384 [M+H]⁺.

Step C: 5-bromo-2-(tert-butyl)-2,3-dihydrobenzo[d]isothiazole 1,1-dioxide To a solution of 4-bromo-2-(bromomethyl)-N-tert-butylbenzene-1-sulfonamide (1.4 g, 3.63 mmol) in MeOH (20 mL) and H₂O (5 mL) was added NaOH (0.44 g, 10.9 mmol) and the reaction was stirred at room temperature for 3 hr. The reaction was concentrated in vacuo. The residue was purified using silica gel column chromatography eluting with EA in PE [gradient: 0-80%] to afford the title compound 5-bromo-2-tert-butyl-2,3-dihydro-1λ\hat{ }6,2-benzothiazole-1,1-dione (900 mg, 2.95 mmol, 81.4%) as a white solid. LCMS: ESI m/z 304 [M+H]⁺. ¹H NMR (400 MHz, DMSO) δ 7.83 (d, J=0.8 Hz, 1H), 7.85-7.63 (m, 2H), 4.55 (s, 2H), 1.46 (s, 9H).

Step D: 5-bromo-2,3-dihydro-1λ\hat{ }6, 2-benzothiazole-1,1-dione A solution of 5-bromo-2-tert-butyl-2,3-dihydro-1λ\hat{ }6, 2-benzothiazole-1,1-dione (3 g, 9.86 mmol) in TFA (50 mL) was stirred at 60° C. 3 hr. The reaction mixture was concentrated. The residue was adjusted to pH value 7-8 with aq. 1 N NaOH solution (3 mL). The following mixture was diluted with EA and water. The organic layer was separated, washed with brine, dried over Na₂SO₄ and concentrated in vacuo. The residue was purified using silica gel column chromatography eluting with ethyl acetate in petroleum ether (gradient:0-50%) to afford the title compound 5-bromo-2,3-dihydro-1λ\hat{ }6, 2-benzothiazole-1,1-dione (2.1 g, 8.46 mmol, 85.8%) as a white solid. LCMS: ESI m/z 248.10 [M+H]⁺

Step E: 5-bromo-2-[(4-methoxyphenyl)methyl]-2,3-dihydro-1λ\hat{ }6, 2-benzothiazole-1,1-dione To a solution of 5-bromo-2,3-dihydro-1λ\hat{ }6, 2-benzothiazole-1,1-dione (2.1 g, 8.46 mmol) in DMF (20 mL) were added 4-Methoxybenzylchloride (1.99 g, 12.7 mmol) and Cs₂CO₃ (5.52 g, 16.9 mmol). The reaction was stirred at 35° C. overnight. The reaction mixture was diluted with EA and water. The organic layer was separated, washed with brine, dried over Na₂SO₄ and concentrated in vacuo. The residue was purified using silica gel column chromatography eluting with ethyl acetate in petroleum ether (gradient:0-20%) to afford the title compound 5-bromo-2-[(4-methoxyphenyl)methyl]-2,3-dihydro-1λ\hat{ }6, 2-benzothiazole-1,1-dione (2.45 g, 6.65 mmol, 78.6%) as a white solid. LCMS: ESI m/z 368.25 [M+H]⁺

Step F: 5-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-2,3-dihydro-1λ\hat{ }6, 2-benzothiazole-1,1-dione To a solution of 5-bromo-2-[(4-methoxyphenyl)methyl]-2,3-dihydro-1λ\hat{ }6, 2-benzothiazole-1,1-dione (500 mg, 1.36 mmol) in dioxane (10 mL) were added 1-tert-butyl-3-[(1s,4s)-4-[(tert-butyldiphenylsilyl)oxy]cyclohexyl]-1H-pyrazol-5-amine (775 mg, 1.63 mmol), Cs₂CO₃ (1.32 g, 4.07 mmol), Pd₂(dba)₃ (124 mg, 0.136 mmol) and Xant-PHOS (157 mg, 0.272 mmol). The reaction was stirred at 100° C. under N₂ for 6 hr. The reaction was diluted with EA and water. The organic layer was separated, washed with brine, dried over Na₂SO₄ and concentrated in vacuo. The residue was purified using silica gel column chromatography eluted with ethyl acetate in petroleum ether (gradient:0-50%) to afford the title compound 5-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-2,3-dihydro-1λ\hat{ }6, 2-benzothiazole-1,1-dione (700 mg, 0.917 mmol, 67.6%) as a white solid. LCMS: ESI m/z 763.09 [M+H]⁺

Step G: 5-({1-tert-butyl-3-[(1s,4s)-4-hydroxycyclohexyl]-1H-pyrazol-5-yl}amino)-2-[(4-methoxyphenyl)methyl]-2,3-dihydro-1λ\hat{ }6, 2-benzothiazole-1,1-dione To a solution of 5-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-2,3-dihydro-1λ\hat{ }6, 2-benzothiazole-1,1-dione (700 mg, 0.917 mmol) in TBAF (20 mL, 1M in THF) and the reaction was stirred at room temperature overnight. The reaction was diluted with EA and water. The organic layer was separated, washed with saturated NaCl solution and concentrated in vacuo. The residue was purified using silica gel column chromatography eluted with ethyl acetate in petroleum ether (gradient: 0-30%) to afford the title compound 5-({1-tert-butyl-3-[(1s,4s)-4-hydroxycyclohexyl]-1H-pyrazol-5-yl}amino)-2-[(4-methoxyphenyl)methyl]-2,3-dihydro-1λ\hat{ }6,2-benzothiazole-1,1-dione (400 mg, 0.762 mmol, 83.1%) as a white solid. LCMS: ESI m/z 524.69 [M+H]⁺

Step H: 4-nitrophenyl (1s,4s)-4-[1-tert-butyl-5-({2-[(4-methoxyphenyl)methyl]-1,1-dioxo-2,3-dihydro-1λ\hat{ }6, 2-benzothiazol-5-yl}amino)-1H-pyrazol-3-yl]cyclohexyl carbonate To a solution of 5-({1-tert-butyl-3-[(1s,4s)-4-hydroxycyclohexyl]-1H-pyrazol-5-yl}amino)-2-[(4-methoxyphenyl)methyl]-2,3-dihydro-1λ\hat{ }6, 2-benzothiazole-1,1-dione (400 mg, 0.762 mmol) in THF (5 mL) and DCM (5 mL) were added 4-nitrophenyl chloroformate (230 mg, 1.14 mmol), DMAP (9.31 mg, 0.076 mmol) and Py (0.123 mL, 1.53 mmol). The reaction was stirred at room temperature under N₂ for 3 hr. The reaction was diluted with EA and water. The organic layer was separated, washed with brine, dried over Na₂SO₄ and concentrated in vacuo. The residue was purified using silica gel column chromatography elutied with ethyl acetate in petroleum ether (gradient:0-50%) to afford the title compound 4-nitrophenyl (1s,4s)-4-[1-tert-butyl-5-({2-[(4-methoxyphenyl)methyl]-1,1-dioxo-2,3-dihydro-1λ\hat{ }6, 2-benzothiazol-5-yl}amino)-1H-pyrazol-3-yl]cyclohexyl carbonate (250 mg, 0.362 mmol, 47.5%) as a white solid. LCMS: ESI m/z 689.79 [M+H]⁺

Step I: 4-nitrophenyl (1s,4s)-4-{5-[(1,1-dioxo-2,3-dihydro-1λ\hat{ }6, 2-benzothiazol-5-yl)amino]-1H-pyrazol-3-yl}cyclohexyl carbonate A solution of 4-nitrophenyl (1s,4s)-4-[1-tert-butyl-5-({2-[(4-methoxyphenyl)methyl]-1,1-dioxo-2,3-dihydro-1λ\hat{ }6, 2-benzothiazol-5-yl}amino)-1H-pyrazol-3-yl]cyclohexyl carbonate (70 mg, 0.101 mmol) in formic acid (5 mL) was stirred at 100° C. overnight. The reaction was concentrated in vacuo to afford the title compound 4-nitrophenyl (1s,4s)-4-{5-[(1,1-dioxo-2,3-dihydro-1λ\hat{ }6, 2-benzothiazol-5-yl)amino]-1H-pyrazol-3-yl}cyclohexyl carbonate (50 mg, 0.097 mmol, 96.0%) as a brown solid. LCMS: ESI m/z 513.53 [M+H]⁺

Step J: (1s,4s)-4-{3-[(1,1-dioxo-2,3-dihydro-1λ\hat{ }6, 2-benzothiazol-5-yl)amino]-1H-pyrazol-5-yl}cyclohexyl N-(propan-2-yl)carbamate To a solution of 4-nitrophenyl (1s,4s)-4-{5-[(1,1-dioxo-2,3-dihydro-1λ\hat{ }6,2-benzothiazol-5-yl)amino]-1H-pyrazol-3-yl}cyclohexyl carbonate (50 mg, 0.097 mmol) in isopropylamine (3 mL). The reaction was stirred at room temperature for 1 hr. The reaction was purified by prep-HPLC (C18, 0-50% acetonitrile in H₂O) to afford the title compound (1s,4s)-4-{3-[(1,1-dioxo-2,3-dihydro-1λ\hat{ }6, 2-benzothiazol-5-yl)amino]-1H-pyrazol-5-yl}cyclohexyl N-(propan-2-yl)carbamate (14.5 mg, 0.033 mmol, 34.4%) as a white solid. LCMS: ESI m/z 433.53 [M+H]⁺. ¹H NMR (400 MHz, DMSO) δ 11.89 (s, 1H), 9.01 (s, 1H), 7.57-7.26 (m, 4H), 6.88 (s, 1H), 5.68 (s, 1H), 4.75 (s, 1H), 4.29 (s, 2H), 3.60 (s, 1H), 2.68 (s, 1H), 1.85-1.59 (m, 8H), 1.05 (d, J=5.2 Hz, 6H).

### Example 8: (1R,3S)-3-(3-((1-oxoisoindolin-5-yl)amino)-1H-pyrazol-5-yl)cyclopentyl isopropylcarbamate

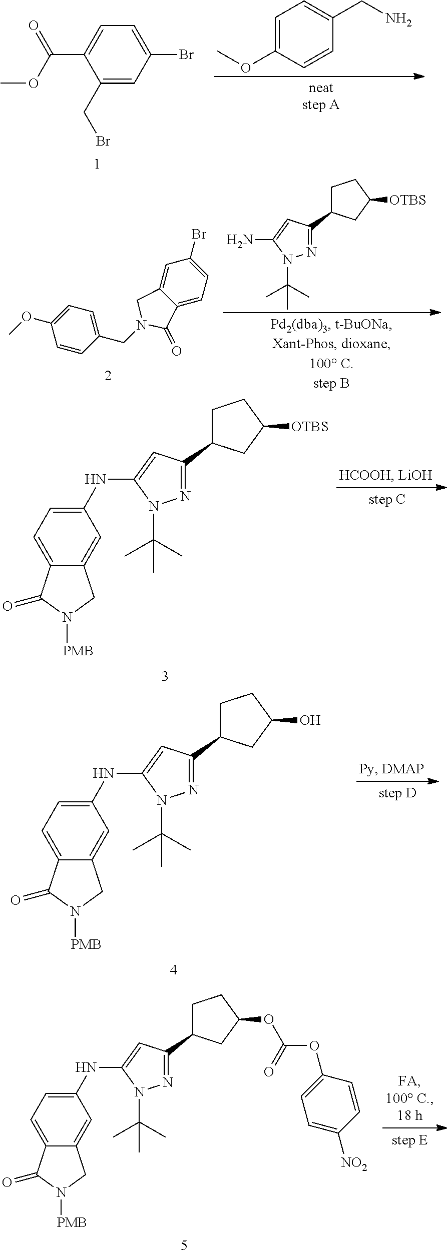

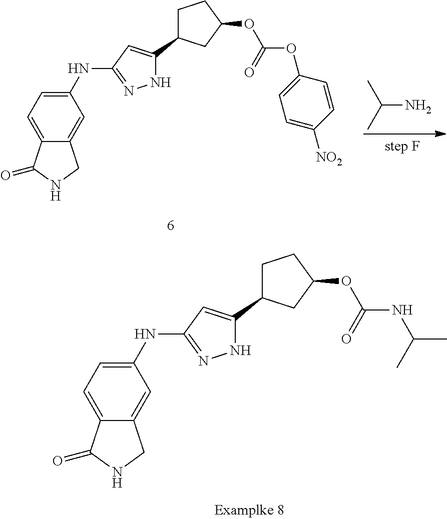

Step A: 5-bromo-2-(4-methoxybenzyl)isoindolin-1-one To a solution of methyl 4-bromo-2-(bromomethyl)benzoate (4.50 g, 14.6 mmol) in tetrahydrofuran (100 mL) was added (4-methoxyphenyl)methanamine (2.20 g, 16.0 mmol). The mixture was stirred at 80° C. for 2 h. The cooled mixture was concentrated under vacuum. The residue was purified by flash column chromatography with 0-50% ethyl acetate in petroleum ether to afford 5-bromo-2-[(4-methoxyphenyl)methyl]-2,3-dihydro-1H-isoindol-1-one (2 g, 6.02 mmol, 41.2%) as a white solid. LCMS: ESI 332.4, 334.4 [M+H]⁺. ¹H NMR (400 MHz, methyl sulfoxide-d₆) δ7.81 (s, 1H), 7.72-7.61 (m, 2H), 7.25-7.17 (m, 2H), 6.95-6.86 (m, 2H), 4.64 (s, 2H), 4.32 (s, 2H), 3.73 (s, 3H).

Step B: 5-((1-(tert-butyl)-3-((1S,3R)-3-((tert-butyldimethylsilyl)oxy)cyclopentyl)-1H-pyrazol-5-yl)amino)-2-(4-methoxybenzyl)isoindolin-1-one To a solution of 1-tert-butyl-3-[(1S,3R)-3-[(tert-butyldimethylsilyl)oxy]cyclopentyl]-1H-pyrazol-5-amine (500 mg, 1.48 mmol) in dioxane (5 mL) were added 5-bromo-2-[(4-methoxyphenyl)methyl]-2,3-dihydro-1H-isoindol-1-one (737 mg, 2.22 mmol), [5-(diphenylphosphanyl)-9,9-dimethyl-9H-xanthen-4-yl]diphenylphosphane (171 mg, 0.296 mmol), sodium 2-methylpropan-2-olate (2.96 mL, 2.96 mmol) and Pd₂(dba)₃ (135 mg, 0.148 mmol). the reaction was stirred at 90° C. under N₂ for 18 hr. The reaction was diluted with EA and water. The organic layer was separated, washed with further brine, and concentrated in vacuo. The residue was purified using silica gel column chromatography eluting with ethyl acetate in petroleum ether (3/1) The organic layer was collected, concentrated in vacuo, and dried to afford the title compound 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-1H-isoindol-1-one (500 mg, 57% yield) as a yellow solid. LCMS: ESI m/z 589 [M+H]⁺

Step C: 5-((1-(tert-butyl)-3-((1S,3R)-3-hydroxycyclopentyl)-1H-pyrazol-5-yl)amino)-2-(4-methoxybenzyl)isoindolin-1-one To a solution of 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-1H-isoindol-1-one (500 mg, 0.85 mmol) in HCOOH (5 mL) and the reaction was stirred at room temperature for 1 hr. The reaction was concentrated and were added MeOH (5 mL), and LiOH (142 mg, 3.39 mmol), and the reaction was stirred at room temperature for 30 min. The reaction was diluted with water The aqueous layer was extracted with EA. Combined the EA layer and washed with saturated NaCl solution. The EA layer was dried by Na₂SO₄, filtered and concentrated. The residue was purified using silica gel column chromatography eluting with [gradient: 0-100%] to afford the title compound 5-({1-tert-butyl-3-[(1S,3R)-3-hydroxycyclopentyl]-1H-pyrazol-5-yl}amino)-2-[(4-methoxyphenyl)methyl]-2,3-dihydro-1H-isoindol-1-one (300 mg, 74% yield) as a yellow oil. LCMS: ESI m/z 475 [M+H]⁺

Step D: (3S,5S)-1-acetyl-5-(1-(tert-butyl)-5-(3-(methoxymethyl)-1-methyl-1H-pyrazole-5-carboxamido)-1H-pyrazol-3-yl)pyrrolidin-3-yl (4-nitrophenyl) carbonate To a solution of 5-({1-tert-butyl-3-[(1S,3R)-3-hydroxycyclopentyl]-1H-pyrazol-5-yl}amino)-2-[(4-methoxyphenyl)methyl]-2,3-dihydro-1H-isoindol-1-one (300 mg, 0.632 mmol) in THF (10 mL) and DCM (10 mL) were added 4-nitrophenyl chloroformate (191 mg, 0.948 mmol), pyridine (0.153 mL, 1.896 mmol). The reaction was stirred at 30° C. for 30 min. The reaction was concentrated in vacuo. The residue was purified using silica gel column chromatography eluting with EA in PE [gradient: 0-100%] to afford the title compound (1R,3S)-3-[1-tert-butyl-5-({2-[(4-methoxyphenyl)methyl]-1-oxo-2,3-dihydro-1H-isoindol-5-yl}amino)-1H-pyrazol-3-yl]cyclopentyl 4-nitrophenyl carbonate (300 mg, 75% yield) as a white solid. LCMS: ESI m/z 640 [M+H]⁺

Step E: 4-nitrophenyl ((1R,3S)-3-(3-((1-oxoisoindolin-5-yl)amino)-1H-pyrazol-5-yl)cyclopentyl) carbonate A solution of (1R,3S)-3-[1-tert-butyl-5-({2-[(4-methoxyphenyl)methyl]-1-oxo-2,3-dihydro-1H-isoindol-5-yl}amino)-1H-pyrazol-3-yl]cyclopentyl 4-nitrophenyl carbonate (300 mg, 0.469 mmol) in HCOOH (5 mL) was stirred at 100° C. overnight. The reaction was concentrated in vacuo to afford the title compound 4-nitrophenyl (1R,3S)-3-{5-[(1-oxo-2,3-dihydro-1H-isoindol-5-yl)amino]-1H-pyrazol-3-yl}cyclopentyl carbonate (300 mg, crude) as a white solid, which was used into next step without further purification. LCMS: ESI m/z 464 [M+H]⁺

Step F: (1R,3S)-3-(3-((1-oxoisoindolin-5-yl)amino)-1H-pyrazol-5-yl)cyclopentyl isopropylcarbamate To a solution of 4-nitrophenyl (1R,3S)-3-{5-[(1-oxo-2,3-dihydro-1H-isoindol-5-yl)amino]-1H-pyrazol-3-yl}cyclopentyl carbonate (300 mg, 0.647 mmol) in THF (2 mL) was added propan-2-amine (0.555 mL, 6.47 mmol). The reaction was stirred at 50° C. for 2 hr. The reaction concentrated in vacuo. The residue was purified by prep-HPLC (0.1% TFA) to afford the title compound (1R,3S)-3-{5-[(1-oxo-2,3-dihydro-1H-isoindol-5-yl)amino]-1H-pyrazol-3-yl}cyclopentyl N-(propan-2-yl)carbamate (4.8 mg, 1.9% yield) as a white solid. LCMS: ESI m/z 384 [M+H]⁺. ¹H NMR (400 MHz, DMSO-d₆) δ 8.78 (brs, 1H), 8.03 (s, 1H), 7.54 (s, 1H), 7.43 (d, J=8.4 Hz, 1H), 7.24 (d, J=8.4 Hz, 1H), 6.94 (d, J=7.6 Hz, 1H), 5.70 (s, 1H), 5.00 (s, 1H), 4.26 (s, 2H), 3.11-3.03 (m, 1H), 2.11-1.52 (m, 6H), 1.03 (d, J=6.4 Hz, 6H).

### Example 9: (1R,3S)-3-(3-((1,1-dioxido-2,3-dihydrobenzo[b]thiophen-5-yl)amino)-1H-pyrazol-5-yl)cyclopentyl isopropylcarbamate

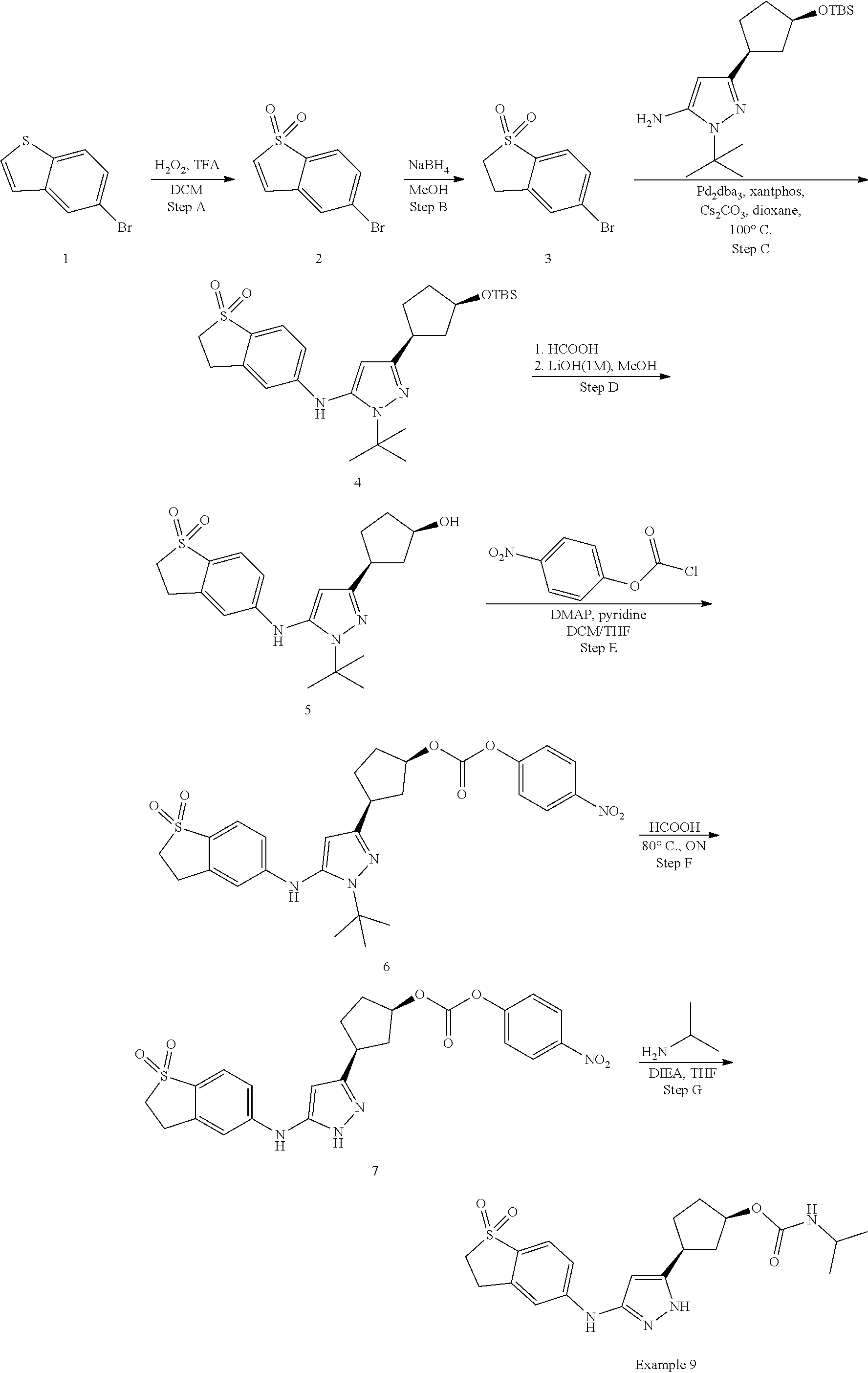

Step A: 5-bromo-1λ\hat{ }6-benzothiophene-1,1-dione To a solution of 5-bromo-1-benzothiophene (1 g, 4.69 mmol) in DCM (8 mL) was added H₂O₂(2 mL, 19.6 mmol, 30% in H₂O) and TFA (2 mL, 26.92 mmol). The mixture was stirred at 20° C. for 5 hours. The reaction mixture was diluted with water, extracted with DCM (30 mL×3). The organic phase was washed with aqueous Na₂S₂O₃, dried over Na₂SO₄ and concentrated. The residue was purified by silica gel chromatography (10 g column) using 0-30% EtOAc/hexane to afford 5-bromo-1λ\hat{ }6-benzothiophene-1,1-dione (700 mg, 2.85 mmol, 60.8%) as a brown oil.

Step B: 5-bromo-2,3-dihydro-1λ\hat{ }6-benzothiophene-1,1-dione To a solution of 5-bromo-1λ\hat{ }6-benzothiophene-1,1-dione (700 mg, 2.85 mmol) in MeOH (30 mL) was added NaBH₄ (221 mg, 5.71 mmol) and the mixture was stirred at 20° C. for 2 hours. H₂O (0.5 mL) was added to quench. The following mixture was concentrated and the residue was purified by silica gel chromatography (10 g column) using 0-30% EtOAc/hexane to afford 5-bromo-2,3-dihydro-1λ\hat{ }6-benzothiophene-1,1-dione (540 mg, 2.18 mmol, 76.5%) as a brown oil. ¹H NMR (400 MHz, DMSO) δ 7.84 (s, 1H), 7.72 (s, 2H), 3.66-3.54 (m, 2H), 3.42-3.28 (m, 2H).

Step C: 5-({1-tert-butyl-3-[(1S,3R)-3-[(tert-butyldimethylsilyl)oxy]cyclopentyl]-1H-pyrazol-5-yl}amino)-2,3-dihydro-1λ\hat{ }6-benzothiophene-1,1-dione To a solution of 5-bromo-2,3-dihydro-1λ\hat{ }6-benzothiophene-1,1-dione (100 mg, 0.40 mmol) and 1-tert-butyl-3-[(1S,3R)-3-[(tert-butyldimethylsilyl)oxy]cyclopentyl]-1H-pyrazol-5-amine (150 mg, 0.44 mmol) in dioxane (5 mL) was added Cs₂CO₃ (263 mg, 0.81 mmol), Pd₂(dba)₃ (37.0 mg, 0.04 mmol) and Xant-PHOS (23.4 mg, 0.04 mmol). The reaction mixture was stirred at 100° C. under N₂ for 5 hours. The mixture was purified by silica gel chromatography (2 g column) using 0-50% EtOAc/hexane. The product-containing fractions were concentrated to afford 5-({1-tert-butyl-3-[(1S,3R)-3-[(tert-butyldimethylsilyl)oxy] cyclopentyl]-1H-pyrazol-5-yl}amino)-2,3-dihydro-1λ\hat{ }6-benzothiophene-1,1-dione (160 mg, 0.32 mmol, 78.5%) as a brown solid. LCMS: ESI m/z 504.3 [M+H]⁺

Step D: 5-({1-tert-butyl-3-[(1S,3R)-3-hydroxycyclopentyl]-1H-pyrazol-5-yl}amino)-2,3-dihydro-1λ\hat{ }6-benzothiophene-1,1-dione A solution of 5-({1-tert-butyl-3-[(1S,3R)-3-[(tert-butyldimethylsilyl)oxy]cyclopentyl]-1H-pyrazol-5-yl}amino)-2,3-dihydro-1λ\hat{ }6-benzothiophene-1,1-dione (160 mg, 0.32 mmol) in formic acid (4 mL) was stirred at 20° C. for 30 min. The mixture was concentrated under vacuum to remove formic acid to give a crude, which was added MeOH (3 mL) and LiOH (100 mg) was stirred at 20° C. for 30 min. The mixture was concentrated under vacuum and poured into EA (30 mL), extracted with water. The organic layer was concentrated to give 5-({1-tert-butyl-3-[(1S,3R)-3-hydroxycyclopentyl]-1H-pyrazol-5-yl}amino)-2,3-dihydro-1λ\hat{ }6-benzothiophene-1,1-dione (120 mg, 0.31 mmol, 97.0%) as a brown solid. LCMS: ESI m/z 390.1 [M+H]⁺

Step E: (1R,3S)-3-{1-tert-butyl-5-[(1,1-dioxo-2,3-dihydro-1λ\hat{ }6-benzothiophen-5-yl)amino]-1H-pyrazol-3-yl}cyclopentyl 4-nitrobenzoate To a solution of 5-({1-tert-butyl-3-[(1S,3R)-3-hydroxycyclopentyl]-1H-pyrazol-5-yl}amino)-2,3-dihydro-1λ\hat{ }6-benzothiophene-1,1-dione (120 mg, 0.31 mmol) in DCM (2 mL) and THF (2 mL) was added 4-nitrophenyl chloroformate (93.14 mg, 0.46 mmol), pyridine (0.05 mL, 0.62 mmol) and DMAP (3.76 mg, 0.03 mmol). The mixture was stirred at 20° C. for 1 hours. The mixture was concentrated and the residue was purified by silica gel chromatography (2 g column) using 0-50% EtOAc/hexane to afford (1R,3S)-3-{1-tert-butyl-5-[(1,1-dioxo-2,3-dihydro-1λ\hat{ }6-benzothiophen-5-yl)amino]-1H-pyrazol-3-yl}cyclopentyl 4-nitrobenzoate (130 mg, 0.24 mmol, 78.3%) as a brown solid. LCMS: ESI m/z 555.2 [M+H]⁺

Step F: (1R,3S)-3-{5-[(1,1-dioxo-2,3-dihydro-1λ\hat{ }6-benzothiophen-5-yl)amino]-1H-pyrazol-3-yl}cyclopentyl 4-nitrophenyl carbonate A solution of (1R,3S)-3-{1-tert-butyl-5-[(1,1-dioxo-2,3-dihydro-1λ\hat{ }6-benzothiophen-5-yl)amino]-1H-pyrazol-3-yl}cyclopentyl 4-nitrophenyl carbonate (130 mg, 0.23 mmol) in formic acid (4 mL) was stirred at 100° C. for 12 hours. The cooled mixture was concentrated under vacuum to give (1R,3S)-3-{5-[(1,1-dioxo-2,3-dihydro-1λ\hat{ }6-benzothiophen-5-yl)amino]-1H-pyrazol-3-yl}cyclopentyl 4-nitrophenyl carbonate (110 mg, 0.2 mmol, 84.7%) as a brown solid. The crude product was used for the next step without purification. LCMS: ESI m/z 499.1 [M+H]⁺

Step G: (1R,3S)-3-(3-((1,1-dioxido-2,3-dihydrobenzo[b]thiophen-5-yl)amino)-1H-pyrazol-5-yl)cyclopentyl isopropylcarbamate To a solution of (1R,3S)-3-{5-[(1,1-dioxo-2,3-dihydro-1λ\hat{ }6-benzothiophen-5-yl)amino]-1H-pyrazol-3-yl}cyclopentyl 4-nitrophenyl carbonate (110 mg, 0.2 mmol) in THF (5 mL) was added ethyldiisopropylamine (0.11 mL, 0.66 mmol). Then propan-2-amine (0.09 mL, 1.10 mmol) was added dropwise. The reaction mixture was stirred at 20° C. for 2 hours. The mixture was concentrated and the residue was purified by prep-TLC (EA) to give (1R,3S)-3-(3-((1,1-dioxido-2,3-dihydrobenzo[b]thiophen-5-yl)amino)-1H-pyrazol-5-yl)cyclopentyl isopropylcarbamate (14.7 mg, 0.035 mmol, 16%) as a white solid. LCMS: ESI m/z 419.1 [M+H]⁺. ¹H NMR (400 MHz, DMSO) δ 11.92 (s, 1H), 8.99 (s, 1H), 7.52-7.40 (m, 2H), 7.29 (d, J=8.4 Hz, 1H), 6.95 (d, J=7.2 Hz, 1H), 5.71 (s, 1H), 5.00 (brs, 1H), 3.64-3.52 (m, 1H), 3.48 (t, J=6.8 Hz, 2H), 3.24 (t, J=6.8 Hz, 2H), 3.12-3.01 (m, 1H), 2.49-2.42 (m, 1H), 2.03 (dd, J=15.6, 7.6 Hz, 1H), 1.94-1.82 (m, 1H), 1.78-1.64 (m, 2H), 1.63-1.52 (m, 1H), 1.04 (d, J=6.4 Hz, 6H).

### Example 10: (1R,3S)-3-(5-((3-methyl-1,1-dioxido-2,3-dihydrobenzo[d]isothiazol-5-yl)amino)-1H-pyrazol-3-yl)cyclopentyl isopropylcarbamate

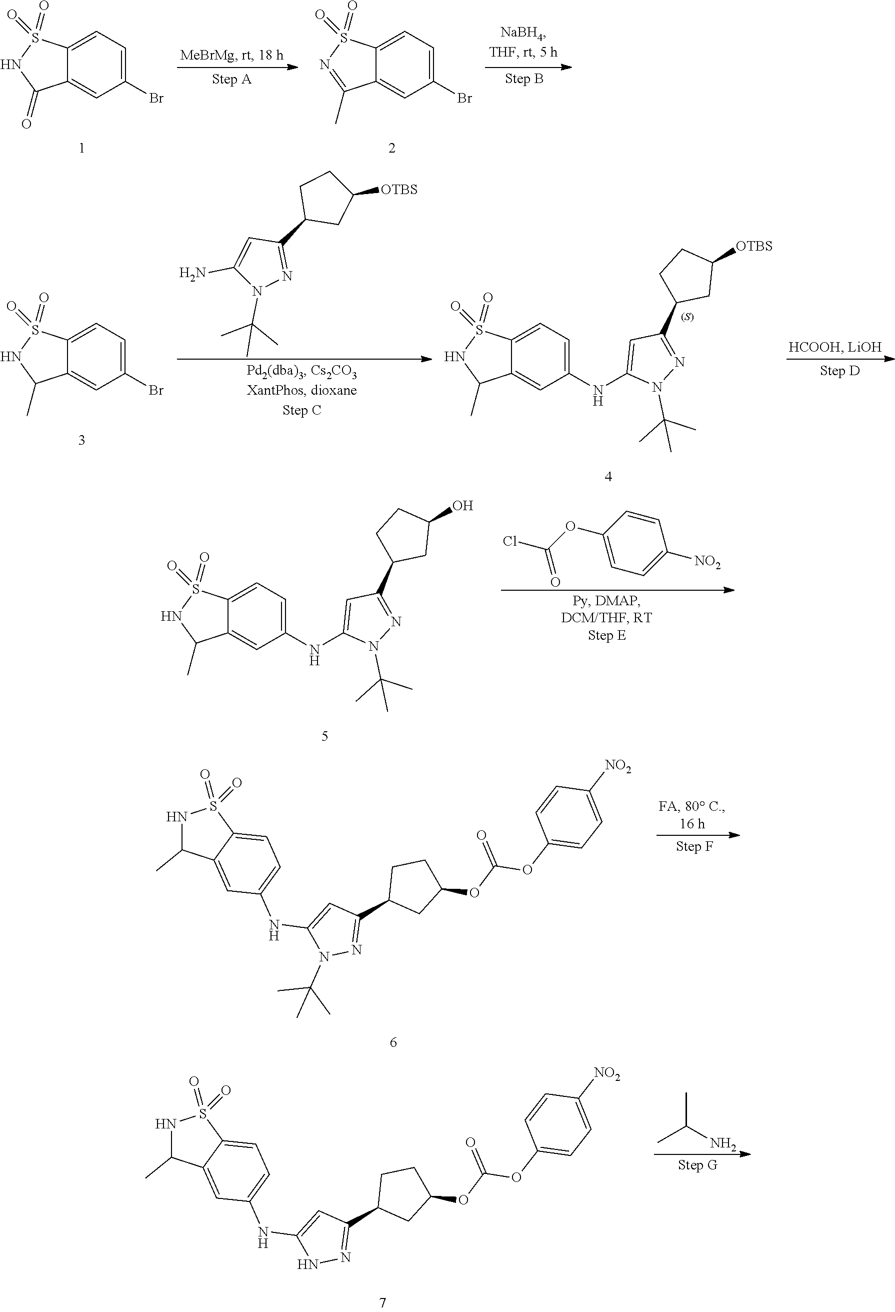

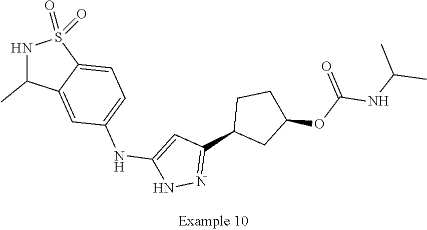 [SMILES: CC(C)NC(=O)O[C@@H]1CC[C@H](c2cc(Nc3ccc4c(c3)C(C)NS4(=O)=O)[nH]n2)C1]

Step A: 5-bromo-3-methylbenzo[d]isothiazole 1,1-dioxide A solution of 5-bromo-2,3-dihydro-1λ\hat{ }6,2-benzothiazole-1,1,3-trione (500 mg, 1.91 mmol) in THF (0.1 M) was treated with MeMgBr (5.7 mL, 5.72 mmol, 1M in THF) at 0° C. The mixture was stirred at rt overnight, then quenched with water and extracted with EtOAc (100 mL). The combined organic layers were dried over anhydrous Na₂SO₄, filtered, and concentrated under reduced pressure. The residue was purified by flash chromatography on silica gel (PE/EA=2:1) to afford pure product 5-bromo-3-methyl-1λ\hat{ }6,2-benzothiazole-1,1-dione (400 mg, 1.54 mmol, 80.6%) as a white solid. LCMS: ESI 259.9, 261.9 [M+H]⁺

Step B: 5-bromo-3-methyl-2,3-dihydrobenzo[d]isothiazole 1,1-dioxide To a solution of 5-bromo-3-methyl-1λ\hat{ }6,2-benzothiazole-1,1-dione (400 mg, 1.54 mmol) in THF (20 mL) was added NaBH₄ (104 mg, 3.07 mmol), after that the reaction mixture was stirred at 25° C. for 5 h. After completion, the reaction mixture was quenched by NH₄Cl (aq.) 2 mL. The reaction was diluted with EA and water. The organic layer was separated, washed with further brine, and concentrated in vacuo. The residue was purified using silica gel column chromatography eluting with PE/EA=1:3. The organic layer was collected, concentrated in vacuo, and dried to afford the title compound 5-bromo-3-methyl-2,3-dihydro-1λ10 b 6,2-benzothiazole-1,1-dione (350 mg, 1.33 mmol, 86.8%) as a colorless oil. LCMS: ESI m/z 261.9, 263.9 [M+H]⁺

Step C: 5-((1-(tert-butyl)-3-((1S,3R)-3-((tert-butyldimethylsilyl)oxy)cyclopentyl)-1H-pyrazol-5-yl)amino)-3-methyl-2,3-dihydrobenzo[d]isothiazole 1,1-dioxide To a solution of 1-tert-butyl-3-[(1S,3R)-3-[(tert-butyldimethylsilyl)oxy]cyclopentyl]-1H-pyrazol-5-amine (350 mg, 1.04 mmol) in dioxane (2 mL) were added 5-bromo-3-methyl-2,3-dihydro-1λ\hat{ }6,2-benzothiazole-1,1-dione (300 mg, 1.14 mmol), [5-(diphenylphosphanyl)-9,9-dimethyl-9H-xanthen-4-yl]diphenylphosphane (120 mg, 0.207 mmol), Cs₂CO₃ (675 mg, 2.074 mmol) and Pd₂(dba)₃ (95 mg, 0.104 mmol). The reaction mixture was stirred at 90° C. for 18 hr. The reaction was diluted with EA and water. The organic layer was separated, washed with further brine, and concentrated in vacuo. The residue was purified using silica gel column chromatography eluting with ethyl acetate in petroleum ether (3/1) afford the title compound 5-({1-tert-butyl-3-[(1S,3R)-3-[(tert-butyldimethylsilyl)oxy]cyclopentyl]-1H-pyrazol-5-yl}amino)-3-methyl-2,3-dihydro-1λ\hat{ }6,2-benzothiazole-1,1-dione (200 mg, 0.386 mmol, 37.2%) as a yellow solid. LCMS: ESI m/z 519.2 [M+H]⁺

Step D: 5-((1-(tert-butyl)-3-((1S,3R)-3-hydroxycyclopentyl)-1H-pyrazol-5-yl)amino)-3-methyl-2,3-dihydrobenzo[d]isothiazole 1,1-dioxide A solution of 5-({1-tert-butyl-3-[(1S,3R)-3-[(tert-butyldimethylsilyl)oxy]cyclopentyl]-1H-pyrazol-5-yl}amino)-3-methyl-2,3-dihydro-1λ\hat{ }6,2-benzothiazole-1,1-dione (200 mg, 0.386 mmol) in HCOOH (50 mL) was stirred at room temperature for 1 hr. The reaction was concentrated and were added MeOH (50 mL), and LiOH (64.7 mg, 1.54 mmol), and the reaction was stirred at room temperature for 30 min. The reaction was diluted with H₂O. The following mixture was extracted with EA. Combined the EA layer and washed with saturated NaCl solution. The EA layer was dried by Na₂SO₄, filtered and concentrated. The residue was purified using silica gel column chromatography eluting with [gradient: 80-100%] to afford the title compound 5-({1-tert-butyl-3-[(1S,3R)-3-hydroxycyclopentyl]-1H-pyrazol-5-yl}amino)-3-methyl-2,3-dihydro-1λ\hat{ }6,2-benzothiazole-1,1-dione (120 mg, 0.297 mmol, 76.9%) as a white solid. LCMS: ESI m/z 405 [M+H]⁺

Step E: (1R,3S)-3-(1-(tert-butyl)-5-((3-methyl-1,1-dioxido-2,3-dihydrobenzo[d]isothiazol-5-yl)amino)-1H-pyrazol-3-yl)cyclopentyl (4-nitrophenyl) carbonate To a solution of 5-({1-tert-butyl-3-[(1S,3R)-3-hydroxycyclopentyl]-1H-pyrazol-5-yl}amino)-3-methyl-2,3-dihydro-11λ\hat{ }6,2-benzothiazole-1,1-dione (120 mg, 0.297 mmol) in THF (10 mL) and DCM (10 mL) were added 4-nitrophenyl chloroformate (89.7 mg, 0.445 mmol), pyridine (0.072 mL, 0.890 mmol). The reaction was stirred at 50° C. for 30 min. The reaction was concentrated in vacuo. The residue was purified using silica gel column chromatography eluting with EA in PE [gradient: 70-90%] to afford the title compound (1R,3S)-3-{1-tert-butyl-5-[(3-methyl-1,1-dioxo-2,3-dihydro-1λ\hat{ }6,2-benzothiazol-5-yl)amino]-1H-pyrazol-3-yl}cyclopentyl 4-nitrophenyl carbonate (100 mg, 0.176 mmol, 59.2%) as a white solid. LCMS: ESI m/z 570.1 [M+H]⁺

Step F: (1R,3S)-3-(5-((3-methyl-1,1-dioxido-2,3-dihydrobenzo[d]isothiazol-5-yl)amino)-1H-pyrazol-3-yl)cyclopentyl (4-nitrophenyl) carbonate A solution of (1R,3S)-3-{1-tert-butyl-5-[(3-methyl-1,1-dioxo-2,3-dihydro-1λ\hat{ }6,2-benzothiazol-5-yl)amino]-1H-pyrazol-3-yl}cyclopentyl 4-nitrophenyl carbonate (100 mg, 0.176 mmol) in HCOOH (5 mL) was stirred at 80° C. overnight. The reaction was concentrated in vacuo to afford the title compound (1R,3S)-3-{5-[(3-methyl-1,1-dioxo-2,3-dihydro-1λ\hat{ }6,2-benzothiazol-5-yl)amino]-1H-pyrazol-3-yl}cyclopentyl 4-nitrophenyl carbonate (80 mg, 0.156 mmol, 88.9%) as a white solid, which was used into next step without further purification. LCMS: ESI m/z 514 [M+H]⁺.

Step G: (1R,3S)-3-(5-((3-methyl-1,1-dioxido-2,3-dihydrobenzo[d]isothiazol-5-yl)amino)-1H-pyrazol-3-yl)cyclopentyl isopropylcarbamate To a solution of (1R,3S)-3-{5-[(3-methyl-1,1-dioxo-2,3-dihydro-1λ\hat{ }6,2-benzothiazol-5-yl)amino]-1H-pyrazol-3-yl}cyclopentyl 4-nitrophenyl carbonate (80 mg, 0.156 mmol) in THF (2 mL) was added propan-2-amine (0.133 mL, 1.558 mmol) and the reaction was stirred at 50° C. for 2 hr. The reaction concentrated in vacuo. The residue was purified by prep-HPLC (FA condition) to afford the title compound (1R,3S)-3-{5-[(3-methyl-1,1-dioxo-2,3-dihydro-1λ\hat{ }6,2-benzothiazol-5-yl)amino]-1H-pyrazol-3-yl}cyclopentyl N-(propan-2-yl)carbamate (2.4 mg, 3.55%) as a white solid. LCMS: ESI m/z 434 [M+H]⁺. ¹H NMR (400 MHz, DMSO-d₆) δ 11.92 (s, 1H), 9.01 (s, 1H), 7.57 (d, J=4.4 Hz, 1H), 7.50 (d, J=8.8 Hz, 2H), 7.32 (d, J=8.8 Hz, 1H), 6.94 (d, J=7.6 Hz, 1H), 5.69 (s, 1H), 5.06-4.94 (m, 1H), 4.67-4.54 (m, 1H), 3.65-3.53 (m, 1H), 3.13-2.98 (m, 1H), 2.48-2.40 (m, 1H), 2.06-1.97 (m, 1H), 1.95-1.82 (m, 1H), 1.79-1.53 (m, 3H), 1.39 (d, J=6.4 Hz, 3H), 1.03 (d, J=8 Hz, 6H).

### Example 11: (1s,4s)-4-{3-[(1,1-dioxo-3,4-dihydro-2H-1λ\hat{ }6,2-benzothiazin-6-yl)amino]-1H-pyrazol-5-yl}cyclohexyl N-(propan-2-yl)carbamate

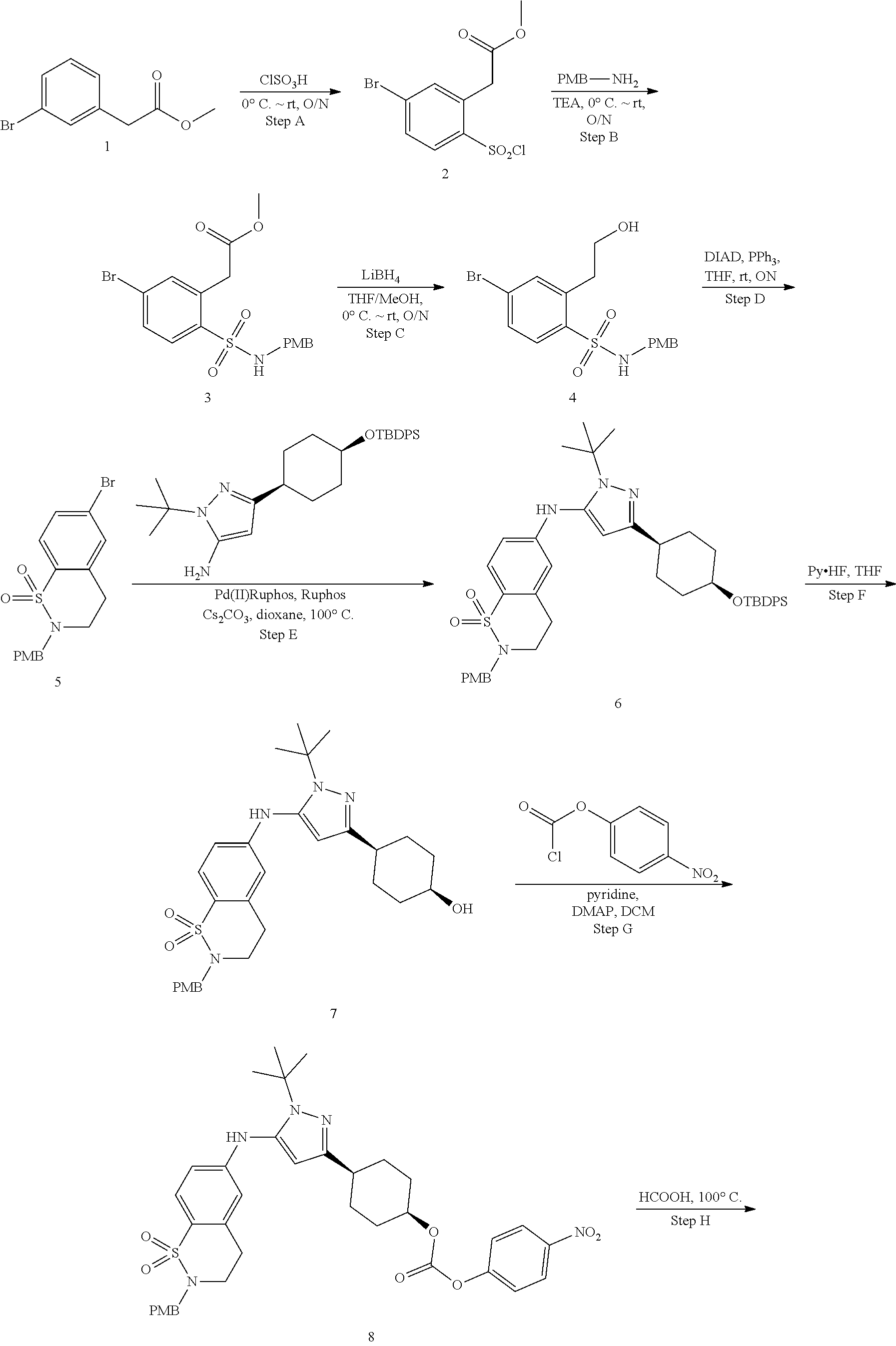

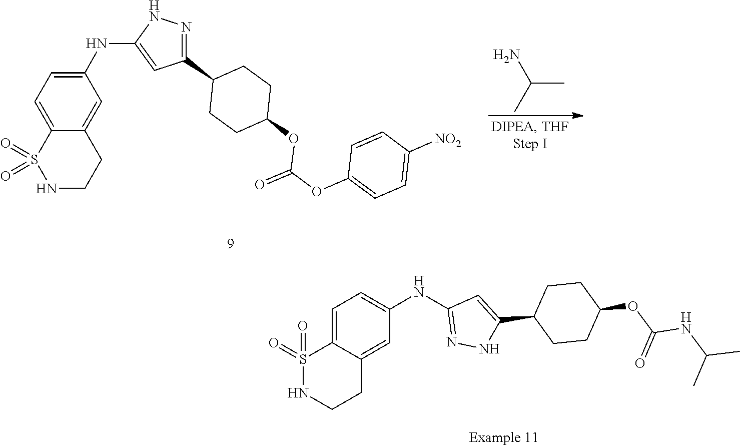

Step A. methyl 2-(5-bromo-2-(chlorosulfonyl)phenyl)acetate A reaction mixture of methyl 2-(3-bromophenyl)acetate (10 g, 43.8 mmol) in HSO₃Cl (5.10 g, 43.8 mmol) was stirred at rt overnight. The reaction mixture was poured into ice-water slowly and extracted with EtOAc. The combined organic phase was washed with brine, dried over Na₂SO₄, filtered and concentrated to give crude methyl 2-[5-bromo-2-(chlorosulfonyl)phenyl]acetate (3 g, 9.2 mmol, 21%) as a yellow oil. LCMS: m/z 327 [M+H]⁺.

Step B. methyl 2-(5-bromo-2-(N-(4-methoxybenzyl)sulfamoyl)phenyl)acetate To a stirred mixture of methyl 2-[5-bromo-2-(chlorosulfonyl)phenyl]acetate (3 g, 9.2 mmol) in DCM (30 mL) was added (4-methoxyphenyl)methanamine (1.26 g, 9.2 mmol) and TEA (1.86 g, 18.4 mmol) slowly at 0° C. After stirred at rt overnight, the mixture was poured into water and extracted with DCM. The combined organic phase was washed with brine, dried with Na₂SO₄, filtered and concentrated. The residue was purified by chromatography (silica gel, 0-35%, EtOAc in PE) to give methyl 2-(5-bromo-2-{[(4-methoxyphenyl)methyl]sulfamoyl}phenyl)acetate (2 g, 4.65 mmol, 33.9%) as a brown solid. LCMS: m/z 429 [M+H]⁺.

Step C. 4-bromo-2-(2-hydroxyethyl)-N-(4-methoxybenzyl)benzenesulfonamide To a stirred mixture of methyl 2-(5-bromo-2-{[(4-methoxyphenyl)methyl]sulfamoyl}phenyl)acetate (2.5 g, 5.84 mmol) in THF (30 mL) was added lithium(1+) ion boranuide (5.84 mL, 11.6 mmol) slowly at 0° C. After stirred at rt overnight, the mixture was poured into ice-water and extracted with EtOAc. The combined organic phase was washed with brine, dried with Na₂SO₄, filtered and concentrated in vacuum and the residue was purified by flash chromatography to afford 4-bromo-2-(2-hydroxyethyl)-N-[(4-methoxyphenyl)methyl]benzene-1-sulfonamide (1.5 g, 3.75 mmol, 64.2%) as a colorless oil. LCMS: m/z 401 [M+H]⁺.

Step D. 6-bromo-2-(4-methoxybenzyl)-3,4-dihydro-2H-benzo[e][1,2]thiazine 1,1-dioxide To a stirred mixture of 4-bromo-2-(2-hydroxyethyl)-N-[(4-methoxyphenyl)methyl]benzene-1-sulfonamide (700 mg, 1.749 mmol) in THF (10 mL) was added triphenylphosphane (917 mg, 3.49 mmol) and (E)-N-{[(propan-2-yloxy)carbonyl]imino}(propan-2-yloxy)formamide (0.69 mL, 3.49 mmol) at rt. After stirred at rt overnight, the mixture was poured into water and extracted with EtOAc. The combined organic phase was washed with brine, dried with Na₂SO₄, filtered and concentrated. The residue was purified by chromatography to give 6-bromo-2-[(4-methoxyphenyl)methyl]-3,4-dihydro-2H-1λ\hat{ }6,2-benzothiazine-1,1-dione (300 mg, 0.785 mmol, 44.9%) as a white solid. LCMS: m/z 383 [M+H]⁺.

Step E. 6-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-3,4-dihydro-2H-1λ\hat{ }6,2-benzothiazine-1,1-dione To a stirred mixture of 6-bromo-2-[(4-methoxyphenyl)methyl]-3,4-dihydro-2H-1λ\hat{ }6,2-benzothiazine-1,1-dione (600 mg, 1.570 mmol) in dioxane (10 mL) was added 1-tert-butyl-3-[(1s,4s)-4-[(tert-butyldiphenylsilyl)oxy]cyclohexyl]-1H-pyrazol-5-amine (746 mg, 1.570 mmol), Cs₂CO₃ (1.28 g, 3.92 mmol), RuPhos (146 mg, 0.314 mmol) and 2-(2-aminophenyl)benzen-1-ide; chloropalladiumylium; {2-[2,6-bis(propan-2-yloxy)phenyl]phenyl}dicyclohexylphosphane (122 mg, 0.157 mmol) at rt. After stirred at 100° C. overnight, the mixture was poured into water and extracted with EtOAc. The combined organic phase was washed with brine, dried with Na₂SO₄, filtered and concentrated. The residue was purified by chromatography to give 6-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-3,4-dihydro-2H-1λ\hat{ }6,2-benzothiazine-1,1-dione (600 mg, 0.772 mmol, 49.2%) as a colorless oil. LCMS: m/z 777 [M+H]⁺.

Step F. 6-({1-tert-butyl-3-[(1s,4s)-4-hydroxycyclohexyl]-1H-pyrazol-5-yl}amino)-2-[(4-methoxyphenyl)methyl]-3,4-dihydro-2H-1λ\hat{ }6,2-benzothiazine-1,1-dione A mixture of 6-({1-tert-butyl-3-[(1s,4s)-4-[(tert-butyldiphenylsilyl)oxy]cyclohexyl]-1H-pyrazol-5-yl}amino)-2-[(4-methoxyphenyl)methyl]-3,4-dihydro-2H-1λ\hat{ }6,2-benzothiazine-1,1-dione (450 mg, 0.579 mmol) in THF (10 mL) was added pyridine hydrofluoride (5 mL). The reaction mixture was stirred at 25° C. overnight. The mixture was adjusted pH to 8 by NaHCO₃(aqueous), extracted with. The organic layer was separated, washed with brine, dried over Na₂SO₄, filtered and concentrated in vacuo. The residue was purified by silica gel column chromatography eluted with ethyl acetate in petroleum ether to afford the title compound 6-({1-tert-butyl-3-[(1s,4s)-4-hydroxycyclohexyl]-1H-pyrazol-5-yl}amino)-2-[(4-methoxyphenyl)methyl]-3,4-dihydro-2H-1λ\hat{ }6,2-benzothiazine-1,1-dione (130 mg, 0.241 mmol, 41.6%) as a colorless oil. LCMS: m/z 539 [M+H]⁺.

Step G. 4-nitrophenyl (1s,4s)-4-[1-tert-butyl-5-({2-[(4-methoxyphenyl)methyl]-1,1-dioxo-3,4-dihydro-2H-1λ\hat{ }6, 2-benzothiazin-6-yl]cyclohexyl carbonate To a stirred solution of 6-({1-tert-butyl-3-[(1s,4s)-4-hydroxycyclohexyl]-1H-pyrazol-5-yl}amino)-2-[(4-methoxyphenyl)methyl]-3,4-dihydro-2H-1λ\hat{ }6,2-benzothiazine-1,1-dione (130 mg, 0.241 mmol) in DCM (2 mL)/THF (2 mL) was added 4-nitrophenyl chloroformate (145 mg, 0.724 mmol), Py (0.039 mL, 0.483 mmol) and DMAP (2.95 mg, 0.024 mmol) at rt. After stirred at rt for 3 h, the mixture was poured into water (30 ml) and extracted with EtOAc. The combined organic phase was washed with brine, dried over Na₂SO₄, filtered and concentrated. The residue was purified by chromatography (silica gel, 0-10%, MeOH in DCM) to give 4-nitrophenyl (1s,4s)-4-[1-tert-butyl-5-({2-[(4-methoxyphenyl)methyl]-1,1-dioxo-3,4-dihydro-2H-1λ\hat{ }6,2-benzothiazin-6-yl}amino)-1H-pyrazol-3-yl]cyclohexyl carbonate (70 mg, 0.099 mmol, 41.2%) as a brown solid. LCMS: m/z 704 [M+H]⁺.

Step H. 4-nitrophenyl (1s,4s)-4-{5-[(1,1-dioxo-3,4-dihydro-2H-1λ\hat{ }6, 2-benzothiazin-6-yl)amino]-1H-pyrazol-3-yl}cyclohexyl carbonate A mixture of 4-nitrophenyl (1s,4s)-4-[1-tert-butyl-5-({2-[(4-methoxyphenyl)methyl]-1,1-dioxo-3,4-dihydro-2H-1λ\hat{ }6,2-benzothiazin-6-yl}amino)-1H-pyrazol-3-yl]cyclohexyl carbonate (70 mg, 0.099 mmol) in formic acid (3 mL) was stirred at 100° C. overnight, then concentrated under reduced pressure to give crude 4-nitrophenyl (1s,4s)-4-{5-[(1,1-dioxo-3,4-dihydro-2H-1λ\hat{ }6,2-benzothiazin-6-yl)amino]-1H-pyrazol-3-yl}cyclohexyl carbonate (60 mg, 0.097 mmol, 97.2%) as a brown oil. LCMS: m/z 528 [M+H]⁺.

Step I. (1s,4s)-4-{3-[(1,1-dioxo-3,4-dihydro-2H-1λ\hat{ }6, 2-benzothiazin-6-yl)amino]-1H-pyrazol-5-yl}cyclohexyl N-(propan-2-yl)carbamate To a stirred solution of 4-nitrophenyl (1s,4s)-4-{5-[(1,1-dioxo-3,4-dihydro-2H-1λ\hat{ }6,2-benzothiazin-6-yl)amino]-1H-pyrazol-3-yl}cyclohexyl carbonate (60 mg, 0.114 mmol) in THF (3 mL) was added propan-2-amine (0.019 mL, 0.227 mmol). The reaction mixture was stirred at room temperature for 2 h. The reaction was concentrated. The residue was purified by prep-HPLC to give (1s,4s)-4-{3-[(1,1-dioxo-3,4-dihydro-2H-1λ\hat{ }6,2-benzothiazin-6-yl)amino]-1H-pyrazol-5-yl}cyclohexyl N-(propan-2-yl)carbamate (46.4 mg, 0.104 mmol, 91.1%) as a brown solid. LCMS: m/z 448 [M+H]⁺. ¹H NMR (400 MHz, DMSO) δ 11.86 (s, 1H), 8.80 (s, 1H), 7.47 (d, J=8.8 Hz, 1H), 7.34-7.19 (m, 2H), 7.14 (s, 1H), 6.88 (d, J=7.2 Hz, 1H), 5.64 (s, 1H), 4.75 (s, 1H), 3.65-3.55 (m, 1H), 3.50 (dd, J=12.8, 6.4 Hz, 2H), 2.83-2.77 (m, 2H), 2.73-2.65 (m, 1H), 1.82-1.75 (m, 4H), 1.75-1.62 (m, 4H), 1.05 (d, J=6.4 Hz, 6H).

### Example 12: (1R,3S)-3-{3-[(4-methyl-1,1-dioxo-3,4-dihydro-2H-1λ\hat{ }6,2,4-benzothiadiazin-6-yl)amino]-1H-pyrazol-5-yl}cyclopentyl N-(propan-2-yl)carbamate

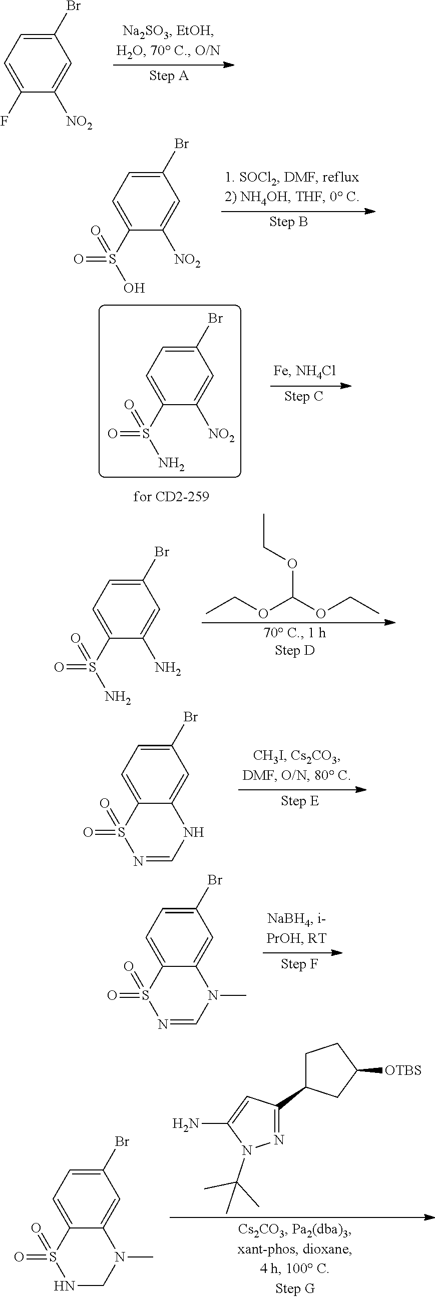

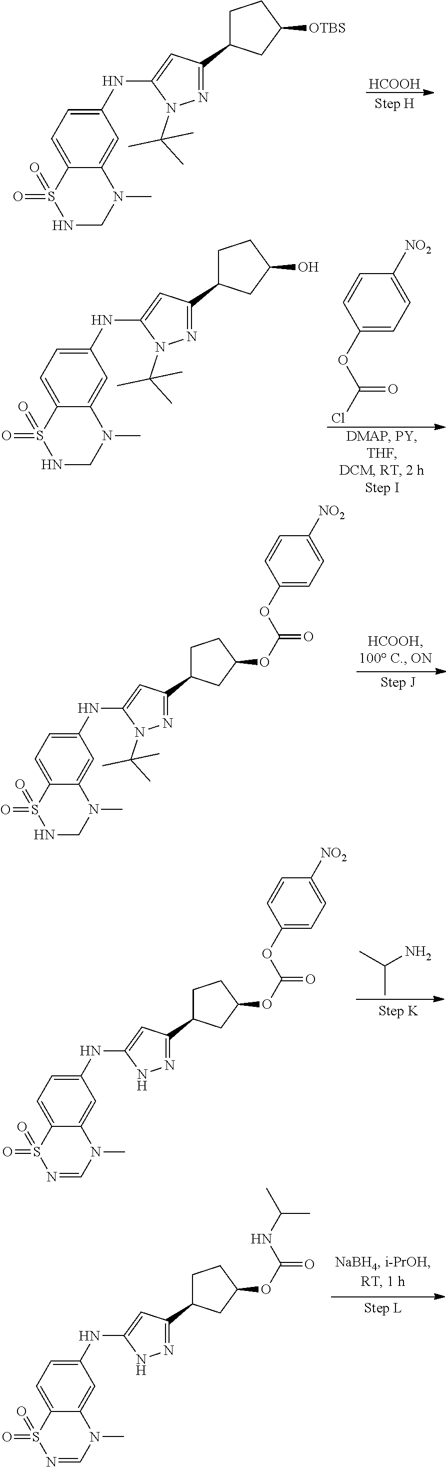

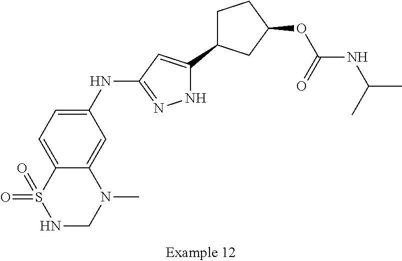 [SMILES: CC(C)NC(=O)O[C@@H]1CC[C@H](c2cc(Nc3ccc4c(c3)N(C)CNS4(=O)=O)n[nH]2)C1]

Step A: 4-bromo-2-nitrobenzene-1-sulfonic acid To a solution of 4-bromo-1-fluoro-2-nitrobenzene (2.80 mL, 22.7 mmol) in EtOH (60 mL) was added a suspension of Na₂SO₃ (2.72 mL, 56.8 mmol) in EtOH (100 mL) and H₂O (125 mL) dropwise. The reaction was stirred at 70° C. overnight. The cooled reaction mixture was acidified with HCl (2N) to PH 2. The reaction was diluted with EA and water. The organic layer was separated, washed with saturated NaCl solution, and concentrated in vacuo to afford the title compound 4-bromo-2-nitrobenzene-1-sulfonic acid (12.4 g, 44.0 mmol, 100%) as a white solid. LCMS: ESI m/z 282.07 [M+H]⁺.

Step B: 4-bromo-2-nitrobenzene-1-sulfonamide To a solution of 4-bromo-2-nitrobenzene-1-sulfonic acid (10 g, 35.5 mmol) in SOCl₂ (20 mL) was added DMF (0.5 mL). The reaction was stirred at 90° C. for 2 hr. The cooled reaction mixture was concentrated in vacuo. The residue was azeotroped with chloroform. The residue is dissolved in tetrahydrofuran. The following solution was added dropwise into NH₄0H (40 mL) at 0° C. The reaction mixture was stirred at rt for 1 h and then was acidified with HCl (2N) to PH 7. The following mixture was diluted with EA and water. The organic layer was separated, washed with brine and concentrated in vacuo to afford the title compound 4-bromo-2-nitrobenzene-1-sulfonamide (4.57 g, 16.3 mmol, 45.9%) as a white solid. LCMS: ESI m/z 281.09 [M+H]⁺

Step C: 2-amino-4-bromobenzene-1-sulfonamide To a solution of 4-bromo-2-nitrobenzene-1-sulfonamide (4.57 g, 16.3 mmol) in EtOH (10 mL) and H₂O (10 mL) were added NH₄Cl (4.35 g, 81.3 mmol) and Fe (4.54 g, 81.3 mmol). The reaction was stirred at 70° C. for 2 h. The cooled reaction mixture was filtered. The filtrate was concentrated. The residue was purified using silica gel column chromatography eluted with methanol in dichloroform (gradient:0-10%) to afford the title compound 2-amino-4-bromobenzene-1-sulfonamide (3.02 g, 12.0 mmol, 74.0%) as a white solid. LCMS: ESI m/z 251.0 [M+H]⁺

Step D: 6-bromo-4H-1λ\hat{ }6,2,4-benzothiadiazine-1,1-dione A reaction mixture of 2-amino-4-bromobenzene-1-sulfonamide (400 mg, 1.593 mmol) in (diethoxymethoxy)ethane (10 mL) was stirred at 70° C. for 1 hr. The reaction mixture was cooled to rt and then filtered. The filter cake was washed with MTBE and dried in vacuo to afford the title compound 6-bromo-4H-1λ\hat{ }6,2,4-benzothiadiazine-1,1-dione (350 mg, 1.34 mmol, 84.2%) as a white solid. LCMS: ESI m/z 261.10 [M+H]⁺

Step E: 6-bromo-4-methyl-4H-1λ\hat{ }6,2,4-benzothiadiazine-1,1-dione To a solution of 6-bromo-4H-1λ\hat{ }6,2,4-benzothiadiazine-1,1-dione (1.2 g, 4.60 mmol) in DMF (5 mL) were added CH₃I (0.98 g, 6.89 mmol) and Cs₂CO₃ (2.99 g, 9.19 mmol). The reaction was stirred at 80° C. overnight. The reaction was diluted with EA and water. The organic layer was separated, washed with brine and concentrated in vacuo. The residue was purified using silica gel column chromatography eluted with ethyl acetate in petroleum ether (gradient: 0-80%) to afford the title compound 6-bromo-4-methyl-4H-1λ\hat{ }6,2,4-benzothiadiazine-1,1-dione (370 mg, 1.35 mmol, 29.3%) as a white solid. LCMS: ESI m/z 275.13 [M+H]⁺

Step F: 6-bromo-4-methyl-3,4-dihydro-2H-1λ\hat{ }6,2,4-benzothiadiazine-1,1-dione To a solution of 6-bromo-4-methyl-4H-1λ\hat{ }6,2,4-benzothiadiazine-1,1-dione (370 mg, 1.35 mmol) in isopropyl alcohol (20 mL) was added NaBH₄ (182 mg, 5.38 mmol). The reaction was stirred at room temperature overnight. The reaction was diluted with EA and water. The organic layer was separated, washed with brine and concentrated in vacuo. The residue was purified using silica gel column chromatography eluted with ethyl acetate in petroleum ether (gradient:0-50%) to afford the title compound 6-bromo-4-methyl-3,4-dihydro-2H-11λ\hat{ }6,2,4-benzothiadiazine-1,1-dione (340 mg, 1.27 mmol, 91.2%) as a white solid. LCMS: ESI m/z 277.14 [M+H]⁺

Step G: 6-({1-tert-butyl-3-[(1S,3R)-3-[(tert-butyldimethylsilyl)oxy]cyclopentyl]-1H-pyrazol-5-yl}amino)-4-methyl-3,4-dihydro-2H-1λ\hat{ }6,2,4-benzothiadiazine-1,1-dione To a solution of 6-bromo-4-methyl-3,4-dihydro-2H-1λ\hat{ }6,2,4-benzothiadiazine-1,1-dione (340 mg, 1.23 mmol) in dioxane (10 mL) were added 1-tert-butyl-3-[(1S,3R)-3-[(tert-butyldimethylsilyl)oxy]cyclopentyl]-1H-pyrazol-5-amine (414 mg, 1.23 mmol), Cs₂CO₃ (1.20 g, 3.68 mmol), Pd₂(dba)₃ (112 mg, 0.123 mmol) and Xant-PHOS (142 mg, 0.245 mmol). The reaction was stirred at 100° C. under N₂ for 3 h. The cooled reaction mixture was diluted with EA and water. The organic layer was separated, washed with brine and concentrated in vacuo. The residue was purified using silica gel column chromatography eluted with methanol in DCM (gradient: 0-10%) to afford the title compound 6-({1-tert-butyl-3-[(1S,3R)-3-[(tert-butyldimethylsilyl)oxy]cyclopentyl]-1H-pyrazol-5-yl}amino)-4-methyl-3,4-dihydro-2H-1λ\hat{ }6,2,4-benzothiadiazine-1,1-dione (320 mg, 0.599 mmol, 48.9%) as a yellow oil. LCMS: ESI m/z 533.82 [M+H]⁺

Step H: 6-({1-tert-butyl-3-[(1S,3R)-3-hydroxycyclopentyl]-1H-pyrazol-5-yl}amino)-4-methyl-3,4-dihydro-2H-1λ\hat{ }6,2,4-benzothiadiazine-1,1-dione A solution of 6-({1-tert-butyl-3-[(1S,3R)-3-[(tert-butyldimethylsilyl)oxy]cyclopentyl]-1H-pyrazol-5-yl}amino)-4-methyl-3,4-dihydro-2H-1λ\hat{ }6,2,4-benzothiadiazine-1,1-dione (320 mg, 0.599 mmol) in formic acid (5 mL) was stirred at room temperature for 1 hr. Then concentrated in vacuo. The residue was dissolved with methanol and adjusted to pH value 12-13 with LiOH (2M in H₂O). The reaction was diluted with EA and water. The organic layer was separated, washed with brine and concentrated in vacuo. The residue was purified using silica gel column chromatography eluted with methanol in dichloroform (gradient:0-10%) to afford the title compound 6-({1-tert-butyl-3-[(1S,3R)-3-hydroxycyclopentyl]-1H-pyrazol-5-yl}amino)-4-methyl-3,4-dihydro-2H-1λ\hat{ }-1λ\hat{ }6,2,4-benzothiadiazine-1,1-dione (90 mg, 0.215 mmol, 35.8%) as a yellow oil. LCMS: ESI m/z 419.55 [M+H]⁺

Step I: (1R,3S)-3-{1-tert-butyl-5-[(4-methyl-1,1-dioxo-3,4-dihydro-2H-1λ\hat{ }6,2,4-benzothiadiazin-6-yl)amino]-1H-pyrazol-3-yl}cyclopentyl 4-nitrophenyl carbonate To a solution of 6-({1-tert-butyl-3-[(1S,3R)-3-hydroxycyclopentyl]-1H-pyrazol-5-yl}amino)-4-methyl-3,4-dihydro-2H-1λ\hat{ }6,2,4-benzothiadiazine-1,1-dione (90 mg, 0.215 mmol) in THF (5 mL) and DCM (5 mL) were added 4-nitrophenyl chloroformate (64.7 mg, 0.322 mmol), DMAP (2.62 mg, 0.021 mmol) and Py (33.9 mg, 0.429 mmol). The reaction was stirred at room temperature for 3 hr. The reaction was diluted with EA and water. The organic layer was separated, washed with saturated NaCl solution and concentrated in vacuo. The residue was purified using silica gel column chromatography eluted with ethyl acetate in petroleum ether (gradient:0-50%) to afford the title compound (1R,3S)-3-{1-tert-butyl-5-[(4-methyl-1,1-dioxo-3,4-dihydro-2H-1λ\hat{ }6,2,4-benzothiadiazin-6-yl)amino]-1H-pyrazol-3-yl}cyclopentyl 4-nitrophenyl carbonate (40 mg, 0.068 mmol, 31.9%) as a brown solid. LCMS: ESI m/z 584.65 [M+H]⁺

Step J: (1R,3S)-3-{5-[(4-methyl-1,1-dioxo-3,4-dihydro-2H-1λ\hat{ }6,2,4-benzothiadiazin-6-yl)amino]-1H-pyrazol-3-yl}cyclopentyl 4-nitrophenyl carbonate To a solution of (1R,3S)-3-{1-tert-butyl-5-[(4-methyl-1,1-dioxo-3,4-dihydro-2H-1λ\hat{ }6,2,4-benzothiadiazin-6-yl)amino]-1H-pyrazol-3-yl}cyclopentyl 4-nitrophenyl carbonate (40 mg, 0.068 mmol) in formic acid (5 mL). The reaction was stirred at 100° C. for overnight. The reaction was concentrated in vacuo to afford the title compound (1R,3S)-3-{5-[(4-methyl-1,1-dioxo-3,4-dihydro-2H-1λ\hat{ }6,2,4-benzothiadiazin-6-yl)amino]-1H-pyrazol-3-yl}cyclopentyl 4-nitrophenyl carbonate (30 mg, 0.057 mmol, 82.9%) as a brown solid. LCMS: ESI m/z 528.55 [M+H]⁺

Step K: (1R,3S)-3-{5-[(4-methyl-1,1-dioxo-4H-1λ\hat{ }6,2,4-benzothiadiazin-6-yl)amino]-1H-pyrazol-3-yl}cyclopentyl N-(propan-2-yl)carbamate To a solution of (1R,3S)-3-{5-[(4-methyl-1,1-dioxo-4H-1λ\hat{ }-1λ\hat{ }6,2,4-benzothiadiazin-6-yl)amino]-1H-pyrazol-3-yl}cyclopentyl 4-nitrophenyl carbonate (40 mg, 0.076 mmol) in propan-2-amine (5 mL, 58.4 mmol). The reaction was stirred at room temperature for 1 h. The reaction was concentrated in vacuo to afford the title compound (1R,3S)-3-{5-[(4-methyl-1,1-dioxo-4H-11λ\hat{ }6,2,4-benzothiadiazin-6-yl)amino]-1H-pyrazol-3-yl}cyclopentyl N-(propan-2-yl)carbamate (30 mg, 0.067 mmol, 88.4%) as a yellow oil. LCMS: ESI m/z 446.53 [M+H]⁺

Step L: (1R,3S)-3-{3-[(4-methyl-1,1-dioxo-3,4-dihydro-2H-1λ\hat{ }6,2,4-benzothiadiazin-6-yl)amino]-1H-pyrazol-5-yl}cyclopentyl N-(propan-2-yl)carbamate To a solution of (1R,3S)-3-{5-[(4-methyl-1,1-dioxo-4H-1λ\hat{ }6,2,4-benzothiadiazin-6-yl)amino]-1H-pyrazol-3-yl}cyclopentyl N-(propan-2-yl)carbamate (20 mg, 0.045 mmol) in Isopropyl alcohol (5 mL) was added NaBH₄ (6.06 mg, 0.179 mmol). The reaction was stirred at room temperature for 1 hr. The reaction was concentrated in vacuo. The residue was purified by prep-HPLC (C18, 0-50% acetonitrile in H₂O) to afford the title compound (1R,3S)-3-{3-[(4-methyl-1,1-dioxo-3,4-dihydro-2H-1λ\hat{ }6,2,4-benzothiadiazin-6-yl)amino]-1H-pyrazol-5-yl}cyclopentyl N-(propan-2-yl)carbamate (2.8 mg, 0.006 mmol, 14.0%) as a white solid. LCMS: ESI m/z 448.55 [M+H]⁺.

¹H NMR (400 MHz, DMSO) δ 11.88 (s, 1H), 8.69 (s, 1H), 7.76 (t, J=7.6 Hz, 1H), 7.30 (d, J=8.4 Hz, 1H), 6.95 (s, 2H), 6.64 (d, J=8.4 Hz, 1H), 5.66 (s, 1H), 5.00 (s, 1H), 4.60 (d, J=8.0 Hz, 2H), 3.63-3.53 (m, 1H), 3.11-3.01 (m, 1H), 2.88 (s, 3H), 2.08-1.88 (m, 2H), 1.85-1.66 (m, 2H), 1.65-1.55 (m, 1H), 1.04 (d, J=6.4 Hz, 6H).

### Example 13: (1R,3S)-3-(3-((3,3-dimethyl-1,1-dioxido-2,3-dihydrobenzo[d]isothiazol-5-yl)amino)-1H-pyrazol-5-yl)cyclopentyl isopropylcarbamate

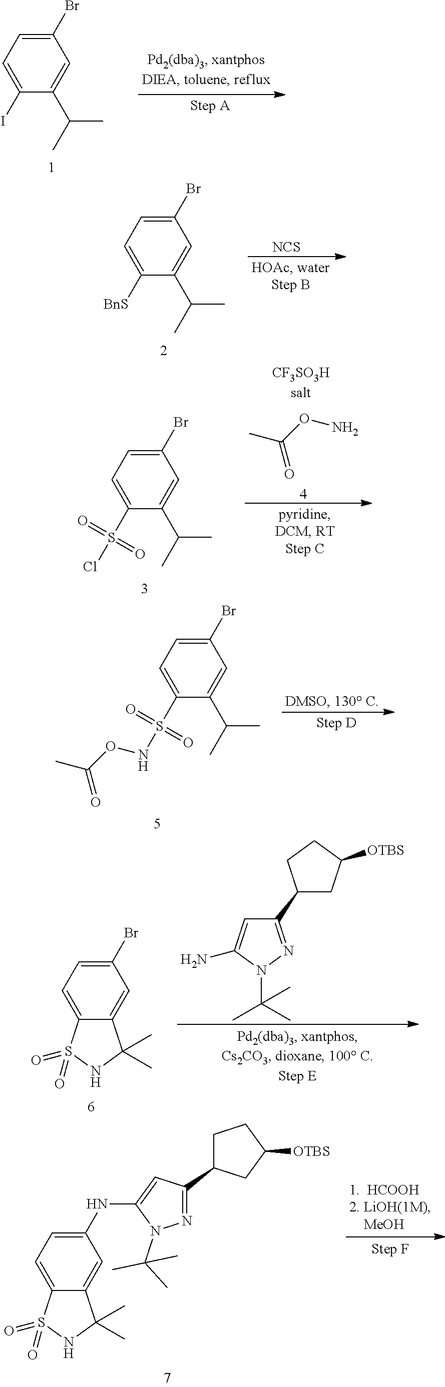

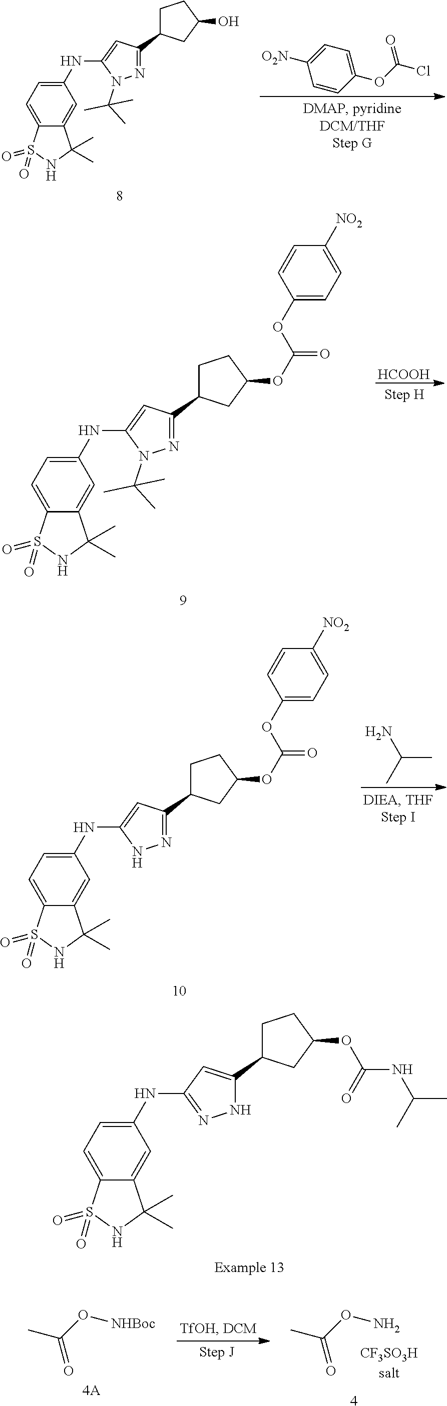

Step A: 1-(benzylsulfanyl)-4-bromo-2-(propan-2-yl)benzene To a solution of 4-bromo-1-iodo-2-(propan-2-yl)benzene (4 g, 12.3 mmol) in toluene (100 mL) was added Xant-PHOS (0.71 g, 1.23 mmol), DIEA EthyldiisopropylaMine (4.06 mL, 24.6 mmol), Pd₂(dba)₃ (1.13 g, 1.23 mmol) and phenylmethanethiol (1.53 g, 1.44 mL, 12.3 mmol). The mixture was stirred at 100° C. under N₂ for 12 hours. The cooled reaction mixture was concentrated and the residue was purified by silica gel chromatography (20 g column) using 0-5% EtOAc/hexane to afford 1-(benzylsulfanyl)-4-bromo-2-(propan-2-yl)benzene (2.66 g, 8.27 mmol, 67.3%) as a brown oil.

Step B: 4-bromo-2-(propan-2-yl)benzene-1-sulfonyl chloride To a solution of 1-(benzylsulfanyl)-4-bromo-2-(propan-2-yl)benzene (2.67 g, 8.31 mmol) in HOAc (15 mL) and water (7.5 mL) was added NCS (3.33 g, 24.9 mmol). The mixture was stirred at 20° C. for 30 min. The reaction mixture was diluted with water 50 mL, extracted with EA (50 mL×3). The organic phase was washed with brine, dried over Na₂SO₄ and concentrated. The residue was purified by silica gel chromatography (15 g column) using 0-5% EtOAc/hexane to afford 4-bromo-2-(propan-2-yl)benzene-1-sulfonyl chloride (2.4 g, 8.06 mmol, 97.0%) as a white solid. ¹H NMR (400 MHz, DMSO) δ 7.90 (d, J=12.0 Hz, 1H), 7.71 (s, 1H), 7.52 (t, J=4.0 Hz, 1H), 4.05-3.98 (m, 1H), 1.35 (d, J=8.0 Hz, 6H).

Step C: 4-bromo-2-(propan-2-yl)benzenesulfonamido acetate To a solution of 4-bromo-2-(propan-2-yl)benzene-1-sulfonyl chloride (2.4 g, 8.06 mmol) in DCM (30 mL) was added pyridine (1.95 mL, 24.1 mmol) and amino acetate (0.61 g, 8.06 mmol). The reaction mixture was stirred at 20° C. for 1 hour. The reaction mixture was diluted with water, extracted with DCM (50 mL×3). The organic phase was washed with brine, dried over Na₂SO₄ and concentrated. The residue was purified by silica gel chromatography (10 g column) using 0-30% EtOAc/hexane to afford 4-bromo-2-(propan-2-yl)benzenesulfonamido acetate (900 mg, 2.67 mmol, 33.2%) as a brown oil. LCMS: ESI m/z 338.0 [M+H]⁺.

Step D: 5-bromo-3,3-dimethyl-2,3-dihydro-1λ\hat{ }6, 2-benzothiazole-1,1-dione A solution of 4-bromo-2-(propan-2-yl)benzenesulfonamido acetate (500 mg, 1.48 mmol) in DMSO (5 mL) was stirred at 130° C. under N₂ for 3 hours. The cooled reaction mixture was diluted with water (50 mL), extracted with EA (50 mL×3). The organic phase was washed with brine, dried over Na₂SO₄ and concentrated. The residue was purified by silica gel chromatography (5 g column) using 0-50% EtOAc/hexane to afford 5-bromo-3,3-dimethyl-2,3-dihydro-1λ\hat{ }6, 2-benzothiazole-1,1-dione (270 mg, 0.978 mmol, 65.7%) as a clear oil. LCMS: ESI m/z 277.9 [M+H]⁺.

Step E: ({1-tert-butyl-3-[(1S,3R)-3-[(tert-butyldimethylsilyl)oxy]cyclopentyl]-1H-pyrazol-5-yl}amino)-3,3-dimethyl-2,3-dihydro-1λ\hat{ }6,2-benzothiazole-1,1-dione To a solution of 5-bromo-3,3-dimethyl-2,3-dihydro-1λ\hat{ }6, 2-benzothiazole-1,1-dione (270 mg, 0.978 mmol) and 1-tert-butyl-3-[(1S,3R)-3-[(tert-butyldimethylsilyl)oxy]cyclopentyl]-1H-pyrazol-5-amine (330 mg, 0.978 mmol) in dioxane (5 mL) was added Cs₂CO₃ (637 mg, 1.95 mmol), Xant-PHOS (56.5 mg, 0.098 mmol) and Pd₂(dba)₃ (89.5 mg, 0.098 mmol). The reaction mixture was stirred at 100° C. under N₂ for 5 hours. The reaction mixture was concentrated and the residue was purified by silica gel chromatography (5 g column) using 0-50% EtOAc/hexane to afford 5-({1-tert-butyl-3-[(1S,3R)-3-[(tert-butyldimethylsilyl)oxy]cyclopentyl]-1H-pyrazol-5-yl}amino)-3,3-dimethyl-2,3-dihydro-1λ\hat{ }6, 2-benzothiazole-1,1-dione (500 mg, 0.938 mmol, 95.9%) as a brown oil. LCMS: ESI m/z 533.4 [M+H]⁺.

Step F: 5-({1-tert-butyl-3-[(1S,3R)-3-hydroxycyclopentyl]-1H-pyrazol-5-yl}amino)-3,3-dimethyl-2,3-dihydro-1λ\hat{ }6, 2-benzothiazole-1,1-dione A solution of 5-({1-tert-butyl-3-[(1S,3R)-3-[(tert-butyldimethylsilyl)oxy]cyclopentyl]-1H-pyrazol-5-yl}amino)-3,3-dimethyl-2,3-dihydro-1λ\hat{ }6, 2-benzothiazole-1,1-dione (500 mg, 0.938 mmol) in formic acid (7 mL) was stirred at 20° C. for 30 min. The mixture was concentrated under vacuum to give 5-({1-tert-butyl-3-[(1S,3R)-3-hydroxycyclopentyl]-1H-pyrazol-5-yl}amino)-3,3-dimethyl-2,3-dihydro-1λ\hat{ }6, 2-benzothiazole-1,1-dione (370 mg, 0.884 mmol, 94.2%) as a brown oil, which was used for next step without purification. LCMS: ESI m/z 419.2 [M+H]⁺.

Step G: (1R,3S)-3-{1-tert-butyl-5-[(3,3-dimethyl-1,1-dioxo-2,3-dihydro-1λ\hat{ }6, 2-benzothiazol-5-yl)amino]-1H-pyrazol-3-yl}cyclopentyl 4-nitrophenyl carbonate To a solution of 5-({1-tert-butyl-3-[(1S,3R)-3-hydroxycyclopentyl]-1H-pyrazol-5-yl}amino)-3,3-dimethyl-2,3-dihydro-1λ\hat{ }6, 2-benzothiazole-1,1-dione (370 mg, 0.884 mmol) and 4-nitrophenyl chloroformate (267 mg, 1.32 mmol) in THF (5 mL) and DCM (5 mL) was added pyridine (0.143 mL, 1.76 mmol) and DMAP (21.6 mg, 0.177 mmol). The reaction mixture was stirred at rt for 1 hour. The mixture was concentrated and the residue was purified by silica gel chromatography (2 g column) using 0-50% EtOAc/hexane to afford (1R,3S)-3-{1-tert-butyl-5-[(3,3-dimethyl-1,1-dioxo-2,3-dihydro-1λ\hat{ }6,2-benzothiazol-5-yl)amino]-1H-pyrazol-3-yl}cyclopentyl 4-nitrophenyl carbonate (380 mg, 0.651 mmol, 73.6%) as a brown solid. LCMS: ESI m/z 584.2 [M+H]⁺.

Step H: (1R,3S)-3-{5-[(3,3-dimethyl-1,1-dioxo-2,3-dihydro-1λ\hat{ }6, 2-benzothiazol-5-yl)amino]-1H-pyrazol-3-yl}cyclopentyl 4-nitrophenyl carbonate A solution of (1R,3S)-3-{1-tert-butyl-5-[(3,3-dimethyl-1,1-dioxo-2,3-dihydro-1λ\hat{ }6, 2-benzothiazol-5-yl)amino]-1H-pyrazol-3-yl}cyclopentyl 4-nitrophenyl carbonate (380 mg, 0.651 mmol) in formic acid (10 mL) was stirred at 100° C. for 10 hours. The cooled mixture was concentrated under vacuum to give (1R,3S)-3-{5-[(3,3-dimethyl-1,1-dioxo-2,3-dihydro-1λ\hat{ }6, 2-benzothiazol-5-yl)amino]-1H-pyrazol-3-yl}cyclopentyl 4-nitrophenyl carbonate (340 mg, 0.644 mmol, 98.9%) as a brown solid, which was used for next step without purification. LCMS: ESI m/z 528.2 [M+H]⁺.

Step I: (1R,3S)-3-(3-((3,3-dimethyl-1,1-dioxido-2,3-dihydrobenzo[d]isothiazol-5-yl)amino)-1H-pyrazol-5-yl)cyclopentyl isopropylcarbamate To a solution of (1R,3S)-3-{5-[(3,3-dimethyl-1,1-dioxo-2,3-dihydro-1λ\hat{ }6,2-benzothiazol-5-yl)amino]-1H-pyrazol-3-yl}cyclopentyl 4-nitrophenyl carbonate (150 mg, 0.284 mmol) in THF (10 mL) was added DIEA (0.141 mL, 0.853 mmol) and propan-2-amine (25.2 mg, 0.037 mL, 0.426 mmol). The mixture was stirred at 20° C. for 30 min and then concentrated. The residue was purified by prep-TLC (EA) to give (1R,3S)-3-(3-((3,3-dimethyl-1,1-dioxido-2,3-dihydrobenzo[d]isothiazol-5-yl)amino)-1H-pyrazol-5-yl)cyclopentyl isopropylcarbamate (46.1 mg, 0.103 mmol, 36.2%) as a white solid. LCMS: ESI m/z 448.2 [M+H]⁺. ¹H NMR (400 MHz, DMSO) δ 11.98 (s, 1H), 9.03 (s, 1H), 7.53 (d, J=1.6 Hz, 2H), 7.48 (d, J=8.8 Hz, 1H), 7.31 (dd, J=8.8, 1.6 Hz, 1H), 6.94 (d, J=7.6 Hz, 1H), 5.69 (s, 1H), 5.00 (s, 1H), 3.58 (dd, J=13.2, 6.4 Hz, 1H), 3.13-2.99 (m, 1H), 2.49-2.44 (m, 1H), 2.02 (dd, J=15.6, 7.6 Hz, 1H), 1.96-1.83 (m, 1H), 1.78-1.70 (m, 2H), 1.68-1.59 (m, 1H), 1.47 (s, 6H), 1.03 (d, J=6.4 Hz, 6H).

Step J: amino acetate To a solution of [(tert-butoxy)carbonyl]amino acetate (1.1 g, 6.27 mmol) in DCM (4 mL) was added trifluoromethanesulfonic acid (0.558 mL, 6.28 mmol) and the reaction mixture was stirred at 20° C. for 1 hour. The reaction mixture was concentrated under vacuum to give amino acetate (500 mg, 5.99 mmol, 95.4%) as a brown solid, which was used for the next step without purification.

### Example 14. (1R,3S)-3-(3-((1,1-dioxido-2,3-dihydrobenzo[d]isothiazol-5-yl)amino)-4-fluoro-1H-pyrazol-5-yl)cyclopentyl isopropylcarbamate

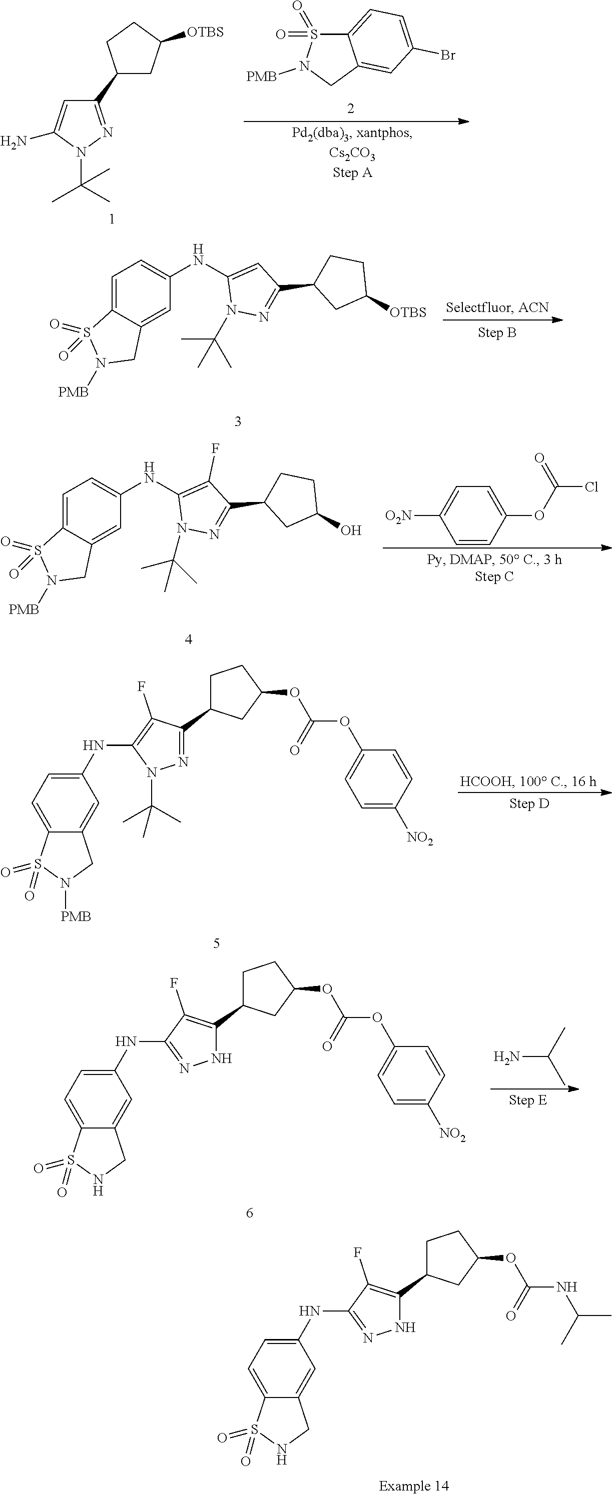

Step A: 5-((1-(tert-butyl)-3-((1S,3R)-3-((tert-butyldimethylsilyl)oxy)cyclopentyl)-1H-pyrazol-5-yl)amino)-2-(4-methoxybenzyl)-2,3-dihydrobenzo[d]isothiazole 1,1-dioxide To a solution of 1-tert-butyl-3-[(1S,3R)-3-[(tert-butyldimethylsilyl)oxy]cyclopentyl]-1H-pyrazol-5-amine (300 mg, 0.889 mmol) in dioxane (2 mL) were added 5-bromo-2-[(4-methoxyphenyl)methyl]-2,3-dihydro-λ^{A}6,2-benzothiazole-1,1-dione (392 mg, 1.06 mmol), [5-(diphenylphosphanyl)-9,9-dimethyl-9H-xanthen-4-yl]diphenylphosphane (103 mg, 0.178 mmol), Cs₂CO₃ (290 mg, 0.889 mmol) and Pd₂(dba)₃ (81.3 mg, 0.089 mmol), and the reaction was stirred at 90° C. under N₂ for 18 hr. The reaction was diluted with EA and water. The organic layer was separated, washed with further brine, and concentrated in vacuo. The residue was purified using silica gel column chromatography eluting with ethyl acetate in petroleum ether (3/1) The organic layer was collected, concentrated in vacuo, and dried to afford the title compound 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-1λ\hat{ }6,2 -benzothiazole-1,1-dione (500 mg, 0.8 mmol, 90%) as a yellow oil. LC-MS (ESI): m/z 625.3 [M+H]⁺.

Step B: 5-((1-(tert-butyl)-4-fluoro-3-((1S,3R)-3-hydroxycyclopentyl)-1H-pyrazol-5-yl)amino)-2-(4-methoxybenzyl)-2,3-dihydrobenzo[d]isothiazole 1,1-dioxide To a solution of 5-({1-tert-butyl-3-[(1S,3R)-3-[(tert-butyldimethylsilyl)oxy]cyclopentyl]-1H-pyrazol-5-yl}amino)-2-[(4-methoxyphenyl)methyl]-2,3-dihydro-1λ\hat{ }6,2-benzothiazole-1,1-dione (300 mg, 0.48 mmol) in acetonitrile (10 mL) was added selectfluor (170 mg, 0.48 mmol). The reaction mixture was stirred at 60° C. for 1 hour. After completion, the solvent was removed on vacuum to give a residue, which was purified by flash column (PE/EA=5:1) to afford title 5-({1-tert-butyl-4-fluoro-3-[(1S,3R)-3-hydroxycyclopentyl]-1H-pyrazol-5-yl}amino)-2-[(4-methoxyphenyl)methyl]-2,3-dihydro-1λ\hat{ }6,2-benzothiazole-1,1-dione (40 mg, 0.076 mmol, 15.8%) as a yellow solid. LC-MS (ESI): m/z 529.2 [M+H]⁺.

Step C: (1R,3S)-3-(1-(tert-butyl)-4-fluoro-5-((2-(4-methoxybenzyl)-1,1-dioxido-2,3-dihydrobenzo[d]isothiazol-5-yl)amino)-1H-pyrazol-3-yl)cyclopentyl (4-nitrophenyl) carbonate To a solution of 5-({1-tert-butyl-4-fluoro-3-[(1S,3R)-3-hydroxycyclopentyl]-1H-pyrazol-5-yl}amino)-2-[(4-methoxyphenyl)methyl]-2,3-dihydro-1λ\hat{ }6,2-benzothiazole-1,1-dione (40 mg, 0.076 mmol) in pyridine (5 mL) were added 4-nitrophenyl chloroformate (23 mg, 0.113 mmol), the reaction was stirred at 50° C. for 3 hours. The reaction was concentrated in vacuo. The residue was purified using silica gel column chromatography eluting with EA in PE [gradient: 0-100%] to afford the title compound (1R,3S)-3-[1-tert-butyl-4-fluoro-5-({2-[(4-methoxyphenyl)methyl]-1,1-dioxo-2,3-dihydro-1λ\hat{ }6,2-benzothiazol-5-yl}amino)-1H-pyrazol-3-yl]cyclopentyl 4-nitrophenyl carbonate (40 mg, 0.06 mmol, 80.0%) as a white solid. LC-MS (ESI): m/z 694.2 [M+H]⁺.

Step D: (1R,3S)-3-(5-((1,1-dioxido-2,3-dihydrobenzo[d]isothiazol-5-yl)amino)-4-fluoro-1H-pyrazol-3-yl)cyclopentyl (4-nitrophenyl) carbonate A solution of (1R,3S)-3-[1-tert-butyl-4-fluoro-5-({2-[(4-methoxyphenyl)methyl]-1,1-dioxo-2,3-dihydro-1λ\hat{ }6,2-benzothiazol-5-yl}amino)-1H-pyrazol-3-yl]cyclopentyl 4-nitrophenyl carbonate (40 mg, 0.06 mmol) in HCOOH (10 mL) was stirred at 100° C. overnight. The reaction was concentrated in vacuo to afford the title compound (1R,3S)-3-{5-[(1,1-dioxo-2,3-dihydro-1λ\hat{ }6,2-benzothiazol-5-yl)amino]-4-fluoro-1H-pyrazol-3-yl}cyclopentyl 4-nitrophenyl carbonate (30 mg, 0.06 mmol, crude) as a white solid. LC-MS (ESI): m/z 518 [M+H]⁺.

Step E: (1R,3S)-3-(3-((1,1-dioxido-2,3-dihydrobenzo[d]isothiazol-5-yl)amino)-4-fluoro-1H-pyrazol-5-yl)cyclopentyl isopropylcarbamate To a solution of (1R,3S)-3-{5-[(1,1-dioxo-2,3-dihydro-1λ\hat{ }6,2-benzothiazol-5-yl)amino]-4-fluoro-1H-pyrazol-3-yl}cyclopentyl 4-nitrophenyl carbonate (30 mg, 0.06 mmol) in THF (2 mL) was added propan-2-amine (300 mg, 5.08 mmol) and the reaction was stirred at 25° C. for 2 hr. The reaction concentrated in vacuo. The residue was purified by prep-HPLC (FA condition) to afford the title compound (1R,3S)-3-{5-[(1,1-dioxo-2,3-dihydro-1λ\hat{ }6,2-benzothiazol-5-yl)amino]-4-fluoro-1H-pyrazol-3-yl}cyclopentyl N-(propan-2-yl)carbamate (1.2 mg, 4.0%) as a white solid. LC-MS (ESI): m/z 438.2 [M+H]⁺. ¹H NMR (400 MHz, Methanol-d₄) δ 7.51 (d, J=8.4 Hz, 1H), 7.17 (s, 2H), 5.10 (s, 1H), 4.34 (s, 2H), 3.79-3.62 (m, 1H), 3.25-3.15 (m, 1H), 2.61-2.52 (m, 1H), 2.23-1.80 (m, 5H), 1.31 (d, J=6.4 Hz, 1H), 1.11 (d, J=6.4 Hz, 6H).

### Example 15: (1R,3S)-3-{5-[(3-methoxy-1,1-dioxo-2,3-dihydro-1λ\hat{ }6-benzothiophen-5-yl)amino]-2H-pyrazol-3-yl}cyclopentyl (prop-2-ylamino)methanoate

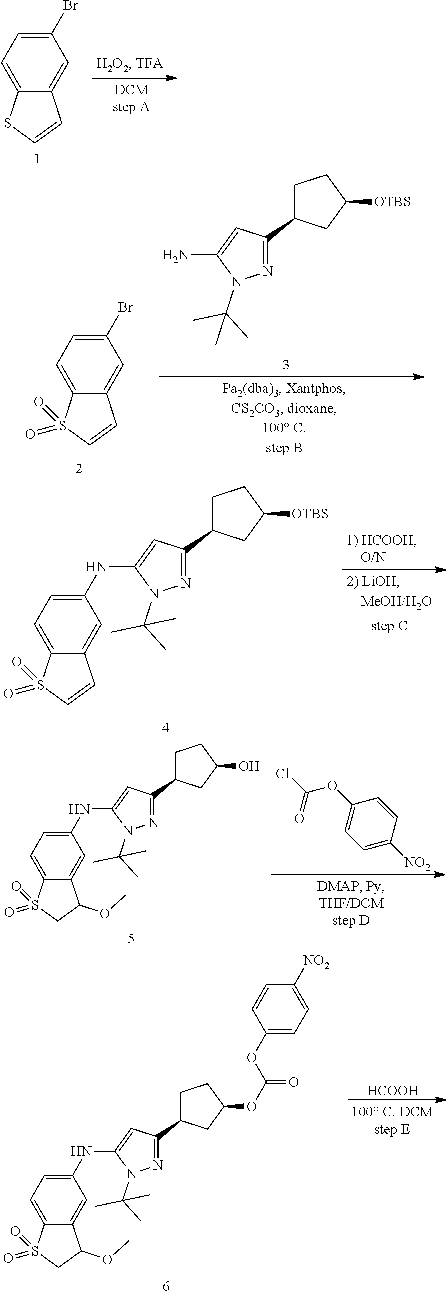

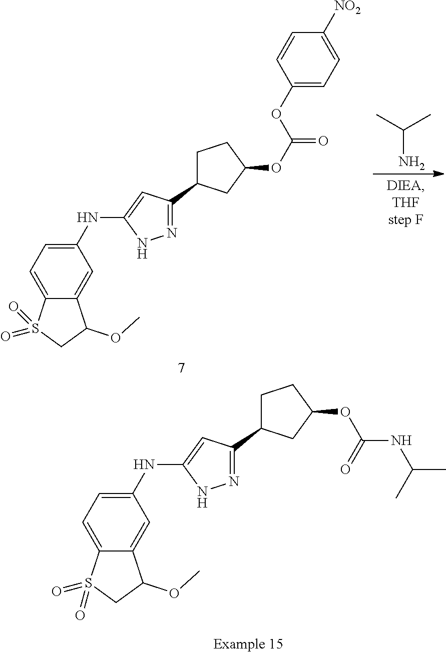

Step A: 5-bromo-1λ6-benzothiophene-1,1-dione To a stirred solution of 5-bromobenzothiophene (2 g, 9.39 mmol) in DCM (16 mL) was added H₂02(4 mL, 39.2 mmol, 30% in H₂O) and TFA (4 mL, 53.8 mmol) at rt. After stirred at rt for 5 h, the mixture was poured into aqueous Na₂S₂O₃ and extracted with DCM (30 mL×2). The combined organic phase was washed with brine, dried over Na₂SO₄, filtered and concentrated. The residue was purified by chromatography (silica gel, 0-40%, EA in PE) to give 5-bromo-1λ6-benzothiophene-1,1-dione (1.7 g, 6.94 mmol, 73.9%) as a white solid.

Step B: 5-{[2-(2-methylprop-2-yl)-5-[(1S,3R)-3-{[(2-methylprop-2-yl)diphenylsilyl]oxy}cyclopentyl]pyrazol-3-yl]amino}-1λ6-benzothiophene-1,1-dione To a solution of 5-[(1S,3R)-3-{[dimethyl(2-methylprop-2-yl)silyl]oxy}cyclopentyl]-2-(2-methylprop-2-yl)pyrazol-3-amine (300 mg, 0.889 mmol) was added 5-bromo-16-benzothiophene-1,1-dione (191 mg, 0.779 mmol), Pd₂(dba)₃ (59.4 mg, 0.065 mmol), Xantphos (75.1 mg, 0.130 mmol) and Cs₂CO₃ (529 mg, 1.62 mmol). The reaction was purged with N₂ and stirred at 100° C. under N₂ for 1.5 h. The reaction was diluted with EA and water. The organic layer was separated, washed with further brine, and concentrated in vacuo. The residue was purified using silica gel column chromatography eluting with ethyl acetate in petroleum ether (2/1) The organic layer was collected, concentrated in vacuo, and dried to afford the title compound 5-{[2-(2-methylprop-2-yl)-5-[(1S,3R)-3-{[(2-methylprop-2-yl)diphenylsilyl]oxy}cyclopentyl]pyrazol-3-yl]amino}-1λ6-benzothiophene-1,1-dione (350 mg, 0.559 mmol, 86.2%) as a yellow oil. LCMS: ESI m/z 626 [M+H]⁺.

Step C: 5-({5-[(1S,3R)-3-hydroxycyclopentyl]-2-(2-methylprop-2-yl)pyrazol-3-yl}amino)-3-methoxy-2,3-dihydro-1λ⁶-benzothiophene-1,1-dione A mixture of 5-{[2-(2-methylprop-2-yl)-5-[(1S,3R)-3-{[(2-methylprop-2-yl)diphenylsilyl]oxy}cyclopentyl]pyrazol-3-yl]amino}-1λ⁶-benzothiophene-1,1-dione (350 mg, 0.559 mmol) in HCOOH (4 mL) was stirred at RT overnight and then concentrated under reduced pressure. The residue was treated with a mixture of LiOH—H₂O (0.062 mL, 2.24 mmol) in MeOH (2 mL)/H₂O (2 mL). The resulting mixture was stirred at rt for 1 h and then concentrated under reduced pressure. The following mixture was poured into water and extracted with EtOAc. The combined organic phase was washed with brine, dried with Na₂SO₄, filtered and concentrated. The residue was purified by chromatography (silica gel, 0-80%, EA in PE) to give 5-({5-[(1S,3R)-3-hydroxycyclopentyl]-2-(2-methylprop-2-yl)pyrazol-3-yl}amino)-3-methoxy-2,3-dihydro-1λ⁶-benzothiophene-1,1-dione (220 mg, 0.524 mmol, 93.8%) as a white solid. LCMS: m/z 420 [M+H]⁺. ¹H NMR (400 MHz, DMSO) δ 8.24 (s, 1H), 7.53 (d, J=8.8 Hz, 1H), 6.80 (dd, J=8.8, 2.0 Hz, 1H), 6.71 (d, J=2.0 Hz, 1H), 5.99 (s, 1H), 5.07 (dd, J=6.8, 3.6 Hz, 1H), 4.56 (d, J=4.4 Hz, 1H), 4.19-4.13 (m, 1H), 3.87 (dd, J=13.6, 6.8 Hz, 1H), 3.47 (dd, J=13.6, 3.6 Hz, 1H), 3.35 (s, 3H), 2.99-2.89 (m, 1H), 2.25-2.18 (m, 1H), 1.90-1.83 (m, 1H), 1.77-1.68 (m, 2H), 1.65-1.50 (m, 1H), 1.50 (s, 9H).

Step D: (1R,3S)-3-{5-[(3-methoxy-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate To a solution of 5-({5-[(1S,3R)-3-hydroxycyclopentyl]-2-(2-methylprop-2-yl)pyrazol-3-yl}amino)-3-methoxy-2,3-dihydro-X⁶-benzothiophene-1,1-dione (220 mg, 0.524 mmol) in THF (3 mL) and DCM (3 mL) were added 4-nitrophenyl chloromethanoate (317 mg, 1.58 mmol), DMAP (6.41 mg, 0.052 mmol) and Py (0.127 mL, 1.57 mmol), the reaction was stirred at rt for 2 h. The reaction was concentrated in vacuo. The residue was purified using silica gel column chromatography eluting with EA in PE [gradient: 0-50%]. to afford the title compound (1R,3S)-3-{5-[(3-methoxy-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate (210 mg, 0.359 mmol, 68.5%) as a white solid. LCMS: ESI 585 [M+H]⁺

Step E: (1R,3S)-3-{5-[(3-methoxy-1,1-dioxo-2,3-dihydro-1λ⁶-benzothiophen-5-yl)amino]-1H-pyrazol-3-yl}cyclopentyl [(4-nitrophenyl)oxy]methanoate A solution of (1R,3S)-3-{5-[(3-methoxy-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate (100 mg, 0.171 mmol) in HCOOH (2 mL) was stirred at 100° C. overnight. The reaction was concentrated in vacuo to afford the title compound (1R,3S)-3-{5-[(3-methoxy-1,1-dioxo-2,3-dihydro-1λ⁶-benzothiophen-5-yl)amino]-1H-pyrazol-3-yl}cyclopentyl [(4-nitrophenyl)oxy]methanoate (88 mg, 0.166 mmol, 97.4%) as a yellow oil, which was used into next step without further purification. LCMS: ESI m/z 529 [M+H]⁺Step F: (1R,3S)-3-{5-[(3-methoxy-1,1-dioxo-2,3-dihydro-1λ⁶-benzothiophen-5-yl)amino]-2H-pyrazol-3-yl}cyclopentyl (prop-2-ylamino)methanoate

To a solution of (1R,3S)-3-{5-[(3-methoxy-1,1-dioxo-2,3-dihydro-1λ⁶-benzothiophen-5-yl)amino]-1H-pyrazol-3-yl}cyclopentyl [(4-nitrophenyl)oxy]methanoate (88 mg, 0.166 mmol) in THF (2 mL) was added isopropylamine (0.043 mL, 0.499 mmol) and DIPEA (64.6 mg, 0.499 mmol) at rt. After stirred at rt for 30 min, the mixture was concentrated. The residue was purified by prep-HPLC (C18, 20˜ 95% MeCN in H₂O with 0.1% HCOOH) to (1R,3S)-3-{5-[(3-methoxy-1,1-dioxo-2,3-dihydro-1λ⁶-benzothiophen-5-yl)amino]-2H-pyrazol-3-yl}cyclopentyl (prop-2-ylamino)methanoate (37.6 mg, 0.084 mmol, 50.4%) as a white solid. LCMS: ESI m/z 449.2 [M+H]⁺. 1H NMR (400 MHz, DMSO) δ 11.96 (s, 1H), 9.10 (s, 1H), 7.70 (s, 1H), 7.53 (d, J=8.4 Hz, 1H), 7.39 (d, J=8.4 Hz, 1H), 6.94 (d, J=7.6 Hz, 1H), 5.68 (s, 1H), 5.10 (dd, J=6.8, 3.6 Hz, 1H), 5.00 (s, 1H), 3.88 (dd, J=13.6, 6.8 Hz, 1H), 3.63-3.55 (m, 1H), 3.48 (dd, J=13.6, 3.6 Hz, 1H), 3.39 (s, 3H), 3.10-3.00 (m, 1H), 2.48-2.42 (m, 1H), 2.02 (dd, J=15.2, 7.6 Hz, 1H), 1.96-1.85 (m, 1H), 1.76-1.53 (m, 3H), 1.03 (d, J=6.4 Hz, 6H).

### Example 16: (1s,4s)-4-{5-[(1,1-dioxo-2,3-dihydro-1λ\hat{ }6-benzothiophen-5-yl)amino]-2H-pyrazol-3-yl}cyclohexyl (prop-2-ylamino)methanoate

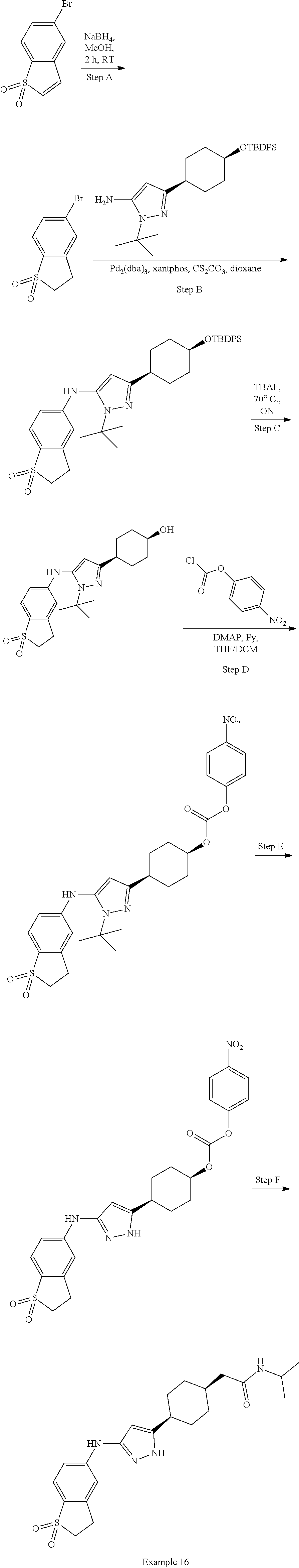

Step A: 5-bromo-2,3-dihydro-16-benzothiophene-1,1-dione To a solution of 5-bromo-1λ6-benzothiophene-1,1-dione (1.1 g, 4.49 mmol) in MeOH (50 mL) was added NaBH₄ (0.30 g, 8.98 mmol). The reaction mixture was stirred at room temperature for 3 h. The reaction was diluted with EA and water. The organic layer was separated, washed with brine and concentrated in vacuo. The residue was purified using silica gel column chromatography eluted with ethyl acetate in petroleum ether (gradient: 0-30%) to afford the title compound 5-bromo-2,3-dihydro-1λ6-benzothiophene-1,1-dione (1.1 g, 4.45 mmol, 99.2%) as a white solid. LCMS: m/z 247.11 [M+H]⁺.

Step B: 5-({5-[(1s,4s)-4-{[(2-methylprop-2-yl)diphenylsilyl]oxy}cyclohexyl]-2-(2-methylprop-2-yl)pyrazol-3-yl}amino)-2,3-dihydro-1λ\hat{ }6-benzothiophene-1,1-dione To a solution of 5-bromo-2,3-dihydro-16-benzothiophene-1,1-dione (200 mg, 0.809 mmol) in dioxane (10 mL) were added 5-[(1s,4s)-4-{[(2-methylprop-2-yl)diphenylsilyl]oxy}cyclohexyl]-2-(2-methylprop-2-yl)pyrazol-3-amine (385 mg, 0.809 mmol), Cs₂CO₃ (791 mg, 2.43 mmol), Pd₂(dba)₃ (74.1 mg, 0.081 mmol) and Xant-PHOS (93.7 mg, 0.162 mmol). The reaction was stirred at 100° C. under N₂ for 3 hr. The cooled reaction was diluted with EA and water. The organic layer was separated, washed with brine and concentrated in vacuo. The residue was purified using silica gel column chromatography eluted with ethyl acetate in petroleum ether (gradient:0-30%) to afford the title compound 5-({5-[(1s,4s)-4-{[(2-methylprop-2-yl)diphenylsilyl]oxy}cyclohexyl]-2-(2-methylprop-2-yl)pyrazol-3-yl}amino)-2,3-dihydro-1λ6-benzothiophene-1,1-dione (240 mg, 0.374 mmol, 46.2%) as a yellow solid. LCMS: m/z 641.95 [M+H]⁺.

Step C: 5-({5-[(1s,4s)-4-hydroxycyclohexyl]-2-(2-methylprop-2-yl)pyrazol-3-yl}amino)-2,3-dihydro-1λ\hat{ }6-benzothiophene-1,1-dione A solution of 5-({5-[(1s,4s)-4-{[(2-methylprop-2-yl)diphenylsilyl]oxy}cyclohexyl]-2-(2-methylprop-2-yl)pyrazol-3-yl}amino)-2,3-dihydro-1λ6-benzothiophene-1,1-dione (240 mg, 0.374 mmol) in TBAF (5 mL, 1M in THF) was stirred 70° C. overnight. The cooled reaction was diluted with EA and water. The organic layer was separated, washed with brine and concentrated in vacuo. The residue was purified using silica gel column chromatography eluted with methanol in dichloroform (gradient:0-10%) to afford the title compound 5-({5-[(1s,4s)-4-hydroxycyclohexyl]-2-(2-methylprop-2-yl)pyrazol-3-yl}amino)-2,3-dihydro-1λ6-benzothiophene-1,1-dione (150 mg, 0.372 mmol, 99.4%) as a yellow oil. LCMS: m/z 403.0 [M+H]⁺.

Step D: (1s,4s)-4-{5-[(1,1-dioxo-2,3-dihydro-1λ\hat{ }6-benzothiophen-5-yl)amino]-1-(2-methylprop-2-yl)pyrazol-3-yl}cyclohexyl [(4-nitrophenyl)oxy]methanoate To a solution of 5-({5-[(1s,4s)-4-hydroxycyclohexyl]-2-(2-methylprop-2-yl)pyrazol-3-yl}amino)-2,3-dihydro-1λ6-benzothiophene-1,1-dione (180 mg, 0.446 mmol) in THF (5 mL) and DCM (5 mL) were added 4-nitrophenyl chloromethanoate (135 mg, 0.669 mmol), DMAP (5.45 mg, 0.045 mmol) and Py (70.6 mg, 0.892 mmol). The reaction was stirred at room temperature overnight. The reaction was diluted with EA and water. The organic layer was separated, washed with saturated NaCl solution and concentrated in vacuo. The residue was purified using silica gel column chromatography eluted with ethyl acetate in petroleum ether (gradient: 0-30%) to afford the title compound (1s,4s)-4-{5-[(1,1-dioxo-2,3-dihydro-1λ6-benzothiophen-5-yl)amino]-1-(2-methylprop-2-yl)pyrazol-3-yl}cyclohexyl [(4-nitrophenyl)oxy]methanoate (120 mg, 0.211 mmol, 47.3%) as a yellow solid. LCMS: m/z 568.64 [M+H]⁺.

Step E: (1R,3S)-3-{5-[(4-methyl-1,1-dioxo-4H-16-benzo[2,1-e][1,2,4]thiadiazin-6-yl)amino]-2H-pyrazol-3-yl}cyclopentyl (prop-2-ylamino)methanoate A solution of (1s,4s)-4-{5-[(1,1-dioxo-2,3-dihydro-1λ6-benzothiophen-5-yl)amino]-1-(2-methylprop-2-yl)pyrazol-3-yl}cyclohexyl [(4-nitrophenyl)oxy]methanoate (60 mg, 0.106 mmol) in formic acid (5 mL) was stirred at 100° C. overnight. The reaction was concentrated in vacuo to afford the title compound (1s,4s)-4-{5-[(1,1-dioxo-2,3-dihydro-1λ6-benzothiophen-5-yl)amino]-2H-pyrazol-3-yl}cyclohexyl [(4-nitrophenyl)oxy]methanoate (50 mg, 0.098 mmol, 92.5%) as a yellow oil. LCMS: m/z 512.54 [M+H]⁺.

Step F: (1s,4s)-4-{5-[(1,1-dioxo-2,3-dihydro-1λ\hat{ }6-benzothiophen-5-yl)amino]-2H-pyrazol-3-yl}cyclohexyl (prop-2-ylamino)methanoate A solution of (1s,4s)-4-{5-[(1,1-dioxo-2,3-dihydro-1λ\hat{ }6-benzothiophen-5-yl)amino]-2H-pyrazol-3-yl}cyclohexyl [(4-nitrophenyl)oxy]methanoate (50 mg, 0.098 mmol) in propan-2-amine (3 mL, 35.0 mmol) was stirred at room temperature for 1 hr. The reaction was concentrated in vacuo. The residue was purified by prep-HPLC (C18, 0-70% acetonitrile in H₂O) to afford the title compound (1s,4s)-4-{5-[(1,1-dioxo-2,3-dihydro-1λ6-benzothiophen-5-yl)amino]-2H-pyrazol-3-yl}cyclohexyl (prop-2-ylamino)methanoate (20.9 mg, 0.048 mmol, 49.5%) as a white solid. LCMS: m/z 432.54 [M+H]⁺. ¹H NMR (400 MHz, DMSO) δ 11.90 (s, 1H), 9.00 (s, 1H), 7.47 (d, J=8.8 Hz, 2H), 7.29 (d, J=9.2 Hz, 1H), 6.88 (s, 1H), 5.67 (s, 1H), 4.75 (s, 1H), 3.63-3.50 (m, 1H), 3.47 (t, J=6.8 Hz, 2H), 3.24 (t, J=6.8 Hz, 2H), 2.68 (s, 1H), 1.83-1.75 (m, 4H), 1.75-1.58 (m, 4H), 1.05 (d, J=6.4 Hz, 6H).

### Example 17: (1R,3S)-3-(3-((1,1-dioxido-2,3-dihydrobenzo[d]isothiazol-6-yl)amino)-1H-pyrazol-5-yl)cyclopentyl isopropylcarbamate

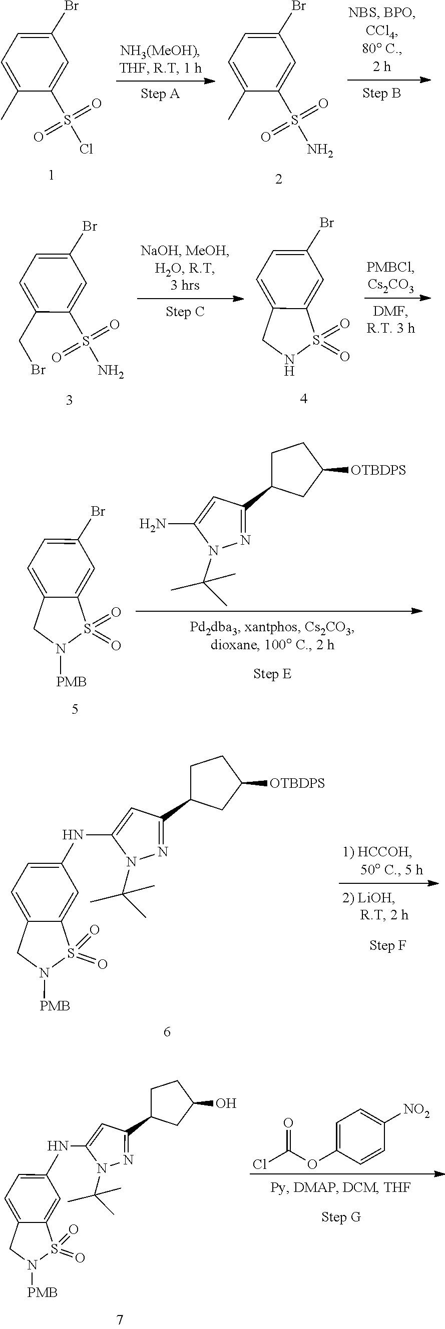

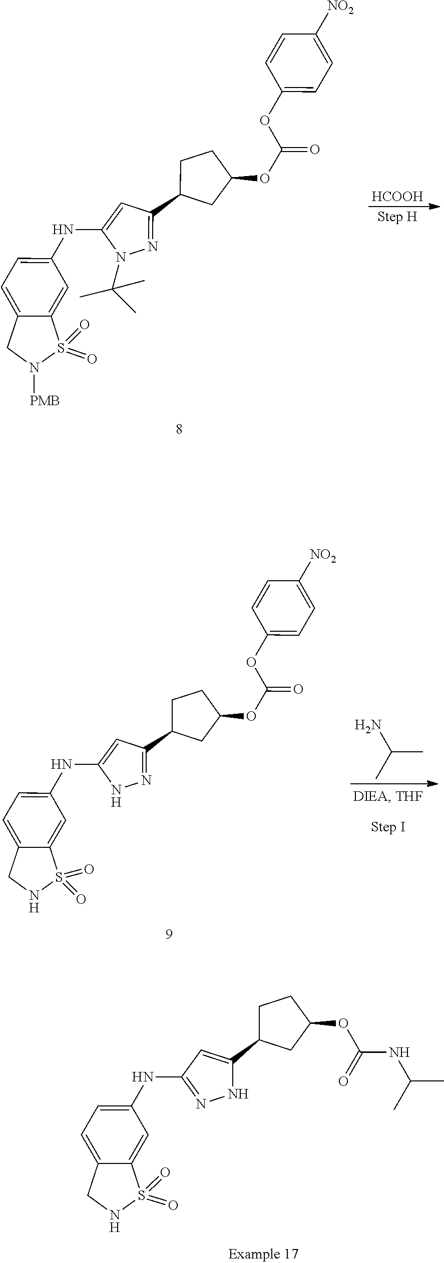

Step A: 5-bromo-2-methylbenzenesulfonamide To a solution of 5-bromo-2-methylbenzenesulfonyl chloride (3.70 g, 13.7 mmol) in THF (50 mL) was added NH3-MeOH (2 mol/L, 108 mL, 216 mmol) and the mixture was stirred at room temperature for 1 hr. The reaction was complete detected by LCMS. The reaction mixture was concentrated to afford 5-bromo-2-methylbenzenesulfonamide (3.30 g, 13.2 mmol, 96.0%) as a white solid. LCMS: ESI m/z 251.12 [M+H]⁺.

Step B: 5-bromo-2-(bromomethyl)benzenesulfonamide To a solution of 5-bromo-2-methylbenzenesulfonamide (3.30 g, 13.2 mmol) in CCl₄ (40 mL) was added NBS (3.52 g, 19.7 mmol) and BPO (1.60 g, 6.60 mmol) and the mixture was stirred at 80° C. for 2 hrs. The reaction was complete detected by TLC. The reaction mixture was dissolved in EA (50 mL), washed with H₂O (20 mL) and brine, dried over sodium sulfate, filtered, concentrated. The residue was purified by silica gel chromatography (eluting with 0-30% EA in PE) to afford 5-bromo-2-(bromomethyl)benzenesulfonamide (2.90 g, 8.81 mmol, 67.6%) as a white solid.

Step C: 6-bromo-2,3-dihydrobenzo[d]isothiazole 1,1-dioxide To a solution of 5-bromo-2-(bromomethyl)benzenesulfonamide (2.90 g, 8.81 mmol) in MeOH (45 mL) and H₂O (11.3 mL) was added NaOH (0.710 g, 17.6 mmol) and the mixture was stirred at room temperature for 3 hrs. The reaction was complete detected by LCMS. The reaction mixture was concentrated in vacuo and purified by silica gel chromatography (eluting with 0-50% EA in PE) to afford 6-bromo-2,3-dihydrobenzo[d]isothiazole 1,1-dioxide (880 mg, 3.55 mmol, 40.2%) as a white solid. LCMS: ESI m/z 248.10 [M+H]⁺.

Step D: 6-bromo-2-(4-methoxybenzyl)-2,3-dihydrobenzo[d]isothiazole 1,1-dioxide To a solution of 6-bromo-2,3-dihydrobenzo[d]isothiazole 1,1-dioxide (450 mg, 1.81 mmol) in DMF (10 mL) was added PMBC1 (426 mg, 2.72 mmol) and Cs₂CO₃ (1.18 mg, 3.63 mmol) and the mixture was stirred at room temperature for 3 hrs. The reaction was complete detected by LCMS. The reaction mixture was dissolved in EA (50 mL), washed with H₂O (10 mL) and brine, dried over sodium sulfate, filtered, concentrated, and purified by silica gel chromatography (eluting with 0-80% EA in PE) to afford 6-bromo-2-(4-methoxybenzyl)-2,3-dihydrobenzo[d]isothiazole 1,1-dioxide (250 mg, 0.679 mmol, 37.4%) as a faint yellow solid. LCMS: ESI m/z 368.25 [M+H]⁺.

Step E: 6-((1-(tert-butyl)-3-((1S,3R)-3-((tert-butyldiphenylsilyl)oxy)cyclopentyl)-1H-pyrazol-5-yl)amino)-2-(4-methoxybenzyl)-2,3-dihydrobenzo[d]isothiazole 1,1-dioxide To a solution of 6-bromo-2-(4-methoxybenzyl)-2,3-dihydrobenzo[d]isothiazole 1,1-dioxide (250 mg, 0.679 mmol) in dioxane (8 mL) was added 1-(tert-butyl)-3-((1S,3R)-3-((tert-butyldiphenylsilyl)oxy)cyclopentyl)-1H-pyrazol-5-amine (150 mg, 0.325 mmol), Cs₂CO₃ (317 mg, 0.975 mmol), Pd₂(dba)₃ (29.8 mg, 0.0320 mmol) and Xantphos (37.6 mg, 0.065 mmol). The reaction mixture was stirred at 100° C. under N₂ atmosphere for 1 hr. The reaction was complete detected by LCMS. The cooled reaction mixture was dissolved in EA (50 mL), washed with H₂O (20 mL) and brine, dried over sodium sulfate, filtered, concentrated, and purified by silica gel chromatography (eluting with 0-80% EA in PE) to afford 6-((1-(tert-butyl)-3-((1S,3R)-3-((tert-butyldiphenylsilyl)oxy)cyclopentyl)-1H-pyrazol-5-yl)amino)-2-(4-methoxybenzyl)-2,3-dihydrobenzo[d]isothiazole 1,1-dioxide (150 mg, 0.200 mmol, 61.6%) as a white solid. LCMS: ESI m/z 749.06 [M+H]⁺.

Step F: 6-((1-(tert-butyl)-3-((1S,3R)-3-hydroxycyclopentyl)-1H-pyrazol-5-yl)amino)-2-(4-methoxybenzyl)-2,3-dihydrobenzo[d]isothiazole 1,1-dioxide A solution of 6-((1-(tert-butyl)-3-((1S,3R)-3-((tert-butyldiphenylsilyl)oxy)cyclopentyl)-1H-pyrazol-5-yl)amino)-2-(4-methoxybenzyl)-2,3-dihydrobenzo[d]isothiazole 1,1-dioxide (150 mg, 0.200 mmol) in formic acid (2 mL) was stirred at 50° C. for 5 hrs. The reaction was complete detected by TLC. The solvents were removed under vacuum. Then add EtOH (2 mL), H₂O (2 mL) and Lithium Hydroxide Monohydrate (25.2 mg, 0.601 mmol) to alkaline pH. The reaction mixture was stirred at room temperature for 1 hr. LCMS showed the reaction was completed. The reaction mixture was dissolved in EA (50 mL), washed with H₂O (10 mL) and sbrine, dried over sodium sulfate, filtered, concentrated. The residue was purified by silica gel chromatography (eluting with 0-80% EA in PE) to afford 6-((1-(tert-butyl)-3-((1S,3R)-3-hydroxycyclopentyl)-1H-pyrazol-5-yl)amino)-2-(4-methoxybenzyl)-2,3-dihydrobenzo[d]isothiazole 1,1-dioxide (100 mg, 0.196 mmol, 97.8%) as a faint yellow oil. LCMS: ESI m/z 511.65 [M+H]⁺.

Step G: (1R,3S)-3-(1-(tert-butyl)-5-((2-(4-methoxybenzyl)-1,1-dioxido-2,3-dihydrobenzo[d]isothiazol-6-yl)amino)-1H-pyrazol-3-yl)cyclopentyl (4-nitrophenyl) carbonate A suspension of 6-((1-(tert-butyl)-3-((1S,3R)-3-hydroxycyclopentyl)-1H-pyrazol-5-yl)amino)-2-(4-methoxybenzyl)-2,3-dihydrobenzo[d]isothiazole 1,1-dioxide (100 mg, 0.196 mmol), DMAP (2.39 mg, 0.0200 mmol), Py (0.0480 mL, 0.588 mmol) and 4-nitrophenyl chloromethanoate (198 mg, 0.980 mmol) in DCM (2 mL) and THF (2 mL) was stirred at room temperature for 3 hrs. The solvents were removed under vacuum. The residue was dissolved in EA (50 mL), washed with H₂O (5 mL) andbrine, dried over sodium sulfate, filtered, concentrated. The residue was purified by silica gel chromatography (eluting with 0-80% EA in PE) to afford (1R,3S)-3-(1-(tert-butyl)-5-((2-(4-methoxybenzyl)-1,1-dioxido-2,3-dihydrobenzo[d]isothiazol-6-yl)amino)-1H-pyrazol-3-yl)cyclopentyl (4-nitrophenyl) carbonate (100 mg, 0.148 mmol, 75.6%) as a faint yellow oil. LCMS: ESI m/z 676.76 [M+H]⁺.

Step H: (1R,3S)-3-(5-((1,1-dioxido-2,3-dihydrobenzo[d]isothiazol-6-yl)amino)-1H-pyrazol-3-yl)cyclopentyl (4-nitrophenyl) carbonate A solution of (1R,3S)-3-(1-(tert-butyl)-5-((2-(4-methoxybenzyl)-1,1-dioxido-2,3-dihydrobenzol[d]isothiazol-6-yl)amino)-1H-pyrazol-3-yl)cyclopentyl (4-nitrophenyl) carbonate (100 mg, 0.148 mmol) was dissolved in formic acid (2 mL) and stirred at 100° C. overnight. LCMS showed the reaction was completed. The cooled reaction mixture was concentrated to give (1R,3S)-3-(5-((1,1-dioxido-2,3-dihydrobenzo[d]isothiazol-6-yl)amino)-1H-pyrazol-3-yl)cyclopentyl (4-nitrophenyl) carbonate(70.0 mg, 0.140 mmol, 94.7%) as a yellow oil. LCMS: ESI m/z 500.50 [M+H]⁺.

Step I: (1R,3S)-3-(3-((1,1-dioxido-2,3-dihydrobenzo[d]isothiazol-6-yl)amino)-1H-pyrazol-5-yl)cyclopentyl isopropylcarbamate To a suspension of (1R,3S)-3-(5-((1,1-dioxido-2,3-dihydrobenzo[d]isothiazol-6-yl)amino)-1H-pyrazol-3-yl)cyclopentyl (4-nitrophenyl) carbonate(70.0 mg, 0.140 mmol) in THF (2 mL) was added propan-2-amine (0.0360 mL, 0.420 mmol) and DIEA (0.0690 mL, 0.420 mmol). The reaction mixture was stirred at room temperature for 2 hrs. LCMS showed the reaction had been completed. The reaction mixture was dissolved in EA (50 mL), washed with H₂O (10 mL) and brine, dried over sodium sulfate, filtered, concentrated. The residue was purified by silica gel chromatography (eluting with 12% MeOH in DCM) and pre-HPLC (C18, 15˜ 95% MeCN in H₂O with 0.1% FA) to afford (1R,3S)-3-(3-((1,1-dioxido-2,3-dihydrobenzo[d]isothiazol-6-yl)amino)-1H-pyrazol-5-yl)cyclopentyl isopropylcarbamate (2.00 mg, 0.00500 mmol, 3.40%) as a white solid. LCMS: ESI m/z 420.50 [M+H]⁺. ¹H NMR (400 MHz, DMSO) δ 8.90 (s, 1H), 7.97 (s, 1H), 7.62 (br s, 1H), 7.39 (d, J=8.4 Hz, 1H), 7.31 (d, J=8.4 Hz, 1H), 6.95 (d, J=6.8 Hz, 1H), 5.65 (s, 1H), 5.00 (s, 1H), 4.27 (s, 2H), 3.11-3.05 (m, 1H), 2.48-2.40 (m, 1H), 2.04-1.89 (m, 2H), 1.77-1.53 (m, 3H), 1.04 (d, J=6.0 Hz, 6H).

### Example 18: (1R,3S)-3-(3-((4-methyl-1,1-dioxido-3-oxo-3,4-dihydro-2H-benzo[e][1,2,4]thiadiazin-6-yl)amino)-1H-pyrazol-5-yl)cyclopentyl isopropylcarbamate

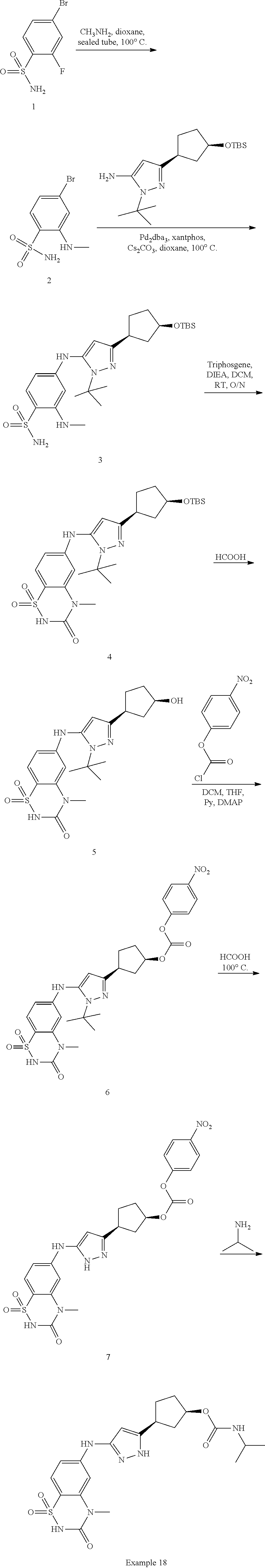

Step A: 4-bromo-2-(methylamino)benzene-1-sulfonamide To a solution of 4-bromo-2-fluorobenzene-1-sulfonamide (300 mg, 1.2 mmol) in 1,4-dioxane (4 mL) were added CH₃NH₂ (3 mL, 2M in THF). The reaction was stirred at 100° C. in a sealed tube for 18 h. TLC and LCMS indicated the reaction was over. The cooled reaction mixture was concentrated and the residue was purified by a silica gel column chromatography eluted with ethyl acetate in petroleum ether (gadient: 0-30%) to afford the title compound 4-bromo-2-(methylamino)benzene-1-sulfonamide (260 mg, 1.0 mmol, 83.0%) as a yellow solid. LCMS m/z: 266 [M+H]⁺

Step B: 4-({5-[(1S,3R)-3-{[dimethyl(2-methylprop-2-yl)silyl]oxy}cyclopentyl]-2-(2-methylprop-2-yl)pyrazol-3-yl}amino)-2-(methylamino)benzene-1-sulfonamide To a solution of 4-bromo-2-(methylamino)benzene-1-sulfonamide (210 mg, 0.792 mmol), 5-[(1S,3R)-3-{[dimethyl(2-methylprop-2-yl)silyl]oxy}cyclopentyl]-2-(2-methylprop-2-yl)pyrazol-3-amine (268 mg, 0.8 mmol), Xantphos (68 mg, 0.12 mmol) and Cs₂CO₃ (774 mg, 2.4 mmol) in 1,4-dioxane (10 mL) was added Pd₂(dba)₃ (72 mg, 0.08 mmol). The reaction was stirred at 100° C. under N₂ atmosphere for 5 h. TLC and LCMS indicted the reaction was over. The cooled reaction mixture was poured into ice-water, adjust pH to 5-6 with 1N HCl, extracted with EA (20 mL*3), the combined organic layer was washed with brine, dried over Na₂SO₄, filtered and concentrated. The residue was purified by a silica column eluted with PE: EA (gradient: 0-50%) to give the desire product 4-({5-[(1S,3R)-3-{[dimethyl(2-methylprop-2-yl)silyl]oxy}cyclopentyl]-2-(2-methylprop-2-yl)pyrazol-3-yl}amino)-2-(methylamino)benzene-1-sulfonamide (130 mg, 0.25 mmol, 31%) as a yellow solid. LCMS m/z:522 [M+H]⁺

Step C: 6-({5-[(1S,3R)-3-{[dimethyl(2-methylprop-2-yl)silyl]oxy}cyclopentyl]-2-(2-methylprop-2-yl)pyrazol-3-yl}amino)-4-methyl-3-oxo-3,4-dihydro-2H-16-benzo[2,1-e][1,2,4]thiadiazine-1,1-dione To a solution of 4-({5-[(1S,3R)-3-{[dimethyl(2-methylprop-2-yl)silyl]oxy}cyclopentyl]-2-(2-methylprop-2-yl)pyrazol-3-yl}amino)-2-(methylamino)benzene-1-sulfonamide (430 mg, 0.8 mmol) in DMF (10 mL) was added NaH (165 mg, 4.1 mmol, 60% in mineral oil) at 0° C. and the reaction was stirred for 30 min. Triphosgene (244 mg, 0.8 mmol) was added into the reaction and the reaction was stirred at room temperature overnight. The reaction was poured into ice-water and adjust pH to 5-6 with 1N HCl, extracted with EA, the organic layer was washed with brine, dried over Na₂SO₄, filtered and concentrated. The residue was purified by silica gel column chromatography eluted with methanol in dichloroform (gadient: 0-10%) to afford the title compound 6-({5-[(1S,3R)-3-{[dimethyl(2-methylprop-2-yl)silyl]oxy}cyclopentyl]-2-(2-methylprop-2-yl)pyrazol-3-yl}amino)-4-methyl-3-oxo-3,4-dihydro-2H-1λ6-benzo[2,1-e][1,2,4]thiadiazine-1,1-dione (280 mg, 0.5 mmol, 62%) as a gray solid. LCMS m/z:548 [M+H]⁺.

Step D: 6-({5-[(1S,3R)-3-hydroxycyclopentyl]-2-(2-methylprop-2-yl)pyrazol-3-yl}amino)-4-methyl-3-oxo-3,4-dihydro-2H-1λ6-benzo[2,1-e][1,2,4]thiadiazine-1,1-dione A solution of 4-methyl-6-{[2-(2-methylprop-2-yl)-5-[(1S,3R)-3-{[(2-methylprop-2-yl)diphenylsilyl]oxy}cyclopentyl]pyrazol-3-yl]amino}-3-oxo-3,4-dihydro-2H-1λ6-benzo[2,1-e][1,2,4]thiadiazine-1,1-dione (300 mg, 0.45 mmol) in HCOOH (4 mL) was stirred at room temperature for 18 hr. The reaction was concentrated and the residue was dissolved in 10 ml THF and H₂O (1:1). LiOH was added and stirred for another 30 min. The reaction was adjusted pH to 5-6 with 1N HCl, extracted with EA. The combined organic layer was separated, washed with brine, dried over Na₂SO₄, filtered and concentrated. The residue was purified by silica gel column chromatography eluted with methanol in dichloroform (gradient: 0-10%) to afford the title compound 6-({5-[(1S,3R)-3-hydroxycyclopentyl]-2-(2-methylprop-2-yl)pyrazol-3-yl}amino)-4-methyl-3-oxo-3,4-dihydro-2H-16-benzo[2,1-e][1,2,4]thiadiazine-1,1-dione (180 mg, 0.4 mmol, 93%) as a gray solid. LCMS m/z:434 [M+H]⁺.

Step E: (1R,3S)-3-[1-(2-methylprop-2-yl)-5-[(4-methyl-1,1,3-trioxo-3,4-dihydro-2H-1λ6-benzo[2,1-e][1,2,4]thiadiazin-6-yl)amino]pyrazol-3-yl]cyclopentyl [(4-nitrophenyl)oxy]methanoate To a solution of 6-({5-[(1S,3R)-3-hydroxycyclopentyl]-2-(2-methylprop-2-yl)pyrazol-3-yl}amino)-4-methyl-3-oxo-3,4-dihydro-2H-1λ6-benzo[2,1-e][1,2,4]thiadiazine-1,1-dione (200 mg, 0.46 mmol) and DMAP (6 mg, 0.05 mmol) in pyridine (4 mL) were added 4-Nitrophenyl chloroformate (139 mg, 0.7 mmol), and the reaction was stirred at 60° C. for 18 hr. TLC and LCMS indicated the reaction was over. The cooled reaction mixture was concentrated. The residue was dissolved in EA, washed with brine, dried over Na₂SO₄, filtered and concentrated, the residue was purified by silica gel column chromatography eluted with methanol in dichloroform (gradient: 0-10%) to afford the title compound (1R,3S)-3-[1-(2-methylprop-2-yl)-5-[(4-methyl-1,1,3-trioxo-3,4-dihydro-2H-1λ6-benzo[2,1-e][1,2,4]thiadiazin-6-yl)amino]pyrazol-3-yl]cyclopentyl [(4-nitrophenyl)oxy]methanoate (80 mg, 0.13 mmol, 29%) as a gray solid. LCMS m/z: 599 [M+H]⁺.

Step F: (1R,3S)-3-{5-[(4-methyl-1,1,3-trioxo-3,4-dihydro-2H-1λ6-benzo[2,1-e][1,2,4]thiadiazin-6-yl)amino]-1H-pyrazol-3-yl}cyclopentyl[(4-nitrophenyl)oxy]methanoate A solution of (1R,3S)-3-[1-(2-methylprop-2-yl)-5-[(4-methyl-1,1,3-trioxo-3,4-dihydro-2H-6-benzo[2,1-e][1,2,4]thiadiazin-6-yl)amino]pyrazol-3-yl]cyclopentyl [(4-nitrophenyl)oxy]methanoate (80 mg, 0.13 mmol) in HCOOH (5 mL) was stirred at 100° C. at N₂ atmosphere for 16 h. LCMS showed ok. The reaction solution was concentrated to give (1R,3S)-3-{5-[(4-methyl-1,1,3-trioxo-3,4-dihydro-2H-16-benzo[2,1-e][1,2,4]thiadiazin-6-yl)amino]-1H-pyrazol-3-yl}cyclopentyl [(4-nitrophenyl)oxy]methanoate (60 mg, 0.11 mmol, 83%) as yellow solid which was used directly for next step without purification. LCMS m/z:543 [M+H]⁺.

Step G: (1R,3S)-3-{5-[(4-methyl-1,1,3-trioxo-3,4-dihydro-2H-1λ6-benzo[2,1-e][1,2,4]thiadiazin-6-yl)amino]-1H-pyrazol-3-yl}cyclopentyl (prop-2-ylamino)methanoate To a solution of (1R,3S)-3-{5-[(4-methyl-1,1,3-trioxo-3,4-dihydro-2H-6-benzo[2,1-e][1,2,4]thiadiazin-6-yl)amino]-1H-pyrazol-3-yl}cyclopentyl [(4-nitrophenyl)oxy]methanoate (100 mg, 0.18 mmol) in THF (3 mL) was added isopropylamine (3 mL). The reaction was stirred at room temperature for 1 hr. The reaction mixture was concentrated and the residue was purified by prep-HPLC to afford the title compound (1R,3S)-3-{5-[(4-methyl-1,1,3-trioxo-3,4-dihydro-2H-1λ6-benzo[2,1-e][1,2,4]thiadiazin-6-yl)amino]-1H-pyrazol-3-yl}cyclopentyl (prop-2-ylamino)methanoate (10.4 mg, 0.02 mmol, 12%) as an off-white solid. LCMS m/z: 463 [M+H]⁺. ¹H NMR (400 MHz, DMSO) δ 11.99 (s, 1H), 8.91 (s, 1H), 7.49 (d, J=8.4 Hz, 1H), 7.41 (s, 1H), 7.31-6.92 (m, 4H), 5.74 (s, 1H), 5.05 (s, 1H), 3.64 (d, J=6.4 Hz, 1H), 3.33 (s, 3H), 3.15-3.05 (m, 1H), 2.52-2.44 (m, 1H), 2.10-2.05 (m, 1H), 2.01-1.90 (m, 1H), 1.80-1.65 (m, 3H), 1.09 (d, J=6.4 Hz, 6H).

### Example 19: (1R,3S)-3-(3-((1,1-dioxido-2,3-dihydrobenzo[b]thiophen-6-yl)amino)-1H-pyrazol-5-yl)cyclopentyl isopropylcarbamate

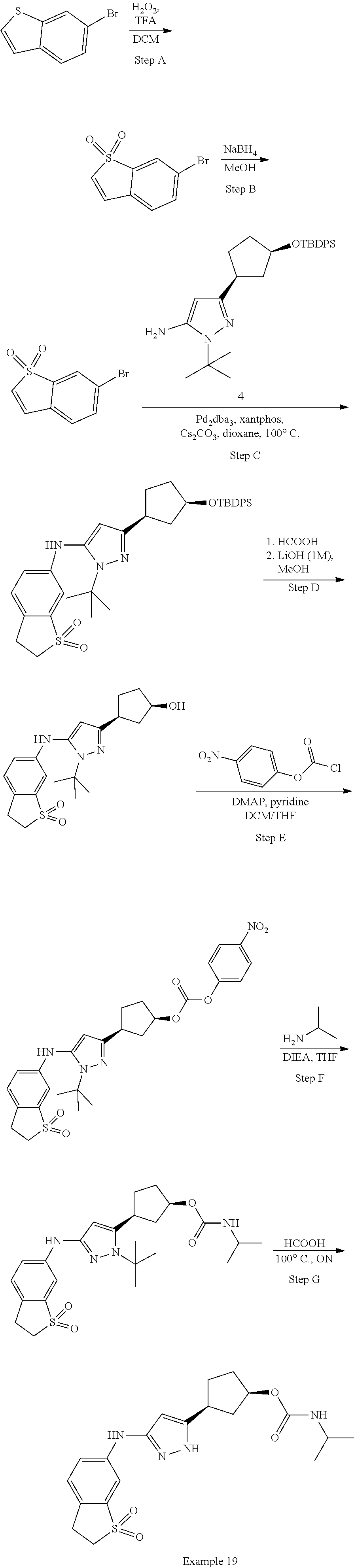

Step A: 6-bromobenzo[b]thiophene 1,1-dioxide To a solution of 6-bromobenzothiophene (2.0 g, 9.39 mmol) in DCM (15 mL) was added H₂O₂(4 mL, 39.2 mmol, 30% in H₂O) and TFA (4 mL, 53.8 mmol). The mixture was stirred at 20° C. for 5 hours. The mixture was purified by silica gel chromatography (20 g column) using 0-30% EtOAc/hexane. The product-containing fractions were concentrated to afford 6-bromobenzo[b]thiophene 1,1-dioxide (2.1 g, 8.57 mmol, 91%) as a white solid.

Step B: 6-bromo-2,3-dihydrobenzo[b]thiophene 1,1-dioxide To a solution of 6-bromo-1λ⁶-benzothiophene-1,1-dione (1.0 g, 4.08 mmol) in MeOH (30 mL) was added NaBH₄ (300 mg, 8.98 mmol) and the mixture was stirred at 20° C. for 2 hours and then concentrated. The residue was purified by silica gel chromatography (10 g column) using 0-40% EtOAc/hexane. The product-containing fractions were concentrated to afford 6-bromo-2,3-dihydrobenzo[b]thiophene 1,1-dioxide (900 mg, 3.64 mmol, 89%) as a white solid.

Step C: 6-((1-(tert-butyl)-3-((1S,3R)-3-((tert-butyldiphenylsilyl)oxy)cyclopentyl)-1H-pyrazol-5-yl)amino)-2,3-dihydrobenzo[b]thiophene 1,1-dioxide To a solution of 6-bromo-2,3-dihydro-1λ⁶-benzothiophene-1,1-dione (300 mg, 1.214 mmol) and 2-(2-methylprop-2-yl)-5-[(1S,3R)-3-{[(2-methylprop-2-yl)diphenylsilyl]oxy}cyclopentyl]pyrazol-3-amine (616 mg, 1.34 mmol) in 1,4-dioxane (8 mL) was added Cs₂CO₃ (791 mg, 2.43 mmol). Then Pd₂(dba)₃ (111 mg, 0.121 mmol) and Xantphos (140 mg, 0.24 mmol) was added into the mixture and the mixture was stirred at 100° C. under N₂ for 5 hours. The mixture was purified by silica gel chromatography (2 g column) using 0-50% EtOAc/hexane. The product-containing fractions were concentrated to afford 6-((1-(tert-butyl)-3-((1S,3R)-3-((tert-butyldiphenylsilyl)oxy)cyclopentyl)-1H-pyrazol-5-yl)amino)-2,3-dihydrobenzo[b]thiophene 1,1-dioxide (530 mg, 0.84 mmol, 69%) as a brown solid. LCMS: ESI m/z 628.4 [M+H]⁺

Step D: 6-((1-(tert-butyl)-3-((1S,3R)-3-hydroxycyclopentyl)-1H-pyrazol-5-yl)amino)-2,3-dihydrobenzo[b]thiophene 1,1-dioxide A solution of 6-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1λ⁶-benzothiophene-1,1-dione (520 mg, 0.83 mmol) in formic acid (4 mL) was stirred at 20° C. for 30 min. The mixture was concentrated under vacuum to remove formic acid to give a crude, which was added MeOH (3 mL) and LiOH (100 mg) was stirred at 20° C. for 30 min. The reaction mixture was concentrated under vacuum and poured into EA (30 mL), extracted with water. The organic layer was concentrated to give 6-((1-(tert-butyl)-3-((1S,3R)-3-hydroxycyclopentyl)-1H-pyrazol-5-yl)amino)-2,3-dihydrobenzo[b]thiophene 1,1-dioxide (270 mg, 0.69 mmol, 84%) as a brown solid. LCMS: ESI m/z 390.2 [M+H]⁺

Step E: (1R,3S)-3-(1-(tert-butyl)-5-((1,1-dioxido-2,3-dihydrobenzo[b]thiophen-6-yl)amino)-1H-pyrazol-3-yl)cyclopentyl (4-nitrophenyl) carbonate To a solution of 6-({5-[(1S,3R)-3-hydroxycyclopentyl]-2-(2-methylprop-2-yl)pyrazol-3-yl}amino)-2,3-dihydro-1λ⁶-benzothiophene-1,1-dione (270 mg, 0.69 mmol) in DCM (3 mL) and THF (3 mL) was added 4-nitrophenyl chloromethanoate (210 mg, 1.04 mmol). Then pyridine (0.11 mL, 1.39 mmol) and DMAP (17 mg, 0.14 mmol) was added. The reaction mixture was stirred at 20° C. for 1 hours. The mixture was purified by silica gel chromatography (2 g column) using 0-50% EtOAc/hexane. The product-containing fractions were concentrated to afford (1R,3S)-3-(1-(tert-butyl)-5-((1,1-dioxido-2,3-dihydrobenzo[b]thiophen-6-yl)amino)-1H-pyrazol-3-yl)cyclopentyl (4-nitrophenyl) carbonate (160 mg, 0.29 mmol, 42%) as a brown solid. LCMS: ESI m/z 555.2 [M+H]⁺

Step F: (1R,3S)-3-(1-(tert-butyl)-3-((1,1-dioxido-2,3-dihydrobenzo[b]thiophen-6-yl)amino)-1H-pyrazol-5-yl)cyclopentyl isopropylcarbamate To a solution of (1R,3S)-3-{5-[(1,1-dioxo-2,3-dihydro-1λ⁶-benzo[b]thiophen-6-yl)amino]-1-(2-methylprop-2-yl)pyrazol-3-yl}cyclopentyl [(4-nitrophenyl)oxy]methanoate (100 mg, 0.18 mmol) in THF (5 mL) was added DIEA (0.045 mL, 0.27 mmol). Then propan-2-amine (0.09 mL, 1.10 mmol) was added into the mixture dropwise and the mixture was stirred at 20° C. for 2 hours and then concentrated. The residue was purified by silica gel chromatography to give (1R,3S)-3-(1-(tert-butyl)-3-((1,1-dioxido-2,3-dihydrobenzo[b]thiophen-6-yl)amino)-1H-pyrazol-5-yl)cyclopentyl isopropylcarbamate (70 mg, 0.15 mmol, 82%) as a yellow oil. LCMS: ESI m/z 475.2 [M+H]⁺

Step G: (1R,3S)-3-(3-((1,1-dioxido-2,3-dihydrobenzo[b]thiophen-6-yl)amino)-1H-pyrazol-5-yl)cyclopentyl isopropylcarbamate A slution of (1R,3S)-3-{5-[(1,1-dioxo-2,3-dihydro-1λ⁶-benzo[b]thiophen-6-yl)amino]-2-(2-methylprop-2-yl)pyrazol-3-yl}cyclopentyl (prop-2-ylamino)methanoate (70 mg, 0.15 mmol) in formic acid (4 mL) was stirred at 100° C. for 12 hours. The reaction mixture was concentrated and the residue was purified by prep-HPLC to give (1R,3S)-3-(3-((1,1-dioxido-2,3-dihydrobenzo[b]thiophen-6-yl)amino)-1H-pyrazol-5-yl)cyclopentyl isopropylcarbamate (16.3 mg, 0.04 mmol, 26%) as a white solid. LCMS: ESI m/z 419.2 [M+H]⁺. ¹H NMR (400 MHz, DMSO) δ 11.81 (s, 1H), 8.81 (s, 1H), 7.94 (s, 1H), 7.38 (d, J=8.4 Hz, 1H), 7.28 (d, J=8.4 Hz, 1H), 6.95 (d, J=9.6 Hz, 1H), 5.61 (s, 1H), 5.07-4.93 (m, 1H), 3.64-3.55 (m, 1H), 3.52 (t, J=6.8 Hz, 2H), 3.20 (t, J=6.8 Hz, 2H), 3.12-2.99 (m, 1H), 2.48-2.40 (m, 1H), 2.07-1.97 (m, 1H), 1.97-1.84 (m, 1H), 1.80-1.66 (m, 2H), 1.66-1.53 (m, 1H), 1.03 (d, J=6.4 Hz, 6H).

### Example 20: (1R,3S)-3-{5-[(3-oxo-2,3-dihydro-1H-isoindol-5-yl)amino]-2H-pyrazol-3-yl}cyclopentyl (prop-2-ylamino)methanoate

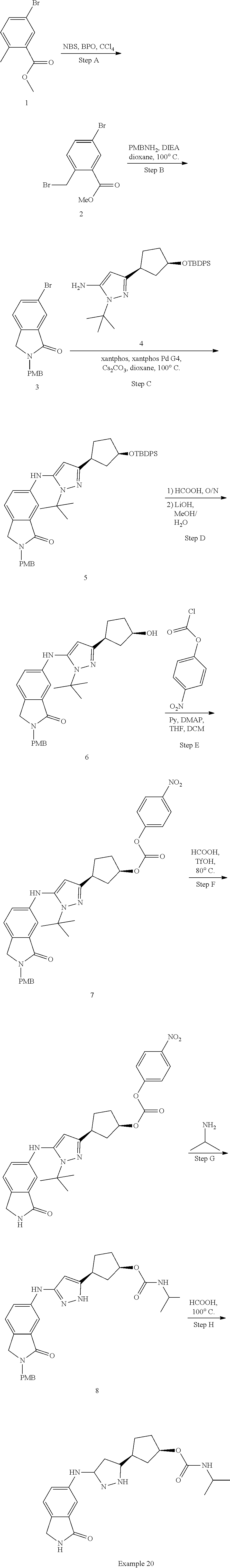

Step A: 5-bromo-2-(bromomethyl)benzoate To a solution of methyl 5-bromo-2-methylbenzoate (3 g, 13.1 mmol) in CCl₄ (30 mL) was added NBS (3.50 g, 19.6 mmol) and BPO (1.59 g, 6.55 mmol). The reaction mixture was stirred at 80° C. for 6 h. The cooled reaction mixture was poured into water and extracted with EtOAc. The combined organic phase was washed with brine, dried with Na₂SO₄, filtered and concentrated. The residue was purified by flash column chromatography with 0-10% ethyl acetate in petroleum ether to afford crude methyl 5-bromo-2-(bromomethyl)benzoate (3 g, 9.74 mmol, 74.4%) as a white solid. Step B: 6-bromo-2-[(4-methoxyphenyl)methyl]-2,3-dihydro-1H-isoindol-1-one

To a solution of methyl 5-bromo-2-(bromomethyl)benzoate (3 g, 9.74 mmol) in THF (30 mL) was added (4-methoxyphenyl)methanamine (1.40 mL, 10.7 mmol). The reaction mixture was stirred at 80° C. for 2 h. The cooled reaction mixture was poured into water and extracted with EtOAc. The combined organic phase was washed with brine, dried with Na₂SO₄, filtered and concentrated. The residue was purified by flash column chromatography with 0-50% ethyl acetate in petroleum ether to afford 6-bromo-2-[(4-methoxyphenyl)methyl]-2,3-dihydro-1H-isoindol-1-one (2 g, 6.02 mmol, 61.8%) as a white solid. LCMS: ESI m/z 332 [M+H]⁺

Step C: 2-[(4-methoxyphenyl)methyl]-6-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1H-isoindol-1-one To a solution of 6-bromo-2-[(4-methoxyphenyl)methyl]-2,3-dihydro-1H-isoindol-1-one (258 mg, 0.779 mmol) in dioxane (10 mL) was added 2-(2-methylprop-2-yl)-5-[(1S,3R)-3-{[(2-methylprop-2-yl)diphenylsilyl]oxy}cyclopentyl]pyrazol-3-amine (300 mg, 0.650 mmol), Cs₂CO₃ (529 mg, 1.62 mmol), xantphos (75.2 mg, 0.130 mmol) and XantPhos Pd G₄ (62.6 mg, 0.065 mmol), and the reaction was purged with N₂ and stirred at 100° C. under N₂ overnight. The cooled reaction mixture was diluted with EA and water. The organic layer was separated, washed with further brine, and concentrated in vacuo. The residue was purified using silica gel column chromatography eluting with ethyl acetate in petroleum ether (2/1) The organic layer was collected, concentrated in vacuo, and dried to afford the title compound 2-[(4-methoxyphenyl)methyl]-6-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1H-isoindol-1-one (420 mg, 0.589 mmol, 90.7%) as a yellow oil. LCMS: ESI m/z 713 [M+H]⁺.

Step D: 6-({5-[(1S,3R)-3-hydroxycyclopentyl]-2-(2-methylprop-2-yl)pyrazol-3-yl}amino)-2-[(4-methoxyphenyl)methyl]-2,3-dihydro-1H-isoindol-1-one A mixture of 2-[(4-methoxyphenyl)methyl]-6-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1H-isoindol-1-one (420 mg, 0.589 mmol) in HCOOH (6 mL) was stirred at RT overnight and then concentrated under reduced pressure. The residue was treated with a mixture of LiOH—H₂O (0.065 mL, 2.36 mmol) in MeOH (3 mL)/H₂O (3 mL). The resulting mixture was stirred at rt for 1 h and then concentrated under reduced pressure. The following mixture was poured into water and extracted with EtOAc. The combined organic phase was washed with brine, dried with Na₂SO₄, filtered and concentrated. The residue was purified by chromatography (silica gel, 0-70%, EA in PE) to give 6-({5-[(1S,3R)-3-hydroxycyclopentyl]-2-(2-methylprop-2-yl)pyrazol-3-yl}amino)-2-[(4-methoxyphenyl)methyl]-2,3-dihydro-1H-isoindol-1-one (210 mg, 0.442 mmol, 75.1%) as a white solid. LCMS: ESI m/z 475 [M+H]⁺.

Step E: (1R,3S)-3-[5-({2-[(4-methoxyphenyl)methyl]-3-oxo-2,3-dihydro-1H-isoindol-5-yl}amino)-1-(2-methylprop-2-yl)pyrazol-3-yl]cyclopentyl [(4-nitrophenyl)oxy]methanoate To a solution of 6-({5-[(1S,3R)-3-hydroxycyclopentyl]-2-(2-methylprop-2-yl)pyrazol-3-yl}amino)-2-[(4-methoxyphenyl)methyl]-2,3-dihydro-1H-isoindol-1-one (210 mg, 0.442 mmol) in THF (2.5 mL) and DCM (2.5 mL) were added 4-nitrophenyl chloromethanoate (267 mg, 1.33 mmol), DMAP (5.40 mg, 0.044 mmol) and Py (0.107 mL, 1.326 mmol), the reaction was stirred at rt for 2 h. The reaction was concentrated in vacuo. The residue was purified using silica gel column chromatography eluting with EA in PE [gradient: 0-50%]. to afford the title compound (1R,3S)-3-[5-({2-[(4-methoxyphenyl)methyl]-3-oxo-2,3-dihydro-1H-isoindol-5-yl}amino)-1-(2-methylprop-2-yl)pyrazol-3-yl]cyclopentyl [(4-nitrophenyl)oxy]methanoate (160 mg, 0.250 mmol, 56.5%) as a white solid. LCMS: ESI 640[M+H]⁺

Step F: (1R,3S)-3-[1-(2-methylprop-2-yl)-5-[(3-oxo-2,3-dihydro-1H-isoindol-5-yl)amino]pyrazol-3-yl]cyclopentyl [(4-nitrophenyl)oxy]methanoate A solution of (1R,3S)-3-[5-({2-[(4-methoxyphenyl)methyl]-3-oxo-2,3-dihydro-1H-isoindol-5-yl}amino)-1-(2-methylprop-2-yl)pyrazol-3-yl]cyclopentyl [(4-nitrophenyl)oxy]methanoate (60 mg, 0.09 mmol) in HCOOH (4 mL) and TfOH (0.4 mL) was stirred at 80° C. at N₂ atmosphere for 2 hours. LCMS showed 50% desire product was found. The reaction was concentrated to give a crude desire product (1R,3S)-3-[1-(2-methylprop-2-yl)-5-[(3-oxo-2,3-dihydro-1H-isoindol-5-yl)amino]pyrazol-3-yl]cyclopentyl [(4-nitrophenyl)oxy]methanoate (60 mg, 0.08 mmol, -50% purity, yield 61%) as a gray solid which was used directly for next step without purification. LCMS: ESI 520 [M+H]⁺

Step G: (1R,3S)-3-[1-(2-methylprop-2-yl)-5-[(3-oxo-2,3-dihydro-1H-isoindol-5-yl)amino]pyrazol-3-yl]cyclopentyl (prop-2-ylamino)methanoate To a solution of ((1R,3S)-3-[1-(2-methylprop-2-yl)-5-[(3-oxo-2,3-dihydro-1H-isoindol-5-yl)amino]pyrazol-3-yl]cyclopentyl [(4-nitrophenyl)oxy]methanoate (80 mg, 0.125 mmol) in THF (3 mL) was added isopropylamine (3 mL). The reaction solution was stirred at room temperature for 1 h. LCMS showed the reaction was over. The reaction mixture was concentrated and purified by silica gel column chromatography eluted with EA in PE [gradient: 0-100%] to give (1R,3S)-3-[1-(2-methylprop-2-yl)-5-[(3-oxo-2,3-dihydro-1H-isoindol-5-yl)amino]pyrazol-3-yl]cyclopentyl (prop-2-ylamino)methanoate (40 mg, 0.09 mmol, 72.8%) as a yellow solid. LCMS: ESI 440 [M+H]⁺

Step H: (1R,3S)-3-{5-[(3-oxo-2,3-dihydro-1H-isoindol-5-yl)amino]-2H-pyrazol-3-yl}cyclopentyl (prop-2-ylamino)methanoate A solution of (1R,3S)-3-[1-(2-methylprop-2-yl)-5-[(3-oxo-2,3-dihydro-1H-isoindol-5-yl)amino]pyrazol-3-yl]cyclopentyl (prop-2-ylamino)methanoate (40 mg, 0.09 mmol) in HCOOH (4 mL) was stirred at 100° C. for 16 h. The cooled reaction mixture was concentrated. The residue was purified by prep-HPLC to afford the title compound (1R,3S)-3-{5-[(3-oxo-2,3-dihydro-1H-isoindol-5-yl)amino]-2H-pyrazol-3-yl}cyclopentyl (prop-2-ylamino)methanoate (1.3 mg, 0.003 mmol, 3.7%) as a white solid. LCMS: ESI 384[M+H]⁺. ¹HNMR (400 MHz, DMSO) δ 11.73 (s, 1H), 8.50 (s, 1H), 8.36 (s, 1H), 7.79 (s, 1H), 7.42 (d, J=8.2 Hz, 1H), 7.33 (d, J=8.2 Hz, 1H), 6.94 (s, 1H), 5.63 (s, 1H), 5.01 (s, 1H), 4.24 (s, 2H), 3.63-3.55 (m, 1H), 3.11-3.03 (m, 1H), 2.51-2.43 (m, 1H), 2.08-1.97 (m, 1H), 1.97-1.84 (m, 1H), 1.84-1.61 (m, 3H), 1.04 (d, J=6.4 Hz, 6H).

### Example 21: (1R,3S)-3-{5-[(3,3-dimethyl-1,1-dioxo-2,3-dihydro-1λ⁶-benzothiophen-5-yl)amino]-2H-pyrazol-3-yl}cyclopentyl (prop-2-ylamino)methanoate

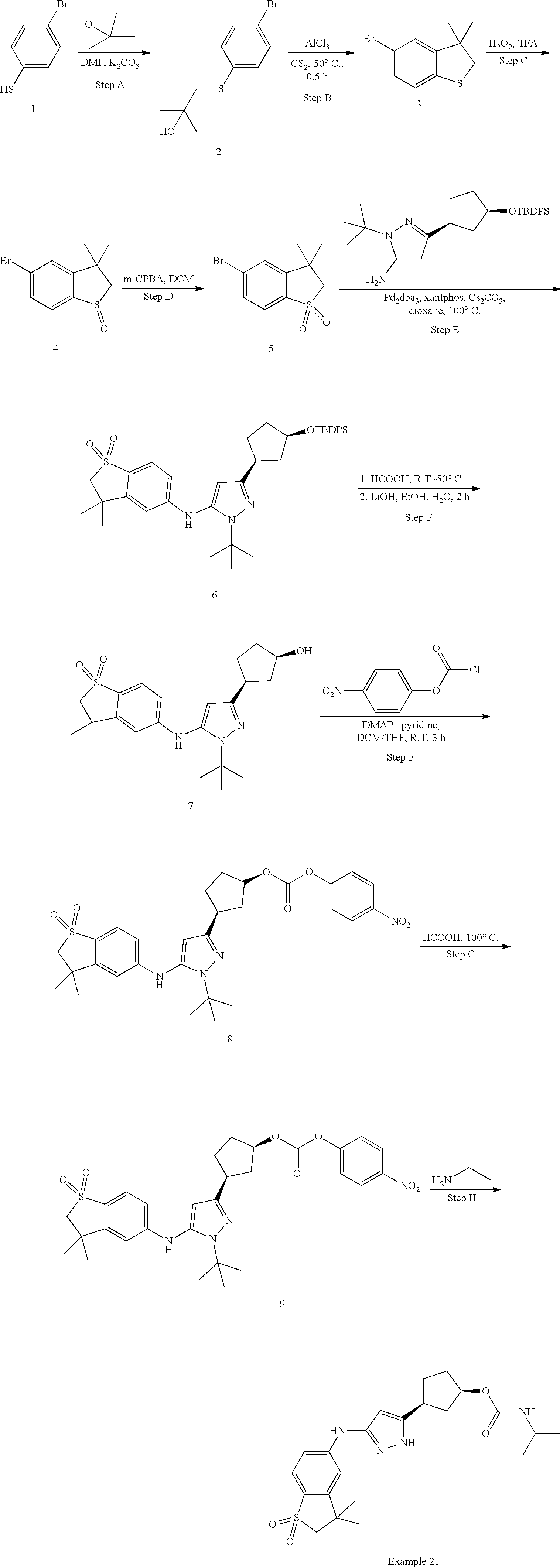

Step A: 1-[(4-bromophenyl)sulfanyl]-2-methylpropan-2-ol To a solution of compound 4-bromobenzene-1-thiol (10 g, 52.9 mmol) in DMF (100 mL) was added 2,2-dimethyloxirane (5.19 mL, 58.2 mmol) and K₂CO₃ (10.9 g, 79.3 mmol). The resulting reaction solution was stirred at 20° C. for 0.5 hours. The reaction was quenched by the addition of saturated sodium chloride solution (500 mL) and then extracted with ethyl acetate (300 mL×3). The organic phases were combined and washed with saturated sodium chloride solution (100 mL×3), dried over anhydrous sodium sulfate, filtered, and concentrated under reduced pressure to give a residue. The residue was purified by column chromatography (petroleum ether/ethyl acetate=100-0%) to give compound 1-[(4-bromophenyl)sulfanyl]-2-methylpropan-2-ol (10 g, 38.288 mmol, 72.39%) as a colorless oil. ¹H NMR (400 MHz, DMSO) δ 7.46 (d, J=8.4 Hz, 2H), 7.30 (d, J=8.4 Hz, 2H), 4.68 (s, 1H), 3.04 (s, 2H), 1.19 (s, 6H).

Step B: 5-bromo-3,3-dimethyl-2,3-dihydrobenzothiophene To a suspension of aluminum chloride (7.66 g, 57.432 mmol) in carbon disulfide (100 mL) was added a solution of 1-[(4-bromophenyl)sulfanyl]-2-methylpropan-2-ol (5 g, 19.144 mmol) in 100 mL of a carbon disulfide solution at −10° C. The resulting reaction solution was stirred at 50° C. for 0.5 hours and then cooled to 0° C. 1N Dilute hydrochloric acid was added to the reaction system, followed by extraction with ethyl acetate. The organic phases were combined and washed with saturated sodium chloride solution, dried over anhydrous sodium sulfate, filtered, and concentrated under reduced pressure to give a residue. The residue was separated and purified by column chromatography (petroleum ether/ethyl acetate=100-0%) to give compound 5-bromo-3,3-dimethyl-2,3-dihydrobenzothiophene (1.2 g, 4.935 mmol, 25.78%) as a yellow oil. ¹H NMR (400 MHz, CDCl₃) δ 7.24 (dd, J=8.0, 2.0 Hz, 1H), 7.16 (d, J=2.0 Hz, 1H), 7.06 (d, J=8.0 Hz, 1H), 3.20 (s, 2H), 1.39 (d, J=4.4 Hz, 6H).

Step C: 5-bromo-3,3-dimethyl-2,3-dihydro-1%⁴-benzothiophen-1-one To a solution of 5-bromo-3,3-dimethyl-2,3-dihydrobenzothiophene (1 g, 4.1 mmol) in 1,2-dichloroethane (10 mL) was added slowly acetic acid (0.825 mL, 14.4 mmol) and H₂O₂(1 mL, 30 wt % in H₂O, 9.8 mmol). The reaction mixture was stirred at 40° C. for 3.5 hours. Ice water was added to the reaction mixture, and then Na₂S₂O₃ (aqueous) was added. The following mixture was extracted with ethyl acetate, washed with a sodium carbonate aqueous solution twice, washed with a saturated sodium chloride aqueous solution, dried over anhydrous sodium sulfate and concentrated. The residue was purified by flash chromatography to 5-bromo-3,3-dimethyl-2,3-dihydro-1λ⁴-benzothiophen-1-one (500 mg, 1.929 mmol, 46.91%) as a white solid. LCMS: ESI m/z 259 [M+H]⁺.

Step D: 5-bromo-3,3-dimethyl-2,3-dihydro-1)⁶-benzothiophene-1,1-dione To a solution of 5-bromo-3,3-dimethyl-2,3-dihydro-1λ⁴-benzothiophen-1-one (500 mg, 1.929 mmol) in DCM (10 mL) was added 85% 3-chlorobenzene-1-carboperoxoic acid (832 mg, 4.82 mmol) at 0° C. The reaction mixture was stirred at 5-15° C. for 3 hr. The reaction mixture purified by silica gel column chromatograph to afford the title compound 5-bromo-3,3-dimethyl-2,3-dihydro-1λ⁶-benzothiophene-1,1-dione (300 mg, 1.09 mmol, 56.5%) as a white solid. LCMS: ESI m/z 275 [M+H]⁺.

Step E: 3,3-dimethyl-5-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1λ⁶-benzothiophene-1,1-dione To a solution of 5-bromo-3,3-dimethyl-2,3-dihydro-1λ⁶-benzothiophene-1,1-dione (300 mg, 1.090 mmol) in dioxane (10 mL) were added 2-(2-methylprop-2-yl)-5-[(1S,3R)-3-{[(2-methylprop-2-yl)diphenylsilyl]oxy}cyclopentyl]pyrazol-3-amine (503 mg, 1.09 mmol), Cs₂CO₃ (710.47 mg, 2.18 mmol), Xant-PHOS (126 mg, 0.218 mmol) and Pd₂(dba)₃ (99.8 mg, 0.109 mmol). The reaction mixture was stirred at 100° C. under N₂ overnight. The cooled reaction mixture was diluted with EA and water. The organic layer was separated, washed with brine, and concentrated in vacuo. The residue was purified using silica gel column chromatography eluting with ethyl acetate in petroleum ether to afford the title compound 3,3-dimethyl-5-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1⁶-benzothiophene-1,1-dione (500 mg, 0.762 mmol, 69.9%) as a yellow oil. LCMS: ESI m/z 656 [M+H]⁺.

Step F: 5-({5-[(1S,3R)-3-hydroxycyclopentyl]-2-(2-methylprop-2-yl)pyrazol-3-yl}amino)-3,3-dimethyl-2,3-dihydro-1λ⁶-benzothiophene-1,1-dione A mixture of 3,3-dimethyl-5-{[2-(2-methylprop-2-yl)-5-[(1S,3R)-3-{[(2-methylprop-2-yl)diphenylsilyl]oxy}cyclopentyl]pyrazol-3-yl]amino}-2,3-dihydro-1λ⁶-benzothiophene-1,1-dione (500 mg, 0.762 mmol) in formic acid (3 mL) was stirred at RT for 1 h and then concentrated under reduced pressure. The residue was treated with a mixture of lithium hydroxide hydrate (31.98 mg, 0.762 mmol) in MeOH (3.00 mL)/H₂O (0.30 mL). The resulting mixture was stirred at rt for 2 h, the mixture was concentrated under reduced pressure. The mixture was poured into water and extracted with EtOAc. The combined organic phase was washed with brine, dried with Na₂SO₄, filtered and concentrated. The residue was purified by chromatography to give 5-({5-[(1S,3R)-3-hydroxycyclopentyl]-2-(2-methylprop-2-yl)pyrazol-3-yl}amino)-3,3-dimethyl-2,3-dihydro-1λ⁶-benzothiophene-1,1-dione (280 mg, 0.671 mmol, 87.97%) as a colorless oil. LCMS: ESI m/z 418 [M+H]⁺.

Step G: (1R,3S)-3-{5-[(3,3-dimethyl-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate To a solution of 5-({5-[(1S,3R)-3-hydroxycyclopentyl]-2-(2-methylprop-2-yl)pyrazol-3-yl}amino)-3,3-dimethyl-2,3-dihydro-1λ⁶-benzothiophene-1,1-dione (300 mg, 0.718 mmol) in DCM (5 mL) and THF (5 mL) were added 4-nitrophenyl chloromethanoate (289 mg, 1.44 mmol), Py (0.174 mL, 2.15 mmol) and DMAP (8.78 mg, 0.072 mmol). The reaction mixture was stirred under at 25° C. for 2 hr. The reaction solution was diluted with EA, washed with water and brine, dried over anhydrous Na₂SO₄, concentrated in vacuum and the residue was purified by flash chromatography to afford (1R,3S)-3-{5-[(3,3-dimethyl-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate (350 mg, 0.601 mmol, 83.6%) as a yellow solid. LCMS: ESI m/z 583 [M+H]⁺.

Step H: (1R,3S)-3-{5-[(3,3-dimethyl-1,1-dioxo-2,3-dihydro-1λ⁶-benzothiophen-5-yl)amino]-1H-pyrazol-3-yl}cyclopentyl [(4-nitrophenyl)oxy]methanoate To a solution of (1R,3S)-3-{5-[(3,3-dimethyl-1,1-dioxo-2,3-dihydro-1λ⁶-benzothiophen-5-yl)amino]-1-(2-methylprop-2-yl)pyrazol-3-yl}cyclopentyl [(4-nitrophenyl)oxy]methanoate (350 mg, 0.601 mmol) in formic acid (5 mL) at 25° C. The reaction mixture was stirred at 100° C. for overnight. The FA was removed under vacuum to afford (1R,3S)-3-{5-[(3,3-dimethyl-1,1-dioxo-2,3-dihydro-1⁶-benzothiophen-5-yl)amino]-1H-pyrazol-3-yl}cyclopentyl [(4-nitrophenyl)oxy]methanoate (280 mg, 0.532 mmol, 88.5%) as a yellow oil. LCMS: ESI m/z 527 [M+H]⁺.

### Step I: (1R,3S)-3-{5-[(3,3-dimethyl-1,1-dioxo-2,3-dihydro-1λ⁶-benzothiophen-5-yl)amino]-2H-pyrazol-3-yl}cyclopentyl (prop-2-ylamino)methanoate

To a solution of (1R,3S)-3-{5-[(3,3-dimethyl-1,1-dioxo-2,3-dihydro-1λ⁶-benzothiophen-5-yl)amino]-1H-pyrazol-3-yl}cyclopentyl [(4-nitrophenyl)oxy]methanoate (280 mg, 0.532 mmol) in THF (5 mL) was added propan-2-amine (0.091 mL, 1.064 mmol) and the reaction was stirred at rt for 1 h. The reaction mixture was concentrated in vacuo. The residue was purified by prep-HPLC to afford the title compound (1R,3S)-3-{5-[(3,3-dimethyl-1,1-dioxo-2,3-dihydro-1⁶-benzothiophen-5-yl)amino]-2H-pyrazol-3-yl}cyclopentyl (prop-2-ylamino)methanoate (83.5 mg, 0.187 mmol, 35.2%) as a white solid. LCMS: ESI m/z 447 [M+H]⁺. ¹H NMR (400 MHz, DMSO) δ 9.03 (s, 1H), 7.57 (s, 1H), 7.45 (d, J=8.8 Hz, 1H), 7.31 (dd, J=8.8, 1.6 Hz, 1H), 6.94 (d, J=7.6 Hz, 1H), 5.69 (s, 1H), 5.00 (s, 1H), 3.39 (s, 2H), 3.13-3.02 (m, 1H), 2.51-2.43 (m, 1H), 2.07-1.83 (m, 2H), 1.76-1.54 (m, 3H), 1.42 (s, 6H), 1.03 (d, J=6.4 Hz, 6H).

### Example 22: (1R,3S)-3-(3-((2,2-dioxido-1,3-dihydrobenzo[c]thiophen-5-yl)amino)-1H-pyrazol-5-yl)cyclopentyl isopropylcarbamate

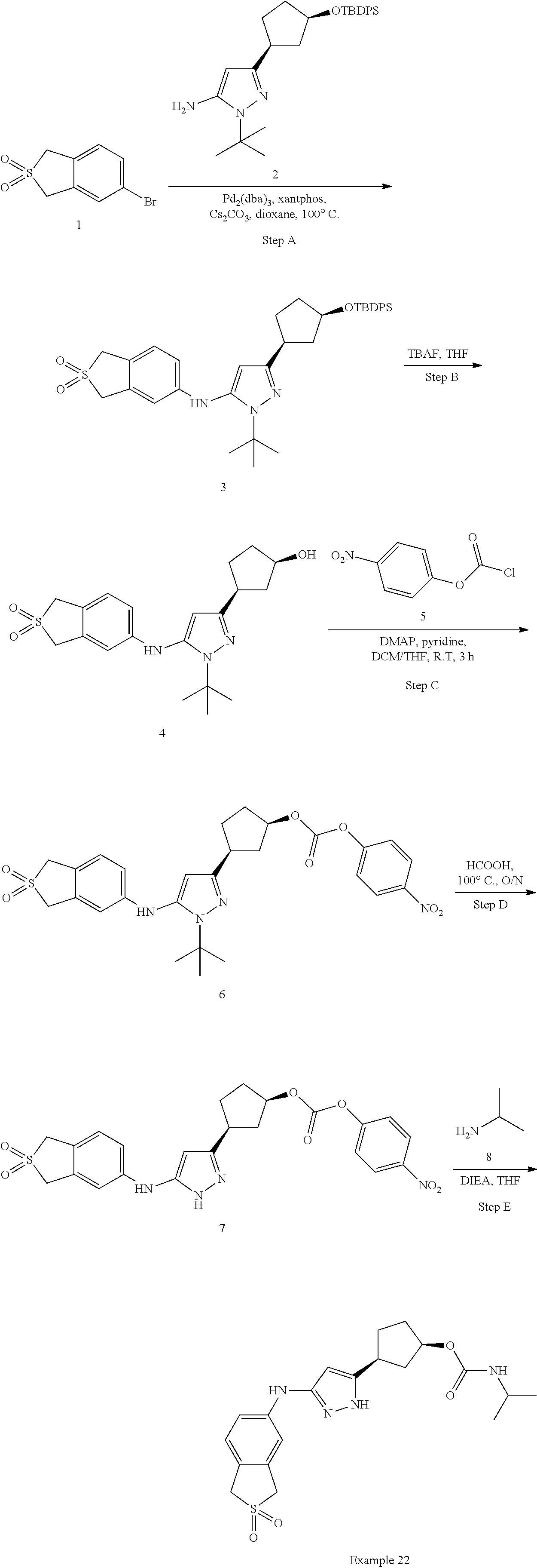

Step A: 5-((1-(tert-butyl)-3-((1S,3R)-3-((tert-butyldiphenylsilyl)oxy)cyclopentyl)-1H-pyrazol-5-yl)amino)-1,3-dihydrobenzo[c]thiophene 2,2-dioxide To a solution of 5-bromo-1,3-dihydrobenzo[c]thiophene 2,2-dioxide (460 mg, 1.86 mmol) in dioxane (15 mL) was added 1-(tert-butyl)-3-((1S,3R)-3-((tert-butyldiphenylsilyl)oxy)cyclopentyl)-1H-pyrazol-5-amine (250 mg, 0.541 mmol), Cs₂CO₃ (529 mg, 1.62 mmol), Pd₂(dba)₃ (49.6 mg, 0.0540 mmol) and Xantphos (62.7 mg, 0.108 mmol). The reaction mixture was stirred at 100° C. for 2 hrs under N₂ atmosphere. The reaction was complete detected by LCMS. The cooled reaction mixture was dissolved in EA (10 mL*3), washed with H₂O (20 mL) and brine, dried over sodium sulfate, filtered, concentrated. The residue was purified by silica gel chromatography (eluting with 0-50% EA in PE) to afford 5-((1-(tert-butyl)-3-((1S,3R)-3-((tert-butyldiphenylsilyl)oxy)cyclopentyl)-1H-pyrazol-5-yl)amino)-1,3-dihydrobenzo[c]thiophene 2,2-dioxide (300 mg, 0.478 mmol, 88.2%) as a white solid. LCMS: ESI m/z 628.92 [M+H]⁺.

Step B: 5-((1-(tert-butyl)-3-((1S,3R)-3-hydroxycyclopentyl)-1H-pyrazol-5-yl)amino)-1,3-dihydrobenzo[c]thiophene 2,2-dioxide A solution of 5-((1-(tert-butyl)-3-((1S,3R)-3-((tert-butyldiphenylsilyl)oxy)cyclopentyl)-1H-pyrazol-5-yl)amino)-1,3-dihydrobenzo[c]thiophene 2,2-dioxide (300 mg, 0.478 mmol) in formic acid (5 mL) was stirred at room temperature for 5 hrs. The reaction was complete detected by LCMS. The solvents were removed under vacuum. Then add EtOH (3 mL), H₂O (3 mL) and Lithium Hydroxide Monohydrate (120 mg, 2.87 mmol) to alkaline pH. The mixture was stirred at room temperature for 1 hr. LCMS showed the reaction was completed. The reaction mixture was dissolved in EA (50 mL), washed with H₂O (10 mL) and brine, dried over sodium sulfate, filtered, concentrated. The residue was purified by silica gel chromatography (eluting with 0-50% EA in PE) to afford 5-((1-(tert-butyl)-3-((1S,3R)-3-hydroxycyclopentyl)-1H-pyrazol-5-yl)amino)-1,3-dihydrobenzo[c]thiophene 2,2-dioxide (260 mg, 0.668 mmol, 69.9%) as a yellow solid. LCMS: ESI m/z 390.51 [M+H]⁺.

Step C: (1R,3S)-3-(1-(tert-butyl)-5-((2,2-dioxido-1,3-dihydrobenzo[c]thiophen-5-yl)amino)-1H-pyrazol-3-yl)cyclopentyl (4-nitrophenyl) carbonate A suspension of 5-((1-(tert-butyl)-3-((1S,3R)-3-hydroxycyclopentyl)-1H-pyrazol-5-yl)amino)-1,3-dihydrobenzo[c]thiophene 2,2-dioxide (120 mg, 0.308 mmol), DMAP (3.76 mg, 0.0310 mmol), Py (0.0750 mL, 0.924 mmol) and 4-nitrophenyl chloromethanoate (310 mg, 1.54 mmol) in DCM (3 mL) and THF (3 mL) was stirred at room temperature for 3 hrs. The solvents were removed under vacuum. The residue was dissolved in EA (50 mLO), washed with H₂O (50 mL) and brine, dried over sodium sulfate, filtered, concentrated. The residue was purified by silica gel chromatography (eluting with 0-50% EA in PE) to afford (1R,3S)-3-(1-(tert-butyl)-5-((2,2-dioxido-1,3-dihydrobenzo[c]thiophen-5-yl)amino)-1H-pyrazol-3-yl)cyclopentyl (4-nitrophenyl) carbonate (120 mg, 0.216 mmol, 70.2%) as a yellow solid. LCMS: ESI m/z 555.62 [M+H]⁺.

Step D: (1R,3S)-3-(5-((2,2-dioxido-1,3-dihydrobenzo[c]thiophen-5-yl)amino)-1H-pyrazol-3-yl)cyclopentyl (4-nitrophenyl) carbonate A solution of (1R,3S)-3-(1-(tert-butyl)-5-((2,2-dioxido-1,3-dihydrobenzo[c]thiophen-5-yl)amino)-1H-pyrazol-3-yl)cyclopentyl (4-nitrophenyl) carbonate (120 mg, 0.216 mmol) in formic acid (2 mL) was stirred at 100° C. overnight. LCMS showed the reaction was completed. The cooled reaction mixture was concentrated to give crude (1R,3S)-3-(5-((2,2-dioxido-1,3-dihydrobenzo[c]thiophen-5-yl)amino)-1H-pyrazol-3-yl)cyclopentyl (4-nitrophenyl) carbonate (105 mg, 0.211 mmol, 97.4%) as a yellow oil. LCMS: ESI m/z 499.51 [M+H]⁺.

Step E: (1R,3S)-3-(3-((2,2-dioxido-1,3-dihydrobenzo[c]thiophen-5-yl)amino)-1H-pyrazol-5-yl)cyclopentyl isopropylcarbamate A suspension of (1R,3S)-3-(5-((2,2-dioxido-1,3-dihydrobenzo[c]thiophen-5-yl)amino)-1H-pyrazol-3-yl)cyclopentyl (4-nitrophenyl) carbonate (105 mg, 0.211 mmol) in THF (2 mL) and propan-2-amine (0.0540 mL, 0.632 mmol) and DIEA (0.104 mL, 0.632 mmol) was stirred at room temperature for 3 hrs. LCMS showed the reaction had been completed. The reaction was dissolved in EA (50 mL), washed with H₂O (10 mL) and brine, dried over sodium sulfate, filtered, concentrated. The residue was purified by silica gel chromatography (eluting with 0-12% MeOH in DCM) and prep-HPLC (C18, 15˜95% MeCN in H₂O with 0.1% NH₄0H) to afford (1R,3S)-3-(3-((2,2-dioxido-1,3-dihydrobenzo[c]thiophen-5-yl)amino)-1H-pyrazol-5-yl)cyclopentyl isopropylcarbamate (21.8 mg, 0.0520 mmol, 24.7%) as a white solid. LCMS: 419.51 [M+H]⁺. ¹H NMR (400 MHz, DMSO) δ 11.74 (s, 1H), 8.48 (s, 1H), 7.42 (s, 1H), 7.23 (d, J=7.6 Hz, 1H), 7.15 (d, J=7.6 Hz, 1H), 6.95 (s, 1H), 5.64 (s, 1H), 5.00 (s, 1H), 4.42 (s, 2H), 4.33 (s, 2H), 3.58 (s, 1H), 3.05 (s, 1H), 2.50-2.42 (m, 1H), 2.06-1.88 (m, 2H), 1.79-1.56 (m, 3H), 1.04 (d, J=6.0 Hz, 6H).

### Example 23: racemic cis 3-(5-((2-methyl-1,1-dioxido-2,3-dihydrobenzo[d]isothiazol-5-yl)amino)-1H-pyrazol-3-yl)cyclopentyl isopropylcarbamate

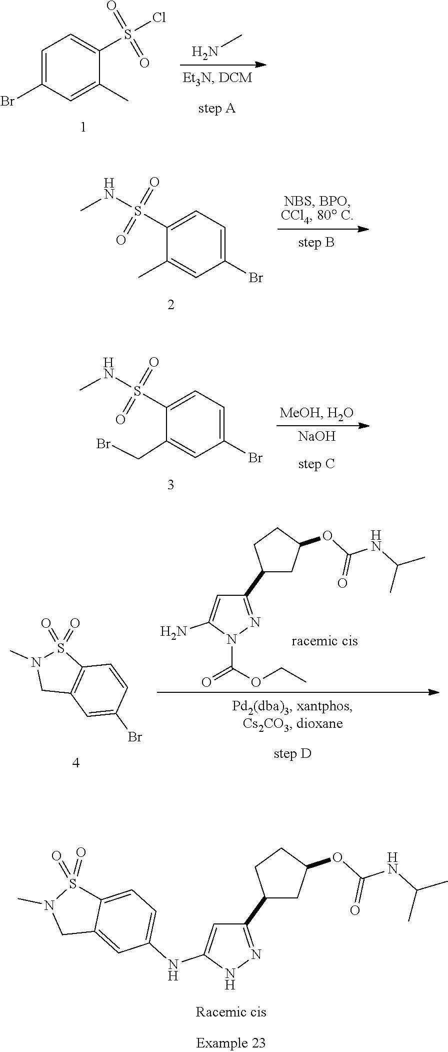

Step A: 4-bromo-2,N-dimethylbenzenesulfonamide To a solution of 4-bromo-2-methylbenzenesulfonyl chloride (5 g, 18.6 mmol) in DCM (20 mL) was added methanamine-THF (5 mL, 10 mmol, 2M in THF) and Et₃N (7.74 mL, 55.6 mmol). The reaction mixture was stirred at room temperature overnight. The reaction mixture was concentrated in vacuo and dissolved in EA (50 mL), washed with H₂O (20 mL) and brine, dried over sodium sulfate and concentrated. The residue was purified by silica gel chromatography (eluting with 0-50% EA in PE) to afford 4-bromo-2,N-dimethylbenzenesulfonamide (2.3 g, 8.71 mmol, 46.9%) as a white solid. LCMS: ESI m/z 266 [M+H]⁺

Step B: 4-bromo-2-(bromomethyl)-N-methylbenzenesulfonamide To a solution of 4-bromo-2,N-dimethylbenzenesulfonamide (1.8 g, 6.82 mmol) in tetrachloromethane (15 mL) was added BPO (0.33 g, 1.36 mmol) and NBS (1.33 g, 7.50 mmol) at rt. The reaction mixture was stirred at 80° C. overnight. The cooled reaction mixture was concentrated. The following mixture was poured into water and extracted with EtOAc. The combined organic phase was washed with brine, dried with Na₂SO₄, filtered and concentrated. The residue was purified using silica gel column chromatography eluting with EA in PE [gradient: 0-30%]. to afford the title compound 4-bromo-2-(bromomethyl)-N-methylbenzenesulfonamide (970 mg, 2.83 mmol, 41.5%) as a white solid. LCMS: ESI 343 [M+H]⁺.

Step C: 5-bromo-2-methyl-2,3-dihydro-1λ\hat{ }6-benzo[2,1-d][1,2]thiazole-1,1-dione To a solution of 4-bromo-2-(bromomethyl)-N-methylbenzenesulfonamide (970 mg, 2.83 mmol) in H₂O (2 mL) and MeOH (8 mL) was added NaOH (226 mg, 5.66 mmol). The reaction mixture was stirred at rt for 3 h. The reaction mixture was concentrated in vacuo. The following mixture was poured into water and extracted with EtOAc. The combined organic phase was washed with brine, dried with Na₂SO₄, filtered and concentrated. The residue was purified using silica gel column chromatography eluting with EA in PE [gradient: 0-30%] to afford the title compound 5-bromo-2-methyl-2,3-dihydro-16-benzo[2,1-d][1,2]thiazole-1,1-dione (390 mg, 1.49 mmol, 52.6%) as a white solid. LCMS: ESI 262 [M+H]⁺.

Step D: Racemic cis 3-(5-((2-methyl-1,1-dioxido-2,3-dihydrobenzo[d]isothiazol-5-yl)amino)-1H-pyrazol-3-yl)cyclopentyl isopropylcarbamate To a solution of 5-bromo-2-methyl-2,3-dihydro-16-benzo[2,1-d][1,2]thiazole-1,1-dione (16.2 mg, 0.062 mmol) in dioxane (6 mL) was added racemic cis ethyl 5-amino-3-(3-((isopropylcarbamoyl)oxy)cyclopentyl)-1H-pyrazole-1-carboxylate (10 mg, 0.031 mmol), xantphos (3.59 mg, 0.006 mmol), Pd₂(dba)₃ (2.84 mg, 0.003 mmol) and Cs₂CO₃ (25.2 mg, 0.077 mmol). The reaction mixture was stirred at 100° C. under N₂ atmosphere for 1.5 h. The reaction was complete detected by LCMS. The cooled reaction mixture was filtered. The filtrate was concentrated. The residue was purified by prep-HPLC (C18, 10˜ 95% MeCN in H₂O with 0.1% HCOOH) to give racemic cis 3-(5-((2-methyl-1,1-dioxido-2,3-dihydrobenzo[d]isothiazol-5-yl)amino)-1H-pyrazol-3-yl)cyclopentyl isopropylcarbamate (2 mg, 0.005 mmol, 14.8%) as a white solid. LCMS: ESI m/z 434.2 [M+H]⁺. ¹H NMR (400 MHz, DMSO) δ 11.93 (s, 1H), 9.06 (s, 1H), 7.59 (d, J=8.4 Hz, 1H), 7.50 (s, 1H), 7.34 (d, J=8.4 Hz, 1H), 6.95 (d, J=7.2 Hz, 1H), 5.71 (s, 1H), 5.00 (s, 1H), 4.29 (s, 2H), 3.64-3.55 (m, 1H), 3.10-3.02 (m, 1H), 2.74 (s, 3H), 2.50-2.43 (m, 1H), 2.06-1.97 (m, 1H), 1.96-1.86 (m, 1H), 1.79-1.69 (m, 2H), 1.68-1.58 (m, 1H), 1.03 (d, J=6.4 Hz, 6H).

### Example 24: racemic cis 3-{5-[(1,1-dioxo-2,3-dihydro-1M6-benzothiophen-5-yl)amino]-4-methyl-1H-pyrazol-3-yl}cyclopentyl(prop-2-ylamino)methanoate

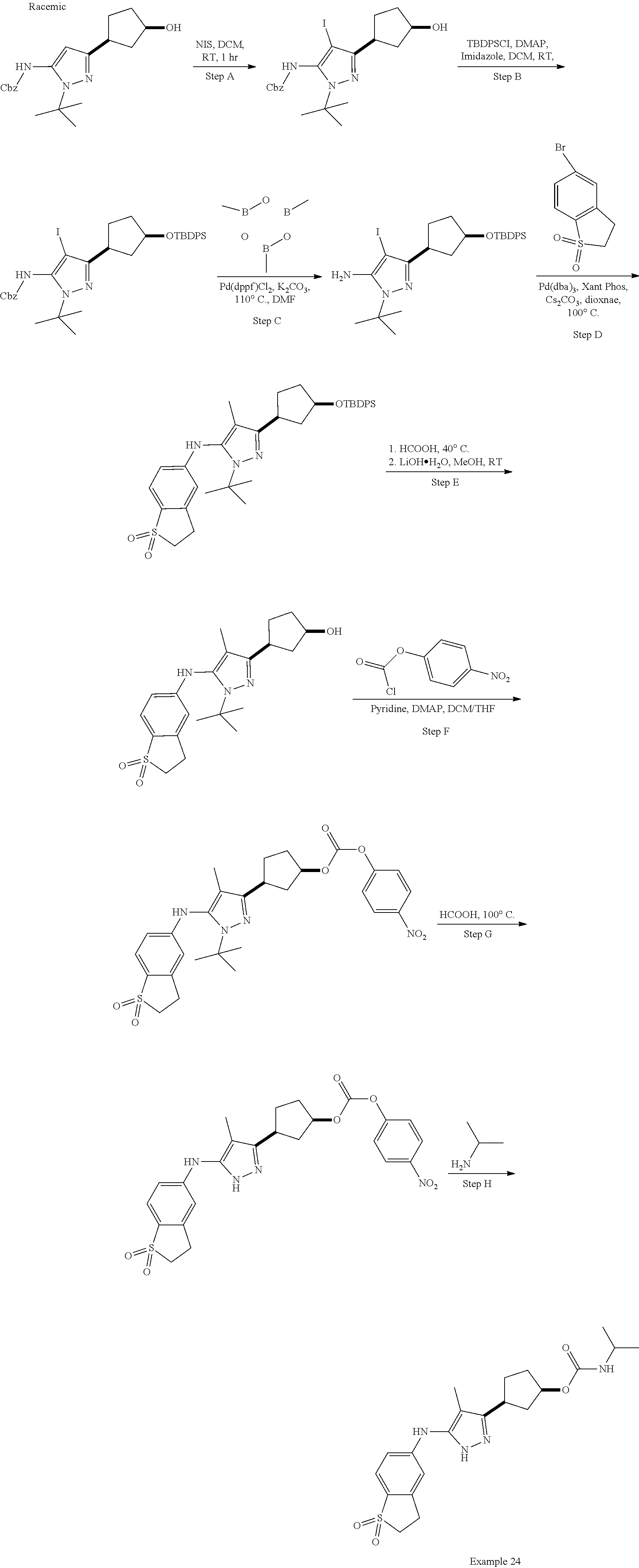

Step A: Racemic cis benzyl {[5-(3-hydroxycyclopentyl)-4-iodo-2-(2-methylprop-2-yl)pyrazol-3-yl]amino}methanoate To a solution of racemic cis benzyl (1-(tert-butyl)-3-(3-hydroxycyclopentyl)-1H-pyrazol-5-yl)carbamate (4 g, 11.2 mmol) in acetonitrile (50 mL) was added 1-iodotetrahydropyrrole-2,5-dione (2.52 g, 11.2 mmol), and the reaction was stirred at room temperature for 1 hr. The reaction was diluted with DCM (100 mL) and water (200 mL). The organic layer was separated, washed with brine, dry over Na₂SO₄ and concentrated. The residue was purified using silica gel column chromatography eluting with ethyl acetate in petroleum ether (1%-20%) to afford the title compound cis benzyl {[5-(3-hydroxycyclopentyl)-4-iodo-2-(2-methylprop-2-yl)pyrazol-3-yl]amino}methanoate (5.2 g, 10.7 mmol, 96.1%) as a yellow solid. LCMS:484.1 [M+H]⁺.

Step B: Racemic cis benzyl {[4-iodo-2-(2-methylprop-2-yl)-5-(3-{[(2-methylprop-2-yl)diphenylsilyl]oxy}cyclopentyl)pyrazol-3-yl]amino}methanoate To a solution of cis benzyl {[5-(3-hydroxycyclopentyl)-4-iodo-2-(2-methylprop-2-yl)pyrazol-3-yl]amino}methanoate (5.2 g, 10.7 mmol) in DCM (60 mL) were added DMAP (1.31 g, 10.7 mmol), 1H-imidazole (2.20 g, 32.2 mmol), and TBDPS chloride (3.34 g, 12.9 mmol) at 0° C. The reaction was stirred at room temperature for 18 hr. The reaction solution was dissolved in EtOAc (100 mL), washed with H₂O (200 mL) and brine, dried over sodium sulfate and concentrated. The residue was purified by silica gel chromatography (eluting with 60% ethyl acetate in petroleum ether) to afford benzyl {[4-iodo-2-(2-methylprop-2-yl)-5-(3-{[(2-methylprop-2-yl)diphenylsilyl]oxy}cyclopentyl)pyrazol-3-yl]amino}methanoate (5.0 g, 6.93 mmol, 64.4%) as a white oil. LCMS: ESI m/z 720.0 [M−H]⁻.

Step C: Racemic cis 4-methyl-2-(2-methylprop-2-yl)-5-(3-{[(2-methylprop-2-yl)diphenylsilyl]oxy}cyclopentyl)pyrazol-3-amine To a mixture of cis benzyl {[4-iodo-2-(2-methylprop-2-yl)-5-(3-{[(2-methylprop-2-yl)diphenylsilyl]oxy}cyclopentyl)pyrazol-3-yl]amino}methanoate (800 mg, 1.11 mmol) and 2,4,6-trimethyl-1,3,5,2,4,6-trioxatriborinane (2.53 mL, 8.86 mmol) in DMF (10 mL) were added K₂CO₃ (459 mg, 3.33 mmol) and Pd(dppf)Cl₂ (81.1 mg, 0.111 mmol). The reaction was stirred at 110° C. for 3 hr. The cooled reaction mixture was diluted with water and extracted with EA (50 mL*3). The combined organic layers were washed with brine, dried over Na₂SO₄ and concentrated. The residue was purified using silica gel column chromatography eluting with PE/EA (100/1˜12/1) to afford the title compound cis 4-methyl-2-(2-methylprop-2-yl)-5-(3-{[(2-methylprop-2-yl)diphenylsilyl]oxy}cyclopentyl)pyrazol-3-amine (140 mg, 0.294 mmol, 26.5%) as a pink oil. LCMS:476.3 [M+H]⁺

Step D: Racemic cis 5-((1-(tert-butyl)-3-(3-((tert-butyldiphenylsilyl)oxy)cyclopentyl)-4-methyl-1H-pyrazol-5-yl)amino)-2,3-dihydrobenzo[b]thiophene 1,1-dioxide To a stirred mixture of cis 4-methyl-2-(2-methylprop-2-yl)-5-(3-{[(2-methylprop-2-yl)diphenylsilyl]oxy}cyclopentyl)pyrazol-3-amine (140 mg, 0.294 mmol) in dioxane (10 mL) was added 5-bromo-2,3-dihydro-1λ6-benzothiophene-1,1-dione (109.08 mg, 0.441 mmol), xant-PHOS (34.05 mg, 0.059 mmol), Cs₂CO₃ (191 mg, 0.589 mmol) and Pd₂(dba)₃ (26.9 mg, 0.029 mmol). The reaction mixture was stirred at 120° C. under N₂ for 3 hr. The cooled reaction mixture was diluted with water and extracted with EA (20 mL*3). The combined organic layers were washed white brine and dried over Na₂SO₄, filtered, concentrated in vacuo. The residue was purified using silica gel column chromatography eluting with PE/EA (100/1˜ 3/1) to afford the title compound racemic cis 5-((1-(tert-butyl)-3-(3-((tert-butyldiphenylsilyl)oxy)cyclopentyl)-4-methyl-1H-pyrazol-5-yl)amino)-2,3-dihydrobenzo[b]thiophene 1,1-dioxide (110 mg, 0.171 mmol, 58.2%) as a yellowish green oil. LCMS: 642.5 [M+H]⁺

Step E: Racemic cis 5-{[5-(3-hydroxycyclopentyl)-4-methyl-2-(2-methylprop-2-yl)pyrazol-3-yl]amino}-2,3-dihydro-1λ\hat{ }6-benzothiophene-1,1-dione A solution of cis 5-((1-(tert-butyl)-3-(3-((tert-butyldiphenylsilyl)oxy)cyclopentyl)-4-methyl-1H-pyrazol-5-yl)amino)-2,3-dihydrobenzo[b]thiophene 1,1-dioxide (150 mg, 0.234 mmol) in HCOOH (5 mL) was stirred at 40° C. for 18 hr. The reaction mixture was concentrated. The residue was dissolved with methanol and adjusted to pH value 12-13 with aqueous 2 N lithium hydroxide aqueous solution. The reaction mixture was stirred at rt for 30 min and then diluted with EA and water. The organic layer was separated, washed with brine and concentrated. The residue was purified using silica gel column chromatography eluting with PE/EA (100/1˜ 1/100) to afford the title compound cis 5-{[5-(3-hydroxycyclopentyl)-4-methyl-2-(2-methylprop-2-yl)pyrazol-3-yl]amino}-2,3-dihydro-1λ6-benzothiophene-1,1-dione (90 mg, 0.223 mmol, 95.4%) as a yellow oil. LCMS: 404.3 [M+H]⁺

Step F: Racemic cis 3-{5-[(1,1-dioxo-2,3-dihydro-1λ\hat{ }6-benzothiophen-5-yl)amino]-4-methyl-1-(2-methylprop-2-yl)pyrazol-3-yl}cyclopentyl [(4-nitrophenyl)oxy]methanoate A suspension of cis 5-{[5-(3-hydroxycyclopentyl)-4-methyl-2-(2-methylprop-2-yl)pyrazol-3-yl]amino}-2,3-dihydro-16-benzothiophene-1,1-dione (90 mg, 0.223 mmol), pyridine (0.054 mL, 0.669 mmol), DMAP (2.72 mg, 0.022 mmol) and 4-nitrophenyl chloromethanoate (89.9 mg, 0.446 mmol) in THF (5 mL) and DCM (5 mL) was stirred at 50° C. overnight. The solvents were removed under vacuum. The residue was dissolved in ethyl acetate (50 mL), washed with brine (100 mL), dried over sodium sulfate and concentrated. The residue was purified by silica gel chromatography (eluting with 100% ethyl acetate in petroleum ether) to afford cis 3-{5-[(1,1-dioxo-2,3-dihydro-16-benzothiophen-5-yl)amino]-4-methyl-1-(2-methylprop-2-yl)pyrazol-3-yl}cyclopentyl [(4-nitrophenyl)oxy]methanoate (50 mg, 0.088 mmol, 39.4%) as a white solid. LCMS: 569.3 [M+H]⁺.

Step G: Racemic cis 3-{5-[(1,1-dioxo-2,3-dihydro-1λ\hat{ }6-benzothiophen-5-yl)amino]-4-methyl-1H-pyrazol-3-yl}cyclopentyl [(4-nitrophenyl)oxy]methanoate A solution of cis 3-{5-[(1,1-dioxo-2,3-dihydro-1λ6-benzothiophen-5-yl)amino]-4-methyl-1-(2-methylprop-2-yl)pyrazol-3-yl}cyclopentyl [(4-nitrophenyl)oxy]methanoate (50 mg, 0.088 mmol) in HCOOH (5 mL) was stirred at 100° C. for 18 hr. The cooled reaction mixture was concentrated in vacuo to afford the title compound cis 3-{5-[(1,1-dioxo-2,3-dihydro-16-benzothiophen-5-yl)amino]-4-methyl-1H-pyrazol-3-yl}cyclopentyl [(4-nitrophenyl)oxy]methanoate (50 mg, 0.073 mmol, 83.2%) as a yellow oil. LCMS: 513.2 [M+H]⁺

Step H: Racemic cis 3-{5-[(1,1-dioxo-2,3-dihydro-1λ\hat{ }6-benzothiophen-5-yl)amino]-4-methyl-1H-pyrazol-3-yl}cyclopentyl (prop-2-ylamino)methanoate A solution of cis 3-{5-[(1,1-dioxo-2,3-dihydro-16-benzothiophen-5-yl)amino]-4-methyl-1H-pyrazol-3-yl}cyclopentyl [(4-nitrophenyl)oxy]methanoate (50 mg, 0.073 mmol) in propan-2-amine (5 mL, 58.366 mmol) was stirred at room temperature for 2 hr. The reaction mixture was concentrated. The residue was purified by prep-HPLC (C18, 5%-95% MeCN in H₂O with 0.1% HCOOH) to give cis 3-{5-[(1,1-dioxo-2,3-dihydro-1λ6-benzothiophen-5-yl)amino]-4-methyl-1H-pyrazol-3-yl}cyclopentyl (prop-2-ylamino)methanoate (18.9 mg, 0.044 mmol, 59.7%) as a white solid. LCMS: 433 [M+H]⁺. ¹H NMR (400 MHz, DMSO) δ 11.82 (s, 1H), 8.34 (s, 1H), 7.45 (d, J=8.4 Hz, 1H), 7.29-7.19 (m, 2H), 6.97 (d, J=8.4 Hz, 1H), 4.99 (s, 1H), 3.65-3.55 (m, 1H), 3.46 (t, J=6.8 Hz, 2H), 3.21 (t, J=6.8 Hz, 2H), 3.15-3.02 (m, 1H), 2.44-2.35 (m, 1H), 2.03-1.91 (m, 2H), 1.88 (s, 3H), 1.83-1.63 (m, 3H), 1.04 (d, J=6.4 Hz, 6H).

### Example 25: (1R,3S)-3-{5-[(1-oxo-2,3-dihydro-1H-inden-5-yl)amino]-2H-pyrazol-3-yl}cyclopentyl (prop-2-ylamino)methanoate

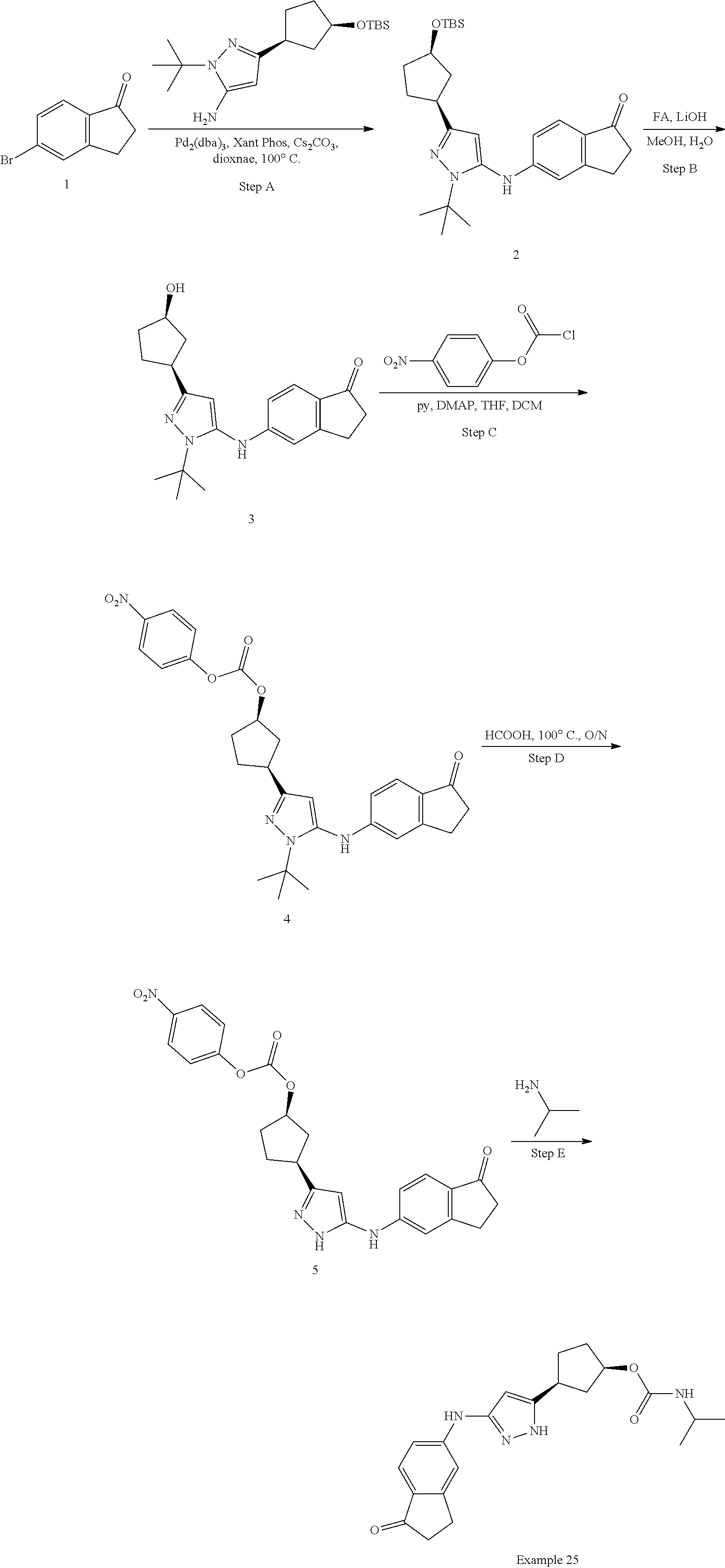

Step A: 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-inden-1-one To a solution of 5-bromo-2,3-dihydro-1H-inden-1-one (0.169 mL, 0.948 mmol) in dioxane (10 mL) were added 5-[(1S,3R)-3-{[dimethyl(2-methylprop-2-yl)silyl]oxy}cyclopentyl]-2-(2-methylprop-2-yl)pyrazol-3-amine (319 mg, 0.948 mmol), Cs₂CO₃ (617 mg, 1.89 mmol), Xant-PHOS (109 mg, 0.190 mmol) and Pd₂(dba)₃ (86.7 mg, 0.095 mmol). The reaction mixture was stirred at 120° C. under N₂ for 3 hr. The cooled reaction mixture was diluted with EA and water. The organic layer was separated, washed with further brine, and concentrated. The residue was purified using silica gel column chromatography eluting with ethyl acetate in petroleum ether to afford the title compound 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-inden-1-one (200 mg, 0.428 mmol, 45.1%) as a yellow oil. LCMS: ESI m/z 468 [M+H]⁺.

Step B: 5-({5-[(1S,3R)-3-hydroxycyclopentyl]-2-(2-methylprop-2-yl)pyrazol-3-yl}amino)-2,3-dihydro-1H-inden-1-one A mixture of 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-inden-1-one (150 mg, 0.321 mmol) in formic acid (6 mL) was stirred at RT for 1 h, then concentrated under reduced pressure. The residue was treated with a mixture of lithium hydroxide hydrate (26.9 mg, 0.642 mmol) in MeOH (9 mL)/H₂O (3 mL). The resulting mixture was stirred at rt for 2 h, the mixture was concentrated under reduced pressure. The residue was poured into water and extracted with EtOAc. The combined organic phase was washed with brine, dried with Na₂SO₄, filtered and concentrated. The residue was purified by chromatography to give 5-({5-[(1S,3R)-3-hydroxycyclopentyl]-2-(2-methylprop-2-yl)pyrazol-3-yl}amino)-2,3-dihydro-1H-inden-1-one (80 mg, 0.226 mmol, 70.5%) as a colorless oil. LCMS: ESI m/z 354 [M+H]⁺.

Step C: (1R,3S)-3-[1-(2-methylprop-2-yl)-5-[(1-oxo-2,3-dihydro-1H-inden-5-yl)amino]pyrazol-3-yl]cyclopentyl [(4-nitrophenyl)oxy]methanoate To a solution of 5-({5-[(1S,3R)-3-hydroxycyclopentyl]-2-(2-methylprop-2-yl)pyrazol-3-yl}amino)-2,3-dihydro-1H-inden-1-one (100 mg, 0.283 mmol) in DCM (3 mL) and THF (3 mL) were added 4-nitrophenyl chloromethanoate (114 mg, 0.566 mmol), Py (0.046 mL, 0.566 mmol) and DMAP (3.46 mg, 0.028 mmol). The reaction mixture was stirred under at 25° C. for 2 hr. The reaction solution was diluted with EA, washed with water and brine, dried over anhydrous Na₂SO₄ and concentrated. The residue was purified by flash chromatography to afford (1R,3S)-3-[1-(2-methylprop-2-yl)-5-[(1-oxo-2,3-dihydro-1H-inden-5-yl)amino]pyrazol-3-yl]cyclopentyl [(4-nitrophenyl)oxy]methanoate (100 mg, 0.193 mmol, 68.1%) as a yellow solid. LCMS: ESI m/z 519 [M+H]⁺.

Step D: (1R,3S)-3-{5-[(1-oxo-2,3-dihydro-1H-inden-5-yl)amino]-1H-pyrazol-3-yl}cyclopentyl [(4-nitrophenyl)oxy]methanoate A solution of (1R,3S)-3-[1-(2-methylprop-2-yl)-5-[(1-oxo-2,3-dihydro-1H-inden-5-yl)amino]pyrazol-3-yl]cyclopentyl [(4-nitrophenyl)oxy]methanoate (100 mg, 0.193 mmol) in formic acid (5 mL) was stirred at 100° C. overnight. The cooled reaction mixture was concentrated to afford (1R,3S)-3-{5-[(1-oxo-2,3-dihydro-1H-inden-5-yl)amino]-1H-pyrazol-3-yl}cyclopentyl [(4-nitrophenyl)oxy]methanoate (70 mg, 0.151 mmol, 78.5%) as a yellow oil. LCMS: ESI m/z 463 [M+H]⁺.

Step E: (1R,3S)-3-{5-[(1-oxo-2,3-dihydro-1H-inden-5-yl)amino]-2H-pyrazol-3-yl}cyclopentyl (prop-2-ylamino)methanoate To a solution of (1R,3S)-3-{5-[(1-oxo-2,3-dihydro-1H-inden-5-yl)amino]-1H-pyrazol-3-yl}cyclopentyl [(4-nitrophenyl)oxy]methanoate (70 mg, 0.151 mmol) in THF (5 mL) was added propan-2-amine (0.026 mL, 0.303 mmol). The reaction mixture was stirred at rt for 1 h. The reaction concentrated in vacuo. The residue was purified by prep-HPLC to afford the title compound (1R,3S)-3-{5-[(1-oxo-2,3-dihydro-1H-inden-5-yl)amino]-2H-pyrazol-3-yl}cyclopentyl (prop-2-ylamino)methanoate (53 mg, 0.139 mmol, 91.5%) as a white solid. LCMS: ESI m/z 383 [M+H]⁺. ¹H NMR (400 MHz, DMSO) δ 11.94 (s, 1H), 9.07 (s, 1H), 7.53 (s, 1H), 7.43 (d, J=8.4 Hz, 1H), 7.17 (d, J=8.4 Hz, 1H), 6.95 (d, J=7.2 Hz, 1H), 5.73 (d, J=1.6 Hz, 1H), 5.00 (s, 1H), 3.63-3.53 (m, 1H), 3.13-3.01 (m, 1H), 3.00-2.93 (m, 2H), 2.48-2.42 (m, 1H), 2.008-1.97 (m, 1H), 1.96-1.86 (m, 1H), 1.81-1.68 (m, 2H), 1.68-1.58 (m, 1H), 1.03 (d, J=6.4 Hz, 6H).

### Example 26: (1R,3S)-3-(3-((1,1-dioxidothiochroman-6-yl)amino)-1H-pyrazol-5-yl)cyclopentyl isopropylcarbamate

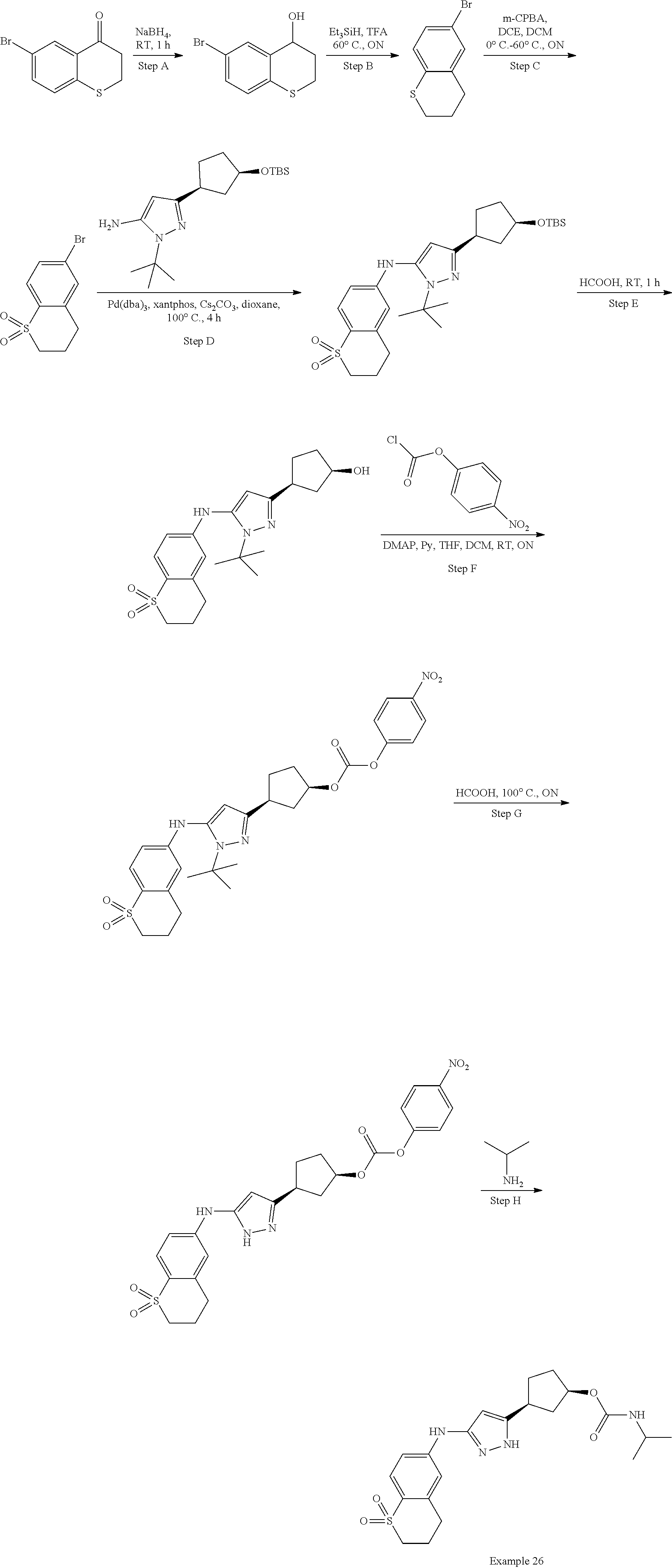

Step A: 6-bromo-3,4-dihydro-2H-1-benzothiin-4-ol To a solution of 6-bromo-3,4-dihydro-2H-1-benzothiin-4-one (1 g, 4.11 mmol) in MeOH (5 mL) was added NaBH₄ (0.28 g, 8.23 mmol). The reaction mixture was stirred at room temperature for 1 hr. The reaction was diluted with EA and water. The organic layer was separated, washed with brine and concentrated in vacuo to afford the title compound 6-bromo-3,4-dihydro-2H-1-benzothiin-4-ol (900 mg, 3.67 mmol, 89.3%) as a brown oil. LCMS: m/z 245.13 [M+H]⁺.

Step B: 6-bromo-3,4-dihydro-2H-1-benzothiine To a solution of 6-bromo-3,4-dihydro-2H-1-benzothiin-4-ol (900 mg, 3.67 mmol) in TFA (10 mL) was added Et₃SiH (2.97 mL, 18.4 mmol). The reaction mixture was stirred at 60° C. overnight. The cooled reaction mixture was diluted with EA and water. The organic layer was separated, washed with brine and concentrated. The residue was purified using silica gel column chromatography eluted with ethyl acetate in petroleum ether (gradient: 0-1%) to afford the title compound 6-bromo-3,4-dihydro-2H-1-benzothiine (840 mg, 3.67 mmol, 99.9%) as a colorless oil. LCMS: m/z 229.13 [M+H]⁺.

Step C: 6-bromothiochromane 1,1-dioxide To a solution of 6-bromo-3,4-dihydro-2H-1-benzothiine (900 mg, 3.93 mmol) in 1,2-dichloroethane (10 mL) was added 3-Chloroperoxybenzoic acid (2.39 g, 11.8 mmol). The reaction was stirred at 60° C. overnight. The reaction was diluted with EA and water. The organic layer was separated, washed with saturated NaHCO₃ solution and concentrated in vacuo. The residue was purified using silica gel column chromatography eluted with ethyl acetate in petroleum ether (gradient:0-30%) to afford the title compound 6-bromothiochromane 1,1-dioxide (780 mg, 2.99 mmol, 76.1%) as a pink solid. LCMS: m/z 261.13 [M+H]⁺.

Step D: 6-((1-(tert-butyl)-3-((1S,3R)-3-((tert-butyldimethylsilyl)oxy)cyclopentyl)-1H-pyrazol-5-yl)amino)thiochromane 1,1-dioxide To a stirred solution of 6-bromothiochromane 1,1-dioxide (300 mg, 1.15 mmol) in dioxane (10 mL) were added 5-[(1S,3R)-3-{[dimethyl(2-methylprop-2-yl)silyl]oxy}cyclopentyl]-2-(2-methylprop-2-yl)pyrazol-3-amine (388 mg, 1.15 mmol), Pd₂(dba)₃ (105 mg, 0.115 mmol), Xant-PHOS (133 mg, 0.230 mmol) and Cs₂CO₃ (1.12 g, 3.48 mmol). The reaction was stirred at 100° C. under N₂ for 4 hr. The cooled reaction mixture was diluted with EA and water. The organic layer was separated, washed with saturated brine and concentrated in vacuo. The residue was purified using silica gel column chromatography eluted with ethyl acetate in petroleum ether (gradient: 0-30%) to afford the title compound 6-((1-(tert-butyl)-3-((1S,3R)-3-((tert-butyldimethylsilyl)oxy)cyclopentyl)-1H-pyrazol-5-yl)amino)thiochromane 1,1-dioxide (400 mg, 0.772 mmol, 67.2%) as a yellow solid. LCMS: m/z 517.80 [M+H]⁺.

Step E: 6-((1-(tert-butyl)-3-((1S,3R)-3-hydroxycyclopentyl)-1H-pyrazol-5-yl)amino)thiochromane 1,1-dioxide A solution of 6-((1-(tert-butyl)-3-((1S,3R)-3-((tert-butyldimethylsilyl)oxy)cyclopentyl)-1H-pyrazol-5-yl)amino)thiochromane 1,1-dioxide (150 mg, 0.240 mmol) in formic acid (5 mL) was stirred at room temperature for 1 h and then concentrated in vacuo. The residue was dissolve with methanol and adjusted to pH value 12-13 with aq. 2 N lithium hydroxide aqueous solution. The reaction was stirred at rt for 30 min and then diluted with EA and water. The organic layer was separated, washed with brine and concentrated. The residue was purified using silica gel column chromatography eluted with ethyl acetate in petroleum ether (gradient: 0-50%) to afford the title compound 6-((1-(tert-butyl)-3-((1S,3R)-3-hydroxycyclopentyl)-1H-pyrazol-5-yl)amino)thiochromane 1,1-dioxide (90 mg, 0.176 mmol, 73.4%) as a yellow solid. LCMS: m/z 510.65 [M+H]⁺.

Step F: (1R,3S)-3-(1-(tert-butyl)-5-((1,1-dioxidothiochroman-6-yl)amino)-1H-pyrazol-3-yl)cyclopentyl (4-nitrophenyl) carbonate To a stirred solution of 6-((1-(tert-butyl)-3-((1S,3R)-3-hydroxycyclopentyl)-1H-pyrazol-5-yl)amino)thiochromane 1,1-dioxide (300 mg, 0.743 mmol) in THF (5 mL) and DCM (5 mL) were added 4-nitrophenyl chloromethanoate (225 mg, 1.12 mmol), DMAP (9.08 mg, 0.074 mmol) and Py (118 mg, 1.49 mmol). The reaction mixture was stirred at room temperature overnight. The reaction mixture was diluted with DCM and water. The organic layer was separated, washed with brine and concentrated. The residue was purified using silica gel column chromatography eluted with ethyl acetate in petroleum ether (gradient: 0-30%) to afford the title compound (1R,3S)-3-(1-(tert-butyl)-5-((1,1-dioxidothiochroman-6-yl)amino)-1H-pyrazol-3-yl)cyclopentyl (4-nitrophenyl) carbonate (330 mg, 0.580 mmol, 78.1%) as a yellow solid. LCMS: m/z 568.64 [M+H]⁺.

Step G: (1R,3S)-3-(5-((1,1-dioxidothiochroman-6-yl)amino)-1H-pyrazol-3-yl)cyclopentyl (4-nitrophenyl) carbonate A solution of (1R,3S)-3-(1-(tert-butyl)-5-((1,1-dioxidothiochroman-6-yl)amino)-1H-pyrazol-3-yl)cyclopentyl (4-nitrophenyl) carbonate (150 mg, 0.264 mmol) in formic acid (5 mL) was stirred at 100° C. overnight. The reaction mixture was concentrated to afford the title compound (1R,3S)-3-(5-((1,1-dioxidothiochroman-6-yl)amino)-1H-pyrazol-3-yl)cyclopentyl (4-nitrophenyl) carbonate (130 mg, 0.254 mmol, 96.2%) as a brown oil. LCMS: m/z 512.54 [M+H]⁺.

Step H: (1R,3S)-3-(3-((1,1-dioxidothiochroman-6-yl)amino)-1H-pyrazol-5-yl)cyclopentyl isopropylcarbamate A solution of (1R,3S)-3-(5-((1,1-dioxidothiochroman-6-yl)amino)-1H-pyrazol-3-yl)cyclopentyl (4-nitrophenyl) carbonate (150 mg, 0.293 mmol) in propan-2-amine (17.3 mg, 0.293 mmol) was stirred at room temperature for 1 h. The reaction was concentrated in vacuo. The residue was purified by prep-HPLC (C18, 0-70% acetonitrile in H₂O) to afford the title compound (1R,3S)-3-(3-((1,1-dioxidothiochroman-6-yl)amino)-1H-pyrazol-5-yl)cyclopentyl isopropylcarbamate (31.1 mg, 0.072 mmol, 24.5%) as a white solid. LCMS: m/z 432.54 [M+H]⁺. ¹H NMR (400 MHz, DMSO) δ 11.88 (s, 1H), 8.85 (s, 1H), 7.54 (d, J=8.8 Hz, 1H), 7.27 (d, J=8.0 Hz, 2H), 6.91 (dd, J=26.8, 8.0 Hz, 1H), 5.67 (s, 1H), 4.99 (s, 1H), 3.62-3.52 (m, 1H), 3.42-3.34 (m, 2H), 3.12-2.98 (m, 1H), 2.89 (t, J=6.4 Hz, 2H), 2.48-2.40 (m, 1H), 2.27 (dt, J=12.0, 5.6 Hz, 2H), 2.02-1.82 (m, 2H), 1.75-1.54 (m, 3H), 1.03 (d, J=6.4 Hz, 6H).

### Example 27: (1s,4s)-4-{5-[(1,1-dioxo-2,3-dihydro-1λ<sup>6</sup>-benzo[b]thiophen-6-yl)amino]-2H-pyrazol-3-yl}cyclohexyl (prop-2-ylamino)methanoate

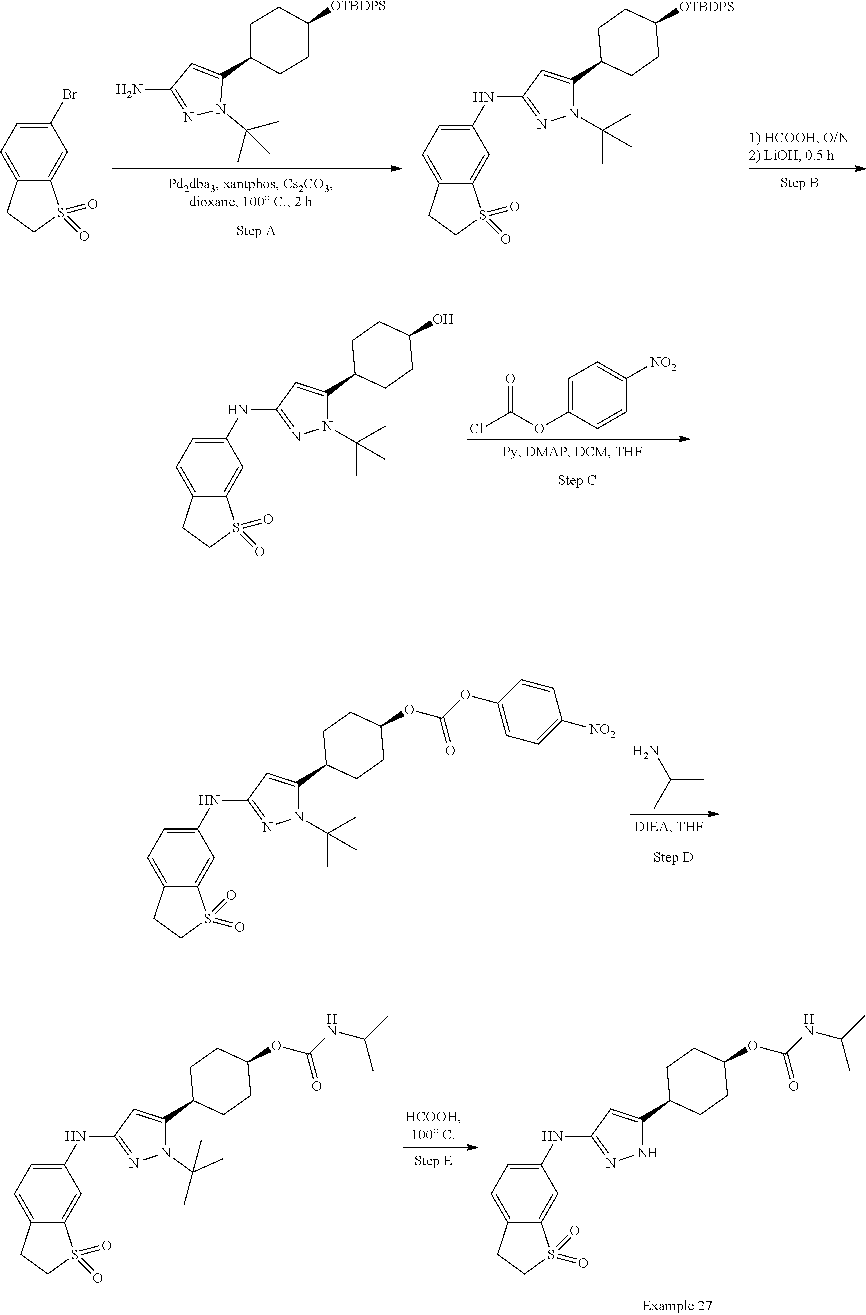

Step A. 6-({5-[(1s,4s)-4-{[(2-methylprop-2-yl)diphenylsilyl]oxy}cyclohexyl]-1-(2-methylprop-2-yl)pyrazol-3-yl}amino)-2,3-dihydro-1,<sup>6</sup>-benzo[b]thiophene-1,1-dione. To a stirred solution of 6-bromo-2,3-dihydro-1⁶-benzo[2,1-b]thiophene-1,1-dione (104 mg, 0.42 mmol) in dioxane (2 mL) was added 5-[(1s,4s)-4-{[(2-methylprop-2-yl)diphenylsilyl]oxy}cyclohexyl]-1-(2-methylprop-2-yl)pyrazol-3-amine (200 mg, 0.42 mmol), Pd₂(dba)₃ (38 mg, 0.04 mmol), Xant-PHOS (49 mg, 0.08 mmol) and Cs₂CO₃ (342 mg, 1.1 mmol) at rt. The reaction mixture was stirred at 100° C. under N₂ for 2 h. The cooled reaction mixture was poured into water (30 mL) and extracted with EtOAc (20 mL*2). The combined organic phase was washed with brine, dried with Na₂SO₄, filtered and concentrated. The residue was purified by chromatography (silica gel, 0-60%, EtOAc in PE) to give 6-({5-[(1s,4s)-4-{[(2-methylprop-2-yl)diphenylsilyl]oxy}cyclohexyl]-1-(2-methylprop-2-yl)pyrazol-3-yl}amino)-2,3-dihydro-1λ⁶-benzothiophene-1,1-dione (250 mg, 0.39 mmol, 92%) as a yellow solid. LCMS: m/z 642 [M+H]⁺.

Step B. 6-({5-[(1s,4s)-4-hydroxycyclohexyl]-1-(2-methylprop-2-yl)pyrazol-3-yl}amino)-2,3-dihydro-1λ<sup>6</sup>-benzo[b]thiophene-1,1-dione. A mixture of 6-({5-[(1s,4s)-4-{[(2-methylprop-2-yl)diphenylsilyl]oxy}cyclohexyl]-1-(2-methylprop-2-yl)pyrazol-3-yl}amino)-2,3-dihydro-1⁶-benzothiophene-1,1-dione (260 mg, 0.41 mmol) in HCOOH (5 mL) was stirred at rt overnight. The reaction mixture was concentrated. Then EtOH (1 mL)/H₂O (3 mL) and LiOH (15 mg, 3.2 mmol) was added. The following mixture was stirred at rt for 2 h and then diluted with water (15 mL), extracted with EtOAc (10 mL*2). The combined organic phase was washed with brine, dried with Na₂SO₄, filtered and concentrated. The residue was purified by chromatography (silica gel, 0-10%, MeOH in DCM) to give 6-({5-[(1s,4s)-4-hydroxycyclohexyl]-1-(2-methylprop-2-yl)pyrazol-3-yl}amino)-2,3-dihydro-1λ⁶-benzothiophene-1,1-dione (130 mg, 0.32 mmol, 79%) as a yellow solid. LCMS: m/z 404 [M+H]⁺.

Step C. (1s,4s)-4-{5-[(1,1-dioxo-2,3-dihydro-1λ<sup>6</sup>-benzo[b]thiophen-6-yl)amino]-2-(2-methylprop-2-yl)pyrazol-3-yl}cyclohexyl [(4-nitrophenyl)oxy]methanoate. To a stirred solution of 6-({5-[(1s,4s)-4-hydroxycyclohexyl]-1-(2-methylprop-2-yl)pyrazol-3-yl}amino)-2,3-dihydro-1λ⁶-benzothiophene-1,1-dione (130 mg, 0.32 mmol) in DCM (3 mL)/THF (3 mL) was added 4-nitrophenyl chloromethanoate (324 mg, 1.6 mmol), DMAP (13 mg, 0.11 mmol) and pyridine (2 mL, 24 mmol) slowly at rt. After stirred at rt overnight, the cooled mixture was poured into water (30 mL) and extracted with EtOAc (20 mL*2). The combined organic phase was washed with brine, dried with Na₂SO₄, filtered and concentrated. The residue was purified by chromatography (silica gel, 0-10%, MeOH in DCM) to give (1s,4s)-4-{5-[(1,1-dioxo-2,3-dihydro-1λ⁶-benzothiophen-6-yl)amino]-2-(2-methylprop-2-yl)pyrazol-3-yl}cyclohexyl [(4-nitrophenyl)oxy]methanoate (170 mg, 0.3 mmol, 92%) as a yellow solid. LCMS: m/z 569 [M+H]⁺.

Step D. (1s,4s)-4-{5-[(1,1-dioxo-2,3-dihydro-1λ<sup>6</sup>-benzo[b]thiophen-6-yl)amino]-2-(2-methylprop-2-yl)pyrazol-3-yl}cyclohexyl (prop-2-ylamino)methanoate. To a stirred solution of (1s,4s)-4-{5-[(1,1-dioxo-2,3-dihydro-1λ⁶-benzothiophen-6-yl)amino]-2-(2-methylprop-2-yl)pyrazol-3-yl}cyclohexyl [(4-nitrophenyl)oxy]methanoate (170 mg, 0.3 mmol) in THF (10 mL) was added DIPEA (0.5 mL, 0.2 mmol) and propan-2-amine (0.5 mL, 3 mmol) slowly at rt. After stirred at rt for 1 h, the cooled mixture was poured into water (30 mL) and extracted with EtOAc (20 mL*2). The combined organic phase was washed with brine, dried with Na₂SO₄, filtered and concentrated. The residue was purified by chromatography (silica gel, 0-10%, MeOH in DCM) to give (1s,4s)-4-{5-[(1,1-dioxo-2,3-dihydro-1λ⁶-benzothiophen-6-yl)amino]-2-(2-methylprop-2-yl)pyrazol-3-yl}cyclohexyl (prop-2-ylamino)methanoate (130 mg, 0.27 mmol, 89%) as a yellow oil. LCMS: m/z 489 [M+H]⁺.

Step E. (1s,4s)-4-{5-[(1,1-dioxo-2,3-dihydro-1λ<sup>6</sup>-benzo[b]thiophen-6-yl)amino]-2H-pyrazol-3-yl}cyclohexyl (prop-2-ylamino)methanoate. A mixture of (1s,4s)-4-{5-[(1,1-dioxo-2,3-dihydro-1λ⁶-benzothiophen-6-yl)amino]-2-(2-methylprop-2-yl)pyrazol-3-yl}cyclohexyl (prop-2-ylamino)methanoate (120 mg, 0.25 mmol) in HCOOH (3 mL) was stirred at 100° C. for 5 h. The reaction mixture was concentrated. The residue was purified by prep-HPLC (C18, 40˜ 90% MeCN in H₂O with 0.1% TFA) and then by prep-TLC (DCM: MeOH=10:1) to give (1s,4s)-4-{5-[(1,1-dioxo-2,3-dihydro-1⁶-benzothiophen-6-yl)amino]-2H-pyrazol-3-yl}cyclohexyl (prop-2-ylamino)methanoate (18 mg, 0.042 mmol, 17%) as a white solid. LCMS: m/z 433.2 [M+H]⁺. ¹H NMR (400 MHz, DMSO) δ 11.89 (s, 1H), 8.88 (s, 1H), 8.02 (s, 1H), 7.43 (dd, J=8.4, 1.6 Hz, 1H), 7.35 (d, J=8.4 Hz, 1H), 6.96 (d, J=7.2 Hz, 1H), 5.65 (s, 1H), 4.81 (s, 1H), 3.71-3.61 (m, 1H), 3.58 (dd, J=8.8, 4.8 Hz, 2H), 3.26 (t, J=6.8 Hz, 2H), 2.74 (s, 1H), 1.96-1.80 (m, 4H), 1.80-1.60 (m, 4H), 1.11 (d, J=6.4 Hz, 6H).

### Example 28: (1s,3s)-3-(3-((2,2-dioxido-1,3-dihydrobenzo[c]thiophen-5-yl)amino)-1H-pyrazol-5-yl)cyclobutyl isopropylcarbamate

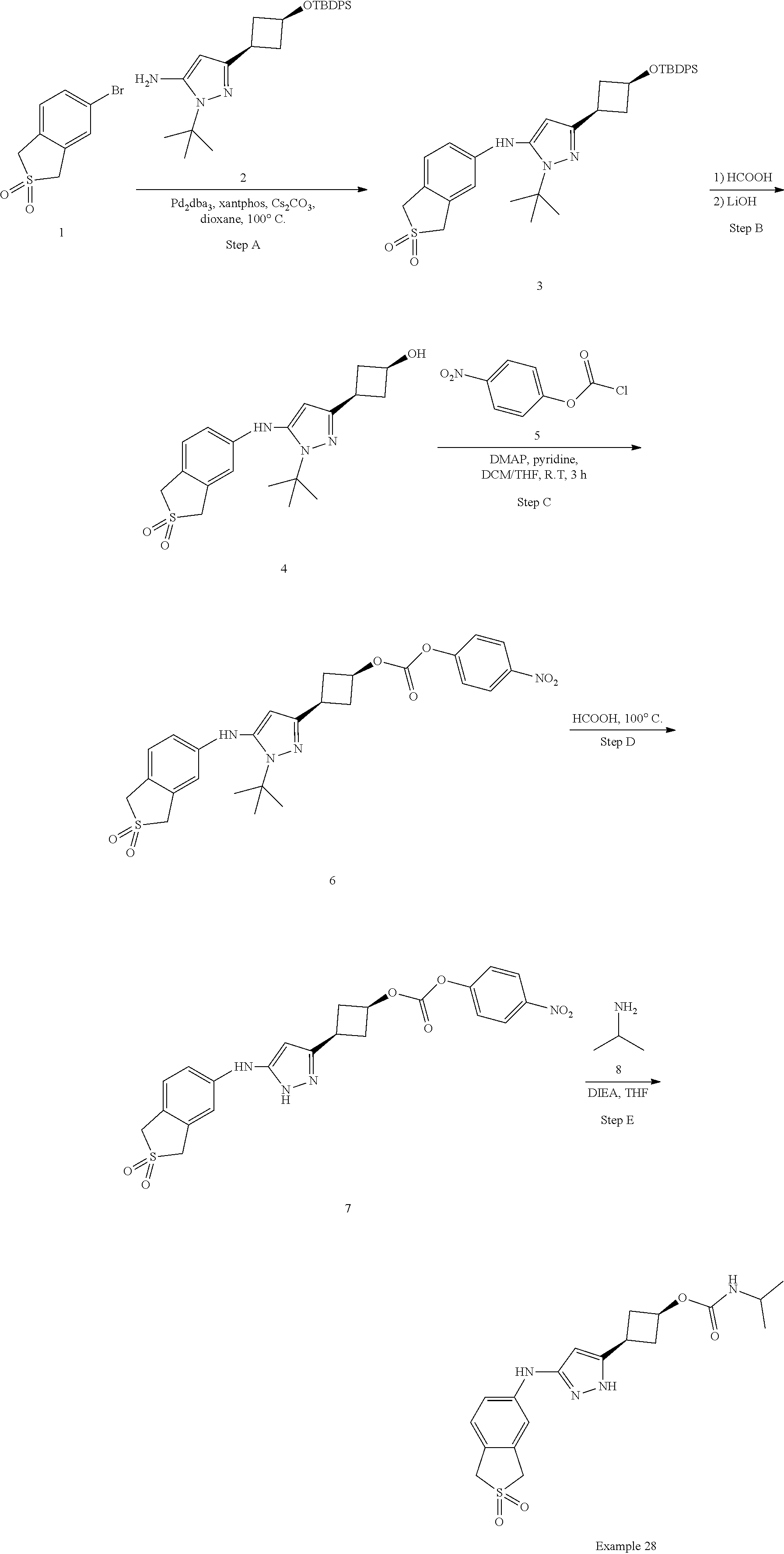

Step A: 5-((1-(tert-butyl)-3-((1s,3s)-3-((tert-butyldiphenylsilyl)oxy)cyclobutyl)-1H-pyrazol-5-yl)amino)-1,3-dihydrobenzo[c]thiophene 2,2-dioxide To a solution of 5-bromo-1,3-dihydrobenzo[c]thiophene 2,2-dioxide (450 mg, 1.82 mmol) in dioxane (15 mL) was added 1-(tert-butyl)-3-((1s,3s)-3-((tert-butyldiphenylsilyl)oxy)cyclobutyl)-1H-pyrazol-5-amine (200 mg, 0.447 mmol), Cs₂CO₃ (437 mg, 1.34 mmol), Pd₂(dba)₃ (40.9 mg, 0.0450 mmol) and Xantphos (51.7 mg, 0.089 mmol). The reaction mixture was stirred at 100° C. under N₂ atmosphere for 1 hr. The reaction was complete detected by LCMS. The cooled reaction mixture was dissolved in EA (10 mL*3), washed with H₂O (20 mL) and brine, dried over sodium sulfate, filtered and concentrated. The residue was purified by silica gel chromatography (eluting with 0-50% EA in PE) to afford 5-((1-(tert-butyl)-3-((1s,3s)-3-((tert-butyldiphenylsilyl)oxy)cyclobutyl)-1H-pyrazol-5-yl)amino)-1,3-dihydrobenzo[c]thiophene 2,2-dioxide (250 mg, 0.407 mmol, 91.2%) as a yellow solid. LCMS: ESI m/z 614.89 [M+H]⁺.

Step B: 5-((1-(tert-butyl)-3-((1s,3s)-3-hydroxycyclobutyl)-1H-pyrazol-5-yl)amino)-1,3-dihydrobenzo[c]thiophene 2,2-dioxide A solution of 5-((1-(tert-butyl)-3-((1s,3s)-3-((tert-butyldiphenylsilyl)oxy)cyclobutyl)-1H-pyrazol-5-yl)amino)-1,3-dihydrobenzo[c]thiophene 2,2-dioxide (245 mg, 0.399 mmol) in formic acid (4 mL) was stirred at room temperature overnight. The reaction was complete detected by LCMS. The solvents were removed under vacuum. Then add EtOH (2.5 mL), H₂O (2.5 mL) and Lithium Hydroxide Monohydrate (50.2 mg, 1.20 mmol) to alkaline pH. The mixture was stirred at room temperature for 1 hr. LCMS showed the reaction was completed. The residue was dissolved in EA (5 mL*3), washed with H₂O (10 mL) and brine, dried over sodium sulfate, filtered and concentrated. The residue was purified by silica gel chromatography (eluting with 0-12% MeOH in DCM) to afford 5-((1-(tert-butyl)-3-((1s,3s)-3-hydroxycyclobutyl)-1H-pyrazol-5-yl)amino)-1,3-dihydrobenzo[c]thiophene 2,2-dioxide (130 mg, 0.346 mmol, 86.8%) as a white solid. LCMS: ESI m/z 376.49 [M+H]⁺.

Step C: (1s,3s)-3-(1-(tert-butyl)-5-((2,2-dioxido-1,3-dihydrobenzo[c]thiophen-5-yl)amino)-1H-pyrazol-3-yl)cyclobutyl (4-nitrophenyl) carbonate A suspension of 5-((1-(tert-butyl)-3-((1s,3s)-3-hydroxycyclobutyl)-1H-pyrazol-5-yl)amino)-1,3-dihydrobenzo[c]thiophene 2,2-dioxide (130 mg, 0.346 mmol), DMAP (4.23 mg, 0.0350 mmol), Py (0.0840 mL, 1.04 mmol) and 4-nitrophenyl chloromethanoate (349 mg, 1.73 mmol) in DCM (3 mL) and THF (3 mL) was stirred at room temperature for 3 hrs. The reaction was completed by LCMS. The solvents were removed under vacuum. The residue was dissolved in EA (5 mL*3), washed with H₂O (5 mL) and brine, dried over sodium sulfate, filtered, concentrated and purified by silica gel chromatography (eluting with 0-12% MeOH in DCM) to afford (1s,3s)-3-(1-(tert-butyl)-5-((2,2-dioxido-1,3-dihydrobenzo[c]thiophen-5-yl)amino)-1H-pyrazol-3-yl)cyclobutyl (4-nitrophenyl) carbonate (170 mg, 0.314 mmol, 90.8%) as a white solid. LCMS: ESI m/z 541.59 [M+H]⁺.

Step D: (1s,3s)-3-(5-((2,2-dioxido-1,3-dihydrobenzo[c]thiophen-5-yl)amino)-1H-pyrazol-3-yl)cyclobutyl (4-nitrophenyl) carbonate A solution of (1s,3s)-3-(1-(tert-butyl)-5-((2,2-dioxido-1,3-dihydrobenzo[c]thiophen-5-yl)amino)-1H-pyrazol-3-yl)cyclobutyl (4-nitrophenyl) carbonate (170 mg, 0.314 mmol) in formic acid (4 mL) was stirred at 100° C. overnight. LCMS showed the reaction was completed. The cooled reaction mixture was concentrated to give crude (1s,3s)-3-(5-((2,2-dioxido-1,3-dihydrobenzo[c]thiophen-5-yl)amino)-1H-pyrazol-3-yl)cyclobutyl (4-nitrophenyl) carbonate (150 mg, 0.310 mmol, 98.4%) as a yellow oil. LCMS: ESI m/z 485.48 [M+H]⁺.

Step E: (1s,3s)-3-(3-((2,2-dioxido-1,3-dihydrobenzo[c]thiophen-5-yl)amino)-1H-pyrazol-5-yl)cyclobutyl isopropylcarbamate To a suspension of (1s,3s)-3-(5-((2,2-dioxido-1,3-dihydrobenzo[c]thiophen-5-yl)amino)-1H-pyrazol-3-yl)cyclobutyl (4-nitrophenyl) carbonate (150 mg, 0.310 mmol) in THF (4 mL) was added propan-2-amine (0.133 mL, 1.55 mmol) and DIEA (0.154 mL, 0.929 mmol). The reaction mixture was stirred at room temperature for 3 hrs. LCMS showed the reaction had been completed. The residue was dissolved in EA (5 mL*3), washed with H₂O (10 mL) and brine, dried over sodium sulfate, filtered, concentrated and purified by silica gel chromatography (eluting with 0-12% MeOH in DCM) and prep-HPLC (C18, 15˜ 95% MeCN in H₂O with 0.1% FA) to afford (1s,3s)-3-(3-((2,2-dioxido-1,3-dihydrobenzo[c]thiophen-5-yl)amino)-1H-pyrazol-5-yl)cyclobutyl isopropylcarbamate (17.8 mg, 0.0440 mmol, 14.2%) as a white solid. LCMS: ESI m/z 405.49 [M+H]⁺. ¹H NMR (400 MHz, DMSO) δ 8.55 (s, 1H), 7.36 (s, 1H), 7.21-7.13 (m, 2H), 7.06 (t, J=9.6 Hz, 1H), 5.71 (s, 1H), 4.79 (dd, J=14.8, 7.6 Hz, 1H), 4.41 (s, 2H), 4.33 (s, 2H), 3.54-3.52 (m, 1H), 3.12-3.00 (m, 1H), 2.69-2.55 (m, 2H), 2.08 (dd, J=17.6, 9.2 Hz, 2H), 1.04 (d, J=6.4 Hz, 6H).

### Example 29: (1R,3S)-3-{5-[(1-cyano-2,3-dihydro-1H-inden-5-yl)amino]-2H-pyrazol-3-yl}cyclopentyl (prop-2-ylamino)methanoate

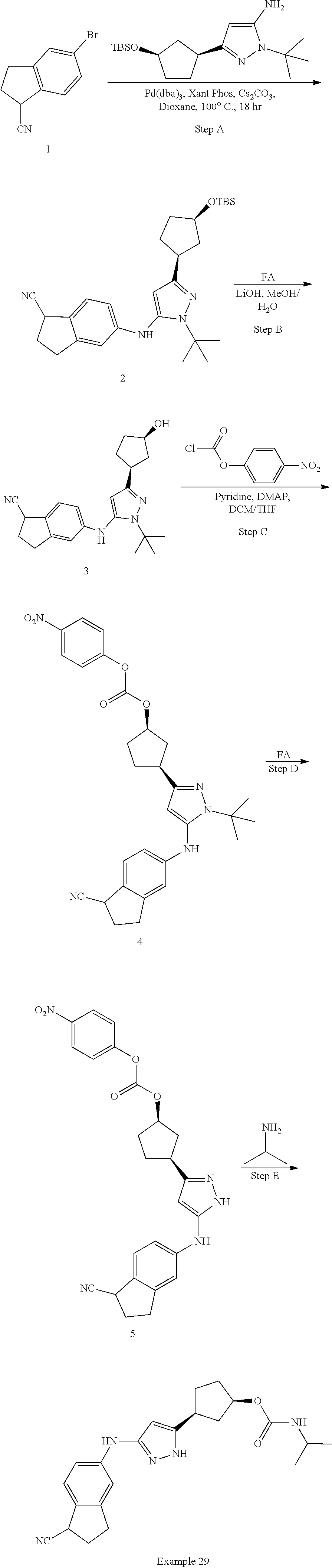

Step A: 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile To a solution of 5-bromo-2,3-dihydro-1H-indene-1-carbonitrile (200 mg, 0.901 mmol) in dioxane (10 mL) were added 5-[(1S,3R)-3-{[dimethyl(2-methylprop-2-yl)silyl]oxy}cyclopentyl]-2-(2-methylprop-2-yl)pyrazol-3-amine (304 mg, 0.901 mmol), Cs₂CO₃ (586 mg, 1.80 mmol), Xant-PHOS (104 mg, 0.180 mmol) and Pd₂(dba)₃ (82.5 mg, 0.090 mmol). The reaction mixture was stirred at 120° C. under N₂ for 3 hr. The cooled reaction mixture was diluted with EA and water. The organic layer was separated, washed with further brine, and concentrated in vacuo. The residue was purified using silica gel column chromatography eluting with ethyl acetate in petroleum ether to afford the title compound 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile (190 mg, 0.397 mmol, 44.0%) as a yellow oil. LCMS: ESI m/z 479 [M+H]⁺.

Step B: 5-({5-[(1S,3R)-3-hydroxycyclopentyl]-2-(2-methylprop-2-yl)pyrazol-3-yl}amino)-2,3-dihydro-1H-indene-1-carbonitrile A mixture of 5-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile (140 mg, 0.292 mmol) in formic acid (5 mL) was stirred at rt for 1 h and then concentrated under reduced pressure. The residue was treated with a mixture of lithium hydroxide hydrate (24.5 mg, 0.584 mmol) in MeOH (5 mL)/H₂O (1 mL). The resulting mixture was stirred at rt for 1 h and then concentrated under reduced pressure. The residue was poured into water and extracted with EtOAc. The combined organic phase was washed with brine, dried with Na₂SO₄, filtered and concentrated. The residue was purified by chromatography to give 5-({5-[(1S,3R)-3-hydroxycyclopentyl]-2-(2-methylprop-2-yl)pyrazol-3-yl}amino)-2,3-dihydro-1H-indene-1-carbonitrile (65 mg, 0.178 mmol, 60.9%) as a colorless oil. LCMS: ESI m/z 365 [M+H]⁺.

Step C: (1R,3S)-3-{5-[(1-cyano-2,3-dihydro-1H-inden-5-yl)amino]-1-(2-methylprop-2-yl)pyrazol-3-yl}cyclopentyl [(4-nitrophenyl)oxy]methanoate To a solution of 5-({5-[(1S,3R)-3-hydroxycyclopentyl]-2-(2-methylprop-2-yl)pyrazol-3-yl}amino)-2,3-dihydro-1H-indene-1-carbonitrile (80 mg, 0.219 mmol) in DCM (2 mL) and THF (2 mL) were added 4-nitrophenyl chloromethanoate (176 mg, 0.878 mmol), Py (0.053 mL, 0.657 mmol) and DMAP (2.68 mg, 0.022 mmol). The reaction mixture was stirred under at 25° C. for 2 hr. The reaction solution was diluted with EA, washed with water and brine, dried over anhydrous Na₂SO₄ and concentrated. The residue was purified by flash chromatography to afford (1R,3S)-3-{5-[(1-cyano-2,3-dihydro-1H-inden-5-yl)amino]-1-(2-methylprop-2-yl)pyrazol-3-yl}cyclopentyl [(4-nitrophenyl)oxy]methanoate (40 mg, 0.076 mmol, 34.41%) as a yellow oil. LCMS: ESI m/z 530 [M+H]⁺.

Step D: (1R,3S)-3-{5-[(1-cyano-2,3-dihydro-1H-inden-5-yl)amino]-1H-pyrazol-3-yl}cyclopentyl [(4-nitrophenyl)oxy]methanoate A solution of (1R,3S)-3-{5-[(1-cyano-2,3-dihydro-1H-inden-5-yl)amino]-1-(2-methylprop-2-yl)pyrazol-3-yl}cyclopentyl [(4-nitrophenyl)oxy]methanoate (40 mg, 0.076 mmol) in formic acid (5 mL) was stirred at 100° C. for 48 hr. The cooled reaction mixture was concentrated to afford (1R,3S)-3-{5-[(1-cyano-2,3-dihydro-1H-inden-5-yl)amino]-1H-pyrazol-3-yl}cyclopentyl [(4-nitrophenyl)oxy]methanoate (25 mg, 0.053 mmol, 69.9%) as a yellow oil. LCMS: ESI m/z 474 [M+H]⁺.

Step E: (1R,3S)-3-{5-[(1-cyano-2,3-dihydro-1H-inden-5-yl)amino]-2H-pyrazol-3-yl}cyclopentyl (prop-2-ylamino)methanoate To a solution of (1R,3S)-3-{5-[(1-cyano-2,3-dihydro-1H-inden-5-yl)amino]-1H-pyrazol-3-yl}cyclopentyl [(4-nitrophenyl)oxy]methanoate (25 mg, 0.053 mmol) in THF (3 mL) was added propan-2-amine (0.014 mL, 0.158 mmol) and the reaction was stirred at rt for 1 h. The reaction concentrated in vacuo. The residue was purified by prep-HPLC to afford the title compound (1R,3S)-3-{5-[(1-cyano-2,3-dihydro-1H-inden-5-yl)amino]-2H-pyrazol-3-yl}cyclopentyl (prop-2-ylamino)methanoate (1.5 mg, 0.004 mmol, 7.22%) as a white solid. LCMS: ESI m/z 394 [M+H]⁺. ¹H NMR (400 MHz, DMSO) δ 11.68 (s, 1H), 8.34 (s, 1H), 7.33 (s, 1H), 7.15 (s, 2H), 6.95 (d, J=6.8 Hz, 1H), 5.61 (s, 1H), 4.98 (s, 1H), 4.26 (t, J=7.6 Hz, 1H), 3.64-3.53 (m, 1H), 3.08-2.98 (m, 1H), 2.98-2.77 (m, 2H), 2.49-2.41 (m, 2H), 2.27-2.13 (m, 1H), 2.05-1.95 (m, 1H), 1.94-1.83 (m, 1H), 1.75-1.52 (m, 3H), 1.03 (d, J=6.4 Hz, 6H).

### Example 30: (1R,3S)-3-{5-[(2-oxo-1,2,3,4-tetrahydroquinolin-6-yl)amino]-1H-pyrazol-3-yl}cyclopentyl (prop-2-ylamino)methanoate

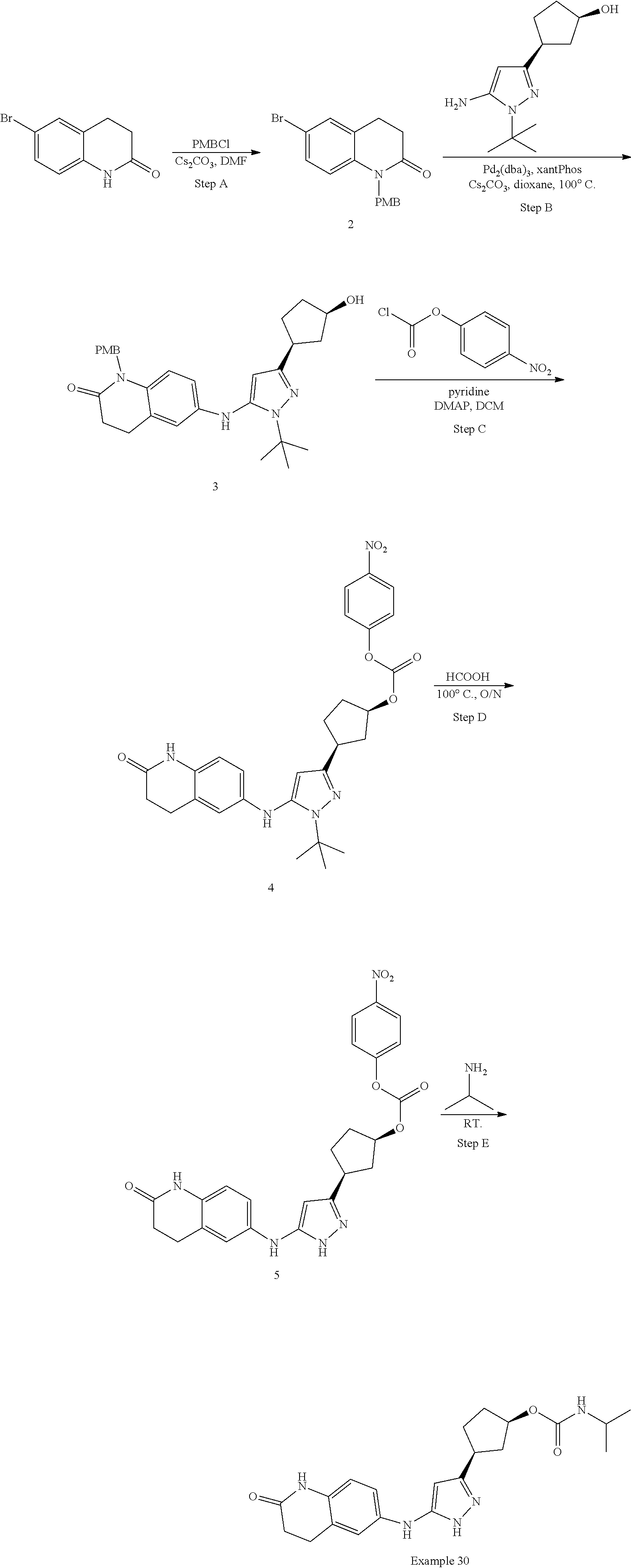

Step A: 6-bromo-1-[(4-methoxyphenyl)methyl]-1,2,3,4-tetrahydroquinolin-2-one To a solution of 6-bromo-1,2,3,4-tetrahydroquinolin-2-one (1000 mg, 4.42 mmol) in DMF (10 mL) were added Cs₂CO₃ (2.88 g, 8.84 mmol), 1-(chloromethyl)-4-methoxybenzene (0.9 mL, 6.63 mmol) and the reaction mixture was stirred 50° C. for 2 hr. The cooled reaction mixture was diluted with H₂O and extracted with EA. The organic layer was washed with brine, dried by Na₂SO₄, filtered and concentrated. The residue was purified using silica gel column chromatography eluting with EA in PE [Gradient: 0-50%] afford the title compound 6-bromo-1-[(4-methoxyphenyl)methyl]-1,2,3,4-tetrahydroquinolin-2-one (1.17 g, 3.37 mmol, 76.4%) as a white solid. LCMS: ESI m/z 348 [M+H]⁺.

Step B: 6-({5-[(1S,3R)-3-hydroxycyclopentyl]-2-(2-methylprop-2-yl)pyrazol-3-yl}amino)-1-[(4-methoxyphenyl)methyl]-1,2,3,4-tetrahydroquinolin-2-one To a solution of (1R,3S)-3-[5-amino-1-(2-methylprop-2-yl) pyrazol-3-yl]cyclopentan-1-ol (500 mg, 2.24 mmol) in dioxane (10 mL) were added Cs₂CO₃ (1.45 g, 4.48 mmol), 6-bromo-1-[(4-methoxyphenyl)methyl]-1,2,3,4-tetrahydroquinolin-2-one (1.16 g, 3.36 mmol), Pd₂(dba)₃ (205 mg, 0.22 mmol) and Xant-PHOS (259 mg, 0.45 mmol). The reaction mixture was stirred at 100° C. under N₂ overnight. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 0-100%] to afford the title compound 6-({5-[(1S,3R)-3-hydroxycyclopentyl]-2-(2-methylprop-2-yl)pyrazol-3-yl}amino)-1-[(4-methoxyphenyl)methyl]-1,2,3,4-tetrahydroquinolin-2-one (200 mg, 0.41 mmol, 18.2%) as a white solid. LCMS: ESI m/z 489 [M+H]⁺.

Step C: (1R,3S)-3-[5-({1-[(4-methoxyphenyl)methyl]-2-oxo-1,2,3,4-tetrahydroquinolin-6-yl}amino)-1-(2-methylprop-2-yl)pyrazol-3-yl]cyclopentyl [(4-nitrophenyl)oxy]methanoate To a solution of 6-({5-[(1S,3R)-3-hydroxycyclopentyl]-2-(2-methylprop-2-yl)pyrazol-3-yl}amino)-1-[(4-methoxyphenyl)methyl]-1,2,3,4-tetrahydroquinolin-2-one (150 mg, 0.307 mmol) in DCM (5 mL) were added 4-nitrophenyl chloromethanoate (92.8 mg, 0.460 mmol), pyridine (0.050 mL, 0.614 mmol) and DMAP (3.75 mg, 0.031 mmol). The reaction mixture was stirred at 40° C. for 4 h and then concentrated. The residue was purified using silica gel column chromatography eluting with EA in PE [Gradient: 0-100%] to afford the compound (1R,3S)-3-[5-({1-[(4-methoxyphenyl)methyl]-2-oxo-1,2,3,4-tetrahydroquinolin-6-yl}amino)-1-(2-methylprop-2-yl)pyrazol-3-yl]cyclopentyl [(4-nitrophenyl)oxy]methanoate (125 mg, 0.191 mmol, 62.2%) as a white solid. LCMS: ESI m/z 654 [M+H]⁺.

Step D: (1R,3S)-3-{5-[(2-oxo-1,2,3,4-tetrahydroquinolin-6-yl) amino]-1H-pyrazol-3-yl}cyclopentyl [(4-nitrophenyl)oxy]methanoate A solution of (1R,3S)-3-[5-({1-[(4-methoxyphenyl) methyl]-2-oxo-1,2,3,4-tetrahydroquinolin-6-yl)amino)-1-(2-methylprop-2-yl)pyrazol-3-yl]cyclopentyl [(4-nitrophenyl)oxy]methanoate (125 mg, 0.191 mmol) in HCOOH (3 mL) was stirred at 100° C. overnight. The cooled reaction mixture was concentrated in vacuo to afford the compound (1R,3S)-3-{5-[(2-oxo-1,2,3,4-tetrahydroquinolin-6-yl)amino]-1H-pyrazol-3-yl}cyclopentyl [(4-nitrophenyl)oxy]methanoate (100 mg crude) as a white solid. LCMS: ESI m/z 478 [M+H]⁺.

Step E: (1R,3S)-3-{5-[(2-oxo-1,2,3,4-tetrahydroquinolin-6-yl)amino]-1H-pyrazol-3-yl}cyclopentyl (prop-2-ylamino)methanoate A solution of (1R,3S)-3-{5-[(2-oxo-1,2,3,4-tetrahydroquinolin-6-yl)amino]-1H-pyrazol-3-yl}cyclopentyl [(4-nitrophenyl)oxy]methanoate (125 mg, 0.262 mmol) in propan-2-amine (3 mL) was stirred RT for 4 h. The reaction mixture was concentrated. The residue was purified by prep-HPLC to afford the title compound (1R,3S)-3-{5-[(2-oxo-1,2,3,4-tetrahydroquinolin-6-yl)amino]-1H-pyrazol-3-yl}cyclopentyl (prop-2-ylamino)methanoate (2 mg, 0.075 mmol, 2.88%) as a white solid. LCMS: ESI m/z 398 [M+H]⁺.

¹H NMR (400 MHz, DMSO) δ 9.86 (s, 1H), 8.12 (s, 1H), 7.24 (s, 1H), 7.14 (d, J=8.4 Hz, 1H), 7.02 (s, 1H), 6.75 (d, J=8.4 Hz, 1H), 5.64 (s, 1H), 5.07 (s, 1H), 3.15-3.05 (m, 2H), 2.86 (t, J=7.2 Hz, 2H), 2.55-2.50 (m, 1H), 2.49-2.44 (m, 2H), 2.11-2.05 (m, 1H), 2.04-1.94 (m, 1H), 1.85-1.70 (m, 2H), 1.70-1.61 (m, 1H), 1.11 (d, J=6.4 Hz, 6H).

### Example 31: (1R,3S)-3-{5-[(3-cyano-2,3-dihydro-1H-inden-5-yl)amino]-2H-pyrazol-3-yl}cyclopentyl (prop-2-ylamino)methanoate

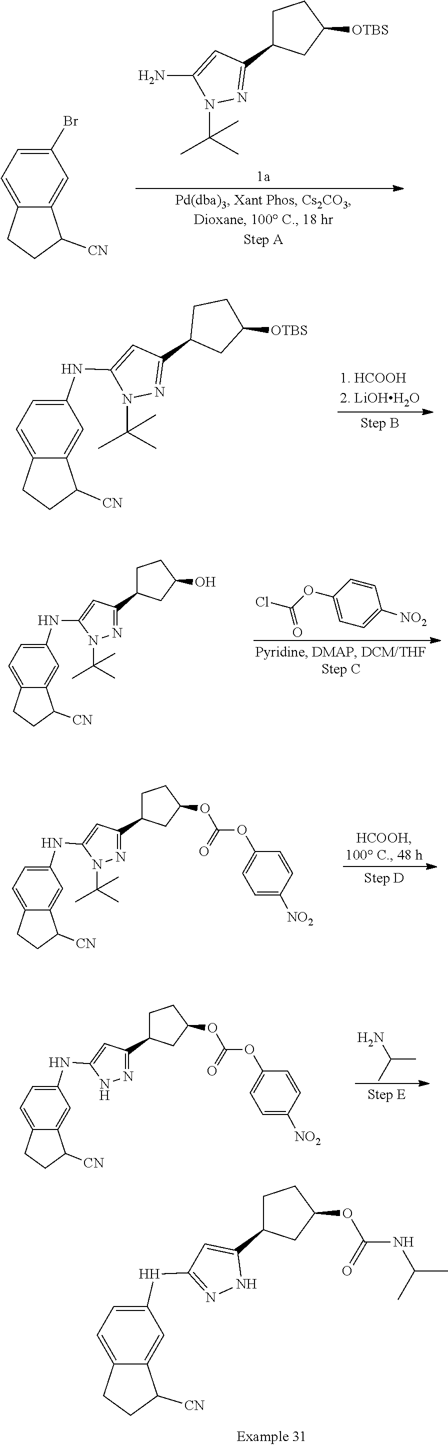

Step A: 6-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile To a stirred mixture of (1R,3S)-3-[5-amino-1-(2-methylprop-2-yl)pyrazol-3-yl]cyclopentan-1-ol (20 mg, 0.09 mmol) in dioxane (15 mL) was added 5-bromo-1-methyl-2,3-dihydro-1H-indol-2-one (26.3 mg, 0.116 mmol), Xant Phos (103 mg, 0.178 mmol), Cs₂CO₃ (579 mg, 1.78 mmol) and Pd₂(dba)₃ (81.38 mg, 0.089 mmol). The reaction mixture was stirred at 100° C. under N₂ for 18 hr. The reaction was complete detected by LCMS. The residue was dissolved in EA (5 mL*2), washed with H₂O (10 mL) and brine, dried over sodium sulfate and concentrated. The residue was purified by silica gel chromatography (eluting with 50% ethyl acetate in petroleum ether) to afford 6-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile (340 mg, 0.497 mmol, 55.9%) as a yellow oil. LCMS: ESI m/z 479.6 [M+H]⁺.

Step B: 6-({5-[(1S,3R)-3-hydroxycyclopentyl]-2-(2-methylprop-2-yl)pyrazol-3-yl}amino)-2,3-dihydro-1H-indene-1-carbonitrile A solution of 6-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1H-indene-1-carbonitrile (340 mg, 0.497 mmol) in HCOOH (5 mL) was stirred at rt for 2 hr. The reaction mixture was concentrated in vacuo. The residue was dissolved with methanol and adjusted to pH value 12-13 with aq.2 N lithium hydroxide aqueous solution. The reaction mixture was stirred at rt for 1 h and then diluted with EA and water. The organic layer was separated, washed with brine, dried over Na₂SO₄ and concentrated. The residue was purified using silica gel column chromatography eluted with MeOH and 1% NH₄0H in DCM (gradient:0-10%) to afford the title compound 6-({5-[(1S,3R)-3-hydroxycyclopentyl]-2-(2-methylprop-2-yl)pyrazol-3-yl}amino)-2,3-dihydro-1H-indene-1-carbonitrile (150 mg, 0.412 mmol, 82.8%) as a yellow oil. LCMS: 365.2 [M+H]⁺.

Step C: (1R,3S)-3-{5-[(3-cyano-2,3-dihydro-1H-inden-5-yl)amino]-1-(2-methylprop-2-yl)pyrazol-3-yl}cyclopentyl [(4-nitrophenyl)oxy]methanoate A suspension of 6-({5-[(1S,3R)-3-hydroxycyclopentyl]-2-(2-methylprop-2-yl)pyrazol-3-yl}amino)-2,3-dihydro-1H-indene-1-carbonitrile (100 mg, 0.274 mmol), pyridine (6 mL, 74.2 mmol), DMAP (3.35 mg, 0.027 mmol) and 4-nitrophenyl chloromethanoate (277 mg, 1.372 mmol) in THF (3 mL) and DCM (3 mL) was stirred at rt overnight. The solvents were removed under vacuum. The residue was dissolved in ethyl acetate (50 mL*2), washed with brine, dried over sodium sulfate and concentrated. The residue was purified by silica gel chromatography (eluting with 100% ethyl acetate in petroleum ether) to afford (1R,3S)-3-{5-[(3-cyano-2,3-dihydro-1H-inden-5-yl)amino]-1-(2-methylprop-2-yl)pyrazol-3-yl}cyclopentyl[(4-nitrophenyl)oxy]methanoate (65 mg, 0.123 mmol, 44.7%) as a white solid. LCMS: 530.4 [M+H]⁺.

Step D: (1R,3S)-3-{5-[(3-cyano-2,3-dihydro-1H-inden-5-yl)amino]-1H-pyrazol-3-yl}cyclopentyl [(4-nitrophenyl)oxy]methanoate A solution of (1R,3S)-3-{5-[(3-cyano-2,3-dihydro-1H-inden-5-yl)amino]-1-(2-methylprop-2-yl)pyrazol-3-yl}cyclopentyl [(4-nitrophenyl)oxy]methanoate (80 mg, 0.151 mmol) in HCOOH (8 mL) was stirred at 100° C. for 18 hr. The reaction was concentrated in vacuo to afford the title compound (1R,3S)-3-{5-[(3-cyano-2,3-dihydro-1H-inden-5-yl)amino]-1H-pyrazol-3-yl}cyclopentyl[(4-nitrophenyl)oxy]methanoate (50 mg, 0.106 mmol, 69.9%). LCMS: 474.3 [M+H]⁺

Step E: (1R,3S)-3-{5-[(3-cyano-2,3-dihydro-1H-inden-5-yl)amino]-2H-pyrazol-3-yl}cyclopentyl (prop-2-ylamino)methanoate A solution of (1R,3S)-3-{5-[(3-cyano-2,3-dihydro-1H-inden-5-yl)amino]-1H-pyrazol-3-yl}cyclopentyl [(4-nitrophenyl)oxy]methanoate (50 mg, 0.106 mmol) in propan-2-amine (1.81 mL, 21.1 mmol) was stirred at room temperature for 1 hr. The reaction mixture was concentrated and residue was purified by prep-HPLC (C18, 5%-95% MeCN in H₂O with 0.1% HCOOH) to give (1R,3S)-3-{5-[(3-cyano-2,3-dihydro-1H-inden-5-yl)amino]-2H-pyrazol-3-yl}cyclopentyl (prop-2-ylamino)methanoate (6 mg, 0.015 mmol, 14.4%) as a white solid. LCMS: m/z 394.1 [M+H]⁺. ¹H NMR (400 MHz, MeOD) δ 7.27 (d, J=8.4 Hz, 2H), 7.12 (d, J=8.4 Hz, 1H), 5.10 (s, 1H), 4.28 (t, J=8.0 Hz, 1H), 3.72-3.62 (m, 1H), 3.25-3.15 (m, 1H), 3.08-2.99 (m, 1H), 2.94 (dt, J=16.0, 8.0 Hz, 1H), 2.66-2.51 (m, 2H), 2.31 (dq, J=12.8, 8.0 Hz, 1H), 2.23-2.13 (m, 1H), 2.00-1.84 (m, 3H), 1.31 (d, J=18.4 Hz, 2H), 1.15-1.02 (m, 6H).

### Example 32: (1R,3S)-3-(3-((2,2-difluoro-2,3-dihydro-1H-inden-5-yl)amino)-1H-pyrazol-5-yl)cyclopentyl isopropylcarbamate

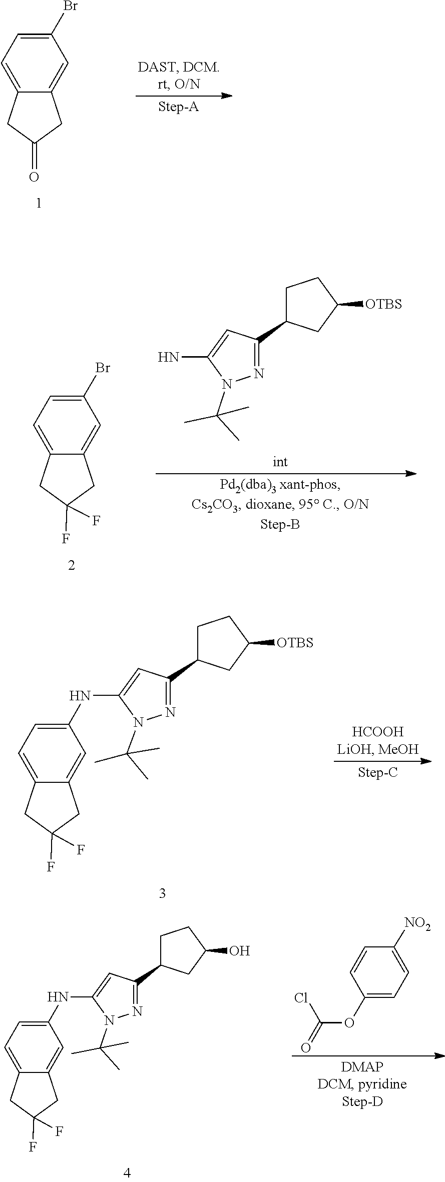 [SMILES: O=C1Cc2ccc(Br)cc2C1 ; FC1(F)Cc2ccc(Br)cc2C1 ; CC(C)(C)n1nc([C@H]2CC[C@@H](O[Si](C)(C)C(C)(C)C)C2)cc1N ; CC(C)(C)n1nc([C@H]2CC[C@@H](O[Si](C)(C)C(C)(C)C)C2)cc1Nc1ccc2c(c1)CC(F)(F)C2 ; CC(C)(C)n1nc([C@H]2CC[C@@H](O)C2)cc1Nc1ccc2c(c1)CC(F)(F)C2 ; O=C(Cl)Oc1ccc([N+](=O)[O-])cc1]

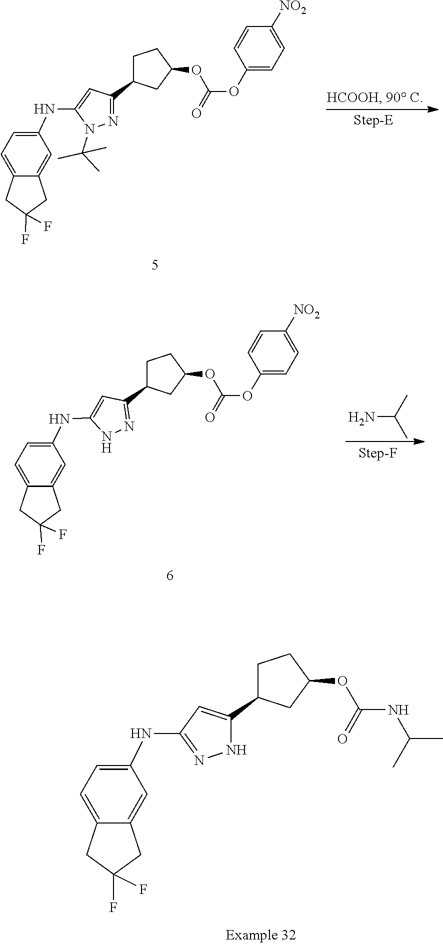

Step A: 5-bromo-2,2-difluoro-2,3-dihydro-1H-indene To a solution of 5-bromo-2,3-dihydro-1H-inden-2-one (500 mg, 2.37 mmol) in DCM (5 mL) was added diethyl(trifluoro-λ⁴-sulfanyl)amine (1.6 ml, 11.85 mmol). The reaction mixture was stirred at rt overnight. The reaction mixture was diluted with DCM and water. The organic layer was separated, washed with brine and concentrated in vacuo. The residue was purified using silica gel column chromatography (eluting with 10% ethyl acetate in petroleum ether) to afford the title compound 5-bromo-2,2-difluoro-2,3-dihydro-1H-indene (150 mg, 0.65 mmol, 27.2%) as colorless oil. LCMS: 233/235 [M+H]⁺.

Step B: 1-(tert-butyl)-3-((1S,3R)-3-((tert-butyldimethylsilyl)oxy)cyclopentyl)-N-(2,2-difluoro-2,3-dihydro-1H-inden-5-yl)-1H-pyrazol-5-amine To a solution of 1-(tert-butyl)-3-((1S,3R)-3-((tert-butyldimethylsilyl)oxy)cyclopentyl)-1H-pyrazol-5-amine (70 mg, 0.20 mmol) in dioxane (5 mL) was added N-(5-bromo-2-hydroxyphenyl)-4-methylbenzenesulfonamide (41 mg, 0.12 mmol), Pd₂(dba)₃ (19 mg, 0.02 mmol), Xant-PHOS (12.00 mg, 0.021 mmol) and Cs₂CO₃ (168 mg, 0.52 mmol). The reaction mixture was stirred at 95° C. under N₂ atmosphere overnight. The cooled reaction mixture was diluted with EA and water. The organic layer was separated, washed with brine and concentrated in vacuo. The residue was purified using silica gel column chromatography (eluting with 50% ethyl acetate in petroleum ether) to afford the title compound 1-(tert-butyl)-3-((1S,3R)-3-((tert-butyldimethylsilyl)oxy)cyclopentyl)-N-(2,2-difluoro-2,3-dihydro-1H-inden-5-yl)-1H-pyrazol-5-amine (100 mg, 0.204 mmol, 98.5%) as yellow oil. LCMS: 490 [M+H]⁺.

Step C: (1R,3S)-3-(1-(tert-butyl)-5-((2,2-difluoro-2,3-dihydro-1H-inden-5-yl)amino)-1H-pyrazol-3-yl)cyclopentan-1-ol A solution of 1-(tert-butyl)-3-((1S,3R)-3-((tert-butyldimethylsilyl)oxy)cyclopentyl)-N-(2,2-difluoro-2,3-dihydro-1H-inden-5-yl)-1H-pyrazol-5-amine (100 mg, 0.20 mmol) in formic acid (3 mL) was stirred at RT for 2 hours and then concentrated in vacuo. The residue was added MeOH (3 mL)/Water (3 mL) and LiOH (0.13 mL, 4.77 mmol). The reaction mixture was stirred at rt for 1 h and then diluted with EA and water. The organic layer was separated, washed with brine, and concentrated in vacuo. The residue was purified using silica gel column chromatography (eluting with 50% ethyl acetate in petroleum ether) to afford the title compound (1R,3S)-3-(1-(tert-butyl)-5-((2,2-difluoro-2,3-dihydro-1H-inden-5-yl)amino)-1H-pyrazol-3-yl)cyclopentan-1-ol (60 mg, 0.096 mmol, 46.9%) as yellow oil. LCMS: 376 [M+H]⁺.

Step D: (1R,3S)-3-(1-(tert-butyl)-5-((2,2-difluoro-2,3-dihydro-1H-inden-5-yl)amino)-1H-pyrazol-3-yl)cyclopentyl (4-nitrophenyl) carbonate To a solution of (1R,3S)-3-(1-(tert-butyl)-5-((2,2-difluoro-2,3-dihydro-1H-inden-5-yl)amino)-1H-pyrazol-3-yl)cyclopentan-1-ol (60 mg, 0.10 mmol) in THF (2 mL)/DCM (2 mL) was added DMAP (2 mg, 0.010 mmol)/Pyridine (0.025 mL, 0.3 mmol) and 4-nitrophenyl chloromethanoate (194 mg, 0.96 mmol). The reaction mixture was stirred at rt overnight. The reaction mixture was diluted with EA and water. The organic layer was separated, washed with brine and concentrated in vacuo. The residue was purified using silica gel column chromatography (eluting with 50% ethyl acetate in petroleum ether) to afford the title compound (1R,3S)-3-(1-(tert-butyl)-5-((2,2-difluoro-2,3-dihydro-1H-inden-5-yl)amino)-1H-pyrazol-3-yl)cyclopentyl (4-nitrophenyl) carbonate (30 mg, 0.045 mmol, 46.3%) as yellow oil. LCMS: 571 [M+H]⁺.

Step E: (1R,3S)-3-(5-((2,2-difluoro-2,3-dihydro-1H-inden-5-yl)amino)-1H-pyrazol-3-yl)cyclopentyl (4-nitrophenyl) carbonate A solution of (1R,3S)-3-(1-(tert-butyl)-5-((2,2-difluoro-2,3-dihydro-1H-inden-5-yl)amino)-1H-pyrazol-3-yl)cyclopentyl (4-nitrophenyl) carbonate (30 mg, 0.055 mmol) in formic acid (3 mL) was stirred at 100° C. overnight. The cooled reaction mixture was concentrated to afford the title compound (1R,3S)-3-(5-((2,2-difluoro-2,3-dihydro-1H-inden-5-yl)amino)-1H-pyrazol-3-yl)cyclopentyl (4-nitrophenyl) carbonate (20 mg, 0.03 mmol, 52.0%) as yellow oil. LCMS: 485 [M+H]⁺.

Step F: (1R,3S)-3-(3-((2,2-difluoro-2,3-dihydro-1H-inden-5-yl)amino)-1H-pyrazol-5-yl)cyclopentyl isopropylcarbamateA solution of (1R,3S)-3-(5-((2,2-difluoro-2,3-dihydro-1H-inden-5-yl)amino)-1H-pyrazol-3-yl)cyclopentyl (4-nitrophenyl) carbonate (20 mg, 0.04 mmol) in propan-2-amine (2 mL, 23.4 mmol) was stirred at rt for 2 hours. LCMS showed less SM and DP. The reaction was diluted with EA and water. The organic layer was separated, washed with brine and concentrated in vacuo. The residue was purified by prep-HPLC and dried to afford the title compound (1R,3S)-3-(3-((2,2-difluoro-2,3-dihydro-1H-inden-5-yl)amino)-1H-pyrazol-5-yl)cyclopentyl isopropylcarbamate (3 mg, 0.007 mmol, 18.0%) as yellow oil. LCMS: 405 [M+H]⁺. ¹H NMR (400 MHz, DMSO) δ 8.26 (s, 1H), 7.29 (s, 1H), 7.10 (d, J=8.4 Hz, 1H), 7.03 (d, J=8.4 Hz, 1H), 6.95 (d, J=7.6 Hz, 1H), 5.60 (s, 1H), 4.99 (s, 1H), 3.61-3.51 (m, 1H), 3.27 (s, 1H), 3.09-2.95 (m, 1H), 2.47-2.37 (m, 1H), 2.08-1.95 (m, 2H), 1.95-1.87 (m, 1H), 1.74-1.55 (m, 3H), 1.24 (s, 2H), 1.03 (d, J=6.4 Hz, 6H).

### Example 33: (1s,4s)-4-(3-((2,2-dioxido-1,3-dihydrobenzo[c]thiophen-5-yl)amino)-1H-pyrazol-5-yl)cyclohexyl isopropylcarbamate

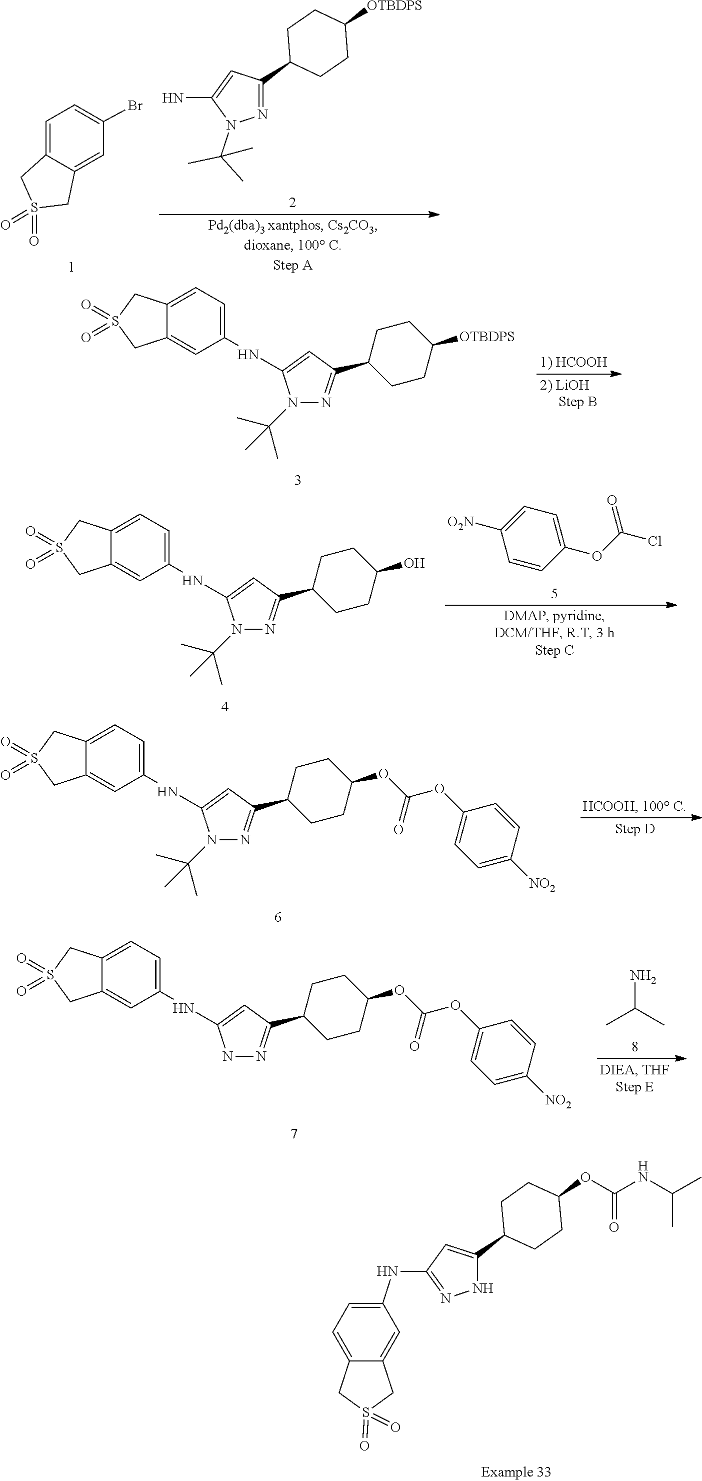

Step A: 5-((1-(tert-butyl)-3-((1s,4s)-4-((tert-butyldiphenylsilyl)oxy)cyclohexyl)-1H-pyrazol-5-yl)amino)-1,3-dihydrobenzo[c]thiophene 2,2-dioxide To a solution of 5-bromo-1,3-dihydrobenzo[c]thiophene 2,2-dioxide (450 mg, 1.82 mmol) in dioxane (15 mL) was added 1-(tert-butyl)-3-((1s,4s)-4-((tert-butyldiphenylsilyl)oxy)cyclohexyl)-1H-pyrazol-5-amine (250 mg, 0.525 mmol), Cs₂CO₃ (514 mg, 1.58 mmol), Pd₂(dba)₃ (48.1 mg, 0.0530 mmol) and Xantphos (60.8 mg, 0.105 mmol). The reaction mixture was stirred at 100° C. under N₂ atmosphere for 1 hr. The reaction was complete detected by LCMS. The cooled reaction mixture was dissolved in EA (30 mL), washed with H₂O (20 mL) and brine, dried over sodium sulfate and concentrated. The residue was purified by silica gel chromatography (eluting with 20-80% EA in PE) to afford 5-((1-(tert-butyl)-3-((1s,4s)-4-((tert-butyldiphenylsilyl)oxy)cyclohexyl)-1H-pyrazol-5-yl)amino)-1,3-dihydrobenzo[c]thiophene 2,2-dioxide (300 mg, 0.467 mmol, 88.9%) as a yellow solid. LCMS: ESI m/z 642.95 [M+H]⁺.

Step B: 5-((1-(tert-butyl)-3-((1s,4s)-4-hydroxycyclohexyl)-1H-pyrazol-5-yl)amino)-1,3-dihydrobenzo[c]thiophene 2,2-dioxide A solution of 5-((1-(tert-butyl)-3-((1s,4s)-4-((tert-butyldiphenylsilyl)oxy)cyclohexyl)-1H-pyrazol-5-yl)amino)-1,3-dihydrobenzo[c]thiophene 2,2-dioxide (300 mg, 0.467 mmol) in formic acid (3 mL) was stirred at room temperature overnight. The reaction was complete detected by LCMS. The solvents were removed under vacuum. Then add EtOH (2 mL), H₂O (2 mL) and Lithium Hydroxide Monohydrate (58.8 mg, 1.40 mmol) to alkaline pH. The mixture was stirred at room temperature for 1 hr. LCMS showed the reaction was completed. The reaction mixture was dissolved in EA (50 mL), washed with H₂O (10 mL) and brine, dried over sodium sulfate and concentrated. The residue was purified by silica gel chromatography (eluting with 0-12% MeOH in DCM) to afford 5-((1-(tert-butyl)-3-((1s,4s)-4-hydroxycyclohexyl)-1H-pyrazol-5-yl)amino)-1,3-dihydrobenzo[c]thiophene 2,2-dioxide (100 mg, 0.248 mmol, 53.0%) as a white solid. LCMS: ESI m/z 404.54 [M+H]⁺.

Step C: (1s,4s)-4-(1-(tert-butyl)-5-((2,2-dioxido-1,3-dihydrobenzo[c]thiophen-5-yl)amino)-1H-pyrazol-3-yl)cyclohexyl (4-nitrophenyl) carbonate A suspension of 5-((1-(tert-butyl)-3-((1s,4s)-4-hydroxycyclohexyl)-1H-pyrazol-5-yl)amino)-1,3-dihydrobenzo[c]thiophene 2,2-dioxide (100 mg, 0.248 mmol), DMAP (3.05 mg, 0.0250 mmol), py (0.0600 mL, 0.743 mmol) and 4-nitrophenyl chloromethanoate (250 mg, 1.24 mmol) in DCM (3 mL) and THF (3 mL) was stirred at room temperature overnight. The reaction was completed by LCMS. The solvents were removed. The residue was dissolved in EA (50 mL), washed with H₂O (5 mL) and brine, dried over sodium sulfate and concentrated. The residue was purified by silica gel chromatography (eluting with 0-12% MeOH in DCM) to afford (1s,4s)-4-(1-(tert-butyl)-5-((2,2-dioxido-1,3-dihydrobenzo[c]thiophen-5-yl)amino)-1H-pyrazol-3-yl)cyclohexyl (4-nitrophenyl) carbonate (45.0 mg, 0.0790 mmol, 31.9%) as a white solid. LCMS: ESI m/z 569.64 [M+H]⁺.

Step D: (1s,4s)-4-(5-((2,2-dioxido-1,3-dihydrobenzo[c]thiophen-5-yl)amino)-1H-pyrazol-3-yl)cyclohexyl (4-nitrophenyl) carbonate A solution of (1s,4s)-4-(1-(tert-butyl)-5-((2,2-dioxido-1,3-dihydrobenzo[c]thiophen-5-yl)amino)-1H-pyrazol-3-yl)cyclohexyl (4-nitrophenyl) carbonate (45.0 mg, 0.0790 mmol) in formic acid (3 mL) was stirred at 100° C. overnight. LCMS showed the reaction was completed. The reaction mixture was concentrated to give crude (1s,4s)-4-(5-((2,2-dioxido-1,3-dihydrobenzo[c]thiophen-5-yl)amino)-1H-pyrazol-3-yl)cyclohexyl (4-nitrophenyl) carbonate (40.0 mg, 0.0780 mmol, 98.6%) as a yellow oil. LCMS: ESI m/z 513.54 [M+H]⁺.

Step E: (1s,4s)-4-(3-((2,2-dioxido-1,3-dihydrobenzo[c]thiophen-5-yl)amino)-1H-pyrazol-5-yl)cyclohexyl isopropylcarbamate To a suspension of (1s,4s)-4-(5-((2,2-dioxido-1,3-dihydrobenzo[c]thiophen-5-yl)amino)-1H-pyrazol-3-yl)cyclohexyl (4-nitrophenyl) carbonate (40.0 mg, 0.0780 mmol) in THF (2 mL) was added propan-2-amine (0.0330 mL, 0.390 mmol) and DIEA (0.0390 mL, 0.234 mmol). The reaction mixture was stirred at room temperature for 3 hrs. LCMS showed the reaction had been completed. The reaction mixture was dissolved in EA (50 mL), washed with H₂O (10 mL) and brine, dried over sodium sulfate and concentrated. The residue was purified by prep-HPLC (C18, 15˜ 95% MeCN in H₂O with 0.1% FA) to afford (1s,4s)-4-(3-((2,2-dioxido-1,3-dihydrobenzo[c]thiophen-5-yl)amino)-1H-pyrazol-5-yl)cyclohexyl isopropylcarbamate (2.00 mg, 0.00500 mmol, 5.92%) as a yellow solid. LCMS: 433.54 [M+H]⁺. ¹H NMR (400 MHz, DMSO) δ 7.43 (s, 1H), 7.30-7.20 (m, 2H), 6.94 (d, J=6.4 Hz, 1H), 5.77 (s, 1H), 4.81 (s, 1H), 4.48 (s, 2H), 4.40 (s, 2H), 3.68-3.62 (m, 1H), 2.75-2.65 (m, 1H), 1.89-1.65 (m, 8H), 1.10 (d, J=6.4 Hz, 6H).

### Example 34: (1R,3S)-3-{5-[(3-methyl-2-oxobenzo[d][1,3]oxazol-6-yl)amino]-2H-pyrazol-3-yl}cyclopentyl (prop-2-ylamino)methanoate

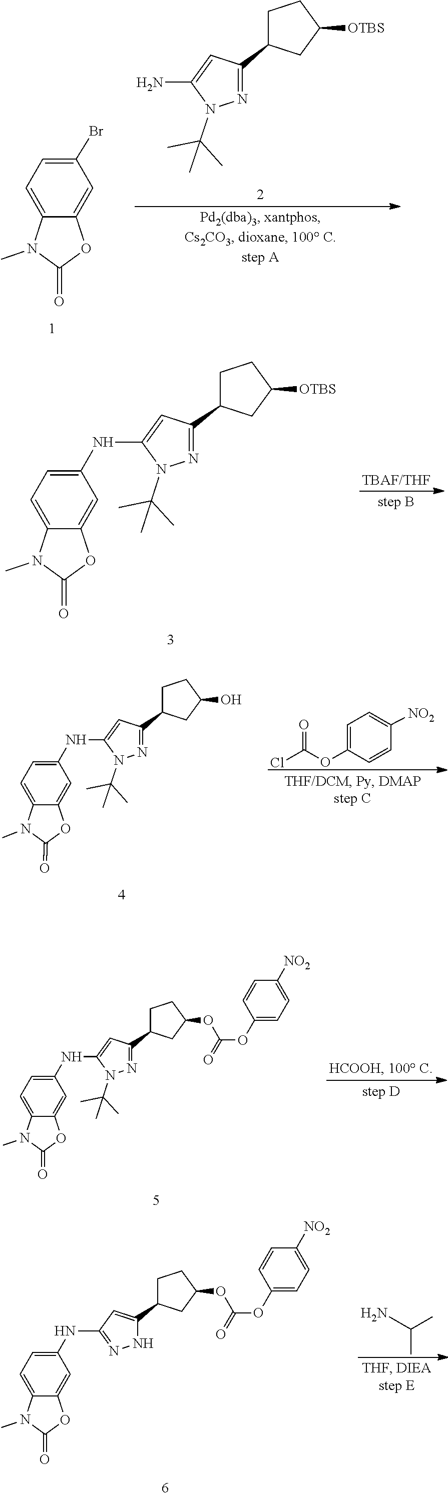

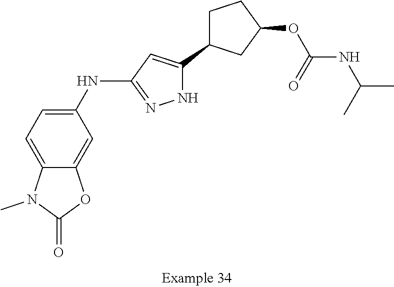 [SMILES: CC(C)NC(=O)O[C@@H]1CC[C@H](c2cc(Nc3ccc4c(c3)oc(=O)n4C)n[nH]2)C1]

Step A: 6-({5-[(1S,3R)-3-{[dimethyl(2-methylprop-2-yl)silyl]oxy}cyclopentyl]-1-(2-methylprop-2-yl)pyrazol-3-yl}amino)-3-methyl-2,3-dihydrobenzo[d][1,3]oxazol-2-one To a solution of 6-bromo-3-methyl-2,3-dihydrobenzo[d][1,3]oxazol-2-one (432 mg, 1.90 mmol) in dioxane (15 mL) was added 5-[(1S,3R)-3-{[dimethyl(2-methylprop-2-yl)silyl]oxy}cyclopentyl]-2-(2-methylprop-2-yl)pyrazol-3-amine (320 mg, 0.948 mmol), Pd₂(dba)₃ (86.8 mg, 0.095 mmol), xant-PHOS (54.8 mg, 0.095 mmol) and Cs₂CO₃ (772 mg, 2.37 mmol). The reaction mixture was purged with N₂ and stirred at 95° C. under N₂ for 4 h. The cooled reaction mixture was diluted with EA and water. The organic layer was separated, washed with further brine, and concentrated in vacuo. The residue was purified by chromatography (silica gel, 0-60%, EtOAc in PE) to give crude 6-({5-[(1S,3R)-3-{[dimethyl(2-methylprop-2-yl)silyl]oxy}cyclopentyl]-1-(2-methylprop-2-yl)pyrazol-3-yl}amino)-3-methyl-2,3-dihydrobenzo[d][1,3]oxazol-2-one (440 mg, 0.908 mmol, 95.8%) as a yellow solid. LCMS: ESI m/z 485 [M+H]⁺.

Step B: 6-({3-[(1S,3R)-3-hydroxycyclopentyl]-1-(2-methylprop-2-yl)pyrazol-5-yl}amino)-3-methyl-2,3-dihydrobenzo[d][1,3]oxazol-2-one A solution of 6-({3-[(1S,3R)-3-{[dimethyl(2-methylprop-2-yl)silyl]oxy}cyclopentyl]-1-(2-methylprop-2-yl)pyrazol-5-yl}amino)-3-methyl-2,3-dihydrobenzo[d][1,3]oxazol-2-one (400 mg, 0.825 mmol) in TBAF-THF (5 mL, 1M in THF) was stirred at room temperature for 4 hrs. The reaction mixture was concentrated and the residue was purified by silica gel column chromatography eluted with ethyl acetate in petroleum ether (gradient: 0-50%) to afford the title compound 6-({3-[(1S,3R)-3-hydroxycyclopentyl]-1-(2-methylprop-2-yl)pyrazol-5-yl}amino)-3-methyl-2,3-dihydrobenzo[d][1,3]oxazol-2-one (210 mg, 0.567 mmol, 68.7%) as a purple solid. LCMS: m/z 371 [M+H]⁺.

Step C: (1R,3S)-3-{5-[(3-methyl-2-oxobenzo[d][1,3]oxazol-6-yl)amino]-1-(2-methylprop-2-yl)pyrazol-3-yl}cyclopentyl [(4-nitrophenyl)oxy]methanoate To a solution of 6-({3-[(1S,3R)-3-hydroxycyclopentyl]-1-(2-methylprop-2-yl)pyrazol-5-yl}amino)-3-methyl-2,3-dihydrobenzo[d][1,3]oxazol-2-one (190 mg, 0.513 mmol) in DCM (3 mL) and THF (3 mL) was added 4-nitrophenyl chloromethanoate (310 mg, 1.54 mmol), DMAP (6.27 mg, 0.051 mmol) and pyridine (0.124 mL, 1.54 mmol). The reaction mixture was stirred at rt for 3 h. The reaction mixture was concentrated in vacuo. The residue was purified using silica gel column chromatography eluting with EA in PE [gradient: 0-50%] to afford the title compound (1R,3S)-3-{5-[(3-methyl-2-oxobenzo[d][1,3]oxazol-6-yl)amino]-1-(2-methylprop-2-yl)pyrazol-3-yl}cyclopentyl [(4-nitrophenyl)oxy]methanoate (120 mg, 0.224 mmol, 43.7%) as a yellow solid. LCMS: ESI m/z 536 [M+H]⁺.

Step D: (1R,3S)-3-{5-[(3-methyl-2-oxobenzo[d][1,3]oxazol-6-yl)amino]-2H-pyrazol-3-yl}cyclopentyl [(4-nitrophenyl)oxy]methanoate A mixture of (1R,3S)-3-{5-[(3-methyl-2-oxobenzo[d][1,3]oxazol-6-yl)amino]-1-(2-methylprop-2-yl)pyrazol-3-yl}cyclopentyl [(4-nitrophenyl)oxy]methanoate (120 mg, 0.224 mmol) in HCOOH (5 mL) was stirred at 100° C. overnight. The cooled reaction mixture concentrated under reduced pressure to give crude (1R,3S)-3-{5-[(3-methyl-2-oxobenzo[d][1,3]oxazol-6-yl)amino]-2H-pyrazol-3-yl}cyclopentyl [(4-nitrophenyl)oxy]methanoate (80 mg, 0.167 mmol, 74.5%) as a brown oil. LCMS: m/z 480 [M+H]⁺.

Step E: (1R,3S)-3-{5-[(3-methyl-2-oxobenzo[d][1,3]oxazol-6-yl)amino]-2H-pyrazol-3-yl}cyclopentyl (prop-2-ylamino)methanoate To a solution of (1R,3S)-3-{5-[(3-methyl-2-oxobenzo[d][1,3]oxazol-6-yl)amino]-2H-pyrazol-3-yl}cyclopentyl [(4-nitrophenyl)oxy]methanoate (80 mg, 0.167 mmol) in THF (2 mL) was added propan-2-amine (0.043 mL, 0.501 mmol) and DIEA EthyldiisopropylaMine (0.083 mL, 0.501 mmol) at rt. After stirred at rt for 30 min, the reaction mixture was concentrated. The residue was purified by prep-HPLC to give (1R,3S)-3-{5-[(3-methyl-2-oxobenzo[d][1,3]oxazol-6-yl)amino]-2H-pyrazol-3-yl}cyclopentyl (prop-2-ylamino)methanoate (10.5 mg, 0.026 mmol, 15.8%) as a white solid. LCMS: ESI m/z 400 [M+H]⁺. ¹H NMR (400 MHz, DMSO) δ 7.42 (s, 1H), 7.01-6.94 (m, 2H), 6.85 (d, J=7.6 Hz, 1H), 5.57 (s, 1H), 4.91 (s, 1H), 3.21 (s, 3H), 3.02-2.92 (m, 1H), 2.45-2.35 (m, 1H), 1.97-1.88 (m, 1H), 1.86-1.76 (m, 1H), 1.72-1.58 (m, 2H), 1.56-1.46 (m, 1H), 1.01-0.89 (m, 6H).

### Example 35: (1R,3S)-3-{5-[(1,1-dioxo-2,3-dihydro-1λ6-benzo[2,1-b]thiophen-7-yl)amino]-2H-pyrazol-3-yl}cyclopentyl (prop-2-ylamino)methanoate

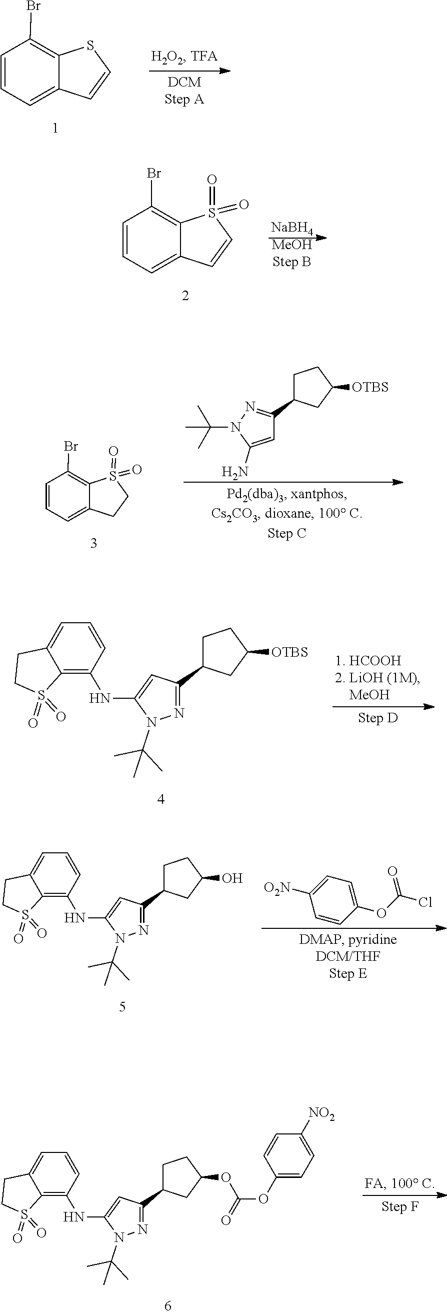

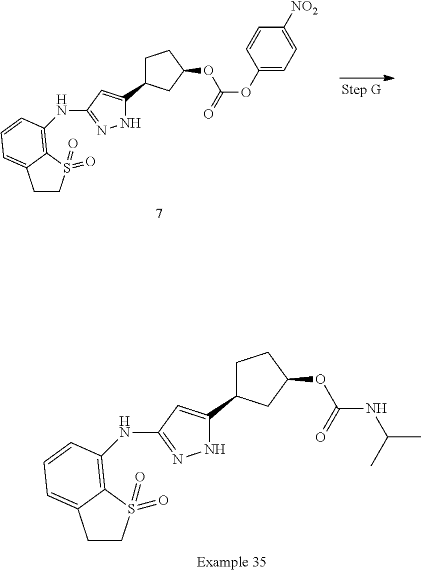 [SMILES: O=C(Oc1ccc([N+](=O)[O-])cc1)O[C@@H]1CC[C@H](c2cc(Nc3cccc4c3S(=O)(=O)CC4)n[nH]2)C1 ; CC(C)NC(=O)O[C@@H]1CC[C@H](c2cc(Nc3cccc4c3S(=O)(=O)CC4)n[nH]2)C1]

Step A: 7-bromo-1λ⁶-benzothiophene-1,1-dione To a solution of 7-bromobenzothiophene (2 g, 9.38 mmol) in DCM (20 mL) was added H₂O₂(4 mL, 39.2 mmol, 30% in H₂O) and 2,2,2-trifluoroacetic acid (4 mL, 53.8 mmol). The reaction mixture was stirred at 20° C. for 5 hours. Ice water was added to the reaction mixture, and then Na₂S₂O₃ (aqueous) was added. The following mixture was extracted with ethyl acetate, washed with sodium carbonate aqueous solution twice, washed with brine, dried over anhydrous sodium sulfate and concentrated. The residue was purified by flash chromatography eluted with ethyl acetate in petroleum ether (0˜35% gradient) to 7-bromo-1λ⁶-benzothiophene-1,1-dione (1.8 g, 7.34 mmol, 78.2%) as a white solid. LCMS: ESI m/z 245 [M+H]⁺.

Step B: 7-bromo-2,3-dihydro-1)⁶-benzothiophene-1,1-dione To a solution of 7-bromo-1λ⁶-benzothiophene-1,1-dione (1.8 g, 7.34 mmol) in MeOH (40 mL) was added NaBH₄ (555 mg, 14.6 mmol) at 0° C. The reaction mixture was stirred at 25° C. for 2 hours. After 2 h, the reaction mixture was quenched with water (2 mL) and then concentrated under reduced pressure. The residue was diluted with water and extracted with DCM. The organic phase was washed with brine, dried over anhydrous Na₂SO₄, filtered and concentrated in vacuo to afford 7-bromo-2,3-dihydro-1λ⁶-benzothiophene-1,1-dione (1.4 g, 5.66 mmol, 77.1%) as a white solid, which was used in next step without further purification. LCMS: ESI m/z 247 [M+H]⁺.

Step C: 7-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1λ⁶-benzothiophene-1,1-dione To a solution of 7-bromo-2,3-dihydro-1λ⁶-benzothiophene-1,1-dione (300 mg, 1.214 mmol) in dioxane (10 mL) were added 5-[(1S,3R)-3-{[dimethyl(2-methylprop-2-yl)silyl]oxy}cyclopentyl]-2-(2-methylprop-2-yl)pyrazol-3-amine (327 mg, 0.971 mmol), Cs₂CO₃ (791.11 mg, 2.428 mmol), Xant-PHOS (140 mg, 0.243 mmol) and Pd₂(dba)₃ (111 mg, 0.121 mmol). The reaction mixture was degassed with N₂ and stirred at 100° C. under N₂ atmosphere overnight. The cooled reaction mixture was diluted with EA and water. The organic layer was separated, washed with further brine, and concentrated in vacuo. The residue was purified using silica gel column chromatography eluted with ethyl acetate in petroleum ether (0-50% gradient) to afford the title compound 7-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1λ⁶-benzothiophene-1,1-dione (400 mg, 0.794 mmol, 65.4%) as a brown oil. LCMS: ESI m/z 504 [M+H]⁺.

Step D: 7-({5-[(1S,3R)-3-hydroxycyclopentyl]-2-(2-methylprop-2-yl)pyrazol-3-yl}amino)-2,3-dihydro-1λ⁶-benzothiophene-1,1-dione A mixture of 7-({5-[(1S,3R)-3-{[dimethyl(2-methylprop-2-yl)silyl]oxy}cyclopentyl]-2-(2-methylprop-2-yl)pyrazol-3-yl}amino)-2,3-dihydro-1⁶-benzothiophene-1,1-dione (400 mg, 0.794 mmol) in formic acid (6 mL) was stirred at RT for 1 h and then concentrated under reduced pressure. The residue was treated with a mixture of lithium hydroxlithium hydroxide hydrate (66.6 mg, 1.58 mmol) in MeOH (6 mL)/H₂O (1 mL). The resulting mixture was stirred at rt for 1 h, the mixture was concentrated under reduced pressure. The mixture was poured into water and extracted with EtOAc. The combined organic phase was washed with brine, dried with Na₂SO₄, filtered and concentrated. The residue was purified by chromatography eluted with ethyl acetate in petroleum ether (0-55% gradient) to give 7-({5-[(1S,3R)-3-hydroxycyclopentyl]-2-(2-methylprop-2-yl)pyrazol-3-yl}amino)-2,3-dihydro-1λ⁶-benzothiophene-1,1-dione (280 mg, 0.719 mmol, 90.5%) as a colorless oil. LCMS: ESI m/z 390 [M+H]⁺.

Step E: (1R,3S)-3-{5-[(1,1-dioxo-2,3-dihydro-1λ⁶-benzothiophen-7-yl)amino]-1-(2-methylprop-2-yl)pyrazol-3-yl}cyclopentyl [(4-nitrophenyl)oxy]methanoate To a solution of 7-({5-[(1S,3R)-3-hydroxycyclopentyl]-2-(2-methylprop-2-yl)pyrazol-3-yl}amino)-2,3-dihydro-1λ⁶-benzothiophene-1,1-dione (280 mg, 0.719 mmol) in DCM (5 mL) and THF (5 mL) were added 4-nitrophenyl chloromethanoate (434 mg, 2.16 mmol), Py (0.174 mL, 2.16 mmol) and DMAP (8.78 mg, 0.072 mmol). The reaction mixture was stirred under at 25° C. for 2 hr. The reaction solution was diluted with DCM, washed with water and brine, dried over anhydrous Na₂SO₄, concentrated in vacuum and the residue was purified by flash chromatography to afford (1R,3S)-3-{5-[(1,1-dioxo-2,3-dihydro-1λ⁶-benzothiophen-7-yl)amino]-1-(2-methylprop-2-yl)pyrazol-3-yl}cyclopentyl [(4-nitrophenyl)oxy]methanoate (330 mg, 0.595 mmol, 82.7%) as a colorless oil. LCMS: ESI m/z 555 [M+H]⁺.

Step F: (1R,3S)-3-{5-[(1,1-dioxo-2,3-dihydro-1λ⁶-benzo[2,1-b]thiophen-7-yl)amino]-2H-pyrazol-3-yl}cyclopentyl [(4-nitrophenyl)oxy]methanoate A solution of (1R,3S)-3-{5-[(1,1-dioxo-2,3-dihydro-1λ⁶-benzothiophen-7-yl)amino]-1-(2-methylprop-2-yl)pyrazol-3-yl}cyclopentyl [(4-nitrophenyl)oxy]methanoate (100 mg, 0.180 mmol) in formic acid (5 mL) was stirred at 100° C. overnight. The cooled reaction mixture was concentrated to afford (1R,3S)-3-{5-[(1,1-dioxo-2,3-dihydro-1λ⁶-benzo[2,1-b]thiophen-7-yl)amino]-2H-pyrazol-3-yl}cyclopentyl [(4-nitrophenyl)oxy]methanoate (100 mg, 0.160 mmol, 89.0%) as a yellow oil. LCMS: ESI m/z 499 [M+H]⁺.

Step G: (1R,3S)-3-{5-[(1,1-dioxo-2,3-dihydro-1λ⁶-benzo[2,1-b]thiophen-7-yl)amino]-2H-pyrazol-3-yl}cyclopentyl (prop-2-ylamino)methanoate A solution of (1R,3S)-3-{5-[(1,1-dioxo-2,3-dihydro-1λ⁶-benzo[2,1-b]thiophen-7-yl)amino]-2H-pyrazol-3-yl}cyclopentyl [(4-nitrophenyl)oxy]methanoate (100 mg, 0.160 mmol) in propan-2-amine (3 mL, 35.0 mmol) was stirred at rt for 1 h. The reaction concentrated. The residue was purified by prep-HPLC to afford the title compound (1R,3S)-3-{5-[(1,1-dioxo-2,3-dihydro-1λ⁶-benzo[2,1-b]thiophen-7-yl)amino]-2H-pyrazol-3-yl}cyclopentyl (prop-2-ylamino)methanoate (23 mg, 0.055 mmol, 34.2%) as a white solid. LCMS: ESI m/z 419 [M+H]⁺. ¹H NMR (400 MHz, DMSO) δ 12.08 (s, 1H), 7.69 (s, 1H), 7.44 (t, J=8.0 Hz, 1H), 7.21 (s, 1H), 6.96 (d, J=7.6 Hz, 1H), 6.82 (d, J=7.6 Hz, 1H), 5.94 (s, 1H), 5.00 (brs, 1H), 3.65-3.51 (m, 3H), 3.27 (t, J=6.8 Hz, 2H), 3.09-3.01 (m, 1H), 2.52-2.43 (m, 1H), 2.07-1.98 (m, 1H), 1.95-1.84 (m, 1H), 1.77-1.63 (m, 2H), 1.62-1.54 (m, 1H), 1.06-0.99 (m, 6H).

### Example 36: (1R,3S)-3-(3-((1,1-dioxido-2,3-dihydrobenzo[b]thiophen-4-yl)amino)-1H-pyrazol-5-yl)cyclopentyl isopropylcarbamate

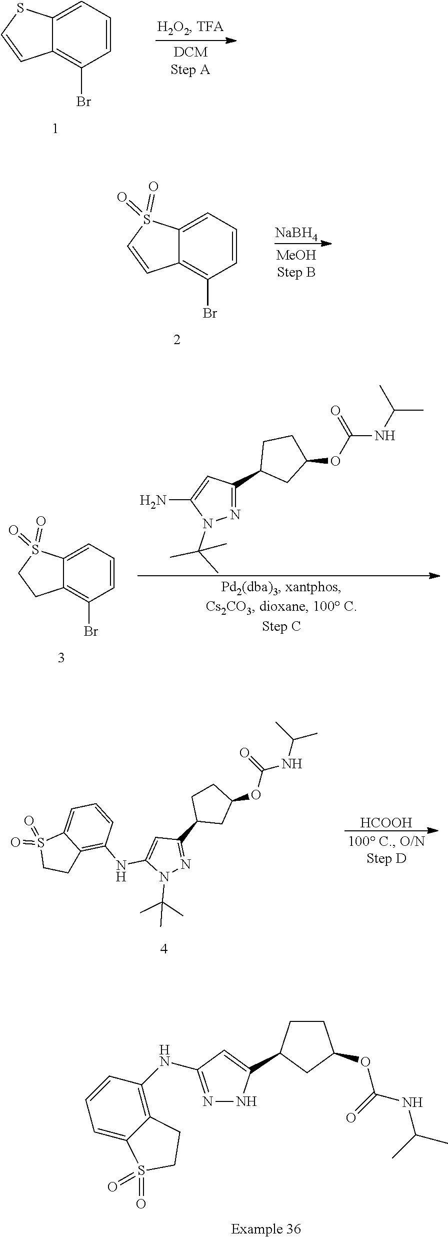

Step A: 4-bromobenzo[b]thiophene 1,1-dioxide To a stirred solution of 4-bromobenzo[b]thiophene (2.0 g, 9.4 mmol) in DCM (16 mL) were added H₂O₂(4 mL, 39.2 mmol, 30% in H₂O) and TFA (4 mL, 52.3 mmol) at room temperature. After stirring at room temperature for 5 h, the mixture was poured into Na₂S₂O₃(aq) and extracted with DCM. The combined organic phase was washed with brine, dried over Na₂SO₄, filtered and concentrated. The residue was purified by chromatography eluting with EA in PE [Gradient: 10%] to give 4-bromobenzo[b]thiophene 1,1-dioxide (1.7 g, 7.0 mmol, 74%) as a white solid. ¹H NMR (400 MHz, DMSO) δ 7.92 (dd, J=10.8, 7.6 Hz, 2H), 7.67-7.49 (m, 3H).

Step B: 4-bromo-2,3-dihydrobenzo[b]thiophene 1,1-dioxide To a solution of 4-bromobenzo[b]thiophene 1,1-dioxide (1.7 g, 7.0 mmol) in MeOH (18 mL) was added NaBH₄ (0.453 g, 11.97 mmol) at 0° C. The reaction mixture was stirred at room temperature for 2 h. The reaction was completed by detection of LCMS. The residue was concentrated and dissolved in EA, washed with H₂O and brine, dried over Na₂SO₄, filtered, concentrated, and purified by silica gel chromatography eluting with EA in PE [Gradient:10%] to afford 4-bromo-2,3-dihydrobenzo[b]thiophene 1,1-dioxide (1.70 g, 6.91 mmol, 98.7%) as a white solid. LCMS: ESI m/z 247 [M+H]⁺.

Step C: (1R,3S)-3-{5-[(1,1-dioxo-2,3-dihydro-1λ\hat{ }6-benzothiophen-4-yl)amino]-1-(2-methylprop-2-yl)pyrazol-3-yl}cyclopentyl (prop-2-ylamino)methanoate To a solution of 4-bromo-2,3-dihydrobenzo[b]thiophene 1,1-dioxide (52.0 mg, 0.211 mmol) in dioxane (5 mL) were added Cs₂CO₃ (105 mg, 0.322 mmol), (1R,3S)-3-[5-amino-1-(2-methylprop-2-yl)pyrazol-3-yl]cyclopentyl (prop-2-ylamino)methanoate (50 mg, 0.162 mmol), Pd₂(dba)₃ (14.8 mg, 0.016 mmol) and Xant-PHOS (18.7 mg, 0.032 mmol). The reaction mixture was stirred at100° C. under N₂ overnight. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 70%] to afford the title compound (1R,3S)-3-{5-[(1,1-dioxo-2,3-dihydro-16-benzothiophen-4-yl)amino]-1-(2-methylprop-2-yl)pyrazol-3-yl}cyclopentyl (prop-2-ylamino)methanoate (30 mg, 0.063 mmol, 39%) as a white solid. LCMS: ESI m/z 475 [M+H]⁺.

Step D: (1R,3S)-3-(3-((1,1-dioxido-2,3-dihydrobenzo[b]thiophen-4-yl)amino)-1H-pyrazol-5-yl)cyclopentyl isopropylcarbamate A solution of (1R,3S)-3-{5-[(1,1-dioxo-2,3-dihydro-1λ6-benzothiophen-4-yl)amino]-1-(2-methylprop-2-yl)pyrazol-3-yl}cyclopentyl (prop-2-ylamino)methanoate (30 mg, 0.063 mmol) in HCOOH (3 mL) and was stirred 100° C. overnight. The cooled reaction mixture was concentrated. The residue was purified by prep-HPLC to afford the title compound (1R,3S)-3-(3-((1,1-dioxido-2,3-dihydrobenzo[b]thiophen-4-yl)amino)-1H-pyrazol-5-yl)cyclopentyl isopropylcarbamate (4.2 mg, 0.01 mmol, 16%) as a white solid. LCMS: ESI m/z 419 [M+H]⁺. ¹H NMR (400 MHz, MeOD) δ 7.61 (brs, 1H), 7.36 (t, J=8.0 Hz, 1H), 7.09 (d, J=8.0 Hz, 1H), 5.88 (s, 1H), 5.09 (brs, 1H), 3.75-3.65 (m, 1H), 3.57-3.46 (m, 2H), 3.28-3.22 (m, 2H), 3.20-3.11 (m, 1H), 2.59-2.49 (m, 1H), 2.21-2.11 (m, 1H), 1.99-1.70 (m, 4H), 1.10 (t, J=6.0 Hz, 6H).

### Example 37: (1s,4s)-4-(3-((1,1-dioxido-2,3-dihydrobenzo[d]isothiazol-6-yl)amino)-1H-pyrazol-5-yl)cyclohexyl isopropylcarbamate

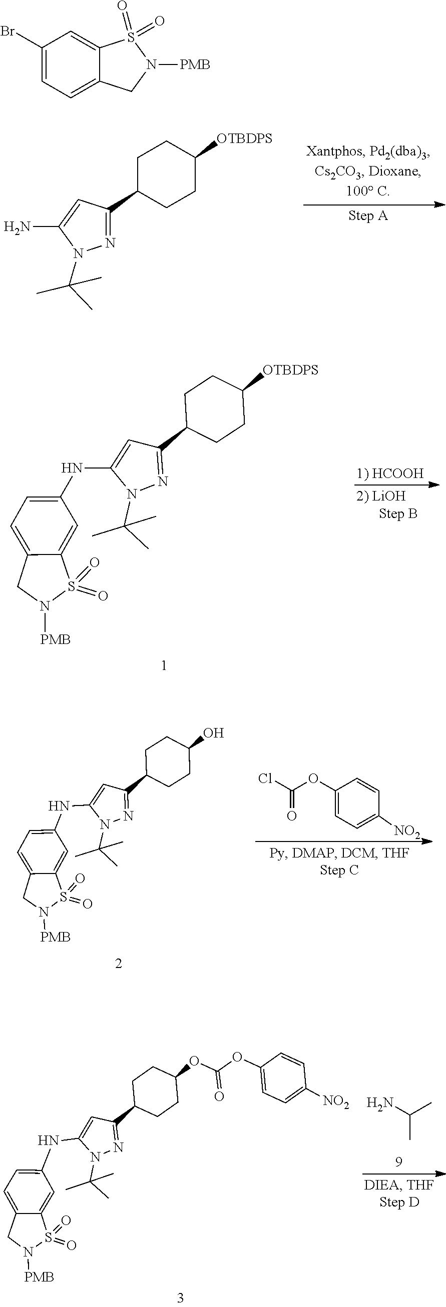

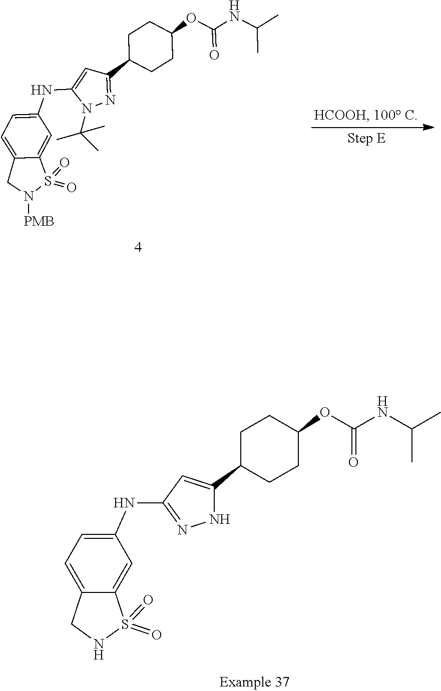

Step A: 6-((1-(tert-butyl)-3-((1s,4s)-4-((tert-butyldiphenylsilyl)oxy)cyclohexyl)-1H-pyrazol-5-yl)amino)-2-(4-methoxybenzyl)-2,3-dihydrobenzo[d]isothiazole 1,1-dioxide To a solution of 1-(tert-butyl)-3-((1s,4s)-4-((tert-butyldiphenylsilyl)oxy)cyclohexyl)-1H-pyrazol-5-amine (400 mg, 0.841 mmol) in dioxane (20 mL) was added 6-bromo-2-(4-methoxybenzyl)-2,3-dihydrobenzo[d]isothiazole 1,1-dioxide (400 mg, 1.09 mmol), Cs₂CO₃ (1.06 g, 3.26 mmol), Pd₂(dba)₃ (100 mg, 0.109 mmol) and Xantphos (126 mg, 0.217 mmol). The reaction mixture was stirred at 100° C. under N₂ for 1 hr. The reaction was complete detected by LCMS. The cooled reaction mixture was dissolved in EA (50 mL), washed with H₂O (20 mL) and brine, dried over sodium sulfate and concentrated. The residue was purified by silica gel chromatography (eluting with 20-100% EA in PE) to afford 6-((1-(tert-butyl)-3-((1s,4s)-4-((tert-butyldiphenylsilyl)oxy)cyclohexyl)-1H-pyrazol-5-yl)amino)-2-(4-methoxybenzyl)-2,3-dihydrobenzo[d]isothiazole 1,1-dioxide (500 mg, 0.655 mmol, 60.3%) as a yellow solid. LCMS: ESI m/z 763.09 [M+H]⁺.

Step B: 6-((1-(tert-butyl)-3-((1s,4s)-4-hydroxycyclohexyl)-1H-pyrazol-5-yl)amino)-2-(4-methoxybenzyl)-2,3-dihydrobenzo[d]isothiazole 1,1-dioxide A solution of 6-((1-(tert-butyl)-3-((1s,4s)-4-((tert-butyldiphenylsilyl)oxy)cyclohexyl)-1H-pyrazol-5-yl)amino)-2-(4-methoxybenzyl)-2,3-dihydrobenzo[d]isothiazole 1,1-dioxide (500 mg, 0.655 mmol) in formic acid (6 mL) was stirred at room temperature overnight. The reaction was complete detected by LCMS. The solvents were removed under vacuum. Then add EtOH (3 mL), H₂O (3 mL) and Lithium Hydroxide Monohydrate (3.30 mg, 0.0790 mmol) to alkaline pH. The reaction mixture was stirred at room temperature for 1 hr. LCMS showed the reaction was completed. The reaction mixture was dissolved in EA (50 mL), washed with H₂O (10 mL) and brine, dried over sodium sulfate and concentrated. The residue was purified by silica gel chromatography (eluting with 0-12% MeOH in DCM) to afford 6-((1-(tert-butyl)-3-((1s,4s)-4-hydroxycyclohexyl)-1H-pyrazol-5-yl)amino)-2-(4-methoxybenzyl)-2,3-dihydrobenzo[d]isothiazole 1,1-dioxide (100 mg, 0.248 mmol, 53.0%) as a white solid. LCMS: ESI m/z 525.68 [M+H]⁺.

Step C: (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl (4-nitrophenyl) carbonate A suspension of 6-((1-(tert-butyl)-3-((1s,4s)-4-hydroxycyclohexyl)-1H-pyrazol-5-yl)amino)-2-(4-methoxybenzyl)-2,3-dihydrobenzo[d]isothiazole 1,1-dioxide (200 mg, 0.381 mmol), DMAP (4.66 mg, 0.0380 mmol), pyridine (0.0920 mL, 1.14 mmol) and 4-nitrophenyl chloromethanoate (384 mg, 1.91 mmol) in DCM (3 mL) and THF (3 mL) was stirred at room temperature overnight. The reaction was completed by LCMS. The solvents were removed under vacuum. The reaction mixture was dissolved in EA (50 mL), washed with H₂O (5 mL) and brine, dried over sodium sulfate and concentrated. The residue was purified by silica gel chromatography (eluting with 0-12% MeOH in DCM) to afford (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl (4-nitrophenyl) carbonate (140 mg, 0.203 mmol, 53.3%) as a white solid. LCMS: ESI m/z 690.78 [M+H]⁺.

Step D: (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl isopropylcarbamate To a suspension of (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl (4-nitrophenyl) carbonate (140 mg, 0.203 mmol) in THF (3 mL) was added propan-2-amine (0.0870 mL, 1.02 mmol) and DIEA (0.101 mL, 0.609 mmol). The reaction mixture was stirred at room temperature for 3 hrs. LCMS showed the reaction had been completed. The reaction mixture was dissolved in EA (50 mL), washed with H₂O (10 mL) and brine, dried over sodium sulfate and concentrated. The residue was purified by silica gel chromatography (eluting with 0-12% MeOH in DCM) to afford (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl isopropylcarbamate (100 mg, 0.164 mmol, 80.8%) as a yellow solid. LCMS: 610.79 [M+H]⁺.

Step E: (1s,4s)-4-(3-((1,1-dioxido-2,3-dihydrobenzo[d]isothiazol-6-yl)amino)-1H-pyrazol-5-yl)cyclohexyl isopropylcarbamate A solution of (1s,4s)-4-(1-(tert-butyl)-5-((2-(4-methoxybenzyl)-1,1-dioxido-2,3-dihydrobenzo[d]isothiazol-6-yl)amino)-1H-pyrazol-3-yl)cyclohexyl isopropylcarbamate (100 mg, 0.164 mmol) in formic acid (4 mL) was stirred at 100° C. overnight. LCMS showed the reaction was completed. The reaction mixture was concentrated and purified by pre-HPLC (C18, 30˜ 95% ACN in H₂O with 0.1% TFA) to afford (1s,4s)-4-(3-((1,1-dioxido-2,3-dihydrobenzo [d]isothiazol-6-yl)amino)-1H-pyrazol-5-yl)cyclohexyl isopropylcarbamate (11.0 mg, 0.0250 mmol, 15.5%) as a white solid. LCMS: ESI m/z 434.53 [M+H]⁺. ¹H NMR (400 MHz, DMSO) δ 8.87 (s, 1H), 7.98 (s, 1H), 7.63 (brs, 1H), 7.38 (dd, J=8.4, 2.0 Hz, 1H), 7.31 (d, J=8.4 Hz, 1H), 6.89 (d, J=6.0 Hz, 1H), 5.61 (s, 1H), 4.75 (s, 1H), 4.26 (s, 2H), 2.72-2.66 (m, 1H), 1.85-1.63 (m, 8H), 1.05 (d, J=6.4 Hz, 6H).

### Example: 38 Synthesis of (1R,3S)-3-(3-((2,2-dioxido-1,3-dihydrobenzo[c]isothiazol-6-yl)amino)-1H-pyrazol-5-yl)cyclopentyl isopropylcarbamate

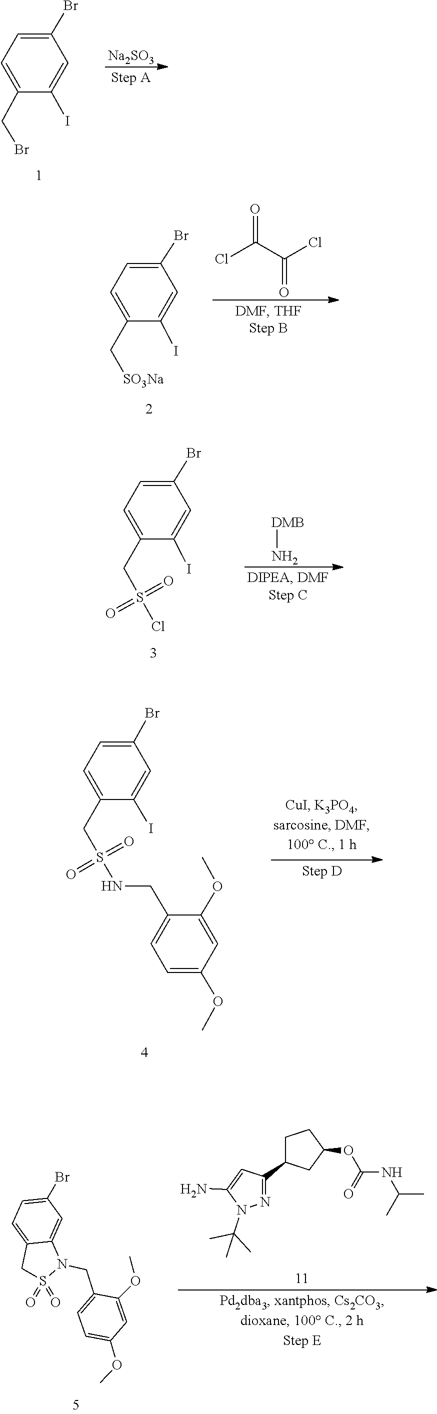

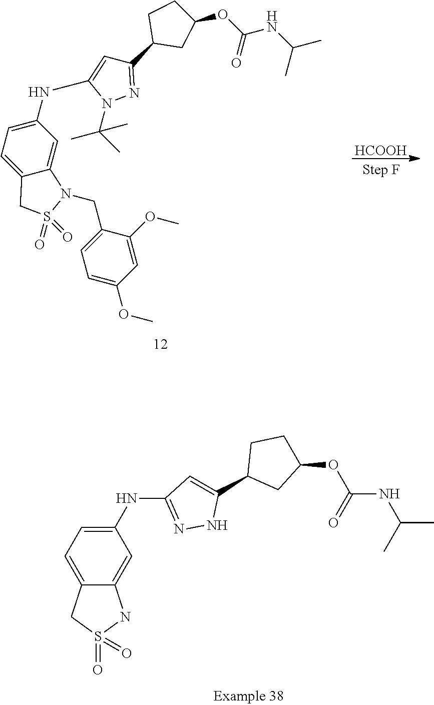 [SMILES: COc1ccc(CN2c3cc(Nc4cc([C@H]5CC[C@@H](OC(=O)NC(C)C)C5)nn4C(C)(C)C)ccc3CS2(=O)=O)c(OC)c1 ; "HCOOH" ; CC(C)NC(=O)O[C@@H]1CC[C@H](c2cc(Nc3ccc4c(c3)NS(=O)(=O)C4)n[nH]2)C1]

Step A. (4-bromo-2-iodophenyl)methanesulfonic acid. To a stirred solution of 4-bromo-1-(bromomethyl)-2-iodobenzene (5 g, 13 mmol) in actone (60 mL)/H₂O (40 mL) was added sodium sulfite (2 g, 16 mmol) at rt. After stirred at 90° C. overnight, the cooled mixture was concentrated to give crude (4-bromo-2-iodophenyl)methanesulfonic acid (5.5 g, 11.7 mmol, 88%) as a white solid. LCMS: m/z 375 [M+H]⁻.

Step B. (4-bromo-2-iodophenyl)methanesulfonyl chloride. To a stirred solution of sodium (4-bromo-2-iodophenyl)methanesulfonate (2.1 g, 5.3 mmol) in THF (40 mL) was added DMF (1 mL) and 2-chloro-2-oxoacetyl chloride (4.5 mL, 52 mmol) slowly at −20° C. After stirred at −20° C. for 2 h, the reaction mixture was poured into ice-water (20 mL) and extracted with EtOAc (10 mL*2). The combined organic phase was washed by brine, dried over Na₂SO₄, filtered and concentrated to give crude (4-bromo-2-iodophenyl)methanesulfonyl chloride (1.5 g, 3.8 mmol, 72%) as a yellow solid. LCMS: m/z 494 [M+H]⁻.

Step C. 1-(4-bromo-2-iodophenyl)-N-[(2,4-dimethoxyphenyl)methyl]methanesulfonamide. To a stirred solution of (4-bromo-2-iodophenyl)methanesulfonyl chloride (1.5 g, 3.8 mmol) in DMF (20 mL) was added (2,4-dimethoxyphenyl)methanamine (2.8 mL, 19 mmol) and DIPEA (3.3 mL, 19 mmol) slowly at rt. After stirred at rt for 1 h, the reaction mixture was poured into water (30 mL) and extracted with EtOAc (20 mL*2). The combined organic phase was washed with brine, dried with Na₂SO₄, filtered and concentrated. The residue was purified by chromatography (silica gel, 0-30%, EtOAc in PE) to give 1-(4-bromo-2-iodophenyl)-N-[(2,4-dimethoxyphenyl)methyl]methanesulfonamide (1.2 g, 2.3 mmol, 60%) as a yellow solid. LCMS: m/z 524 [M+H]⁻.

Step D. 6-bromo-1-[(2,4-dimethoxyphenyl)methyl]-1,3-dihydro-2λ<sup>6</sup>-benzo[2,1-c][1,2]thiazole-2,2-dione. To a stirred solution of 1-(4-bromo-2-iodophenyl)-N-[(2,4-dimethoxyphenyl)methyl]methanesulfonamide (650 mg, 1.2 mmol) in DMF (3 mL) was added CuI (47 mg, 0.25 mmol), Sarcosine (22 mg, 0.25 mmol) and K₃PO₄ (1.3 g, 6.2 mmol) at rt. After stirred at 100° C. for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-40%, EtOAc in PE) to give 6-bromo-1-[(2,4-dimethoxyphenyl)methyl]-1,3-dihydro-2λ⁶-benzo[2,1-c][1,2]thiazole-2,2-dione (420 mg, 1.1 mmol, 85%) as a yellow solid. LCMS: m/z 398 [M+H]⁺.

Step E. (1R,3S)-3-(1-(tert-butyl)-5-((1-(2,4-dimethoxybenzyl)-2,2-dioxido-1,3-dihydrobenzo[c]isothiazol-6-yl)amino)-1H-pyrazol-3-yl)cyclopentyl isopropylcarbamate. To a stirred solution of 6-bromo-1-[(2,4-dimethoxyphenyl)methyl]-1,3-dihydro-2⁶-benzo[2,1-c][1,2]thiazole-2,2-dione (90 mg, 0.23 mmol) in dioxane (6 mL) was added (1R,3S)-3-(5-amino-1-(tert-butyl)-1H-pyrazol-3-yl)cyclopentyl isopropylcarbamate (69 mg, 0.23 mmol), Xant-PHOS (26 mg, 0.045 mmol), Pd₂(dba)₃ (20 mg, 0.023 mmol) and Cs₂CO₃ (221 mg, 0.68 mmol) at rt. After stirred at 100° C. under N₂ for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-60%, EtOAc in PE) to give (1R,3S)-3-[5-({1-[(2,4-dimethoxyphenyl)methyl]-2,2-dioxo-1,3-dihydro-2λ⁶-benzo[2,1-c][1,2]thiazol-6-yl}amino)-2-(2-methylprop-2-yl)pyrazol-3-yl]cyclopentyl (prop-2-ylamino)methanoate (130 mg, 0.21 mmol, 92%) as a brown solid. LCMS: m/z 626 [M+H]⁺.

Step F. (1R,3S)-3-(3-((2,2-dioxido-1,3-dihydrobenzo[c]isothiazol-6-yl)amino)-1H-pyrazol-5-yl)cyclopentyl isopropylcarbamate A mixture of (1R,3S)-3-[5-({1-[(2,4-dimethoxyphenyl)methyl]-2,2-dioxo-1,3-dihydro-2λ⁶-benzo[2,1-c][1,2]thiazol-6-yl}amino)-2-(2-methylprop-2-yl)pyrazol-3-yl]cyclopentyl (prop-2-ylamino)methanoate (120 mg, 0.19 mmol) in HCOOH (1 mL) was stirred at 100° C. overnight. The cooled mixture was concentrated. The residue was purified by chromatography (silica gel, 0-10%, MeOH in DCM) and then prep-HPLC (C18, 40˜ 90% MeCN in H₂O with 0.1% NH₃·H₂O) to give (1R,3S)-3-(3-((2,2-dioxido-1,3-dihydrobenzo[c]isothiazol-6-yl)amino)-1H-pyrazol-5-yl)cyclopentyl isopropylcarbamate (5 mg, 0.012 mmol, 6%) as a white solid. LCMS: m/z 420 [M+H]⁺. ¹H NMR (400 MHz, DMSO) δ 10.25 (s, 1H), 8.45 (s, 1H), 7.06-6.99 (m, 2H), 6.97-6.91 (m, 1H), 6.78 (dd, J=8.4, 1.6 Hz, 1H), 5.62 (s, 1H), 5.00 (s, 1H), 4.35 (s, 2H), 3.61-3.57 (m, 1H), 3.07-3.01 (m, 1H), 2.53-2.47 (m, 1H), 2.05-1.98 (m, 1H), 1.97-1.89 (m, 1H), 1.76-1.58 (m, 3H), 1.03 (d, J=6.4 Hz, 6H).

### Example 39: Synthesis of (1R,3S)-3-(3-((1,1-dioxidothiochroman-7-yl)amino)-1H-pyrazol-5-yl)cyclopentyl isopropylcarbamate

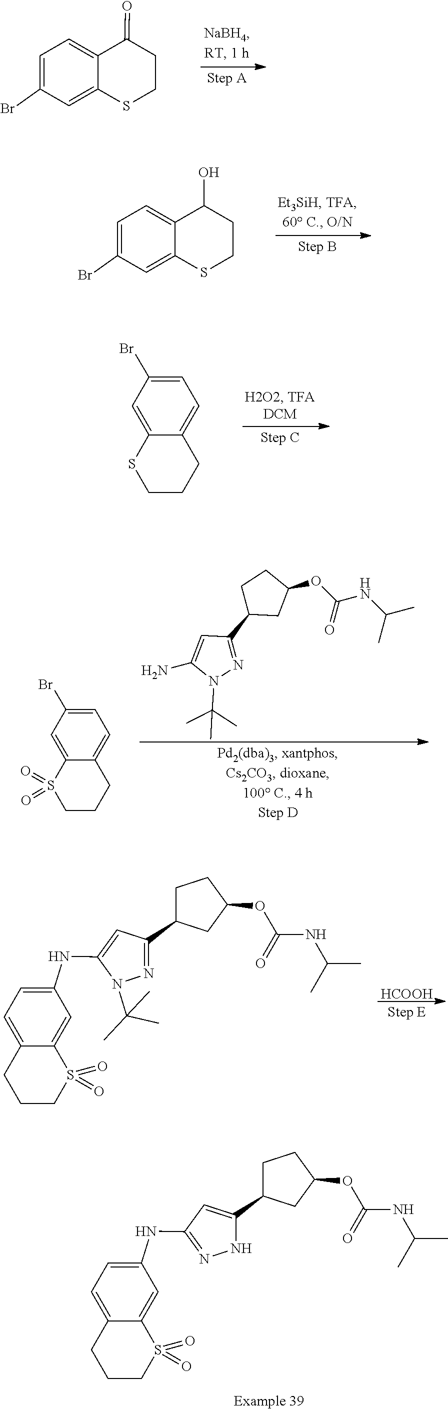

Step A: 7-bromo-3,4-dihydro-2H-1-benzothiin-4-ol To a solution of 7-bromothiochroman-4-one (900 mg, 3.702 mmol) in MeOH (10 mL) was added NaBH₄ (0.242 mL, 7.404 mmol), and the reaction was stirred at room temperature for 3 hr. The reaction was diluted with DCM and water. The organic layer was separated, washed with further brine, and concentrated in vacuo to afford crude the title compound 7-bromo-3,4-dihydro-2H-1-benzothiin-4-ol (900 mg, 3.672 mmol, 99.2%) as a transparent oil. ¹H NMR (400 MHz, DMSO) δ 7.28 (dd, J=6.4, 5.2 Hz, 2H), 7.21 (dd, J=8.2, 2.0 Hz, 1H), 5.44 (d, J=5.2 Hz, 1H), 4.62-4.54 (m, 1H), 3.20-3.10 (m, 1H), 3.00-2.92 (m, 1H), 2.10-2.03 (m, 1H), 2.00-1.90 (m, 1H).

Step B: 7-bromo-3,4-dihydro-2H-1-benzothiine To a solution of 7-bromo-3,4-dihydro-2H-1-benzothiin-4-ol (900 mg, 3.672 mmol) in TFA (10 mL) was added triethylsilane (3.56 mL, 22.0 mmol), and the reaction was stirred at 60° C. for 18 hr. Adjust the pH of reaction solution to 5 with 2M sodium hydroxide aqueous solution. Then the reaction was diluted with DCM (100 mL) and water (100 mL). The organic layer was separated, washed with further brine, and concentrated in vacuo. The residue was purified using silica gel column chromatography eluting with 100% Hexane to afford the title compound 7-bromo-3,4-dihydro-2H-1-benzothiine (700 mg, 3.06 mmol, 83.21%) as a transparent oil. ¹H NMR (400 MHz, CDCl3) δ 7.23 (s, 1H), 7.07 (dd, J=8.0, 2.0 Hz, 1H), 6.86 (d, J=8.0 Hz, 1H), 3.03-2.97 (m, 2H), 2.77-2.70 (m, 2H), 2.11-2.02 (m, 2H)

Step C: 7-bromothiochromane 1,1-dioxide To a solution of 7-bromo-3,4-dihydro-2H-1-benzothiine (70 mg, 0.31 mmol) in DCM (5 mL) were added TFA (0.045 mL, 0.61 mmol) and H₂O₂(0.1 ml, 0.98 mmol, 30% solution), and the reaction was stirred at room temperature for 18 hr. The reaction was diluted with DCM and saturated Na₂S₂O₃ solution. The organic layer was separated, washed with further brine, and concentrated in vacuo. The residue was purified using silica gel column chromatography eluting with ethyl acetate in petroleum ether (0%-10%) to afford the title compound 7-bromothiochromane 1,1-dioxide (50 mg, 0.191 mmol, 62.7%) as a white solid. LCMS:263.1 [M+H]⁺

Step D: (1R,3S)-3-(1-(tert-butyl)-5-((1,1-dioxidothiochroman-7-yl)amino)-1H-pyrazol-3-yl)cyclopentyl isopropylcarbamate To a solution of 7-bromothiochromane 1,1-dioxide (50 mg, 0.191 mmol) in dioxane (10 mL) was added (1R,3S)-3-[5-amino-1-(2-methylprop-2-yl)pyrazol-3-yl]cyclopentyl (prop-2-ylamino)methanoate (70.87 mg, 0.230 mmol), Xant-PHOS (22.16 mg, 0.038 mmol) and Cs₂CO₃ (124.77 mg, 0.383 mmol). The reaction mixture was bubbled through with N₂ for 5 minutes. Pd₂(dba)₃ (17.5 mg, 0.019 mmol) was added and the reaction stirred at 100° C. under N₂ for 18 hr. The cooled reaction mixture was quenched by adding sat. aq. NH₄Cl solution, extracted with EA. The organic phases was washed with brine, dried over anhydrous Na₂SO₄, filtered and concentrated in vacuo. The residue was purified by column chromatography to give (1R,3S)-3-(1-(tert-butyl)-5-((1,1-dioxidothiochroman-7-yl)amino)-1H-pyrazol-3-yl)cyclopentyl isopropylcarbamate (50 mg, 0.102 mmol, 53.44%) as a yellow oil. LCMS:489.4 [M+H]⁺

Step E: (1R,3S)-3-(3-((1,1-dioxidothiochroman-7-yl)amino)-1H-pyrazol-5-yl)cyclopentyl isopropylcarbamate A solution of (1R,3S)-3-(1-(tert-butyl)-5-((1,1-dioxidothiochroman-7-yl)amino)-1H-pyrazol-3-yl)cyclopentyl isopropylcarbamate (80 mg, 0.164 mmol) in HCOOH (10 mL) was stirred at 100° C. for 18 hr. The cooled reaction mixture was concentrated. The residue was purified by prep-HPLC (C18, 5%-95% MeCN in H₂O with 0.1% HCOOH) to give (1R,3S)-3-(3-((1,1-dioxidothiochroman-7-yl)amino)-1H-pyrazol-5-yl)cyclopentyl isopropylcarbamate (25 mg, 0.058 mmol, 35.30%) as a white solid. LCMS: 433 [M+H]⁺. H NMR (400 MHz, DMSO) δ 11.82 (s, 1H), 8.66 (s, 1H), 8.03 (d, J=2.4 Hz, 1H), 7.32 (dd, J=8.4, 2.4 Hz, 1H), 7.12 (d, J=8.4 Hz, 1H), 6.95 (brd, J=7.6 Hz, 1H), 5.58 (d, J=2.0 Hz, 1H), 5.02-4.97 (m, 1H), 3.66-3.47 (m, 1H), 3.44-3.37 (m, 2H), 3.10-2.98 (m, 1H), 2.88-2.81 (m, 2H), 2.47-2.40 (m, 1H), 2.30-2.22 (m, 2H), 2.06-1.96 (m, 1H), 1.95-1.83 (m, 1H), 1.77-1.66 (m, 2H), 1.65-1.52 (m, 1H), 1.03 (d, J=6.4 Hz, 6H).

### Example 40: (1R,3S)-3-{5-[(2,2-dioxo-1,3-dihydro-2λ⁶-benzo[c]thiophen-4-yl)amino]-2H-pyrazol-3-yl}cyclopentyl (prop-2-ylamino)methanoate

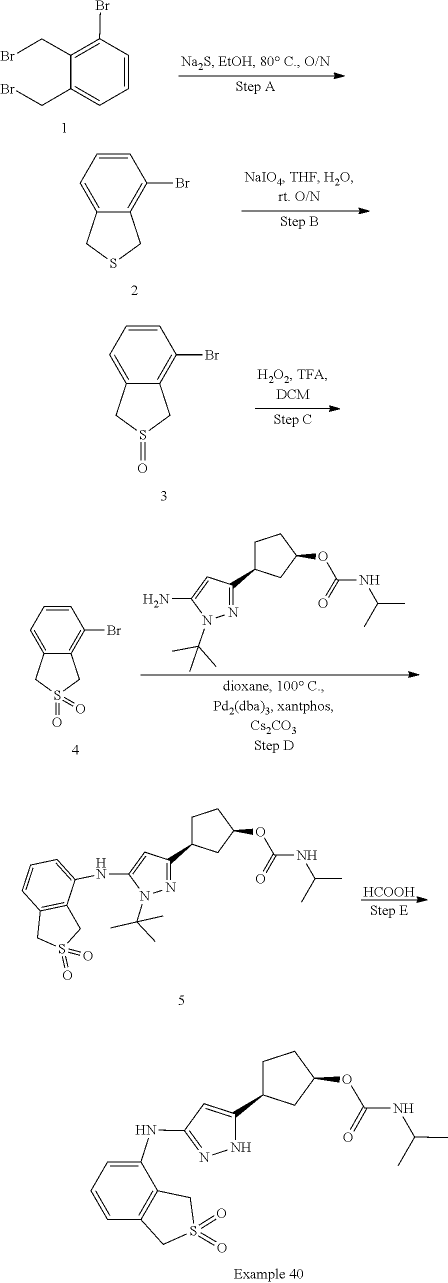

Step A: 4-bromo-1,3-dihydrobenzo[c]thiophene To a solution of 1-bromo-2,3-bis(bromomethyl)benzene (2 g, 5.83 mmol) in EtOH (5 mL) and H₂O (1 mL) was added Na₂S (0.50 g, 6.42 mmol). The reaction was stirred at 80° C. overnight. The cooled reaction mixture was diluted with EA and water. The organic layer was separated, washed with brine, dried over Na₂SO₄ and concentrated in vacuo. The residue was purified using silica gel column chromatography eluted with ethyl acetate in petroleum ether (gradient: 0-5%) to afford the title compound 4-bromo-1,3-dihydrobenzo[c]thiophene (550 mg, 2.55 mmol, 43.8%) as a yellow oil.

Step B: 4-bromo-1,3-dihydro-2)⁴-benzo[c]thiophen-2-one To a solution of 4-bromo-1,3-dihydrobenzo[c]thiophene (250 mg, 1.162 mmol) in THF (12 mL) and H₂O (8.4 mL) was added sodium periodate (522 mg, 2.44 mmol) at 25° C. The reaction mixture was stirred at 25° C. overnight. TLC showed starting material was consumed and new spot was observed. The organic phases were washed with brine, dried over anhydrous Na₂SO₄ and concentrated in vacuo. The residue was purified by column chromatography eluted with ethyl acetate in petroleum ether (0˜95% gradient) to give 4-bromo-1,3-dihydro-2?⁴-benzo[c]thiophen-2-one (220 mg, 0.952 mmol, 81.9%) as a yellow solid. LCMS: ESI m/z 231 [M+H]⁺.

Step C: 4-bromo-1,3-dihydro-2)⁶-benzo[c]thiophene-2,2-dione To a solution of 4-bromo-1,3-dihydro-2?⁴-benzo[c]thiophen-2-one (120 mg, 0.519 mmol) in DCM (3 mL) was added Hydrogen peroxide (1 mL, 9.8 mmol, 30% in H₂O) and 2,2,2-trifluoroacetic acid (1 mL, 13.4 mmol). The reaction mixture was stirred at 20° C. for 3 hours. Ice water was added to the reaction mixture, and then Na₂S₂O₃ (aqueous) was added. The following mixture was extracted with ethyl acetate. The organic phase was washed sodium carbonate aqueous solution and brine in turn, dried over Na₂SO₄ and concentrated. The residue was purified by flash chromatography eluted with ethyl acetate in petroleum ether (0-60% gradient) to 4-bromo-1,3-dihydro-2λ⁶-benzo[c]thiophene-2,2-dione (45 mg, 0.182 mmol, 35.0%) as a white solid. LCMS: ESI m/z 247 [M+H]⁺.

Step D: (1R,3S)-3-{5-[(2,2-dioxo-1,3-dihydro-2λ⁶-benzo[c]thiophen-4-yl)amino]-1-(2-methylprop-2-yl)pyrazol-3-yl}cyclopentyl (prop-2-ylamino)methanoate To a solution of 4-bromo-1,3-dihydro-2λ⁶-benzo[c]thiophene-2,2-dione (65 mg, 0.263 mmol) in dioxane (5 mL) were added (1R,3S)-3-[5-amino-1-(2-methylprop-2-yl)pyrazol-3-yl]cyclopentyl (prop-2-ylamino)methanoate (81.1 mg, 0.263 mmol), Cs₂CO₃ (171.41 mg, 0.526 mmol), Xant-PHOS (30.4 mg, 0.053 mmol) and Pd₂(dba)₃ (24.1 mg, 0.026 mmol). The reaction mixture was degassed with N₂ and stirred at 100° C. under N₂ atmosphere overnight. The cooled reaction mixture was diluted with EA and water. The organic layer was separated, washed with brine, dried over Na₂SO₄ and concentrated. The residue was purified using silica gel column chromatography eluted with ethyl acetate in petroleum ether (0-40% gradient) to afford (1R,3S)-3-{5-[(2,2-dioxo-1,3-dihydro-2λ⁶-benzo[c]thiophen-4-yl)amino]-1-(2-methylprop-2-yl)pyrazol-3-yl}cyclopentyl (prop-2-ylamino)methanoate (100 mg, 0.211 mmol, 80.10%) as a brown oil. LCMS: ESI m/z 475 [M+H]⁺.

Step E: (1R,3S)-3-{5-[(2,2-dioxo-1,3-dihydro-2λ⁶-benzo[c]thiophen-4-yl)amino]-2H-pyrazol-3-yl}cyclopentyl (prop-2-ylamino)methanoate A solution of (1R,3S)-3-{5-[(2,2-dioxo-1,3-dihydro-2λ⁶-benzo[c]thiophen-4-yl)amino]-1-(2-methylprop-2-yl)pyrazol-3-yl}cyclopentyl (prop-2-ylamino)methanoate (100 mg, 0.211 mmol) in formic acid (5 mL) was stirred at 100° C. overnight. The cooled reaction mixture was concentrated. The residue was purified by prep-HPLC to afford the title compound (1R,3S)-3-{5-[(2,2-dioxo-1,3-dihydro-2λ⁶-benzo[c]thiophen-4-yl)amino]-2H-pyrazol-3-yl}cyclopentyl (prop-2-ylamino)methanoate (35 mg, 0.084 mmol, 39.69%) as a white solid. LCMS: ESI m/z 419 [M+H]⁺. ¹H NMR (400 MHz, DMSO) δ 11.87 (s, 1H), 7.67 (s, 1H), 7.60 (d, J=7.6 Hz, 1H), 7.19 (t, J=8.0 Hz, 1H), 6.94-6.89 (m, 1H), 6.74 (d, J=7.6 Hz, 1H), 5.73 (s, 1H), 5.00 (brs, 1H), 4.45 (s, 2H), 4.35 (s, 2H), 3.58-3.52 (m, 1H), 3.12-2.99 (m, 1H), 2.51-42 (m, 1H), 2.08-1.97 (m, 1H), 1.96-1.84 (m, 1H), 1.75-1.67 (m, 2H), 1.66-1.58 (m, 1H), 1.03 (d, J=6.4 Hz, 6H).

### Example 41: Synthesis of (1R,3S)-3-{5-[(1-methyl-2,2-dioxo-1,3-dihydro-2X<sup>6</sup>-benzo[2,1-c][1,2]thiazol-6-yl)amino]-2H-pyrazol-3-yl}cyclopentyl (prop-2-ylamino)methanoate

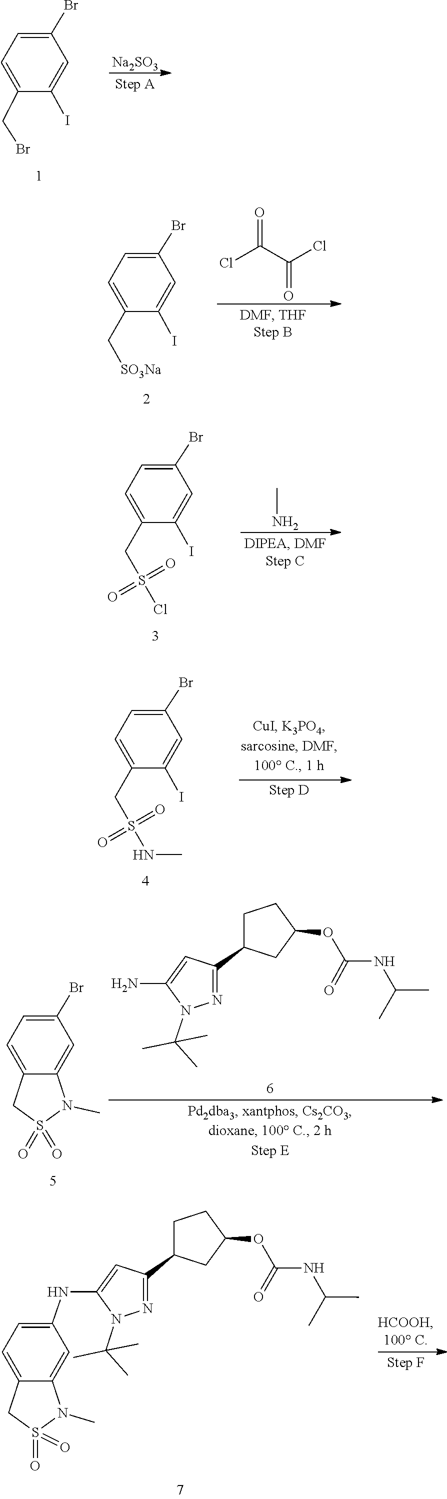

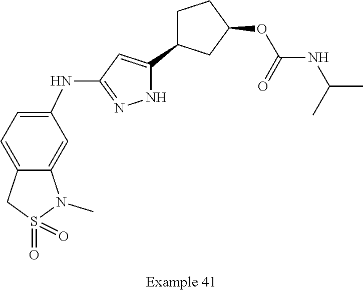 [SMILES: CC(C)NC(=O)O[C@@H]1CC[C@H](c2cc(Nc3ccc4c(c3)N(C)S(=O)(=O)C4)n[nH]2)C1]

Step A. Sodium (4-bromo-2-iodophenyl)methanesulfonate. To a stirred mixture of 4-bromo-1-(bromomethyl)-2-iodobenzene (3 g, 8 mmol) in actone (60 mL)/H₂O (40 mL) was added sodium sulfite (1.2 g, 9.579 mmol) at rt. After stirred at 90° C. for 3 h, the cooled mixture was concentrated to give crude sodium (4-bromo-2-iodophenyl)methanesulfonate (5.5 g, 11 mmol, 88%) as a white solid. LCMS: m/z 375 [M+H]⁻.

Step B. (4-bromo-2-iodophenyl)methanesulfonyl chloride. To a stirred mixture of sodium (4-bromo-2-iodophenyl)methanesulfonate (500 mg, 1.3 mmol) in THF (20 mL) was added DMF (0.1 mL) and 2-chloro-2-oxoacetyl chloride (7.6 mL, 88 mmol) slowly at −20° C. After stirred at −20° C. for 2 h, the mixture was poured into ice-water (20 mL) and extracted with EtOAc (10 mL*2). The combined organic phase was washed by brine, dried over Na₂SO₄, filtered and concentrated to give crude (4-bromo-2-iodophenyl)methanesulfonyl chloride (2.5 g, 6.3 mmol, 72%) as a yellow solid. LCMS: m/z 494 [M+H]⁻.

Step C. 1-(4-bromo-2-iodophenyl)-N-methylmethanesulfonamide. To a stirred solution of (4-bromo-2-iodophenyl)methanesulfonyl chloride (1.5 g, 3.8 mmol) in DMF (20 mL) was added methanamine (3.8 mL, 7.6 mmol, 2M in THF) and DIPEA (3.3 mL, 18.9 mmol) slowly at rt. After stirred at rt for 1 h, the mixture was poured into water (30 mL) and extracted with EtOAc (20 mL*2). The combined organic phase was washed with brine, dried with Na₂SO₄, filtered and concentrated. The residue was purified by chromatography (silica gel, 0-30%, EtOAc in PE) to give 1-(4-bromo-2-iodophenyl)-N-methylmethanesulfonamide (1.2 g, 2.3 mmol, 60%) as a yellow solid. LCMS: m/z 388 [M+H]⁻.

Step D. 6-bromo-1-methyl-1,3-dihydro-2λ<sup>6</sup>-benzo[2,1-c][1,2]thiazole-2,2-dione. To a stirred solution of 1-(4-bromo-2-iodophenyl)-N-methylmethanesulfonamide (550 mg, 1.41 mmol) in DMF (6 mL) was added CuI (54 mg, 0.28 mmol), Sarcosine (25 mg, 0.28 mmol) and K₃PO₄ (1.5 g, 7.1 mmol) at rt. After stirred at 100° C. for 1 h, the cooled mixture was poured into water 10 (mL) and extracted with EtOAc (10 mL*2). The combined organic phase was washed with brine, dried with Na₂SO₄, filtered and concentrated. The residue was purified by chromatography (silica gel, 0-40%, EtOAc in PE) to give 6-bromo-1-methyl-1,3-dihydro-2λ⁶-benzo[2,1-c][1,2]thiazole-2,2-dione (350 mg, 1.3 mmol, 94%) as a yellow solid. No LCMS

Step E. (1R,3S)-3-{5-[(1-methyl-2,2-dioxo-1,3-dihydro-2λ<sup>6</sup>-benzo[2,1-c][1,2]thiazol-6-yl)amino]-2-(2-methylprop-2-yl)pyrazol-3-yl}cyclopentyl (prop-2-ylamino)methanoate. To a stirred solution of 6-bromo-1-methyl-1,3-dihydro-2λ⁶-benzo[2,1-c][1,2]thiazole-2,2-dione (102 mg, 0.39 mmol) in dioxane (6 mL) was added (1R,3S)-3-[5-amino-2-(2-methylprop-2-yl)pyrazol-3-yl]cyclopentyl (prop-2-ylamino)methanoate (120 mg, 0.39 mmol), Xant-PHOS (45 mg, 0.08 mmol), Pd₂(dba)₃ (35 mg, 0.04 mmol) and Cs₂CO₃ (380 mg, 1.2 mmol) at rt. After stirred at 100° C. for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-60%, EtOAc in PE) to give (1R,3S)-3-{5-[(1-methyl-2,2-dioxo-1,3-dihydro-2λ⁶-benzo[2,1-c][1,2]thiazol-6-yl)amino]-2-(2-methylprop-2-yl)pyrazol-3-yl}cyclopentyl (prop-2-ylamino)methanoate (150 mg, 0.3 mmol, 78%) as a brown solid. LCMS: m/z 490 [M+H]⁺.

Step F. (1R,3S)-3-{5-[(1-methyl-2,2-dioxo-1,3-dihydro-2%<sup>6</sup>-benzo[2,1-c][1,2]thiazol-6-yl)amino]-2H-pyrazol-3-yl}cyclopentyl (prop-2-ylamino)methanoate. A mixture of (1R,3S)-3-{5-[(1-methyl-2,2-dioxo-1,3-dihydro-2⁶-benzo[2,1-c][1,2]thiazol-6-yl)amino]-1-(2-methylprop-2-yl)pyrazol-3-yl}cyclopentyl (prop-2-ylamino)methanoate (100 mg, 0.2 mmol) in HCOOH (3 mL) was stirred at 100° C. overnight. The cooled reaction mixture was concentrated. The residue was purified by chromatography (silica gel, 0-10%, MeOH in DCM) and then prep-HPLC (C18, 40˜ 90% MeCN in H₂O with 0.1% HCOOH) to give (1R,3S)-3-{5-[(1-methyl-2,2-dioxo-1,3-dihydro-2λ⁶-benzo[2,1-c][1,2]thiazol-6-yl)amino]-2H-pyrazol-3-yl}cyclopentyl (prop-2-ylamino)methanoate (5 mg, 0.01 mmol, 5%) as a white solid. LCMS: m/z 434 [M+H]⁺. ¹H NMR (400 MHz, MeOD) δ 7.09 (d, J=8.0 Hz, 1H), 6.78 (s, 1H), 6.74 (dd, J=8.0, 2.0 Hz, 1H), 5.78 (s, 1H), 5.08 (s, 1H), 4.59 (s, 1H), 4.31 (s, 2H), 3.72-3.65 (m, 1H), 3.19-3.11 (m, 1H), 3.04 (s, 3H), 2.56-2.49 (m, 1H), 2.15-2.08 (m, 1H), 1.96-1.72 (m, 3H), 1.10 (d, J=6.0 Hz, 6H).

### Example 42 (1R,3S)-3-{5-[(1-methyl-2,2-dioxo-1,3-dihydro-2%6-benzo[c][1,2]thiazol-5-yl)amino]-2H-pyrazol-3-yl}cyclopentyl (prop-2-ylamino)methanoate

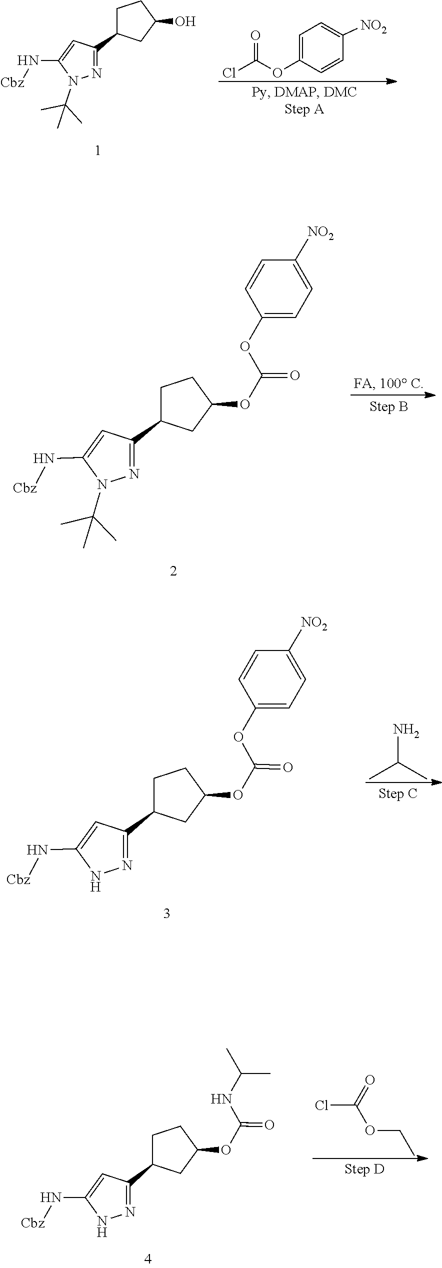

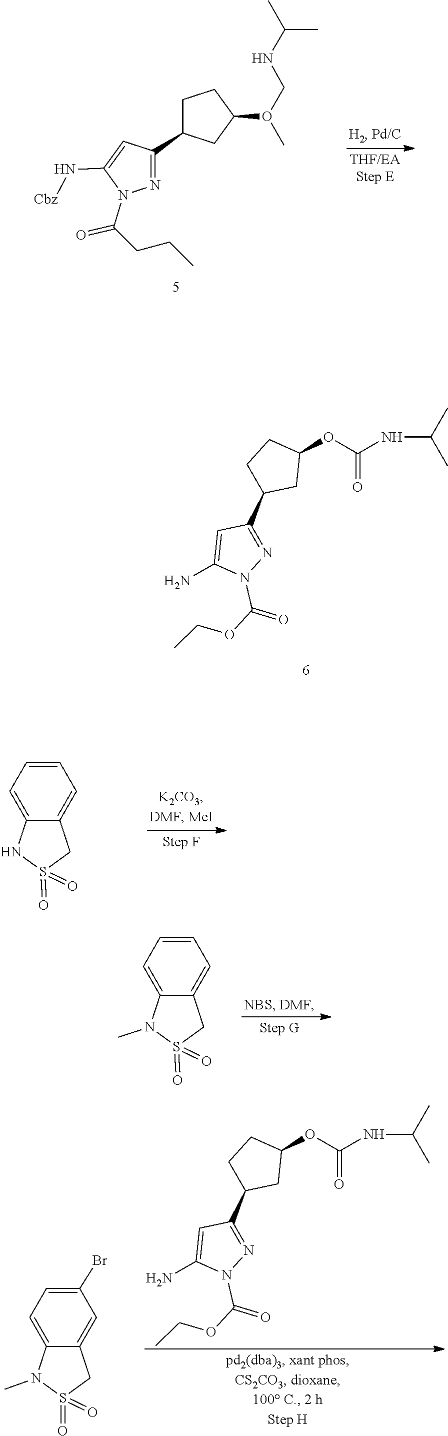

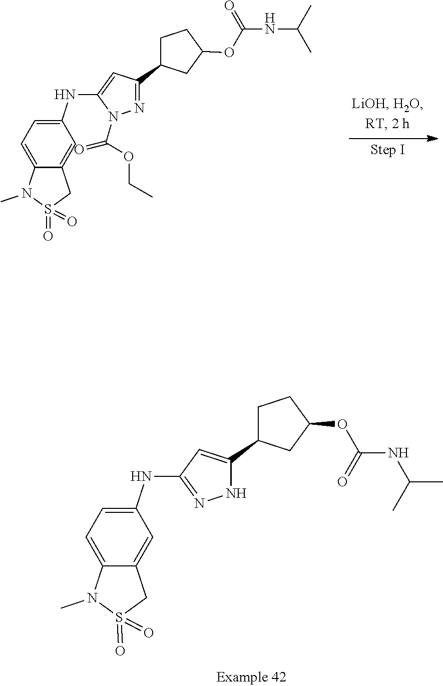

Step A: benzyl (1-(tert-butyl)-3-((1S,3R)-3-(((4-nitrophenoxy)carbonyl)oxy)cyclopentyl)-1H-pyrazol-5-yl)carbamate To a solution of benzyl (1-(tert-butyl)-3-((1S,3R)-3-hydroxycyclopentyl)-1H-pyrazol-5-yl)carbamate (500 mg, 1.4 mmol) in DCM (17 mL) were added 4-nitrophenyl chloroformate (422 mg, 2.1 mmol), DMAP (17 mg, 0.140 mmol), pyridine (0.34 mL, 4.2 mmol). The reaction was stirred at 25° C. for 3 hr. The reaction was concentrated in vacuo. The residue was purified using silica gel column chromatography eluting with EA in PE [Gradient: 0-100%] to afford benzyl (1-(tert-butyl)-3-((1S,3R)-3-(((4-nitrophenoxy)carbonyl)oxy)cyclopentyl)-1H-pyrazol-5-yl)carbamate (500 mg, 0.957 mmol, 68.4%) as a colorless oil. LCMS: ESI m/z 523 [M+H]⁺.

Step B: benzyl (3-((1S,3R)-3-(((4-nitrophenoxy)carbonyl)oxy)cyclopentyl)-1H-pyrazol-5-yl)carbamate A mixture of benzyl (1-(tert-butyl)-3-((1S,3R)-3-(((4-nitrophenoxy)carbonyl)oxy)cyclopentyl)-1H-pyrazol-5-yl)carbamate (500 mg, 0.957 mmol) in formic acid (5 mL) was stirred at 100° C. overnight. The cooled reaction mixture was concentrated. The residue was purified using silica gel column chromatography eluted with ethyl acetate in petroleum ether (gradient: 0-30%) to give benzyl (3-((1S,3R)-3-(((4-nitrophenoxy)carbonyl)oxy)cyclopentyl)-1H-pyrazol-5-yl)carbamate (200 mg, 0.43 mmol, 45%) as a yellow solid. LCMS: ESI m/z 467 [M+H]⁺.

Step C: benzyl (3-((1S,3R)-3-((isopropylcarbamoyl)oxy)cyclopentyl)-1H-pyrazol-5-yl)carbamate To a solution of benzyl (3-((1S,3R)-3-(((4-nitrophenoxy)carbonyl)oxy)cyclopentyl)-1H-pyrazol-5-yl)carbamate (200 mg, 0.43 mmol) in THF (2 mL) was added propan-2-amine (0.07 mL, 0.86 mmol), the reaction mixture was stirred at room temperature for 1 h. The reaction was concentrated. The residue was purified using silica gel column chromatography eluted with ethyl acetate in petroleum ether (gradient: 0-30%) to give benzyl (3-((1S,3R)-3-((isopropylcarbamoyl)oxy)cyclopentyl)-1H-pyrazol-5-yl)carbamate as a yellow solid. LCMS: ESI m/z 387 [M+H]⁺.

Step D: ethyl 5-(((benzyloxy)carbonyl)amino)-3-((1S,3R)-3-((isopropylcarbamoyl)oxy)cyclopentyl)-1H-pyrazole-1-carboxylate To a stirred solution of benzyl (3-((1S,3R)-3-((isopropylcarbamoyl)oxy)cyclopentyl)-1H-pyrazol-5-yl)carbamate (1.45 g, 3.75 mmol) and diisopropylethyl amine (1.45 g, 11.2 mmol) in dichloromethane (30 mL) was added dropwise ethyl chloroformate (970 mg, 8.94 mmol) at rt. The reaction mixture stirred at room temperature for 18 hours. The reaction was quenched with sat. aq NH₄Cl (3*5 mL), extracted with DCM. The organic phase was washed with brine, dried over Na₂SO₄, filtered, and concentrated to give crude ethyl 5-(((benzyloxy)carbonyl)amino)-3-((1S,3R)-3-((isopropylcarbamoyl)oxy)cyclopentyl)-1H-pyrazole-1-carboxylate (1.71 g, 100%) as a light yellow gum, which was used without further purification. LCMS: ESI m/z 459 [M+H]⁺.

Step E: ethyl 5-amino-3-((1S,3R)-3-((isopropylcarbamoyl)oxy)cyclopentyl)-1H-pyrazole-1-carboxylate To a solution of ethyl 5-(((benzyloxy)carbonyl)amino)-3-((1S,3R)-3-((isopropylcarbamoyl)oxy)cyclopentyl)-1H-pyrazole-1-carboxylate (1.71 g, 3.75 mmol) in THF (20 mL) and ethyl acetate (20 mL) was added Pd/C 10% (150 mg). The reaction mixture was stirred at RT under using a H₂ balloon for 4 h. LCMS showed the reaction was complete. The reaction mixture was filtered and the filtrate was concentrated in vacuo to afford ethyl 5-amino-3-((1S,3R)-3-((isopropylcarbamoyl)oxy)cyclopentyl)-1H-pyrazole-1-carboxylate (1.25 g, 100%) as a black solid. LCMS: ESI m/z 325 [M+H]⁺.

Step F: 1-methyl-1,3-dihydro-2λ6-benzo[c][1,2]thiazole-2,2-dione To a solution of 1,3-dihydro-2λ6-benzo[c][1,2]thiazole-2,2-dione (900 mg, 5.3 mmol) in DMF (5 mL) were added K₂CO₃ (735 mg, 5.3 mmol) and CH3I (3.02 g, 21.3 mmol). The reaction was stirred at room temperature overnight. The reaction was diluted with EtOAc and water. The organic layer was separated, washed with brine, dried over Na₂SO₄ and concentrated in vacuo. The residue was purified using silica gel column chromatography eluted with ethyl acetate in petroleum ether (gradient:0-30%) to afford the title compound 1-methyl-1,3-dihydro-2λ6-benzo[c][1,2]thiazole-2,2-dione (800 mg, 4.4 mmol, 82%) as a white solid. LCMS: m/z 183.22 [M+H]⁺.

Step G: 5-bromo-1-methyl-1,3-dihydro-2λ6-benzo[c][1,2]thiazole-2,2-dione To a solution of 1-methyl-1,3-dihydro-2λ6-benzo[c][1,2]thiazole-2,2-dione (800 mg, 4.4 mmol) in DMF (5 mL) was added NBS (777 mg, 4.4 mmol). The reaction mixture was stirred at room temperature overnight. The reaction mixture was diluted with EtOAc and water. The organic layer was separated, washed with brine and concentrated in vacuo. The residue was purified using silica gel column chromatography eluted with ethyl acetate in petroleum ether (gradient: 0-30%) to afford the title compound 5-bromo-1-methyl-1,3-dihydro-2λ6-benzo[c][1,2]thiazole-2,2-dione (1 g, 3.8 mmol, 87%) as a white solid. LCMS: m/z 397.48 [M+H]⁺.

Step H: ethyl 3-((1S,3R)-3-((isopropylcarbamoyl)oxy)cyclopentyl)-5-((1-methyl-2,2-dioxido-1,3-dihydrobenzo[c]isothiazol-5-yl)amino)-1H-pyrazole-1-carboxylate To a solution of 5-bromo-1-methyl-1,3-dihydro-2λ6-benzo[c][1,2]thiazole-2,2-dione (50 mg, 0.191 mmol) in dioxane (5 mL) were added ethyl 5-amino-3-[(1S,3R)-3-{[(prop-2-ylamino)carbonyl]oxy}cyclopentyl]pyrazole-1-carboxylate (47.6 mg, 0.147 mmol), Pd₂(dba)₃ (13.4 mg, 0.015 mmol), Xant-PHOS (17.0 mg, 0.029 mmol) and Cs₂CO₃ (143 mg, 0.440 mmol). The reaction was stirred at 100° C. for 2 h. The cooled reaction mixture was concentrated in vacuo to afford the title compound ethyl 3-((1S,3R)-3-((isopropylcarbamoyl)oxy)cyclopentyl)-5-((1-methyl-2,2-dioxido-1,3-dihydrobenzo[c]isothiazol-5-yl)amino)-1H-pyrazole-1-carboxylate (20 mg, 0.04 mmol, 31%) as a brown solid. LCMS: m/z 509.59 [M+H]⁺.

Step I: (1R,3S)-3-{5-[(1-methyl-2,2-dioxo-1,3-dihydro-2λ6-benzo[c][1,2]thiazol-5-yl)amino]-2H-pyrazol-3-yl}cyclopentyl (prop-2-ylamino)methanoate To a solution of ethyl 3-((1S,3R)-3-((isopropylcarbamoyl)oxy)cyclopentyl)-5-((1-methyl-2,2-dioxido-1,3-dihydrobenzo[c]isothiazol-5-yl)amino)-1H-pyrazole-1-carboxylate (50 mg, 0.1 mmol) in H₂O (5 mL) was added LiOH (4.15 mg, 0.099 mmol). The reaction mixture was stirred at room temperature for 2 h. The reaction mixture was diluted with EA and water. The organic layer was separated, washed with brine and concentrated in vacuo. The residue was purified using silica gel column chromatography eluted with ethyl acetate in petroleum ether (gradient: 0-50%) to afford the title compound (1R,3S)-3-{5-[(1-methyl-2,2-dioxo-1,3-dihydro-2λ6-benzo[c][1,2]thiazol-5-yl)amino]-2H-pyrazol-3-yl}cyclopentyl (prop-2-ylamino)methanoate (3 mg, 0.007 mmol, 7%) as a white solid. LCMS: m/z 433.53 [M+H]⁺. ¹H NMR (400 MHz, MeOD) δ 7.26 (s, 1H), 7.22 (d, J=8.4 Hz, 1H), 6.90 (d, J=8.4 Hz, 1H), 5.15-5.01 (m, 1H), 4.47 (s, 2H), 3.74-3.62 (m, 1H), 3.26-3.13 (m, 1H), 3.10 (s, 3H), 2.66-2.45 (m, 1H), 2.25-2.10 (m, 1H), 2.02-1.74 (m, 4H), 1.16-1.02 (m, 6H).

### Example 43 synthesis of (1R,3S)-3-{5-[(2,2-dioxo-1,3-dihydro-2%6-benzo[c]thiophen-5-yl)amino]-4-methyl-2H-pyrazol-3-yl}cyclopentyl (prop-2-ylamino)methanoate

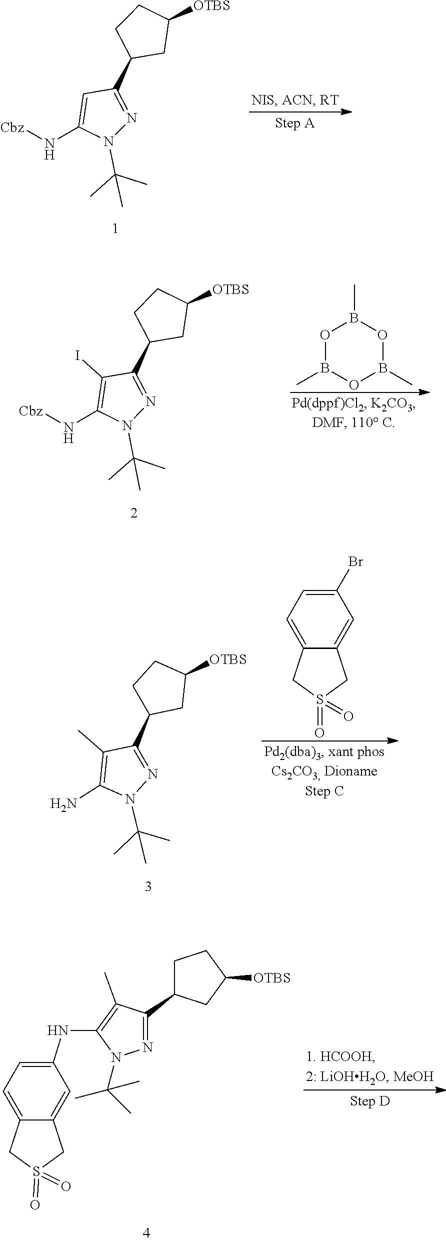

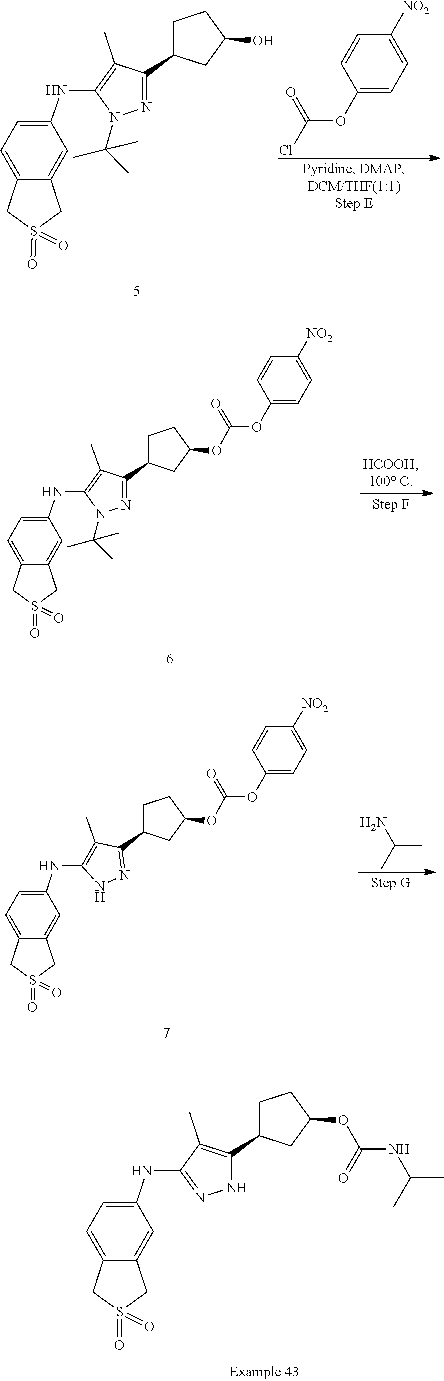

Step A: benzyl ({5-[(1S,3R)-3-{[dimethyl(2-methylprop-2-yl)silyl]oxy}cyclopentyl]-4-iodo-2-(2-methylprop-2-yl)pyrazol-3-yl}amino)methanoate To a solution of benzyl ({5-[(1S,3R)-3-{[dimethyl(2-methylprop-2-yl)silyl]oxy}cyclopentyl]-2-(2-methylprop-2-yl)pyrazol-3-yl}amino)methanoate (5 g, 10.6 mmol) in acetonitrile (10 mL) was added 1-iodotetrahydropyrrole-2,5-dione (2.62 g, 11.7 mmol). The reaction was stirred at room temperature for 3 hr. The reaction was diluted with DCM (100*2 mL) and water (200 mL). The organic layer was separated, washed with further brine (200 mL), dry with Na₂SO₄, and concentrated in vacuo. The residue was purified using silica gel column chromatography eluting with ethyl acetate in petroleum ether (1%-20%) to afford the title compound benzyl ({5-[(1S,3R)-3-{[dimethyl(2-methylprop-2-yl)silyl]oxy}cyclopentyl]-4-iodo-2-(2-methylprop-2-yl)pyrazol-3-yl}amino)methanoate (6.3 g, 10.5 mmol, 99.5%) as a yellow solid. LCMS: 598.1 [M+H]⁺

Step B: 5-[(1S,3R)-3-{[dimethyl(2-methylprop-2-yl)silyl]oxy}cyclopentyl]-4-methyl-2-(2-methylprop-2-yl)pyrazol-3-amine To a mixture of benzyl ({5-[(1S,3R)-3-{[dimethyl(2-methylprop-2-yl)silyl]oxy}cyclopentyl]-4-iodo-2-(2-methylprop-2-yl)pyrazol-3-yl}amino)methanoate (2.0 g, 3.35 mmol) and 2,4,6-trimethyl-1,3,5,2,4,6-trioxatriborinane (3.78 mL, 26.8 mmol) in DMF (20 mL) were added K₂CO₃ (1.39 g, 10.0 mmol) and Pd(dppf)Cl₂ (0.24 g, 0.335 mmol). The reaction was stirred at 110° C. under N₂ for 3 hr. The reaction was diluted with water and extracted with EA (50 mL*3). The combined organic layers were washed with brine, dried over Na₂SO₄, filtered and concentrated. The residue was purified using silica gel column chromatography eluting with PE/EA (100/1˜ 12/1) to afford the title compound 5-[(1S,3R)-3-{[dimethyl(2-methylprop-2-yl)silyl]oxy}cyclopentyl]-4-methyl-2-(2-methylprop-2-yl)pyrazol-3-amine (400 mg, 1.14 mmol, 34%) as a pink oil. LCMS: 352.4 [M+H]⁺.

Step C: 5-({5-[(1S,3R)-3-{[dimethyl(2-methylprop-2-yl)silyl]oxy}cyclopentyl]-4-methyl-2-(2-methylprop-2-yl)pyrazol-3-yl}amino)-1,3-dihydro-2λ6-benzo[c]thiophene-2,2-dione To a stirred solution of 5-[(1S,3R)-3-{[dimethyl(2-methylprop-2-yl)silyl]oxy}cyclopentyl]-4-methyl-2-(2-methylprop-2-yl)pyrazol-3-amine (200 mg, 0.569 mmol) in dioxane (10 mL) was added 5-bromo-1,3-dihydro-2λ6-benzo[c]thiophene-2,2-dione (155 mg, 0.626 mmol), Xant-PHOS (65.8 mg, 0.114 mmol) and Cs₂CO₃ (371 mg, 1.14 mmol). The reaction mixture was bubbled through with N₂ for 5 minutes. Pd₂(dba)₃ (52.09 mg, 0.057 mmol) was added, and the reaction mixture was stirred at 100° C. under N₂ for 3 hr. The cooled reaction mixture was diluted with water and extracted with EtOAc (20 mL*3). The combined organic layers were washed with brine, dried over Na₂SO₄, filtered and concentrated. The residue was purified using silica gel column chromatography eluting with PE/EA (100/1˜ 1/1) to afford the title compound 5-({5-[(1S,3R)-3-{[dimethyl(2-methylprop-2-yl)silyl]oxy}cyclopentyl]-4-methyl-2-(2-methylprop-2-yl)pyrazol-3-yl}amino)-1,3-dihydro-2λ6-benzo[c]thiophene-2,2-dione (80 mg, 0.154 mmol, 27%) as a yellow solid. LCMS: 516.9 [M−H]⁻

Step D: 5-({5-[(1S,3R)-3-hydroxycyclopentyl]-4-methyl-2-(2-methylprop-2-yl)pyrazol-3-yl}amino)-1,3-dihydro-2λ6-benzo[c]thiophene-2,2-dione A solution of 5-({5-[(1S,3R)-3-{[dimethyl(2-methylprop-2-yl)silyl]oxy}cyclopentyl]-4-methyl-2-(2-methylprop-2-yl)pyrazol-3-yl}amino)-1,3-dihydro-2λ6-benzo[c]thiophene-2,2-dione (80 mg, 0.154 mmol) in HCOOH (5 mL) was stirred at room temperature for 2 hr. The reaction was concentrated in vacuo. The residue was dissolved with methanol and adjusted to pH value 12-13 with 2 N lithium hydroxide aqueous solution. The reaction was diluted with EtOAc and water. The organic layer was separated, washed with brine and concentrated in vacuo. The residue was purified using silica gel column chromatography eluted with methanol in DCM (gradient:0-10%) to afford the title compound 5-({5-[(1S,3R)-3-hydroxycyclopentyl]-4-methyl-2-(2-methylprop-2-yl)pyrazol-3-yl}amino)-1,3-dihydro-2λ6-benzo[c]thiophene-2,2-dione (50 mg, 0.124 mmol, 80%) as a white solid. LCMS: 404.3 [M+H]⁺

Step E: (1R,3S)-3-{5-[(2,2-dioxo-1,3-dihydro-2λ6-benzo[c]thiophen-5-yl)amino]-4-methyl-1-(2-methylprop-2-yl)pyrazol-3-yl}cyclopentyl [(4-nitrophenyl)oxy]methanoate A suspension of 5-({5-[(1S,3R)-3-hydroxycyclopentyl]-4-methyl-2-(2-methylprop-2-yl)pyrazol-3-yl}amino)-1,3-dihydro-2λ6-benzo[c]thiophene-2,2-dione (50 mg, 0.124 mmol), DMAP (1.51 mg, 0.012 mmol), Pyridine (0.040 mL, 0.496 mmol) and 4-nitrophenyl chloromethanoate (49.95 mg, 0.248 mmol) in DCM (3.00 mL) and THF (3.00 mL) was stirred at 50° C. overnight. The solvents were removed under vacuum. The residue was dissolved in ethyl acetate (5 mL*2), washed with sat. aq NH₄Cl (10 mL) and brine, dried over sodium sulfate, filtered, concentrated, and purified by silica gel chromatography (eluting with 50% ethyl acetate in petroleum ether) to afford (1R,3S)-3-{5-[(2,2-dioxo-1,3-dihydro-2λ6-benzo[c]thiophen-5-yl)amino]-4-methyl-1-(2-methylprop-2-yl)pyrazol-3-yl}cyclopentyl [(4-nitrophenyl)oxy]methanoate (11 mg, 0.019 mmol, 16%) as a yellow solid. LCMS: 569.0 [M+H]⁺

Step F: (1R,3S)-3-{5-[(2,2-dioxo-1,3-dihydro-2λ6-benzo[c]thiophen-5-yl)amino]-4-methyl-1H-pyrazol-3-yl}cyclopentyl [(4-nitrophenyl)oxy]methanoate A solution of (1R,3S)-3-{5-[(2,2-dioxo-1,3-dihydro-2λ⁶-benzo[c]thiophen-5-yl)amino]-4-methyl-1-(2-methylprop-2-yl)pyrazol-3-yl}cyclopentyl [(4-nitrophenyl)oxy]methanoate (65 mg, 0.114 mmol) in HCOOH (10 mL) was stirred at 100° C. for 18 hr. The reaction was concentrated in vacuo to afford the title compound (1R,3S)-3-{5-[(2,2-dioxo-1,3-dihydro-2λ⁶-benzo[c]thiophen-5-yl)amino]-4-methyl-1H-pyrazol-3-yl}cyclopentyl [(4-nitrophenyl)oxy]methanoate (50 mg, 0.098 mmol, 85%) as a yellow solid. LCMS: 513.3 [M+H]⁺

Step G: (1R,3S)-3-{5-[(2,2-dioxo-1,3-dihydro-2λ⁶-benzo[c]thiophen-5-yl)amino]-4-methyl-2H-pyrazol-3-yl}cyclopentyl (prop-2-ylamino)methanoate A solution of (1R,3S)-3-{5-[(2,2-dioxo-1,3-dihydro-2λ⁶-benzo[c]thiophen-5-yl)amino]-4-methyl-1H-pyrazol-3-yl}cyclopentyl [(4-nitrophenyl)oxy]methanoate (10 mg, 0.020 mmol) in propan-2-amine (1.81 mL, 21.1 mmol) was stirred at room temperature for 2 hr. The reaction mixture was concentrated. The residue was purified by prep-HPLC (C18, 5%-95% MeCN in H₂O with 0.1% HCOOH) to give (1R,3S)-3-{5-[(2,2-dioxo-1,3-dihydro-2λ⁶-benzo[c]thiophen-5-yl)amino]-4-methyl-2H-pyrazol-3-yl}cyclopentyl (prop-2-ylamino)methanoate (2 mg, 0.005 mmol, 24%) as a white solid. LCMS: 433.2 [M+H]⁺. ¹H NMR (400 MHz, MeOD) δ 7.12 (d, J=8.4 Hz, 1H), 6.91 (d, J=7.6 Hz, 2H), 5.10 (s, 1H), 4.29 (d, J=6.8 Hz, 4H), 3.77-3.64 (m, 1H), 3.23-3.14 (m, 1H), 2.55-2.44 (m, 1H), 2.11-1.74 (m, 9H), 1.12 (dd, J=6.8, 2.4 Hz, 6H).

### Example 44: Synthesis of (1R,3S)-3-{5-[(2,2-dioxo-1,3-dihydro-2%6-benzo[c]thiophen-5-yl)amino]-4-methyl-2H-pyrazol-3-yl}cyclopentyl [(2-methylprop-2-yl)amino]methanoate

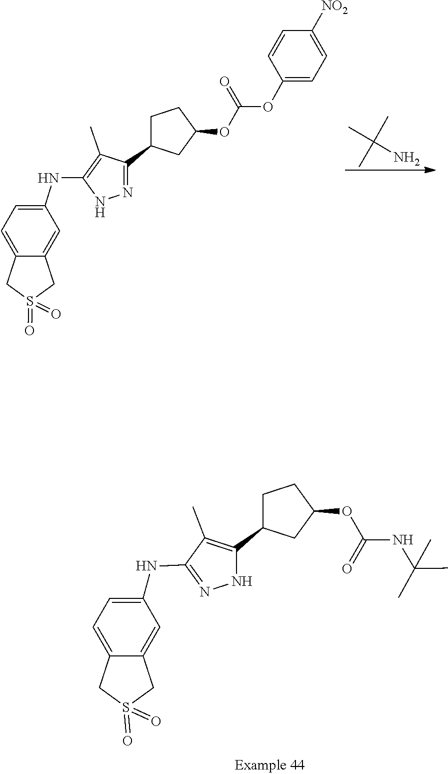

A solution of (1R,3S)-3-{5-[(2,2-dioxo-1,3-dihydro-2λ⁶-benzo[c]thiophen-5-yl)amino]-4-methyl-1H-pyrazol-3-yl}cyclopentyl [(4-nitrophenyl)oxy]methanoate (50 mg, 0.098 mmol) in 2-methylpropan-2-amine (5 mL, 47.2 mmol) was stirred at room temperature for 2 hr. The reaction mixture was concentrated. The residue was purified by prep-HPLC (C18, 5%-95% MeCN in H₂O with 0.1% HCOOH) to give (1R,3S)-3-{5-[(2,2-dioxo-1,3-dihydro-2⁶-benzo[c]thiophen-5-yl)amino]-4-methyl-2H-pyrazol-3-yl}cyclopentyl [(2-methylprop-2-yl)amino]methanoate (12 mg, 0.027 mmol, 28%) as a white solid. LCMS: 447.2 [M+H]⁺. ¹H NMR (400 MHz, DMSO) δ 7.87 (s, 1H), 7.26 (s, 1H), 7.13 (s, 2H), 6.79 (s, 1H), 4.97 (brs, 1H), 4.38 (s, 2H), 4.32 (s, 2H), 3.14-3.01 (m, 1H), 2.42-2.30 (m, 1H), 1.99-1.85 (m, 5H), 1.84-1.71 (m, 2H), 1.70-1.62 (sm, 1H), 1.22 (s, 9H).

### Example 45: (1R,3S)-3-{5-[(2,2-dioxo-1,3-dihydro-2λ6-benzo[c][1,2]thiazol-5-yl)amino]-2H-pyrazol-3-yl}cyclopentyl (prop-2-ylamino)methanoate

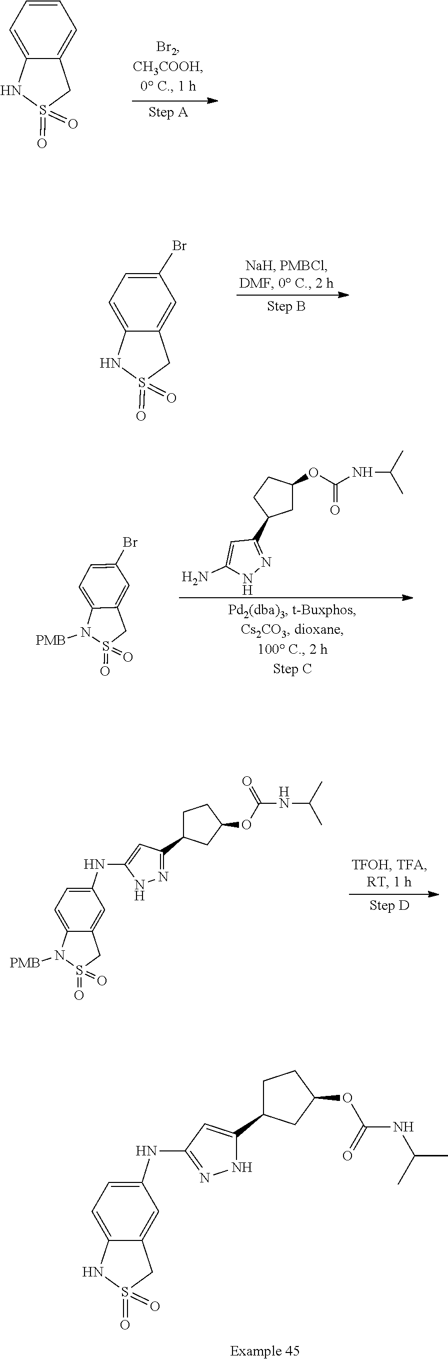

Step A: 5-bromo-1,3-dihydro-2λ6-benzo[c][1,2]thiazole-2,2-dione To a stirred solution of 1,3-dihydro-2λ6-benzo[c][1,2]thiazole-2,2-dione (1 g, 5.9 mmol) in acetic acid (10 mL) was added dropwise Br₂ (0.3 mL, 5.9 mmol) in 5 min. The reaction was stirred at 0° C. for 1 hr. TLC (PE/EA=5:1) showed starting material was consumed and new spot was observed. The reaction mixture was diluted with water, extracted with EA (50 mL×3). The organic layer was separated, washed with brine, washed with aqueous Na₂S₂O₃, dried over Na₂SO₄ and concentrated in vacuo. The residue was purified using silica gel column chromatography eluted with ethyl acetate in petroleum ether (gradient: 0-30%) to afford the title compound 5-bromo-1,3-dihydro-2λ6-benzo[c][1,2]thiazole-2,2-dione (1 g, 4.0 mmol, 68%) as a white solid. LCMS: m/z 248.09 [M+H]⁺.

Step B: 5-bromo-1-[(4-methoxyphenyl)methyl]-1,3-dihydro-26-benzo[c][1,2]thiazole-2,2-dione To a solution of 5-bromo-1,3-dihydro-2λ6-benzo[c][1,2]thiazole-2,2-dione (1 g, 4.0 mmol) in DMF (10 mL) and H₂O (0.2 mL) were added Cs₂CO₃ (2.63 g, 8.1 mmol) and 4-methoxybenzylchloride (0.95 g, 6.1 mmol). The reaction was stirred at 70° C. overnight. The cooled reaction mixture was diluted with EA and water. The organic layer was separated, washed with brine and concentrated in vacuo. The residue was purified using silica gel column chromatography eluted with ethyl acetate in petroleum ether (gradient: 0-20%) to afford the title compound 5-bromo-1-[(4-methoxyphenyl)methyl]-1,3-dihydro-2λ6-benzo[c][1,2]thiazole-2,2-dione (1.2 g, 3.3 mmol, 81%) as a yellow solid. LCMS: m/z 368.25 [M+H]⁺.

Step C: (1R,3S)-3-[5-({1-[(4-methoxyphenyl)methyl]-2,2-dioxo-1,3-dihydro-2λ6-benzo[c][1,2]thiazol-5-yl}amino)-1H-pyrazol-3-yl]cyclopentyl (prop-2-ylamino)methanoate. To a solution of 5-bromo-1-[(4-methoxyphenyl)methyl]-1,3-dihydro-2λ6-benzo[c][1,2]thiazole-2,2-dione (50 mg, 0.14 mmol) in dioxane (5 mL) were added (1R,3S)-3-(5-amino-1H-pyrazol-3-yl)cyclopentyl (prop-2-ylamino)methanoate (26 mg, 0.1 mmol), Pd₂(dba)₃ (9.6 mg, 0.010 mmol), t-BuXPhos (8.9 mg, 0.02 mmol) and Cs₂CO₃ (102 mg, 0.31 mmol). The reaction was stirred at 100° C. under N₂ for 2 hr. The cooled reaction mixture was diluted with EtOAc and water. The organic layer was separated, washed with brine and concentrated in vacuo. The residue was purified using silica gel column chromatography eluted with methanol in dichloroform (gradient: 0-10%) to afford the title compound (1R,3S)-3-[5-({1-[(4-methoxyphenyl)methyl]-2,2-dioxo-1,3-dihydro-2λ6-benzo[c][1,2]thiazol-5-yl}amino)-1H-pyrazol-3-yl]cyclopentyl (prop-2-ylamino)methanoate (20 mg, 0.04 mmol, 36%) as a yellow solid. LCMS: m/z 539.65 [M+H]⁺.

Step D: (1R,3S)-3-{5-[(2,2-dioxo-1,3-dihydro-2λ6-benzo[c][1,2]thiazol-5-yl)amino]-2H-pyrazol-3-yl}cyclopentyl (prop-2-ylamino)methanoate To a solution of (1R,3S)-3-[5-({1-[(4-methoxyphenyl)methyl]-2,2-dioxo-1,3-dihydro-2λ6-benzo[c][1,2]thiazol-5-yl}amino)-1H-pyrazol-3-yl]cyclopentyl (prop-2-ylamino)methanoate (20 mg, 0.04 mmol) in TFA (5 mL) was added trifluoromethanesulfonic acid (0.002 mL, 0.018 mmol). The reaction was stirred at room temperature for 2 h. The reaction was concentrated in vacuo. The residue was purified by prep-HPLC (C18, 0-70% acetonitrile in H₂O) to afford the title compound (1R,3S)-3-{5-[(2,2-dioxo-1,3-dihydro-26-benzo[c][1,2]thiazol-5-yl)amino]-2H-pyrazol-3-yl}cyclopentyl (prop-2-ylamino)methanoate (2.4 mg, 0.006 mmol, 15%) as a white solid. LCMS: m/z 419.50 [M+H]⁺. ¹H NMR (400 MHz, DMSO) δ 11.64 (s, 1H), 9.75 (s, 1H), 8.19 (s, 1H), 7.37 (s, 1H), 7.15 (d, J=7.2 Hz, 1H), 6.95 (d, J=8.4 Hz, 1H), 6.71 (d, J=8.4 Hz, 1H), 5.57 (s, 1H), 4.99 (s, 1H), 4.44 (s, 2H), 3.63-3.53 (m, 1H), 3.06-2.99 (m, 1H), 2.49-2.41 (m, 1H), 2.04-1.95 (m, 1H), 1.93-1.84 (m, 1H), 1.75-1.65 (m, 2H), 1.62-1.52 (m, 1H), 1.03 (d, J=6.4 Hz, 6H). Example 46 (1R,3S)-3-(3-((1,1-dioxido-2,3-dihydrobenzo[d]isothiazol-4-yl)amino)-1H-pyrazol-5-yl)cyclopentyl isopropylcarbamate

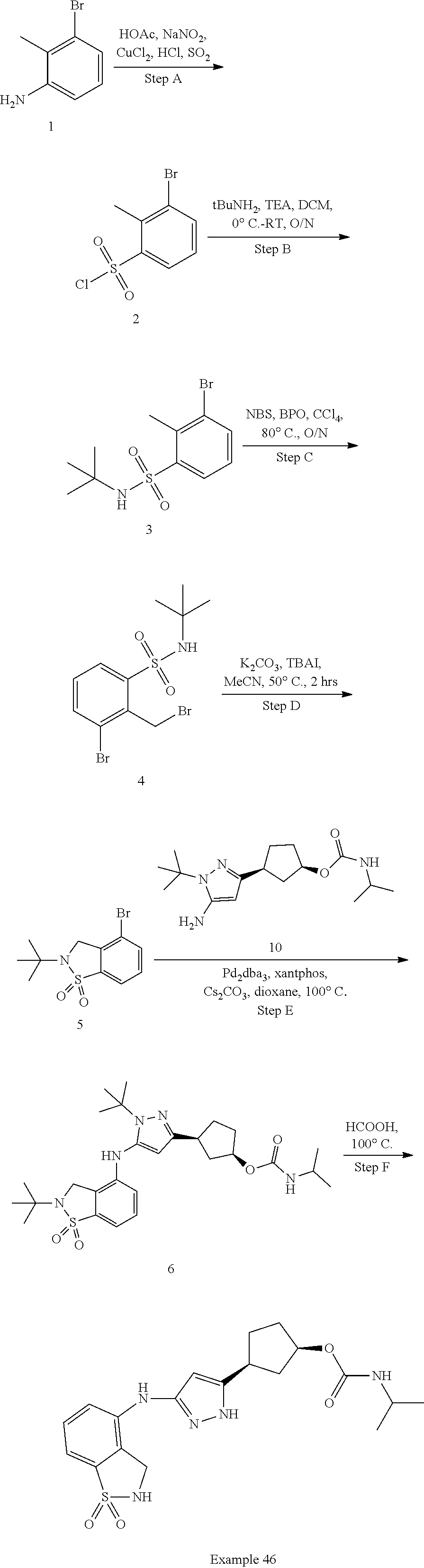

Step A: 3-bromo-2-methylbenzenesulfonyl chloride To a solution of 3-bromo-2-methylaniline (3.31 mL, 26.9 mmol) in MeCN (100 mL) at 0° C. was added con·HCl (21.0 mL) followed by AcOH (21.0 mL, 366 mmol) and a solution of NaNO₂ (2.20 g, 31.9 mmol) in H₂O (6 mL) at 0° C. The reaction mixture was purged with SO₂ gas for 15 min and a solution of CuCl₂ (4.55 g, 45.5 mmol) in H₂O (6 mL) was added dropwise at 0° C. The reaction mixture was allowed to warm to room temperature and stirred for 16 hrs. TLC showed the reaction had been completed. The reaction mixture was concentrated, diluted with 100 mL of water and extracted with 300 mL of EA. The organic extracts were washed with 100 mL of sat. aqueous sodium bicarbonate solution, 100 mL of brine, dried over sodium sulfate and concentrated to afford crude 3-bromo-2-methylbenzenesulfonyl chloride (5.50 g, 20.4 mmol, 75.9%) as a yellow oil. No Ms.

Step B: 3-bromo-N-(tert-butyl)-2-methylbenzenesulfonamide To a solution of 3-bromo-2-methylbenzenesulfonyl chloride (5.50 g, 20.4 mmol) in DCM (100 mL) at 0° C. was added triethylamine (4.25 mL, 30.6 mmol), followed by tert-butylamine (3.24 mL, 30.6 mmol). The reaction mixture was allowed to warm to room temperature and stirred for 16 h. The mixture was diluted with 100 mL of water and extracted with 300 mL of DCM. The organic extracts were washed with 100 mL of water, 100 mL of brine, dried over sodium sulfate and concentrated to afford 3-bromo-N-(tert-butyl)-2-methylbenzenesulfonamide (5.20 g, 17.0 mmol, 83.2%) as a yellow solid. LCMS: ESI m/z 304.1/306.1 [M−H]⁺. ¹H NMR (400 MHz, DMSO) δ 7.95 (d, J=8.0, 1H), 7.86 (d, J=7.6 Hz, 1H), 7.70 (s, 1H), 7.32 (dd, J=8.0, 7.6 Hz, 1H), 2.67 (s, 3H), 1.11 (s, 9H).

Step C: 3-bromo-2-(bromomethyl)-N-(tert-butyl)benzenesulfonamide To a solution of 3-bromo-N-(tert-butyl)-2-methylbenzenesulfonamide (5.00 g, 16.3 mmol) in CCl₄ (100 mL) was added NBS (5.81 g, 32.7 mmol) followed by benzoyl peroxide (0.790 g, 3.3 mmol). The mixture was then heated at 80° C. for 16 hrs. The reaction mixture was allowed to cool to room temperature, diluted with 100 mL of water and extracted with 300 mL of DCM. The combined organic extracts were washed with 100 mL of water, 100 mL of brine, dried over sodium sulfate and concentrated. The residue was purified by silica gel chromatography (eluting with 0-15% EA in PE) to afford 3-bromo-2-(bromomethyl)-N-(tert-butyl)benzenesulfonamide (6.00 g, 15.6 mmol, 95.4%) as a yellow solid. No MS. ¹H NMR (400 MHz, DMSO) δ 8.05 (d, J=8.0 Hz, 1H), 8.03-7.98 (m, 2H), 7.56-7.49 (m, 1H), 5.09 (brs, 2H), 1.23 (s, 9H).

Step D: 4-bromo-2-(tert-butyl)-2,3-dihydrobenzo[d]isothiazole 1,1-dioxide To a solution of 3-bromo-2-(bromomethyl)-N-(tert-butyl)benzenesulfonamide (3.00 g, 7.8 mmol) in MeCN (150 mL) was added K₂CO₃ (2.15 g, 15.6 mmol) followed by tetrabutylammonium iodide (0.580 g, 1.56 mmol). The reaction mixture was heated to 50° C. for 2 hrs. The reaction mixture was allowed to cool to room temperature and concentrated. The residue was resuspended in 50 mL of water and extracted with 150 mL of EtOAc. The combined organic extracts were washed with 50 mL of water, 50 mL of brine, dried over sodium sulfate and concentrated. The residue was purified by silica gel chromatography (eluting with 0-15% EtOAc in PE) to afford 4-bromo-2-(tert-butyl)-2,3-dihydrobenzo[d]isothiazole 1,1-dioxide (2.00 g, 6.58 mmol, 84%) as a faint yellow solid. LCMS: ESI m/z 248.1/250.1 [M+H-tBu]⁺. ¹H NMR (400 MHz, DMSO) δ 7.96 (d, J=7.6 Hz, 1H), 7.84 (d, J=7.8 Hz, 1H), 7.57 (dd, J=7.6, 7.8 Hz, 1H), 4.44 (s, 2H), 1.49 (s, 9H).

Step E: (1R,3S)-3-(1-(tert-butyl)-5-((2-(tert-butyl)-1,1-dioxido-2,3-dihydrobenzo[d]isothiazol-4-yl)amino)-1H-pyrazol-3-yl)cyclopentyl isopropylcarbamate To a solution of 4-bromo-2-(tert-butyl)-2,3-dihydrobenzo[d]isothiazole 1,1-dioxide (130 mg, 0.43 mmol) in dioxane (5 mL) was added (1R,3S)-3-(5-amino-1-(tert-butyl)-1H-pyrazol-3-yl)cyclopentyl isopropylcarbamate (80.0 mg, 0.259 mmol), Cs₂CO₃ (418 mg, 1.28 mmol), xantphos (49.5 mg, 0.09 mmol) and Pd₂(dba)₃ (39.1 mg, 0.04 mmol). The reaction mixture was stirred at 100° C. under N₂ overnight. The reaction was completed based ony LCMS. The cooled reaction mixture was dissolved in EA (50 mL), washed with H₂O (20 mL) and brine, dried over sodium sulfate and concentrated. The residue was purified by silica gel chromatography (eluting with 20-100% EA in PE) to afford (1R,3S)-3-(1-(tert-butyl)-5-((2-(tert-butyl)-1,1-dioxido-2,3-dihydrobenzo[d]isothiazol-4-yl)amino)-1H-pyrazol-3-yl)cyclopentyl isopropylcarbamate (150 mg, 0.282 mmol, 66.0%) as a yellow solid. LCMS: ESI m/z 532.72 [M+H]⁺.

Step F: (1R,3S)-3-(3-((1,1-dioxido-2,3-dihydrobenzo[d]isothiazol-4-yl)amino)-1H-pyrazol-5-yl)cyclopentyl isopropylcarbamate A solution of (1R,3S)-3-(1-(tert-butyl)-5-((2-(tert-butyl)-1,1-dioxido-2,3-dihydrobenzo[d]isothiazol-4-yl)amino)-1H-pyrazol-3-yl)cyclopentyl isopropylcarbamate (75.0 mg, 0.141 mmol) in formic acid (2 mL) was stirred at 100° C. for 5 hrs. LCMS showed the reaction was completed. The reaction mixture was concentrated and purified by silica gel chromatography (eluting with 0-12% MeOH in DCM) and pre-HPLC (C18, 15˜ 95% ACN in H₂O with 0.1% TFA) to afford (1R,3S)-3-(3-((1,1-dioxido-2,3-dihydrobenzo[d]isothiazol-4-yl)amino)-1H-pyrazol-5-yl)cyclopentyl isopropylcarbamate (9 mg, 0.02 mmol, 15%) as a white solid. LCMS: ESI m/z 420.5 [M+H]⁺. ¹H NMR (400 MHz, DMSO) δ 8.08 (s, 1H), 7.93 (d, J=8.0 Hz, 1H), 7.69 (s, 1H), 7.40 (t, J=8.0 Hz, 1H), 7.22 (s, 1H), 7.10-7.08 (m, 1H), 6.97-6.90 (m, 1H), 5.78 (s, 1H), 4.99 (s, 1H), 4.28 (s, 2H), 3.10-3.01 (m, 1H), 2.49-2.40 (m, 1H), 2.06-1.86 (m, 2H), 1.79-1.58 (m, 3H), 1.07-0.98 (m, 6H).

### Example 47: (1R,3S)-3-(3-((2-methyl-1,1-dioxido-2,3-dihydrobenzo[d]isothiazol-4-yl)amino)-1H-pyrazol-5-yl)cyclopentyl isopropylcarbamate

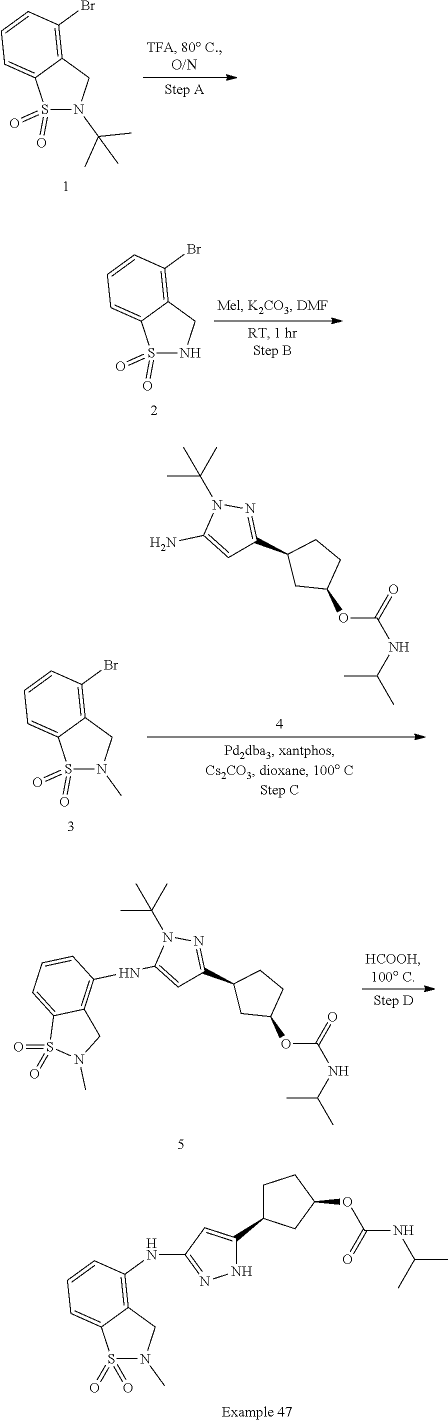 [SMILES: CC(C)(C)N1Cc2c(Br)cccc2S1(=O)=O ; O=S1(=O)NCc2c(Br)cccc21 ; CN1Cc2c(Br)cccc2S1(=O)=O ; CC(C)NC(=O)O[C@@H]1CC[C@H](c2cc(N)n(C(C)(C)C)n2)C1 ; CC(C)NC(=O)O[C@@H]1CC[C@H](c2cc(Nc3cccc4c3CN(C)S4(=O)=O)n(C(C)(C)C)n2)C1 ; CC(C)NC(=O)O[C@@H]1CC[C@H](c2cc(Nc3cccc4c3CN(C)S4(=O)=O)n[nH]2)C1]

Step A: 4-bromo-2,3-dihydrobenzo[d]isothiazole 1,1-dioxide A solution of 4-bromo-2-(tert-butyl)-2,3-dihydrobenzo[d]isothiazole 1,1-dioxide (300 mg, 0.986 mmol) in TFA (3 mL) was stirred at 80° C. overnight. LCMS showed the reaction was completed. The reaction mixture was concentrated to afford 4-bromo-2,3-dihydrobenzo[d]isothiazole 1,1-dioxide (240 mg, 0.967 mmol, 98.1%) as a reddish brown solid. LCMS: ESI m/z 248.09/250.9 [M+H]⁺.

Step B: 4-bromo-2-methyl-2,3-dihydrobenzo[d]isothiazole 1,1-dioxide To a solution of 4-bromo-2,3-dihydrobenzo[d]isothiazole 1,1-dioxide (240 mg, 0.967 mmol) in DMF (10 mL) was added K₂CO₃ (501 mg, 3.63 mmol) followed by Mel (0.226 mL, 3.63 mmol). The reaction mixture was stirred at room temperature overnight. LCMS showed the reaction had been completed. The reaction mixture was diluted with 10 mL of water and extracted with 50 mL of EA. The combined organic extracts were washed with water and brine, dried over sodium sulfate and concentrated. The residue was purified by silica gel chromatography (eluting with 0-35% EA in PE) to afford 4-bromo-2-methyl-2,3-dihydrobenzo[d]isothiazole 1,1-dioxide (190 mg, 0.725 mmol, 59.9%) as a faint yellow solid. LCMS: ESI m/z 262.12/264.12 [M+H]⁺. ¹H NMR (400 MHz, DMSO) δ 8.06 (d, J=7.6 Hz, 1H), δ8.02 (d, J=8.0 Hz, 1H), 7.65 (dd, J=7.6, 8.0 Hz, 1H), 4.43 (s, 2H), 2.93 (s, 3H).

Step C: (1R,3S)-3-(1-(tert-butyl)-5-((2-methyl-1,1-dioxido-2,3-dihydrobenzo[d]isothiazol-4-yl)amino)-1H-pyrazol-3-yl)cyclopentyl isopropylcarbamate To a solution of 4-bromo-2-methyl-2,3-dihydrobenzo[d]isothiazole 1,1-dioxide (95.0 mg, 0.362 mmol) in dioxane (5 mL) was added (1R,3S)-3-(5-amino-1-(tert-butyl)-1H-pyrazol-3-yl)cyclopentyl isopropylcarbamate (55.0 mg, 0.178 mmol), Cs₂CO₃ (174 mg, 0.535 mmol), Pd₂(dba)₃ (16.3 mg, 0.0180 mmol) and xantphos (20.6 mg, 0.0360 mmol). The reaction mixture was stirred at 100° C. under N₂ overnight. The reaction was complete detected by LCMS. The cooled reaction mixture was dissolved in EA (50 mL), washed with H₂O (20 mL) and brine, dried over sodium sulfate and concentrated. The residue was purified by silica gel chromatography (eluting with 45-80% EA in PE) to afford (1R,3S)-3-(1-(tert-butyl)-5-((2-methyl-1,1-dioxido-2,3-dihydrobenzo[d]isothiazol-4-yl)amino)-1H-pyrazol-3-yl)cyclopentyl isopropylcarbamate (65.0 mg, 0.133 mmol, 75%) as a yellow solid. LCMS: ESI m/z 490.63 [M+H]⁺.

Step D: (1R,3S)-3-(3-((2-methyl-1,1-dioxido-2,3-dihydrobenzo[d]isothiazol-4-yl)amino)-1H-pyrazol-5-yl)cyclopentyl isopropylcarbamate A solution of (1R,3S)-3-(1-(tert-butyl)-5-((2-methyl-1,1-dioxido-2,3-dihydrobenzo[d]isothiazol-4-yl)amino)-1H-pyrazol-3-yl)cyclopentyl isopropylcarbamate (65.0 mg, 0.133 mmol) in formic acid (2 mL) was stirred at 100° C. for 5 hrs. LCMS showed the reaction was completed. The cooled reaction mixture was concentrated and purified by silica gel chromatography (eluting with 0-12% MeOH in DCM) and pre-HPLC (C18, 10˜ 95% ACN in H₂O with 0.1% TFA) to afford (1R,3S)-3-(3-((2-methyl-1,1-dioxido-2,3-dihydrobenzo[d]isothiazol-4-yl)amino)-1H-pyrazol-5-yl)cyclopentyl isopropylcarbamate (11.4 mg, 0.0260 mmol, 20%) as a white solid. LCMS: ESI m/z 434.53 [M+H]⁺. ¹H NMR (400 MHz, DMSO) δ 8.16 (brs, 1H), 7.98 (d, J=8.0 Hz, 1H), 7.43 (dd, J=8.0, 7.6 Hz, 1H), 7.15 (d, J=7.6 Hz, 1H), 6.96-6.90 (m, 1H), 5.78 (s, 1H), 5.00 (brs, 1H), 4.28 (s, 2H), 3.09-3.04 (m, 1H), 2.83 (s, 3H), 2.48-2.42 (m, 1H), 2.08-1.90 (m, 2H), 1.89-1.76 (m, 2H), 1.75-1.54 (m, 2H), 1.04-1.01 (m, 6H).

### Example 48: (1R,3S)-3-(3-((2,2-dioxido-1,3-dihydrobenzo[c]isothiazol-4-yl)amino)-1H-pyrazol-5-yl)cyclopentyl isopropylcarbamate

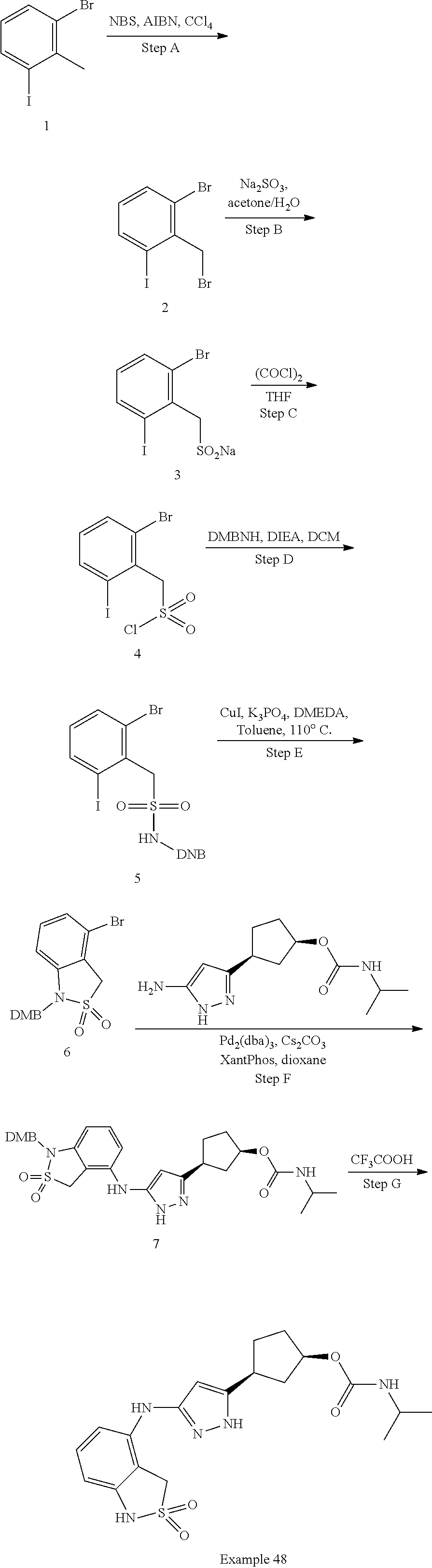

Step A: 1-bromo-2-(bromomethyl)-3-iodobenzene To a solution of 1-bromo-3-iodo-2-methylbenzene (5.6 g, 18.8 mmol) in CCl₄ (60 mL) were added NBS (4.03 g, 22.6 mmol) and AIBN (310 mg, 1.88 mmol). The reaction mixture was stirred at 80° C. overnight. The cooled reaction mixture was diluted with H₂O, extracted with DCM, washed with brine, dried by Na₂SO₄ and concentrated. The residue was purified using silica gel column chromatography eluting with EA in PE [Gradient: 0-10%] afford the title compound 1-bromo-2-(bromomethyl)-3-iodobenzene (3.5 g, 9.31 mmol, 49.3%) as a white solid. No MS. ¹HNMR (400 MHz, DMSO) δ 7.99 (d, J=8.0 Hz, 1H), 7.73 (d, J=7.6 Hz, 1H), 6.97 (dd, J=7.6, 8.0 Hz, 1H), 4.89 (brs, 2H).

Step B: sodium (2-bromo-6-iodophenyl)methanesulfonate To a solution of 1-bromo-2-(bromomethyl)-3-iodobenzene (1500 mg, 3.99 mmol) in acetone (15 mL) and H₂O (5 mL) was added Na₂SO₃ (653 mg, 5.18 mmol). The reaction mixture was stirred 90° C. overnight. The cooled reaction mixture was concentrated to afford the title compound ({[(2-bromo-6-iodophenyl)methyl]dioxo-λ⁶-sulfanyl}oxy)sodium (2200 mg crude) as a white solid. No MS

Step C: (2-bromo-6-iodophenyl)methanesulfonyl chloride To a solution of ({[(2-bromo-6-iodophenyl)methyl]dioxo-λ⁶-sulfanyl}oxy)sodium (2200 mg, 5.51 mmol) in THF (20 mL) were added DMF (0.128 mL, 1.65 mmol) and 2-chloro-2-oxoacetyl chloride (4.73 mL, 55.1 mmol) at −20° C. The reaction mixture was stirred−20° C. for 3 h. The reaction mixture was diluted with ice water, extracted with EA, washed with brine, dried by Na₂SO₄ and concentrated to afford the title compound (2-bromo-6-iodophenyl)methanesulfonyl chloride (2000 mg crude) as a white solid. No MS

Step D: 1-(2-bromo-6-iodophenyl)-N-(2,4-dimethoxybenzyl)methanesulfonamide. To a solution of (2,4-dimethoxyphenyl)methanamine (1014 mg, 6.06 mmol) in DCM (20 mL) were added DIEA (1.672 mL, 10.1 mmol) and (2-bromo-6-iodophenyl)methanesulfonyl chloride (2000 mg, 5.05 mmol). The reaction mixture was stirred RT for 2 h. The reaction mixture was diluted with H₂O, extracted with DCM, washed with brine, dried by Na₂SO₄ and concentrated. The residue was purified using silica gel column chromatography eluting with EtOAc in PE [Gradient: 0-20%] to afford the title compound 1-(2-bromo-6-iodophenyl)-N-[(2,4-dimethoxyphenyl)methyl]methanesulfonamide (1000 mg, 1.90 mmol, 38%) as a white solid. No MS. ¹HNMR (400 MHz, DMSO) δ 7.95 (dd, J=8.0, 1.1 Hz, 1H), 7.78 (dd, J=8.0, 4.0 Hz, 1H), 7.74-7.67 (m, 1H), 7.25 (d, J=8.4 Hz, 1H), 6.98 (dd, J=8.0, 8.4 Hz, 1H), 6.59-6.47 (m, 2H), 4.72 (brs, 2H), 4.15 (d, J=6.0 Hz, 2H), 3.80 (s, 3H), 3.75 (s, 3H).

Step E: 4-bromo-1-(2,4-dimethoxybenzyl)-1,3-dihydrobenzo[c]isothiazole 2,2-dioxide To a solution of 1-(2-bromo-6-iodophenyl)-N-[(2,4-dimethoxyphenyl)methyl]methanesulfonamide (500 mg, 0.950 mmol) in toluene (5 mL) were added CuI (72.3 mg, 0.380 mmol), K₃PO₄ (605 mg, 2.85 mmol) and 2,5-diazahexane (67 mg, 0.760 mmol). The reaction mixture was stirred at 110° C. under N₂ overnight. The cooled reaction mixture was diluted with H₂O, extracted with EA, washed with brine, dried by Na₂SO₄ and concentrated. The residue was purified using silica gel column chromatography eluting with EA in PE [Gradient: 0-50%] afford the title compound 4-bromo-1-[(2,4-dimethoxyphenyl)methyl]-1,3-dihydro-2λ⁶-benzo[c][1,2]thiazole-2,2-dione (300 mg, 0.753 mmol, 79.2%) as a white solid. LCMS: ESI m/z 398 [M+H]⁺

Step F: (1R,3S)-3-(5-((1-(2,4-dimethoxybenzyl)-2,2-dioxido-1,3-dihydrobenzo[c]isothiazol-4-yl)amino)-1H-pyrazol-3-yl)cyclopentyl isopropylcarbamate To a solution of (1R,3S)-3-(5-amino-1H-pyrazol-3-yl)cyclopentyl (prop-2-ylamino)methanoate (150 mg, 0.594 mmol) in dioxane (5 mL) were added 4-bromo-1-[(2,4-dimethoxyphenyl)methyl]-1,3-dihydro-2λ⁶-benzo[c][1,2]thiazole-2,2-dione (355 mg, 0.892 mmol), Cs₂CO₃ (387 mg, 1.18 mmol), Xantphos (68.8 mg, 0.119 mmol) and Pd₂(dba)₃ (54.4 mg, 0.059 mmol). The reaction mixture was stirred at 100° C. under N₂ overnight. The cooled reaction mixture was diluted with H₂O, extracted with EA, washed with brine, dried by Na₂SO₄ and concentrated. The residue was purified using silica gel column chromatography eluting with EA in PE [Gradient: 0-50%] afford the title compound (1R,3S)-3-[5-({1-[(2,4-dimethoxyphenyl)methyl]-2,2-dioxo-1,3-dihydro-2λ⁶-benzo[c][1,2]thiazol-4-yl}amino)-1H-pyrazol-3-yl]cyclopentyl (prop-2-ylamino)methanoate (30 mg, 0.053 mmol, 9%) as a white solid. LCMS: ESI m/z 571[M+H]⁺.

Step G: (1R,3S)-3-(3-((2,2-dioxido-1,3-dihydrobenzo[c]isothiazol-4-yl)amino)-1H-pyrazol-5-yl)cyclopentyl isopropylcarbamate A solution of (1R,3S)-3-[5-({1-[(2,4-dimethoxyphenyl)methyl]-2,2-dioxo-1,3-dihydro-2λ⁶-benzo[c][1,2]thiazol-4-yl}amino)-1H-pyrazol-3-yl]cyclopentyl (prop-2-ylamino)methanoate (30 mg, 0.053 mmol) in CF₃COOH (2 mL) was stirred at RT for 4 h. The reaction mixture was diluted with H₂O, extracted with EA, washed with brine, dried by Na₂SO₄ and concentrated. The residue was purified by prep-HPLC to afford the title compound (1R,3S)-3-{5-[(2,2-dioxo-1,3-dihydro-2λ⁶-benzo[c][1,2]thiazol-4-yl)amino]-1H-pyrazol-3-yl}cyclopentyl (prop-2-ylamino)methanoate (0.8 mg, 0.002 mmol, 3.62%) as a white solid. LCMS: ESI m/z 420[M+H]⁺. ¹HNMR(400 MHz, MeOD) δ 7.15 (dd, J=8.0, 8.4 Hz, 1H), 6.93 (d, J=8.4 Hz, 1H), 6.43 (d, J=8.0 Hz, 1H), 5.09 (s, 1H), 4.26 (s, 2H), 3.72-64 (m, 1H), 2.56-2.50 (m, 1H), 2.24-2.14 (m, 1H), 2.05-1.80 (m, 5H), 1.14-1.07 (m, 6H).

### Example 49: Synthesis of (1s,4s)-4-{5-[(1,1-dioxo-2,3-dihydro-1λ<sup>6</sup>-benzo[b]thiophen-4-yl)amino]-2H-pyrazol-3-yl}cyclohexyl (prop-2-ylamino)methanoate

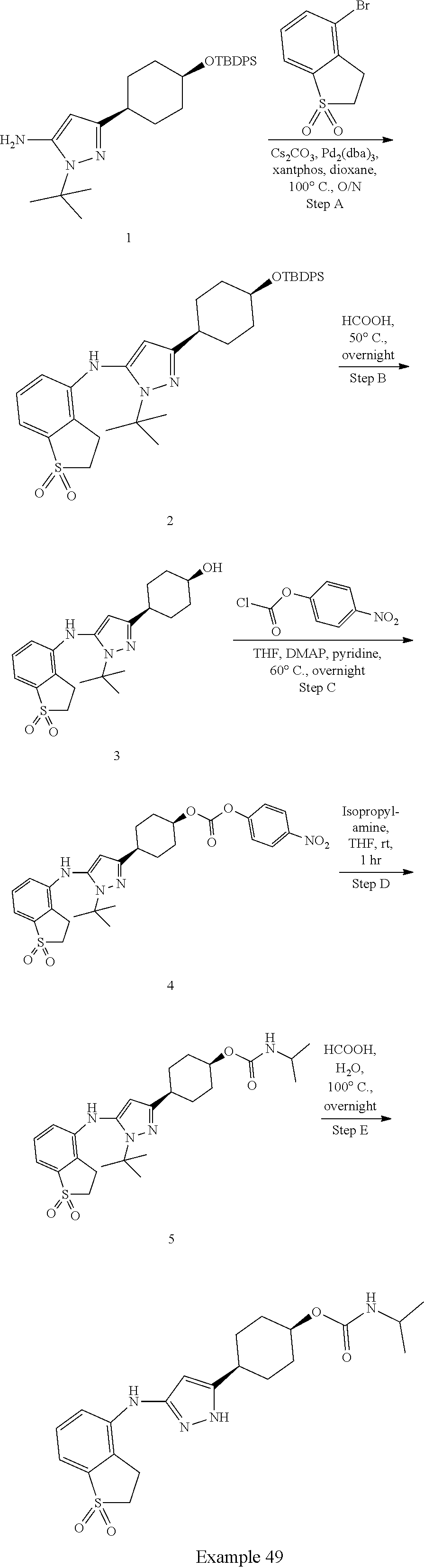

Step A: 4-((1-(tert-butyl)-3-((1s,4s)-4-((tert-butyldiphenylsilyl)oxy)cyclohexyl)-1H-pyrazol-5-yl)amino)-2,3-dihydrobenzo[b]thiophene 1,1-dioxide To a solution of 5-[(1s,4s)-4-{[(2-methylprop-2-yl)diphenylsilyl]oxy}cyclohexyl]-2-(2-methylprop-2-yl)pyrazol-3-amine (500 mg, 1.051 mmol) in dioxane (15 mL) were added 4-bromo-2,3-dihydro-16-benzothiophene-1,1-dione (311.6 mg, 1.261 mmol), CS₂CO₃ (1.02 g, 3.153 mmol), Xant-PHOS (60.81 mg, 0.s mmol) and Pd₂(dba)₃ (96.2 mg, 0.105 mmol). The reaction mixture was stirred at 100° C. under N₂ overnight. The cooled reaction mixture was concentrated, the residue was purified using silica gel column chromatography eluting with ethyl acetate in petroleum ether (gradient: 0-100%) to afford 4-((1-(tert-butyl)-3-((1s,4s)-4-((tert-butyldiphenylsilyl)oxy)cyclohexyl)-1H-pyrazol-5-yl)amino)-2,3-dihydrobenzo[b]thiophene 1,1-dioxide (650 mg, 1.01 mmol, 96%) as a yellow solid. LCMS: 642 [M+H]⁺.

Step B: 4-((1-(tert-butyl)-3-((1s,4s)-4-hydroxycyclohexyl)-1H-pyrazol-5-yl)amino)-2,3-dihydrobenzo[b]thiophene 1,1-dioxide A solution of 4-((1-(tert-butyl)-3-((1s,4s)-4-((tert-butyldiphenylsilyl)oxy)cyclohexyl)-1H-pyrazol-5-yl)amino)-2,3-dihydrobenzo[b]thiophene 1,1-dioxide (650 mg, 1.255 mmol) in formic acid (10 mL) was stirred at 50° C. overnight. The cooled reaction was diluted with EA and water. The organic layer was separated, washed with brine and concentrated. The residue was purified using silica gel column chromatography eluting with ethyl acetate in petroleum ether (gradient: 0-50%) to afford 4-((1-(tert-butyl)-3-((1s,4s)-4-hydroxycyclohexyl)-1H-pyrazol-5-yl)amino)-2,3-dihydrobenzo[b]thiophene 1,1-dioxide (200 mg, 0.496 mmol, 40%) as a white solid. LCMS: 404 [M+H]⁺.

Step C: (1s,4s)-4-(1-(tert-butyl)-5-((1,1-dioxido-2,3-dihydrobenzo[b]thiophen-4-yl)amino)-1H-pyrazol-3-yl)cyclohexyl (4-nitrophenyl) carbonate To a solution of 4-((1-(tert-butyl)-3-((1s,4s)-4-hydroxycyclohexyl)-1H-pyrazol-5-yl)amino)-2,3-dihydrobenzo[b]thiophene 1,1-dioxide (150 mg, 0.37 mmol) in pyridine (5 mL) were added DMAP (5 mg, 0.04 mmol) and 4-nitrophenyl chloromethanoate (113 mg, 0.56 mmol). The resulting solution was stirred at 60° C. overnight. The cooled reaction was diluted with EA and water. The organic layer was separated, washed with brine, dried over Na₂SO₄, filtered and concentrated. The residue was purified by silica gel column chromatography eluted with ethyl acetate in petroleum ether (gradient: 0-50%) to afford (1s,4s)-4-(1-(tert-butyl)-5-((1,1-dioxido-2,3-dihydrobenzo[b]thiophen-4-yl)amino)-1H-pyrazol-3-yl)cyclohexyl (4-nitrophenyl) carbonate (75 mg, 0.12 mmol, 33%) as a gray solid. LCMS: 569 [M+H]⁺.

Step D: (1s,4s)-4-(1-(tert-butyl)-5-((1,1-dioxido-2,3-dihydrobenzo[b]thiophen-4-yl)amino)-1H-pyrazol-3-yl)cyclohexyl isopropylcarbamate To a solution of (1s,4s)-4-(1-(tert-butyl)-5-((1,1-dioxido-2,3-dihydrobenzo[b]thiophen-4-yl)amino)-1H-pyrazol-3-yl)cyclohexyl (4-nitrophenyl) carbonate (100 mg, 0.176 mmol) in THF (2 mL) were added propan-2-amine (2 mL, 23.3 mmol). The reaction mixture was stirred at room temperature for 1 hr. The residue was purified using silica gel column chromatography eluting with ethyl acetate in petroleum ether (gradient: 0-100%) to afford the (1s,4s)-4-(1-(tert-butyl)-5-((1,1-dioxido-2,3-dihydrobenzo[b]thiophen-4-yl)amino)-1H-pyrazol-3-yl)cyclohexyl isopropylcarbamate (59 mg, 0.121 mmol, 69%) as a yellow solid. LCMS: 489 [M+H]⁺.

Step E: (1s,4s)-4-(3-((1,1-dioxido-2,3-dihydrobenzo[b]thiophen-4-yl)amino)-1H-pyrazol-5-yl)cyclohexyl isopropylcarbamate A mixture of (1s,4s)-4-(1-(tert-butyl)-5-((1,1-dioxido-2,3-dihydrobenzo[b]thiophen-4-yl)amino)-1H-pyrazol-3-yl)cyclohexyl isopropylcarbamate (59 mg, 0.121 mmol) in H₂O (1 mL) and formic acid (5 mL) was stirred at 100° C. overnight. The cooled reaction mixture was concentrated. The residue was purified by prep-HPLC (C18, 0-70% acetonitrile in H₂O) to afford the title compound (1s,4s)-4-(3-((1,1-dioxido-2,3-dihydrobenzo[b]thiophen-4-yl)amino)-1H-pyrazol-5-yl)cyclohexyl isopropylcarbamate (10 mg, 0.023 mmol, 19%) as a white solid. LCMS: 433[M+H]⁺. ¹H NMR (400 MHz, DMSO) δ 8.01 (s, 2H), 7.37 (t, J=7.6 Hz, 1H), 7.02 (d, J=7.6 Hz, 1H), 6.87 (s, 1H), 5.79 (s, 1H), 4.75 (s, 1H), 3.61 (s, 2H), 3.21 (t, J=6.4 Hz, 2H), 2.73-2.64 (m, 1H), 1.87-1.52 (m, 8H), 1.04 (d, J=6.4 Hz, 6H).

### Example 50: Synthesis of (1R,3S)-3-{5-[(7-fluoro-1,1-dioxo-2,3-dihydro-1λ<sup>6</sup>-benzo[b]thiophen-4-yl)amino]-2H-pyrazol-3-yl}cyclopentyl (prop-2-ylamino)methanoate

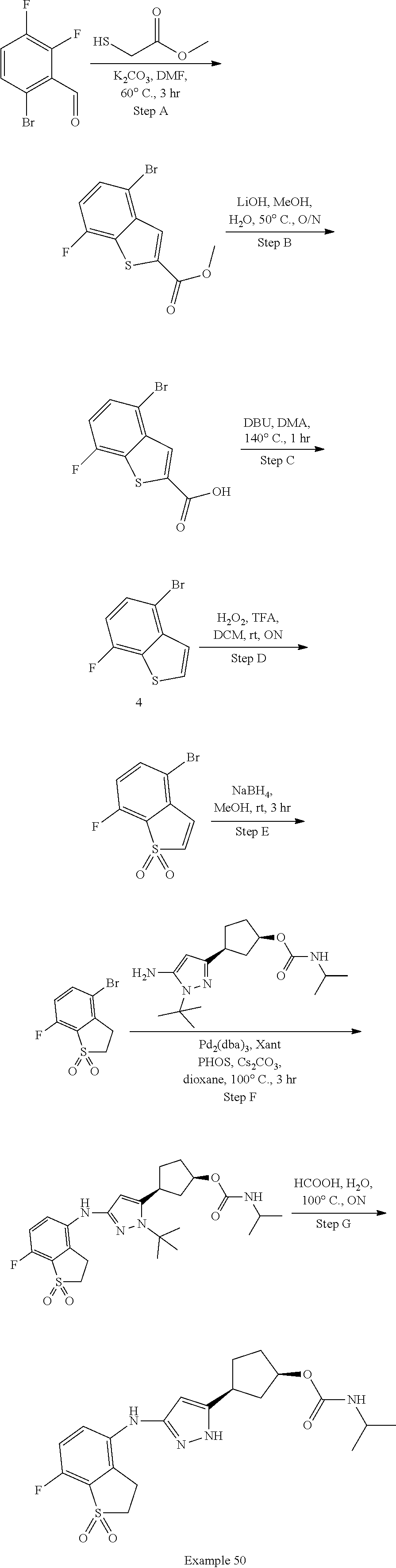

Step A: methyl 4-bromo-7-fluorobenzo[b]thiophene-2-carboxylate To a solution of 6-bromo-2,3-difluorobenzene-1-carbaldehyde (2.4 g, 10.8 mmol) in DMF (25 mL) were added methyl sulfanylacetate (1.00 mL, 10.8 mmol) and K₂CO₃ (2.9 g, 20.9 mmol). The reaction mixture was stirred at 60° C. for 3 hr. The cooled reaction mixture was diluted with EA and water. The organic layer was separated, washed with brine, dried over Na₂SO₄ and concentrated to afford the title compound methyl 4-bromo-7-fluorobenzothiophene-2-carboxylate (2.56 g, 8.85 mmol, 81.5%) as a white solid. ¹H NMR (400 MHz, DMSO) δ 8.04 (d, J=3.2 Hz, 1H), 7.78 (dd, J=8.4, 4.4 Hz, 1H), 7.44 (t, J=8.4 Hz, 1H), 3.94 (s, 3H).

Step B: 4-bromo-7-fluorobenzo[b]thiophene-2-carboxylic acid To a solution of methyl 4-bromo-7-fluorobenzothiophene-2-carboxylate (1 g, 3.45 mmol) in MeOH (10 mL) and H₂O (10 mL) were added LiOH (0.73 g, 17.29 mmol). The reaction mixture was stirred at 50° C. overnight. The cooled reaction mixture was diluted with EA and water. The organic layer was separated, washed with brine, dried over Na₂SO₄ and concentrated to afford the title compound 4-bromo-7-fluorobenzothiophene-2-carboxylic acid (940 mg, 3.41 mmol, 98.8%) as a white solid. LCMS: 275 [M+H]⁺

Step C: 4-bromo-7-fluorobenzo[b]thiophene To a solution of 4-bromo-7-fluorobenzothiophene-2-carboxylic acid (940 mg, 3.41 mmol) in DMA (10 mL) were added 2,3,4,6,7,8,9,10-octahydropyrimido[1,2-a]azepine (550 mg, 3.61 mmol). The reaction was stirred at 140° C. for 1.5 hr with microwave. The cooled reaction mixture was diluted with EA and water. The organic layer was separated, washed with brine, dried over Na₂SO₄ and concentrated to afford the title compound 4-bromo-7-fluorobenzothiophene (610 mg, 2.64 mmol) as a white solid. ¹H NMR (400 MHz, DMSO) δ 8.06 (d, J=5.2 Hz, 1H), 7.68 (dd, J=8.4, 4.4 Hz, 1H), 7.55-7.48 (m, 1H), 7.31-7.21 (m, 1H).

Step D: 4-bromo-7-fluoro-1,<sup>6</sup>-benzo[b]thiophene-1,1-dione To a solution of 4-bromo-7-fluorobenzothiophene (610 mg, 2.64 mmol) in DCM (6 mL) were added H₂O₂(1 mL, 9.8 mmol, 30% in H₂O) and TFA (1 mL, 13.5 mmol). The reaction was stirred at room temperature overnight. The reaction mixture was diluted with EA and water. The organic layer was separated, washed with further brine and concentrated in vacuo. The residue was purified using silica gel column chromatography eluting with ethyl acetate in petroleum ether (gradient: 0-50%) to afford the 4-bromo-7-fluoro-1λ⁶-benzothiophene-1,1-dione (300 mg, 1.14 mmol, 43%) as a white solid. ¹H NMR (400 MHz, DMSO) δ 8.00 (dd, J=8.6, 4.4 Hz, 1H), 7.63 (dd, J=6.8, 2.0 Hz, 1H), 7.52 (t, J=8.6 Hz, 1H).

Step E: 4-bromo-7-fluoro-2,3-dihydro-1λ<sup>6</sup>-benzo[b]thiophene-1,1-dione To a solution of 4-bromo-7-fluoro-1λ⁶-benzothiophene-1,1-dione (300 mg, 1.14 mmol) in MeOH (3 mL) were added NaBH₄ (100 mg, 2.95 mmol). The reaction was stirred at room temperature for 1 hr. The reaction was diluted with EA and water. The organic layer was separated, washed with brine, dried over Na₂SO₄ and concentrated. The residue was purified using silica gel column chromatography eluting with ethyl acetate in petroleum ether (gradient: 0-50%) to afford the 4-bromo-7-fluoro-2,3-dihydro-1λ⁶-benzothiophene-1,1-dione (110 mg, 0.415 mmol, 36%) as a white solid. LCMS: 265 [M+H]⁺.

Step F: (1R,3S)-3-(1-(tert-butyl)-3-((7-fluoro-1,1-dioxido-2,3-dihydrobenzo[b]thiophen-4-yl)amino)-1H-pyrazol-5-yl)cyclopentyl isopropylcarbamate To a solution of 4-bromo-7-fluoro-2,3-dihydro-1λ⁶-benzothiophene-1,1-dione (110 mg, 0.415 mmol) in dioxane (3 mL) were added (1R,3S)-3-[5-amino-1-(2-methylprop-2-yl)pyrazol-3-yl]cyclopentyl (prop-2-ylamino)methanoate (128 mg, 0.415 mmol), Pd₂(dba)₃ (38.00 mg, 0.041 mmol), Xant-PHOS (24.00 mg, 0.041 mmol) and Cs₂CO₃ (270 mg, 0.830 mmol). The reaction mixture was stirred at 100° C. under N₂ for 3 hr. The cooled reaction was diluted with EA and water. The organic layer was separated, washed with brine, dried over Na₂SO₄ and concentrated in vacuo. The residue was purified using silica gel column chromatography eluting with methanol in dichloroform (gradient:0-10%) to afford (1R,3S)-3-(1-(tert-butyl)-3-((7-fluoro-1,1-dioxido-2,3-dihydrobenzo[b]thiophen-4-yl)amino)-1H-pyrazol-5-yl)cyclopentyl isopropylcarbamate (114 mg, 0.261 mmol, 63%) as a black solid. LCMS: 493 [M+H]⁺.

Step G: (1R,3S)-3-{5-[(7-fluoro-1,1-dioxo-2,3-dihydro-1λ<sup>6</sup>-benzo[b]thiophen-4-yl)amino]-2H-pyrazol-3-yl}cyclopentyl (prop-2-ylamino)methanoate A solution of (1R,3S)-3-(1-(tert-butyl)-3-((7-fluoro-1,1-dioxido-2,3-dihydrobenzo[b]thiophen-4-yl)amino)-1H-pyrazol-5-yl)cyclopentyl isopropylcarbamate (114 mg, 0.231 mmol) in formic acid (5 mL) and H₂O (1 mL) was stirred at 100° C. overnight. The cooled reaction mixture was concentrated. The residue was purified by prep-HPLC (C18, 0-70% acetonitrile in H₂O) to afford the title compound (1R,3S)-3-{5-[(7-fluoro-1,1-dioxo-2,3-dihydro-1λ⁶-benzothiophen-4-yl)amino]-2H-pyrazol-3-yl}cyclopentyl (prop-2-ylamino)methanoate (10.6 mg, 0.024 mmol, 10.49%) as a white solid. LCMS: 437 [M+H]⁺. ¹HNMR (400 MHz, DMSO) δ 11.87 (s, 1H), 8.13-7.86 (m, 2H), 7.20 (t, J=8.8 Hz, 1H), 6.94 (d, J=7.2 Hz, 1H), 5.75 (s, 1H), 5.00 (s, 1H), 3.70 (t, J=6.8 Hz, 2H), 3.63-3.52 (m, 1H), 3.26-3.20 (m, 2H), 3.12-2.99 (m, 1H), 2.49-2.40 (m, 1H), 2.08-1.97 (m, 1H), 1.96-1.85 (m, 1H), 1.78-1.66 (m, 2H), 1.65-1.53 (m, 1H), 1.03 (d, J=6.4 Hz, 6H).

### Example 51: Synthesis of (1R,3S)-3-{5-[(1-methyl-2,2-dioxo-1,3-dihydro-2λ<sup>6</sup>-benzo[2,1-c][1,2]thiazol-7-yl)amino]-2H-pyrazol-3-yl}cyclopentyl (prop-2-ylamino)methanoate

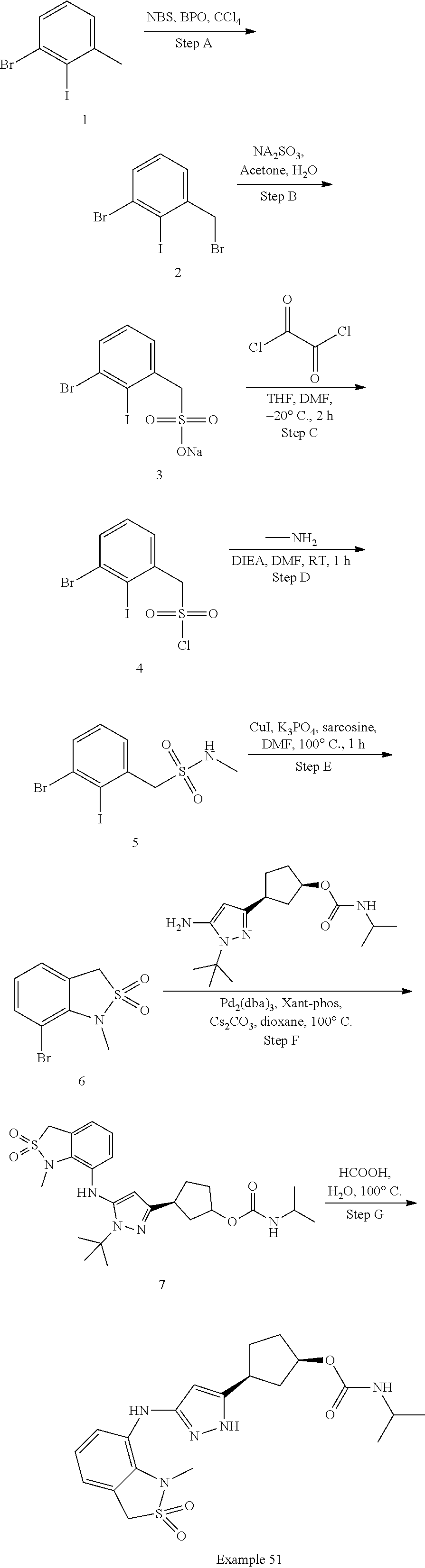

Step A. 1-bromo-3-(bromomethyl)-2-iodobenzene. To a stirred solution of 1-bromo-2-iodo-3-methylbenzene (4 g, 13 mmol) in tetrachloromethane (CCl₄, 20 mL) was added N-bromosuccinimide (NBS, 3.5 g, 19.0 mmol) and benzoyl peroxide (BPO, 0.26 g, 1.9 mmol) at rt. After stirred at reflux for 5 h, the cooled mixture was washed with saturated sodium bicarbonate solution and brine, dried with anhydrous sodium sulfate and condensed under vacuum. The residue was purified by silica gel chromatography (petrol ether: ethyl acetate=50:1 to 10:1) to get a white solid of 1-bromo-3-(bromomethyl)-2-iodobenzene (2 g, 47%) as a light oil. LCMS: m/z 375 [M+H]⁺.

Step B. ({[(3-bromo-2-iodophenyl)methyl]dioxo-λ<sup>6</sup>-sulfanyl}oxy)sodium. To a stirred solution of 1-bromo-3-(bromomethyl)-2-iodobenzene (2 g, 5.3 mmol) in actone (18 mL)/H₂O (12 mL) was added Na₂SO₃ (0.74 g, 5.8 mmol) at rt. After stirred at 90° C. for 3 h, the cooled mixture was concentrated to give crude ({[(3-bromo-2-iodophenyl)methyl]dioxo-λ⁶-sulfanyl}oxy)sodium (2 g, 5.1 mmol, 94%) as a white solid. LCMS: m/z 398 [M+H]⁻.

Step C. (3-bromo-2-iodophenyl)methanesulfonyl chloride. To a stirred solution of ({[(3-bromo-2-iodophenyl)methyl]dioxo-λ⁶-sulfanyl}oxy)sodium (1.8 g, 4.5 mmol) in THF (20 mL) was added DMF (0.1 mL) and 2-chloro-2-oxoacetyl chloride (3.8 mL, 45.1 mmol) slowly at −20° C. After stirring at −20° C. for 2 h, the mixture was poured into ice-water (20 mL) and extracted with EtOAc (10 mL*2). The combined organic phase was washed by brine, dried over Na₂SO₄, filtered and concentrated to give crude (3-bromo-2-iodophenyl)methanesulfonyl chloride (1.4 g, 3.5 mmol, 78%) as a yellow solid. LCMS: m/z 394 [M+H]⁻.

Step D. 1-(3-bromo-2-iodophenyl)-N-methylmethanesulfonamide. To a stirred solution of (3-bromo-2-iodophenyl)methanesulfonyl chloride (1.4 g, 3.5 mmol) in DMF (15 mL) was added methanamine (5.3 mL, 11 mmol) and DIPEA (0.7 mL, 3.8 mmol) slowly at rt. After stirring at rt for 1 h, the cooled mixture was poured into water (30 mL) and extracted with EtOAc (20 mL*2). The combined organic phase was washed with brine, dried with Na₂SO₄, filtered and concentrated. The residue was purified by chromatography (silica gel, 0-30%, EtOAc in PE) to give 1-(3-bromo-2-iodophenyl)-N-methylmethanesulfonamide (900 mg, 2.3 mmol, 65%) as a yellow solid. LCMS: m/z 389 [M+H]⁻.

Step E. 7-bromo-1-methyl-1,3-dihydro-2λ<sup>6</sup>-benzo[c][1,2]thiazole-2,2-dione. To a stirred solution of 1-(3-bromo-2-iodophenyl)-N-methylmethanesulfonamide (400 mg, 1.1 mmol) in DMF (30 mL) was added CuI (58 mg, 0.31 mmol), Sarcosine (55 mg, 0.61 mmol) and K₃PO₄ (1.1 g, 5.1 mmol) at rt. After stirring at 100° C. for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-40%, EtOAc in PE) to give 7-bromo-1-methyl-1,3-dihydro-2λ⁶-benzo[c][1,2]thiazole-2,2-dione (250 mg, 1.1 mmol, 96%) as a yellow solid. LCMS: m/z 262/264 [M+H]⁺.

Step F. (1R,3S)-3-{5-[(1-methyl-2,2-dioxo-1,3-dihydro-2%<sup>6</sup>-benzo[2,1-c][1,2]thiazol-7-yl)amino]-1-(2-methylprop-2-yl)pyrazol-3-yl}cyclopentyl (prop-2-ylamino)methanoate. To a stirred solution of 7-bromo-1-methyl-1,3-dihydro-2λ⁶-benzo[c][1,2]thiazole-2,2-dione (80 mg, 0.31 mmol) in dioxane (8 mL) was added (1R,3S)-3-[5-amino-1-(2-methylprop-2-yl)pyrazol-3-yl]cyclopentyl (prop-2-ylamino)methanoate (75 mg, 0.2 mmol), XANT PHOS (35 mg, 0.06 mmol), Pd₂(dba)₃ (28 mg, 0.03 mmol) and Cs₂CO₃ (298 mg, 0.92 mmol) at rt. After stirred at 100° C. under N₂ for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-50%, EtOAc in PE) to give (1R,3S)-3-{5-[(1-methyl-2,2-dioxo-1,3-dihydro-2λ⁶-benzo[2,1-c][1,2]thiazol-7-yl)amino]-1-(2-methylprop-2-yl)pyrazol-3-yl}cyclopentyl (prop-2-ylamino)methanoate (70 mg, 0.14 mmol, 47%) as a yellow oil. LCMS: m/z 490 [M+H]⁺.

Step G. (1R,3S)-3-{5-[(1-methyl-2,2-dioxo-1,3-dihydro-2λ<sup>6</sup>-benzo[2,1-c][1,2]thiazol-7-yl)amino]-2H-pyrazol-3-yl}cyclopentyl (prop-2-ylamino)methanoate. To a stirred solution of (1R,3S)-3-{5-[(1-methyl-2,2-dioxo-1,3-dihydro-2λ⁶-benzo[2,1-c][1,2]thiazol-7-yl)amino]-1-(2-methylprop-2-yl)pyrazol-3-yl}cyclopentyl (prop-2-ylamino)methanoate (70 mg, 0.14 mmol) in HCOOH (3 mL) was added H₂O (1 mL, 55 mmol) at rt. After stirring at 100° C. for 5 h, the cooled mixture was concentrated. The residue was purified by chromatography (silica gel, 0-70%, EtOAc in PE) and then prep-HPLC (C18, 40˜90% MeCN in H₂O with 0.1% HCOOH) to give (1R,3S)-3-{5-[(1-methyl-2,2-dioxo-1,3-dihydro-2λ⁶-benzo[2,1-c][1,2]thiazol-7-yl)amino]-2H-pyrazol-3-yl}cyclopentyl (prop-2-ylamino)methanoate (2.1 mg, 0.005 mmol, 3.5%) as a white solid. LCMS: m/z 434 [M+H]⁺. ¹H NMR (400 MHz, MeOD) δ 7.36 (d, J=8.4 Hz, 1H), 7.08 (dd, J=7.6, 8.4 Hz, 1H), 6.87 (d, J=7.6 Hz, 1H), 5.81 (s, 1H), 5.08 (s, 1H), 4.58 (s, 1H), 4.40 (s, 2H), 3.71-3.65 (m, 1H), 3.21-3.12 (m, 1H), 3.08 (s, 3H), 2.55-2.47 (m, 1H), 2.17-2.08 (m, 1H), 2.00-1.75 (m, 3H), 1.25-1.06 (m, 6H)

### Example 52: (1R,3S)-3-(3-((5-fluoro-1,1-dioxido-2,3-dihydrobenzo[b]thiophen-4-yl)amino)-1H-pyrazol-5-yl)cyclopentyl isopropylcarbamate

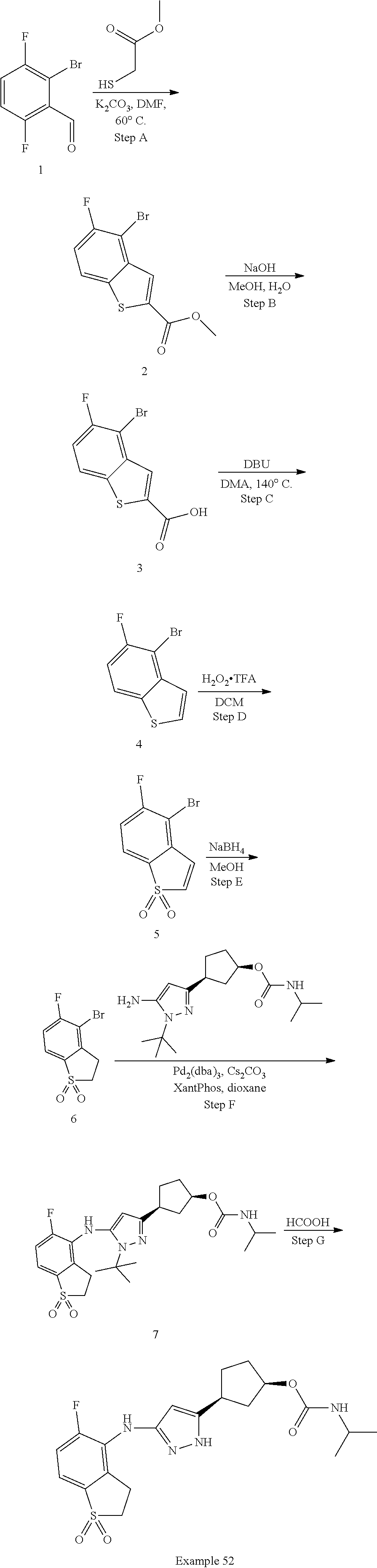

Step A: methyl 4-bromo-5-fluorobenzo[b]thiophene-2-carboxylate To a solution of 2-bromo-3,6-difluorobenzene-1-carbaldehyde (2 g, 9.05 mmol) in DMF (15 mL) were added K₂CO₃ (2.50 g, 18.1 mmol) and methyl sulfanylacetate (0.809 mL, 9.05 mmol). The reaction mixture was stirred at 60° C. under N₂ overnight. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 10%] to afford the title compound methyl 4-bromo-5-fluorobenzo[b]thiophene-2-carboxylate (1.5 g, 5.18 mmol, 57.3%) as a white solid. ¹H NMR (400 MHz, CDCl₃) δ 8.17 (s, 1H), 7.74 (dd, J=8.8, 4.4 Hz, 1H), 7.16 (d, J=1.6 Hz, 1H), 3.97 (s, 3H).

Step B: 4-bromo-5-fluorobenzothiophene-2-carboxylic acid To a solution of methyl 4-bromo-5-fluorobenzo[b]thiophene-2-carboxylate (1.5 g, 5.18 mmol) in MeOH (5 mL) was added dropwise a solution of NaOH (0.42 g, 10.3 mmol) in H₂O (5 mL). The reaction mixture was stirred at RT for 4 hr. The mixture was adjusted pH to 5 by concentrated hydrochloric acid, extracted with EA. The organic layer was separated, washed with brine, dried over Na₂SO₄, filtered and concentrated in vacuo. The residue was purified using silica gel column chromatography eluting with MeOH in DCM [Gradient: 5%] to afford the title compound {4-bromo-5-fluorobenzothiophene-2-carboxylic acid (1.3 g, 4.72 mmol, 91.1%) as a white solid. LCMS: ESI m/z 275 [M+H]⁺

Step C: 4-bromo-5-fluorobenzothiophene To a solution of 4-bromo-5-fluorobenzothiophene-2-carboxylic acid (260 mg, 0.94 mmol) in DMA (5 mL) were added DBU (0.706 mL, 4.72 mmol). The reaction mixture was stirred at 140° C. for 1 hr with microwave. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 3%] to afford the title compound 4-bromo-5-fluorobenzothiophene (160 mg, 0.692 mmol, 73.2%) as a white solid. ¹H NMR (400 MHz, CDCl₃) δ 7.61-7.53 (m, 1H), 7.51-7.42 (m, 2H), 7.17-7.11 (m, 1H).

Step D: 4-bromo-5-fluorobenzo[b]thiophene 1,1-dioxide To a stirred solution of 4-bromo-5-fluorobenzothiophene (780 mg, 3.37 mmol) in DCM (8 mL) were added H₂O₂(2 mL, 19.6 mmol, 30% in H₂O) and TFA (2 mL, 26.9 mmol) at room temperature. After stirred at room temperature for 5 h, the mixture was poured into Na₂S₂O₃ (aq) and extracted with DCM. The combined organic phase was washed with brine, dried over Na₂SO₄, filtered and concentrated. The residue was purified by chromatography eluting with EA in PE [Gradient: 10%] to give 4-bromo-5-fluorobenzo[b]thiophene 1,1-dioxide (300 mg, 1.140 mmol, 34%) as a white solid. No MS. ¹H NMR (400 MHz, CDCl₃) δ 7.65 (dd, J=8.0, 4.4 Hz, 1H), 7.40 (d, J=7.2 Hz, 1H), 7.27 (dd, J=9.6, 4.4 Hz, 1H), 6.89 (d, J=7.2 Hz, 1H).

Step E: 4-bromo-5-fluoro-2,3-dihydrobenzo[b]thiophene 1,1-dioxide To a solution of 4-bromo-5-fluorobenzo[b]thiophene 1,1-dioxide 300 mg, 1.14 mmol) in MeOH (5 mL) was added NaBH₄ (115 mg, 13.9 mmol) at 0° C. The reaction mixture was stirred at room temperature for 2 hr. The reaction was complete detected by LCMS. The residue was concentrated and dissolved in EA, washed with H₂O and brine, dried over Na₂SO₄ and concentrated. The residue was purified by silica gel chromatography eluting with EA in PE [Gradient:10%] to afford compound 4-bromo-5-fluoro-2,3-dihydrobenzo[b]thiophene 1,1-dioxide (100 mg, 0.377 mmol, 33%) as a white solid. LCMS: ESI m/z 265 [M+H]⁺. ¹H NMR (400 MHz, CDCl₃) δ 7.70 (dd, J=8.4, 4.4 Hz, 1H), 7.26 (d, J=16.4 Hz, 1H), 3.63-3.54 (m, 2H), 3.41-3.31 (m, 2H).

Step F: (1R,3S)-3-(1-(tert-butyl)-5-((5-fluoro-1,1-dioxido-2,3-dihydrobenzo[b]thiophen-4-yl)amino)-1H-pyrazol-3-yl)cyclopentyl isopropylcarbamate To a solution of (1R,3S)-3-[5-amino-1-(2-methylprop-2-yl)pyrazol-3-yl]cyclopentyl (prop-2-ylamino)methanoate (100 mg, 0.324 mmol) in dioxane (5 mL) were added 4-bromo-5-fluoro-2,3-dihydrobenzo[b]thiophene 1,1-dioxide (100 mg, 0.377 mmol), Cs₂CO₃ (211 mg, 0.648 mmol), Xant PHOS (37 mg, 0.065 mmol) and Pd₂(dba)₃ (30 mg, 0.032 mmol). The reaction mixture was stirred at 100° C. under N₂ overnight. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 45%] to afford the title compound (1R,3S)-3-(1-(tert-butyl)-5-((5-fluoro-1,1-dioxido-2,3-dihydrobenzo[b]thiophen-4-yl)amino)-1H-pyrazol-3-yl)cyclopentyl isopropylcarbamate (90 mg, 0.183 mmol, 56.3%) as a white solid. LCMS: ESI m/z 493 [M+H]⁺.

Step G: (1R,3S)-3-(3-((5-fluoro-1,1-dioxido-2,3-dihydrobenzo[b]thiophen-4-yl)amino)-1H-pyrazol-5-yl)cyclopentyl isopropylcarbamate A solution of (1R,3S)-3-(1-(tert-butyl)-5-((5-fluoro-1,1-dioxido-2,3-dihydrobenzo [b]thiophen-4-yl)amino)-1H-pyrazol-3-yl)cyclopentyl isopropylcarbamate (90 mg, 0.183 mmol) in HCOOH (3 mL) and H₂O (1 mL) was stirred 100° C. overnight. The reaction mixture was concentrated. The residue was purified by prep-HPLC to afford the title compound (1R,3S)-3-{15-[(5-fluoro-1,1-dioxo-2,3-dihydro-1λ6-benzo[b]thiophen-4-yl)amino]-2H-pyrazol-3-yl}cyclopentyl (prop-2-ylamino)methanoate (14.5 mg, 0.033 mmol, 18.1%) as a white solid. The residue was purified by prep-HPLC to afford the title compound (1R,3S)-3-(3-((5-fluoro-1,1-dioxido-2,3-dihydrobenzo[b]thiophen-4-yl)amino)-1H-pyrazol-5-yl)cyclopentyl isopropylcarbamate (15 mg, 0.033 mmol, 18%) as a white solid. LCMS: ESI m/z 437 [M+H]⁺. ¹H NMR (400 MHz, DMSO) δ 11.72 (s, 1H), 7.87 (s, 1H), 7.42-7.31 (m, 2H), 6.93 (d, J=7.6 Hz, 1H), 5.56 (s, 1H), 4.98 (s, 1H), 3.64-3.49 (m, 3H), 3.15-2.96 (m, 3H), 2.52-2.46 (m, 1H), 2.12-1.86 (m, 2H), 1.79-1.54 (m, 3H), 1.02 (d, J=6.4 Hz, 6H).

### Example 53 Synthesis of (1R,3S)-3-(3-((2,2-dioxido-3,4-dihydro-1H-benzo[c][1,2]thiazin-5-yl)amino)-1H-pyrazol-5-yl)cyclopentyl isopropylcarbamate

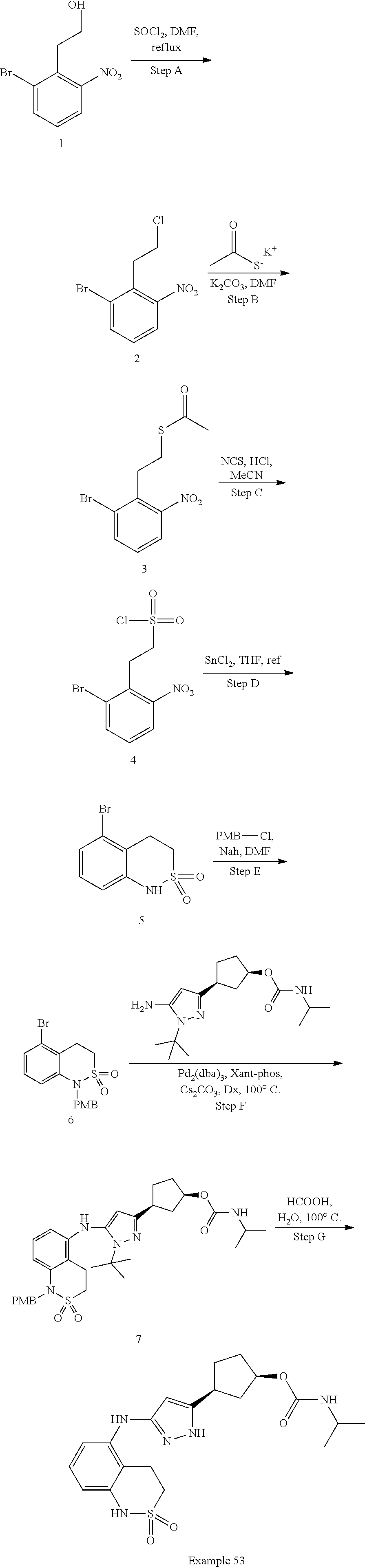

Step A. 1-bromo-2-(2-chloroethyl)-3-nitrobenzene. To a stirred solution of 2-(6-bromo-2-nitrophenyl)ethan-1-ol (500 mg, 2.1 mmol) in SOCl2 (5 mL, 69 mmol) was added DMF (0.1 mL, 1.3 mmol) at rt. After stirring at 100° C. for 2 h, the cooled mixture was concentrated to give crude 1-bromo-2-(2-chloroethyl)-3-nitrobenzene (480 mg, 1.8 mmol, 89%) as a yellow oil. No MS.

Step B. S-[2-(6-bromo-2-nitrophenyl)ethyl] ethanethioate. To a stirred solution of 1-bromo-2-(2-chloroethyl)-3-nitrobenzene (430 mg, 1.6 mmol) in DMF (2 mL) was added potassium acetylsulfanide (371 mg, 3.2 mmol) and K₂CO₃ (449 mg, 3.2 mmol) at rt. After stirring at 50° C. for 2 h, the cooled mixture was poured into water (10 mL) and extracted with EtOAc (2×10 mL). The combined organic phase was washed with brine, dried with Na₂SO₄, filtered and concentrated. The residue was purified by chromatography (silica gel, 0-30%, EtOAc in PE) to give S-[2-(6-bromo-2-nitrophenyl)ethyl] ethanethioate (390 mg, 1.3 mmol, 78%) as a brown oil. LCMS: m/z 304/306 [M+H]⁺.

Step C. 2-(6-bromo-2-nitrophenyl)ethane-1-sulfonyl chloride. To a stirred solution of S-[2-(6-bromo-2-nitrophenyl)ethyl] ethanethioate (390 mg, 1.3 mmol) in MeCN (6 mL) was added HCl (6.4 mL, 12.8 mmol, 2N) and NCS (513 mg, 3.8 mmol) at 0° C. After stirring at 0° C. for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-30%, EtOAc in PE) to give 2-(6-bromo-2-nitrophenyl)ethane-1-sulfonyl chloride (300 mg, 0.91 mmol, 71%) as a yellow oil. LCMS: m/z 308/310 [M+H]⁻.

Step D. 5-bromo-3,4-dihydro-1H-benzo[c][1,2]thiazine 2,2-dioxide. To a stirred solution of 2-(6-bromo-2-nitrophenyl)ethane-1-sulfonyl chloride (200 mg, 0.61 mmol) in THF (10 mL) was added SnCl₂ (413 mg, 1.8 mmol) at rt. After stirred at 80° C. for 3 h, the cooled mixture was filtered. The filtrate was poured into water (10 mL) and extracted with EtOAc (10 mL*2). The combined organic phase was washed with brine, dried with Na₂SO₄, filtered and concentrated. The residue was purified by chromatography (silica gel, 0-40%, EtOAc in PE) to give 5-bromo-3,4-dihydro-1H-benzo[c][1,2]thiazine 2,2-dioxide (100 mg, 0.38 mmol, 63%) as a white solid. LCMS: m/z 262 [M+H]⁺.

Step E. 5-bromo-1-(4-methoxybenzyl)-3,4-dihydro-1H-benzo[c][1,2]thiazine 2,2-dioxide. To a stirred solution of 5-bromo-3,4-dihydro-1H-benzo[c][1,2]thiazine 2,2-dioxide (100 mg, 0.38 mmol) in DMF (3 mL) was added NaH (46 mg, 1.1 mmol, 60% in mineral oil) and PMB-Cl (0.1 mL, 0.76 mmol) at 0° C. After stirred at rt for 2 h, the mixture was poured into ice-water (10 mL) and extracted with EtOAc (10 mL*2). The combined organic phase was washed by brine, dried over Na₂SO₄, filtered and concentrated. The residue was purified by prep-TLC (PE: EtOAc=3:1) to give 5-bromo-1-(4-methoxybenzyl)-3,4-dihydro-1H-benzo[c][1,2]thiazine 2,2-dioxide (80 mg, 0.21 mmol, 55%) as a yellow oil. No MS.

Step F. (1R,3S)-3-(1-(tert-butyl)-5-((1-(4-methoxybenzyl)-2,2-dioxido-3,4-dihydro-1H-benzo[c][1,2]thiazin-5-yl)amino)-1H-pyrazol-3-yl)cyclopentyl isopropylcarbamate. To a stirred solution of 5-bromo-1-(4-methoxybenzyl)-3,4-dihydro-1H-benzo[c][1,2]thiazine 2,2-dioxide (80 mg, 0.21 mmol) in dioxane (8 mL) was added (1R,3S)-3-[5-amino-1-(2-methylprop-2-yl)pyrazol-3-yl]cyclopentyl (prop-2-ylamino)methanoate (64 mg, 0.21 mmol), Xant-PHOS (24 mg, 0.04 mmol), Pd₂(dba)₃ (19 mg, 0.02 mmol) and Cs₂CO₃ (204 mg, 0.63 mmol) at rt. After stirred at 100° C. under N₂ for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-70%, EtOAc in PE) to give crude (1R,3S)-3-(1-(tert-butyl)-5-((1-(4-methoxybenzyl)-2,2-dioxido-3,4-dihydro-1H-benzo[c][1,2]thiazin-5-yl)amino)-1H-pyrazol-3-yl)cyclopentyl isopropylcarbamate (60 mg, 0.06 mmol, 28%) as a yellow oil. LCMS: m/z 610 [M+H]⁺.

Step G. (1R,3S)-3-(3-((2,2-dioxido-3,4-dihydro-1H-benzo[c][1,2]thiazin-5-yl)amino)-1H-pyrazol-5-yl)cyclopentyl isopropylcarbamate. To a stirred solution of (1R,3S)-3-(1-(tert-butyl)-5-((1-(4-methoxybenzyl)-2,2-dioxido-3,4-dihydro-1H-benzo[c][1,2]thiazin-5-yl)amino)-1H-pyrazol-3-yl)cyclopentyl isopropylcarbamate (60 mg, 0.06 mmol) in HCOOH (3 mL) was added H₂O (0.3 mL, 16 mmol) at rt. After stirred at 100° C. for 5 h, the cooled mixture was concentrated. The residue was purified by chromatography (silica gel, 0-70%, EtOAc in PE) and then prep-HPLC (C18, 40˜ 90% MeCN in H₂O with 0.1% HCOOH) to give (1R,3S)-3-(3-((2,2-dioxido-3,4-dihydro-1H-benzo[c][1,2]thiazin-5-yl)amino)-1H-pyrazol-5-yl)cyclopentyl isopropylcarbamate (3 mg, 0.007 mmol, 7%) as a white solid. LCMS: m/z 434 [M+H]⁺. ¹H NMR (400 MHz, MeOD) δ 7.20 (dd, J=8.0, 8.0 Hz, 1H), 6.98 (d, J=8.0 Hz, 1H), 6.62 (d, J=8.0 Hz, 1H), 5.08 (s, 1H), 3.66-3.52 (m, 1H), 3.21-3.12 (m, 1H), 2.60-2.50 (m, 1H), 2.19-2.10 (m, 1H), 1.99-1.76 (m, 4H), 1.18-1.08 (m, 6H).

### Example 54: (1R,3S)-3-(3-((6-fluoro-1,1-dioxido-2,3-dihydrobenzo[b]thiophen-5-yl)amino)-1H-pyrazol-5-yl)cyclopentyl isopropylcarbamate

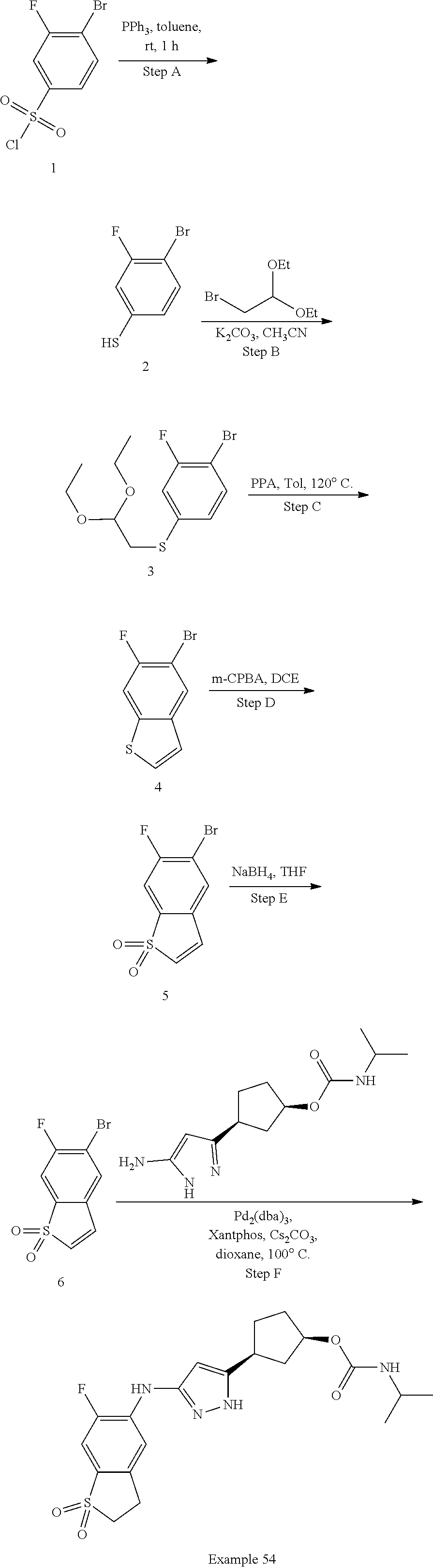

Step A: 4-bromo-3-fluorobenzenethiol To a solution of 4-bromo-3-fluorobenzenesulfonyl chloride (5 g, 18.2 mmol) in toluene (100 mL) was added PPh₃ (19.1 g, 73.1 mmol). The reaction mixture was stirred at 50° C. for 30 min. The cooled reaction mixture was diluted with H₂O, extracted with EA. The organic phase was mixed with 10% NaOH aqueous solution, the aqueous layer washed with EA, acidified with HCl, extracted with DCM. The organic phase was dried by Na₂SO₄ and concentrated. The residue was purified using silica gel column chromatography eluting with EA in PE [Gradient: 0-30%] afford the title compound 4-bromo-3-fluorobenzene-1-thiol (2.4 g, 11.5 mmol, 63.4%) as a colorless oil. No MS.

Step B: (4-bromo-3-fluorophenyl)(2,2-diethoxyethyl)sulfane To a solution of 4-bromo-3-fluorobenzene-1-thiol (2.4 g, 11.5 mmol) in CH₃CN (30 mL) were added K₂CO₃ (3.20 g, 23.1 mmol) and 2-bromo-1,1-diethoxyethane (2.67 mL, 17.3 mmol). The reaction mixture was stirred at 60° C. for 3 h. The reaction mixture was diluted with H₂O, extracted with EA, dried by Na₂SO₄ and concentrated. The residue was purified using silica gel column chromatography eluting with EA in PE [Gradient: 0-35%] afford the title compound 2-[(4-bromo-3-fluorophenyl)sulfanyl]-1,1-diethoxyethane (1.5 g, 4.64 mmol, 40.0%) as a colorless oil. No MS. ¹H NMR (400 MHz, CDCl₃) δ 7.65 (d, J=8.4 Hz, 1H), 7.33-7.24 (m, 2H), 3.47-3.37 (m, 2H), 3.07-2.97 (m, 2H), 2.53-2.41 (m, 2H), 1.38-1.21 (m, 6H).

Step C: 5-bromo-6-fluorobenzo[b]thiophene To a solution of 2-[(4-bromo-3-fluorophenyl)sulfanyl]-1,1-diethoxyethane (1.5 g, 4.64 mmol) in toluene (30 mL) was added polyphosphoric acid (PPA, 1.14 g, 13.9 mmol). The reaction mixture was stirred at 120° C. for 12 h. The reaction mixture was diluted with H₂O, extracted with EA, dried by Na₂SO₄ and concentrated. The residue was purified using silica gel column chromatography eluting with EA in PE [Gradient: 0-35%] afford the title compound 5-bromo-6-fluorobenzothiophene (0.5 g, 2.16 mmol, 46.6%) as a colorless oil. No MS. ¹H NMR (400 MHz, CDCl3) δ 7.60 (d, J=5.6 Hz, 1H), 7.49 (d, J=6.0 Hz, 1H), 7.18 (d, J=6.8 Hz, 1H), 6.78 (d, J=6.8 Hz, 1H).

Step D: 5-bromo-6-fluorobenzo[b]thiophene 1,1-dioxide 1,1-dioxide. To a solution of 5-bromo-6-fluorobenzothiophene (500 mg, 2.16 mmol) in DCE (8 mL) was added 3-chlorobenzene-1-carboperoxoic acid (747 mg, 4.32 mmol). The reaction mixture was stirred at 60° C. overnight. The cooled reaction mixture was quenched with Na₂S₂O₃, extracted with EA, dried by Na₂SO₄ and concentrated. The residue was purified using silica gel column chromatography eluting with EA in PE [Gradient: 0-40%] afford the title compound 5-bromo-6-fluorobenzo[b]thiophene 1,1-dioxide (200 mg, 0.760 mmol, 35%) as a white solid. No MS.

Step E: 5-bromo-6-fluoro-2,3-dihydrobenzo[b]thiophene 1,1-dioxide To a solution of 5-bromo-6-fluorobenzo[b]thiophene 1,1-dioxide (200 mg, 0.760 mmol) in THF (5 mL) was added NaBH₄ (26 mg, 0.760 mmol) at 0° C. The reaction mixture was stirred at 0° C. for 3 h. The reaction mixture was quenched with NH₄Cl, extracted with EA, dried by Na₂SO₄ and concentrated. The residue was purified using silica gel column chromatography eluting with EA in PE [Gradient: 0-50%] afford the title compound 5-bromo-6-fluoro-2,3-dihydrobenzo[b]thiophene 1,1-dioxide (140 mg, 0.528 mmol, 69%) as a white solid. No LCMS.

Step F: (1R,3S)-3-(3-((6-fluoro-1,1-dioxido-2,3-dihydrobenzo[b]thiophen-5-yl)amino)-1H-pyrazol-5-yl)cyclopentyl isopropylcarbamate To a solution of (1R,3S)-3-(5-amino-1H-pyrazol-3-yl)cyclopentyl (prop-2-ylamino)methanoate (140 mg, 0.555 mmol) in dioxane (5 mL) were added 5-bromo-6-fluoro-2,3-dihydrobenzo[b]thiophene 1,1-dioxide (147. mg, 0.555 mmol), Cs₂CO₃ (361 mg, 1.110 mmol), Xant-PHOS (64.2 mg, 0.11 mmol) and Pd₂(dba)₃ (50.8 mg, 0.055 mmol). The reaction mixture was stirred at 100° C. for 12 h. The reaction mixture was diluted with H₂O, extracted with EA, dried by Na₂SO₄ and concentrated. The residue was purified by prep-HPLC to afford the title compound (1R,3S)-3-{5-[(6-fluoro-1,1-dioxo-2,3-dihydro-1λ⁶-benzothiophen-5-yl)amino]-1H-pyrazol-3-yl}cyclopentyl (prop-2-ylamino)methanoate (37 mg, 0.085 mmol, 15%) as a white solid. LCMS: ESI m/z 437 [M+H]⁺. ¹H NMR (400 MHz, DMSO) δ 8.74 (s, 1H), 8.11 (s, 1H), 7.54 (d, J=10.0 Hz, 1H), 6.93 (s, 1H), 5.85 (s, 1H), 4.99 (s, 1H), 3.58-3.49 (m, 3H), 3.29-3.21 (m, 2H), 3.09-3.02 (m, 1H), 2.48-2.39 (m, 1H), 2.06-1.88 (m, 2H), 1.78-1.55 (m, 3H), 1.03 (d, J=6.0 Hz, 6H)

### Example 55: Synthesis of (1R,3S)-3-(3-((1,1-dioxido-2,3-dihydrothieno[3,2-b]pyridin-5-yl)amino)-1H-pyrazol-5-yl)cyclopentyl isopropylcarbamate

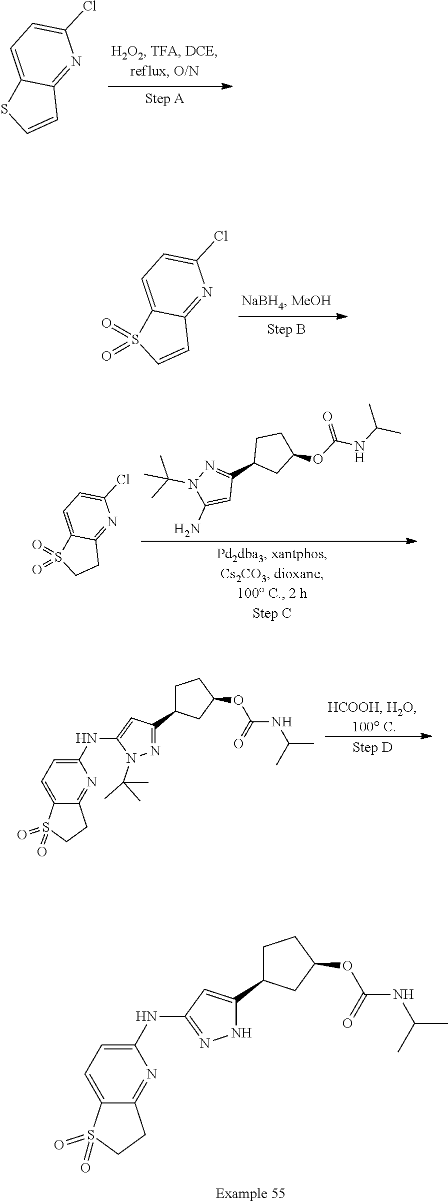

Step A. 5-chloro-1λ<sup>6</sup>-thieno[3,2-b]pyridine-1,1-dione. To a stirred solution of 5-chlorothieno[3,2-b]pyridine (5 g, 29.5 mmol) in DCE (25 mL) was added TFA (10 mL) and H₂O₂(10 mL, 97.9 mmol, 30% in H₂O) slowly at rt. After stirred at 90° C. overnight, the cooled mixture was quenched with NaS₂O₃ (aq, 20 mL) and extracted with DCM (10 mL*2). The combined organic phase was washed with brine, dried with Na₂SO₄, filtered and concentrated. The residue was purified by chromatography (silica gel, 0-70%, EtOAc in PE) to give 5-chloro-1λ⁶-thieno[3,2-b]pyridine-1,1-dione (1.9 g, 9.4 mmol, 32%) as a white solid. LCMS: m/z 202 [M+H]⁺.

Step B. 5-chloro-2,3-dihydro-1λ<sup>6</sup>-thieno[3,2-b]pyridine-1,1-dione. To a stirred solution of 5-chloro-1)⁶-thieno[3,2-b]pyridine-1,1-dione (500 mg, 2.5 mmol) in MeOH (3 mL)/THF (5 mL) was added NaBH₄ (168 mg, 4.9 mmol) slowly at 0° C. After stirring at rt for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-40%, EtOAc in PE) to give 5-chloro-2,3-dihydro-1⁶-thieno[3,2-b]pyridine-1,1-dione (260 mg, 1.3 mmol, 51%) as a white solid. LCMS: m/z 204 [M+H]⁺. ¹H NMR (400 MHz, DMSO) δ 8.37 (d, J=8.4 Hz, 1H), 7.69 (d, J=8.4 Hz, 1H), 3.77 (t, J=6.8 Hz, 2H), 3.47 (t, J=6.8 Hz, 2H).

Step C. (1R,3S)-3-{5-[(1,1-dioxo-2,3-dihydro-1λ<sup>6</sup>-thieno[3,2-b]pyridin-5-yl)amino]-1-(2-methylprop-2-yl)pyrazol-3-yl}cyclopentyl (prop-2-ylamino)methanoate. To a stirred solution of 5-chloro-2,3-dihydro-1λ⁶-thieno[3,2-b]pyridine-1,1-dione (100 mg, 0.49 mmol) in dioxane (8 mL) was added (1R,3S)-3-[5-amino-1-(2-methylprop-2-yl)pyrazol-3-yl]cyclopentyl (prop-2-ylamino)methanoate (90 mg, 0.29 mmol), Xant-PHOS (34 mg, 0.06 mmol), Pd₂(dba)₃ (27 mg, 0.03 mmol) and Cs₂CO₃ (238 mg, 0.73 mmol) at rt. After stirring at 100° C. under N₂ for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-80%, EtOAc in PE) to give crude (1R,3S)-3-{5-[(1,1-dioxo-2,3-dihydro-1λ⁶-thieno[3,2-b]pyridin-5-yl)amino]-1-(2-methylprop-2-yl)pyrazol-3-yl}cyclopentyl (prop-2-ylamino)methanoate (140 mg, 0.18 mmol, 60%) as a yellow solid. LCMS: m/z 476 [M+H]⁺.

Step D. (1R,3S)-3-(3-((1,1-dioxido-2,3-dihydrothieno[3,2-b]pyridin-5-yl)amino)-1H-pyrazol-5-yl)cyclopentyl isopropylcarbamate. To a stirred solution of (1R,3S)-3-(1-(tert-butyl)-5-((1,1-dioxido-2,3-dihydrothieno[3,2-b]pyridin-5-yl)amino)-1H-pyrazol-3-yl)cyclopentyl isopropylcarbamate (140 mg, 0.29 mmol) in HCOOH (6 mL) was added H₂O (1 mL, 55 mmol) at rt. After stirred at 100° C. for 5 h, the cooled mixture was concentrated. The residue was purified by chromatography (silica gel, 0-6%, MeOH in DCM) and then prep-HPLC (C18, 40˜ 90% MeCN in H₂O with 0.1% TFA) to give (1R,3S)-3-(3-((1,1-dioxido-2,3-dihydrothieno[3,2-b]pyridin-5-yl)amino)-1H-pyrazol-5-yl)cyclopentyl isopropylcarbamate (58 mg, 0.14 mmol, 47%) as a white solid. LCMS: m/z 420 [M+H]⁺. ¹H NMR (400 MHz, DMSO) δ 10.14 (s, 1H), 7.90 (d, J=8.8 Hz, 1H), 7.22 (d, J=8.8 Hz, 1H), 6.95 (d, J=7.2 Hz, 1H), 6.23 (s, 1H), 5.01 (s, 1H), 3.69-3.52 (m, 3H), 3.30 (t, J=6.8 Hz, 2H), 3.15-3.02 (m, 1H), 2.50-2.42 (m, 1H), 2.07-1.98 (m, 1H), 1.97-1.88 (m, 1H), 1.85-1.76 (m, 2H), 1.7-1.66 (m, 1H), 1.03 (d, J=6.0 Hz, 6H).

### Example 56: (1R,3S)-3-{5-[(2-methyl-1,1-dioxo-3,4-dihydro-2H-1λ6-benzo[2,1-e][1,2]thiazin-5-yl)amino]-1H-pyrazol-3-yl}cyclopentyl (prop-2-ylamino)methanoate

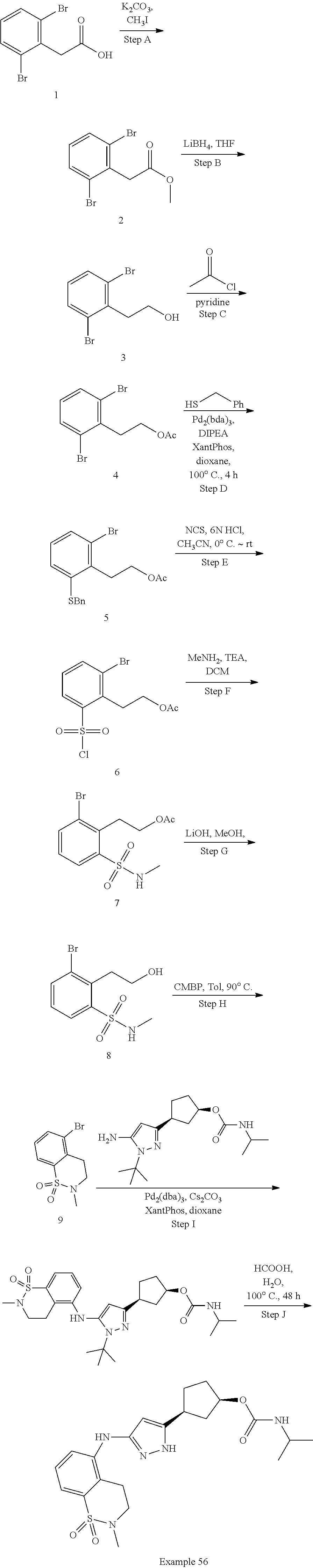

Step A: methyl 2-(2,6-dibromophenyl)acetate To a solution of (2,6-dibromophenyl)acetic acid (3 g, 10.2 mmol) in DMF (5 mL) were added K₂CO₃ (2.82 g, 20.4 mmol), CH₃I (0.953 mL, 15.3 mmol). The reaction mixture was stirred at rt under N₂ overnight. The reaction mixture was diluted with H₂O, extracted with EA, washed with brine, dried by Na₂SO₄, filtered and concentrated. The residue was purified using silica gel column chromatography eluting with EA in PE [Gradient: 0-7%] to afford the title compound methyl (2,6-dibromophenyl)acetate (2.9 g, 9.41 mmol, 92.2%) as a white solid. No MS. ¹H NMR (400 MHz, CDCl3) δ 7.55 (d, J=8.0 Hz, 2H), 7.01 (t, J=8.0 Hz, 1H), 4.12 (s, 2H), 3.73 (s, 3H).

Step B: 2-(2,6-dibromophenyl)ethan-1-ol To a solution of methyl (2,6-dibromophenyl)acetate (600 mg, 1.94 mmol) in THF (10 mL) was added LiBH₄ (1.9 mL, 1.94 mmol, 1M in THF). The reaction mixture was stirred at 60° C. overnight. The cooled reaction mixture was diluted with H₂O, extracted with EA, washed with brine, dried by Na₂SO₄ and concentrated. The residue was purified using silica gel column chromatography eluting with EA in PE [Gradient: 0-30%] afford the title compound 2-(2,6-dibromophenyl)ethan-1-ol (430 mg, 1.53 mmol, 78.8%) as a colorless oil. No MS. ¹H NMR (400 MHz, CDCl₃) δ 7.52 (d, J=8.0 Hz, 2H), 6.94 (t, J=8.0 Hz, 1H), 3.88 (t, J=7.2 Hz, 2H), 3.33 (t, J=7.2 Hz, 2H).

Step C: 2,6-dibromophenethyl acetate To a solution of 2-(2,6-dibromophenyl)ethan-1-ol (430 mg, 1.53 mmol) in DCM (5 mL) were added DIEA (0.76 mL, 4.60 mmol) and acetyl chloride (0.22 mL, 3.07 mmol). The reaction mixture was stirred at 0° C. for 3 h. The reaction mixture was concentrated. The residue was purified using silica gel column chromatography eluting with EA in PE [Gradient: 0-40%] afford the title compound 2-(2,6-dibromophenyl)ethyl acetate (340 mg, 1.05 mmol, 69%) as a yellow solid. No MS. ¹H NMR (400 MHz, CDCl₃) δ 7.52 (d, J=8.0 Hz, 2H), 6.96 (t, J=8.0 Hz, 1H), 4.31 (t, J=7.2 Hz, 2H), 3.37 (t, J=7.2 Hz, 2H), 2.06 (s, 3H).

Step D: 2-(benzylthio)-6-bromophenethyl acetate To a solution of 2-(2,6-dibromophenyl)ethyl acetate (340 mg, 1.55 mmol) in dioxane (5 mL) were added phenylmethanethiol (0.18 mL, 1.56 mmol), ethyl[di(prop-2-yl)]amine (0.513 mL, 3.10 mmol), Xant-PHOS (179 mg, 0.311 mmol) and Pd₂(dba)₃ (142 mg, 0.155 mmol). The reaction mixture was stirred at 100° C. under N₂ atmosphere for 4 h. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 10%] to afford the title compound 2-[6-(benzylsulfanyl)-2-bromophenyl]ethyl acetate (270 mg, 0.74 mmol, 47%) as a colorless oil. ¹H NMR (400 MHz, DMSO) δ 7.48-7.44 (m, 2H), 7.38-7.23 (m, 6H), 7.16 (t, J=8.0 Hz, 1H), 4.27 (s, 2H), 4.12 (t, J=7.2 Hz, 2H), 3.16 (t, J=7.2 Hz, 2H), 1.98 (s, 3H).

Step E: 2-bromo-6-(chlorosulfonyl)phenethyl acetate To a stirred suspension of 2-[6-(benzylsulfanyl)-2-bromophenyl]ethyl acetate (1.2 g, 3.28 mmol) in CH₃CN (10 mL) and 6N HCl (10 mL) was added NCS (2.19 g, 16.4 mmol) at 0° C. The reaction mixture was allowed to warm to ambient temperature and stirred for 2 hours. The reaction was partitioned between EtOAc and water. The aqueous layer was extracted with EtOAc. The combined organic layers were washed with water and brine, dried and concentrated to give a crude desire product 2-bromo-6-(chlorosulfonyl)phenethyl acetate (400 mg crude) as a light yellow oil which was used directly for next step without purification.

Step F: 2-{2-bromo-6-[(methylamino)dioxo-%6-sulfanyl]phenyl}ethyl acetate The above 2-bromo-6-(chlorosulfonyl)phenethyl acetate was dissolved in DCM (10 mL) was added into a solution of MeNH₂ in THF (10 Ml, 2M in THF) at 0° C. The reaction mixture was allowed to warm to ambient temperature and stirred for 1 h. The reaction was partitioned between EtOAc and water. The aqueous layer was extracted with EtOAc. The combined organic layer was washed with water and brine, dried over Na₂SO₄. Filtered and concentrated. The residue was purified by a silica column eluted with PE: EA (gradient: 0˜ 50%) to give the desire product 2-{2-bromo-6-[(methylamino)dioxo-λ⁶-sulfanyl]phenyl}ethyl acetate (200 mg, 1.19 mmol, 36%, 2 steps) as a colorless oil. LCMS m/z: 335 [M+H]⁺

Step G: 3-bromo-2-(2-hydroxyethyl)-N-methylbenzenesulfonamide A solution of 2-{2-bromo-6-[(methylamino)dioxo-λ⁶-sulfanyl]phenyl}ethyl acetate (200 mg, 0.6 mmol) and LiOH (83 mg, 1.98 mmol) in MeOH (3 mL) and H₂O (3 mL) was a stirred at room temperature for 2 hours. The reaction was partitioned between EtOAc and water. The aqueous layer was extracted with EtOAc. The combined organic layers were washed with water and brine, dried and concentrated. The residue was purified by silica gel column chromatography eluted with ethyl acetate in petroleum ether (gradient: 0˜ 40%) to afford the title compound 3-bromo-2-(2-hydroxyethyl)-N-methylbenzenesulfonamide (140 mg, 0.45 mmol, 80%) as a colorless oil. LCMS: m/z 295 [M+H]⁺. ¹H NMR (400 MHz, DMSO) δ 7.90 (d, J=8.0 Hz, 1H), 7.82 (d, J=8.0 Hz, 1H), 7.37 (t, J=8.0 Hz, 2H), 4.98 (s, 1H), 3.61 (t, J=7.6 Hz, 2H), 3.35-3.25 (m, 2H), 2.49 (s, 3H).

Step H: 5-bromo-2-methyl-3,4-dihydro-2H-1λ6-benzo[2,1-e][1,2]thiazine-1,1-dione A solution of 3-bromo-2-(2-hydroxyethyl)-N-methylbenzenesulfonamide (140 mg, 0.48 mmol) and 2-(tributylphosphoranylidene)acetonitrile (172 mg, 0.71 mmol) in toluene (5 mL) was stirred at 100° C. under N₂ atmosphere for 2 h. TLC and LCMS indicated the reaction was over. The cooled mixture was concentrated and the residue was purified by silica gel column chromatography eluted with ethyl acetate in petroleum ether. (gradient: 0˜ 30%) to afford the title compound 5-bromo-2-methyl-3,4-dihydro-2H-6-benzo[2,1-e][1,2]thiazine-1,1-dione (70 mg, 0.25 mmol, 53%) as an off-white solid. LCMS: m/z 276 [M+H]⁺.

Step I: (1R,3S)-3-[1-(2-methylprop-2-yl)-5-{[2-(2-methylprop-2-yl)-1,1-dioxo-3,4-dihydro-2H-1λ6-benzo[2,1-e][1,2]thiazin-5-yl]amino}pyrazol-3-yl]cyclopentyl (prop-2-ylamino)methanoate To a solution of 5-bromo-2-methyl-3,4-dihydro-2H-1λ⁶-benzo[2,1-e][1,2]thiazine-1,1-dione (70 mg, 0.25 mmol), (1R,3S)-3-[5-amino-1-(2-methylprop-2-yl)pyrazol-3-yl]cyclopentyl (prop-2-ylamino)methanoate (78 mg, 0.25 mmol), Xantphos (15 mg, 0.025 mmol) and Cs₂CO₃ (165 mg, 0.50 mmol) in 1,4-dioxane (8 mL) was added Pd₂(dba)₃ (23 mg, 0.025 mmol). The reaction was stirred at 100° C. under N₂ atmosphere for 4 h. TLC and LCMS indicted the reaction was over. The reaction was poured into ice-water, extracted with EA (20 mL*3), the combined organic layer was washed with brine, dried over Na₂SO₄, filtered and concentrated. The residue was purified by a silica column eluted with EA in PE (gradient: 0-50%) to give the desire product (1R,3S)-3-[1-(2-methylprop-2-yl)-5-{[2-(2-methylprop-2-yl)-1,1-dioxo-3,4-dihydro-2H-1λ⁶-benzo[2,1-e][1,2]thiazin-5-yl]amino}pyrazol-3-yl]cyclopentyl (prop-2-ylamino)methanoate (50 mg, 0.09 mmol, 42%) as a yellow solid. LCMS m/z:504 [M+H]⁺.

Step J: (1R,3S)-3-{5-[(2-methyl-1,1-dioxo-3,4-dihydro-2H-1λ6-benzo[2,1-e][1,2]thiazin-5-yl)amino]-1H-pyrazol-3-yl}cyclopentyl (prop-2-ylamino)methanoate. A solution of (1R,3S)-3-{5-[(2-methyl-1,1-dioxo-3,4-dihydro-2H-1λ6-benzo[2,1-e][1,2]thiazin-5-yl)amino]-1-(2-methylprop-2-yl)pyrazol-3-yl}cyclopentyl (prop-2-ylamino)methanoate (50 mg, 0.099 mmol) in HCOOH (3 mL) and H₂O (0.3 mL) was stirred at 100° C. for 48 h. The reaction was concentrated and the residue was purified by prep-HPLC to afford the title compound (1R,3S)-3-{5-[(2-methyl-1,1-dioxo-3,4-dihydro-2H-1λ6-benzo[2,1-e][1,2]thiazin-5-yl)amino]-1H-pyrazol-3-yl}cyclopentyl (prop-2-ylamino)methanoate (14 mg, 0.031 mmol, 32%) as a white solid. LCMS: 448 [M+H]⁺. ¹H NMR (400 MHz, DMSO) δ 7.83 (d, J=8.0 Hz, 1H), 7.63 (s, 1H), 7.30 (t, J=8.0 Hz, 1H), 7.21-7.15 (m, 1H), 6.94 (d, J=6.8 Hz, 1H), 5.82 (s, 1H), 5.00 (s, 1H), 3.87 (t, J=6.0 Hz, 2H), 3.14-2.95 (m, 1H), 2.79 (t, J=6.0 Hz, 2H), 2.76 (s, 3H), 2.49-2.43 (m, 1H), 2.07-1.97 (m, 1H), 1.96-1.85 (m, 1H), 1.79-1.56 (m, 3H), 1.03 (d, J=5.6 Hz, 6H).

### Example 57: Synthesis of (1R,3S)-3-{5-[(1,1-dioxo-2,3-dihydro-1λ<sup>6</sup>-thieno[3,2-c]pyridin-4-yl)amino]-2H-pyrazol-3-yl}cyclopentyl (prop-2-ylamino)methanoate

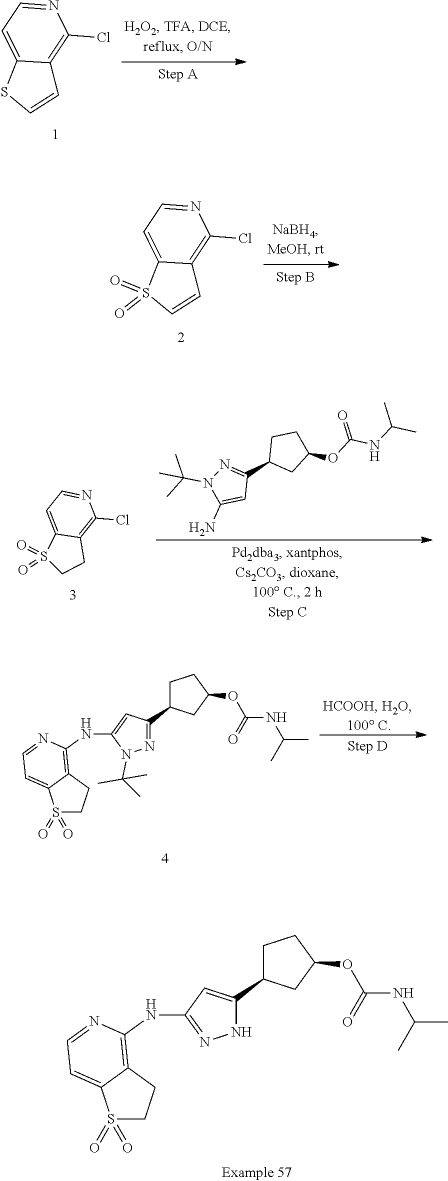

Step A. 4-chlorothieno[3,2-c]pyridine 1,1-dioxide. To a stirred solution of 4-chlorothieno[3,2-c]pyridine (5 g, 29.5 mmol) in DCE (30 mL) was added TFA (10 mL) and H₂O₂(15 mL, 146.9 mmol, 30% in H₂O) slowly at rt. After stirring at 90° C. overnight, the cooled mixture was quenched with NaS₂O₃ (aq, 20 mL) and extracted with DCM (10 mL*2). The combined organic phase was washed with brine, dried with Na₂SO₄, filtered and concentrated. The residue was purified by chromatography (silica gel, 0-70%, EtOAc in PE) to 4-chlorothieno[3,2-c]pyridine 1,1-dioxide (380 mg, 1.9 mmol, 6.4%) as a brown solid. LCMS: m/z 202 [M+H]⁺.

Step B. 4-chloro-2,3-dihydro-1λ<sup>6</sup>-thieno[3,2-c]pyridine-1,1-dione. To a stirred solution of 4-chlorothieno[3,2-c]pyridine 1,1-dioxide (380 mg, 1.9 mmol) in MeOH (5 mL)/THF (5 mL) was added NaBH₄ (127 mg, 3.7 mmol) slowly at 0° C. After stirring at rt for 1 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-45%, EtOAc in PE) to give 4-chloro-2,3-dihydro-1λ⁶-thieno[3,2-c]pyridine-1,1-dione (240 mg, 1.2 mmol, 62%) as a white solid. LCMS: m/z 204 [M+H]⁺. Step C. (1R,3S)-3-{5-[(1,1-dioxo-2,3-dihydro-1λ<sup>6</sup>-thieno[3,2-c]pyridin-4-yl)amino]-1-(2-methylprop-2-yl)pyrazol-3-yl}cyclopentyl (prop-2-ylamino)methanoate.

To a stirred solution of 4-chloro-2,3-dihydro-1λ⁶-thieno[3,2-c]pyridine-1,1-dione (100 mg, 0.49 mmol) in dioxane (8 mL) was added (1R,3S)-3-[5-amino-1-(2-methylprop-2-yl)pyrazol-3-yl]cyclopentyl (prop-2-ylamino)methanoate (75 mg, 0.24 mmol), xant-PHOS (33 mg, 0.06 mmol), Pd₂(dba)₃ (27 mg, 0.03 mmol) and Cs₂CO₃ (238 mg, 0.73 mmol) at rt. After stirring at 100° C. for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-80%, EtOAc in PE) to give crude (1R,3S)-3-{5-[(1,1-dioxo-2,3-dihydro-1⁶-thieno[3,2-c]pyridin-4-yl)amino]-1-(2-methylprop-2-yl)pyrazol-3-yl}cyclopentyl (prop-2-ylamino)methanoate (80 mg, 0.101 mmol, 41%) as a yellow solid. LCMS: m/z 476 [M+H]⁺.

Step D. (1R,3S)-3-{5-[(1,1-dioxo-2,3-dihydro-1λ<sup>6</sup>-thieno[3,2-c]pyridin-4-yl)amino]-2H-pyrazol-3-yl}cyclopentyl (prop-2-ylamino)methanoate. To a stirred solution of (1R,3S)-3-{5-[(1,1-dioxo-2,3-dihydro-1λ⁶-thieno[3,2-c]pyridin-4-yl)amino]-1-(2-methylprop-2-yl)pyrazol-3-yl}cyclopentyl (prop-2-ylamino)methanoate (60 mg, 0.13 mmol) in HCOOH (6 mL) was added H₂O (1 mL, 55.494 mmol) at rt. After stirring at 100° C. for 3 h, the cooled mixture was concentrated. The residue was purified by chromatography (silica gel, 0-6%, MeOH in DCM) and then prep-HPLC (C18, 40˜ 90% MeCN in H₂O with 0.1% TFA) to give (1R,3S)-3-{5-[(1,1-dioxo-2,3-dihydro-1λ⁶-thieno[3,2-c]pyridin-4-yl)amino]-2H-pyrazol-3-yl}cyclopentyl (prop-2-ylamino)methanoate (18 mg, 0.043 mmol, 34%) as a white solid. LCMS: m/z 420 [M+H]⁺. ¹H NMR (400 MHz, DMSO) δ 9.62 (s, 1H), 8.31 (d, J=5.2 Hz, 1H), 7.11 (d, J=5.2 Hz, 1H), 6.95 (d, J=6.8 Hz, 1H), 6.36 (s, 1H), 5.01 (s, 1H), 3.57-3.52 (m, 3H), 3.28 (t, J=6.8 Hz, 2H), 3.15-3.07 (m, 1H), 2.49-2.44 (m, 1H), 2.09-2.00 (m, 1H), 1.99-1.89 (m, 1H), 1.77-1.61 (m, 3H), 1.02 (d, J=6.0 Hz, 6H).

### Example 58: (1R,3S)-3-(3-((8-chloro-1,1-dioxidothiochroman-5-yl)amino)-1H-pyrazol-5-yl)cyclopentyl isopropylcarbamate

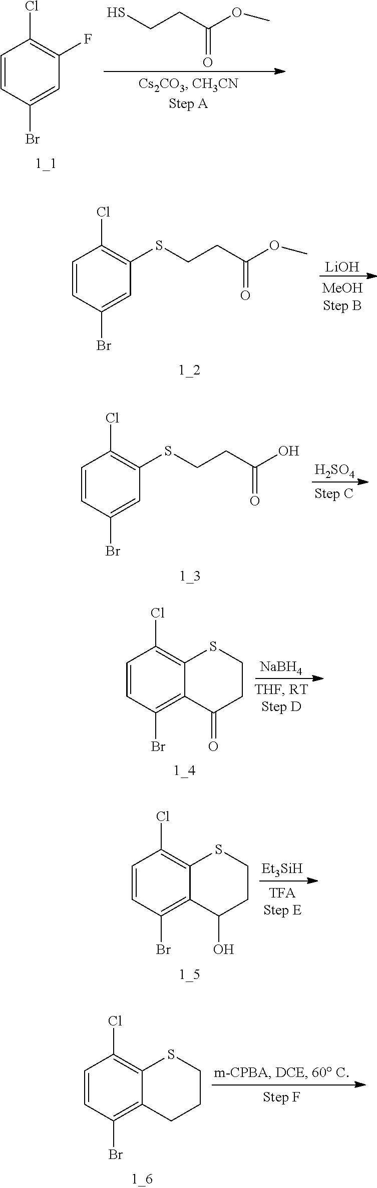

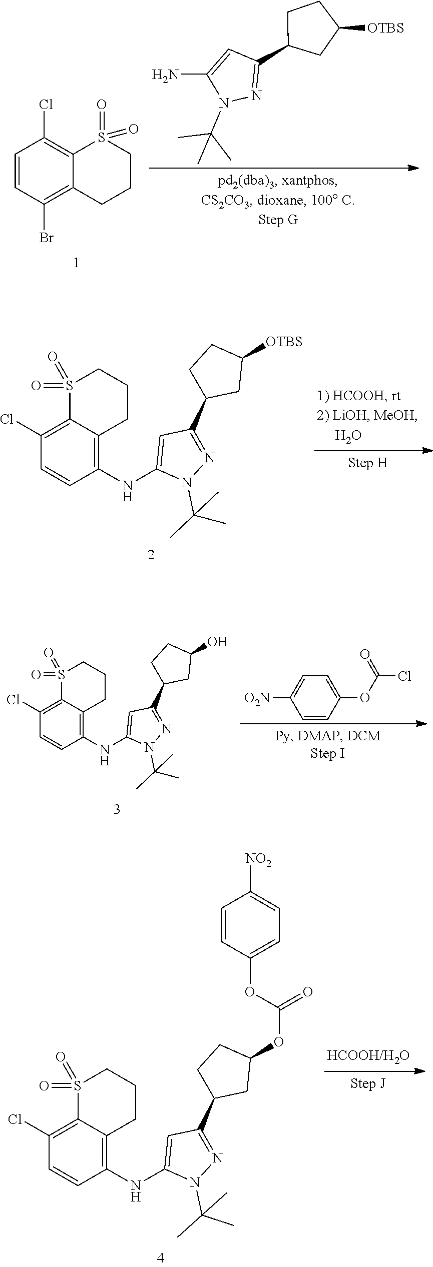

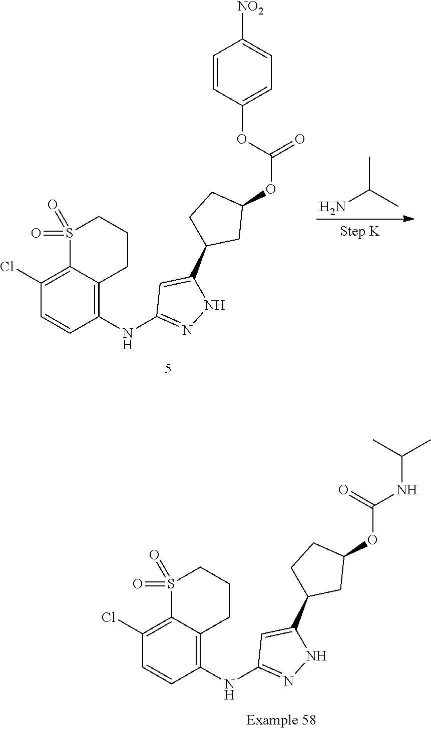

Step A: methyl 3-((5-bromo-2-chlorophenyl)thio)propanoate To a solution of 4-bromo-1-chloro-2-fluorobenzene (17 g, 81.1 mmol) in CH₃CN (20 mL) were added methyl methyl 3-mercaptopropanoate (11.7 g, 97.4 mmol) and Cs₂CO₃ (52.8 g, 162 mmol). The reaction mixture was stirred at RT overnight. The reaction mixture was diluted with H₂O, extracted with EA, washed with brine, dried by Na₂SO₄ and concentrated. The residue was purified using silica gel column chromatography eluting with EA in PE [Gradient: 0-20%] afford the title compound methyl 3-[(5-bromo-2-chlorophenyl)sulfanyl]propanoate (5 g, 16.1 mmol, 20%) as a colorless oil. No MS, ¹H NMR (400 MHz, CDCl3) δ 7.40 (d, J=2.0 Hz, 1H), 7.25-7.20 (m, 2H), 3.72 (s, 3H), 3.21 (t, J=7.2 Hz, 2H), 2.70 (t, J=7.2 Hz, 2H).

Step B: 3-((5-bromo-2-chlorophenyl)thio)propanoic acid To a solution of methyl 3-[(5-bromo-2-chlorophenyl)sulfanyl]propanoate (6 g, 19.3 mmol) in MeOH (60 mL) was added LiOH (1.63 g, 38.7 mmol). The reaction mixture was stirred at RT for 3 h. The reaction mixture was diluted with H₂O, adjusted pH to 3-4, extracted with EA, washed with brine, dried by Na₂SO₄ and concentrated to afford the title compound 3-[(5-bromo-2-chlorophenyl)sulfanyl]propanoic acid (4 g, 13.5 mmol, 69.8%) as a white solid. LCMS: ESI m/z 293 [M+H]⁻

Step C: 5-bromo-8-chlorothiochroman-4-one A solution of 3-[(5-bromo-2-chlorophenyl)sulfanyl]propanoic acid (4 g, 13.5 mmol) in H₂SO₄ (40 mL) was stirred at RT overnight. The reaction mixture was poured in ice water, extracted with EA, washed with brine, dried by Na₂SO₄ and concentrated. The residue was purified using silica gel column chromatography eluting with EA in PE [Gradient: 0-25%] afford the title compound 5-bromo-8-chloro-3,4-dihydro-2H-1-benzothiin-4-one (600 mg, 2.16 mmol, 15.9%) as a colorless oil. No MS, ¹H NMR (400 MHz, CDCl3) δ 7.39 (d, J=8.4 Hz, 1H), 7.28-7.24 (m, 1H), 3.31-3.23 (m, 2H), 3.08-3.00 (m, 2H).

Step D: 5-bromo-8-chlorothiochroman-4-ol To a solution of 5-bromo-8-chloro-3,4-dihydro-2H-1-benzothiin-4-one (600 mg, 2.162 mmol) in THF (10 mL) was added NaBH₄ (146 mg, 4.32 mmol). The reaction mixture was stirred at RT for 3 h. The reaction mixture was diluted with H₂O, extracted with EA, washed with brine, dried by Na₂SO₄ and concentrated. The residue was purified using silica gel column chromatography eluting with EA in PE [Gradient: 0-50%] afford the title compound 5-bromo-8-chloro-3,4-dihydro-2H-1-benzothiin-4-ol (400 mg, 1.43 mmol, 66.2%) as a colorless oil. No MS.

Step E: 5-bromo-8-chlorothiochromane To a solution of 5-bromo-8-chloro-3,4-dihydro-2H-1-benzothiin-4-ol (400 mg, 1.43 mmol) in TFA (5 mL) was added Et₃SiH (1.15 mL, 7.15 mmol). The reaction mixture was stirred at RT for 3 h. The reaction mixture was diluted with H₂O, extracted with EA, washed with brine, dried by Na₂SO₄ and concentrated. The residue was purified using silica gel column chromatography eluting with EA in PE [Gradient: 0-30%] afford the title compound 5-bromo-8-chloro-3,4-dihydro-2H-1-benzothiine (380 mg, 1.44 mmol, 89.5%) as a colorless oil. No MS, ¹H NMR (400 MHz, CDCl₃) δ 7.23 (d, J=8.6 Hz, 1H), 7.04 (d, J=8.6 Hz, 1H), 3.08-2.97 (m, 2H), 2.87 (dd, J=12.0, 6.0 Hz, 2H), 2.14 (dt, J=12.0, 6.0 Hz, 2H).

Step F: 5-bromo-8-chlorothiochromane 1,1-dioxide To a solution of 5-bromo-8-chloro-3,4-dihydro-2H-1-benzothiine (380 mg, 1.44 mmol) in DCE (5 mL) was added 3-chlorobenzene-1-carboperoxoic acid (497 mg, 2.88 mmol). The reaction mixture was stirred at 60° C. overnight. The cooled reaction mixture was quenched with Na₂S₂O₃, extracted with EA, dried by Na₂SO₄ and concentrated. The residue was purified using silica gel column chromatography eluting with EA in PE [Gradient: 0-40%] afford the title compound 5-bromo-8-chlorothiochromane 1,1-dioxide (310 mg, 1 mmol, 72%) as a white solid. No Ms

Step G: 5-((1-(tert-butyl)-3-((1S,3R)-3-((tert-butyldimethylsilyl)oxy)cyclopentyl)-1H-pyrazol-5-yl)amino)-8-chlorothiochromane 1,1-dioxide To a solution of 5-[(1S,3R)-3-{[dimethyl(2-methylprop-2-yl)silyl]oxy}cyclopentyl]-2-(2-methylprop-2-yl)pyrazol-3-amine (200 mg, 0.59 mmol) in dioxane (5 mL) were added 5-bromo-8-chlorothiochromane 1,1-dioxide (174 mg, 0.59 mmol), Cs₂CO₃ (386 mg, 1.18 mmol), Xantphos (68.5 mg, 0.12 mmol) and Pd₂(dba)₃ (54.2 mg, 0.06 mmol). The reaction mixture was stirred at 100° C. under N₂ overnight. The reaction mixture was diluted with H₂O, extracted with EA, washed with brine, dried by Na₂SO₄ and concentrated. The residue was purified using silica gel column chromatography eluting with EA in PE [Gradient: 0-50%] afford the title compound 5-((1-(tert-butyl)-3-((1S,3R)-3-((tert-butyldimethylsilyl)oxy)cyclopentyl)-1H-pyrazol-5-yl)amino)-8-chlorothiochromane 1,1-dioxide (120 mg, 0.22 mmol, 36%) as a yellow solid. LCMS: ESI m/z 552 [M+H]⁺

Step H: 5-((1-(tert-butyl)-3-((1S,3R)-3-hydroxycyclopentyl)-1H-pyrazol-5-yl)amino)-8-chlorothiochromane 1,1-dioxide A solution of 5-((1-(tert-butyl)-3-((1S,3R)-3-((tert-butyldimethylsilyl)oxy)cyclopentyl)-1H-pyrazol-5-yl)amino)-8-chlorothiochromane 1,1-dioxide (60 mg, 0.109 mmol) in HCOOH (3 mL) was stirred at room temperature for 1 hr. The reaction mixture was concentrated and were added MeOH (3 mL) and LiOH (10.5 mg, 0.582 mmol). The reaction mixture was stirred at room temperature for 30 min. The reaction was diluted with H₂O. The aqueous layer was extracted with EA, washed with saturated NaCl solution, dried by Na₂SO₄, filtered and concentrated. The residue was purified using silica gel column chromatography eluting with MeOH in DCM [gradient: 0-10%] to afford the title compound 5-((1-(tert-butyl)-3-((1S,3R)-3-hydroxycyclopentyl)-1H-pyrazol-5-yl)amino)-8-chlorothiochromane 1,1-dioxide (40 mg, 0.09 mmol, 84%) as a white solid. LCMS: ESI m/z 438 [M+H]⁺

Step I: (1R,3S)-3-(1-(tert-butyl)-5-((8-chloro-1,1-dioxidothiochroman-5-yl)amino)-1H-pyrazol-3-yl)cyclopentyl (4-nitrophenyl) carbonate To a solution of 5-((1-(tert-butyl)-3-((1S,3R)-3-hydroxycyclopentyl)-1H-pyrazol-5-yl)amino)-8-chlorothiochromane 1,1-dioxide (40 mg, 0.091 mmol) in DCM (3 mL) were added 4-nitrophenyl chloromethanoate (27.6 mg, 0.137 mmol), DMAP (1.1 mg, 0.009 mmol), pyridine (0.022 mL, 0.274 mmol). The reaction was stirred at RT for 3 hr. The reaction mixture was and concentrated. The residue was purified using silica gel column chromatography eluting with EA in PE [Gradient: 0-70%] to afford the title compound (1R,3S)-3-(1-(tert-butyl)-5-((8-chloro-1,1-dioxidothiochroman-5-yl)amino)-1H-pyrazol-3-yl)cyclopentyl (4-nitrophenyl) carbonate (40 mg, 0.066 mmol, 72%) as a white solid. LCMS: ESI m/z 603 [M+H]⁺

Step J: (1R,3S)-3-(3-((8-chloro-1,1-dioxidothiochroman-5-yl)amino)-1H-pyrazol-5-yl)cyclopentyl (4-nitrophenyl) carbonate A solution of (1R,3S)-3-(1-(tert-butyl)-5-((8-chloro-1,1-dioxidothiochroman-5-yl)amino)-1H-pyrazol-3-yl)cyclopentyl (4-nitrophenyl) carbonate (40 mg, 0.066 mmol) in HCOOH (2 mL) and H₂O (0.5 mL) was stirred at 100° C. overnight. The reaction mixture was concentrated to afford the title compound (1R,3S)-3-(3-((8-chloro-1,1-dioxidothiochroman-5-yl)amino)-1H-pyrazol-5-yl)cyclopentyl (4-nitrophenyl) carbonate (40 mg crude) as a yellow solid. LCMS: ESI m/z 547 [M+H]⁺

Step K: (1R,3S)-3-(3-((8-chloro-1,1-dioxidothiochroman-5-yl)amino)-1H-pyrazol-5-yl)cyclopentyl isopropylcarbamate A solution of (1R,3S)-3-(3-((8-chloro-1,1-dioxidothiochroman-5-yl)amino)-1H-pyrazol-5-yl)cyclopentyl (4-nitrophenyl) carbonate (40 mg, 0.073 mmol) in propan-2-amine (2 mL) was stirred at RT for 30 min. The reaction mixture was concentrated. The residue was purified by prep-HPLC to afford the title compound (1R,3S)-3-(3-((8-chloro-1,1-dioxidothiochroman-5-yl)amino)-1H-pyrazol-5-yl)cyclopentyl isopropylcarbamate (7 mg, 0.015 mmol, 20%) as a white solid. LCMS: ESI m/z 467[M+H]⁺¹HNMR(400 MHz, DMSO) δ 7.85 (d, J=8.8 Hz, 1H), 7.61 (s, 1H), 7.31 (d, J=8.8 Hz, 1H), 6.93 (d, J=7.2 Hz, 1H), 5.79 (s, 1H), 4.99 (s, 1H), 3.61-3.47 (m, 3H), 3.10-3.00 (m, 1H), 2.79 (s, 2H), 2.51-2.42 (m, 1H), 2.29 (brs, 2H), 2.07-1.96 (m, 1H), 1.95-1.87 (m, 1H), 1.76-1.66 (m, 2H), 1.59 (s, 1H), 1.65-1.53 (m, 1H), 1.03 (d, J=4.0 Hz, 6H).

### Example 59: (1R,3S)-3-(3-((1,1-dioxidothiochroman-5-yl)amino)-1H-pyrazol-5-yl)cyclopentyl isopropylcarbamate

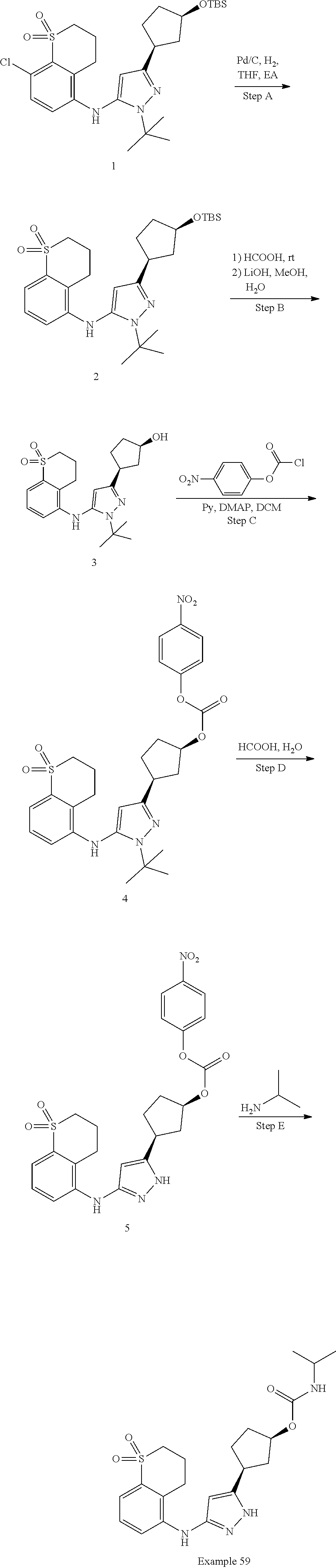

Step A: 5-((1-(tert-butyl)-3-((1S,3R)-3-((tert-butyldimethylsilyl)oxy)cyclopentyl)-1H-pyrazol-5-yl)amino)thiochromane 1,1-dioxide To a solution of 8-chloro-5-({5-[(1S,3R)-3-{[dimethyl(2-methylprop-2-yl)silyl]oxy}cyclopentyl]-2-(2-methylprop-2-yl)pyrazol-3-yl}amino)-3,4-dihydro-2H-1λ⁶-1-benzothiine-1,1-dione (60 mg, 0.109 mmol) in EA (1 mL) and THF (1 mL) were added Pd/C 10% (23.3 mg, 0.217 mmol). The reaction mixture was stirred at RT under H₂ balloon. The reaction mixture was filtered and concentrated to afford the title compound 5-((1-(tert-butyl)-3-((1S,3R)-3-((tert-butyldimethylsilyl)oxy)cyclopentyl)-1H-pyrazol-5-yl)amino)thiochromane 1,1-dioxide (60 mg crude) as a yellow solid. LCMS: ESI m/z 518[M+H]⁺

Step B: 5-((1-(tert-butyl)-3-((1S,3R)-3-hydroxycyclopentyl)-1H-pyrazol-5-yl)amino)thiochromane 1,1-dioxide A solution of 5-((1-(tert-butyl)-3-((1S,3R)-3-((tert-butyldimethylsilyl)oxy)cyclopentyl)-1H-pyrazol-5-yl)amino)thiochromane 1,1-dioxide (60 mg, 0.116 mmol) in HCOOH (3 mL) was stirred at room temperature for 1 hr. The reaction mixture was concentrated and were added MeOH (3 mL) and LiOH (42.4 mg, 0.232 mmol). The reaction mixture was stirred at room temperature for 30 min. The reaction was diluted with H₂O. The aqueous layer was extracted with EA, washed with saturated NaCl solution, dried by Na₂SO₄, filtered and concentrated. The residue was purified using silica gel column chromatography eluting with MeOH in DCM [gradient: 0-10%] to afford the title compound 5-((1-(tert-butyl)-3-((1S,3R)-3-hydroxycyclopentyl)-1H-pyrazol-5-yl)amino)thiochromane 1,1-dioxide (50 mg, 0.109 mmol, 94.1%) as a white solid. LCMS: ESI m/z 404 [M+H]⁺

Step C: (1R,3S)-3-(1-(tert-butyl)-5-((1,1-dioxidothiochroman-5-yl)amino)-1H-pyrazol-3-yl)cyclopentyl (4-nitrophenyl) carbonate To a solution of 5-((1-(tert-butyl)-3-((1S,3R)-3-hydroxycyclopentyl)-1H-pyrazol-5-yl)amino)thiochromane 1,1-dioxide (50 mg, 0.124 mmol) in DCM (3 mL) were added 4-nitrophenyl chloromethanoate (37.4 mg, 0.186 mmol), DMAP (4.5 mg, 0.037 mmol), pyridine (0.3 mL, 0.36 mmol). The reaction was stirred at RT for 3 hr. The reaction mixture was and concentrated. The residue was purified using silica gel column chromatography eluting with EA in PE [Gradient: 0-70%] to afford the title compound (1R,3S)-3-(1-(tert-butyl)-5-((1,1-dioxidothiochroman-5-yl)amino)-1H-pyrazol-3-yl)cyclopentyl (4-nitrophenyl) carbonate (42 mg, 0.074 mmol, 59.6%) as a white solid. LCMS: ESI m/z 568 [M+H]⁺

Step D: (1R,3S)-3-(3-((1,1-dioxidothiochroman-5-yl)amino)-1H-pyrazol-5-yl)cyclopentyl (4-nitrophenyl) carbonate A solution of (1R,3S)-3-(1-(tert-butyl)-5-((1,1-dioxidothiochroman-5-yl)amino)-1H-pyrazol-3-yl)cyclopentyl (4-nitrophenyl) carbonate (42 mg, 0.074 mmol) in HCOOH (2 mL) and H₂O (0.5 mL) was stirred at 100° C. overnight. The cooled reaction mixture was concentrated to afford the title compound (1R,3S)-3-(3-((1,1-dioxidothiochroman-5-yl)amino)-1H-pyrazol-5-yl)cyclopentyl (4-nitrophenyl) carbonate (42 mg crude) as a yellow solid. LCMS: ESI m/z 513 [M+H]⁺

Step E: (1R,3S)-3-(3-((1,1-dioxidothiochroman-5-yl)amino)-1H-pyrazol-5-yl)cyclopentyl isopropylcarbamate A solution of (1R,3S)-3-(3-((1,1-dioxidothiochroman-5-yl)amino)-1H-pyrazol-5-yl)cyclopentyl (4-nitrophenyl) carbonate (42 mg, 0.082 mmol) in propan-2-amine (2 mL) was stirred at RT for 30 min. The reaction mixture was concentrated. The residue was purified by prep-HPLC to afford the title compound (1R,3S)-3-(3-((1,1-dioxidothiochroman-5-yl)amino)-1H-pyrazol-5-yl)cyclopentyl isopropylcarbamate (7.1 mg, 0.016 mmol, 20%) as a white solid. LCMS: ESI m/z 433 [M+H]⁺. ¹H NMR (400 MHz, DMSO) δ 7.80 (brs, 1H), 7.63 (brs, 1H), 7.35-7.25 (m, 1H), 7.24-7.17 (m, 1H), 6.94 (brs, 1H), 5.80 (s, 1H), 4.99 (s, 1H), 3.63-3.54 (m, 1H), 3.45 (brs, 2H), 3.10-3.01 (m, 1H), 2.80 (s, 2H), 2.52-2.43 (m, 1H), 2.36 (brs, 2H), 2.16-1.98 (m, 1H), 1.96-1.87 (m, 1H), 1.76-1.65 (m, 2H), 1.63-1.53 (m, 1H), 1.03 (brs, 6H).

### Example 60: Synthesis of (1R,3S)-3-(3-((4-fluoro-1,1-dioxido-2,3-dihydrobenzo[b]thiophen-5-yl)amino)-1H-pyrazol-5-yl)cyclopentyl isopropylcarbamate

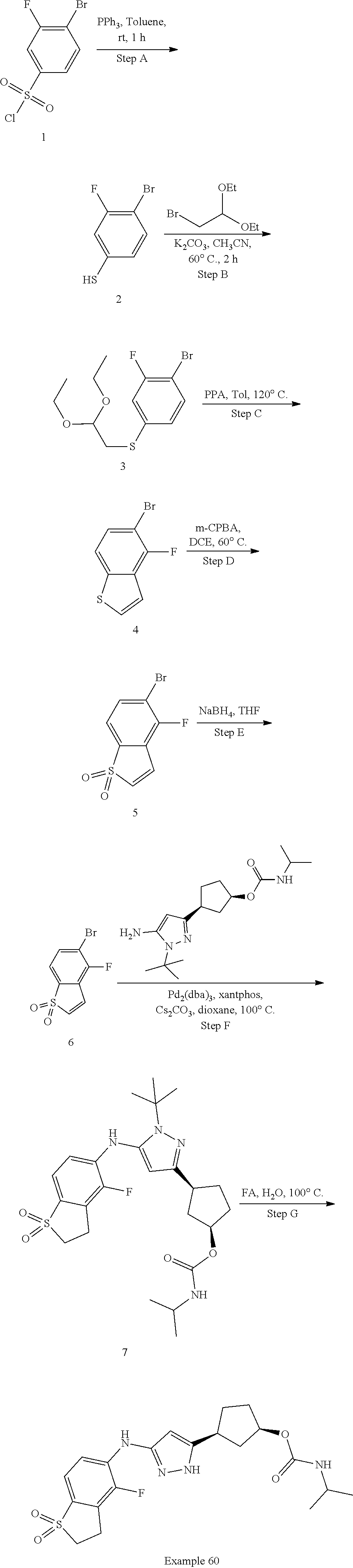

Step A. 4-bromo-3-fluorobenzene-1-thiol. To a stirred solution of 4-bromo-3-fluorobenzenesulfonyl chloride (5 g, 18.3 mmol) in toluene (50 mL) was added PPh₃ (14 g, 54.8 mmol) slowly at 0° C. After stirred at rt for 1 h, the cooled mixture was poured into water (30 mL) and extracted with toluene (20 mL). The combined organic phase was washed with 10% NaOH (aq, 50 mL*2). The combined NaOH aqueous phase was washed with toluene (30 mL), acidified with dilute HCl to PH 4-5, and extracted with DCM (40 mL*2). The combined organic phase was dried over anhydrous Na₂SO₄ and concentrated. The residue was purified by chromatography (silica gel, 0-5%, EtOAc in PE) to give 4-bromo-3-fluorobenzene-1-thiol (2.5 g, 12 mmol, 66%) as a yellow oil. No Ms.

Step B. 2-[(4-bromo-3-fluorophenyl)sulfanyl]-1,1-diethoxyethane. To a stirred solution of 4-bromo-3-fluorobenzene-1-thiol (2.5 g, 12 mmol) in CH₃CN (30 mL) was added 2-bromo-1,1-diethoxyethane (3.7 mL, 24.1 mmol) and K₂CO₃ (4.2 g, 30.2 mmol) slowly at rt. After stirring at 60° C. for 3 h, the cooled mixture was filtered and concentrated. The residue was purified by chromatography (silica gel, 0-5%, EtOAc in PE) to give 2-[(4-bromo-3-fluorophenyl)sulfanyl]-1,1-diethoxyethane (3.3 g, 10.2 mmol, 84%) as a colorless oil. No Ms

Step C. 5-bromo-4-fluorobenzo[b]thiophene. To a stirred solution of 2-[(4-bromo-3-fluorophenyl)sulfanyl]-1,1-diethoxyethane (2.5 g, 7.74 mmol) in toluene (80 mL) was added PPA (5 mL) at rt. After stirred at 120° C. for 5 h, the cooled mixture was filtered and concentrated. The residue was purified by chromatography (silica gel, 0-30%, EtOAc in PE) and then prep-HPLC (C18, 40˜ 90% MeCN in H₂O with 0.1% TFA) to give 5-bromo-4-fluorobenzothiophene (380 mg, 1.64 mmol, 21%) as a brown solid. No Ms.

Step D. 5-bromo-4-fluoro-1)<sup>6</sup>-benzo[b]thiophene-1,1-dione. To a stirred solution of 5-bromo-4-fluorobenzothiophene (380 mg, 1.6 mmol) in DCE (8 mL) was added m-CPBA (851 mg, 4.9 mmol) at rt. After stirring at 60° C. for 2 h, the cooled mixture was poured into NaHCO₃(20 mL) and extracted with DCM (10 mL*2). The combined organic phase was washed with brine, dried with Na₂SO₄, filtered and concentrated. The residue was purified by chromatography (silica gel, 0-40%, EtOAc in PE) to give 5-bromo-4-fluoro-1λ6-benzothiophene-1,1-dione (230 mg, 0.8 mmol, 53%) as a white solid. No Ms

Step E. 5-bromo-4-fluoro-2,3-dihydro-1λ<sup>6</sup>-benzo[b]thiophene-1,1-dione. To a stirred solution of 5-bromo-4-fluoro-1)⁶-benzothiophene-1,1-dione (230 mg, 0.8 mmol) in MeOH (2 mL)/DCM (2 mL) was added NaBH₄ (88 mg, 2.6 mmol) at 0° C. After stirring at rt for 2 h, the cooled mixture was poured into water (10 mL) and extracted with DCM (10 mL*2). The combined organic phase was washed with brine, dried with Na₂SO₄, filtered and concentrated. The residue was purified by chromatography (silica gel, 0-40%, EtOAc in PE) to give 5-bromo-4-fluoro-2,3-dihydro-1λ⁶-benzothiophene-1,1-dione (55 mg, 0.21 mmol, 24%) as a white solid. No Ms.

Step F. (1R,3S)-3-(1-(tert-butyl)-5-((4-fluoro-1,1-dioxido-2,3-dihydrobenzo[b]thiophen-5-yl)amino)-1H-pyrazol-3-yl)cyclopentyl isopropylcarbamate. To a stirred solution of 5-bromo-4-fluoro-2,3-dihydro-1⁶-benzothiophene-1,1-dione (55 mg, 0.21 mmol) in dioxane (5 mL) was added (1R,3S)-3-[5-amino-1-(2-methylprop-2-yl)pyrazol-3-yl]cyclopentyl (prop-2-ylamino)methanoate (40 mg, 0.13 mmol), Xant-PHOS (15 mg, 0.03 mmol), Pd₂(dba)₃ (12 mg, 0.01 mmol) and Cs₂CO₃ (105 mg, 0.3 mmol) at rt. After stirred at 100° C. for 2 h, the cooled mixture was poured into water (10 mL) and extracted with EtOAc (10 mL*2). The combined organic phase was washed with brine, dried with Na₂SO₄, filtered and concentrated. The residue was purified by chromatography (silica gel, 0-80%, EtOAc in PE) to give (1R,3S)-3-(1-(tert-butyl)-5-((4-fluoro-1,1-dioxido-2,3-dihydrobenzo[b]thiophen-5-yl)amino)-1H-pyrazol-3-yl)cyclopentyl isopropylcarbamate (45 mg, 0.09 mmol, 42%) as a yellow solid. LCMS: m/z 493 [M+H]⁺.

Step G. (1R,3S)-3-(3-((4-fluoro-1,1-dioxido-2,3-dihydrobenzo[b]thiophen-5-yl)amino)-1H-pyrazol-5-yl)cyclopentyl isopropylcarbamate. To a stirred solution of (1R,3S)-3-(1-(tert-butyl)-5-((4-fluoro-1,1-dioxido-2,3-dihydrobenzo[b]thiophen-5-yl)amino)-1H-pyrazol-3-yl)cyclopentyl isopropylcarbamate (45 mg, 0.09 mmol) in HCOOH (3 mL) was added H₂O (0.5 mL, 55.5 mmol) at rt. After stirring at 100° C. overnight, the cooled mixture was concentrated. The residue was purified by prep-HPLC (C18, 40˜ 90% MeCN in H₂O with 0.1% TFA) to give (1R,3S)-3-(3-((4-fluoro-1,1-dioxido-2,3-dihydrobenzo[b]thiophen-5-yl)amino)-1H-pyrazol-5-yl)cyclopentyl isopropylcarbamate (9 mg, 0.02 mmol, 22%) as a white solid. LCMS: m/z 437 [M+H]⁺. ¹H NMR (400 MHz, DMSO) δ 11.98 (s, 1H), 8.70 (s, 1H), 8.26-8.21 (m, 1H), 7.41 (d, J=8.4 Hz, 1H), 6.94 (d, J=7.2 Hz, 1H), 5.83 (s, 1H), 4.99 (brs, 1H), 3.56 (t, J=6.0 Hz, 3H), 3.09-3.01 (m, 1H), 2.48-2.41 (m, 1H), 2.06-1.99 (m, 1H), 1.99-1.88 (m, 1H), 1.72-1.54 (m, 3H), 1.03 (d, J=5.6 Hz, 6H).

### Example 61: Synthesis of (1R,3S)-3-(3-((1,1-dioxido-3,4-dihydro-2H-benzo[e][1,2]thiazin-5-yl)amino)-1H-pyrazol-5-yl)cyclopentyl isopropylcarbamate

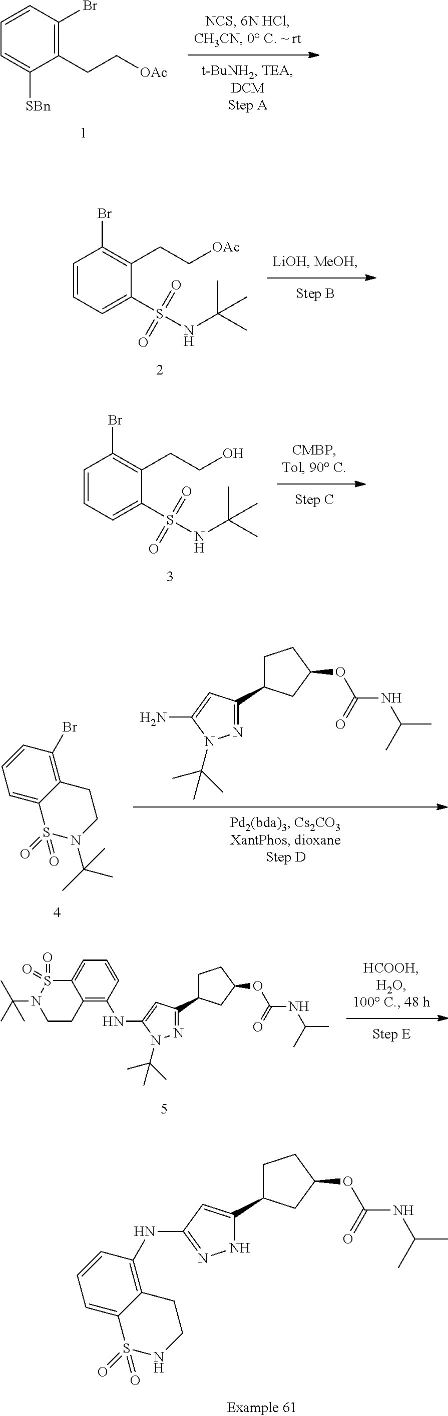

Step A: 2-(2-bromo-6-{[(2-methylprop-2-yl)amino]dioxo-λ6-sulfanyl}phenyl)ethyl acetate To a stirred suspension of 2-[6-(benzylsulfanyl)-2-bromophenyl]ethyl acetate (600 mg, 1.6 mmol) in CH₃CN (8 mL) and 6N HCl (8 ml) was added NCS (1096 mg, 8.2 mmol) at 0° C. The reaction mixture was allowed to warm to ambient temperature and stirred for 2 h. The reaction was partitioned between EtOAc and water. The aqueous layer was extracted with EtOAc. The combined organic layers were washed with water and brine, dried and concentrated to give a crude 2-[2-bromo-6-(chlorodioxo-λ⁶-sulfanyl)phenyl]ethyl acetate. The above crude product dissolved in DCM (5 mL) and added into a solution of tert-butylamine (180 mg, 2.46 mmol) and DIEA (244 mg, 1.9 mmol) in DCM (5 mL) at 0° C. The reaction mixture was allowed to warm to ambient temperature and stirred for 2 hours. The reaction was partitioned between DCM and water. The aqueous layer was extracted with DCM. The combined organic layer was washed with water and brine, dried and concentrated. The residue was purified by silica gel column chromatography eluted with ethyl acetate in petroleum ether. (Gradient: 0-40%) to afford the title compound 2-(2-bromo-6-{[(2-methylprop-2-yl)amino]dioxo-1λ6-sulfanyl}phenyl)ethyl acetate (400 mg, 1.0 mmol, 64%) as a colorless oil. LCMS: m/z 378 [M+H]⁺.

Step B: 3-bromo-2-(2-hydroxyethyl)-N-(2-methylprop-2-yl)benzenesulfonamide A solution of 2-(2-bromo-6-{[(2-methylprop-2-yl)amino]dioxo-λ⁶-sulfanyl}phenyl)ethyl acetate (400 mg, 1.0 mmol) and LiOH (210 mg, 5.0 mmol) in MeOH (5 mL) and H₂O (5 mL) was a stirred at room temperature for 2 h. The reaction was partitioned between EtOAc and water. The aqueous layer was extracted with EtOAc. The combined organic layers were washed with water and brine, dried and concentrated. The residue was purified by silica gel column chromatography eluting with ethyl acetate in petroleum ether. (gradient: 0˜ 40%) to afford the title compound 3-bromo-2-(2-hydroxyethyl)-N-(2-methylprop-2-yl)benzenesulfonamide (240 mg, 0.71 mmol, 67.5%) as a colorless oil. LCMS: m/z 336 [M+H]⁺

Step C: 5-bromo-2-(2-methylprop-2-yl)-3,4-dihydro-2H-1λ6-benzo[2,1-e][1,2]thiazine-1,1-dione A solution of 3-bromo-2-(2-hydroxyethyl)-N-(2-methylprop-2-yl)benzenesulfonamide (240 mg, 0.7 mmol) and 2-(tributylphosphoranylidene)acetonitrile (258 mg, 1.0 mmol) in toluene (5 mL) was stirred at 100° C. under N₂ atmosphere for 2 h. TLC and LCMS indicated the reaction was over. The mixture was concentrated and the residue was purified by FC (gradient: 0˜ 30% EA in PE) to give the desire product 5-bromo-2-(2-methylprop-2-yl)-3,4-dihydro-2H-1λ⁶-benzo[2,1-e][1,2]thiazine-1,1-dione (100 mg, 0.31 mmol, 44%) as an off-white solid.

Step D: (1R,3S)-3-(1-(tert-butyl)-5-((2-(tert-butyl)-1,1-dioxido-3,4-dihydro-2H-benzo[e][1,2]thiazin-5-yl)amino)-1H-pyrazol-3-yl)cyclopentyl isopropylcarbamate To a solution of 5-bromo-2-(2-methylprop-2-yl)-3,4-dihydro-2H-1λ⁶-benzo[2,1-e][1,2]thiazine-1,1-dione (100 mg, 0.31 mmol), (1R,3S)-3-[5-amino-1-(2-methylprop-2-yl)pyrazol-3-yl]cyclopentyl (prop-2-ylamino)methanoate (97 mg, 0.31 mmol), Xantphos (18 mg, 0.031 mmol) and Cs₂CO₃ (204 mg, 0.63 mmol) in dioxane (8 mL) was added Pd₂dba₃ (29 mg, 0.031 mmol). The reaction was stirred at 100° C. under N₂ atmosphere for 5 h. TLC and LCMS indicted the reaction was over. The cooled reaction mixture was poured into ice-water, extracted with EA (20 mL*3), the combined organic layer was washed with brine, dried over Na₂SO₄, filtered and concentrated. The residue was purified by a silica column eluted with EA in PE (gradient: 0-50%) to give the desire product (1R,3S)-3-(1-(tert-butyl)-5-((2-(tert-butyl)-1,1-dioxido-3,4-dihydro-2H-benzo[e][1,2]thiazin-5-yl)amino)-1H-pyrazol-3-yl)cyclopentyl isopropylcarbamate (80 mg, 0.15 mmol, 46%) as a yellow solid. LCMS m/z: 546 [M+H]⁺

Step E: (1R,3S)-3-(3-((1,1-dioxido-3,4-dihydro-2H-benzo[e][1,2]thiazin-5-yl)amino)-1H-pyrazol-5-yl)cyclopentyl isopropylcarbamate A solution of (1R,3S)-3-(1-(tert-butyl)-5-((2-(tert-butyl)-1,1-dioxido-3,4-dihydro-2H-benzo[e][1,2]thiazin-5-yl)amino)-1H-pyrazol-3-yl)cyclopentyl isopropylcarbamate (60 mg, 0.11 mmol) in HCOOH (4 mL) and H₂O (0.4 mL) was stirred at 100° C. for 18 h. TLC and LCMS indicated the reaction was over. The reaction mixture was concentrated and the residue was purified by prep-HPLC to give the desire product (1R,3S)-3-{5-[(1,1-dioxo-3,4-dihydro-2H-1λ⁶-benzo[2,1-e][1,2]thiazin-5-yl)amino]-1H-pyrazol-3-yl}cyclopentyl (prop-2-ylamino)methanoate (7 mg, 0.016 mmol, 14.7%) as a white solid. LCMS m/z: 434 [M+H]⁺. ¹H NMR (400 MHz, DMSO) δ 7.81-7.75 (m, 1H), 7.57 (s, 1H), 7.37-7.22 (m, 2H), 7.13 (d, J=8.0 Hz, 1H), 6.93 (d, J=7.6 Hz, 1H), 5.78 (s, 1H), 4.99 (brs, 1H), 3.62-3.50 (m, 3H), 3.12-2.97 (m, 1H), 2.75-2.64 (m, 2H), 2.47-2.42 (m, 1H), 2.05-1.97 (m, 1H), 1.95-1.87 (m, 1H), 1.77-1.57 (m, 3H), 1.02 (d, J=5.2 Hz, 6H).

### Example 62: (1R,3S)-3-(5-((4-hydroxy-2,2-dioxidoisothiochroman-8-yl)amino)-1H-pyrazol-3-yl)cyclopentyl isopropylcarbamate

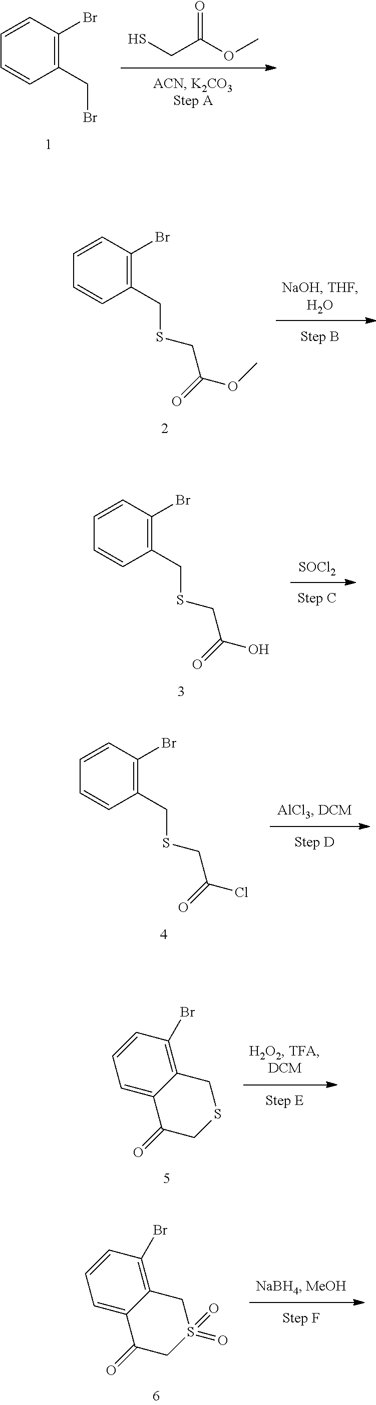

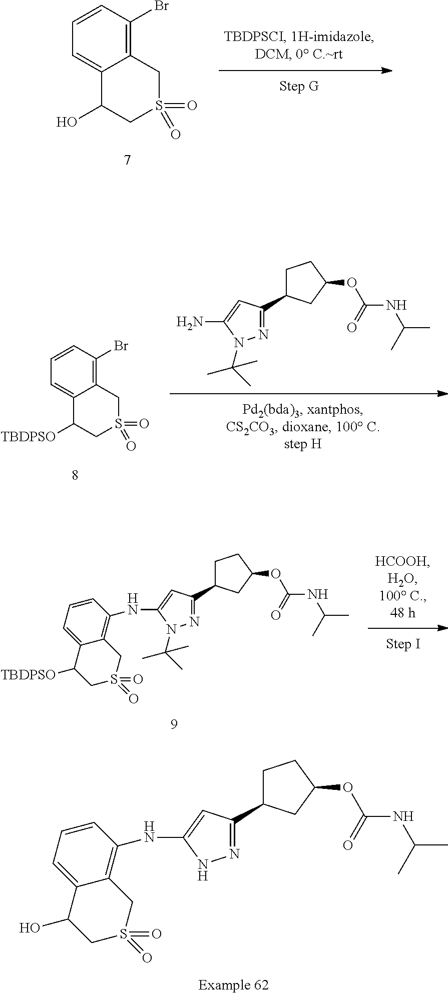

Step A: methyl {[(2-bromophenyl)methyl]sulfanyl}acetate To a solution of 1-bromo-2-(bromomethyl)benzene (5 g, 20.0 mmol) in acetonitrile (50 mL) was added K₂CO₃ (5.53 g, 40.0 mmol) and methyl sulfanylacetate (1.8 mL, 20.0 mmol) at rt. The reaction mixture was stirred at rt overnight. The reaction was diluted with EA and water. The organic layer was separated, washed with brine, dried over Na₂SO₄ and concentrated in vacuo. The residue was purified using silica gel column chromatography eluted with ethyl acetate in petroleum ether. (gradient:0-8%) to afford the title compound methyl {[(2-bromophenyl)methyl]sulfanyl}acetate (4.5 g, 16.4 mmol, 81.7%) as a colorless oil. LCMS: ESI m/z 275 [M+H]⁺.

Step B: {[(2-bromophenyl)methyl]sulfanyl}acetic acid To a solution of methyl {[(2-bromophenyl)methyl]sulfanyl}acetate (4.5 g, 16.4 mmol) in THF (50 mL) was added dropwise a solution of NaOH (3.27 g, 81.7 mmol) in H₂O (10 mL). The reaction mixture was stirred at rt for 4 hr. The mixture was adjusted pH to 5 by concentrated hydrochloric acid, extracted with EA. The organic layer was separated, washed with brine, dried over Na₂SO₄ and concentrated in vacuo. The residue was purified by silica gel column chromatography eluted with ethyl acetate in petroleum ether (0˜35% gradient) to afford the title compound {[(2-bromophenyl)methyl]sulfanyl}acetic acid (4 g, 15.3 mmol, 93.66%) as a colorless oil. LCMS: ESI m/z 261 [M+H]⁺.

Step C: {[(2-bromophenyl)methyl]sulfanyl}acetyl chloride To a stirred mixture of {[(2-bromophenyl)methyl]sulfanyl}acetic acid (3 g, 11.5 mmol) in dichloromethane (30 mL) was added dropwise a solution of SOCl₂ (4.2 mL, 57.4 mmol) in dichloromethane (5 mL) over 10 min at 0° C. The reaction mixture was stirred at room temperature for 1 h. The reaction mixture was concentrated in vacuo to obtain {[(2-bromophenyl)methyl]sulfanyl}acetyl chloride (3 g, 10.7 mmol, 93.4%) as a colorless oil.

Step D: 8-bromo-3,4-dihydro-1H-2-benzothiin-4-one To a solution of {[(2-bromophenyl)methyl]sulfanyl}acetyl chloride (3 g, 10.7 mmol) in DCM (30 mL) was added aluminium chloride hexahydrate (3.11 g, 12.9 mmol) at 0° C. The reaction mixture was stirred at rt for 4 h. A solution of con. hydrochloric acid in water (50:50, 80 mL) was added to the reaction mixture. Partition the two layers and wash the organic layer with saturated sodium bicarbonate solution, water and brine in turn. The organic layer was dried over anhydrous Na₂SO₄, filtered and concentrated in vacuo. The residue was purified by column chromatography on silica gel with EA in PE (0-6%, V/V) to give 8-bromo-3,4-dihydro-1H-2-benzothiin-4-one (520 mg, 2.14 mmol, 20%) as a yellow solid. LCMS: ESI m/z 243 [M+H]⁺. ¹H NMR (400 MHz, CDCl3) δ 7.98 (d, J=7.6 Hz, 1H), 7.66 (d, J=8.0 Hz, 1H), 7.26-7.17 (m, 1H), 3.93 (s, 2H), 3.42 (s, 2H).

Step E: 8-bromoisothiochroman-4-one 2,2-dioxide To a stirred suspension of 8-bromo-3,4-dihydro-1H-2-benzothiin-4-one (2.1 g, 8.6 mmol) and TFA (4 mL) in DCM (20 mL) was added H₂O₂(4 mL, 39.2 mmol, 30% in H₂O) at 0° C. The reaction mixture was allowed to warm to ambient temperature and stirred for 2 hours. The reaction solution was poured into ice water and quenched with Na₂S₂O₃, extracted with DCM (20 mL*3). The combined organic layers were washed with brine, dried and concentrated. The residue was purified by a silica column eluted with EA in PE (0-6%, V/V) (gradient: 0-50%) to give the desire product 8-bromoisothiochroman-4-one 2,2-dioxide (1.4 g, 5.0 mmol, 59%) as a yellow solid. LCMS: ESI m/z 274 [M+H]⁺

Step F: 8-bromo-4-hydroxyisothiochromane 2,2-dioxide To a stirred suspension of 8-bromoisothiochroman-4-one 2,2-dioxide (2.1 g, 7.6 mmol) in MeOH (30 mL) was added NaBH₄ (390 mg, 11.4 mmol) at 0° C. The reaction mixture was allowed to warm to ambient temperature and stirred for 1 hour. The reaction was concentrated, and the residue was partitioned between EtOAc and water. The aqueous layer was extracted with EtOAc. The combined organic layers were washed with water and brine, dried and concentrated. The residue was purified by a silica column eluted with EA in PE (gradient: 0-50%) to give the desire product 8-bromo-4-hydroxyisothiochromane 2,2-dioxide (1.6 g, 5.8 mmol, 75%) as a colorless oil. LCMS: ESI m/z 274 [M+H]⁺. ¹H NMR (400 MHz, DMSO) δ 7.68 (d, J=8.0 Hz, 1H), 7.60 (d, J=7.6 Hz, 1H), 7.42-7.33 (m, 1H), 6.34 (d, J=5.2 Hz, 1H), 5.26-4.91 (m, 1H), 4.60 (d, J=15.2 Hz, 1H), 4.46 (d, J=15.2 Hz, 1H), 3.59 (dd, J=13.2, 5.2 Hz, 1H), 3.39-3.30 (m, 1H).

Step G: 8-bromo-4-((tert-butyldiphenylsilyl)oxy)isothiochromane 2,2-dioxide To a stirred suspension of 8-bromo-4-hydroxyisothiochromane 2,2-dioxide (600 mg, 2.1 mmol) and imidazole (221 mg, 3.2 mmol) in DCM (15 mL) was added tert-Butylchlorodiphenylsilane (771 mg, 2.8 mmol) at 0° C. The reaction mixture was allowed to warm to ambient temperature and stirred for 2 hours. The reaction was partitioned between DCM and water. The aqueous layer was extracted with DCM. The combined organic layers were washed with water and brine, dried and concentrated. The residue was purified a silica column eluted with EA in PE (gradient: 0-30%) give 8-bromo-4-((tert-butyldiphenylsilyl)oxy)isothiochromane 2,2-dioxide (510 mg, 0.98 mmol, 45.7%) as a colorless oil. ¹H NMR (400 MHz, DMSO) δ 7.75-7.65 (m, 3H), 7.59-7.43 (m, 6H), 7.42-7.31 (m, 2H), 7.27 (t, J=7.6 Hz, 1H), 7.16 (d, J=7.6 Hz, 1H), 5.31 (s, 1H), 4.64 (s, 2H), 3.50 (dd, J=13.6, 5.2 Hz, 1H), 3.27 (brd, J=13.6 Hz, 1H), 1.01 (s, 9H).

Step H: (1R,3S)-3-(1-(tert-butyl)-5-((4-((tert-butyldiphenylsilyl)oxy)-2,2-dioxidoisothiochroman-8-yl)amino)-1H-pyrazol-3-yl)cyclopentyl isopropylcarbamate To a solution of 8-bromo-4-((tert-butyldiphenylsilyl)oxy)isothiochromane (430 mg, 0.86 mmol), (1R,3S)-3-[5-amino-1-(2-methylprop-2-yl)pyrazol-3-yl]cyclopentyl (prop-2-ylamino)methanoate (265 mg, 0.86 mmol), Xantphos (501 mg, 0.086 mmol) and Cs₂CO₃ (841 mg, 2.58 mmol) in dioxane (8 mL) was added Pd₂(dba)₃ (79 mg, 0.086 mmol). The reaction was stirred at 100° C. under N₂ atmosphere for 5 h. TLC and LCMS indicted the reaction was over. The cooled reaction was poured into ice-water, extracted with EA (20 mL*3). The combined organic layer was washed with brine, dried over Na₂SO₄, filtered and concentrated. The residue was purified by a silica column eluted with EA in PE (gradient: 0˜ 50%) to give (1R,3S)-3-(1-(tert-butyl)-5-((4-((tert-butyldiphenylsilyl)oxy)-2,2-dioxidoisothiochroman-8-yl)amino)-1H-pyrazol-3-yl)cyclopentyl isopropylcarbamate (260 mg, 0.35 mmol, 40.6%) as a yellow solid.

Step I: (1R,3S)-3-(5-((4-hydroxy-2,2-dioxidoisothiochroman-8-yl)amino)-1H-pyrazol-3-yl)cyclopentyl isopropylcarbamate A solution of (1R,3S)-3-(1-(tert-butyl)-5-((4-((tert-butyldiphenylsilyl)oxy)-2,2-dioxidoisothiochroman-8-yl)amino)-1H-pyrazol-3-yl)cyclopentyl isopropylcarbamate (30 mg, 0.04 mmol) in HCOOH (3 mL) and H₂O (0.3 mL) was stirred at 100° C. for 48 h. The cooled reaction was concentrated and the residue was dissolved in MeOH (3 mL). 1 N LiOH (3 mL) was added and stirred for 30 min. MeOH was removed by reduce power and extracted with EA (5 mL*3), the combined organic layer was washed with brine, dried over Na₂SO₄, filtered and concentrated. The residue was purified by prep-HPLC to afford (1R,3S)-3-(5-((4-hydroxy-2,2-dioxidoisothiochroman-8-yl)amino)-1H-pyrazol-3-yl)cyclopentyl isopropylcarbamate (6.8 mg, 0.015 mmol, yield 37%) as an off-white solid. LCMS: 448 [M+H]⁺. ¹H NMR (400 MHz, DMSO) δ 7.91 (brs, 1H), 7.54 (d, J=7.6 Hz, 1H), 7.24 (t, J=7.6 Hz, 1H), 7.06 (d, J=8.8 Hz, 1H), 6.93 (d, J=8.8 Hz, 1H), 5.71 (s, 1H), 5.10 (s, 1H), 4.99 (s, 1H), 4.48 (d, J=15.6 Hz, 1H), 4.38 (d, J=15.6 Hz, 1H), 3.58-3.43 (m, 2H), 3.36-3.21 (m, 1H), 3.15-2.97 (m, 1H), 2.51-2.42 (m, 1H), 2.11-1.85 (m, 2H), 1.80-1.47 (m, 3H), 1.02 (d, J=2.8 Hz, 6H)

### Example 63: (1R,3S)-3-(3-((2,2-dioxidoisothiochroman-8-yl)amino)-1H-pyrazol-5-yl)cyclopentyl isopropylcarbamate

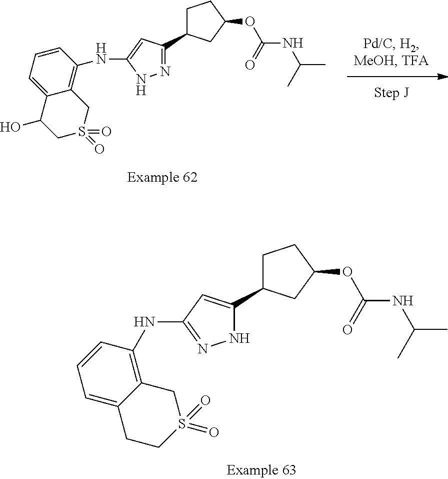 [SMILES: CC(C)NC(=O)O[C@@H]1CC[C@H](c2cc(Nc3cccc4c3CS(=O)(=O)CC4O)[nH]n2)C1 ; CC(C)NC(=O)O[C@@H]1CC[C@H](c2cc(Nc3cccc4c3CS(=O)(=O)CC4)n[nH]2)C1]

A suspension of (1R,3S)-3-(5-((4-hydroxy-2,2-dioxidoisothiochroman-8-yl)amino)-1H-pyrazol-3-yl)cyclopentyl isopropylcarbamate (30 mg, 0.067 mmol) and Pd/C 10% (10 mg) in MeOH (5 mL) and TFA (1 mL) was stirred at 50° C. under H₂ atmosphere overnight. The reaction was filtered and concentrated. The residue was purified by prep-HPLC to afford (1R,3S)-3-(3-((2,2-dioxidoisothiochroman-8-yl)amino)-1H-pyrazol-5-yl)cyclopentyl isopropylcarbamate (11 mg, 0.025 mmol, 38%) as a white solid. LCMS: 433 [M+H]⁺. ¹H NMR (400 MHz, MeOD) δ 7.38 (dd, J=7.6 Hz, 7.6 Hz, 1H), 7.29 (d, J=7.6 Hz, 1H), 7.22 (d, J=7.6 Hz, 1H), 5.08 (s, 1H), 4.31 (s, 2H), 3.74-3.61 (m, 1H), 3.45 (t, J=6.4 Hz, 2H), 3.36 (t, J=6.4 Hz, 2H), 3.23-3.14 (m, 1H), 2.61-2.48 (m, 1H), 2.19-2.08 (m, 1H), 1.98-1.75 (m, 4H), 1.10 (t, J=6.4 Hz, 6H).

### Example 64: (1R,3S)-3-(3-((7-chloro-1,1-dioxido-2,3-dihydrobenzo[b]thiophen-4-yl)amino)-1H-pyrazol-5-yl)cyclopentyl isopropylcarbamate

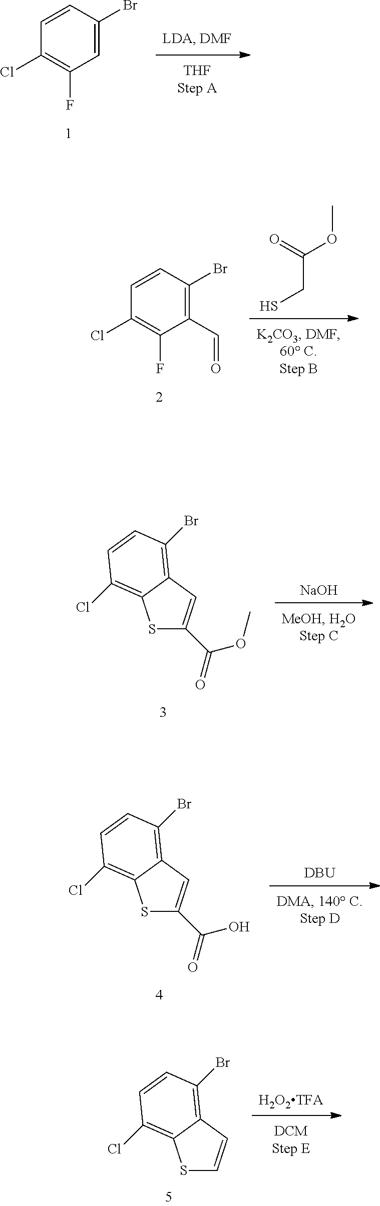

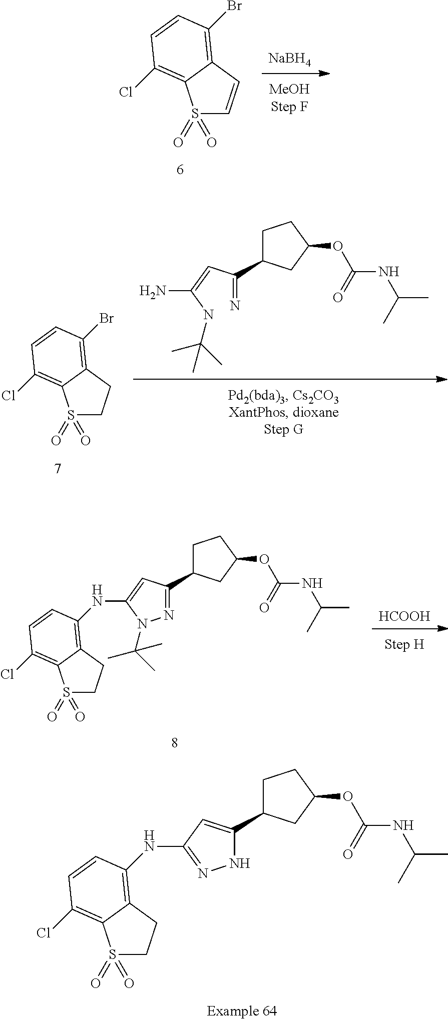

Step A: 6-bromo-3-chloro-2-fluorobenzene-1-carbaldehyde To a stirred mixture of 4-bromo-1-chloro-2-fluorobenzene (2 g, 9.55 mmol) in tetrahydrofuran (10 mL) was added dropwise LDA (1.5 mL, 28.6 mmol) at −60° C. under N₂ atmosphere. The mixture was stirred at −60° C. for additional 0.5 h under N₂ atmosphere. Then DMF (1.47 mL, 19.1 mmol) was added dropwise. The resulting mixture was stirred at RT for additional 1 h. The reaction was quenched with saturated NH₄Cl (aq) under an ice bath. The resulting mixture was extracted with EA. The combined organic layers were washed with brine, dried over anhydrous Na₂SO₄. After filtration, the filtrate was concentrated under reduced pressure. The residue was purified using silica gel column chromatography eluting with EA in PE [Gradient: 8%] to afford the title compound 6-bromo-3-chloro-2-fluorobenzene-1-carbaldehyde (1.8 g, 7.58 mmol, 79%) as a white solid. ¹H NMR (400 M Hz, DMSO) δ 10.16 (s, 1H), 7.82 (t, J=8.0 Hz, 1H), 7.68 (d, J=8.0 Hz, 1H)

Step B: methyl 4-bromo-7-chlorobenzothiophene-2-carboxylate To a solution of 6-bromo-3-chloro-2-fluorobenzene-1-carbaldehyde (1.8 g, 7.58 mmol) in DMF (15 mL) were added K₂CO₃ (2.1 g, 15.1 mmol), and methyl sulfanylacetate (1.01 mL, 11.4 mmol). The reaction mixture was stirred at 60° C. under N₂ overnight. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 7%] to afford the title compound methyl 4-bromo-7-chlorobenzothiophene-2-carboxylate (1.9 g, 6.21 mmol, 82%) as a white solid. ¹H NMR (400 MHz, DMSO) δ 8.06 (s, 1H), 7.80 (d, J=8.0 Hz, 1H), 7.64 (d, J=8.0 Hz, 1H), 3.94 (s, 3H).

Step C: 4-bromo-7-chlorobenzothiophene-2-carboxylic acid To a solution of methyl 4-bromo-7-chlorobenzothiophene-2-carboxylate (1.8 g, 5.891 mmol) in MeOH (5 mL). Then a solution of NaOH (0.94 g, 23.6 mmol) in H₂O (5 mL) was added dropwise. The reaction mixture was stirred at RT for 4 hr. The mixture was adjusted pH to 5 by concentrated hydrochloric acid, extracted with EA. The organic layer was separated, washed with brine, dried over Na₂SO₄, filtered, and concentrated in vacuo. The residue was purified using silica gel column chromatography eluting with MeOH in DCM [Gradient: 5%] to afford the title compound {4-bromo-7-chlorobenzothiophene-2-carboxylic acid (1.4 g, 4.80 mmol, 81%) as a white solid. LCMS: ESI m/z 291 [M+H]⁺

Step D: 4-bromo-7-chlorobenzothiophene To a solution of 4-bromo-7-chlorobenzothiophene-2-carboxylic acid (1.4 g, 4.8 mmol) in DMA (8 mL) were added DBU (3.4 mL, 24.1 mmol). The reaction mixture was stirred at 140° C. for 1 hr with microwave. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 5%] to afford the title compound 4-bromo-7-chlorobenzothiophene (600 mg, 2.42 mmol, 50%) as a white solid.

Step E: 4-bromo-7-chloro-1)⁶-benzothiophene-1,1-dione To a stirred solution of 4-bromo-7-chlorobenzothiophene (600 mg, 2.42 mmol) in DCM (8 mL) were added H₂O₂(2 mL, 19.6 mmol, 30% in H₂O) and TFA (2 mL, 26.9 mmol) at room temperature. After stirred at room temperature for 5 h, the mixture was poured into Na₂S₂O₃ (aq) and extracted with DCM. The combined organic phase was washed with brine, dried over Na₂SO₄, filtered and concentrated. The residue was purified by chromatography eluting with EA in PE [Gradient: 10%] to give 4-bromo-7-chloro-1λ⁶-benzothiophene-1,1-dione (400 mg, 1.43 mmol, 59%) as a white solid. NO LCMS. ¹H NMR (400 MHz, CDCl₃) δ 7.62 (d, J=8.6 Hz, 1H), 7.36 (d, J=7.2 Hz, 1H), 7.32 (d, J=8.6 Hz, 1H), 6.83 (d, J=7.2 Hz, 1H).

Step F: 4-bromo-7-chloro-2,3-dihydro-1λ⁶-benzothiophene-1,1-dione To a solution of 4-bromo-7-chloro-1λ6-benzothiophene-1,1-dione (650 mg, 2.32 mmol) in MeOH (5 mL) was added NaBH₄ (115 mg, 13.9 mmol) at 0° C. The mixture was stirred at room temperature for 2 hrs. The reaction was complete detected by LCMS. The residue was concentrated and dissolved in EA, washed with H₂O and brine, dried over Na₂SO₄, filtered, concentrated, and purified by silica gel chromatography eluting with EA in PE [Gradient:8%] to afford compound 4-bromo-7-chloro-2,3-dihydro-1,6-benzothiophene-1,1-dione (270 mg, 0.95 mmol, 41%) as a white solid. No MS. ¹H NMR (400 MHz, CDCl₃) δ 7.69 (d, J=8.4 Hz, 1H), 7.31 (d, J=8.4 Hz, 1H), 3.60 (t, J=7.2 Hz, 2H), 3.30 (t, J=7.02 Hz, 2H).

Step G: (1R,3S)-3-{5-[(7-chloro-1,1-dioxo-2,3-dihydro-1λ⁶-benzothiophen-4-yl)amino]-1-(2-methylprop-2-yl)pyrazol-3-yl}cyclopentyl (prop-2-ylamino)methanoate To a solution of 4-bromo-7-chloro-2,3-dihydro-1λ⁶-benzothiophene-1,1-dione (270 mg, 0.959 mmol) in dioxane (8 mL) were added (1R,3S)-3-[5-amino-1-(2-methylprop-2-yl)pyrazol-3-yl]cyclopentyl (prop-2-ylamino)methanoate (246 mg, 0.799 mmol), Xant-PHOS (92 mg, 0.16 mmol), Cs₂CO₃ (520 mg, 1.5 mmol) and Pd₂(dba)₃ (73 mg, 0.08 mmol). The reaction mixture was stirred at 100° C. under N₂ overnight for 2 h. The cooled reaction mixture was diluted with H₂O, extracted with EA. The organic phase was washed with brine, dried by Na₂SO₄, filtered and concentrated. The residue was purified using silica gel column chromatography eluting with EA in PE [Gradient: 70%] to afford the title compound (1R,3S)-3-{5-[(7-chloro-1,1-dioxo-2,3-dihydro-1λ⁶-benzothiophen-4-yl)amino]-1-(2-methylprop-2-yl)pyrazol-3-yl}cyclopentyl (prop-2-ylamino)methanoate (400 mg, 0.786 mmol, 98.3%) as a white solid. LCMS: ESI m/z 509 [M+H]⁺.

Step H: (1R,3S)-3-(3-((7-chloro-1,1-dioxido-2,3-dihydrobenzo[b]thiophen-4-yl)amino)-1H-pyrazol-5-yl)cyclopentyl isopropylcarbamate A solution of 2-methyl-4-{[2-(2-methylprop-2-yl)-5-[(1S,3R)-3-{[4-(prop-2-yl)-1,2-diazin-3-yl]oxy}cyclopentyl]pyrazol-3-yl]amino}-2,3-dihydro-1λ6-benzo[2,1-d][1,2]thiazole-1,1-dione (50 mg, 0.095 mmol) in HCOOH (3 mL) and H₂O (1 mL) was stirred 100° C. overnight. The reaction mixture was concentrated. The residue was purified by prep-HPLC to afford the title compound (1R,3S)-3-(3-((7-chloro-1,1-dioxido-2,3-dihydrobenzo[b]thiophen-4-yl)amino)-1H-pyrazol-5-yl)cyclopentyl isopropylcarbamate (14.8 mg, 0.033 mmol, 33%) as a white solid. LCMS: ESI m/z 453 [M+H]⁺. ¹H NMR (400 MHz, DMSO) δ 11.97 (s, 1H), 8.14-7.98 (m, 2H), 7.36 (d, J=9.2 Hz, 1H), 6.95 (brs, 1H), 5.80 (s, 1H), 4.99 (brs, 1H), 3.77-3.64 (m, 2H), 3.63-3.51 (m, 1H), 3.26-3.14 (m, 2H), 3.10-3.00 (m, 1H), 2.10-2.01 (m, 1H), 1.95-1.84 (m, 1H), 1.78-1.66 (m, 2H), 1.65-1.56 (m, 1H), 1.03 (d, J=5.6 Hz, 6H).

### Example 65: (1R,3S)-3-(3-((7-methyl-1,1-dioxido-2,3-dihydrobenzo[b]thiophen-4-yl)amino)-1H-pyrazol-5-yl)cyclopentyl isopropylcarbamate

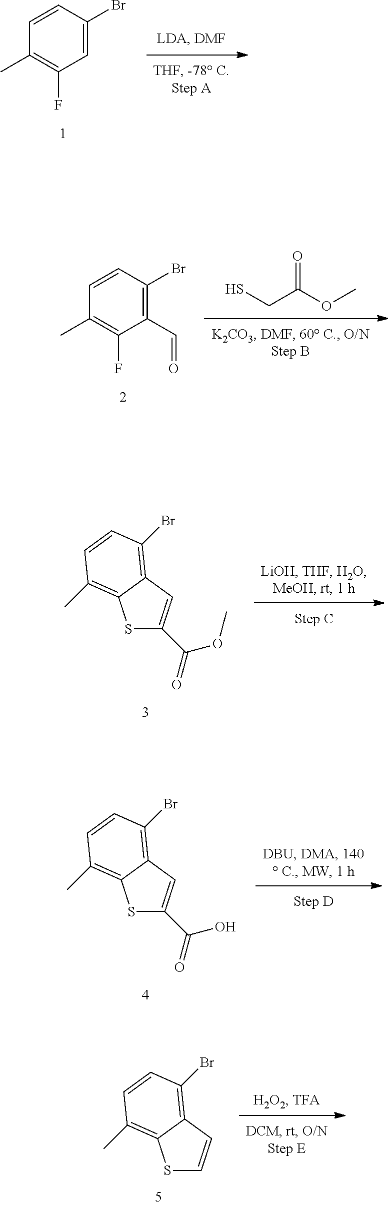

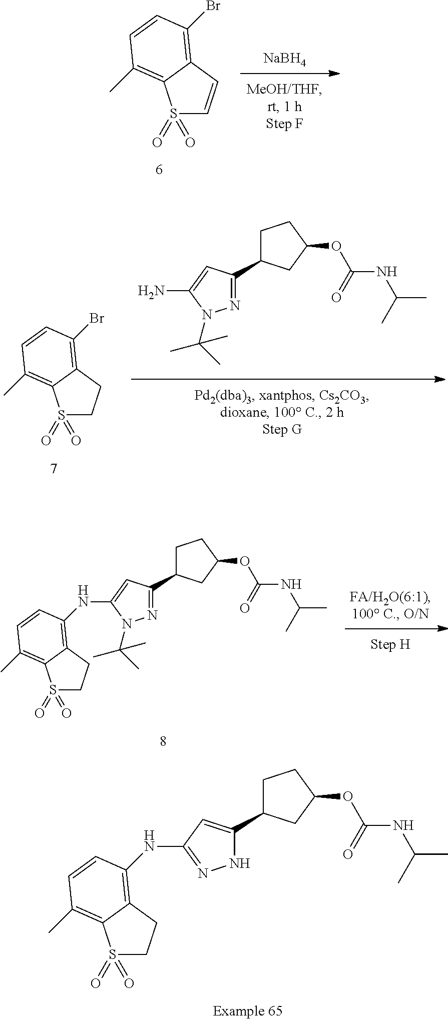

Step A: 6-bromo-2-fluoro-3-methylbenzaldehyde 4-bromo-2-fluoro-1-methylbenzene (3.33 mL, 26.4 mmol) was dissolved in THF (70 mL) and cooled to −78° C. LDA (3.50 mL, 26.4 mmol) was added dropwise at −78° C. After stirring at −78° C. for 30 min, DMF (2.05 mL, 26.4 mmol) was added at −78° C. After stirring at −78° C. for an additional 15 min, acetic acid (6 mL) and water (100 mL) was added and the mixture was warmed to room temperature. The reaction mixture was extracted with EA. The organic phase was washed with 1 M hydrochloride acid solution and brine, dried over magnesium sulfate and concentrated in vacuo afforded the title compound 6-bromo-2-fluoro-3-methylbenzaldehyde (5.00 g, 23.0 mmol, 87%) as a yellow oil. LCMS: ESI m/z 217.04/219.04 [M+H]⁺.

Step B: methyl 4-bromo-7-methylbenzo[b]thiophene-2-carboxylate To a solution of 6-bromo-2-fluoro-3-methylbenzaldehyde (2.50 g, 11.5 mmol) in DMF (25 mL) was added methyl sulfanylacetate (1.55 mL, 17.3 mmol) and K₂CO₃ (3.18 g, 23.0 mmol). The reaction mixture was stirred at 60° C. overnight. After completion by LCMS, the cooled reaction mixture was dissolved in EA (100 mL), washed with H₂O (50 mL) and brine, dried over sodium sulfate and concentrated to afford methyl methyl 4-bromo-7-methylbenzo[b]thiophene-2-carboxylate (3.00 g, 10.5 mmol, 91%) as a brown oil. LCMS: ESI m/z 285.15 [M+H]⁺.

Step C: 4-bromo-7-methylbenzo[b]thiophene-2-carboxylic acid To a solution of methyl 4-bromo-7-methylbenzo[b]thiophene-2-carboxylate (3.00 g, 10.5 mmol) and in THF: H₂O: MeOH (20 mL: 10 mL: 10 mL), LiOH (0.88 mL, 31.6 mmol) is added. The reaction mixture is continued stirring for 2 h. After completion, reaction mixture is poured on chilled 1 N aqueous hydrochloric acid, and extracted with ethyl acetate. The organic phase was washed with water and brine, dried over anhydrous sodium sulfate and concentrated to dryness under reduced pressure to afford 4-bromo-7-methylbenzo[b]thiophene-2-carboxylic acid (2.80 g, 10.3 mmol, 98%) as a brown oil. LCMS: ESI m/z 271.13 [M+H]⁺.

Step D: 4-bromo-7-methylbenzo[b]thiophene To a solution of 4-bromo-7-methylbenzo[b]thiophene-2-carboxylic acid (0.583 mL, 3.69 mmol) in DMA (10 mL) was added DBU (0.551 mL, 3.69 mmol) in a microwave vial. The sealed vial was irradiated in the microwave tube on a Biotage Smith Synthesizer at 140° C. for 1 hr. The reaction was complete detected by LCMS. The cooled reaction mixture was dissolved in EA (30 mL), washed with H₂O (20 mL) and brine, dried over sodium sulfate and concentrated. The residue was purified by silica gel chromatography (eluting with 0-100% EA in PE) to afford 4-bromo-7-methylbenzo[b]thiophene (500 mg, 2.20 mmol, 60%) as a white solid. No Ms. ¹H NMR (400 MHz, DMSO) δ 7.94 (brs, 1H), 7.59-7.53 (m, 1H), 7.48 (d, J=3.6 Hz, 1H), 7.17 (d, J=4.4 Hz, 1H), 2.50 (s, 3H).

Step E: 4-bromo-7-methylbenzo[b]thiophene 1,1-dioxide To a solution of 4-bromo-7-methylbenzo[b]thiophene (740 mg, 3.26 mmol) in DCM (5 mL) and TFA (5 mL) was added H₂O₂(4.5 mL, 44.1 mmol, 30% in H₂O). The reaction mixture was stirred at room temperature overnight. The reaction was complete detected by TLC. The cooled reaction mixture was dissolved in EA (50 mL), washed with H₂O (20 mL) and brine, dried over sodium sulfate and concentrated. The residue was purified by silica gel chromatography (eluting with 0-50% EA in PE) to afford 4-bromo-7-methylbenzo[b]thiophene 1,1-dioxide (720 mg, 2.78 mmol, 85.3%) as a white solid. LCMS: ESI m/z 259.12/261.12 [M+H]⁺. ¹H NMR (400 MHz, DMSO) δ 7.79 (d, J=8.0 Hz, 1H), 7.62-7.50 (m, 2H), 7.38 (d, J=8.0 Hz, 1H), 2.49 (s, 3H).

Step F: 4-bromo-7-methyl-2,3-dihydrobenzo[b]thiophene 1,1-dioxide To a solution of 4-bromo-7-methylbenzo[b]thiophene 1,1-dioxide (350 mg, 1.35 mmol) in MeOH (4 mL) and THF (4 mL) was added NaBH₄ (0.44 mL, 13.5 mmol) at 0° C. The reaction mixture was stirred at room temperature for 1 hr. The reaction was complete detected by TLC. The reaction mixture was dissolved in EA (10 mL), washed with H₂O (10 mL) and brine, dried over sodium sulfate and concentrated. The residue was purified by silica gel chromatography (eluting with 0-100% EA in PE) to afford 4-bromo-7-methyl-2,3-dihydrobenzo[b]thiophene 1,1-dioxide (250 mg, 0.96 mmol, 71%) as a white solid. No Ms. ¹H NMR (400 MHz, DMSO) δ 7.81 (d, J=7.6 Hz, 1H), 7.29 (d, J=7.6 Hz, 1H), 3.66 (brs, 2H), 3.23 (brs, 2H), 2.47 (s, 3H).

Step G: (1R,3S)-3-(1-(tert-butyl)-5-((7-methyl-1,1-dioxido-2,3-dihydrobenzo[b]thiophen-4-yl)amino)-1H-pyrazol-3-yl)cyclopentyl isopropylcarbamate To a solution of 4-bromo-7-methyl-2,3-dihydrobenzo[b]thiophene 1,1-dioxide (150 mg, 0.57 mmol) in dioxane (10 mL) was added (1R,3S)-3-(5-amino-1-(tert-butyl)-1H-pyrazol-3-yl)cyclopentyl isopropylcarbamate (150 mg, 0.486 mmol), Cs₂CO₃ (561 mg, 1.72 mmol), Pd₂(dba)₃ (52.6 mg, 0.057 mmol) and xantphos (66 mg, 0.12 mmol). The reaction mixture was stirred at 100° C. under N₂ for 2 hrs. The reaction was complete detected by LCMS. The cooled reaction mixture was dissolved in EA (50 mL), washed with H₂O (20 mL) and brine, dried over sodium sulfate and concentrated. The residue was purified by silica gel chromatography (eluting with 20-100% EA in PE) to afford (1R,3S)-3-(1-(tert-butyl)-5-((7-methyl-1,1-dioxido-2,3-dihydrobenzo[b]thiophen-4-yl)amino)-1H-pyrazol-3-yl)cyclopentyl isopropylcarbamate (230 mg, 0.471 mmol, 81.9%) as a yellow solid. LCMS: ESI m/z 489.65 [M+H]⁺.

Step G: (1R,3S)-3-(3-((7-methyl-1,1-dioxido-2,3-dihydrobenzo[b]thiophen-4-yl)amino)-1H-pyrazol-5-yl)cyclopentyl isopropylcarbamate A solution of (1R,3S)-3-(1-(tert-butyl)-5-((7-methyl-1,1-dioxido-2,3-dihydrobenzo[b]thiophen-4-yl)amino)-1H-pyrazol-3-yl)cyclopentyl isopropylcarbamate (230 mg, 0.47 mmol) in formic acid (6 mL) was stirred at 100° C. overnight. LCMS showed the reaction was completed. The reaction mixture was concentrated and purified by silica gel chromatography (eluting with 0-12% MeOH in DCM) and prep-HPLC (C18, 30˜ 95% ACN in H₂O with 0.1% TFA) to afford (1R,3S)-3-(3-((7-methyl-1,1-dioxido-2,3-dihydrobenzo[b]thiophen-4-yl)amino)-1H-pyrazol-5-yl)cyclopentyl isopropylcarbamate (122 mg, 0.28 mmol, 60%) as a white solid. LCMS: ESI m/z 433.54 [M+H]⁺. ¹H NMR (400 MHz, DMSO) δ 7.97-7.81 (m, 2H), 7.13 (d, J=7.6 Hz, 1H), 6.92 (s, 1H), 5.76 (s, 1H), 4.99 (s, 1H), 3.17 (s, 2H), 3.10-3.00 (m, 1H), 2.48-2.41 (m, 1H), 2.38 (s, 3H), 2.08-1.82 (m, 2H), 1.76-1.65 (m, 2H), 1.65-1.54 (m, 1H), 1.03 (brs, 6H).

### Example 66: Synthesis of (1R,3S)-3-(5-((7-fluoro-1,1-dioxido-2,3-dihydrobenzo[d]isothiazol-4-yl)amino)-1H-pyrazol-3-yl)cyclopentyl isopropylcarbamate

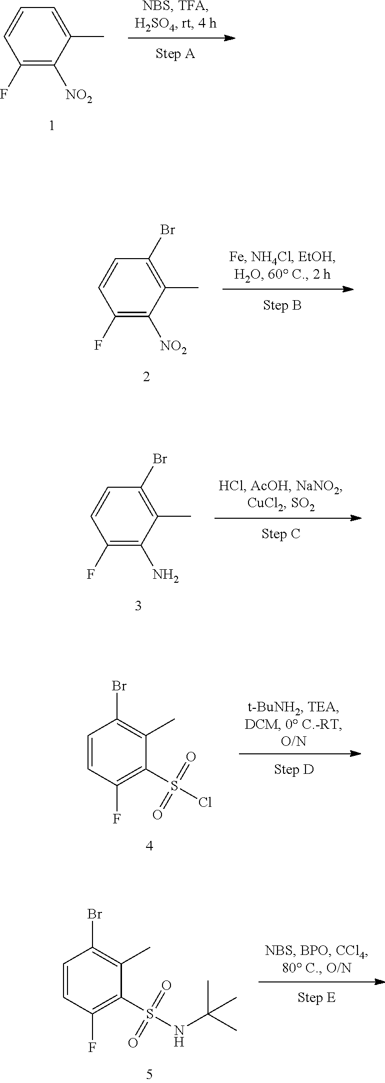

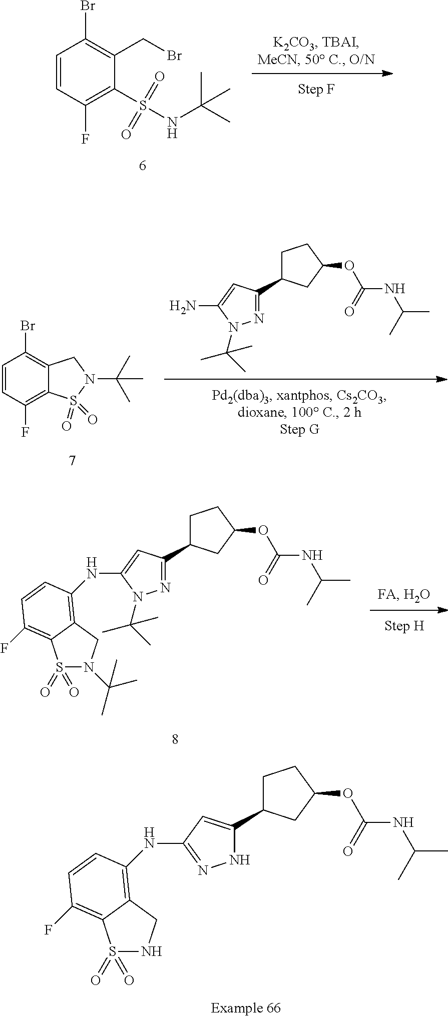

Step A. 4-bromo-1-fluoro-3-methyl-2-nitrobenzene. To a stirred solution of 1-fluoro-3-methyl-2-nitrobenzene (11 g, 67.7 mmol) in TFA (50 mL) was added con·H₂SO₄ (20 mL) and NBS (14.5 g, 81.2 mmol) slowly at 0° C. After stirred at rt for 4 h, the cooled mixture was poured into ice-water (100 mL) slowly and the precipitate that formed collected by filtration, washed with water and dried under vacuum to give 4-bromo-1-fluoro-3-methyl-2-nitrobenzene (14 g, 59.8 mmol, 88%) as a white solid. LCMS: m/z 235 [M+H]⁺.

Step B. 3-bromo-6-fluoro-2-methylaniline. To a stirred solution of 4-bromo-1-fluoro-3-methyl-2-nitrobenzene (14 g, 59.8 mmol) in EtOH (80 mL)/H₂O (80 mL) was added Fe (30.5 g, 547 mmol) and NH₄Cl (11 g, 205 mmol) at rt. After stirred at 60° C. for 3 h, the cooled mixture was filtered. The filtrate was poured into water (100 mL) and extracted with EtOAc (60 mL*2). The combined organic phase was washed with brine, dried with Na₂SO₄, filtered and concentrated. The residue was purified by chromatography (silica gel, 0-30%, EtOAc in PE) to give 3-bromo-6-fluoro-2-methylaniline (10 g, 50 mol, 73%) as a yellow solid. LCMS: m/z 204 [M+H]⁺.

Step C. 3-bromo-6-fluoro-2-methylbenzenesulfonyl chloride. To a stirred solution of 3-bromo-6-fluoro-2-methylaniline (5.1 g, 25 mmol) in MeCN (100 mL) at 0° C. was added con·HCl (21 mL), AcOH (21 mL) and a solution of NaNO₂ (2.6 g, 37.5 mmol) in 6 mL of water slowly. After stirred at 0° C. for 10 min, the mixture was purged with SO₂ gas for 15 min and a solution of CuCl₂ (3 g, 30 mmol) in 10 mL water was added dropwise at 0° C. After stirring at rt overnight, the mixture was poured into water (100 mL) and extracted with EtOAc (60 mL*2). The combined organic phase was washed with brine, dried with Na₂SO₄, filtered and concentrated. The residue was purified by chromatography (silica gel, 0-30%, EtOAc in PE) to give 3-bromo-6-fluoro-2-methylbenzenesulfonyl chloride (3.1 g, 10.8 mmol, 43%) as a white solid. No MS

Step D. 3-bromo-6-fluoro-2-methyl-N-(2-methylprop-2-yl)benzenesulfonamide. To a stirred solution of 3-bromo-6-fluoro-2-methylbenzenesulfonyl chloride (3 g, 10.4 mmol) in DCM (30 mL) was added TEA (3.6 mL, 26 mmol) and 2-methylpropan-2-amine (2.2 mL, 21 mmol) at 0° C. After stirred at rt overnight, the mixture was poured into water (50 mL) and extracted with DCM (30 mL*2). The combined organic phase was washed with brine, dried with Na₂SO₄, filtered and concentrated. The residue was purified by chromatography (silica gel, 0-50%, EtOAc in PE) to give 3-bromo-6-fluoro-2-methyl-N-(2-methylprop-2-yl)benzenesulfonamide (1.5 g, 4.6 mmol, 44%) as a yellow solid. No MS.

Step E. 3-bromo-2-(bromomethyl)-6-fluoro-N-(2-methylprop-2-yl)benzenesulfonamide. To a stirred solution of 3-bromo-6-fluoro-2-methyl-N-(2-methylprop-2-yl)benzenesulfonamide (1.5 g, 4.6 mmol) in CCl₄ (15 mL) was added NBS (1.2 g, 6.9 mmol) and benzoyl peroxide (340 mg, 1.4 mmol) at rt. After stirring at 80° C. overnight, the cooled mixture was poured into water (30 mL) and extracted with EtOAc (20 mL*2). The combined organic phase was washed with brine, dried with Na₂SO₄, filtered and concentrated. The residue was purified by chromatography (silica gel, 0-50%, EtOAc in PE) to give 3-bromo-2-(bromomethyl)-6-fluoro-N-(2-methylprop-2-yl)benzenesulfonamide (1.6 g, 3.9 mmol, 86%) as a yellow solid. No MS.

Step F. 4-bromo-7-fluoro-2-(2-methylprop-2-yl)-2,3-dihydro-1λ<sup>6</sup>-benzo[2,1-d][1,2]thiazole-1,1-dione. To a stirred solution of 3-bromo-2-(bromomethyl)-6-fluoro-N-(2-methylprop-2-yl)benzenesulfonamide (600 mg, 1.5 mmol) in MeCN (20 mL) was added Cs₂CO₃ (1.2 mg, 3.7 mmol) and TBAI (165 mg, 0.45 mmol) at rt. After stirred at 50° C. for 3 h, the cooled mixture was filtered. The filtrate was poured into water (30 mL) and extracted with EtOAc (30 mL*2). The combined organic phase was washed with brine, dried with Na₂SO₄, filtered and concentrated. The residue was purified by chromatography (silica gel, 0-50%, EtOAc in PE) to give 4-bromo-7-fluoro-2-(2-methylprop-2-yl)-2,3-dihydro-1λ⁶-benzo[2,1-d][1,2]thiazole-1,1-dione (440 mg, 1.4 mmol, 92%) as a yellow solid. No MS.

Step G. (1R,3S)-3-(5-{[7-fluoro-2-(2-methylprop-2-yl)-1,1-dioxo-2,3-dihydro-1λ<sup>6</sup>-benzo[2,1-d][1,2]thiazol-4-yl]amino}-1-(2-methylprop-2-yl)pyrazol-3-yl)cyclopentyl (prop-2-ylamino)methanoate. To a stirred solution of 4-bromo-7-fluoro-2-(2-methylprop-2-yl)-2,3-dihydro-1⁶-benzo[2,1-d][1,2]thiazole-1,1-dione (60 mg, 0.18 mmol) in dioxane (6 mL) was added (1R,3S)-3-[5-amino-1-(2-methylprop-2-yl)pyrazol-3-yl]cyclopentyl (prop-2-ylamino)methanoate (50 mg, 0.16 mmol), Pd₂(dba)₃ (15 mg, 0.02 mmol), Xant-PHOS (18 mg, 0.03 mmol) and Cs₂CO₃ (105 mg, 0.32 mmol) at rt. After stirred at 100° C. for 2 h, the cooled mixture was filtered. The filtrate was poured into water (15 mL) and extracted with EtOAc (10 mL*2). The combined organic phase was washed with brine, dried with Na₂SO₄ and concentrated. The residue was purified by chromatography (silica gel, 0-60%, EtOAc in PE) to give crude (1R,3S)-3-(5-{[7-fluoro-2-(2-methylprop-2-yl)-1,1-dioxo-2,3-dihydro-1λ⁶-benzo[2,1-d][1,2]thiazol-4-yl]amino}-1-(2-methylprop-2-yl)pyrazol-3-yl)cyclopentyl (prop-2-ylamino)methanoate (50 mg, 0.09 mmol, 56%) as a yellow solid. LCMS: m/z 550 [M+H]⁺.

Step H. (1R,3S)-3-(5-((7-fluoro-1,1-dioxido-2,3-dihydrobenzo[d]isothiazol-4-yl)amino)-1H-pyrazol-3-yl)cyclopentyl isopropylcarbamate. To a stirred solution of (1R,3S)-3-(1-(tert-butyl)-5-((2-(tert-butyl)-7-fluoro-1,1-dioxido-2,3-dihydrobenzo[d]isothiazol-4-yl)amino)-1H-pyrazol-3-yl)cyclopentyl isopropylcarbamate (50 mg, 0.09 mmol) in HCOOH (4 mL) was added H₂O (0.5 mL, 55 mmol) at rt. After stirring at 100° C. overnight, the cooled mixture was concentrated. The residue was purified by prep-HPLC (C18, 40˜ 90% MeCN in H₂O with 0.1% TFA) to give (1R,3S)-3-(5-((7-fluoro-1,1-dioxido-2,3-dihydrobenzo[d]isothiazol-4-yl)amino)-1H-pyrazol-3-yl)cyclopentyl isopropylcarbamate (6 mg, 0.014 mmol, 15%) as a white solid. LCMS: m/z 438 [M+H]⁺. ¹H NMR (400 MHz, DMSO) δ 8.05-7.91 (m, 3H), 7.25 (dd, J=8.8 Hz, 8.8 Hz, 1H), 6.98-6.84 (m, 1H), 5.73 (s, 1H), 4.99 (s, 1H), 4.31 (d, J=4.4 Hz, 2H), 3.45-3.39 (m, 1H), 3.09-3.01 (m, 1H), 2.51-2.41 (m, 1H), 2.04-1.87 (m, 2H), 1.82-1.64 (m, 3H), 1.05-0.99 (m, 6H).

### Example 67: synthesis of (1R,3S)-3-{5-[(7-fluoro-2-methyl-1,1-dioxo-2,3-dihydro-1λ<sup>6</sup>-benzo[2,1-d][1,2]thiazol-4-yl)amino]-2H-pyrazol-3-yl}cyclopentyl (prop-2-ylamino)methanoate

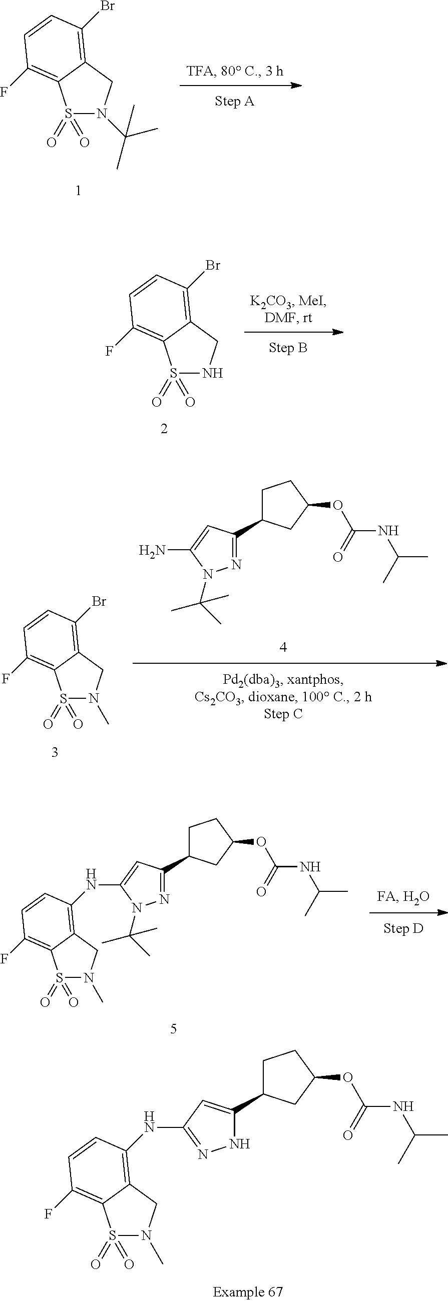

### Step A. 4-bromo-7-fluoro-2,3-dihydro-1λ<sup>6</sup>-benzo[2,1-d][1,2]thiazole-1,1-dione

A mixture of 4-bromo-7-fluoro-2-(2-methylprop-2-yl)-2,3-dihydro-1λ⁶-benzo[2,1-d][1,2]thiazole-1,1-dione (220 mg, 0.68 mmol) in TFA (8 mL) was stirred at 80° C. for 3 h. The reaction mixture was concentrated to give crude 4-bromo-7-fluoro-2,3-dihydro-1λ⁶-benzo[2,1-d][1,2]thiazole-1,1-dione (180 mg, 0.67 mmol, 99%) as a yellow solid. LCMS: m/z 267 (M+H)+.

Step B. 4-bromo-7-fluoro-2-methyl-2,3-dihydro-1λ<sup>6</sup>-benzo[2,1-d][1,2]thiazole-1,1-dione. To a stirred solution of 4-bromo-7-fluoro-2,3-dihydro-1λ⁶-benzo[2,1-d][1,2]thiazole-1,1-dione (180 mg, 0.67 mmol) in DMF (8 mL) was added K₂CO₃ (188 mg, 1.3 mmol) and iodomethane (145 mg, 1.1 mmol) at rt. After stirred at rt overnight, the mixture was poured into water (30 mL) and extracted with EtOAc (20 mL*2). The combined organic phase was washed with brine, dried with Na₂SO₄, filtered and concentrated. The residue was purified by chromatography (silica gel, 0-40%, EtOAc in PE) to give 4-bromo-7-fluoro-2-methyl-2,3-dihydro-1λ⁶-benzo[2,1-d][1,2]thiazole-1,1-dione (120 mg, 0.42 mmol, 63%) as a yellow solid. LCMS: m/z 281 [M+H]⁺.

Step C. (1R,3S)-3-{5-[(7-fluoro-2-methyl-1,1-dioxo-2,3-dihydro-1λ<sup>6</sup>-benzo[2,1-d][1,2]thiazol-4-yl)amino]-1-(2-methylprop-2-yl)pyrazol-3-yl}cyclopentyl (prop-2-ylamino)methanoate. To a stirred solution of 4-bromo-7-fluoro-2-methyl-2,3-dihydro-1λ⁶-benzo[2,1-d][1,2]thiazole-1,1-dione (120 mg, 0.42 mmol) in dioxane (8 mL) was added (1R,3S)-3-[5-amino-1-(2-methylprop-2-yl)pyrazol-3-yl]cyclopentyl (prop-2-ylamino)methanoate (80 mg, 0.26 mmol), Pd₂(dba)₃ (23 mg, 0.02 mmol), Xant-PHOS (30 mg, 0.05 mmol) and Cs₂CO₃ (84 mg, 0.26 mmol) at rt. After stirred at 100° C. under N₂ for 3 h, the cooled reaction mixture was filtered. The filtrate was poured into water (30 mL) and extracted with EtOAc (20 mL*2). The combined organic phase was washed with brine, dried with Na₂SO₄, filtered and concentrated. The residue was purified by chromatography (silica gel, 0-60%, EtOAc in PE) to give (1R,3S)-3-{5-[(7-fluoro-2-methyl-1,1-dioxo-2,3-dihydro-1λ⁶-benzo[2,1-d][1,2]thiazol-4-yl)amino]-1-(2-methylprop-2-yl)pyrazol-3-yl}cyclopentyl (prop-2-ylamino)methanoate (70 mg, 0.14 mmol, 53%) as a yellow solid. LCMS: m/z 631 [M+H]⁺.

Step D. (1R,3S)-3-{5-[(7-fluoro-2-methyl-1,1-dioxo-2,3-dihydro-1λ<sup>6</sup>-benzo[2,1-d][1,2]thiazol-4-yl)amino]-2H-pyrazol-3-yl}cyclopentyl (prop-2-ylamino)methanoate. To a stirred solution of (1R,3S)-3-{5-[(7-fluoro-2-methyl-1,1-dioxo-2,3-dihydro-1λ⁶-benzo[2,1-d][1,2]thiazol-4-yl)amino]-1-(2-methylprop-2-yl)pyrazol-3-yl}cyclopentyl (prop-2-ylamino)methanoate (70 mg, 0.14 mmol) in HCOOH (5 mL) was added H₂O (1 mL, 55 mmol) at rt. After stirred at 100° C. overnight, the cooled mixture was concentrated. The residue was purified by prep-HPLC (C18, 40˜ 90% MeCN in H₂O with 0.1% TFA) to give (1R,3S)-3-{5-[(7-fluoro-2-methyl-1,1-dioxo-2,3-dihydro-1λ⁶-benzo[2,1-d][1,2]thiazol-4-yl)amino]-2H-pyrazol-3-yl}cyclopentyl (prop-2-ylamino)methanoate (35 mg, 0.08 mmol, 56%) as a white solid. LCMS: m/z 452 [M+H]⁺. ¹H NMR (400 MHz, DMSO) δ 8.18-7.97 (m, 2H), 7.31 (t, J=8.4 Hz, 1H), 6.93 (brs, 1H), 5.73 (s, 1H), 4.99 (brs, 1H), 4.35 (s, 2H), 3.56 (brs, 1H), 3.06 (brs, 1H), 2.85 (s, 3H), 2.45-2.35 (m, 1H), 2.10-1.91 (m, 2H), 1.75-1.52 (m, 3H), 1.02 (brs, 6H).

### Example 68: synthesis of (1R,3S)-3-(3-((1,1-dioxido-2,3-dihydrothieno[3,2-c]pyridin-4-yl)amino)-1H-pyrazol-5-yl)cyclopentyl tert-butylcarbamate

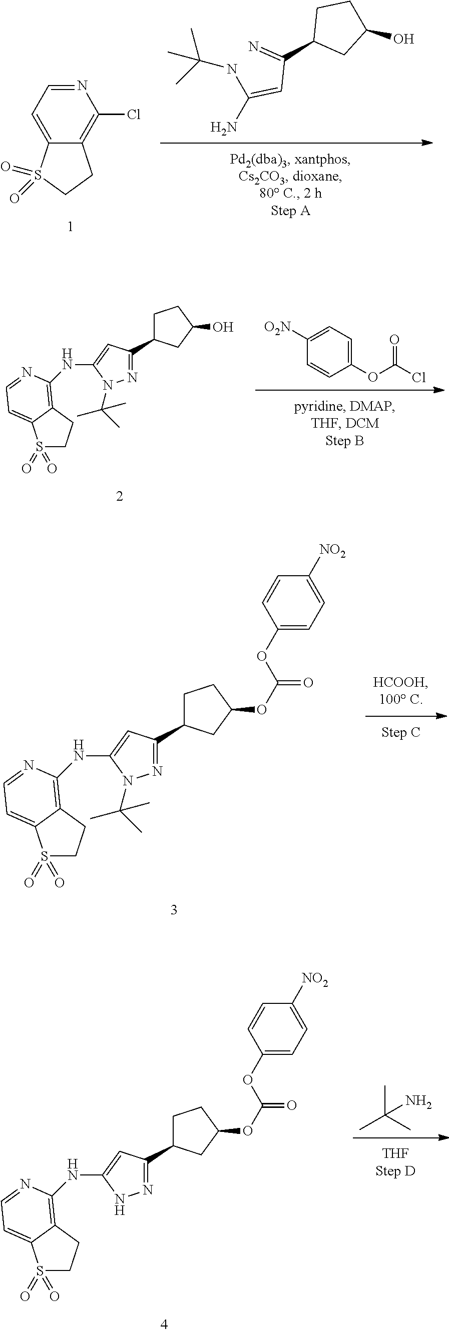

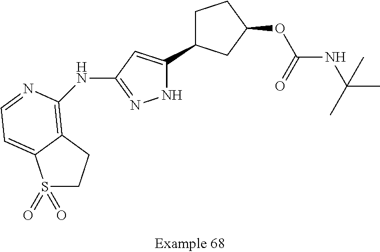 [SMILES: CC(C)(C)NC(=O)O[C@@H]1CC[C@H](c2cc(Nc3nccc4c3CCS4(=O)=O)n[nH]2)C1]

Step A: 4-({5-[(1S,3R)-3-hydroxycyclopentyl]-2-(2-methylprop-2-yl)pyrazol-3-yl}amino)-2,3-dihydro-1λ\hat{ }6-thieno[3,2-c]pyridine-1,1-dione To a stirred solution of 4-chloro-2,3-dihydro-1λ6-thieno[3,2-c]pyridine-1,1-dione (150 mg, 0.74 mmol), (1R,3S)-3-[5-amino-1-(2-methylprop-2-yl)pyrazol-3-yl]cyclopentan-1-ol (165 mg, 0.74 mmol), Cs₂CO₃ (720 mg, 2.21 mmol) and Xantphos (64 mg, 0.11 mmol) in 1,4-dioxane (8 mL) was added Pd₂(dba)₃ (67 mg, 0.074 mmol). The reaction was stirred at 80° C. under N₂ atmosphere for 4 h. TLC and LCMS indicated the reaction was over. The cooled reaction mixture was poured into water, extracted with EA. The organic layer was separated, washed with further brine, dried over Na₂SO₄ and concentrated. The residue was purified by silica gel column chromatography eluted with ethyl acetate in petroleum ether (gradient: 0˜ 80%) to afford the title compound 4-({5-[(1S,3R)-3-hydroxycyclopentyl]-2-(2-methylprop-2-yl)pyrazol-3-yl}amino)-2,3-dihydro-1λ6-thieno[3,2-c]pyridine-1,1-dione (60 mg, 0.15 mmol, 20.3%) as a yellow solid. LCMS: m/z 391[M+H]⁺.

Step B: (1R,3S)-3-(1-(tert-butyl)-5-((1,1-dioxido-2,3-dihydrothieno[3,2-c]pyridin-4-yl)amino)-1H-pyrazol-3-yl)cyclopentyl (4-nitrophenyl) carbonate To a solution of 4-({5-[(1S,3R)-3-hydroxycyclopentyl]-2-(2-methylprop-2-yl)pyrazol-3-yl}amino)-2,3-dihydro-1λ6-thieno[3,2-c]pyridine-1,1-dione (60 mg, 0.15 mmol), pyridine (36 mg, 0.46 mmol) and DMAP (2 mg, 0.015 mmol) in THF (3 mL) and DCM (3 mL) was added 4-Nitrophenyl chloroformate (37 mg, 0.18 mmol). The reaction was stirred at 60° C. for 18 hr. TLC and LCMS indicated the reaction was over. The cooled reaction mixture was poured into water, extracted with DCM. The organic layer was washed with further brine, dried over Na₂SO₄ and concentrated. The residue was purified by silica gel column chromatography eluted with ethyl acetate in petroleum ether (gradient: 0˜ 80%). The organic layer was collected, concentrated in vacuo, and dried to afford the title compound (1R,3S)-3-(1-(tert-butyl)-5-((1,1-dioxido-2,3-dihydrothieno[3,2-c]pyridin-4-yl)amino)-1H-pyrazol-3-yl)cyclopentyl (4-nitrophenyl) carbonate (40 mg, 0.072 mmol, 47%) as a yellow solid. LCMS: m/z 556 [M+H]⁺.

Step C: (1R,3S)-3-(5-((1,1-dioxido-2,3-dihydrothieno[3,2-c]pyridin-4-yl)amino)-1H-pyrazol-3-yl)cyclopentyl (4-nitrophenyl) carbonate A solution of (1R,3S)-3-(1-(tert-butyl)-5-((1,1-dioxido-2,3-dihydrothieno[3,2-c]pyridin-4-yl)amino)-1H-pyrazol-3-yl)cyclopentyl (4-nitrophenyl) carbonate (40 mg, 0.072 mmol) in HCOOH (5 mL) and H₂O (0.5 mL) was stirred at 100° C. for 18 h. TLC and LCMS indicated the reaction was over. The cooled reaction solution was concentrated to afford a crude title compound (1R,3S)-3-(5-((1,1-dioxido-2,3-dihydrothieno[3,2-c]pyridin-4-yl)amino)-1H-pyrazol-3-yl)cyclopentyl (4-nitrophenyl) carbonate (40 mg) as a yellow solid which was used directly for next step without purification. LCMS: m/z 550 [M+H]⁺.

Step D: (1R,3S)-3-(3-((1,1-dioxido-2,3-dihydrothieno[3,2-c]pyridin-4-yl)amino)-1H-pyrazol-5-yl)cyclopentyl tert-butylcarbamate A solution of (1R,3S)-3-(5-((1,1-dioxido-2,3-dihydrothieno[3,2-c]pyridin-4-yl)amino)-1H-pyrazol-3-yl)cyclopentyl (4-nitrophenyl) carbonate (50 mg, 0.1 mmol) and tert-butylamine (36 mg, 0.5 mmol) in THF (2 mL) was stirred at room temperature for 2 h. TLC and LCMS indicated the reaction was over. The reaction solution was concentrated. The residue was purified by prep-HPLC to give (1R,3S)-3-(3-((1,1-dioxido-2,3-dihydrothieno[3,2-c]pyridin-4-yl)amino)-1H-pyrazol-5-yl)cyclopentyl tert-butylcarbamate (3 mg, 0.007 mmol, 7%) as a white solid. LCMS: m/z 550 [M+H]⁺. ¹H NMR (400 MHz, DMSO): δ 9.38 (brs, 1H), 8.29 (d, J=5.2 Hz, 1H), 7.06 (d, J=5.2 Hz, 1H), 6.77 (s, 1H), 6.39 (s, 1H), 5.00 (brs, 1H), 3.70-3.61 (m, 2H), 3.30-3.26 (m, 2H), 3.12-3.05 (m, 1H), 2.52-2,43 (m, 1H), 2.07-2.00 (m, 1H), 1.94-1.87 (m, 1H), 1.81-1.71 (m, 2H), 1.70-1.61 (m, 1H), 1.21 (s, 9H).

### Example 69: synthesis of (1s,4s)-4-{5-[(1,1-dioxo-2,3-dihydro-1λ\hat{ }6-thieno[3,2-c]pyridin-4-yl)amino]-1H-pyrazol-3-yl}cyclohexyl (prop-2-ylamino)methanoate

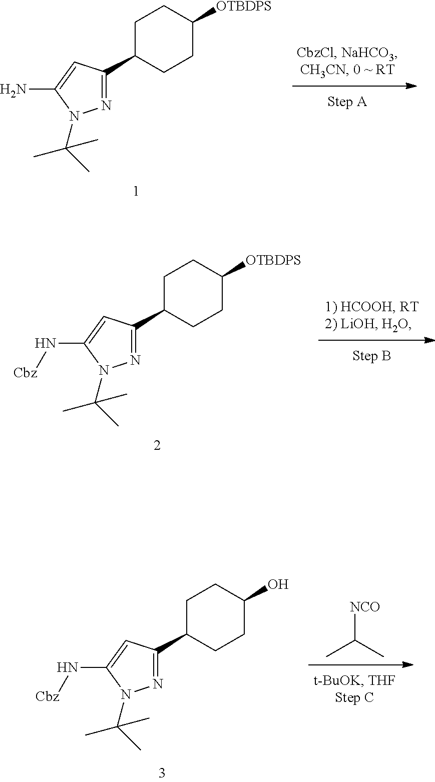

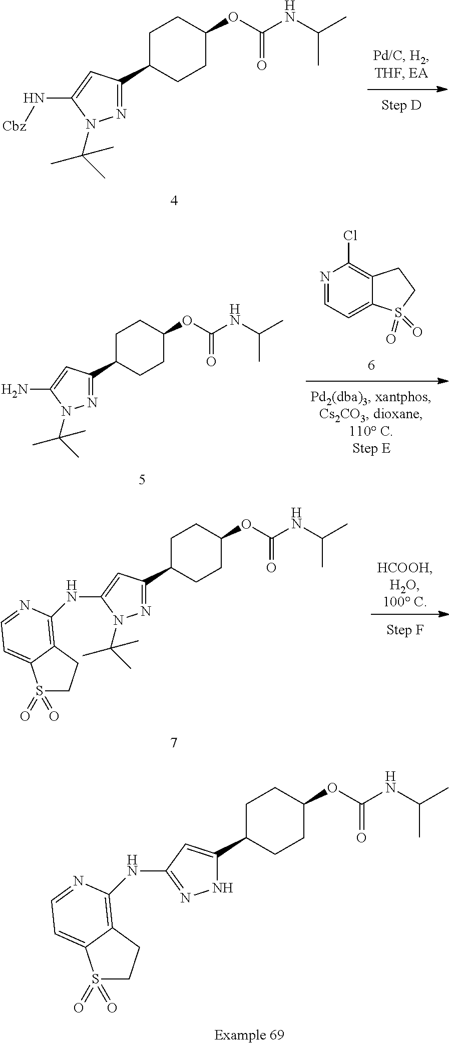 [SMILES: CC(C)NC(=O)O[C@H]1CC[C@@H](c2cc(NC(=O)OCc3ccccc3)n(C(C)(C)C)n2)CC1 ; CC(C)NC(=O)O[C@H]1CC[C@@H](c2cc(N)n(C(C)(C)C)n2)CC1 ; O=S1(=O)CCc2c1ccnc2Cl ; CC(C)NC(=O)O[C@H]1CC[C@@H](c2cc(Nc3nccc4c3CCS4(=O)=O)n(C(C)(C)C)n2)CC1 ; "HCOOH,H2O,100° C." ; CC(C)NC(=O)O[C@H]1CC[C@@H](c2cc(Nc3nccc4c3CCS4(=O)=O)n[nH]2)CC1]

Step A: benzyl (1-(tert-butyl)-3-((1s,4s)-4-((tert-butyldiphenylsilyl)oxy)cyclohexyl)-1H-pyrazol-5-yl)carbamate To a suspension of NaHCO₃(530 mg, 6.3 mmol) and 1-(tert-butyl)-3-((1s,4s)-4-((tert-butyldiphenylsilyl)oxy)cyclohexyl)-1H-pyrazol-5-amine (1.5 g, 3.15 mmol) in CH₃CN (30 mL) was added benzyl chloromethanoate (700 mg, 4.1 mmol) at 0° C. dropwise. The result mixture was stirred at room temperature for 18 h. TLC and LCMS indicated the reaction was over. The mixture was poured into water, extracted with EA. The organic layer was separated, washed with further brine, dried over Na₂SO₄, filtered and concentrated. The residue was purified by silica gel column chromatography eluted with ethyl acetate in petroleum ether (gradient: 0˜ 40%) to afford the title compound to afford the title compound benzyl (1-(tert-butyl)-3-((1s,4s)-4-((tert-butyldiphenylsilyl)oxy)cyclohexyl)-1H-pyrazol-5-yl)carbamate (1.5 g, 2.5 mmol, 78%) as a colorless oil. LCMS: m/z 610 [M+H]⁺. ¹H NMR (400 MHz, DMSO) δ 9.07 (s, 1H), 7.62 (dd, J=7.2, 1.6 Hz, 4H), 7.49-7.25 (m, 11H), 5.90 (s, 1H), 5.18-5.10 (m, 2H), 4.50 (d, J=5.6 Hz, 1H), 4.08-3.99 (m, 1H), 1.94-1.82 (m, 2H), 1.73-1.55 (m, 4H), 1.50-1.38 (m, 11H), 1.04 (s, 9H).

Step B: benzyl (1-(tert-butyl)-3-((1s,4s)-4-hydroxycyclohexyl)-1H-pyrazol-5-yl)carbamate A solution of benzyl (1-(tert-butyl)-3-((1s,4s)-4-((tert-butyldiphenylsilyl)oxy)cyclohexyl)-1H-pyrazol-5-yl)carbamate (1.5 g, 2.5 mmol) in HCOOH (15 mL) was stirred at 50° C. for 5 h. TLC and LCMS indicated the reaction was over. The reaction solution was concentrated, and the residue was dissolved in MeOH (10 mL) and H₂O (10 mL). LiOH (512 mg, 12.5 mmol) was added and the following mixture was stirred at room temperature for another 1 h. The reaction mixture was diluted with H₂O, extracted with EA. The organic layer was separated, washed with brine, dried with Na₂SO₄, filtered and concentrated. The residue was purified by a silica gel to give benzyl (1-(tert-butyl)-3-((1s,4s)-4-hydroxycyclohexyl)-1H-pyrazol-5-yl)carbamate (560 mg, 1.5 mmol, 61.3%) as a yellow solid. LCMS: m/z 372 [M+H]⁺. ¹H NMR (400 MHz, DMSO) δ 9.04 (s, 1H), 7.47-7.31 (m, 5H), 5.89 (s, 1H), 5.12 (s, 2H), 4.29 (d, J=3.2 Hz, 1H), 3.77 (s, 1H), 2.52-2.44 (m, 1H), 1.85-1.73 (m, 2H), 1.65-1.50 (m, 6H), 1.48 (s, 9H).

Step C: benzyl (1-(tert-butyl)-3-((1s,4s)-4-((isopropylcarbamoyl)oxy)cyclohexyl)-1H-pyrazol-5-yl)carbamate To a solution of benzyl (1-(tert-butyl)-3-((1s,4s)-4-hydroxycyclohexyl)-1H-pyrazol-5-yl)carbamate (500 mg, 1.3 mmol) in THF (15 mL) was added t-BuOK (227 mg, 2.0 mmol) by portions at 0° C. and stirred at 0° C. for 30 min. 1-oxo-N-(prop-2-yl)methanimine (172 mg, 2.0 mmol) in THF (4 ml) was added dropwise and the result solution was stirred at room temperature for 1 h. The reaction mixture poured into ice-water, extracted with EA. The organic layer was separated, washed with brine, dried with Na₂SO₄, filtered and concentrated. The residue was purified by a silica gel to give benzyl (1-(tert-butyl)-3-((1s,4s)-4-((isopropylcarbamoyl)oxy)cyclohexyl)-1H-pyrazol-5-yl)carbamate (500 mg, 1.1 mmol, 81%) as a yellow solid. LCMS: m/z 457 [M+H]⁺.

Step D: (1s,4s)-4-(5-amino-1-(tert-butyl)-1H-pyrazol-3-yl)cyclohexyl isopropylcarbamate A suspension of benzyl (1-(tert-butyl)-3-((1s,4s)-4-((isopropylcarbamoyl)oxy)cyclohexyl)-1H-pyrazol-5-yl)carbamate (500 mg, 1.1 mmol) and 10% Pd/C (100 mg) in THF (5 mL) and EA (5 mL) was stirred at room temperature under H₂ atmosphere for 18 h. TLC and LCMS indicated the reaction was over. The reaction mixture filtered and the filtrate was concentrated to give (1s,4s)-4-(5-amino-1-(tert-butyl)-1H-pyrazol-3-yl)cyclohexyl isopropylcarbamate (323 mg, 1.1 mmol, 100%) as a yellow solid used directly for next step. LCMS: m/z 323 [M+H]⁺

Step E: (1s,4s)-4-(1-(tert-butyl)-5-((1,1-dioxido-2,3-dihydrothieno[3,2-c]pyridin-4-yl)amino)-1H-pyrazol-3-yl)cyclohexyl isopropylcarbamate To a stirred solution of (1s,4s)-4-(5-amino-1-(tert-butyl)-1H-pyrazol-3-yl)cyclohexyl isopropylcarbamate (79 mg, 0.25 mmol), 4-chloro-2,3-dihydrothieno[3,2-c]pyridine 1,1-dioxide(51 mg, 0.25 mmol), Cs₂CO₃ (240 mg, 0.75 mmol) and Xantphos (14 mg, 0.025 mmol) in 1,4-dioxane (8 mL) was added Pd₂dba₃ (23 mg, 0.025 mmol). The result mixture was stirred at 100° C. under N₂ for 18 hr. TLC and LCMS indicated the reaction was over. The cooled reaction mixture was diluted with EA and water. The organic layer was separated, washed with brine, dried with Na₂SO₄, filtered and concentrated. The residue was purified by silica gel column chromatography eluted with ethyl acetate in petroleum ether (gradient: 0˜ 50%) to afford the title compound (1s,4s)-4-(1-(tert-butyl)-5-((1,1-dioxido-2,3-dihydrothieno[3,2-c]pyridin-4-yl)amino)-1H-pyrazol-3-yl)cyclohexyl isopropylcarbamate (50 mg, 0.10 mmol, 41%) as a yellow solid. LCMS: m/z 490 [M+H]⁺

Step F: (1s,4s)-4-(3-((1,1-dioxido-2,3-dihydrothieno[3,2-c]pyridin-4-yl)amino)-1H-pyrazol-5-yl)cyclohexyl isopropylcarbamate A solution of (1s,4s)-4-(1-(tert-butyl)-5-((1,1-dioxido-2,3-dihydrothieno[3,2-c]pyridin-4-yl)amino)-1H-pyrazol-3-yl)cyclohexyl isopropylcarbamate (50 mg, 0.1 mmol) in HCOOH (4 mL) and H₂O (0.4 mL) was stirred at 100° C. for 18 h. TLC and LCMS indicated the reaction was over. The reaction mixture was concentrated and the residue was purified by prep-HPLC to give the desire product (1s,4s)-4-{5-[(1,1-dioxo-2,3-dihydro-16-thieno[3,2-c]pyridin-4-yl)amino]-1H-pyrazol-3-yl}cyclohexyl (prop-2-ylamino)methanoate (20 mg, 0.046 mmol, 45%) as a white solid. LCMS m/z: 434 [M+H]⁺. 1H NMR (400 MHz, DMSO) δ 9.61 (s, 1H), 8.31 (d, J=5.2 Hz, 1H), 7.11 (d, J=5.2 Hz, 1H), 6.92 (d, J=7.6 Hz, 1H), 6.36 (s, 1H), 4.77 (s, 1H), 3.70-3.65 (m, 2H), 3.64-3.53 (m, 1H), 3.29 (t, J=6.4 Hz, 2H), 2.78-2.70 (m, 1H), 1.86-1.61 (m, 8H), 1.05 (d, J=6.4 Hz, 6H).

### Example 70: synthesis of (1s,3s)-3-(3-((1,1-dioxido-2,3-dihydrothieno[3,2-c]pyridin-4-yl)amino)-1H-pyrazol-5-yl)cyclobutyl isopropylcarbamate

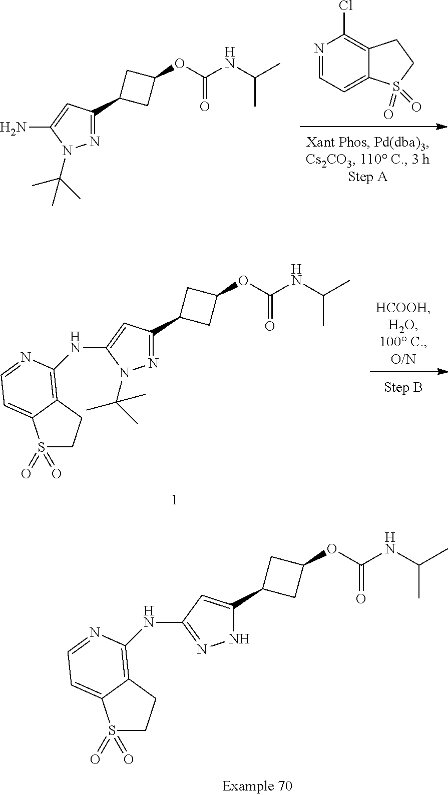

Step A: (1s,3s)-3-(1-(tert-butyl)-5-((1,1-dioxido-2,3-dihydrothieno[3,2-c]pyridin-4-yl)amino)-1H-pyrazol-3-yl)cyclobutyl isopropylcarbamate To a solution of (1s,3s)-3-[5-amino-1-(2-methylprop-2-yl)pyrazol-3-yl]cyclobutyl (prop-2-ylamino)methanoate (50.00 mg, 0.170 mmol) in dioxane (3 mL) was added 4-chloro-2,3-dihydro-1⁶-thieno[3,2-c]pyridine-1,1-dione (34.6 mg, 0.170 mmol), Xant Phos (9.83 mg, 0.017 mmol), Pd₂(dba)₃ (15.6 mg, 0.017 mmol) and Cs₂CO₃ (166 mg, 0.510 mmol). The reaction mixture was stirred at 110° C. for 4 h. TLC (PE/EA=1:1) showed starting material was consumed and new spot was observed. The cooled reaction mixture was diluted with water 50 mL, extracted with EA (50 mL×3). The organic phase was washed with brine, dried over Na₂SO₄ and concentrated. The residue was purified by silica gel chromatography (15 g column) using 0-50% EtOAc/hexane to afford (1s,3s)-3-(1-(tert-butyl)-5-((1,1-dioxido-2,3-dihydrothieno[3,2-c]pyridin-4-yl)amino)-1H-pyrazol-3-yl)cyclobutyl isopropylcarbamate (78.0 mg, 0.170 mmol, 100%) as a black solid. LCMS: 462 [M+H]⁺

Step B: (1s,3s)-3-(3-((1,1-dioxido-2,3-dihydrothieno[3,2-c]pyridin-4-yl)amino)-1H-pyrazol-5-yl)cyclobutyl isopropylcarbamate A solution of (1s,3s)-3-(1-(tert-butyl)-5-((1,1-dioxido-2,3-dihydrothieno[3,2-c]pyridin-4-yl)amino)-1H-pyrazol-3-yl)cyclobutyl isopropylcarbamate (60.0 mg, 0.130 mmol) in formic acid (3 mL) and H₂O (0.1 mL) was stirred at 100° C. overnight. LCMS and TLC showed the starting material was consumed completely. The residue was purified by prep-HPLC (C18, 0-70% acetonitrile in H₂O) to afford the title compound (1s,3s)-3-(3-((1,1-dioxido-2,3-dihydrothieno[3,2-c]pyridin-4-yl)amino)-1H-pyrazol-5-yl)cyclobutyl isopropylcarbamate (5 mg, 0.012 mmol, 9%) as a white solid. LCMS: 406 [M+H]⁺. ¹H NMR (400 MHz, DMSO) δ 12.27 (brs, 1H), 9.22 (s, 1H), 8.28 (d, J=5.2 Hz, 1H), 7.12-6.92 (m, 2H), 6.45 (s, 1H), 4.87-4.74 (m, 1H), 3.69-3.57 (m, 3H), 3.28-3.22 (m, 2H), 3.15-2.98 (m, 1H), 2.67 (brs, 2H), 2.15-2.03 (m, 2H), 1.04 (d, J=6.4 Hz, 6H).

### Example 71: Synthesis of (1R,3S)-3-{5-[(1,1-dioxo-2,3-dihydro-1λ<sup>6</sup>-thieno[3,2-c]pyridin-4-yl)amino]-2H-pyrazol-3-yl}cyclopentyl (cyclopropylamino)methanoate

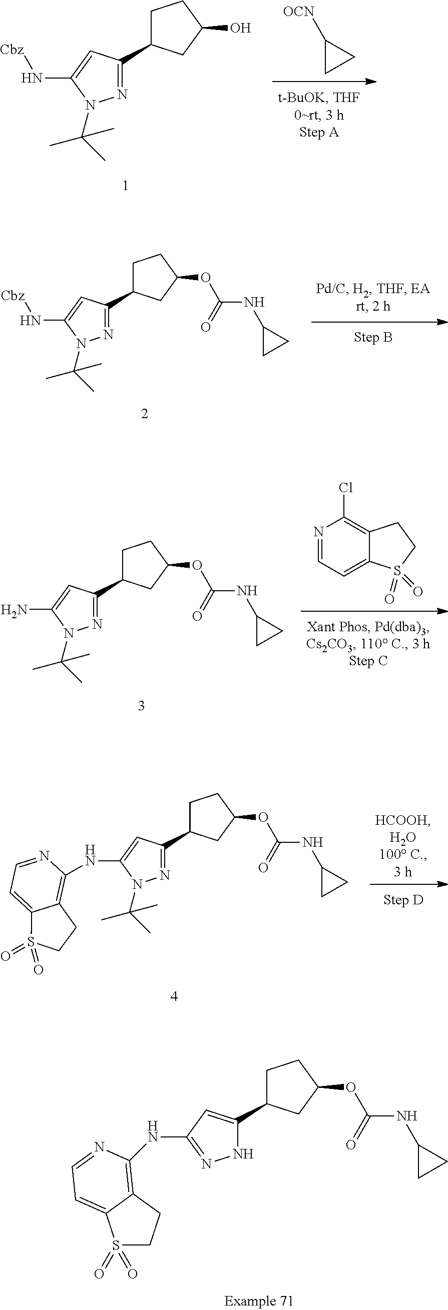

Step A: benzyl ({5-[(1S,3R)-3-{[(cyclopropylamino)carbonyl]oxy}cyclopentyl]-2-(2-methylprop-2-yl)pyrazol-3-yl}amino)methanoate To a solution of benzyl ({5-[(1S,3R)-3-hydroxycyclopentyl]-2-(2-methylprop-2-yl)pyrazol-3-yl}amino)methanoate (550 mg, 1.54 mmol) in THF (5 mL) was added potassium 2-methylpropan-2-olate (260 mg, 2.31 mmol) at 0° C. The reaction mixture was stirred at rt for 30 min, isocyanatocyclopropane (192 mg, 2.31 mmol) was added. The following mixture was stirred at 20° C. for 2 h. TLC (PE/EA=1:1) showed starting material was consumed and new spot was observed. The reaction mixture was diluted with water 50 mL, extracted with EA (50 mL×3). The organic phase was washed with brine, dried over Na₂SO₄ and concentrated. The residue was purified by silica gel chromatography (15 g column) using 0-50% EtOAc/hexane to afford benzyl ({5-[(1S,3R)-3-{[(cyclopropylamino)carbonyl]oxy}cyclopentyl]-2-(2-methylprop-2-yl)pyrazol-3-yl}amino)methanoate (500 mg, 1.14 mmol, 74%) as a yellow oil. LCMS: 441 [M+H]⁺. ¹H NMR (400 MHz, DMSO) δ 9.07 (s, 1H), 7.45-7.30 (m, 5H), 7.21 (s, 1H), 5.93 (d, J=5.2 Hz, 1H), 5.11 (s, 2H), 4.99 (s, 1H), 3.01-2.91 (m, 1H), 2.57-2.47 (m, 2H), 2.48-2.30 (m, 2H), 1.97-1.53 (m, 6H), 1.47 (s, 10H), 0.96-0.86 (m, 1H), 0.72-0.61 (m, 1H), 0.59-0.48 (m, 2H), 0.40-0.31 (m, 2H).

Step B: (1R,3S)-3-[5-amino-1-(2-methylprop-2-yl)pyrazol-3-yl]cyclopentyl (cyclopropylamino)methanoate To a solution of benzyl ({5-[(1S,3R)-3-{[(cyclopropylamino)carbonyl]oxy}cyclopentyl]-2-(2-methylprop-2-yl)pyrazol-3-yl}amino)methanoate (240 mg, 0.545 mmol) in THF (1.5 mL) and EA (1.5 mL) was added Pd/C (58.0 mg, 0.545 mmol) under H₂ balloon. The mixture was stirred at 20° C. for 2 h. LCMS showed starting material was consumed. The reaction mixture was diluted with 50 mL of water, extracted with EA (50 mL×3). The organic phase was washed with brine, dried over Na₂SO₄ and concentrated to afford (1R,3S)-3-[5-amino-1-(2-methylprop-2-yl)pyrazol-3-yl]cyclopentyl (cyclopropylamino)methanoate (150 mg, 0.490 mmol, 89.8%) as a yellow oil. LCMS: 307 [M+H]⁺

Step C: (1R,3S)-3-{5-[(1,1-dioxo-2,3-dihydro-1λ<sup>6</sup>-thieno[3,2-c]pyridin-4-yl)amino]-1-(2-methylprop-2-yl)pyrazol-3-yl}cyclopentyl (cyclopropylamino)methanoate To a solution of (1R,3S)-3-[5-amino-1-(2-methylprop-2-yl)pyrazol-3-yl]cyclopentyl (cyclopropylamino)methanoate (113 mg, 0.368 mmol) in dioxane (3 mL) was added 4-chloro-2,3-dihydro-1⁶-thieno[3,2-c]pyridine-1,1-dione (50 mg, 0.246 mmol), tris(1,5-diphenylpenta-1,4-dien-3-one) palladium(0) (20.0 mg, 0.025 mmol), Xant Phos(15.0 mg, 0.025 mmol) and Cs₂CO₃ (240 mg, 0.737 mmol). The reaction mixture was stirred at 110° C. under N₂ for 3 h. TLC (PE/EA=1:1) showed starting material was consumed and new spot was observed. The cooled reaction mixture was diluted with water 50 mL, extracted with EA (50 mL×3). The organic phase was washed with brine, dried over Na₂SO₄ and concentrated. The residue was purified by silica gel chromatography (15 g column) using 0-100% EtOAc/hexane to afford (1R,3S)-3-{5-[(1,1-dioxo-2,3-dihydro-1λ⁶-thieno[3,2-c]pyridin-4-yl)amino]-1-(2-methylprop-2-yl)pyrazol-3-yl}cyclopentyl (cyclopropylamino)methanoate (60.0 mg, 0.127 mmol) as a black solid. LCMS: 474 [M+H]⁺

Step D: (1R,3S)-3-{5-[(1,1-dioxo-2,3-dihydro-1λ<sup>6</sup>-thieno[3,2-c]pyridin-4-yl)amino]-2H-pyrazol-3-yl}cyclopentyl (cyclopropylamino)methanoate A solution of (1R,3S)-3-{5-[(1,1-dioxo-2,3-dihydro-1λ⁶-thieno[3,2-c]pyridin-4-yl)amino]-1-(2-methylprop-2-yl)pyrazol-3-yl}cyclopentyl (cyclopropylamino)methanoate (50.0 mg, 0.106 mmol) in formic acid (3 mL) and H₂O (0.1 mL) was stirred at 100° C. for 3 h. LCMS and TLC showed the starting material was consumed completely. The residue was purified by prep-HPLC (C18, 0-70% acetonitrile in H₂O) to afford the title compound (1R,3S)-3-{5-[(1,1-dioxo-2,3-dihydro-1λ⁶-thieno[3,2-c]pyridin-4-yl)amino]-2H-pyrazol-3-yl}cyclopentyl (cyclopropylamino)methanoate (10.1 mg, 0.024 mmol, 22.91%) as a white solid. LCMS: 418 [M+H]⁺. ¹H NMR (400 MHz, DMSO) δ 12.31 (s, 1H), 9.17 (s, 1H), 8.26 (d, J=5.2 Hz, 1H), 8.14 (s, 1H), 7.24 (s, 1H), 7.00 (d, J=5.2 Hz, 1H), 6.40 (s, 1H), 5.02 (s, 1H), 3.63 (t, J=6.8 Hz, 2H), 3.27-3.23 (m, 2H), 3.07 (brs, 1H), 2.47-2.42 (m, 1H), 2.07-2.01 (m, 1H), 1.97-1.89 (m, 1H), 1.81-1.62 (m, 3H), 0.59-0.50 (m, 2H), 0.37 (d, J=2.4 Hz, 2H).

### Example 72: synthesis of (1R,3S)-3-{5-[(1,1-dioxo-2,3-dihydro-1λ\hat{ }6-thieno[3,2-c]pyridin-6-yl)amino]-1H-pyrazol-3-yl}cyclopentyl (prop-2-ylamino)methanoate

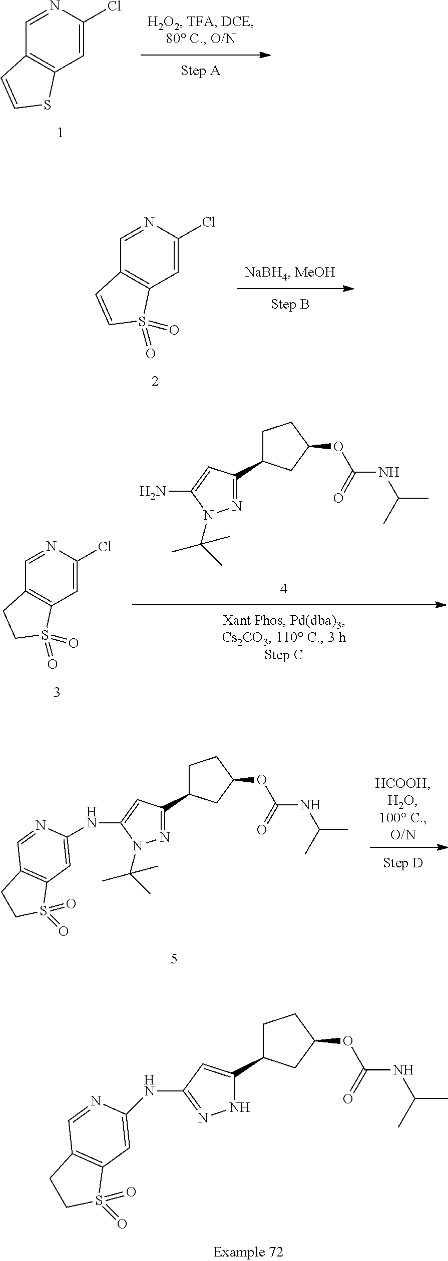

Step A: 6-chloro-1λ6-thieno[3,2-c]pyridine-1,1-dione To a solution of 6-chlorothieno[3,2-c]pyridine (2 g, 11.8 mmol) and TFA (2 mL) in DCE (150 mL) was added H₂O₂(2 mL, 19.6 mmol, 30% in H₂O) at 0° C. The reaction mixture was stirred at 80° C. for 18 hr. The cooled reaction mixture was poured into ice-water, quenched with NaS₂O₃, extracted with DCM. The organic layer was separated, washed with saturated NaHCO₃ solution and brine, dried over Na₂SO₄, filtered and concentrated. The residue was purified by silica gel column chromatography eluted with ethyl acetate in petroleum ether (gradient: 0˜ 30%) to afford 6-chloro-1λ6-thieno[3,2-c]pyridine-1,1-dione (100 mg, 0.5 mmol, 4%) as a yellow solid. LCMS: m/z 202 [M+H]⁺. ¹H NMR (400 MHz, DMSO) δ 8.75 (d, J=2.8 Hz, 1H), 8.36 (d, J=2.8 Hz, 1H), 7.88-7.81 (m, 1H), 7.68-7.60 (m, 1H).

Step B: 6-chloro-2,3-dihydro-1λ6-thieno[3,2-c]pyridine-1,1-dione To a solution of 6-chloro-1λ6-thieno[3,2-c]pyridine-1,1-dione (100 mg, 0.5 mmol) in MeOH (3 mL) and THF (3 mL) was added NaBH₄ (38 mg, 1.0 mmol) at 0° C. The reaction was stirred at room temperature for 30 min. The reaction was quenched with ice-water, extracted with EA. The organic layer was separated, washed with brine, dried over Na₂SO₄, filtered and concentrated. The residue was purified by silica gel column chromatography eluted with ethyl acetate in petroleum ether (gradient: 0˜ 40%) to afford 6-chloro-2,3-dihydro-1λ6-thieno[3,2-c]pyridine-1,1-dione (90 mg, 0.44 mmol, 89%) as a yellow solid. LCMS: m/z 204 [M+H]⁺. ¹H NMR (400 MHz, DMSO) δ 8.72 (s, 1H), 8.10 (s, 1H), 3.73 (t, J=6.8 Hz, 2H), 3.40 (t, J=6.8 Hz, 2H).

Step C: (1R,3S)-3-{5-[(1,1-dioxo-2,3-dihydro-1λ\hat{ }6-thieno[3,2-c]pyridin-6-yl)amino]-1-(2-methylprop-2-yl)pyrazol-3-yl}cyclopentyl (prop-2-ylamino)methanoate To a solution of 6-chloro-2,3-dihydro-16-thieno[3,2-c]pyridine-1,1-dione (50 mg, 0.25 mmol), Cs₂CO₃ (160 mg, 0.5 mmol), xantphos (14 mg, 0.025 mmol) and (1R,3S)-3-[5-amino-1-(2-methylprop-2-yl)pyrazol-3-yl]cyclopentyl (prop-2-ylamino)methanoate (68 mg, 0.22 mmol) in 1,4-dioxane (5 mL) was added Pd₂dba₃ (22 mg, 0.025 mmol). The reaction was stirred at 100° C. under N₂ atmosphere for 3 h. TLC and LCMS indicated the reaction was over. The cooled reaction mixture was diluted with EA and water. The organic layer was separated, washed with brine, dried over Na₂SO₄, filtered and concentrated. The residue was purified by silica gel column chromatography eluted with ethyl acetate in petroleum ether (gradient: 0˜ 70%) to afford (1R,3S)-3-{5-[(1,1-dioxo-2,3-dihydro-1λ\hat{ }6-thieno[3,2-c]pyridin-6-yl)amino]-1-(2-methylprop-2-yl)pyrazol-3-yl}cyclopentyl (prop-2-ylamino)methanoate (80 mg, 0.17 mmol, 68%) as a yellow solid. LCMS: m/z 476 [M+H]⁺.

Step D: (1R,3S)-3-{5-[(1,1-dioxo-2,3-dihydro-1λ\hat{ }6-thieno[3,2-c]pyridin-6-yl)amino]-1H-pyrazol-3-yl}cyclopentyl (prop-2-ylamino)methanoate A solution of (1R,3S)-3-{5-[(1,1-dioxo-2,3-dihydro-16-thieno[3,2-c]pyridin-6-yl)aminol-1-(2-methylprop-2-yl)pyrazol-3-yl}cyclopentyl (prop-2-ylamino)methanoate (80 mg, 0.17 mmol) in HCOOH (4 mL) and H₂O (0.4 mL) was stirred at 100° C. for 18 h. TLC and LCMS indicated the reaction was over. The reaction mixture was concentrated and the residue was purified by prep-HPLC to give the desire product (1R,3S)-3-{5-[(1,1-dioxo-2,3-dihydro-16-thieno[3,2-c]pyridin-6-yl)aminol-1H-pyrazol-3-yl}cyclopentyl (prop-2-ylamino)methanoate (45 mg, 0.11 mmol, 64%) LCMS m/z: 420 [M+H]⁺. ¹H NMR (400 MHz, DMSO) δ 9.80 (s, 1H), 8.38 (s, 1H), 7.71 (s, 1H), 6.95 (d, J=7.2 Hz, 1H), 5.98 (s, 1H), 5.03-4.98 (m, 1H), 3.67-3.50 (m, 3H), 3.26 (t, J=6.8 Hz, 2H), 3.14-3.00 (m, 1H), 2.49-2.42 (m, 1H), 2.11-1.99 (m, 1H), 1.97-1.85 (m, 1H), 1.78-1.56 (m, 3H), 1.03 (d, J=6.4 Hz, 6H).

### Biological Studies

### In Vitro Kinase Caliper Assays: CDK2/CycA2, CDK2/CycE1, CDK1/cyclinB and GSK3β

1× Kinase buffer (50 mM HEPES pH 7.5, 10 mM MgCl₂, 2 mM DTT and 0.01% Brij-35) The test compounds were prepared as 10 mM stock solution in 100% DMSO. The stock solution was then serially diluted 3-fold in 100% DMSO to 10 concentrations. 200 nL of each diluted compound solution was transferred to 384-well plate in duplicate. To each well, 10 μL of enzyme solution containing either CDK2/CycA2, CDK2/CycE1, CDK1/cyclinB, or GSK3β (final concentrations 10 nM, 3 nM, 1 nM, 1 nM respectively) in 1× Kinase buffer and the mix incubated at room temperature for 10 min. To initiate each reaction, 10 μL of peptide solution containing FAM-labelled peptide with final concentration at 3000 nM (FAM-P18 (5-FAM-QSPKKG-CONH2) for CDK2/CycA2, CDK2/CycE1, CDK1/cyclinB proteins, FAM-P15 (5-FAM-KRREILSRRPpSYR—COOH) for GSK30 protein) and ATP (final concentrations 30 μM, 77 μM or 20 μM, respectively) in 1× Kinase buffer was added to each well. All reactions were incubated at 28° C. for 30 min and then terminated by the addition of 30 μL stop buffer (100 mM HEPES pH 7.5, 50 mM EDTA, 0.2% Coating Reagent #3 and 0.015% Brij-35). All samples were then subjected to analysis using Caliper EZ Reader to read conversion values. IC50 values were then calculated by plotting dose-response curves and then using the XLfit application in Excel software.

TABLE 7
Biochemical Data (Caliper)

| | CDK1 | CDK2 | CDK2 | |
|---|---|---|---|---|
| | cyclinB | cyclinA2 | cyclinE1 | GSK3β |
| Example | activity assay: | activity assay: | activity assay: | activity assay: |
| Number | IC50 (nM) | IC50 (nM) | IC50 (nM) | IC50 (nM) |
| 1 | B | A | A | B |
| 2 | A | A | A | C |
| 3 | A | A | A | C |

In vitro kinase TR-FRET assays: CDK2/CycA2, CDK2/CycE1, CDK1/cyclinB and GSK3β

The test compounds were prepared as 10 mM stock solution in 100% DMSO. The stock solution was then serially diluted 3-fold in 100% DMSO to 10 concentrations. 200 nL of each diluted compound solution was transferred to 384-well plate in duplicate. To each well, 5 uL of enzyme and ATP mixture solution containing enzyme CDK2/CycA2, CDK2/CycE1, CDK1/cyclinB, or GSK30 (final concentrations 3 nM, 0.6 nM, 0.3 nM, 0.6 nM, respectively) and ATP (final concentrations 27 μM, 82 μM, 56 μM, 7.6 μM, respectively) in the assay buffer (50 mM HEPES, pH7.5, 10 mM MgCl₂, 1 mM DTT, 0.01% BSA, 0.01% Triton X-100). For negative control, 5 uL of assay buffer was added instead. The plate was incubated at room temperature for 10 min. 5 uL of peptide solution containing ULight-4E-BP1 peptide (final concentrations 50 nM) in the assay buffer was subsequently added into each well to initiate the reaction. By incubation at room temperature for 1 hour, 10 uL of 2 nM Eu-anti-phospho-4E BP1 antibody was then added for detection. After one hour incubation at room temperature, all samples were subjected to read the TR-FRET signal on Envision with excitation at 340 nm and emission fluorescence at 615 nm and 665 nm. IC50 values were then calculated by plotting dose-response curves and then using the XLfit application in Excel software.

TABLE 8
Biochemical Data (TR-FRET)

| | CDK1 | CDK2 | CDK2 | |
|---|---|---|---|---|
| | cyclinB | cyclinA2 | cyclinE1 | GSK3β |
| Example | activity assay: | activity assay: | activity assay: | activity assay: |
| Number | IC50 (nM) | IC50 (nM) | IC50 (nM) | IC50 (nM) |
| 4 | B | A | A | B |
| 5 | D | A | A | C |
| 6 | B | A | A | B |
| 7 | B | A | A | D |
| 8 | C | A | A | C |
| 9 | B | A | A | B |
| 10 | A | A | A | B |
| 11 | A | A | A | D |
| 12 | A | A | A | B |
| 13 | B | A | A | B |
| 14 | B | A | A | D |
| 15 | B | A | A | B |
| 16 | B | A | A | C |
| 17 | A | A | A | B |
| 18 | C | A | A | B |
| 19 | B | A | A | B |
| 20 | C | A | A | D |
| 21 | B | A | A | B |
| 22 | A | A | A | C |
| 23 | B | A | A | B |
| 24 | D | C | B | D |
| 25 | B | A | A | B |
| 26 | B | A | A | B |
| 27 | B | A | A | D |
| 28 | A | A | A | B |
| 29 | B | A | A | B |
| 30 | B | A | A | D |
| 31 | B | A | A | B |
| 32 | B | A | A | C |
| 33 | A | A | A | D |
| 34 | B | A | A | C |
| 35 | D | D | D | D |
| 36 | B | A | A | B |
| 37 | B | A | A | D |
| 38 | A | A | A | D |
| 39 | B | A | A | B |
| 40 | B | A | A | D |
| 41 | B | A | A | C |
| 42 | B | A | A | C |
| 43 | D | B | B | D |
| 44 | D | B | B | D |
| 45 | A | A | A | D |
| 46 | B | A | A | B |
| 47 | B | A | A | D |
| 48 | D | B | B | D |
| 49 | B | A | A | D |
| 50 | B | A | A | B |
| 51 | B | C | B | D |
| 52 | D | B | A | D |
| 53 | C | A | A | D |
| 54 | D | A | A | D |
| 55 | B | A | A | B |
| 56 | B | A | A | B |
| 57 | B | A | A | B |
| 58 | B | A | A | C |
| 59 | B | A | A | C |
| 60 | B | A | A | D |
| 61 | A | A | A | C |
| 62 | B | A | A | D |
| 63 | B | A | A | D |
| 64 | B | A | A | B |
| 65 | B | A | A | B |
| 66 | A | A | A | B |
| 67 | B | A | A | D |
| 68 | A | A | A | B |
| 69 | B | A | A | D |
| 70 | A | A | A | A |
| 71 | B | A | A | B |
| 72 | A | A | A | D |

### Cell Proliferation

Cancer cell lines, namely, OVCAR3, TOV21G were seeded onto 96 well cell culture plates at a density of 2500, 1000 or 3000 cells/well, respectively and cultured overnight. Cells treated with test compounds at 3-fold dose titration for 7 days at maximum final DMSO concentration of 0.01%. The CyQUANT® Cell Proliferation Assay, a highly sensitive fluorescence-based method for quantifying cells and assessing cell proliferation and cytotoxicity was employed, following manufacturer's procedure (ThermnoFisher, Cataloge #C35012). Plates were scanned with a laser-based Acumen Cellistra instrument by TTP LabTech at Ex/Em 480/520 nm. % Inhibition is calculated based on DMSO control wells on the same plate (ie, DMSO wells have 100% growth and 0% growth inhibition).

TABLE 9
Cell proliferation data

| | Anti-proliferation assay: | Anti-proliferation |
|---|---|---|
| Example | IC50 (nM) [Cell Line: | assay: IC50 (nM) |
| Number | OVCAR3] | [Cell Line: TOV21G] |
| 1 | CC | CC |
| 2 | AA | CC |
| 3 | AA | CC |
| 4 | BB | CC |
| 5 | CC | CC |
| 6 | AA | CC |
| 7 | AA | CC |
| 8 | CC | CC |
| 9 | AA | CC |
| 10 | AA | CC |
| 11 | AA | CC |
| 12 | AA | CC |
| 15 | AA | CC |
| 16 | AA | CC |
| 17 | AA | AA |
| 19 | AA | AA |
| 21 | BB | CC |
| 22 | AA | AA |
| 23 | AA | CC |
| 25 | AA | CC |
| 26 | AA | CC |
| 27 | CC | CC |
| 28 | AA | AA |
| 29 | BB | CC |
| 30 | BB | CC |
| 31 | AA | AA |
| 32 | AA | CC |
| 33 | AA | AA |
| 34 | BB | CC |
| 36 | AA | AA |
| 37 | CC | CC |
| 38 | AA | CC |
| 40 | AA | CC |
| 41 | AA | AA |
| 42 | AA | AA |
| 45 | CC | CC |
| 46 | AA | BB |
| 47 | AA | AA |
| 49 | AA | BB |
| 50 | AA | AA |
| 55 | AA | CC |
| 56 | AA | CC |
| 57 | AA | BB |
| 58 | AA | CC |
| 59 | AA | BB |
| 60 | AA | CC |
| 61 | AA | AA |
| 62 | AA | CC |
| 63 | AA | CC |
| 64 | AA | CC |
| 65 | AA | BB |
| 66 | AA | CC |
| 67 | AA | CC |
| 68 | AA | AA |
| 69 | BB | CC |
| 70 | AA | AA |

In Table 7 and Table 8, a compound described herein may have an IC50 of CDK1 cyclinB, CDK2 cyclinA2, CDK2 cyclinEI or GSK3β 3. “A” refers to an IC50 less than 50 nM; “B” refers to an IC50 from 50 nM to 0.5 μM; “C” refers to an IC50 from 0.5 μM to 1 uM; and “D” refers to an IC50 greater than 1 μM. In Table 9, a compound was tested of anti-proliferation assay in both OVCAR3 and TOV21G cell lines. “AA” refers to an IC50 less than or equal to 0.5 μM; “BB” refers to an IC50 from 0.5 Mμ to 1 uM, and “CC” refers to an IC50 greater than or equal to 1 μM.

Although the present invention has been described in detail with preferred embodiments, those of ordinary skill in the art should understand that modifications, variations, and equivalent replacements made to the present invention within the scope of the present invention belong to the protection of the present invention.

Applicant's disclosure is described herein in preferred embodiments with reference to the FIGURES, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1-89. (canceled)

90. A compound having the structural Formula I: 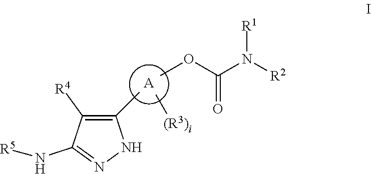
or a pharmaceutically acceptable salt thereof,
wherein
Ring A is a 4- to 7-membered carbocyclic ring;
each of R¹ and R² is independently selected from the group consisting of H, unsubstituted C₁-C₆ alkyl, and C₃-C₆ unsubstituted carbocyclic ring; or R¹ and R², together with the N atom they are bound to, are joined to form a 4- to 7-membered, unsubstituted heterocyclic ring;
each R³ is independently selected from the group consisting of halogen, OH, CN, unsubstituted C₁₋₆ alkyl, unsubstituted C₁₋₆ alkoxy, NO₂, NRR′, or two R³'s, along with the carbon atom(s) they are bound to, form a 3- to 5-membered unsubstituted carbocyclic ring;
R⁴ is H, halogen, C₁₋₄ alkyl, CN, NRR′, or C(O)NRR′;
R⁵ is 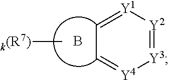
wherein
Ring B is a 4- to 7-membered carbocyclic or heterocyclic ring optionally comprising a —S(O)₂—, —C(O)—, —S(O)₂NR—, —S(O)₂NRC(O)—, —C(O)NR—, —OC(O)NR—, or —C(O)NRC(O)— group;
each of Y¹ and Y² is CR⁶ or N, and each of Y³ and Y⁴ is C, CR⁶ or N; provided that not more than one of Y¹, Y², Y³ and Y⁴ is N, and either Y³ or Y⁴ is C and being the positions where R⁵ may be bonded to the rest of the compound;
each R⁶ is independently selected from the group consisting of halogen, OH, CN, unsubstituted C₁₋₆ alkyl, unsubstituted C₁₋₄ alkoxy, and C(O)NRR′;
each R⁷ is independently selected from the group consisting of halogen, OR, CN, unsubstituted C₁₋₆ alkyl, and unsubstituted C₁₋₄ alkoxy, or two R⁷'s, along with the carbon atom(s) they are bound to, form a 3- to 6-membered unsubstituted carbocyclic or heterocyclic ring;
each of R and R′ is independently selected from H, unsubstituted C₁₋₆ alkyl, or unsubstituted 4- to 6-membered carbocyclic ring, or where R and R′ are attached to the same N atom, together form an unsubstituted 4- to 6-membered heterocyclic ring;
i is 0, 1,2, 3,4 or 5; and
k is 0, 1, 2, 3, 4 or 5.

91. The compound or a pharmaceutically acceptable salt thereof of claim 90, wherein Ring B is selected from: 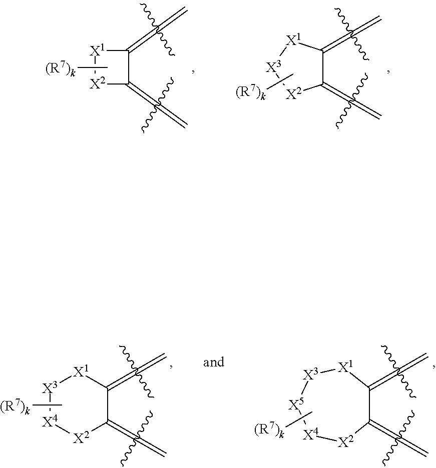
wherein
each of X¹, X², X³, X⁴ and X⁵ is independently selected from S(O)₂, C(O), NH, CH2 and O, provided that two neighboring X¹, X², X³, X⁴ or X₅ are not each selected from S(O)₂ and C(O); and two neighboring X¹, X², X³, X⁴ or X₅ are not each selected from NH and O.

92. The compound or a pharmaceutically acceptable salt thereof of claim 90, wherein R⁵ is selected from: 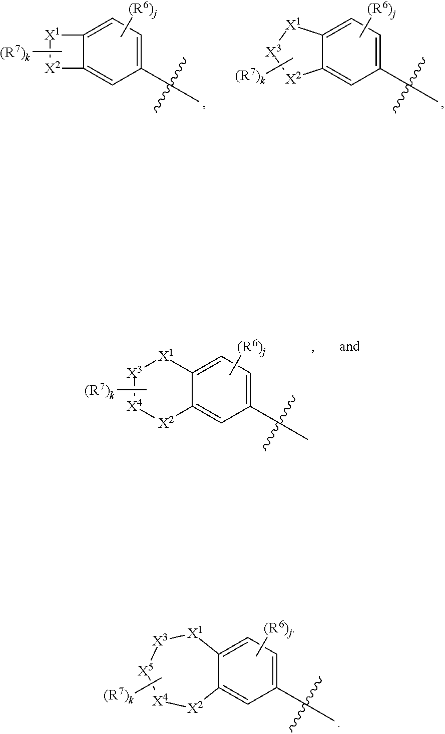
wherein
each of X¹, X², X³, X⁴ and X⁵ is independently selected from S(O)₂, C(O), NH, CH2 and O, provided that two neighboring X¹, X², X³, X⁴ or X₅ are not each selected from S(O)₂ and C(O); and two neighboring X¹, X², X³, X⁴ or X₅ are not each selected from NH and O.

93. The compound or a pharmaceutically acceptable salt thereof of claim 90, wherein R⁵ is selected from: 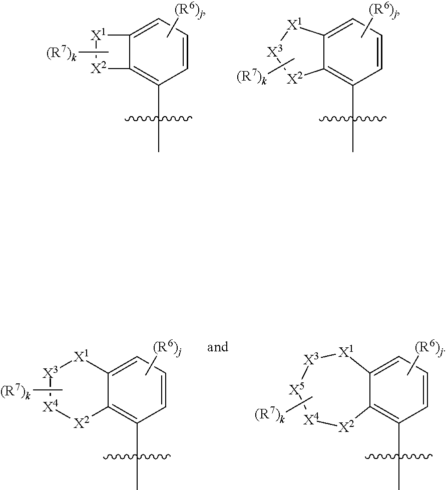
wherein
each of X¹, X², X³, X⁴ and X⁵ is independently selected from S(O)₂, C(O), NH, CH2 and O, provided that two neighboring X¹, X², X³, X⁴ or X₅ are not each selected from S(O)₂ and C(O); and two neighboring X¹, X², X³, X⁴ or X₅ are not each selected from NH and O.

94. The compound or a pharmaceutically acceptable salt thereof of claim 90, wherein R⁵ is selected from: 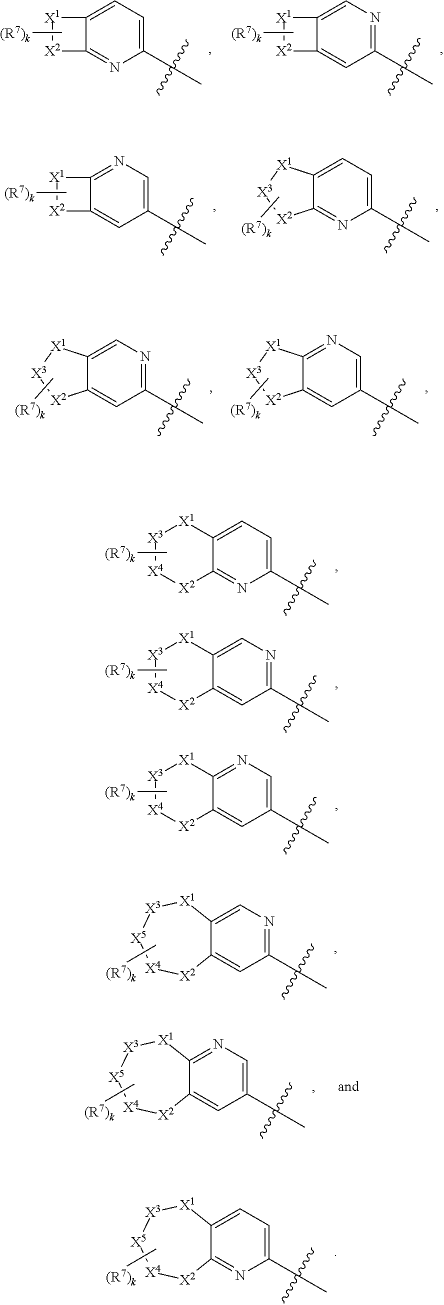
wherein
each of X¹, X², X³, X⁴ and X⁵ is independently selected from S(O)₂, C(O), NH, CH2 and O, provided that two neighboring X¹, X², X³, X⁴ or are not each selected from S(O)₂ and C(O); and two neighboring X¹, X², X³, X⁴ or X are not each selected from NH and O.

95. The compound or a pharmaceutically acceptable salt thereof of claim 90, wherein R⁵ is selected from: 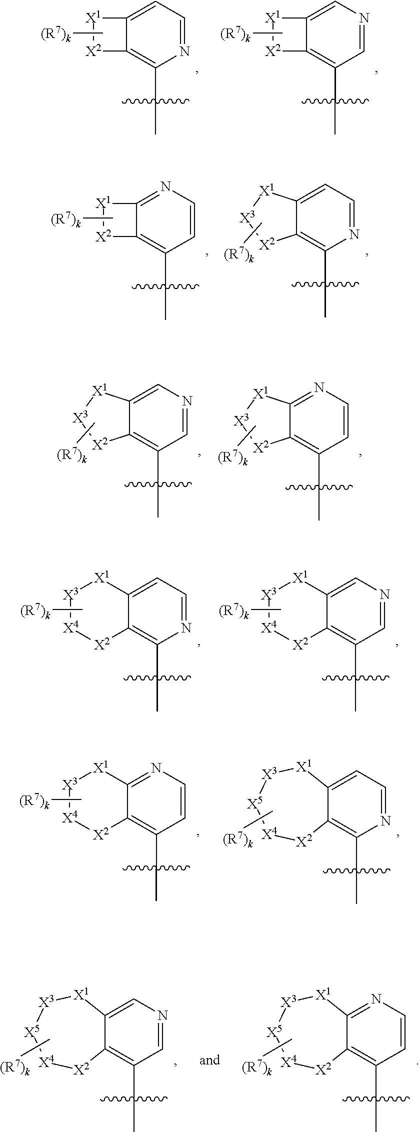
wherein
each of X¹, X², X³, X⁴ and X⁵ is independently selected from S(O)₂, C(O), NH, CH2 and O, provided that two neighboring X¹, X², X⁴ or are not each selected from S(O)₂ and C(O); and two neighboring X¹, X², X³, X⁴ or are not each selected from NH and O.

96. The compound or a pharmaceutically acceptable salt thereof of claim 90, wherein R⁵ is selected from:

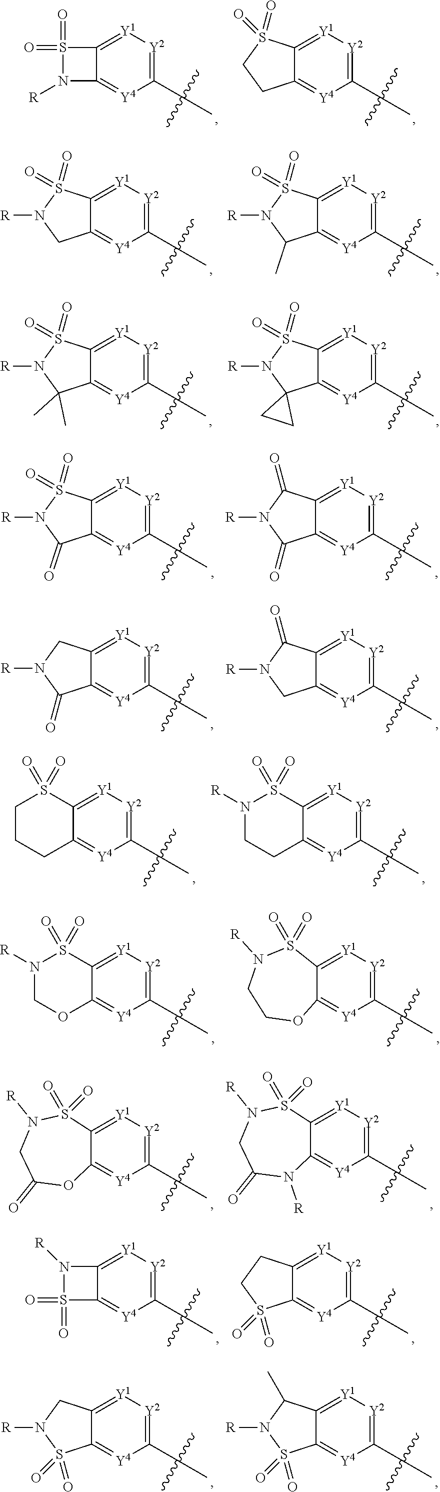

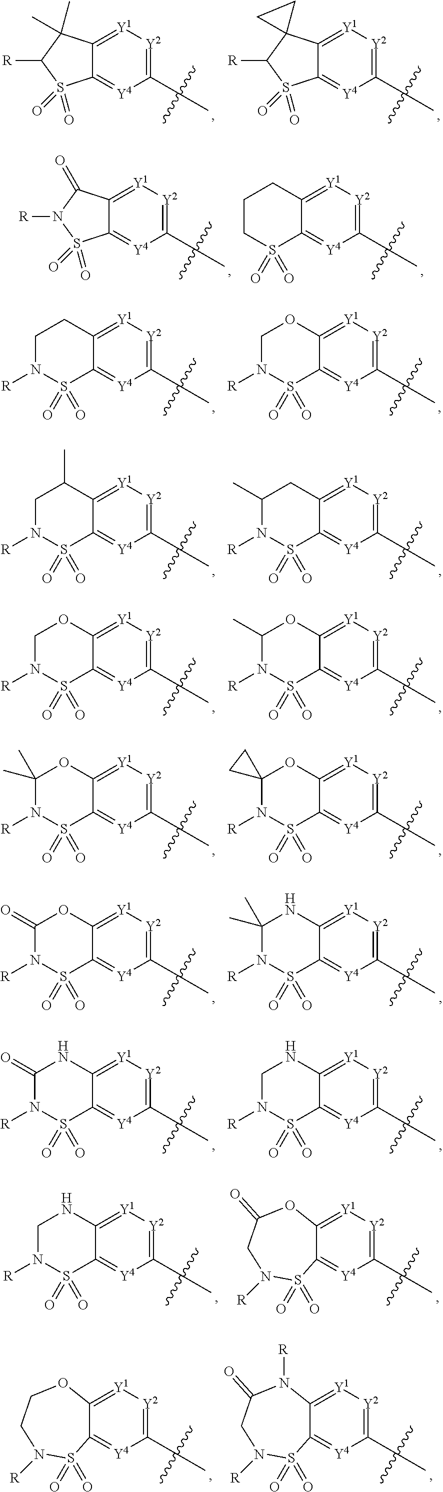

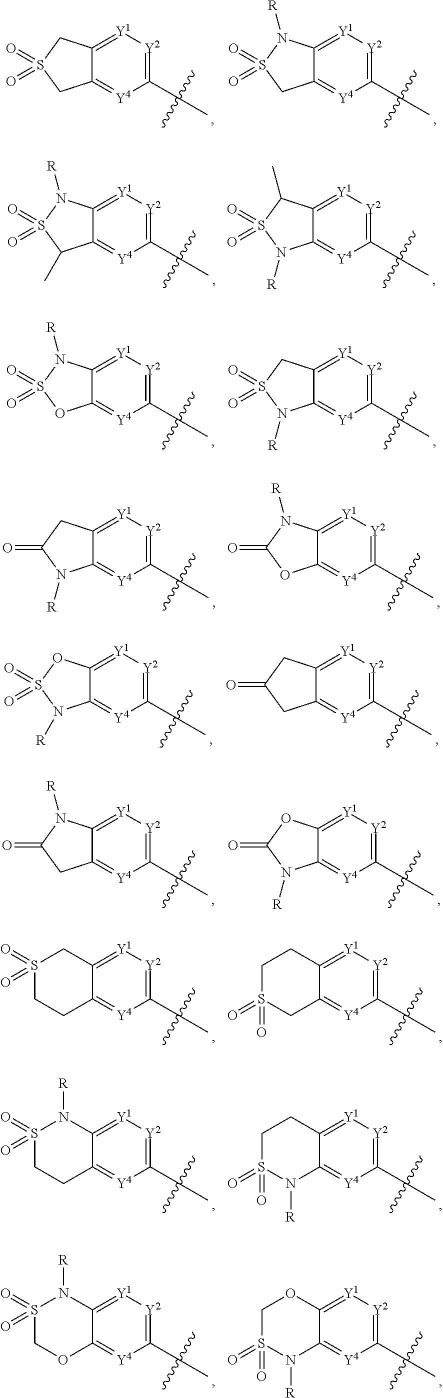

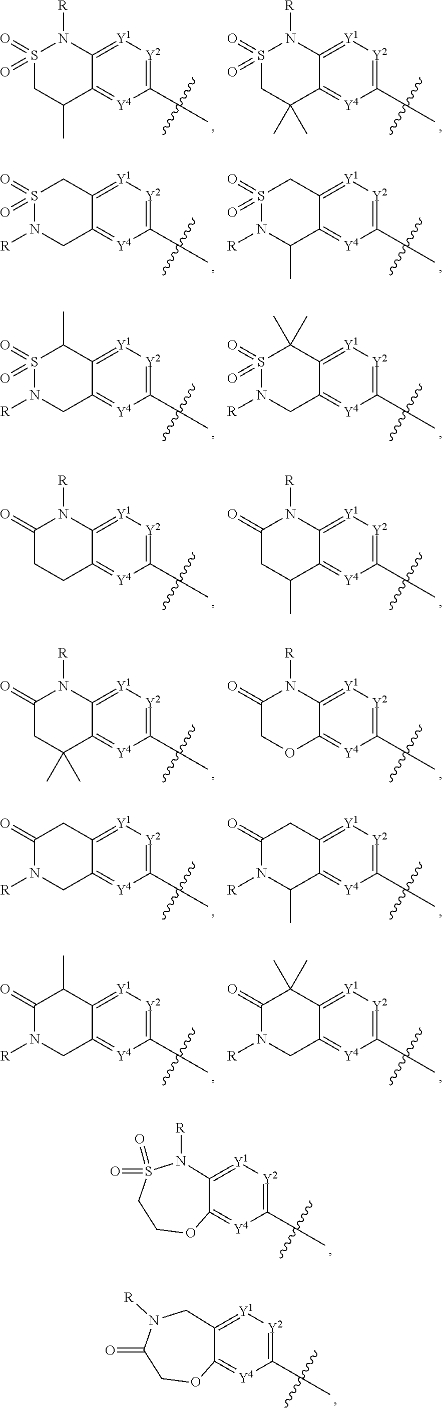

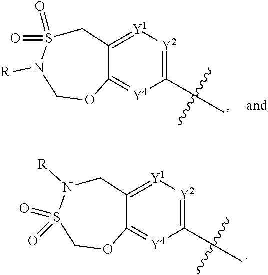

97. The compound or a pharmaceutically acceptable salt thereof of claim 96, wherein (1) Y¹ is N, Y² is CH and Y⁴ is CH, (2) Y¹ is CH, Y² is N and Y⁴ is CH, (3)Y¹ is CH, Y² is CH and Y⁴ is N, or (4) each of Y¹, Y² and Y⁴ is CH.

98. The compound or a pharmaceutically acceptable salt thereof of claim 90, wherein R⁵ is selected from:

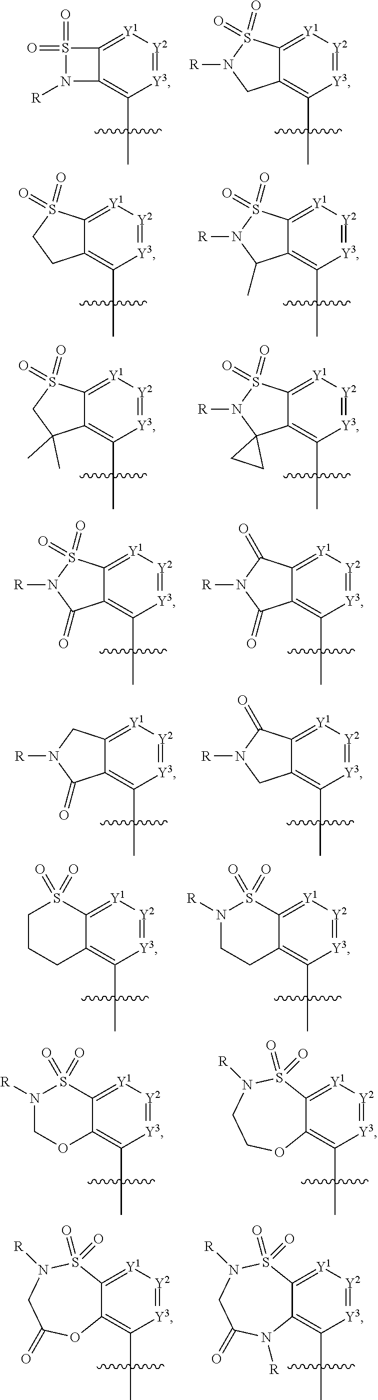

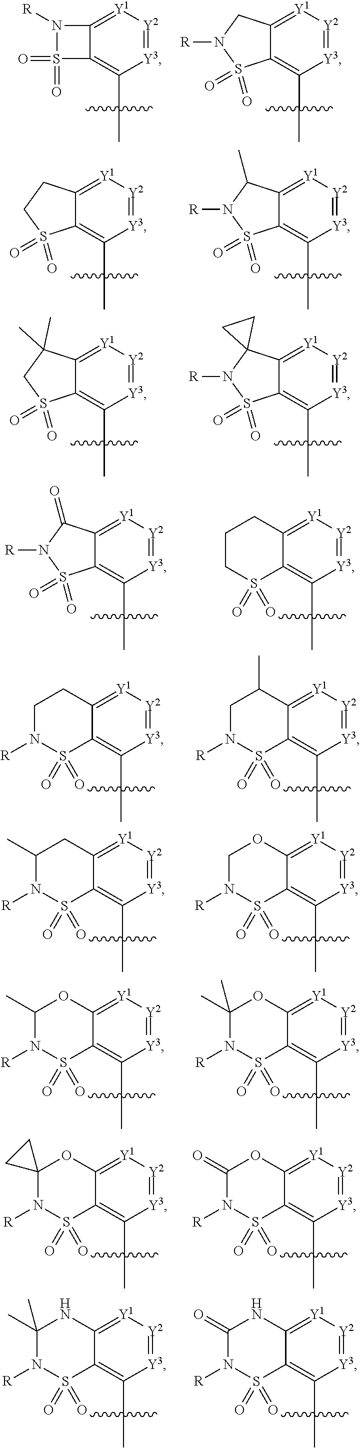

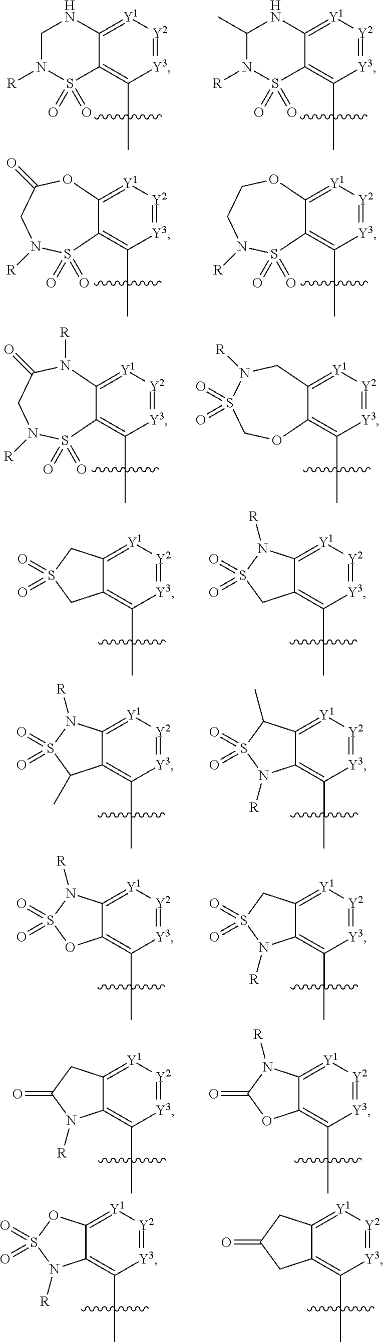

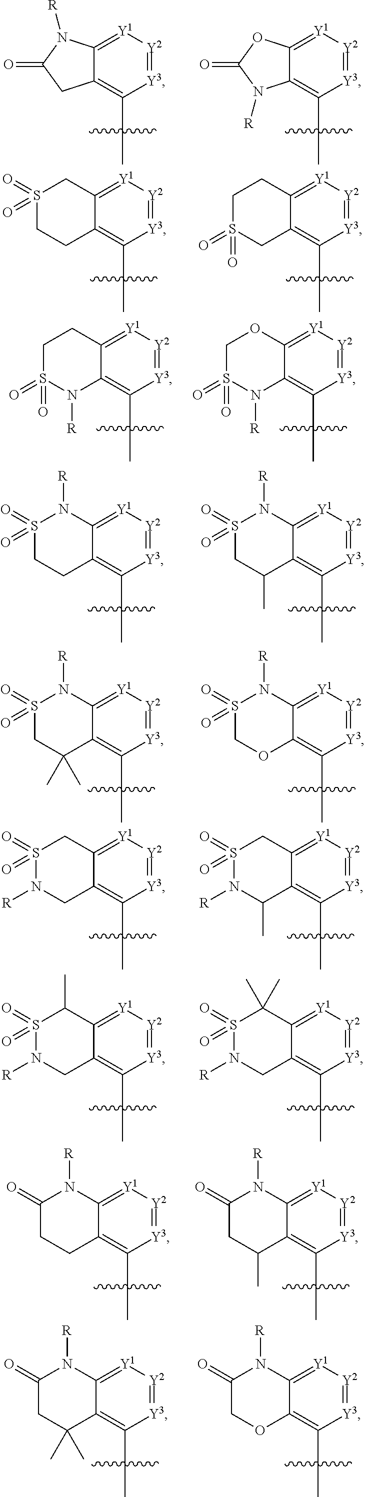

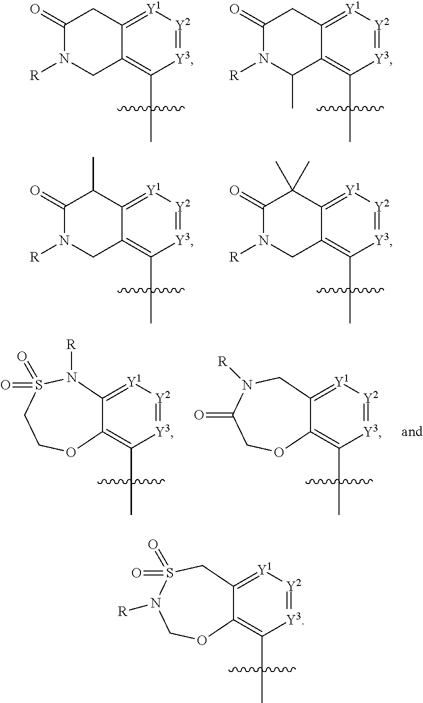

99. The compound or a pharmaceutically acceptable salt thereof of claim 98, wherein (1) Y¹ is N, Y² is CH and Y³ is CH, (2) Y¹ is CH, Y² is N and Y³ is CH, (3) Y¹ is CH, Y² is CH and Y³ is N, or (4) each of Y¹, Y² and Y³ is CH.

100. The compound or a pharmaceutically acceptable salt thereof of claim 90, wherein R¹ is H and R² is an unsubstituted linear or branched C₁-C₆ alkyl.

101. The compound or a pharmaceutically acceptable salt thereof of claim 90, wherein Ring A is selected from:

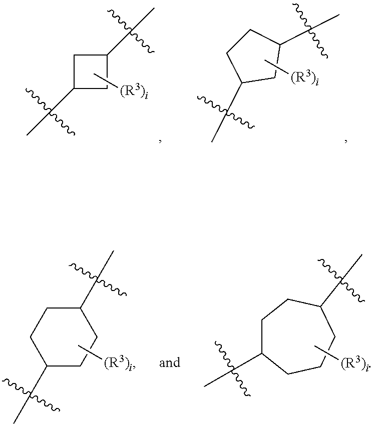

102. The compound or a pharmaceutically acceptable salt thereof of claim 90, wherein Ring A is selected from:

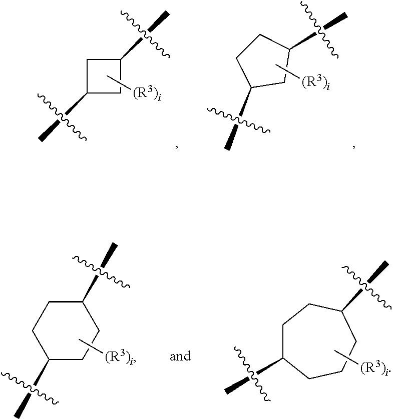

103. The compound or a pharmaceutically acceptable salt thereof of claim 90, wherein i is 0.

104. A compound selected from: 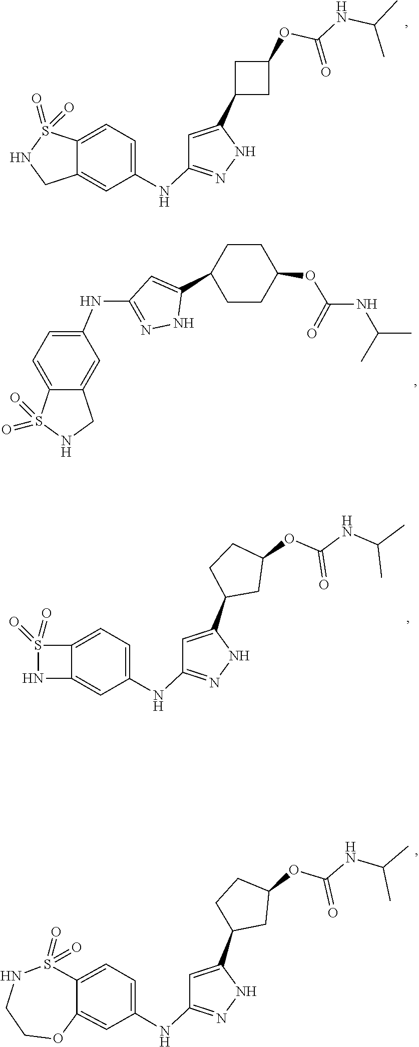 [SMILES: CC(C)NC(=O)O[C@H]1C[C@@H](c2cc(Nc3ccc4c(c3)CNS4(=O)=O)n[nH]2)C1 ; CC(C)NC(=O)O[C@H]1CC[C@@H](c2cc(Nc3ccc4c(c3)CNS4(=O)=O)n[nH]2)CC1 ; CC(C)NC(=O)O[C@@H]1CC[C@H](c2cc(Nc3ccc4c(c3)NS4(=O)=O)n[nH]2)C1 ; CC(C)NC(=O)O[C@@H]1CC[C@H](c2cc(Nc3ccc4c(c3)OCCNS4(=O)=O)n[nH]2)C1] 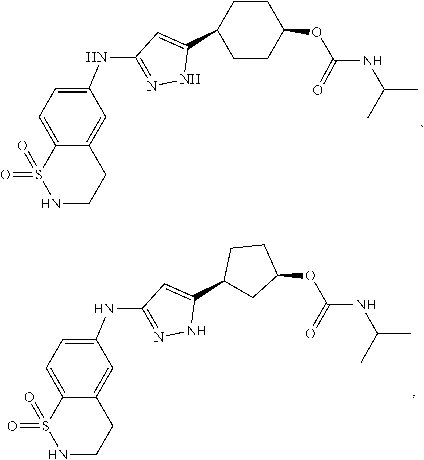 [SMILES: CC(C)NC(=O)O[C@H]1CC[C@@H](c2cc(Nc3ccc4c(c3)CCNS4(=O)=O)n[nH]2)CC1 ; CC(C)NC(=O)O[C@@H]1CC[C@H](c2cc(Nc3ccc4c(c3)CCNS4(=O)=O)n[nH]2)C1] 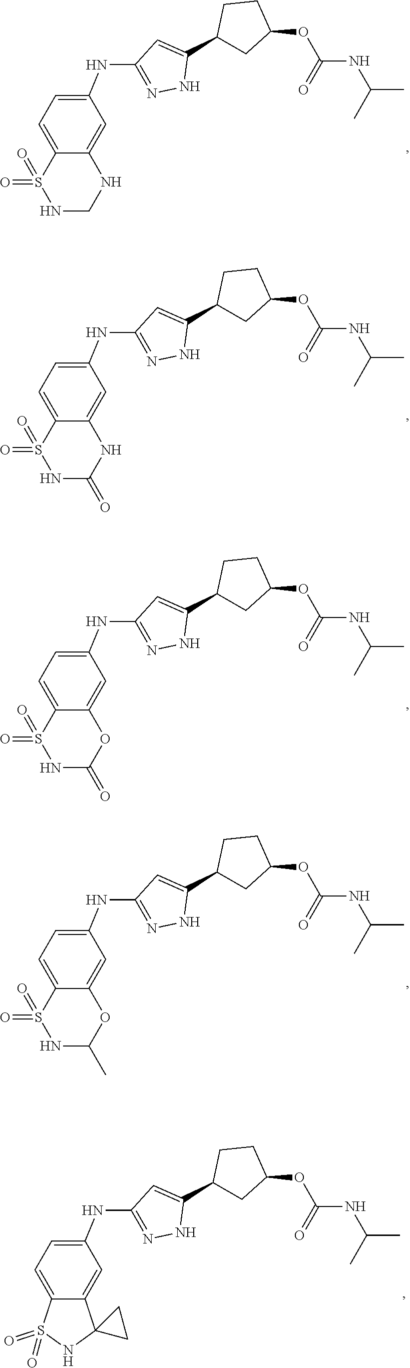 [SMILES: CC(C)NC(=O)O[C@@H]1CC[C@H](c2cc(Nc3ccc4c(c3)NCNS4(=O)=O)n[nH]2)C1 ; CC(C)NC(=O)O[C@@H]1CC[C@H](c2cc(Nc3ccc4c(c3)NC(=O)NS4(=O)=O)n[nH]2)C1 ; CC(C)NC(=O)O[C@@H]1CC[C@H](c2cc(Nc3ccc4c(c3)OC(=O)NS4(=O)=O)n[nH]2)C1 ; CC(C)NC(=O)O[C@@H]1CC[C@H](c2cc(Nc3ccc4c(c3)OC(C)NS4(=O)=O)n[nH]2)C1 ; CC(C)NC(=O)O[C@@H]1CC[C@H](c2cc(Nc3ccc4c(c3)C3(CC3)NS4(=O)=O)n[nH]2)C1] 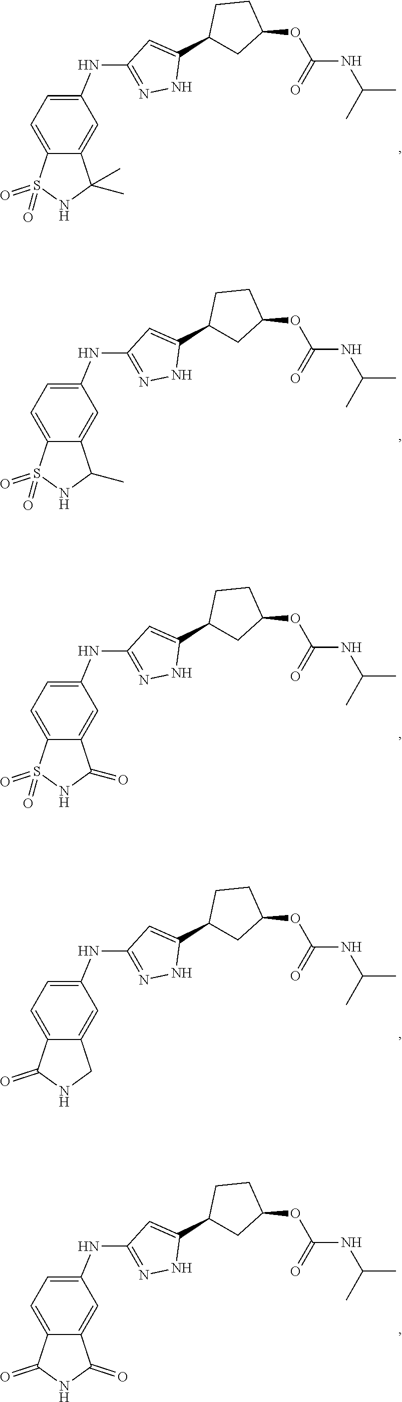 [SMILES: CC(C)NC(=O)O[C@@H]1CC[C@H](c2cc(Nc3ccc4c(c3)C(C)(C)NS4(=O)=O)n[nH]2)C1 ; CC(C)NC(=O)O[C@@H]1CC[C@H](c2cc(Nc3ccc4c(c3)C(C)NS4(=O)=O)n[nH]2)C1 ; CC(C)NC(=O)O[C@@H]1CC[C@H](c2cc(Nc3ccc4c(c3)C(=O)NS4(=O)=O)n[nH]2)C1 ; CC(C)NC(=O)O[C@@H]1CC[C@H](c2cc(Nc3ccc4c(c3)CNC4=O)n[nH]2)C1 ; CC(C)NC(=O)O[C@@H]1CC[C@H](c2cc(Nc3ccc4c(c3)C(=O)NC4=O)n[nH]2)C1] 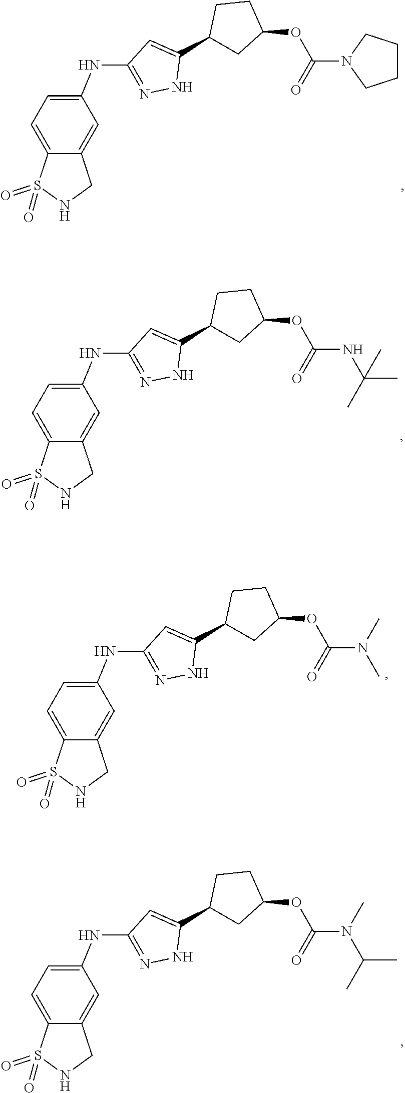 [SMILES: O=C(O[C@@H]1CC[C@H](c2cc(Nc3ccc4c(c3)CNS4(=O)=O)n[nH]2)C1)N1CCCC1 ; CC(C)(C)NC(=O)O[C@@H]1CC[C@H](c2cc(Nc3ccc4c(c3)CNS4(=O)=O)n[nH]2)C1 ; CN(C)C(=O)O[C@@H]1CC[C@H](c2cc(Nc3ccc4c(c3)CNS4(=O)=O)n[nH]2)C1 ; CC(C)N(C)C(=O)O[C@@H]1CC[C@H](c2cc(Nc3ccc4c(c3)CNS4(=O)=O)n[nH]2)C1] 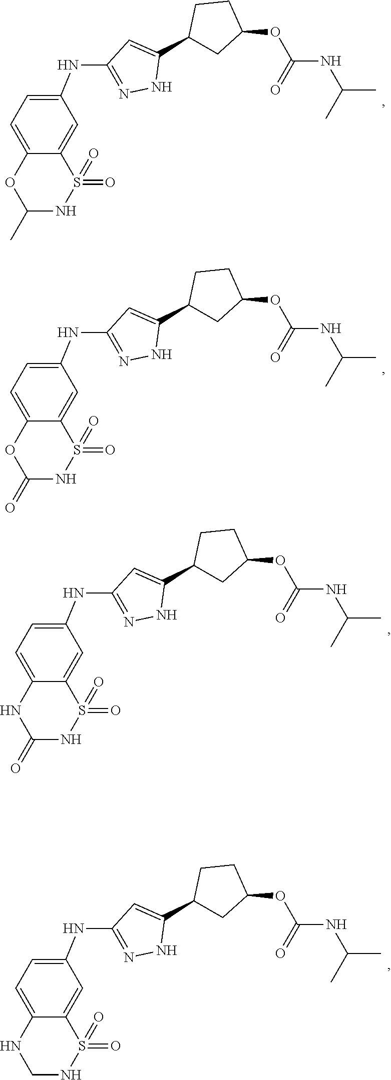 [SMILES: CC(C)NC(=O)O[C@@H]1CC[C@H](c2cc(Nc3ccc4c(c3)S(=O)(=O)NC(C)O4)n[nH]2)C1 ; CC(C)NC(=O)O[C@@H]1CC[C@H](c2cc(Nc3ccc4c(c3)S(=O)(=O)NC(=O)O4)n[nH]2)C1 ; CC(C)NC(=O)O[C@@H]1CC[C@H](c2cc(Nc3ccc4c(c3)S(=O)(=O)NC(=O)N4)n[nH]2)C1 ; CC(C)NC(=O)O[C@@H]1CC[C@H](c2cc(Nc3ccc4c(c3)S(=O)(=O)NCN4)n[nH]2)C1] 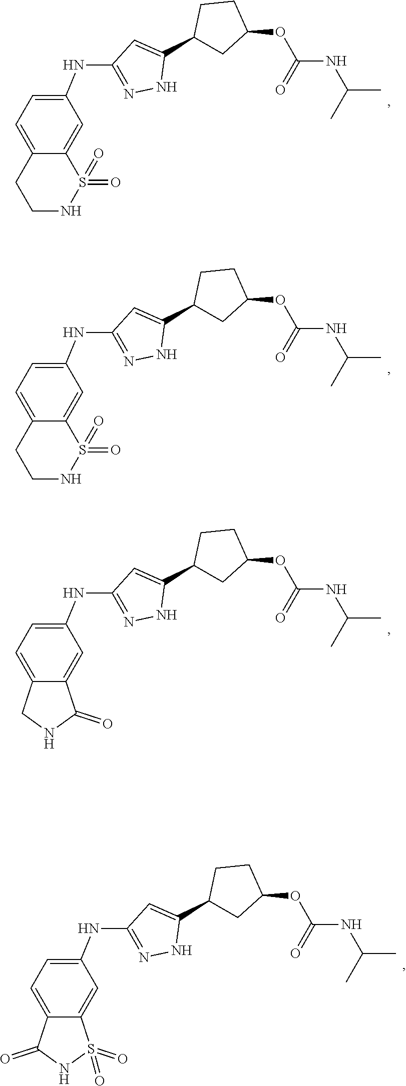 [SMILES: CC(C)NC(=O)O[C@@H]1CC[C@H](c2cc(Nc3ccc4c(c3)S(=O)(=O)NCC4)n[nH]2)C1 ; CC(C)NC(=O)O[C@@H]1CC[C@H](c2cc(Nc3ccc4c(c3)S(=O)(=O)NCC4)n[nH]2)C1 ; CC(C)NC(=O)O[C@@H]1CC[C@H](c2cc(Nc3ccc4c(c3)C(=O)NC4)n[nH]2)C1 ; CC(C)NC(=O)O[C@@H]1CC[C@H](c2cc(Nc3ccc4c(c3)S(=O)(=O)NC4=O)n[nH]2)C1] 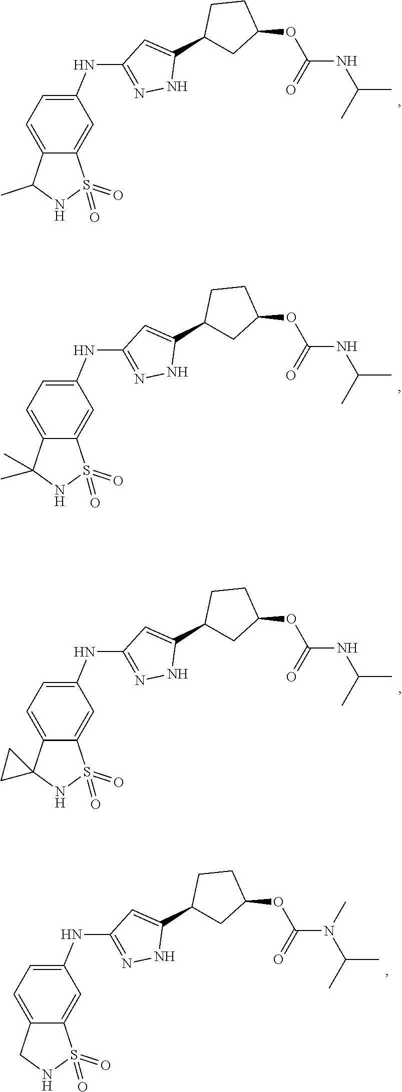 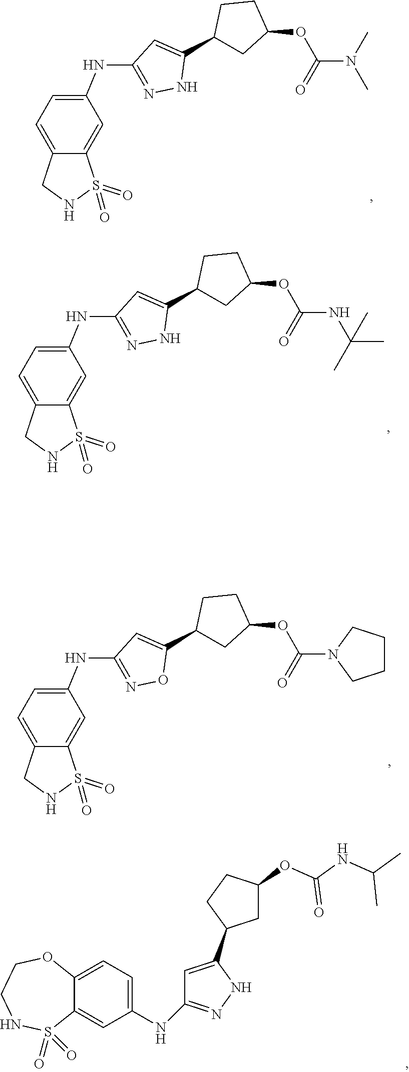 [SMILES: CN(C)C(=O)O[C@@H]1CC[C@H](c2cc(Nc3ccc4c(c3)S(=O)(=O)NC4)n[nH]2)C1 ; CC(C)(C)NC(=O)O[C@@H]1CC[C@H](c2cc(Nc3ccc4c(c3)S(=O)(=O)NC4)n[nH]2)C1 ; O=C(O[C@@H]1CC[C@H](c2cc(Nc3ccc4c(c3)S(=O)(=O)NC4)no2)C1)N1CCCC1 ; CC(C)NC(=O)O[C@@H]1CC[C@H](c2cc(Nc3ccc4c(c3)S(=O)(=O)NCCO4)n[nH]2)C1] 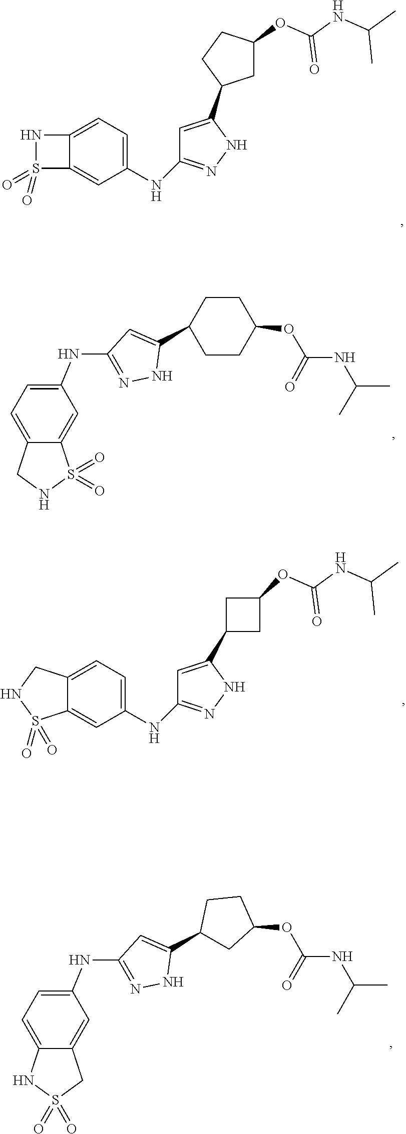 [SMILES: CC(C)NC(=O)O[C@@H]1CC[C@H](c2cc(Nc3ccc4c(c3)S(=O)(=O)N4)n[nH]2)C1 ; CC(C)NC(=O)O[C@H]1CC[C@@H](c2cc(Nc3ccc4c(c3)S(=O)(=O)NC4)n[nH]2)CC1 ; CC(C)NC(=O)O[C@H]1C[C@@H](c2cc(Nc3ccc4c(c3)S(=O)(=O)NC4)n[nH]2)C1 ; CC(C)NC(=O)O[C@@H]1CC[C@H](c2cc(Nc3ccc4c(c3)CS(=O)(=O)N4)n[nH]2)C1] 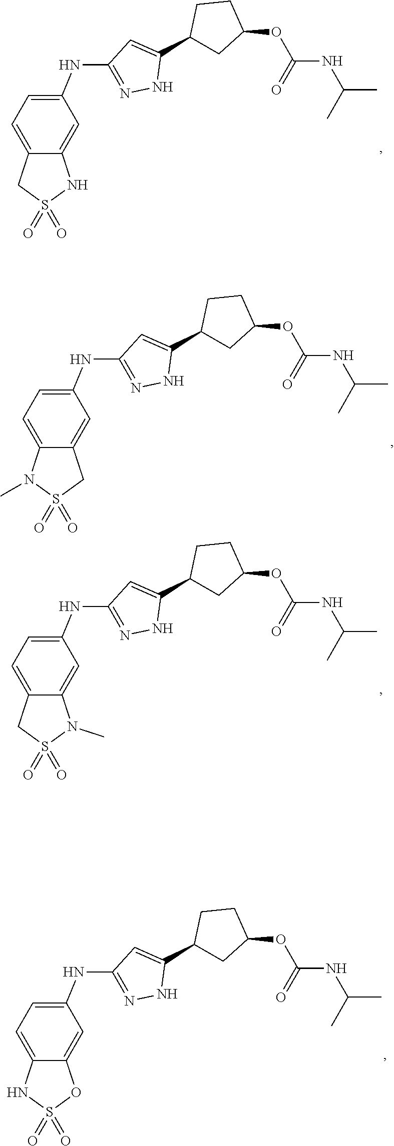 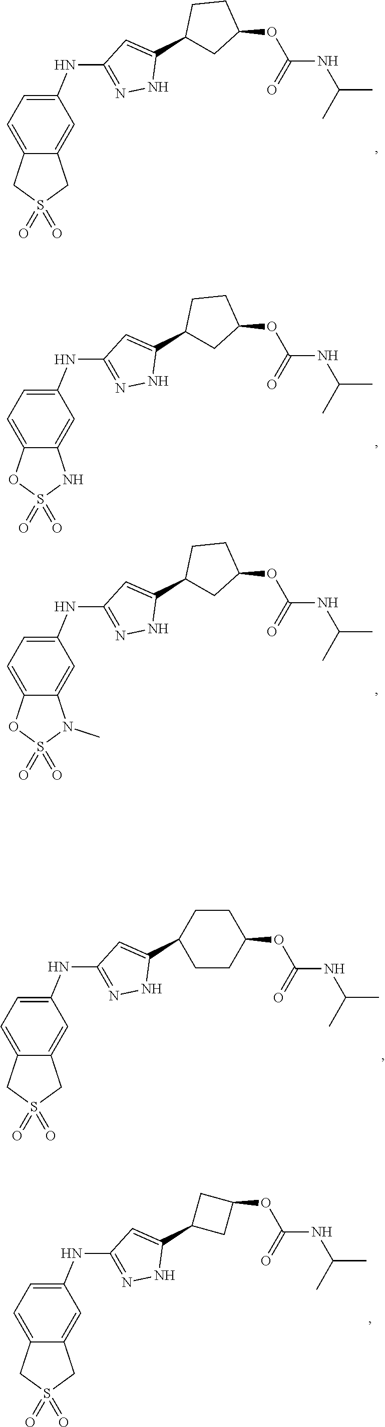 [SMILES: CC(C)NC(=O)O[C@@H]1CC[C@H](c2cc(Nc3ccc4c(c3)CS(=O)(=O)C4)n[nH]2)C1 ; CC(C)NC(=O)O[C@@H]1CC[C@H](c2cc(Nc3ccc4c(c3)NS(=O)(=O)O4)n[nH]2)C1 ; CC(C)NC(=O)O[C@@H]1CC[C@H](c2cc(Nc3ccc4c(c3)N(C)S(=O)(=O)O4)n[nH]2)C1 ; CC(C)NC(=O)O[C@H]1CC[C@@H](c2cc(Nc3ccc4c(c3)CS(=O)(=O)C4)n[nH]2)CC1 ; CC(C)NC(=O)O[C@H]1C[C@@H](c2cc(Nc3ccc4c(c3)CS(=O)(=O)C4)n[nH]2)C1] 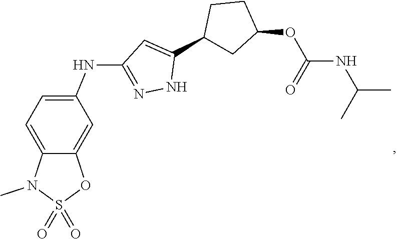 [SMILES: CC(C)NC(=O)O[C@@H]1CC[C@H](c2cc(Nc3ccc4c(c3)OS(=O)(=O)N4C)n[nH]2)C1] 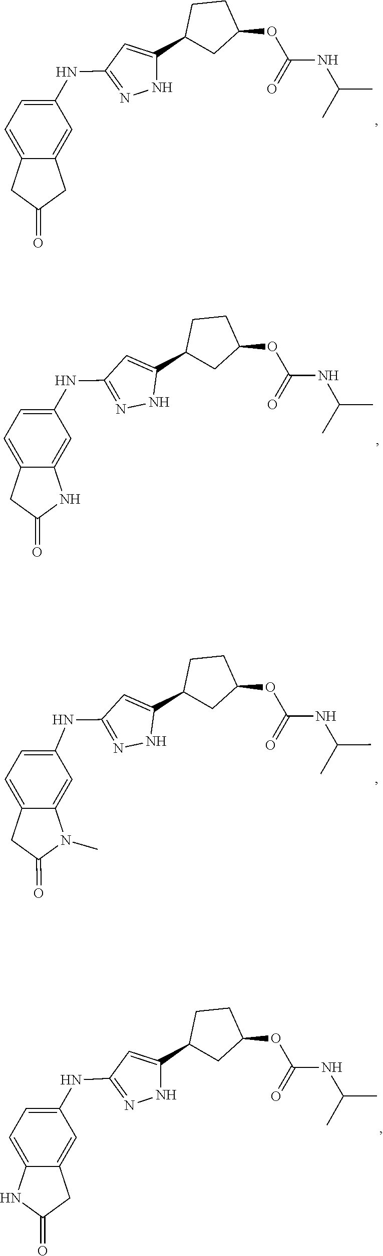 [SMILES: CC(C)NC(=O)O[C@@H]1CC[C@H](c2cc(Nc3ccc4c(c3)CC(=O)C4)n[nH]2)C1 ; CC(C)NC(=O)O[C@@H]1CC[C@H](c2cc(Nc3ccc4c(c3)NC(=O)C4)n[nH]2)C1 ; CC(C)NC(=O)O[C@@H]1CC[C@H](c2cc(Nc3ccc4c(c3)N(C)C(=O)C4)n[nH]2)C1 ; CC(C)NC(=O)O[C@@H]1CC[C@H](c2cc(Nc3ccc4c(c3)CC(=O)N4)n[nH]2)C1] 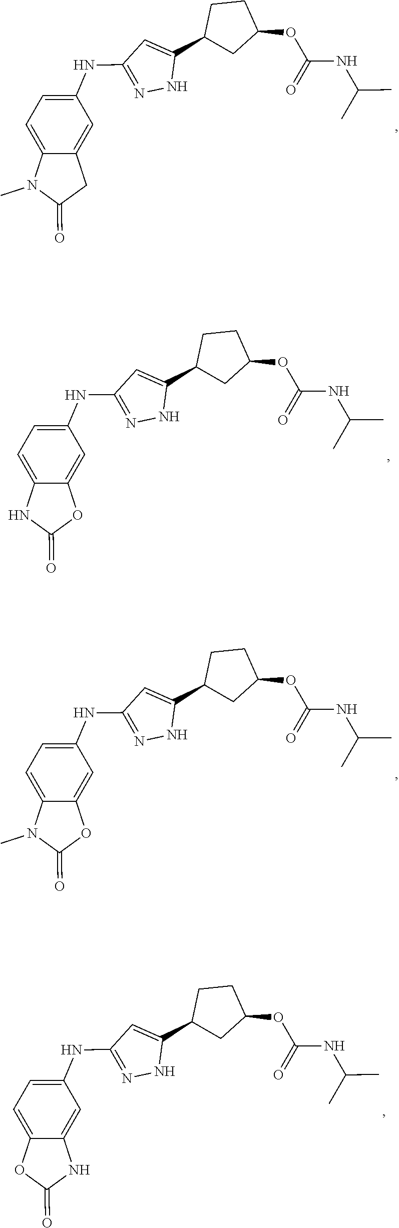 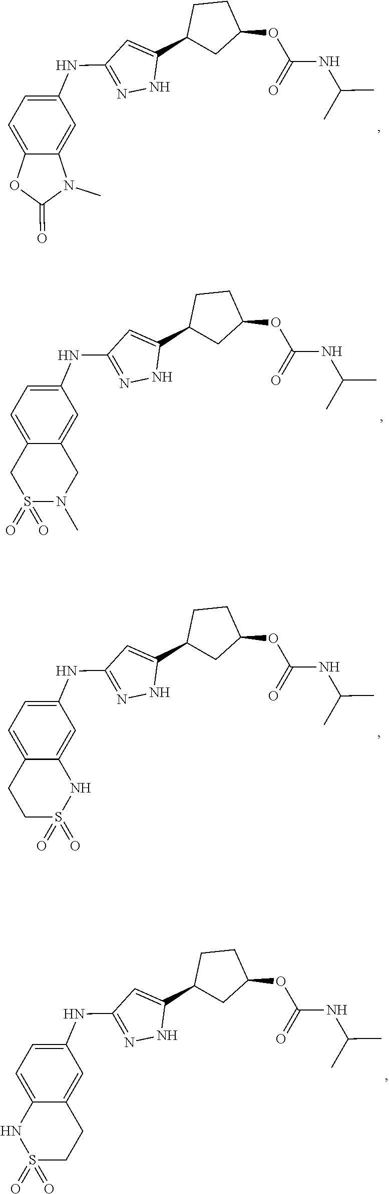 [SMILES: CC(C)NC(=O)O[C@@H]1CC[C@H](c2cc(Nc3ccc4oc(=O)n(C)c4c3)n[nH]2)C1 ; CC(C)NC(=O)O[C@@H]1CC[C@H](c2cc(Nc3ccc4c(c3)CN(C)S(=O)(=O)C4)n[nH]2)C1 ; CC(C)NC(=O)O[C@@H]1CC[C@H](c2cc(Nc3ccc4c(c3)NS(=O)(=O)CC4)n[nH]2)C1 ; CC(C)NC(=O)O[C@@H]1CC[C@H](c2cc(Nc3ccc4c(c3)CCS(=O)(=O)N4)n[nH]2)C1] 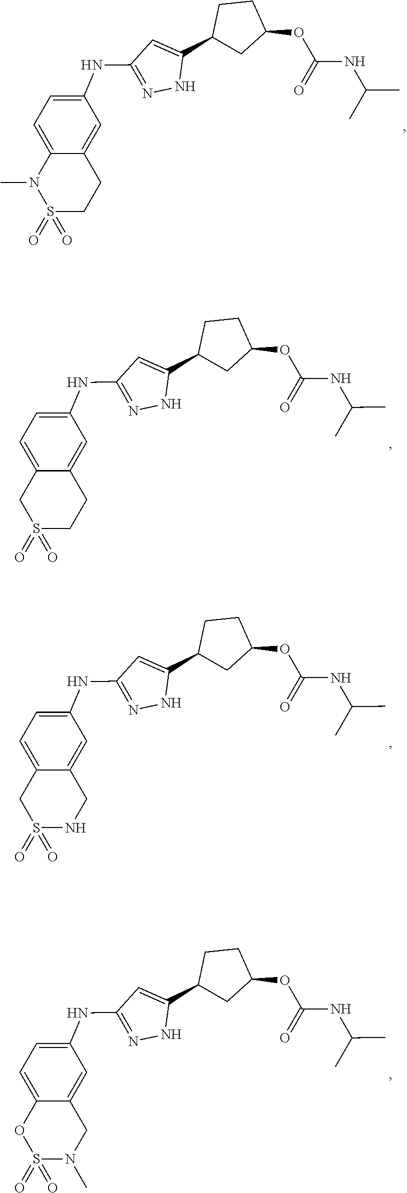 [SMILES: CC(C)NC(=O)O[C@@H]1CC[C@H](c2cc(Nc3ccc4c(c3)CCS(=O)(=O)N4C)n[nH]2)C1 ; CC(C)NC(=O)O[C@@H]1CC[C@H](c2cc(Nc3ccc4c(c3)CCS(=O)(=O)C4)n[nH]2)C1 ; CC(C)NC(=O)O[C@@H]1CC[C@H](c2cc(Nc3ccc4c(c3)CNS(=O)(=O)C4)n[nH]2)C1 ; CC(C)NC(=O)O[C@@H]1CC[C@H](c2cc(Nc3ccc4c(c3)CN(C)S(=O)(=O)O4)n[nH]2)C1] 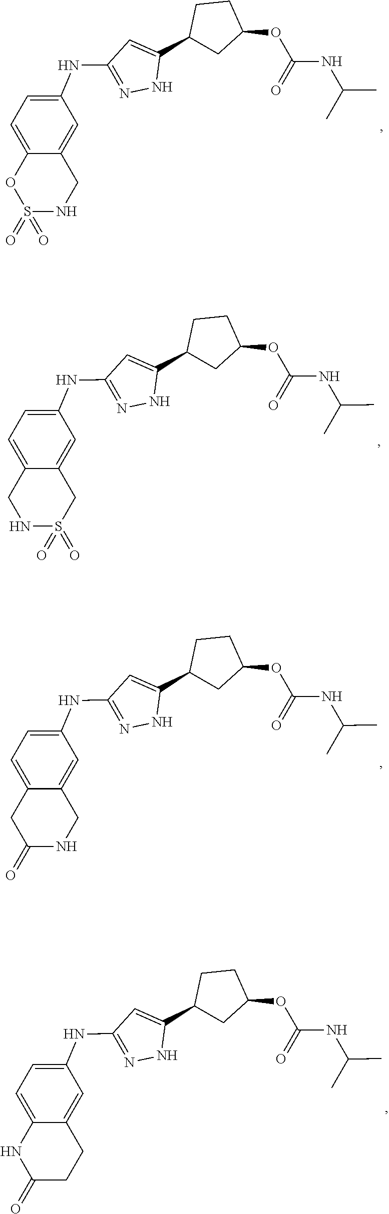 [SMILES: CC(C)NC(=O)O[C@@H]1CC[C@H](c2cc(Nc3ccc4c(c3)CNS(=O)(=O)O4)n[nH]2)C1 ; CC(C)NC(=O)O[C@@H]1CC[C@H](c2cc(Nc3ccc4c(c3)CS(=O)(=O)NC4)n[nH]2)C1 ; CC(C)NC(=O)O[C@@H]1CC[C@H](c2cc(Nc3ccc4c(c3)CNC(=O)C4)n[nH]2)C1 ; CC(C)NC(=O)O[C@@H]1CC[C@H](c2cc(Nc3ccc4c(c3)CCC(=O)N4)n[nH]2)C1] 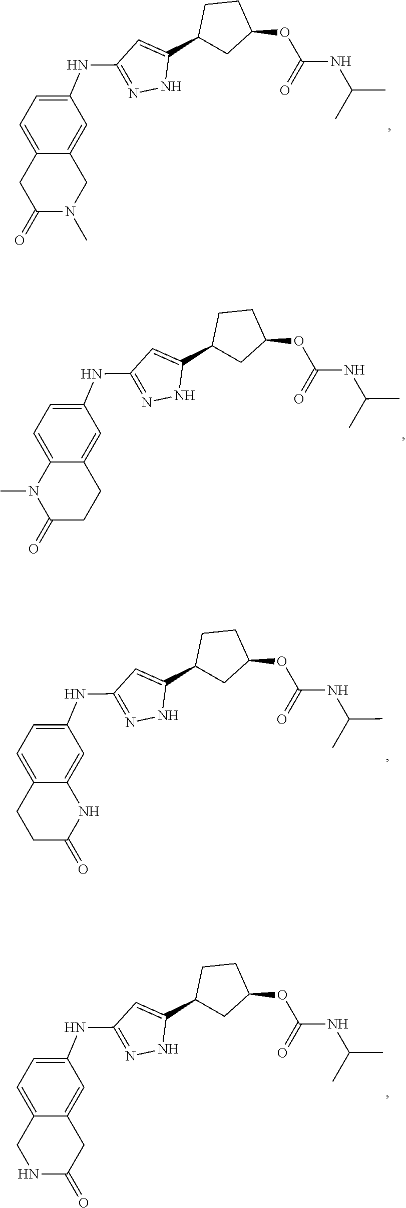 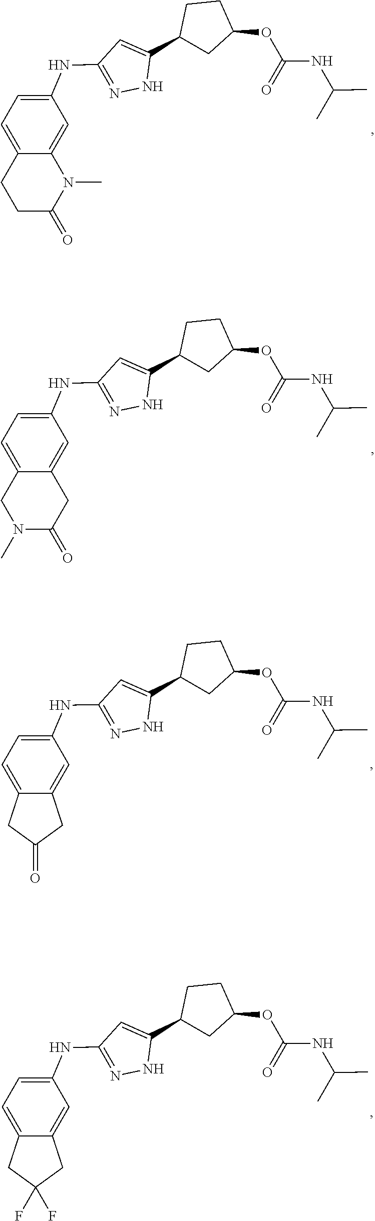 [SMILES: CC(C)NC(=O)O[C@@H]1CC[C@H](c2cc(Nc3ccc4c(c3)N(C)C(=O)CC4)n[nH]2)C1 ; CC(C)NC(=O)O[C@@H]1CC[C@H](c2cc(Nc3ccc4c(c3)CC(=O)N(C)C4)n[nH]2)C1 ; CC(C)NC(=O)O[C@@H]1CC[C@H](c2cc(Nc3ccc4c(c3)CC(=O)C4)n[nH]2)C1 ; CC(C)NC(=O)O[C@@H]1CC[C@H](c2cc(Nc3ccc4c(c3)CC(F)(F)C4)n[nH]2)C1] 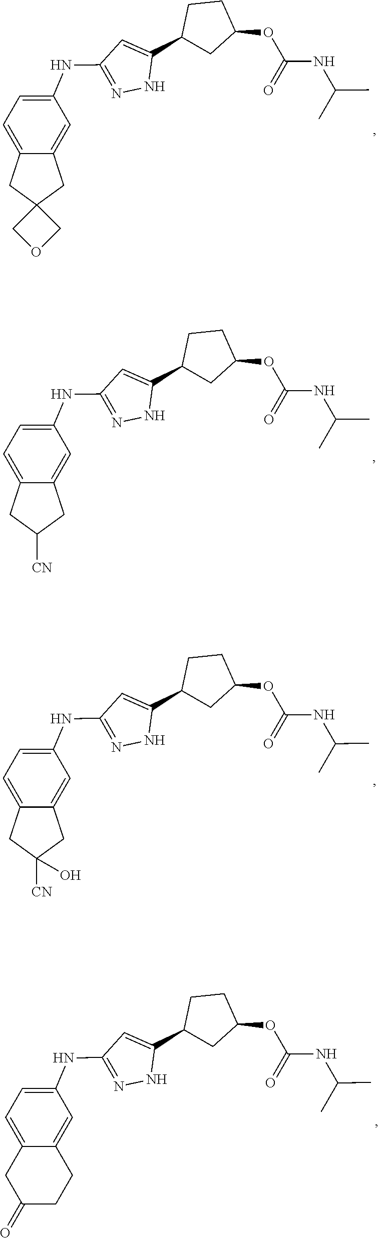 [SMILES: CC(C)NC(=O)O[C@@H]1CC[C@H](c2cc(Nc3ccc4c(c3)CC3(COC3)C4)n[nH]2)C1 ; CC(C)NC(=O)O[C@@H]1CC[C@H](c2cc(Nc3ccc4c(c3)CC(C#N)C4)n[nH]2)C1 ; CC(C)NC(=O)O[C@@H]1CC[C@H](c2cc(Nc3ccc4c(c3)CC(O)(C#N)C4)n[nH]2)C1 ; CC(C)NC(=O)O[C@@H]1CC[C@H](c2cc(Nc3ccc4c(c3)CCC(=O)C4)n[nH]2)C1] 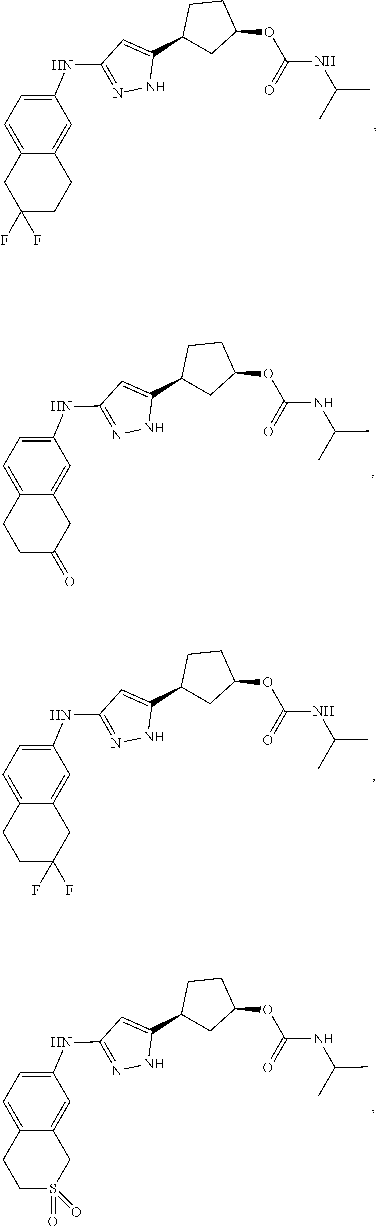 [SMILES: CC(C)NC(=O)O[C@@H]1CC[C@H](c2cc(Nc3ccc4c(c3)CCC(F)(F)C4)n[nH]2)C1 ; CC(C)NC(=O)O[C@@H]1CC[C@H](c2cc(Nc3ccc4c(c3)CC(=O)CC4)n[nH]2)C1 ; CC(C)NC(=O)O[C@@H]1CC[C@H](c2cc(Nc3ccc4c(c3)CC(F)(F)CC4)n[nH]2)C1 ; CC(C)NC(=O)O[C@@H]1CC[C@H](c2cc(Nc3ccc4c(c3)CS(=O)(=O)CC4)n[nH]2)C1] 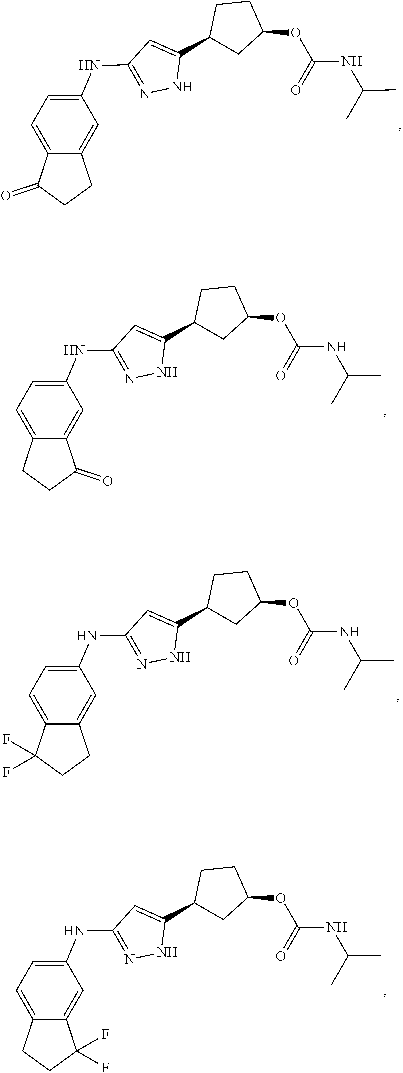 [SMILES: CC(C)NC(=O)O[C@@H]1CC[C@H](c2cc(Nc3ccc4c(c3)CCC4=O)n[nH]2)C1 ; CC(C)NC(=O)O[C@@H]1CC[C@H](c2cc(Nc3ccc4c(c3)C(=O)CC4)n[nH]2)C1 ; CC(C)NC(=O)O[C@@H]1CC[C@H](c2cc(Nc3ccc4c(c3)CCC4(F)F)n[nH]2)C1 ; CC(C)NC(=O)O[C@@H]1CC[C@H](c2cc(Nc3ccc4c(c3)C(F)(F)CC4)n[nH]2)C1] 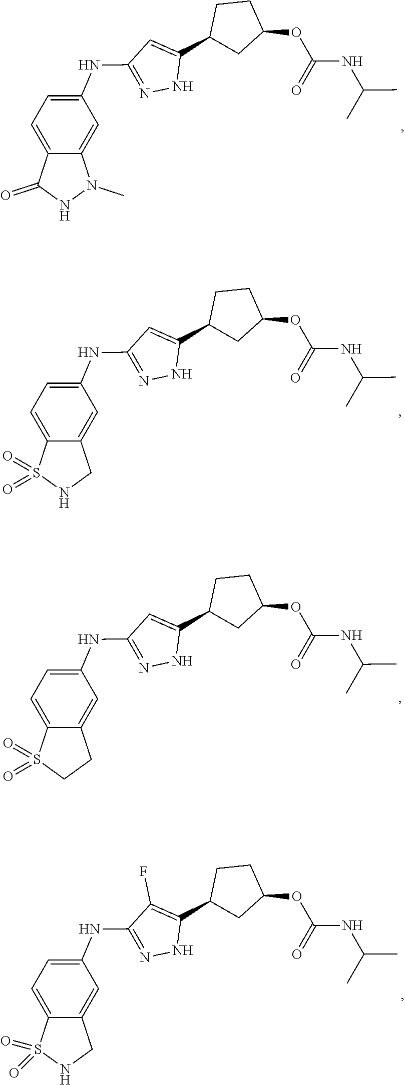 [SMILES: CC(C)NC(=O)O[C@@H]1CC[C@H](c2cc(Nc3ccc4c(=O)[nH]n(C)c4c3)n[nH]2)C1 ; CC(C)NC(=O)O[C@@H]1CC[C@H](c2cc(Nc3ccc4c(c3)CNS4(=O)=O)n[nH]2)C1 ; CC(C)NC(=O)O[C@@H]1CC[C@H](c2cc(Nc3ccc4c(c3)CCS4(=O)=O)n[nH]2)C1 ; CC(C)NC(=O)O[C@@H]1CC[C@H](c2[nH]nc(Nc3ccc4c(c3)CNS4(=O)=O)c2F)C1] 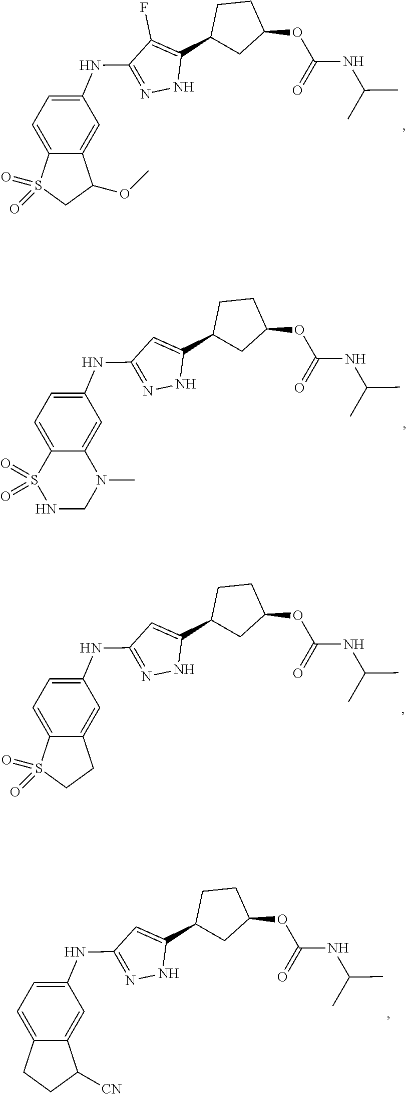 [SMILES: COC1CS(=O)(=O)c2ccc(Nc3n[nH]c([C@H]4CC[C@@H](OC(=O)NC(C)C)C4)c3F)cc21 ; CC(C)NC(=O)O[C@@H]1CC[C@H](c2cc(Nc3ccc4c(c3)N(C)CNS4(=O)=O)n[nH]2)C1 ; CC(C)NC(=O)O[C@@H]1CC[C@H](c2cc(Nc3ccc4c(c3)CCS4(=O)=O)n[nH]2)C1 ; CC(C)NC(=O)O[C@@H]1CC[C@H](c2cc(Nc3ccc4c(c3)C(C#N)CC4)n[nH]2)C1] 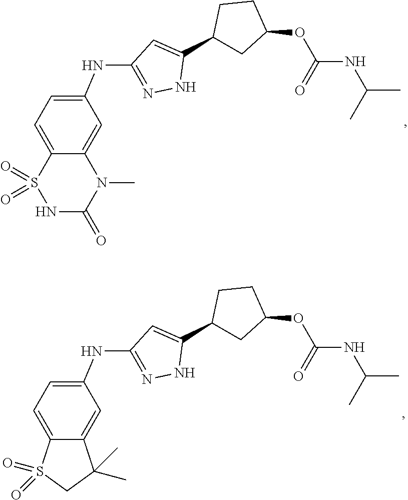 [SMILES: CC(C)NC(=O)O[C@@H]1CC[C@H](c2cc(Nc3ccc4c(c3)N(C)C(=O)NS4(=O)=O)n[nH]2)C1 ; CC(C)NC(=O)O[C@@H]1CC[C@H](c2cc(Nc3ccc4c(c3)C(C)(C)CS4(=O)=O)n[nH]2)C1] 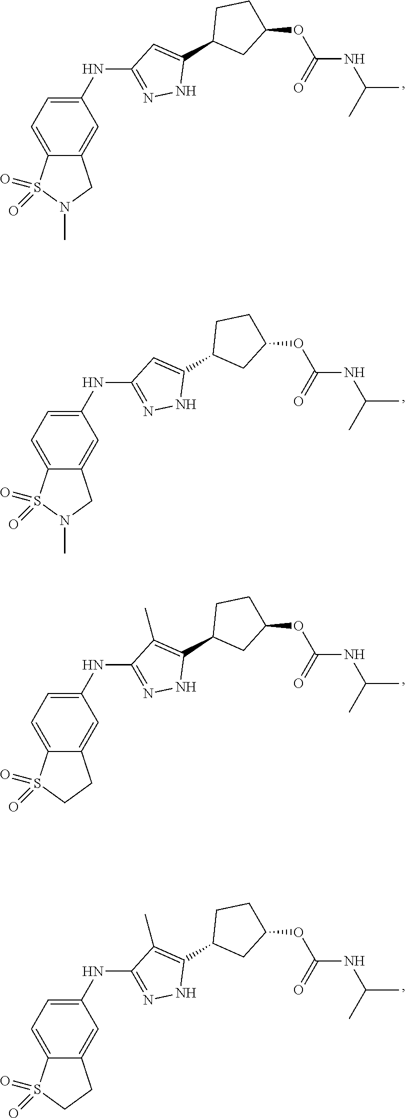 [SMILES: CC(C)NC(=O)O[C@@H]1CC[C@H](c2cc(Nc3ccc4c(c3)CN(C)S4(=O)=O)n[nH]2)C1 ; CC(C)NC(=O)O[C@H]1CC[C@@H](c2cc(Nc3ccc4c(c3)CN(C)S4(=O)=O)n[nH]2)C1 ; Cc1c(Nc2ccc3c(c2)CCS3(=O)=O)n[nH]c1[C@H]1CC[C@@H](OC(=O)NC(C)C)C1 ; Cc1c(Nc2ccc3c(c2)CCS3(=O)=O)n[nH]c1[C@@H]1CC[C@H](OC(=O)NC(C)C)C1] 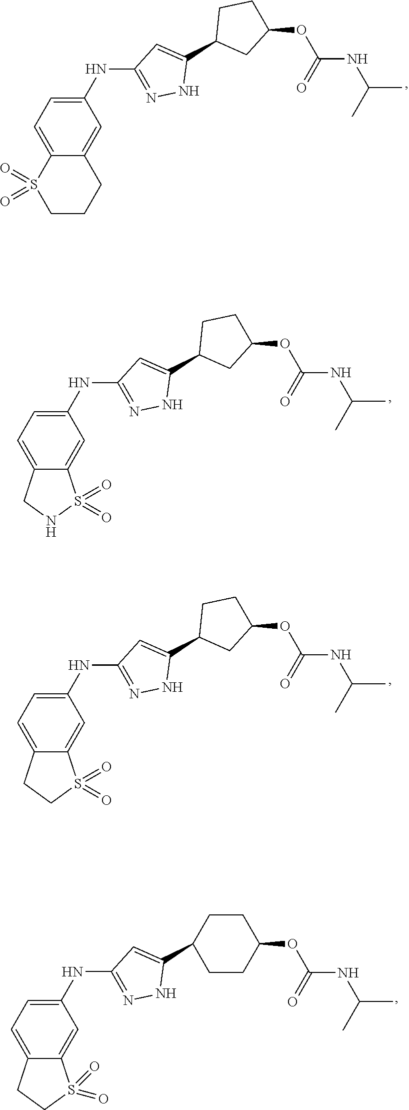 [SMILES: CC(C)NC(=O)O[C@@H]1CC[C@H](c2cc(Nc3ccc4c(c3)CCCS4(=O)=O)n[nH]2)C1 ; CC(C)NC(=O)O[C@@H]1CC[C@H](c2cc(Nc3ccc4c(c3)S(=O)(=O)NC4)n[nH]2)C1 ; CC(C)NC(=O)O[C@@H]1CC[C@H](c2cc(Nc3ccc4c(c3)S(=O)(=O)CC4)n[nH]2)C1 ; CC(C)NC(=O)O[C@H]1CC[C@@H](c2cc(Nc3ccc4c(c3)S(=O)(=O)CC4)n[nH]2)CC1] 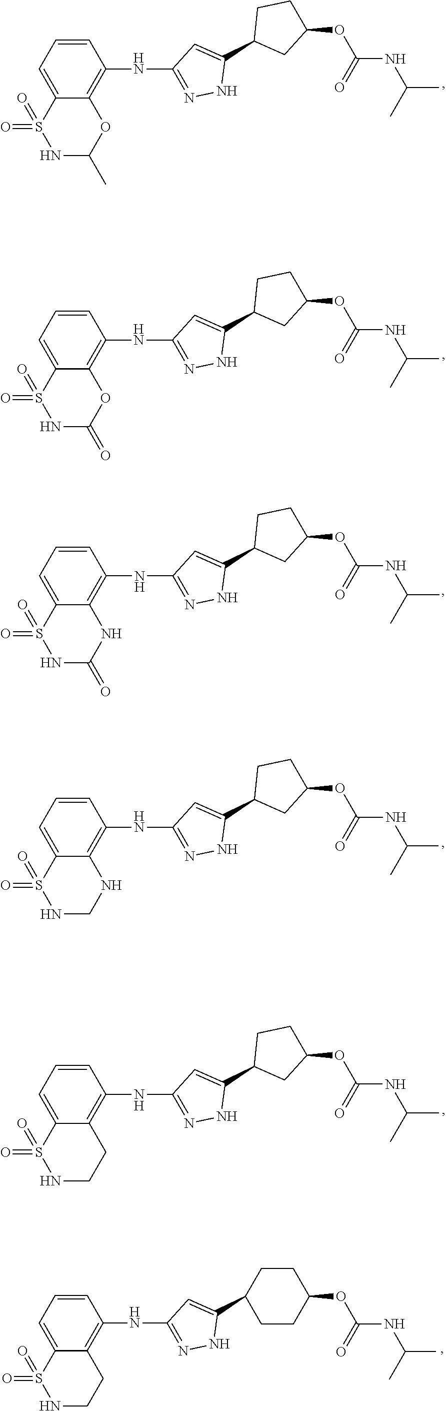 [SMILES: CC(C)NC(=O)O[C@@H]1CC[C@H](c2cc(Nc3cccc4c3OC(C)NS4(=O)=O)n[nH]2)C1 ; CC(C)NC(=O)O[C@@H]1CC[C@H](c2cc(Nc3cccc4c3OC(=O)NS4(=O)=O)n[nH]2)C1 ; CC(C)NC(=O)O[C@@H]1CC[C@H](c2cc(Nc3cccc4c3NC(=O)NS4(=O)=O)n[nH]2)C1 ; CC(C)NC(=O)O[C@@H]1CC[C@H](c2cc(Nc3cccc4c3NCNS4(=O)=O)n[nH]2)C1 ; CC(C)NC(=O)O[C@@H]1CC[C@H](c2cc(Nc3cccc4c3CCNS4(=O)=O)n[nH]2)C1 ; CC(C)NC(=O)O[C@H]1CC[C@@H](c2cc(Nc3cccc4c3CCNS4(=O)=O)n[nH]2)CC1] 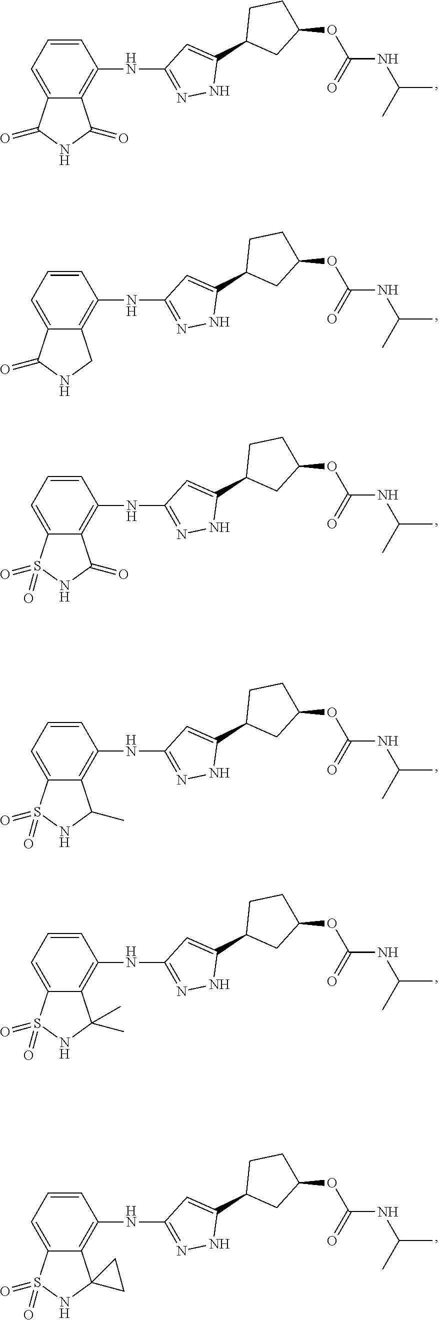 [SMILES: CC(C)NC(=O)O[C@@H]1CC[C@H](c2cc(Nc3cccc4c3C(=O)NC4=O)n[nH]2)C1 ; CC(C)NC(=O)O[C@@H]1CC[C@H](c2cc(Nc3cccc4c3CNC4=O)n[nH]2)C1 ; CC(C)NC(=O)O[C@@H]1CC[C@H](c2cc(Nc3cccc4c3C(=O)NS4(=O)=O)n[nH]2)C1 ; CC(C)NC(=O)O[C@@H]1CC[C@H](c2cc(Nc3cccc4c3C(C)NS4(=O)=O)n[nH]2)C1 ; CC(C)NC(=O)O[C@@H]1CC[C@H](c2cc(Nc3cccc4c3C(C)(C)NS4(=O)=O)n[nH]2)C1 ; CC(C)NC(=O)O[C@@H]1CC[C@H](c2cc(Nc3cccc4c3C3(CC3)NS4(=O)=O)n[nH]2)C1] 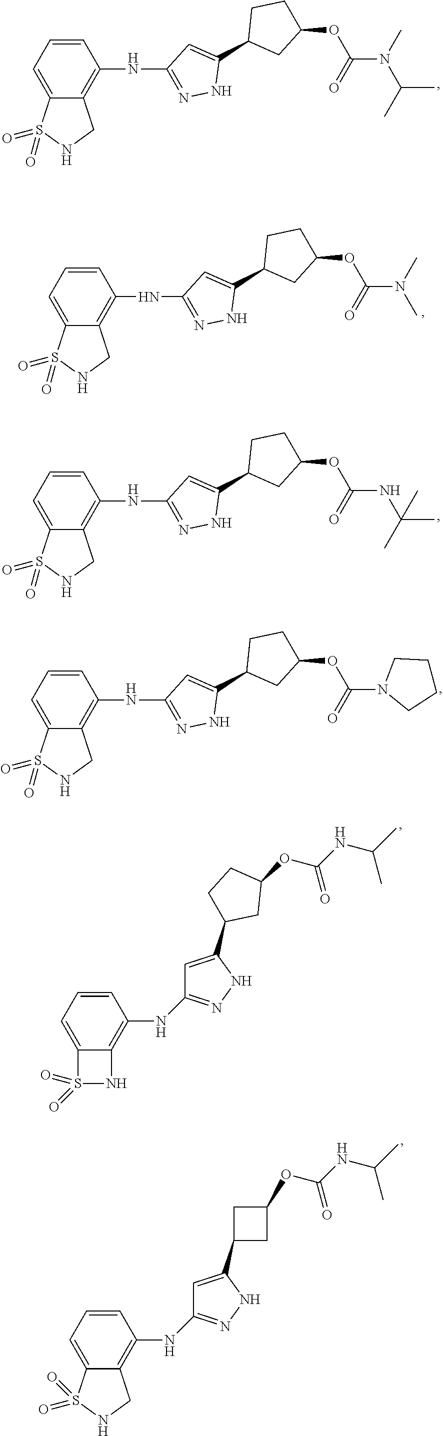 [SMILES: CC(C)N(C)C(=O)O[C@@H]1CC[C@H](c2cc(Nc3cccc4c3CNS4(=O)=O)n[nH]2)C1 ; CN(C)C(=O)O[C@@H]1CC[C@H](c2cc(Nc3cccc4c3CNS4(=O)=O)n[nH]2)C1 ; CC(C)(C)NC(=O)O[C@@H]1CC[C@H](c2cc(Nc3cccc4c3CNS4(=O)=O)n[nH]2)C1 ; O=C(O[C@@H]1CC[C@H](c2cc(Nc3cccc4c3CNS4(=O)=O)n[nH]2)C1)N1CCCC1 ; CC(C)NC(=O)O[C@@H]1CC[C@H](c2cc(Nc3cccc4c3NS4(=O)=O)n[nH]2)C1 ; CC(C)NC(=O)O[C@H]1C[C@@H](c2cc(Nc3cccc4c3CNS4(=O)=O)n[nH]2)C1] 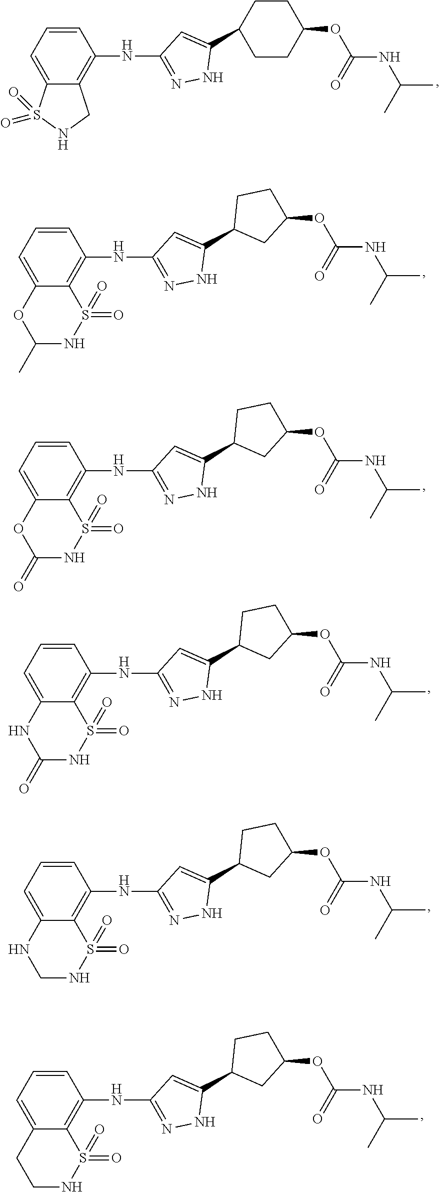 [SMILES: CC(C)NC(=O)O[C@H]1CC[C@@H](c2cc(Nc3cccc4c3CNS4(=O)=O)n[nH]2)CC1 ; CC(C)NC(=O)O[C@@H]1CC[C@H](c2cc(Nc3cccc4c3S(=O)(=O)NC(C)O4)n[nH]2)C1 ; CC(C)NC(=O)O[C@@H]1CC[C@H](c2cc(Nc3cccc4c3S(=O)(=O)NC(=O)O4)n[nH]2)C1 ; CC(C)NC(=O)O[C@@H]1CC[C@H](c2cc(Nc3cccc4c3S(=O)(=O)NC(=O)N4)n[nH]2)C1 ; CC(C)NC(=O)O[C@@H]1CC[C@H](c2cc(Nc3cccc4c3S(=O)(=O)NCN4)n[nH]2)C1 ; CC(C)NC(=O)O[C@@H]1CC[C@H](c2cc(Nc3cccc4c3S(=O)(=O)NCC4)n[nH]2)C1] 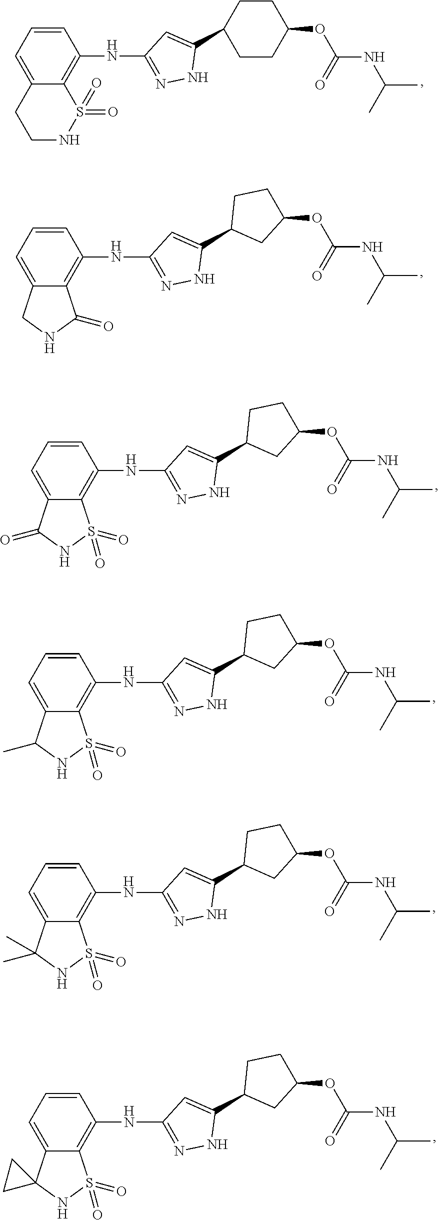 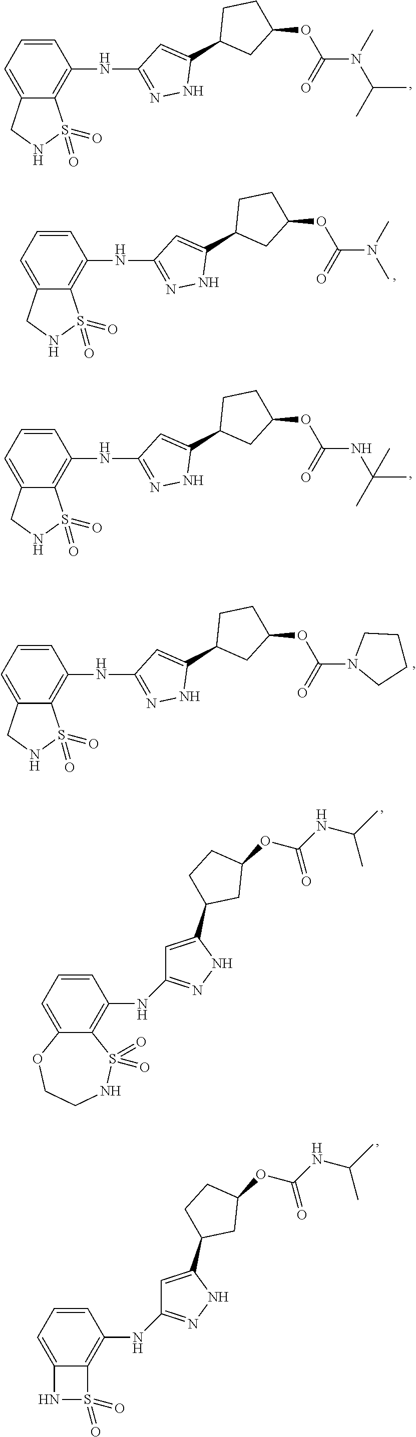 [SMILES: CC(C)N(C)C(=O)O[C@@H]1CC[C@H](c2cc(Nc3cccc4c3S(=O)(=O)NC4)n[nH]2)C1 ; CN(C)C(=O)O[C@@H]1CC[C@H](c2cc(Nc3cccc4c3S(=O)(=O)NC4)n[nH]2)C1 ; CC(C)(C)NC(=O)O[C@@H]1CC[C@H](c2cc(Nc3cccc4c3S(=O)(=O)NC4)n[nH]2)C1 ; O=C(O[C@@H]1CC[C@H](c2cc(Nc3cccc4c3S(=O)(=O)NC4)n[nH]2)C1)N1CCCC1 ; CC(C)NC(=O)O[C@@H]1CC[C@H](c2cc(Nc3cccc4c3S(=O)(=O)NCCO4)n[nH]2)C1 ; CC(C)NC(=O)O[C@@H]1CC[C@H](c2cc(Nc3cccc4c3S(=O)(=O)N4)n[nH]2)C1] 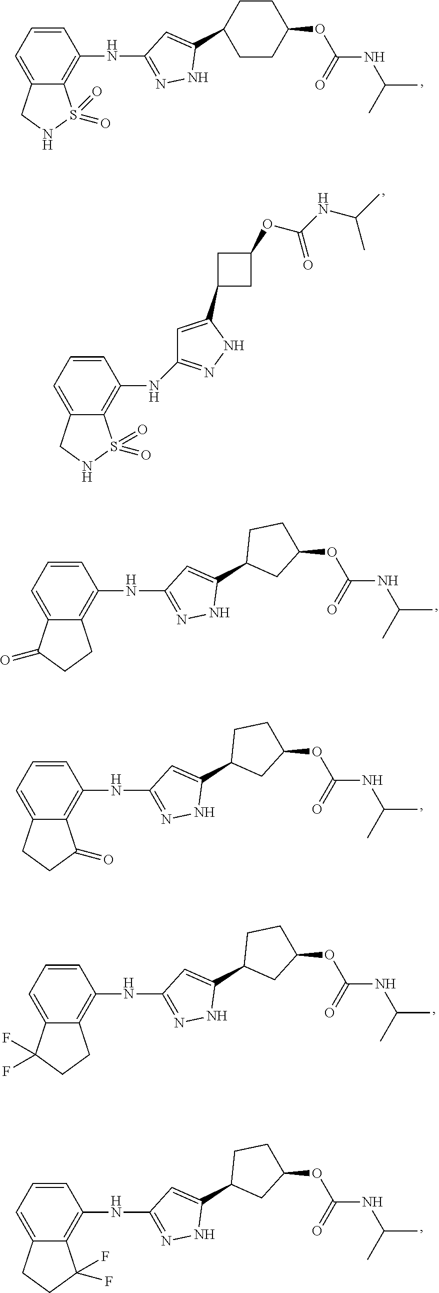 [SMILES: CC(C)NC(=O)O[C@H]1CC[C@@H](c2cc(Nc3cccc4c3S(=O)(=O)NC4)n[nH]2)CC1 ; CC(C)NC(=O)O[C@H]1C[C@@H](c2cc(Nc3cccc4c3S(=O)(=O)NC4)n[nH]2)C1 ; CC(C)NC(=O)O[C@@H]1CC[C@H](c2cc(Nc3cccc4c3CCC4=O)n[nH]2)C1 ; CC(C)NC(=O)O[C@@H]1CC[C@H](c2cc(Nc3cccc4c3C(=O)CC4)n[nH]2)C1 ; CC(C)NC(=O)O[C@@H]1CC[C@H](c2cc(Nc3cccc4c3CCC4(F)F)n[nH]2)C1 ; CC(C)NC(=O)O[C@@H]1CC[C@H](c2cc(Nc3cccc4c3C(F)(F)CC4)n[nH]2)C1] 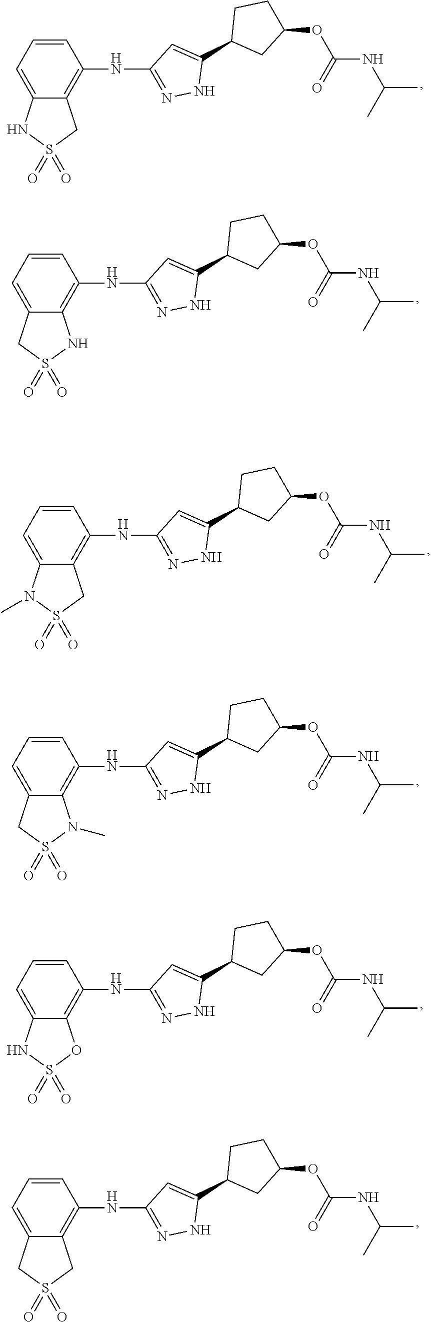 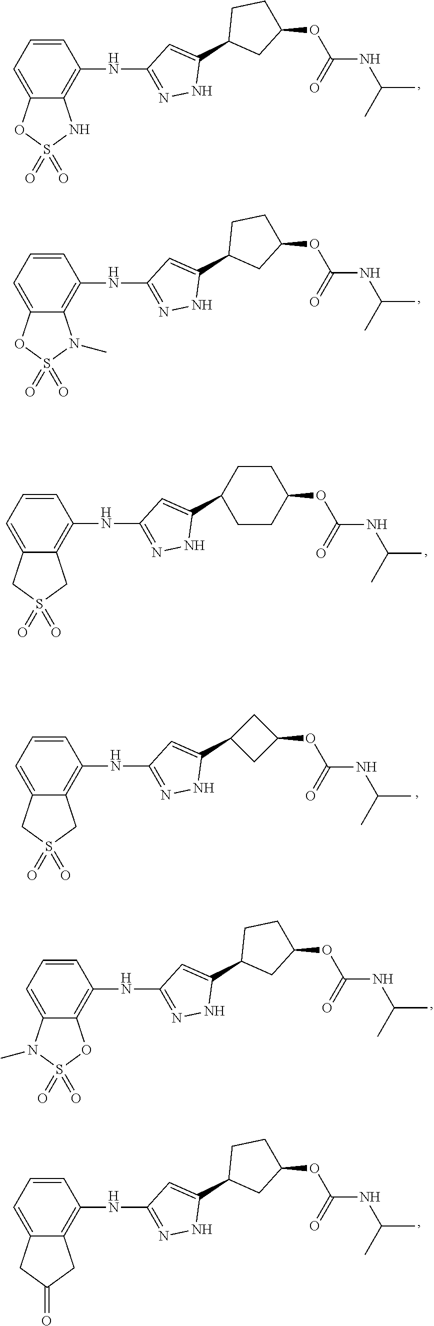 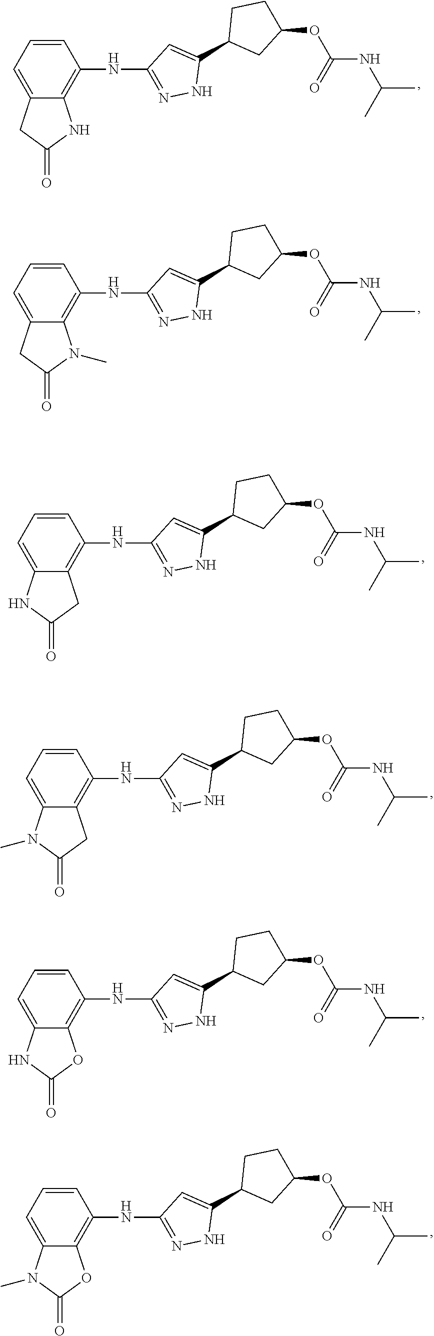 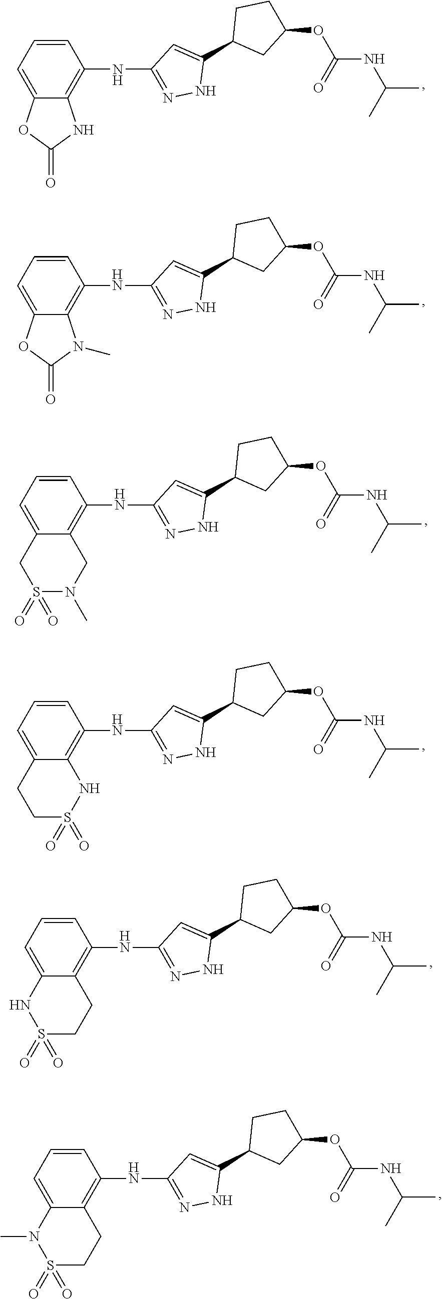 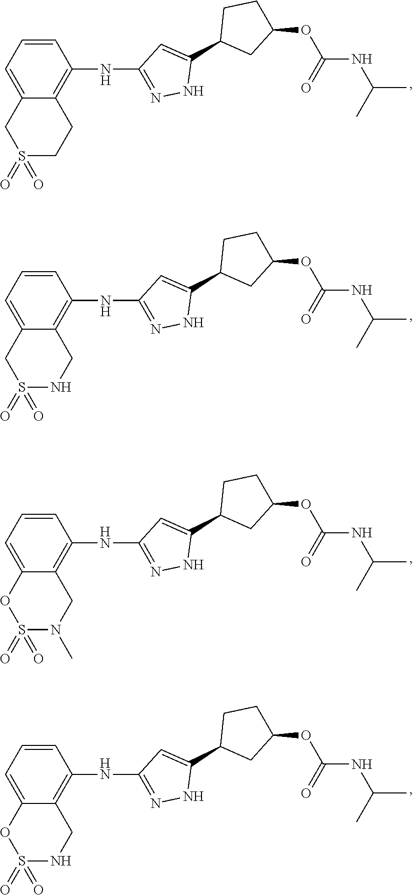 [SMILES: CC(C)NC(=O)O[C@@H]1CC[C@H](c2cc(Nc3cccc4c3CCS(=O)(=O)C4)n[nH]2)C1 ; CC(C)NC(=O)O[C@@H]1CC[C@H](c2cc(Nc3cccc4c3CNS(=O)(=O)C4)n[nH]2)C1 ; CC(C)NC(=O)O[C@@H]1CC[C@H](c2cc(Nc3cccc4c3CN(C)S(=O)(=O)O4)n[nH]2)C1 ; CC(C)NC(=O)O[C@@H]1CC[C@H](c2cc(Nc3cccc4c3CNS(=O)(=O)O4)n[nH]2)C1] 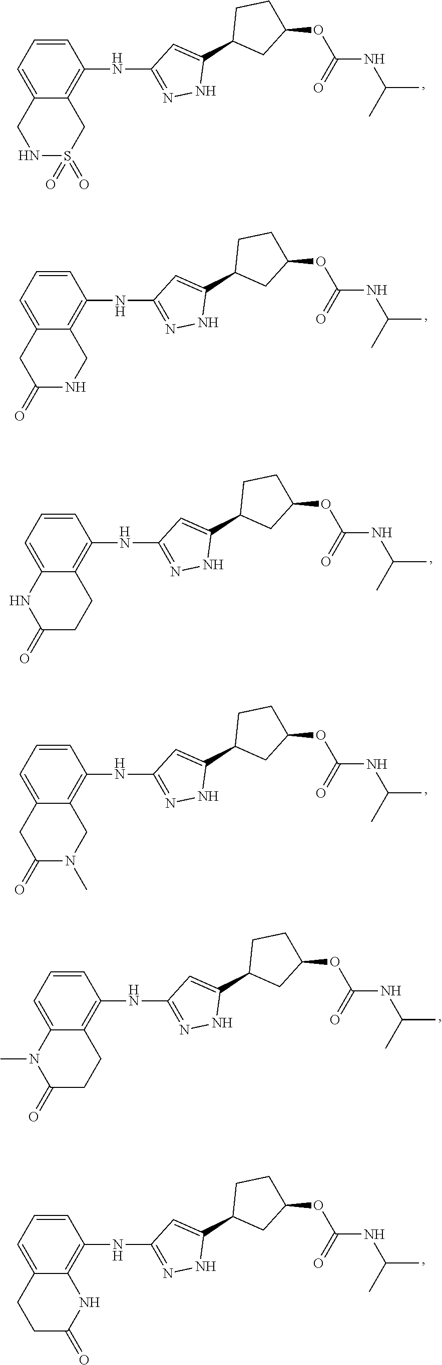 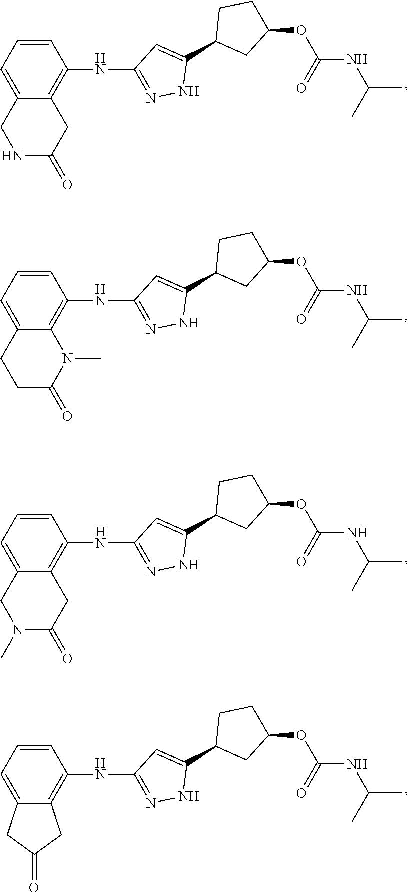 [SMILES: CC(C)NC(=O)O[C@@H]1CC[C@H](c2cc(Nc3cccc4c3CC(=O)NC4)n[nH]2)C1 ; CC(C)NC(=O)O[C@@H]1CC[C@H](c2cc(Nc3cccc4c3N(C)C(=O)CC4)n[nH]2)C1 ; CC(C)NC(=O)O[C@@H]1CC[C@H](c2cc(Nc3cccc4c3CC(=O)N(C)C4)n[nH]2)C1 ; CC(C)NC(=O)O[C@@H]1CC[C@H](c2cc(Nc3cccc4c3CC(=O)C4)n[nH]2)C1] 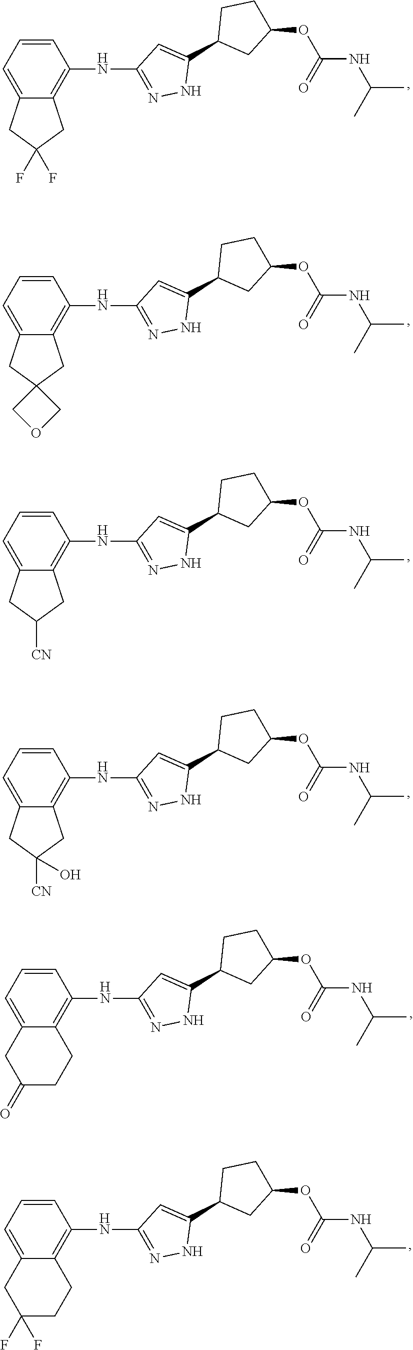 [SMILES: CC(C)NC(=O)O[C@@H]1CC[C@H](c2cc(Nc3cccc4c3CC(F)(F)C4)n[nH]2)C1 ; CC(C)NC(=O)O[C@@H]1CC[C@H](c2cc(Nc3cccc4c3CC3(COC3)C4)n[nH]2)C1 ; CC(C)NC(=O)O[C@@H]1CC[C@H](c2cc(Nc3cccc4c3CC(C#N)C4)n[nH]2)C1 ; CC(C)NC(=O)O[C@@H]1CC[C@H](c2cc(Nc3cccc4c3CC(O)(C#N)C4)n[nH]2)C1 ; CC(C)NC(=O)O[C@@H]1CC[C@H](c2cc(Nc3cccc4c3CCC(=O)C4)n[nH]2)C1 ; CC(C)NC(=O)O[C@@H]1CC[C@H](c2cc(Nc3cccc4c3CCC(F)(F)C4)n[nH]2)C1] 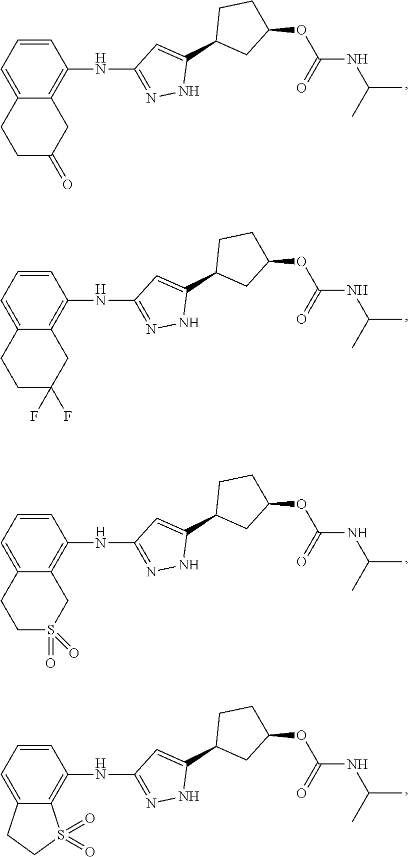 [SMILES: CC(C)NC(=O)O[C@@H]1CC[C@H](c2cc(Nc3cccc4c3CC(=O)CC4)n[nH]2)C1 ; CC(C)NC(=O)O[C@@H]1CC[C@H](c2cc(Nc3cccc4c3CC(F)(F)CC4)n[nH]2)C1 ; CC(C)NC(=O)O[C@@H]1CC[C@H](c2cc(Nc3cccc4c3CS(=O)(=O)CC4)n[nH]2)C1 ; CC(C)NC(=O)O[C@@H]1CC[C@H](c2cc(Nc3cccc4c3S(=O)(=O)CC4)n[nH]2)C1] 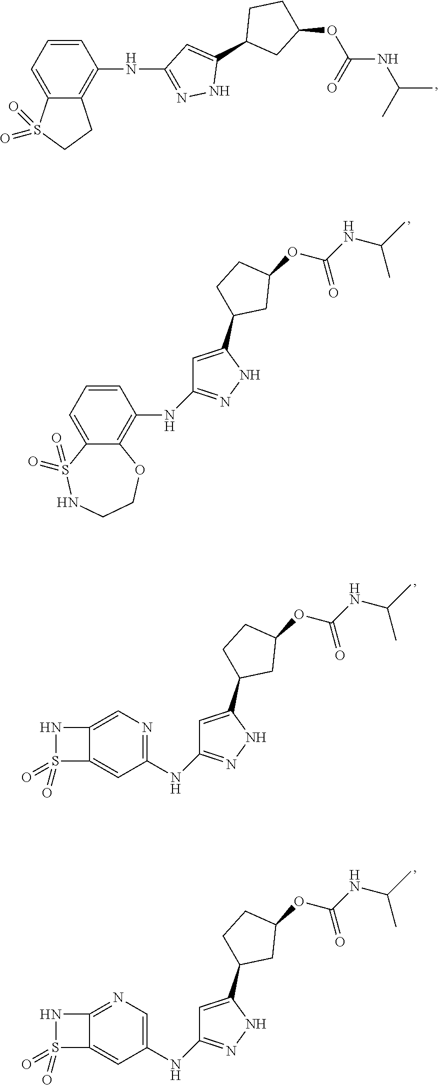 [SMILES: CC(C)NC(=O)O[C@@H]1CC[C@H](c2cc(Nc3cccc4c3CCS4(=O)=O)n[nH]2)C1 ; CC(C)NC(=O)O[C@@H]1CC[C@H](c2cc(Nc3cccc4c3OCCNS4(=O)=O)n[nH]2)C1 ; CC(C)NC(=O)O[C@@H]1CC[C@H](c2cc(Nc3cc4c(cn3)NS4(=O)=O)n[nH]2)C1 ; CC(C)NC(=O)O[C@@H]1CC[C@H](c2cc(Nc3cnc4c(c3)S(=O)(=O)N4)n[nH]2)C1] 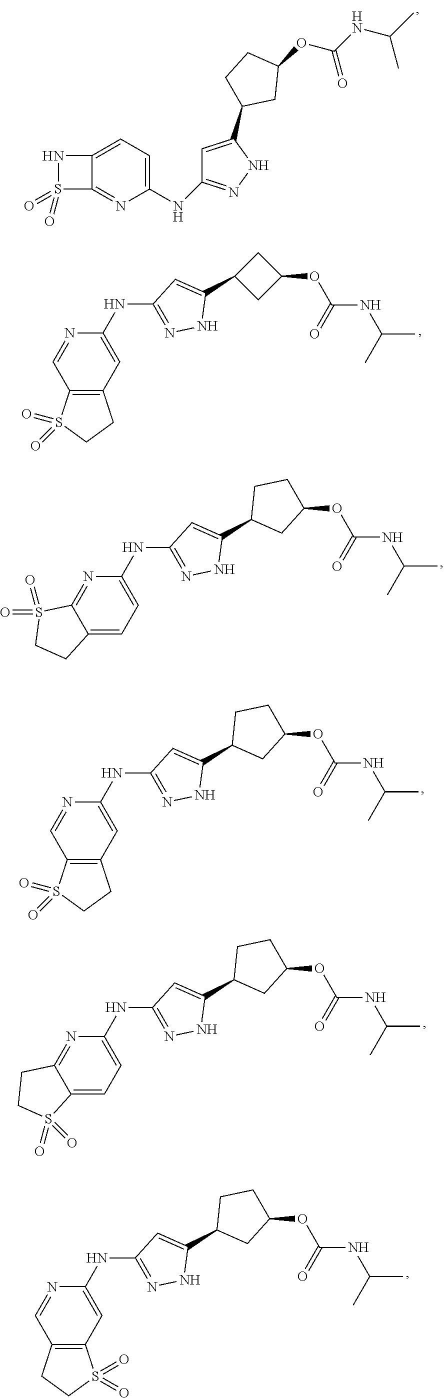 [SMILES: CC(C)NC(=O)O[C@@H]1CC[C@H](c2cc(Nc3ccc4c(n3)S(=O)(=O)N4)n[nH]2)C1 ; CC(C)NC(=O)O[C@H]1C[C@@H](c2cc(Nc3cc4c(cn3)S(=O)(=O)CC4)n[nH]2)C1 ; CC(C)NC(=O)O[C@@H]1CC[C@H](c2cc(Nc3ccc4c(n3)S(=O)(=O)CC4)n[nH]2)C1 ; CC(C)NC(=O)O[C@@H]1CC[C@H](c2cc(Nc3cc4c(cn3)S(=O)(=O)CC4)n[nH]2)C1 ; CC(C)NC(=O)O[C@@H]1CC[C@H](c2cc(Nc3ccc4c(n3)CCS4(=O)=O)n[nH]2)C1 ; CC(C)NC(=O)O[C@@H]1CC[C@H](c2cc(Nc3cc4c(cn3)CCS4(=O)=O)n[nH]2)C1] 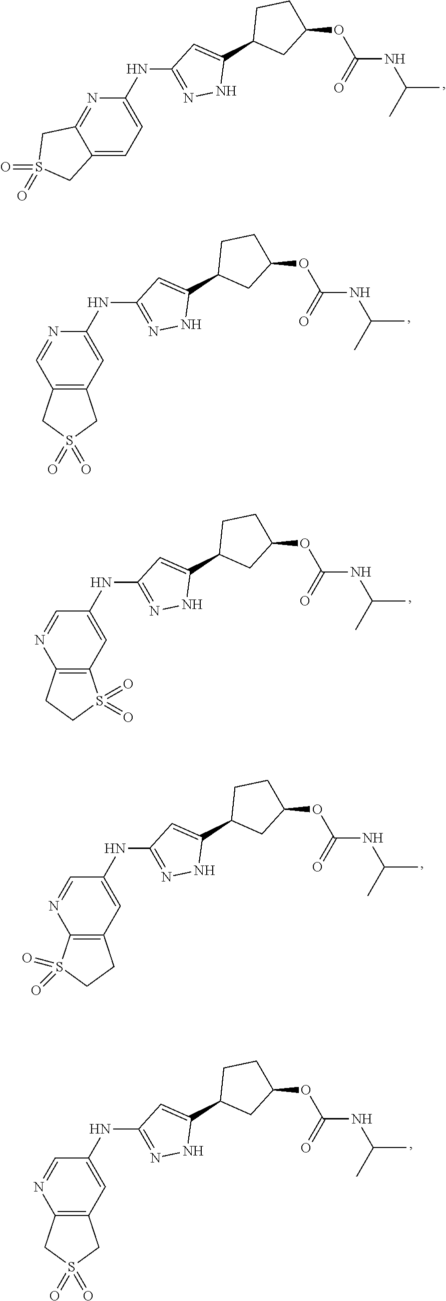 [SMILES: CC(C)NC(=O)O[C@@H]1CC[C@H](c2cc(Nc3ccc4c(n3)CS(=O)(=O)C4)n[nH]2)C1 ; CC(C)NC(=O)O[C@@H]1CC[C@H](c2cc(Nc3cc4c(cn3)CS(=O)(=O)C4)n[nH]2)C1 ; CC(C)NC(=O)O[C@@H]1CC[C@H](c2cc(Nc3cnc4c(c3)S(=O)(=O)CC4)n[nH]2)C1 ; CC(C)NC(=O)O[C@@H]1CC[C@H](c2cc(Nc3cnc4c(c3)CCS4(=O)=O)n[nH]2)C1 ; CC(C)NC(=O)O[C@@H]1CC[C@H](c2cc(Nc3cnc4c(c3)CS(=O)(=O)C4)n[nH]2)C1] 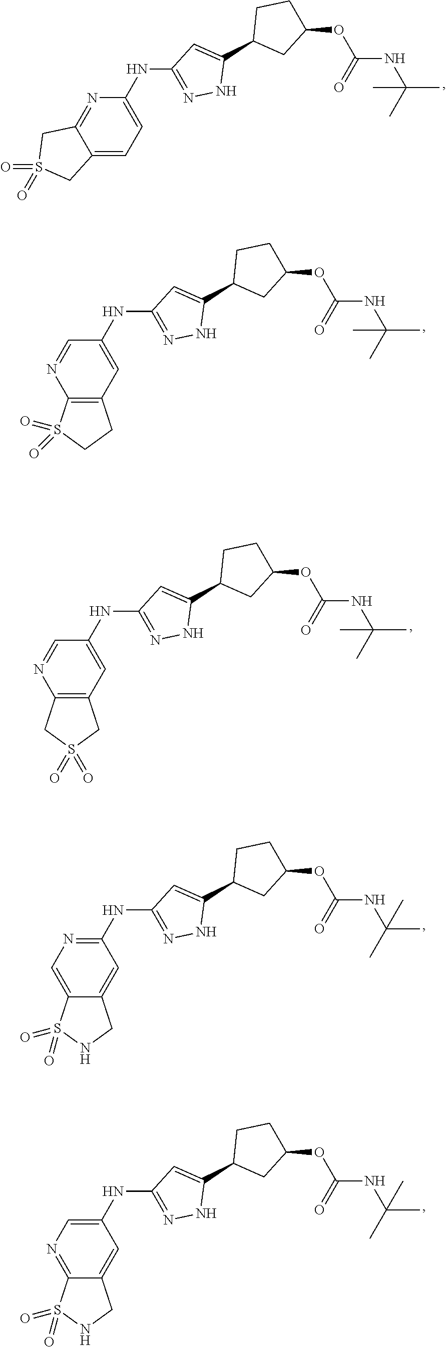 [SMILES: CC(C)(C)NC(=O)O[C@@H]1CC[C@H](c2cc(Nc3ccc4c(n3)CS(=O)(=O)C4)n[nH]2)C1 ; CC(C)(C)NC(=O)O[C@@H]1CC[C@H](c2cc(Nc3cnc4c(c3)CCS4(=O)=O)n[nH]2)C1 ; CC(C)(C)NC(=O)O[C@@H]1CC[C@H](c2cc(Nc3cnc4c(c3)CS(=O)(=O)C4)n[nH]2)C1 ; CC(C)(C)NC(=O)O[C@@H]1CC[C@H](c2cc(Nc3cc4c(cn3)S(=O)(=O)NC4)n[nH]2)C1 ; CC(C)(C)NC(=O)O[C@@H]1CC[C@H](c2cc(Nc3cnc4c(c3)CNS4(=O)=O)n[nH]2)C1] 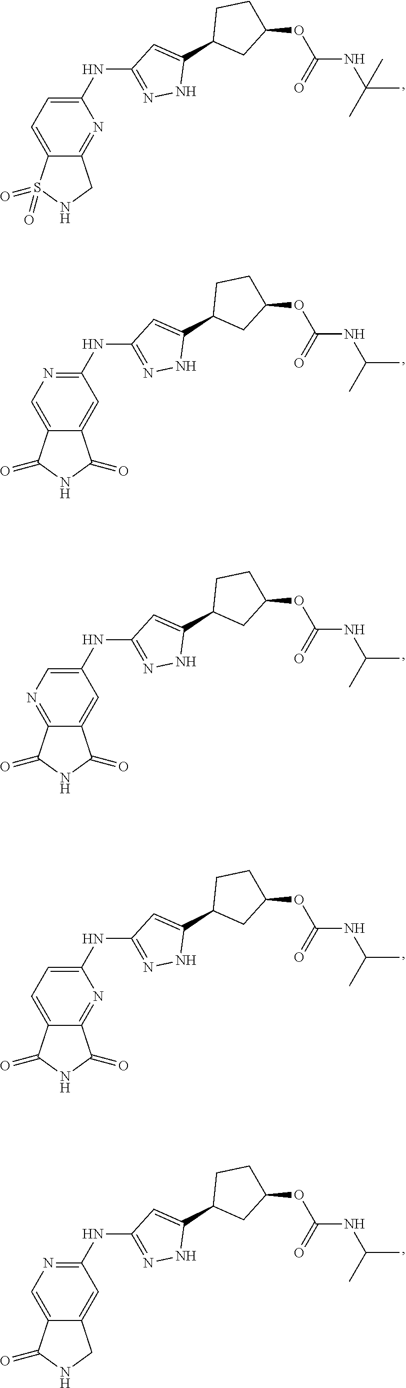 [SMILES: CC(C)(C)NC(=O)O[C@@H]1CC[C@H](c2cc(Nc3ccc4c(n3)CNS4(=O)=O)n[nH]2)C1 ; CC(C)NC(=O)O[C@@H]1CC[C@H](c2cc(Nc3cc4c(cn3)C(=O)NC4=O)n[nH]2)C1 ; CC(C)NC(=O)O[C@@H]1CC[C@H](c2cc(Nc3cnc4c(c3)C(=O)NC4=O)n[nH]2)C1 ; CC(C)NC(=O)O[C@@H]1CC[C@H](c2cc(Nc3ccc4c(n3)C(=O)NC4=O)n[nH]2)C1 ; CC(C)NC(=O)O[C@@H]1CC[C@H](c2cc(Nc3cc4c(cn3)C(=O)NC4)n[nH]2)C1] 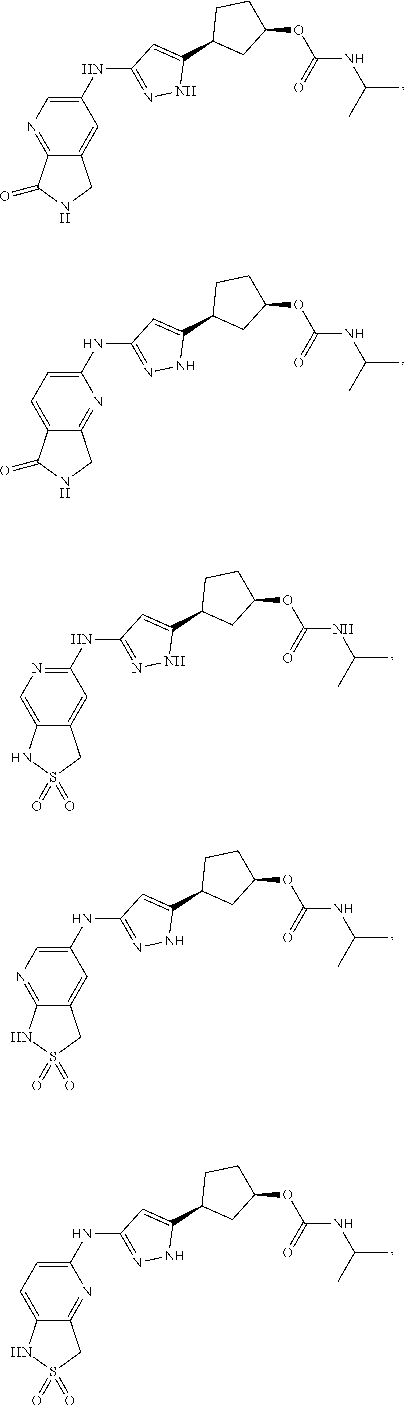 [SMILES: CC(C)NC(=O)O[C@@H]1CC[C@H](c2cc(Nc3cnc4c(c3)CNC4=O)n[nH]2)C1 ; CC(C)NC(=O)O[C@@H]1CC[C@H](c2cc(Nc3ccc4c(n3)CNC4=O)n[nH]2)C1 ; CC(C)NC(=O)O[C@@H]1CC[C@H](c2cc(Nc3cc4c(cn3)NS(=O)(=O)C4)n[nH]2)C1 ; CC(C)NC(=O)O[C@@H]1CC[C@H](c2cc(Nc3cnc4c(c3)CS(=O)(=O)N4)n[nH]2)C1 ; CC(C)NC(=O)O[C@@H]1CC[C@H](c2cc(Nc3ccc4c(n3)CS(=O)(=O)N4)n[nH]2)C1] 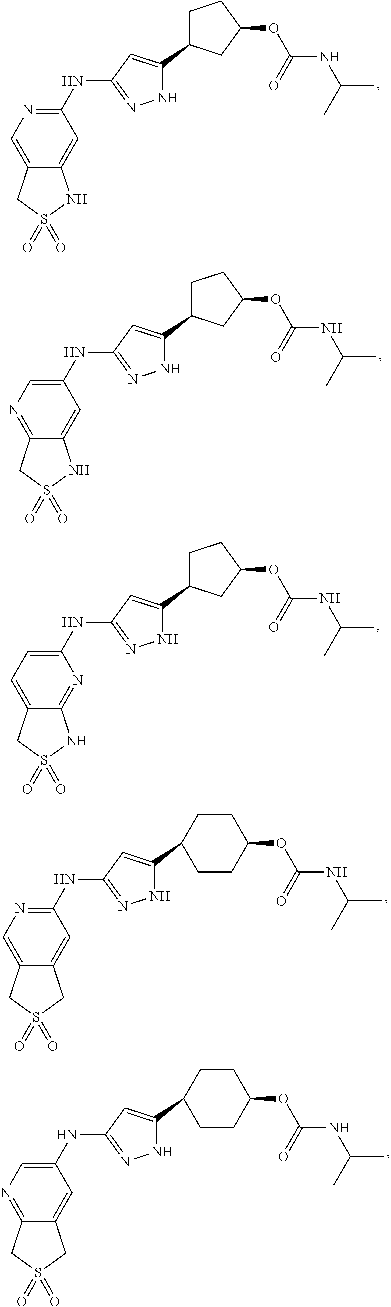 [SMILES: CC(C)NC(=O)O[C@@H]1CC[C@H](c2cc(Nc3cc4c(cn3)CS(=O)(=O)N4)n[nH]2)C1 ; CC(C)NC(=O)O[C@@H]1CC[C@H](c2cc(Nc3cnc4c(c3)NS(=O)(=O)C4)n[nH]2)C1 ; CC(C)NC(=O)O[C@@H]1CC[C@H](c2cc(Nc3ccc4c(n3)NS(=O)(=O)C4)n[nH]2)C1 ; CC(C)NC(=O)O[C@H]1CC[C@@H](c2cc(Nc3cc4c(cn3)CS(=O)(=O)C4)n[nH]2)CC1 ; CC(C)NC(=O)O[C@H]1CC[C@@H](c2cc(Nc3cnc4c(c3)CS(=O)(=O)C4)n[nH]2)CC1] 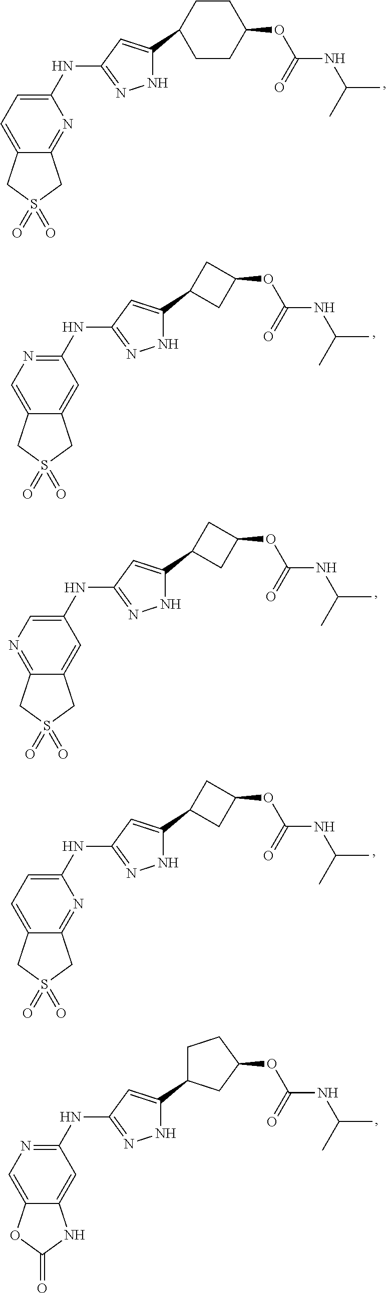 [SMILES: CC(C)NC(=O)O[C@H]1CC[C@@H](c2cc(Nc3ccc4c(n3)CS(=O)(=O)C4)n[nH]2)CC1 ; CC(C)NC(=O)O[C@H]1C[C@@H](c2cc(Nc3cc4c(cn3)CS(=O)(=O)C4)n[nH]2)C1 ; CC(C)NC(=O)O[C@H]1C[C@@H](c2cc(Nc3cnc4c(c3)CS(=O)(=O)C4)n[nH]2)C1 ; CC(C)NC(=O)O[C@H]1C[C@@H](c2cc(Nc3ccc4c(n3)CS(=O)(=O)C4)n[nH]2)C1 ; CC(C)NC(=O)O[C@@H]1CC[C@H](c2cc(Nc3cc4[nH]c(=O)oc4cn3)n[nH]2)C1] 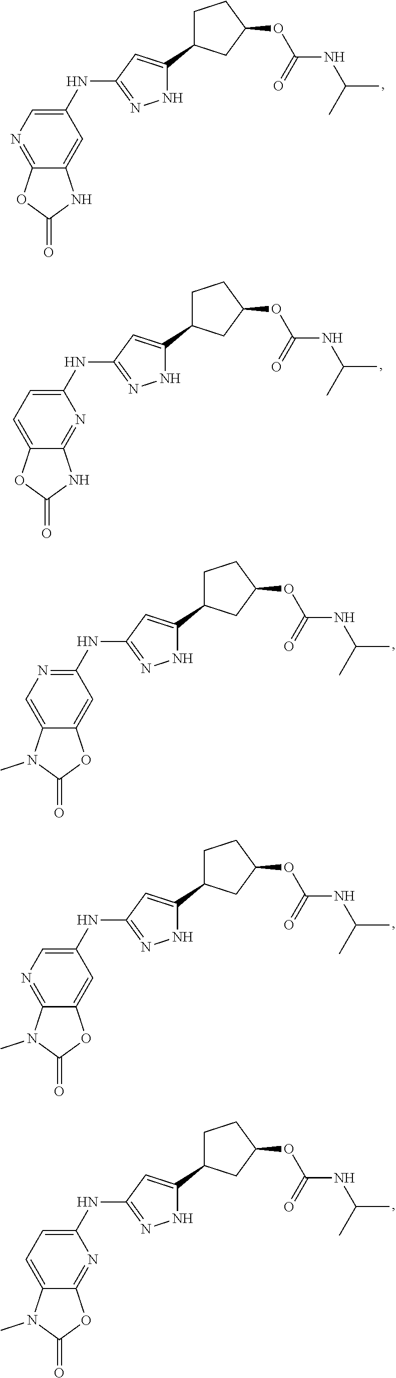 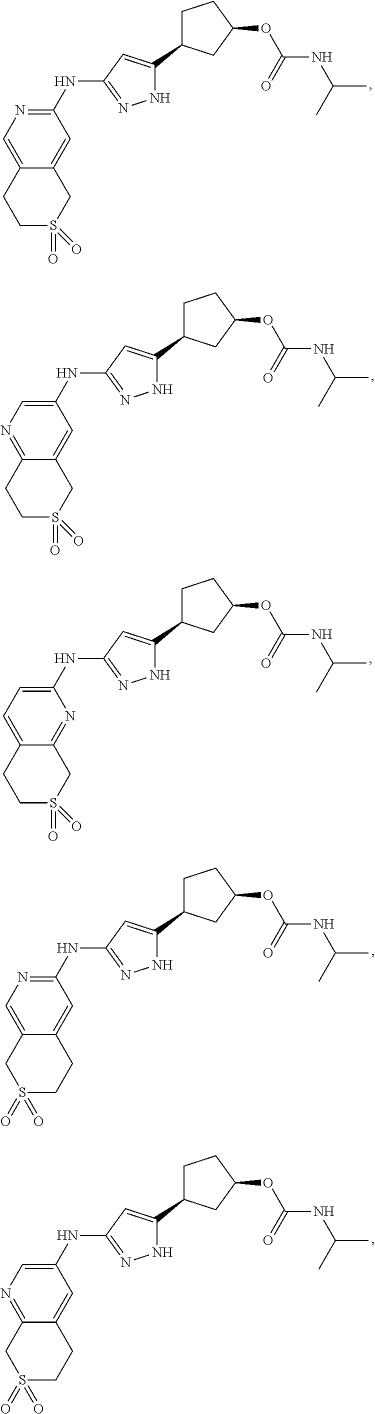 [SMILES: CC(C)NC(=O)O[C@@H]1CC[C@H](c2cc(Nc3cc4c(cn3)CCS(=O)(=O)C4)n[nH]2)C1 ; CC(C)NC(=O)O[C@@H]1CC[C@H](c2cc(Nc3cnc4c(c3)CS(=O)(=O)CC4)n[nH]2)C1 ; CC(C)NC(=O)O[C@@H]1CC[C@H](c2cc(Nc3ccc4c(n3)CS(=O)(=O)CC4)n[nH]2)C1 ; CC(C)NC(=O)O[C@@H]1CC[C@H](c2cc(Nc3cc4c(cn3)CS(=O)(=O)CC4)n[nH]2)C1 ; CC(C)NC(=O)O[C@@H]1CC[C@H](c2cc(Nc3cnc4c(c3)CCS(=O)(=O)C4)n[nH]2)C1] 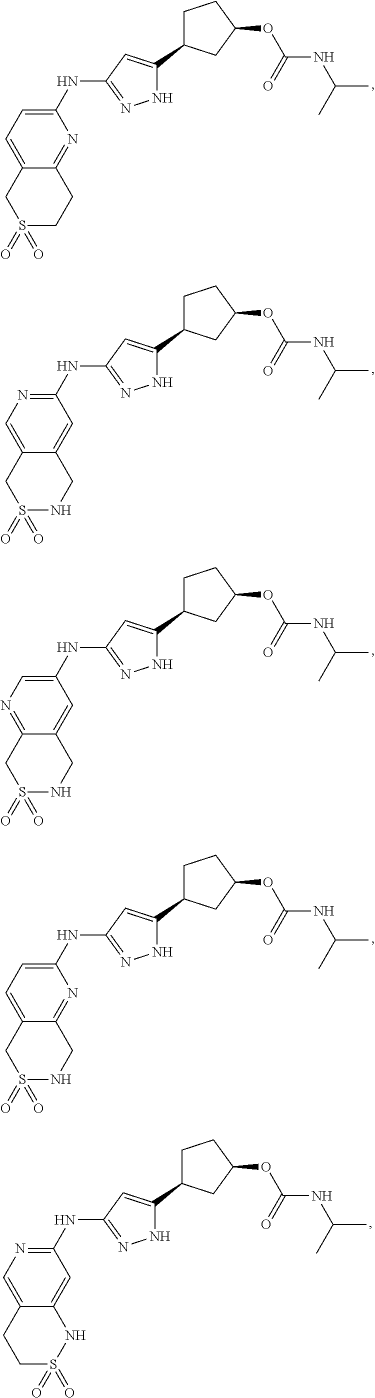 [SMILES: CC(C)NC(=O)O[C@@H]1CC[C@H](c2cc(Nc3ccc4c(n3)CCS(=O)(=O)C4)n[nH]2)C1 ; CC(C)NC(=O)O[C@@H]1CC[C@H](c2cc(Nc3cc4c(cn3)CS(=O)(=O)NC4)n[nH]2)C1 ; CC(C)NC(=O)O[C@@H]1CC[C@H](c2cc(Nc3cnc4c(c3)CNS(=O)(=O)C4)n[nH]2)C1 ; CC(C)NC(=O)O[C@@H]1CC[C@H](c2cc(Nc3ccc4c(n3)CNS(=O)(=O)C4)n[nH]2)C1 ; CC(C)NC(=O)O[C@@H]1CC[C@H](c2cc(Nc3cc4c(cn3)CCS(=O)(=O)N4)n[nH]2)C1] 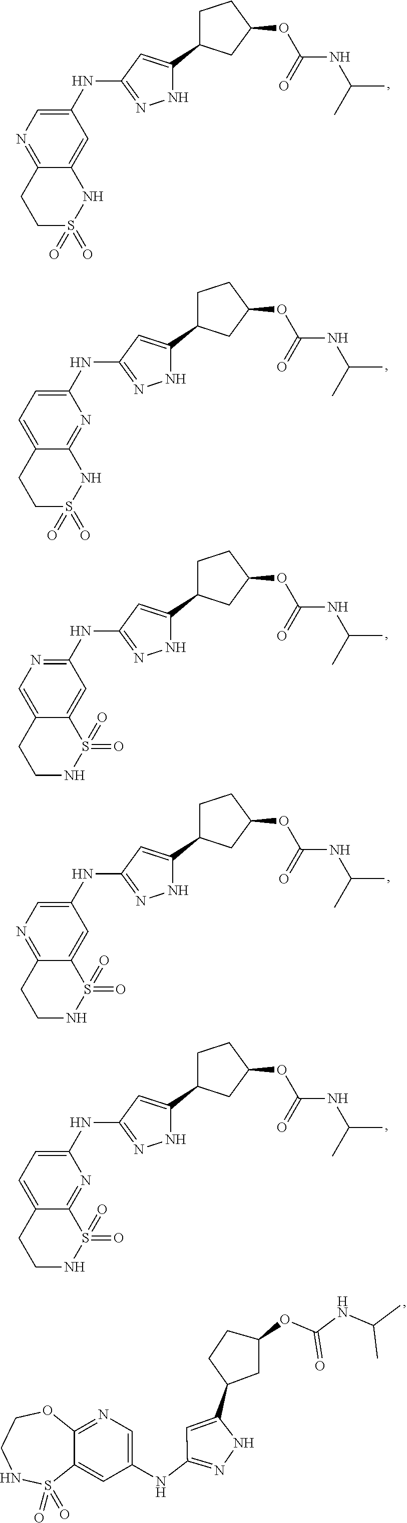 [SMILES: CC(C)NC(=O)O[C@@H]1CC[C@H](c2cc(Nc3cnc4c(c3)NS(=O)(=O)CC4)n[nH]2)C1 ; CC(C)NC(=O)O[C@@H]1CC[C@H](c2cc(Nc3ccc4c(n3)NS(=O)(=O)CC4)n[nH]2)C1 ; CC(C)NC(=O)O[C@@H]1CC[C@H](c2cc(Nc3cc4c(cn3)CCNS4(=O)=O)n[nH]2)C1 ; CC(C)NC(=O)O[C@@H]1CC[C@H](c2cc(Nc3cnc4c(c3)S(=O)(=O)NCC4)n[nH]2)C1 ; CC(C)NC(=O)O[C@@H]1CC[C@H](c2cc(Nc3ccc4c(n3)S(=O)(=O)NCC4)n[nH]2)C1 ; CC(C)NC(=O)O[C@@H]1CC[C@H](c2cc(Nc3cnc4c(c3)S(=O)(=O)NCCO4)n[nH]2)C1] 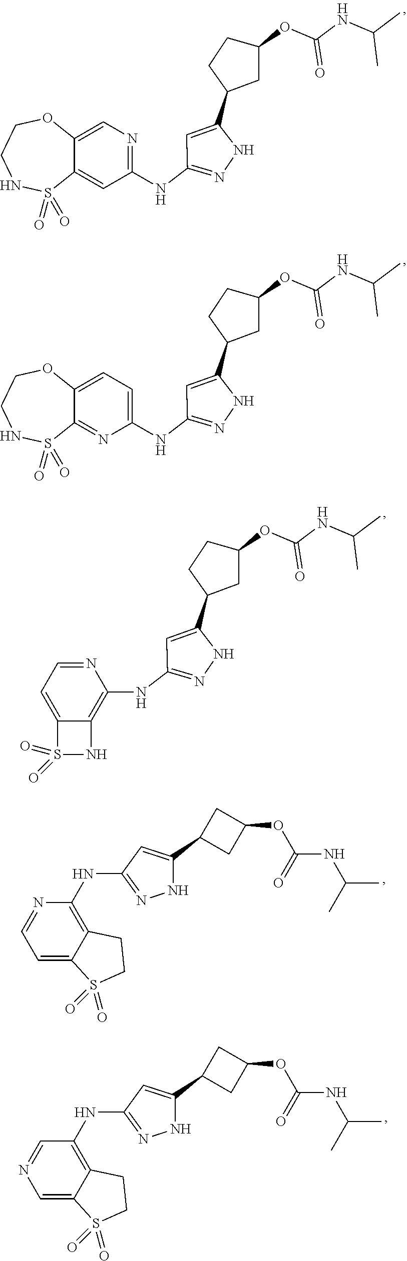 [SMILES: CC(C)NC(=O)O[C@@H]1CC[C@H](c2cc(Nc3cc4c(cn3)OCCNS4(=O)=O)n[nH]2)C1 ; CC(C)NC(=O)O[C@@H]1CC[C@H](c2cc(Nc3ccc4c(n3)S(=O)(=O)NCCO4)n[nH]2)C1 ; CC(C)NC(=O)O[C@@H]1CC[C@H](c2cc(Nc3nccc4c3NS4(=O)=O)n[nH]2)C1 ; CC(C)NC(=O)O[C@H]1C[C@@H](c2cc(Nc3nccc4c3CCS4(=O)=O)n[nH]2)C1 ; CC(C)NC(=O)O[C@H]1C[C@@H](c2cc(Nc3cncc4c3CCS4(=O)=O)n[nH]2)C1] 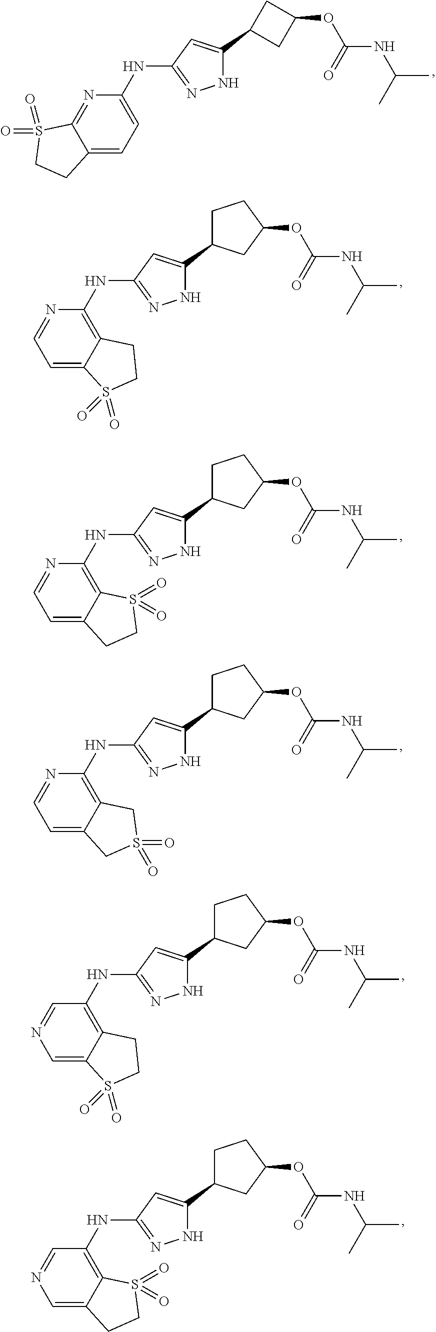 [SMILES: CC(C)NC(=O)O[C@H]1C[C@@H](c2cc(Nc3ccc4c(n3)S(=O)(=O)CC4)n[nH]2)C1 ; CC(C)NC(=O)O[C@@H]1CC[C@H](c2cc(Nc3nccc4c3CCS4(=O)=O)n[nH]2)C1 ; CC(C)NC(=O)O[C@@H]1CC[C@H](c2cc(Nc3nccc4c3S(=O)(=O)CC4)n[nH]2)C1 ; CC(C)NC(=O)O[C@@H]1CC[C@H](c2cc(Nc3nccc4c3CS(=O)(=O)C4)n[nH]2)C1 ; CC(C)NC(=O)O[C@@H]1CC[C@H](c2cc(Nc3cncc4c3CCS4(=O)=O)n[nH]2)C1 ; CC(C)NC(=O)O[C@@H]1CC[C@H](c2cc(Nc3cncc4c3S(=O)(=O)CC4)n[nH]2)C1] 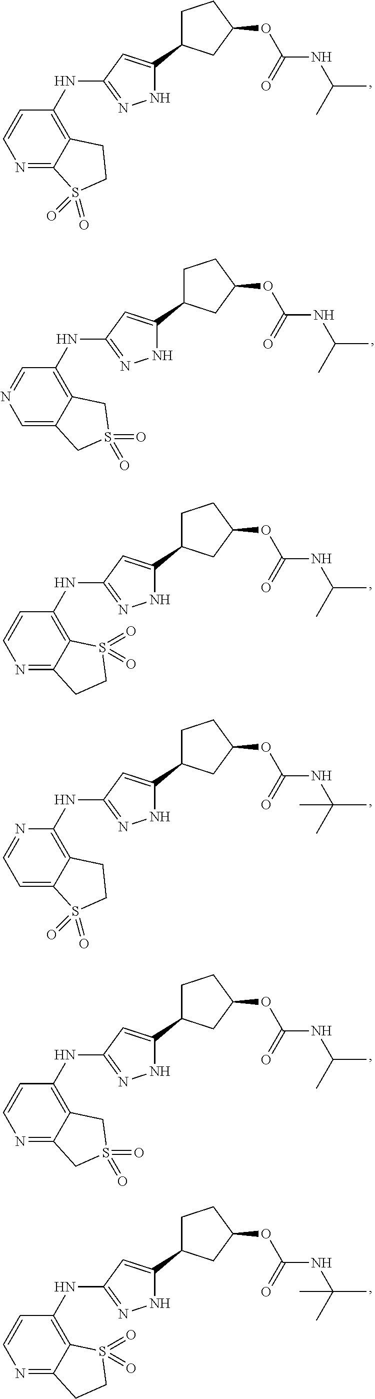 [SMILES: CC(C)NC(=O)O[C@@H]1CC[C@H](c2cc(Nc3ccnc4c3CCS4(=O)=O)n[nH]2)C1 ; CC(C)NC(=O)O[C@@H]1CC[C@H](c2cc(Nc3cncc4c3CS(=O)(=O)C4)n[nH]2)C1 ; CC(C)NC(=O)O[C@@H]1CC[C@H](c2cc(Nc3ccnc4c3S(=O)(=O)CC4)n[nH]2)C1 ; CC(C)(C)NC(=O)O[C@@H]1CC[C@H](c2cc(Nc3nccc4c3CCS4(=O)=O)n[nH]2)C1 ; CC(C)NC(=O)O[C@@H]1CC[C@H](c2cc(Nc3ccnc4c3CS(=O)(=O)C4)n[nH]2)C1 ; CC(C)(C)NC(=O)O[C@@H]1CC[C@H](c2cc(Nc3ccnc4c3S(=O)(=O)CC4)n[nH]2)C1] 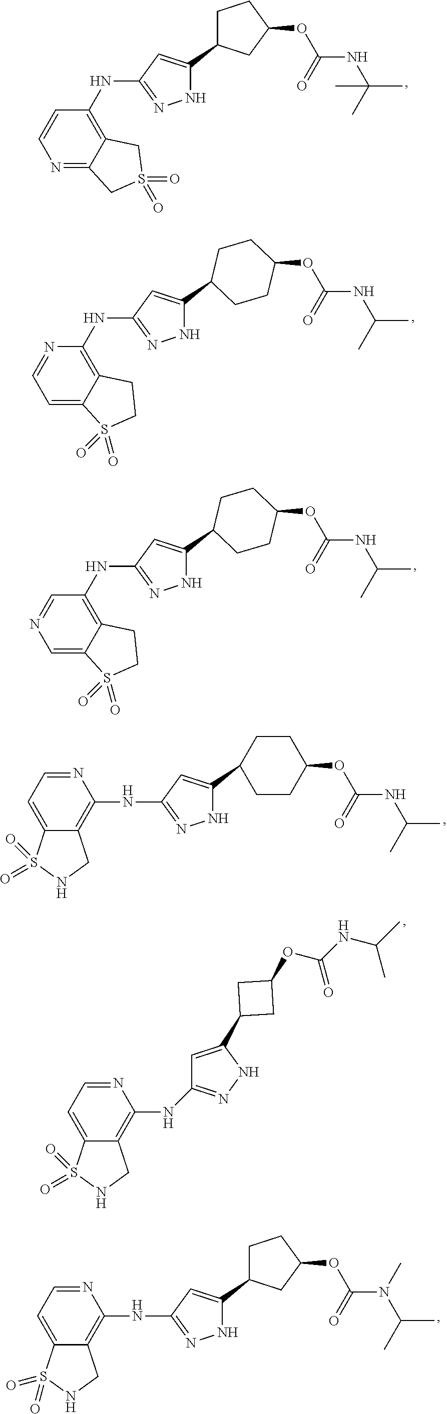 [SMILES: CC(C)(C)NC(=O)O[C@@H]1CC[C@H](c2cc(Nc3ccnc4c3CS(=O)(=O)C4)n[nH]2)C1 ; CC(C)NC(=O)O[C@H]1CC[C@@H](c2cc(Nc3nccc4c3CCS4(=O)=O)n[nH]2)CC1 ; CC(C)NC(=O)O[C@H]1CC[C@@H](c2cc(Nc3cncc4c3CCS4(=O)=O)n[nH]2)CC1 ; CC(C)NC(=O)O[C@H]1CC[C@@H](c2cc(Nc3nccc4c3CNS4(=O)=O)n[nH]2)CC1 ; CC(C)NC(=O)O[C@H]1C[C@@H](c2cc(Nc3nccc4c3CNS4(=O)=O)n[nH]2)C1 ; CC(C)N(C)C(=O)O[C@@H]1CC[C@H](c2cc(Nc3nccc4c3CNS4(=O)=O)n[nH]2)C1] 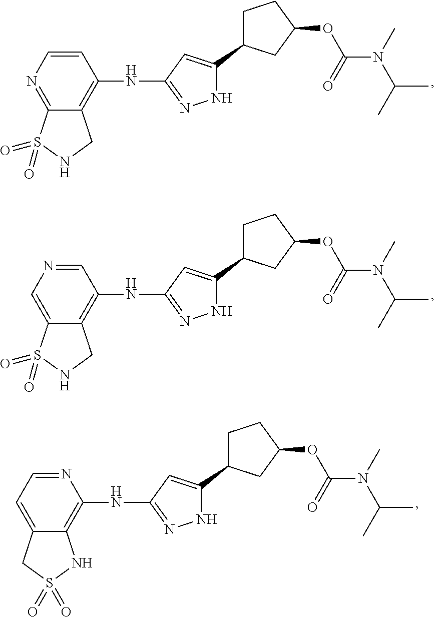 [SMILES: CC(C)N(C)C(=O)O[C@@H]1CC[C@H](c2cc(Nc3ccnc4c3CNS4(=O)=O)n[nH]2)C1 ; CC(C)N(C)C(=O)O[C@@H]1CC[C@H](c2cc(Nc3cncc4c3CNS4(=O)=O)n[nH]2)C1 ; CC(C)N(C)C(=O)O[C@@H]1CC[C@H](c2cc(Nc3nccc4c3NS(=O)(=O)C4)n[nH]2)C1] 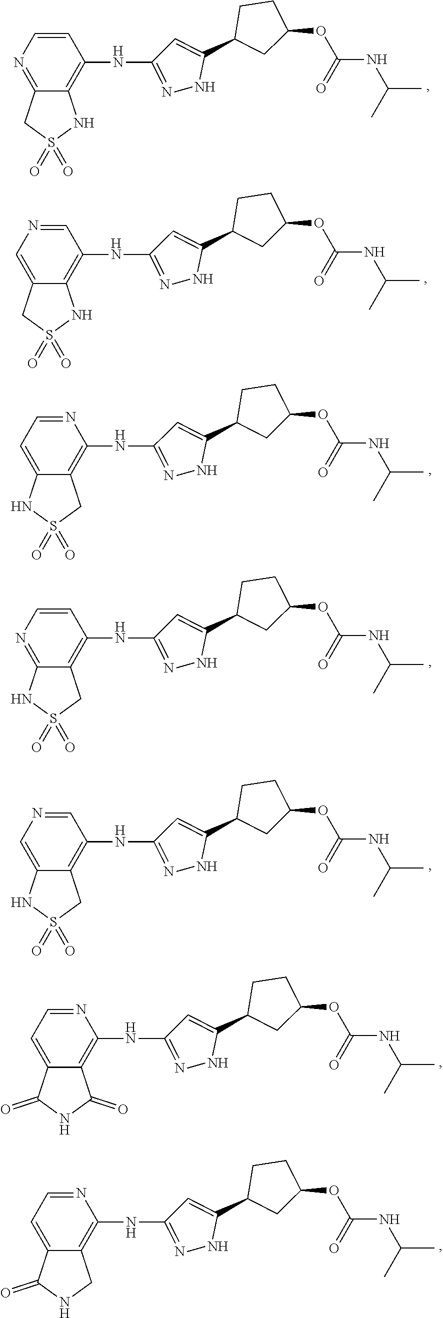 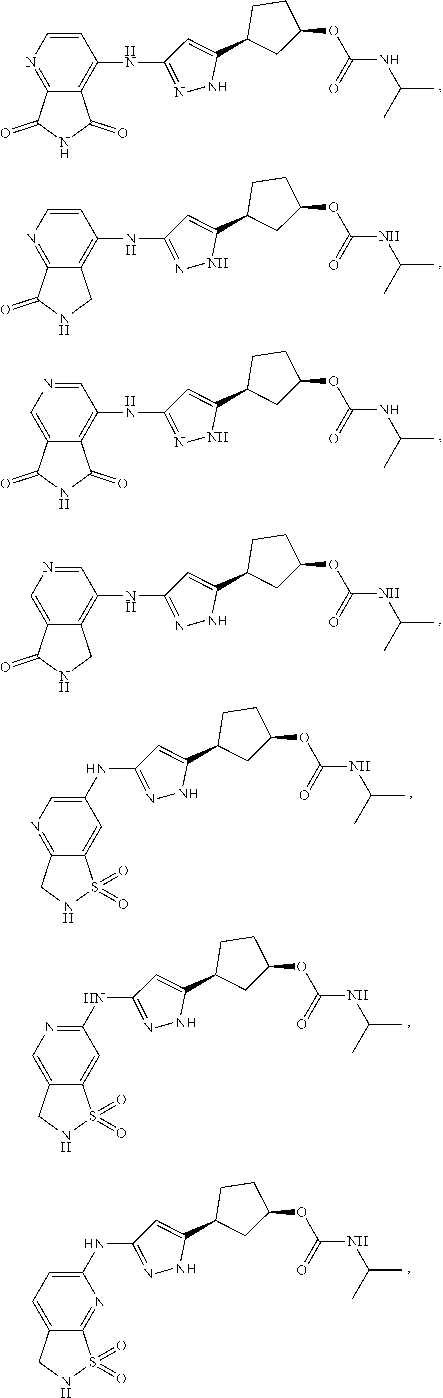 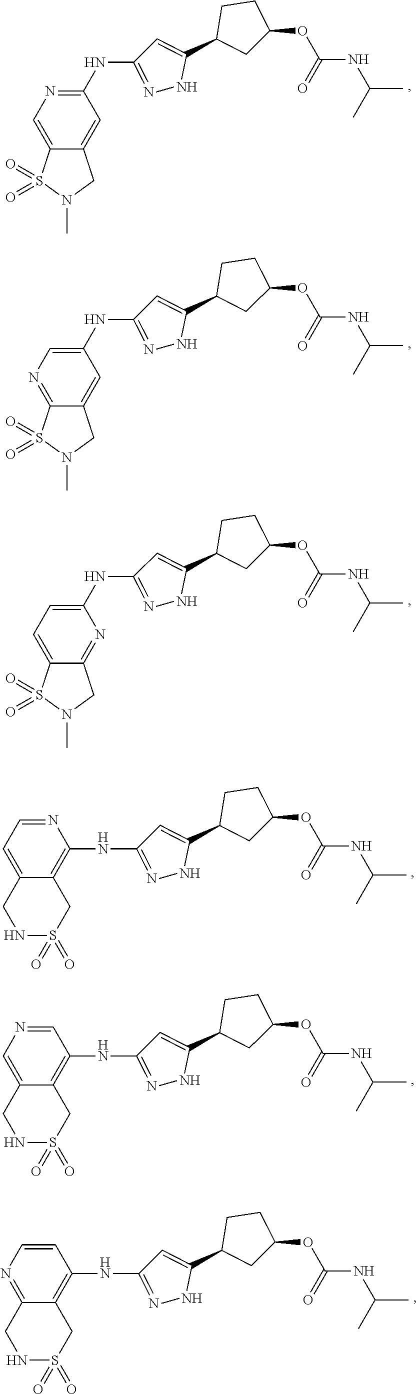 [SMILES: CC(C)NC(=O)O[C@@H]1CC[C@H](c2cc(Nc3cc4c(cn3)S(=O)(=O)N(C)C4)n[nH]2)C1 ; CC(C)NC(=O)O[C@@H]1CC[C@H](c2cc(Nc3cnc4c(c3)CN(C)S4(=O)=O)n[nH]2)C1 ; CC(C)NC(=O)O[C@@H]1CC[C@H](c2cc(Nc3ccc4c(n3)CN(C)S4(=O)=O)n[nH]2)C1 ; CC(C)NC(=O)O[C@@H]1CC[C@H](c2cc(Nc3nccc4c3CS(=O)(=O)NC4)n[nH]2)C1 ; CC(C)NC(=O)O[C@@H]1CC[C@H](c2cc(Nc3cncc4c3CS(=O)(=O)NC4)n[nH]2)C1 ; CC(C)NC(=O)O[C@@H]1CC[C@H](c2cc(Nc3ccnc4c3CS(=O)(=O)NC4)n[nH]2)C1] 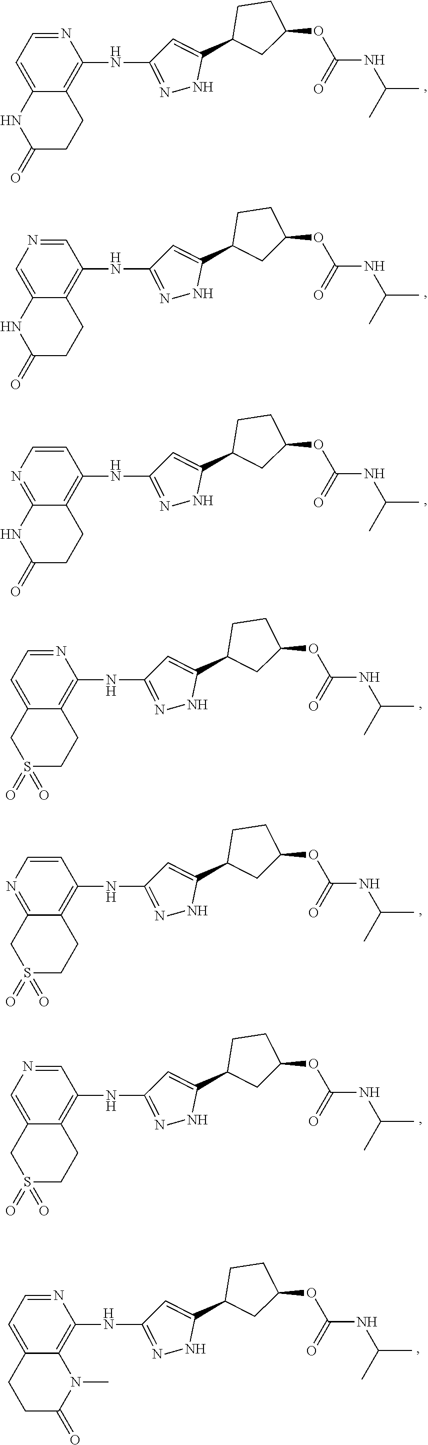 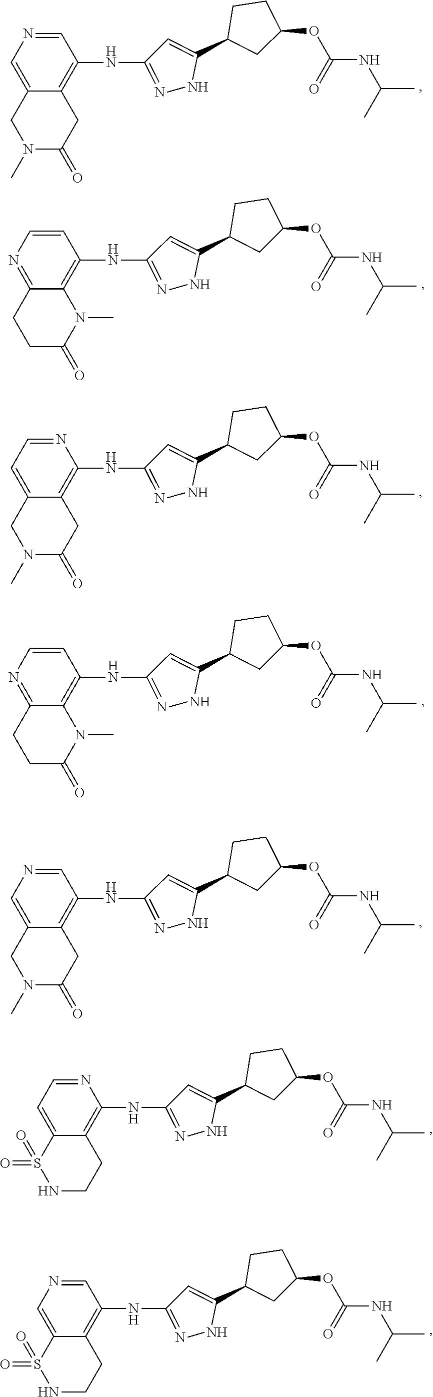 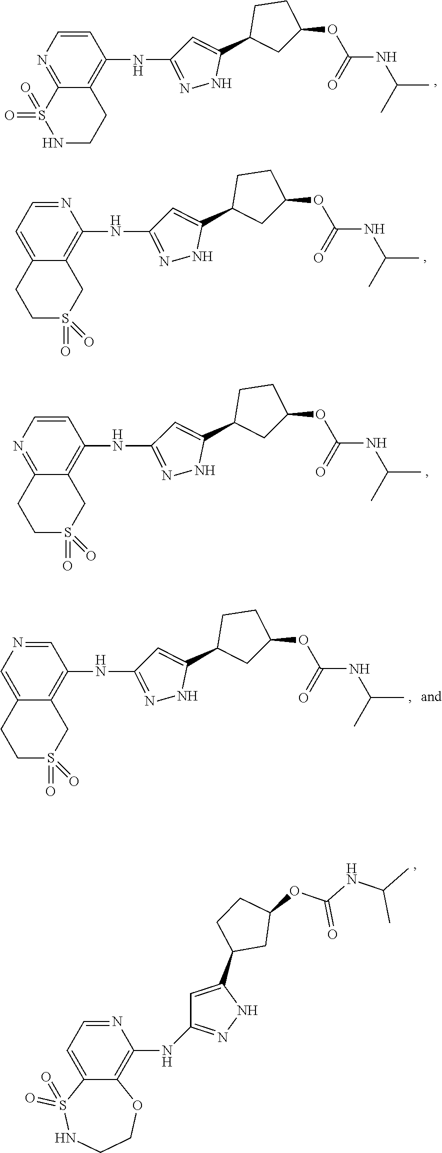 [SMILES: CC(C)NC(=O)O[C@@H]1CC[C@H](c2cc(Nc3ccnc4c3CCNS4(=O)=O)n[nH]2)C1 ; CC(C)NC(=O)O[C@@H]1CC[C@H](c2cc(Nc3nccc4c3CS(=O)(=O)CC4)n[nH]2)C1 ; CC(C)NC(=O)O[C@@H]1CC[C@H](c2cc(Nc3ccnc4c3CS(=O)(=O)CC4)n[nH]2)C1 ; CC(C)NC(=O)O[C@@H]1CC[C@H](c2cc(Nc3cncc4c3CS(=O)(=O)CC4)n[nH]2)C1 ; CC(C)NC(=O)O[C@@H]1CC[C@H](c2cc(Nc3nccc4c3OCCNS4(=O)=O)n[nH]2)C1]
or a pharmaceutically acceptable salt thereof.

105. A compound selected from 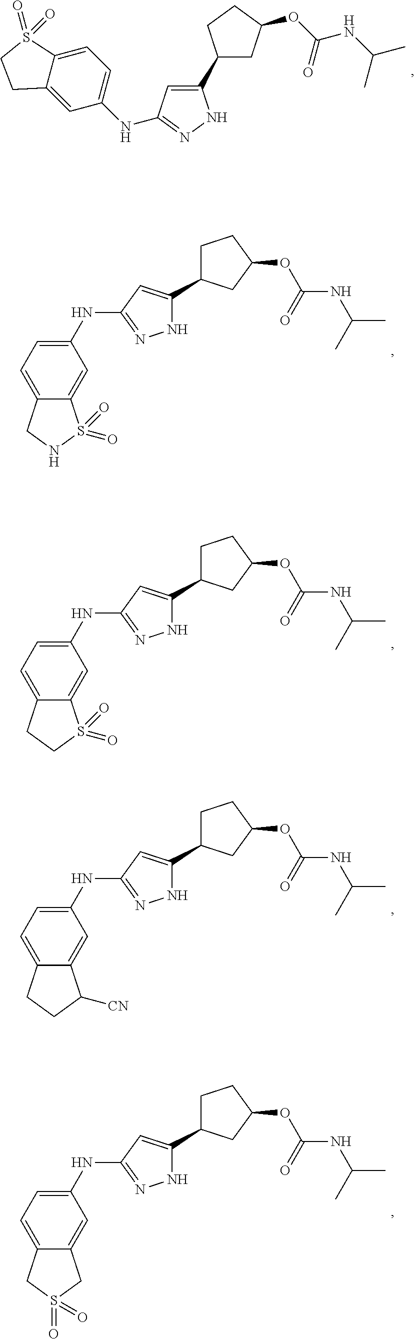 [SMILES: CC(C)NC(=O)O[C@@H]1CC[C@H](c2cc(Nc3ccc4c(c3)CCS4(=O)=O)n[nH]2)C1 ; CC(C)NC(=O)O[C@@H]1CC[C@H](c2cc(Nc3ccc4c(c3)S(=O)(=O)NC4)n[nH]2)C1 ; CC(C)NC(=O)O[C@@H]1CC[C@H](c2cc(Nc3ccc4c(c3)S(=O)(=O)CC4)n[nH]2)C1 ; CC(C)NC(=O)O[C@@H]1CC[C@H](c2cc(Nc3ccc4c(c3)C(C#N)CC4)n[nH]2)C1 ; CC(C)NC(=O)O[C@@H]1CC[C@H](c2cc(Nc3ccc4c(c3)CS(=O)(=O)C4)n[nH]2)C1] 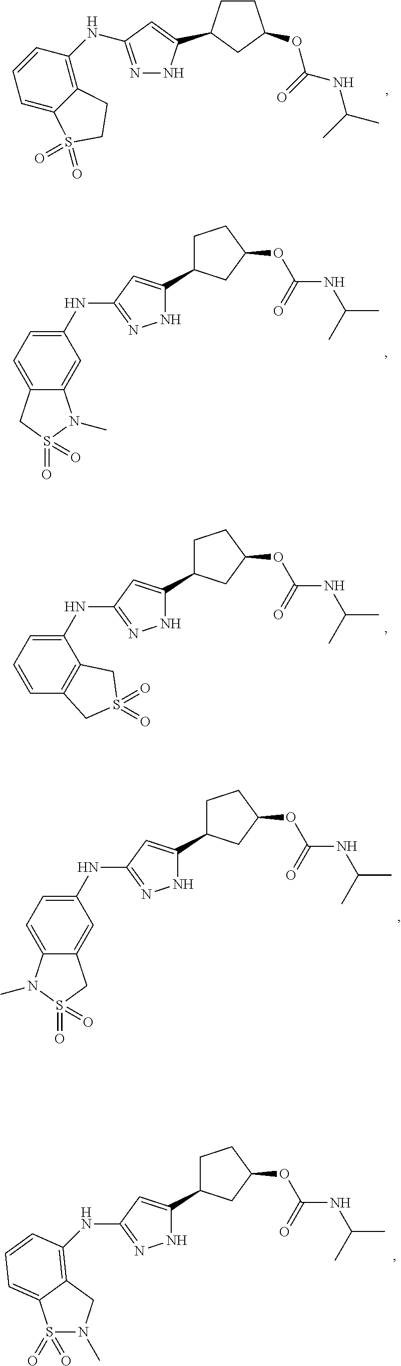 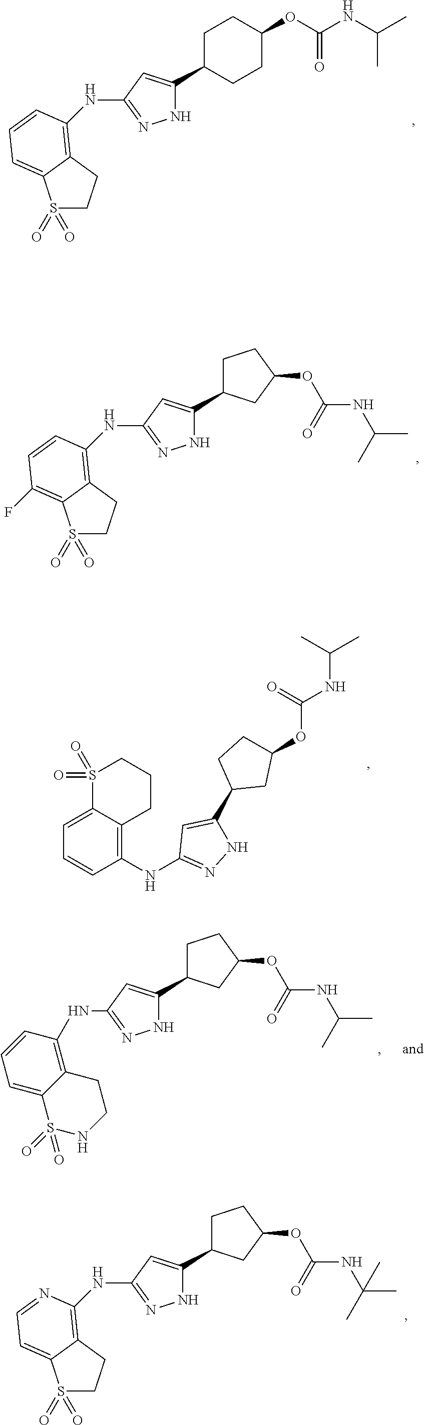 [SMILES: CC(C)NC(=O)O[C@H]1CC[C@@H](c2cc(Nc3cccc4c3CCS4(=O)=O)n[nH]2)CC1 ; CC(C)NC(=O)O[C@@H]1CC[C@H](c2cc(Nc3ccc(F)c4c3CCS4(=O)=O)n[nH]2)C1 ; CC(C)NC(=O)O[C@@H]1CC[C@H](c2cc(Nc3cccc4c3CCCS4(=O)=O)n[nH]2)C1 ; CC(C)NC(=O)O[C@@H]1CC[C@H](c2cc(Nc3cccc4c3CCNS4(=O)=O)n[nH]2)C1 ; CC(C)(C)NC(=O)O[C@@H]1CC[C@H](c2cc(Nc3nccc4c3CCS4(=O)=O)n[nH]2)C1]
or a pharmaceutically acceptable salt thereof.

106. A pharmaceutical composition comprising a compound or a pharmaceutically acceptable salt thereof of claim 90 and a pharmaceutically acceptable excipient, carrier, or diluent.

107. The pharmaceutical composition of claim 106, being suitable for oral administration.

108. A method for treating or reducing cancer, or a related disease or disorder, comprising administering to a subject in need thereof a therapeutically effective amount of a compound or a pharmaceutically acceptable salt thereof of claim 90.

109. The method of claim 108, wherein the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, gastric cancer and thyroid cancer.
